(19) United States
(12) Patent Application Publication
(10) Pub. No.: US 2004/0175722 A1
(43) Pub. Date: Sep. 9, 2004

(54) Methods and compositions for reducing screening in oligonucleotide-directed nucleic acid sequence alteration

(76) Inventors: Eric B. Kmiec, Landenberg, PA (US); Anja van Brabant, Newark, DE (US)

Correspondence Address: FISH & NEAVE, 1251 AVENUE OF THE AMERICAS, 50TH FLOOR, NEW YORK, NY, 10020-1105, US

(21) Appl. No.: 10/681,074

(22) Filed: Oct. 7, 2003

Related U.S. Application Data
(60) Provisional application No. 60/416,983, filed on Oct. 7, 2002.
(60) Provisional application No. 60/453,360, filed on Mar. 7, 2003.

(51) Int. Cl. C12Q 1/68

(52) U.S. Cl. 435/6

(57) ABSTRACT

Presented are methods, compositions and kits for reducing the number of target nucleic acid molecules required to be screened during oligonucleotide-directed nucleic acid sequence alteration.

### CROSS-REFERENCE TO RELATED APPLICATIONS

This application claims the benefit of U.S. provisional applications Nos. 60/453,360, filed Mar. 7, 2003, and 60/416,983, filed Oct. 7, 2002, the disclosures of which are incorporated herein by reference in their entireties.

### FIELD OF THE INVENTION

The technical field of the invention relates to oligonucleotide-directed alteration of nucleic acid sequence.

### BACKGROUND OF THE INVENTION

A number of methods have been developed to alter specific nucleotides within both isolated DNA molecules and DNA present within intact cells of bacteria, plants, fungi and animals, including humans.

In one approach, genomic sequences are targeted for alteration by homologous recombination using duplex fragments. The duplex fragments are large, having several hundred basepairs. See, e.g., Kunzelmann et al., Gene Ther. 3:859-867 (1996).

In another approach, oligonucleotides are used to effect targeted genetic changes.

In early experiments, oligonucleotide-directed sequence changes were typically effected in yeast, Moerschell et al., 1988, Proc. Natl. Acad. Sci. 85:524 and Yamamoto et al., Yeast 8:935 (1992), which among eukaryotes are known to have high recombinogenic activity, although one series of experiments were attempted in human cells, Campbell et al., The New Biologist 1: 223-227 (1989).

More recently, a number of different types of polynucleotides and oligonucleotides have been described that permit targeted alteration of genetic material in cells of higher eukaryotes, including (i) triplex-forming oligonucleotides; (ii) chimeric RNA-DNA oligonucleotides that are internally duplexed, notably in the region containing the nucleotide that directs the sequence alteration; and (iii) terminally modified single-stranded oligonucleotides having an internally unduplexed DNA domain and modified ends.

Sequence-altering triplexing oligonucleotides are described, for example, in U.S. Pat. Nos. 6,303,376, 5,962,426, and 5,776,744.

Triplex-forming oligonucleotides require a structural domain that binds to a DNA helical duplex through Hoogsteen interactions between the major groove of the DNA duplex and the oligonucleotide. The binding domain must typically target polypurine or polypyrimidine tracts. These sequence requirements limit the usefulness of triplex-forming oligonucleotides for targeted sequence alteration, requiring that the target sequence to be modified be situated in proximity to such polypurine or polypyrimidine tract. Triplex-forming oligonucleotides may also require an additional DNA reactive moiety, such as psoralen, to be covalently linked to the oligonucleotide, in order to stabilize the interactions between the triplex-forming domain of the oligonucleotide and the DNA double helix if the Hoogsteen interactions from the oligonucleotide/target base composition are insufficient. See, e.g., U.S. Pat. No. 5,422,251. Such DNA-reactive moieties can, however, be indiscriminately mutagenic.

In more recent work with sequence-altering triplexing oligonucleotides, the triplex-forming domain is linked or tethered to a domain that effects targeted alteration, Culver et al., Nat. Biotechnology 17: 989-93 (1999), relaxing somewhat the permissible distance between target sequence and polypurine/polypyrimidine stretch.

Internally duplexed, hairpin- and double-hairpin-containing chimeric RNA-DNA oligonucleotides are described, inter alia, in U.S. Pat. Nos. 6,573,046; 5,888,983; 5,871,984; 5,795,972; 5,780,296; 5,760,012; 5,756,325; 5,731,181, and 5,565,350. Such chimeric RNA-DNA oligonucleotides are reportedly capable of directing targeted alteration of single base pairs, as well as introducing frameshift alterations, in cells and cell-free extracts from a variety of host organisms, including bacteria, fungi, plants and animals. The oligonucleotides are reportedly able to operate on almost any target sequence.

Such chimeric molecules have significant structural requirements, however, including a requirement for both ribonucleotides and deoxyribonucleotides, and typically also a requirement that the oligonucleotide adopt a double-hairpin conformation. Even when such double hairpins are not required, however, significant structural constraints remain.

Single-stranded oligonucleotides having modified ends and an internally unduplexed DNA domain that directs sequence alteration are described in copending international patent applications published as WO 03/027265; WO 02/10364; WO 01/92512; WO 01/87914; and WO 01/73002, as well as in U.S. Pat. Nos. 6,479,292 and 6,271,360, the disclosures of which are incorporated herein by reference in their entireties.

These single-stranded oligonucleotides have fewer structural requirements than chimeric oligonucleotides and are capable of directing sequence alteration, including introduction of frameshift mutations, in cells and cell-free extracts from a variety of host organisms, including bacteria, fungi, plants and animals, in episomal and in chromosomal targets, often at alteration efficiencies that exceed those observed with hairpin-containing, internally duplexed, chimeric oligonucleotides.

The usefulness of oligonucleotide-mediated nucleic acid sequence alteration—as a means, for example, for manipulating cloned DNA, for generating agricultural products with enhanced traits, for generating cellular models for laboratory use, or for generating animal models or animals with desired traits—is affected by its frequency. The usefulness of oligonucleotide-mediated nucleic acid sequence alteration as an ex vivo or in vivo therapeutic method would also be enhanced by increasing its efficiency.

A need exists, therefore, for methods to enhance the efficiency of targeted alteration of genetic material.

### SUMMARY OF THE INVENTION

The invention provides methods, compositions and kits for enhancing oligonucleotide-directed nucleic acid sequence alteration by reducing the number of target nucleic acid molecules required to be screened during oligonucleotide-directed targeted nucleic acid sequence alteration. The methods, compositions and kits involve using at least two oligonucleotides, where at least one of the oligonucleotides directs alteration of a selectable target.

In one aspect, the invention provides methods for reducing the number of target nucleic acid molecules required to be screened during oligonucleotide-directed nucleic acid sequence alteration comprising combining a nucleic acid molecule in the presence of repair proteins with at least two oligonucleotides capable of directing alteration in at least two nucleic acid targets, where alteration by at least one oligonucleotide confers a selectable phenotype which is selected for, and selecting or screening for a nucleic acid molecule having the alteration directed by the other oligonucleotide in a composition having the selectable phenotype.

In other aspects, the invention provides compositions and kits for oligonucleotide-directed nucleic acid sequence alteration comprising at least two oligonucleotides, where at least one of the oligonucleotides directs an alteration which confers a selectable phenotype.

Other embodiments of the invention are set forth in the following numbered items:

1. A method for reducing the number of target nucleic acid molecules required to be screened during oligonucleotide-directed nucleic acid sequence alteration of a first nucleic acid target in a composition comprising said first nucleic acid target and a second nucleic acid target,

wherein alteration of said first nucleic acid target is effected by combining, in the presence of cellular repair proteins, a nucleic acid molecule comprising said first nucleic acid target with a first oligonucleotide and a second oligonucleotide,

wherein said first oligonucleotide is capable of effecting alteration of said first nucleic acid target and said second oligonucleotide is capable of effecting alteration of said second nucleic acid target, and wherein alteration of said second nucleic acid target confers a selectable phenotype,

said method comprising:

(a) selecting for alteration of said second nucleic acid target and

(b) screening for a nucleic acid molecule comprising an alteration of said first nucleic acid target.

2. A method for targeted alteration of a first nucleic acid target in a composition comprising said first nucleic acid target and a second nucleic acid target, comprising:

(a) combining, in the presence of cellular repair proteins, a nucleic acid molecule comprising said first nucleic acid target with a first oligonucleotide and a second oligonucleotide,

wherein said first oligonucleotide is capable of effecting alteration of said first nucleic acid target and said second oligonucleotide is capable of effecting alteration of said second nucleic acid target, and wherein alteration of said second nucleic acid target confers a selectable phenotype; and

(b) identifying a nucleic acid molecule comprising an alteration of said first nucleic acid target in a composition in which alteration of the second nucleic acid target has occurred.

3. The method of item 1 or 2, wherein alteration of the selectable phenotype is selected from the group consisting of: antibiotic resistance, prototrophy, expression of a fluorescent protein, presence of an epitope and resistance to an apoptotic signal.

4. The method of any one of items 1-3, wherein the alteration of the first nucleic acid target is a substitution, deletion or insertion of at least one base.

5. The method of item 4, wherein the alteration of the second nucleic acid target is a substitution, deletion or insertion of at least one base.

6. The method of any one of items 1-5, wherein the nucleic acid molecule comprising the first nucleic acid target does not comprise the second nucleic acid target.

7. The method of any one of items 1-5, wherein the nucleic acid molecule comprising the first nucleic acid target comprises the second nucleic acid target.

8. The method of any one of items 1-7, wherein the nucleic acid molecule comprising the first nucleic acid target is a DNA molecule.

9. The method of item 8, wherein the DNA molecule is selected from the group consisting of: a chromosome, a plasmid, a YAC, a BAC, a PLAC, a MAC, and a PAC.

10. The method of item 8 or 9, wherein the DNA molecule is present in a cell.

11. The method of item 10, wherein the cell is selected from the group consisting of: a prokaryotic cell, a fungal cell, a plant cell, and an animal cell.

12. The method of item 11, wherein the cell is prokaryotic cell.

13. The method of item 12, wherein the prokaryotic cell is an Escherichia Coli cell.

14. The method of item 11, wherein the cell is a fungal cell.

15. The method of item 14, wherein the fungal cell is selected from the group consisting of: a Saccharomyces cerevisiae cell, an Ustilago maydis cell, a Neurospora crassa cell and a Candida albicans cell.

16. The method of item 11, wherein the cell is a plant cell.

17. The method of item 16, wherein the plant cell is selected from the group consisting of: an angiosperm cell, a gymnosperm cell and a moss cell.

18. The method of item 16, wherein the cell is a Chlamydomonas rheinhardtii cell.

19. The method of item 17, wherein the angiosperm cell is an Arabidopsis thaliana cell.

20. The method of item 17, wherein the moss cell is a Physcomitrella patens cell.

21. The method of item 11, wherein the cell is an animal cell.

22. The method of item 21, wherein the animal cell is a mammalian cell.

23. The method of item 22, wherein the mammalian cell is selected from the group consisting of: a human cell, a rodent cell, a mouse cell, a hamster cell, a rat cell, and a monkey cell.

24. The method of item 23, wherein the human cell is selected from the group consisting of: a liver cell, a lung cell, a colon cell, a cervical cell, a kidney cell, an epithelial cell, a blood cell, a cancer cell, and a stem cell.

25. The method of any one of items 1-24, wherein the first oligonucleotide has a region that is fully complementary in sequence to the first nucleic acid target, but for one or more mismatches as between the sequences of the first oligonucleotide and its complement on the first nucleic acid target, and wherein the first oligonucleotide has at least one terminal modification.

26. The method of item 25, wherein the at least one terminal modification is selected from the group consisting of: at least one terminal locked nucleic acid (LNA), at least one terminal 2′-O-Me base analog, and at least three terminal phosphorothioate linkages.

27. The method of item 26, wherein the first oligonucleotide is a single-stranded oligonucleotide 15-121 nucleotides in length, has an internally unduplexed domain of at least 8 contiguous deoxyribonucleotides, and wherein the one or more mismatches are positioned exclusively in the oligonucleotide DNA domain and at least 7 nucleotides from said oligonucleotide's 5′ and 3′ termini.

28. The method of item 27, wherein the first oligonucleotide has at least one terminal locked nucleic acid (LNA).

29. The method of item 1 or 2, wherein the first oligonucleotide is at least 25 nucleotides in length.

30. The method of item 1 or 2, wherein the first oligonucleotide is no more than 121 nucleotides in length.

31. The method of item 30, wherein the first oligonucleotide is no more than 74 nucleotides in length.

32. A composition for targeted alteration of a first nucleic acid target, comprising:

a first oligonucleotide and a second oligonucleotide, wherein the oligonucleotides are capable, in the presence of cellular repair proteins, of effecting targeted alteration of a first nucleic target and a second nucleic acid target, respectively; and

wherein alteration of the second nucleic acid target confers a selectable phenotype.

33. The composition of item 32 further comprising cellular repair proteins.

34. The composition of item 33, wherein said cellular proteins are purified.

35. The composition of item 33, wherein said cellular proteins are present in a cell-free protein extract.

36. The composition of item 33, wherein said cellular proteins are present within an intact cell.

37. The composition of any one of items 33-36, wherein the cell is selected from the group consisting of: a prokaryotic cell, a fungal cell, a plant cell, and an animal cell.

38. The composition of any one of items 33-37, further comprising lambda beta protein.

39. The composition of any one of items 33-37, wherein the cellular proteins are derived from a cell prior-contacted with hydroxyurea.

40. The composition of any one of items 33-37, wherein the cellular proteins are derived from a cell prior-contacted with a histone deacetylase inhibitor.

41. The composition of item 37, wherein the cell is prokaryotic cell.

42. The composition of item 41, wherein the prokaryotic cell is an E. coli cell.

43. The composition of item 37, wherein the cell is a fungal cell.

44. The composition of item 43, wherein the fungal cell is selected from the group consisting of: a Saccharomyces cerevisiae cell, an Ustilago maydis cell, a Neurospora crassa cell and a Candida albicans cell.

45. The composition of item 37, wherein the cell is a plant cell.

46. The composition of item 45, wherein the plant cell is selected from the group consisting of: an angiosperm cell, a gymnosperm cell and a moss cell.

47. The composition of item 45, wherein the cell is a Chlamydomonas rheinhardtii cell.

48. The composition of item 46, wherein the angiosperm cell is an Arabidopsis thaliana cell.

49. The composition of item 46, wherein the moss cell is a Physcomitrella patens cell.

50. The composition of item 37, wherein the cell is an animal cell.

51. The composition of item 50, wherein the animal cell is a mammalian cell.

52. The composition of item 51, wherein the mammalian cell is selected from the group consisting of: a human cell, a rodent cell, a mouse cell, a hamster cell, a rat cell, and a monkey cell.

53. The composition of item 52, wherein the human cell is selected from the group consisting of: a liver cell, a lung cell, a colon cell, a cervical cell, a kidney cell, an epithelial cell, a blood cell, a cancer cell, and a stem cell.

54. The composition of any one of items 32-53, wherein the first oligonucleotide is fully complementary in sequence to the first nucleic acid target, but for one or more mismatches as between the sequences of the first oligonucleotide and its complement on the first nucleic acid target, and wherein the first oligonucleotide has at least one terminal modification.

55. The composition of item 54, wherein the at least one terminal modification is selected from the group consisting of: at least one terminal locked nucleic acid (LNA), at least one terminal 2′-O-Me base analog, and at least three terminal phosphorothioate linkages.

56. The composition of item 55, wherein the first oligonucleotide is a single-stranded oligonucleotide 15-121 nucleotides in length, has an internally unduplexed domain of at least 7 contiguous deoxyribonucleotides, and wherein the one or more mismatches are positioned exclusively in the oligonucleotide DNA domain and at least 8 nucleotides from said oligonucleotide's 5′ and 3′ termini.

57. The composition of item 55, wherein the first oligonucleotide has at least one terminal locked nucleic acid (LNA).

58. The composition of any one of items 32-57, wherein said oligonucleotide is at least 25 nucleotides in length.

59. The composition of any one of items 32-57, wherein said oligonucleotide is no more than 121 nucleotides in length.

60. The composition of item 59, wherein said oligonucleotide is no more than 74 nucleotides in length.

61. A kit for targeted alteration of nucleic acid sequence comprising:

a first oligonucleotide and a second oligonucleotide, wherein the oligonucleotides are capable, in the presence of cellular repair proteins, of effecting targeted alteration of a first nucleic target and a second nucleic acid target, respectively; and

wherein alteration of the second nucleic acid target confers a selectable phenotype.

62. The kit of item 61 further comprising a cellular repair protein.

63. The kit of item 62, wherein the cellular repair protein is from a cell precontacted with an HDAC inhibitor; hydroxyurea or lambda phage beta protein.

64. The kit of item 62, wherein the cellular repair protein is selected from the group consisting of: RAD10, RAD51, RAD52, RAD54, RAD55, MRE11, PMS1 and XRS2.

65. The kit of any one of items 61-64 further comprising an HDAC inhibitor; hydroxyurea or lambda phage beta protein.

66. The kit of item 65 further comprising a cell.

67. The kit of item 66, wherein the cell has increased levels or activity of at least one protein selected from the group consisting of: RAD10, RAD51, RAD52, RAD54, RAD55, MRE11, PMS1 and XRS2.

68. The kit of item 66 or 67, wherein the cell has decreased levels or activity of at least one protein selected from the group consisting of: RAD10, RAD51, RAD52, RAD54, RAD55, MRE11, PMS1 and XRS2.

69. The kit of any one of items 66-68, wherein the cell comprises a target nucleic acid sequence, wherein alteration of said target nucleic acid sequence by said second oligonucleotide confers a selectable phenotype.

70. The kit of any one of items 61-69, wherein the kit further comprises instructions for performing the method of item 1 or item 2.

### BRIEF DESCRIPTION OF THE DRAWINGS

FIG 1. Diagram of pAURHYG(x)eGFP target plasmids. Sequences are shown for the normal hygromycin resistance allele (SEQ ID NO: _) and the mutant alleles present in pAURHYG(rep)eGFP (SEQ ID NO:_), pAURHYG(ins)eGFP (SEQ ID NO: _) and pAURHYG(Δ)eGFP (SEQ ID NO: _).

FIG. 2. Dual targeting protocol. (A) Schematic diagram of the generalized strategy for dual targeting. (B) Sequences of the hygromycin-resistance gene and its mutation. (C) Schematic of the YAC containing the human β-globin locus and the βThal1 and βThal2 sequences that are changed by the corresponding oligonucleotides.

FIG. 3. Dual targeting results. (A) Efficiency of gene editing of hygromycin mutation using the dual targeting protocol. For these experiments, YAC-containing LSY678IntHyg(rep)βcells are grown in the presence of HU, electroporated with the selectable and nonselectable oligonucleotides, and allowed to recover in the presence of TSA. (B) Gene editing of the human β-globin gene directed by the βThal1 oligonucleotide, including the sequence of the altered segment before (SEQ ID NO: _) and after (SEQ ID NO: _) the conversion.

FIG. 4. Dual targeting and Rad51. (A) Efficiency of gene editing of hygromycin mutation using the dual targeting protocol in combination with overexpression of yeast Rad51. For these experiments, YAC-containing LSY678IntHyg(rep)β cells are grown in the presence of HU, electroporated with the selectable and nonselectable oligonucleotides, and allowed to recover in the presence of TSA. (B) Gene editing of the human β-globin gene directed by the βThal2 oligonucleotide, including the sequence of the altered segment before (SEQ ID NO: _) and after (SEQ ID NO: _) the conversion.

### DETAILED DESCRIPTION

We have discovered, surprisingly, that the frequency of oligonucleotide-directed sequence alterations at a first nucleic acid target site is higher in a population of cells that has been selected for concurrent alteration at a second nucleic acid target site, as compared to a population of cells that has not been selected for concurrent alteration at a second nucleic acid target site.

Accordingly, in a first aspect the invention provides a method for identifying cells having a desired oligonucleotide-directed sequence alteration at a first nucleic acid target site within the cell. The method comprises identifying the desired sequence alteration in cells that have been selected for the presence of a selectable phenotype conferred upon the cell by a concurrent oligonucleotide-directed sequence alteration at a second nucleic acid target site within the cell.

In a second aspect, the invention provides a method for effecting a desired sequence alteration at a first nucleic acid target site within a cell, the method comprising concurrently targeting first and second nucleic acid sites within the cell for sequence alteration with respective first and second sequence-altering oligonucleotides, the second alteration conferring a selectable phenotype upon the cell; selecting cells having the selectable phenotype; and then identifying among the selected cells those having the desired sequence alteration at the first nucleic acid target site.

The methods of the present invention increase the efficiency with which bacteria, plant, fungi and animal cells having a desired genotypic change at the first target site may be identified. In related aspects, the invention provides compositions and kits for effecting or facilitating practice of the methods of the present invention.

Either or both of the first and second nucleic acid target sites within the cell may be in genomic double-stranded DNA.

The targeted genomic DNA can be normal, cellular chromosomal DNA; organellar DNA, such as mitochondrial or plastid DNA; or extrachromosomal DNA present in cells in different forms including, e.g., mammalian artificial chromosomes (MACs), PACs from P-1 vectors, yeast artificial chromosomes (YACs), bacterial artificial chromosomes (BACs), plant artificial chromosomes (PLACs), BiBACS, as well as episomal DNA, including episomal DNA from an exogenous source such as a plasmid or recombinant vector. Many of these artificial chromosome constructs containing human DNA can be obtained from a variety of sources, including, e.g., the Whitehead Institute, and are described, e.g., in Cohen et al., Nature 336: 698-701 (1993) and Chumakov, et al., Nature 377: 175-297 (1995).

The first targeted nucleic acid site may be in a part of the DNA that is transcriptionally silent or transcriptionally active; typically, the second targeted nucleic acid site will be in a part of the DNA that is transcriptionally active so as to confer a selectable phenotype upon the cell. The first and second targeted sites may be in any part of a gene including, for example, an exon, an intron, a promoter, an enhancer or a 3′- or 5′-untranslated region, and may be in intergenic regions, with the second targeted site typically being in an exon so as to confer a selectable phenotype upon the cell.

In some embodiments, the first and/or second sequence-altering oligonucleotide is designed to direct alteration of the transcribed strand of the target sequence; in other embodiments, the first and/or second oligonucleotide is designed to direct alteration of nucleic acid sequence targeting the non-transcribed strand of the target sequence. The targeted strand may differ as between first and second target sites.

The first and second oligonucleotides may independently be selected from any type of sequence-altering oligonucleotide known in the art, including (i) triplex-forming oligonucleotides; (ii) chimeric RNA-DNA oligonucleotides that are internally duplexed, notably in the region containing the nucleotide that directs the sequence alteration; and (iii) terminally modified single-stranded oligonucleotides having an internally unduplexed DNA domain and modified ends.

Sequence-altering triplexing oligonucleotides useful in the methods, compositions, and kits of the present invention are described, for example, in U.S. Pat. Nos. 6,303,376, 5,962,426, and 5,776,744, the disclosures of which are incorporated herein by reference in their entireties. Bifunctional oligonucleotides having a triplex-forming domain linked or tethered to a domain that effects targeted alteration, useful in the methods, compositions, and kits of the present invention, are described in Culver et al., Nat. Biotechnology 17: 989-93 (1999), the disclosure of which is incorporated herein by reference in its entirety.

Internally duplexed, hairpin- and double-hairpin-containing chimeric RNA-DNA oligonucleotides useful in the methods, compositions, and kits of the present invention are described, inter alia, in U.S. Pat. Nos. 6,573,046; 5,888,983; 5,871,984; 5,795,972; 5,780,296; 5,760,012; 5,756,325; 5,731,181, and 5,565,350, the disclosures of which are incorporated herein by reference in their entireties.

In preferred embodiments, at least one of the first and second oligonucleotides is a single-stranded oligonucleotide having modified ends and an internally unduplexed DNA domain that directs sequence alteration.

Such oligonucleotides are further described in copending international patent applications published as WO 03/027265; WO 02/10364; WO 01/92512; WO 01/87914; and WO 01/73002, as well as in U.S. Pat. Nos. 6,479,292 and 6,271,360, the disclosures of which are incorporated herein by reference in their entireties.

In typical embodiments, the oligonucleotide is 17-121 nucleotides in length and has an internally unduplexed domain (that is, a nonhairpin domain) of at least 8 contiguous deoxyribonucleotides. The oligonucleotide is fully complementary in sequence to the sequence of a first strand of the respective nucleic acid target, but for one or more mismatches as between the sequences of the oligonucleotide internally unduplexed deoxyribonucleotide domain and its complement on the target nucleic acid first strand. Each of the mismatches is positioned at least 8 nucleotides from each of the oligonucleotide's 5′ and 3′ termini. The oligonucleotide has at least one terminal modification.

In some embodiments, the at least one terminal modification may be selected from the group consisting of 2′-O-alkyl, such as 2′-O-methyl, residue; phosphorothioate internucleoside linkage; and locked nucleic acid (LNA) residue. The basic structural and functional characteristics of LNAs and related analogues are disclosed in various publications and patents, including WO 99/14226, WO 00/56748, WO 00/66604, WO 98/39352, U.S. Pat. No. 6,043,060, and U.S. Pat. No. 6,268,490, the disclosures of which are incorporated herein by reference in their entireties. In some embodiments, the terminal modification comprises a plurality of adjacent phosphorothioate internucleoside linkages, such as three phosphorothioate linkages at the 3′ terminus of the oligonucleotide.

In certain preferred embodiments, both of the first and second sequence-altering oligonucleotides are single-stranded oligonucleotides having modified ends and an internally unduplexed DNA domain that directs sequence alteration.

In some embodiments, a plurality of single-stranded oligonucleotides having modified ends and an internally unduplexed DNA domain that directs sequence alteration can be used to effect either or both of the first and second sequence alterations. Use of such plural oligonucleotides is described in copending U.S. patent application No. 10/623,107, filed Jul. 18, 2003 (“Targeted Nucleic Acid Sequence Alteration Using Plural Oligonucleotides”), the disclosure of which is incorporated herein by reference in its entirety.

In the methods, compositions, and kits of the present invention, at least the second oligonucleotide directs a sequence alteration that produces a selectable phenotype. Although the first oligonucleotide may also direct an alteration that produces a selectable phenotype, generally the first oligonucleotide directs an alteration that must be identified by screening, e.g., by determining the corresponding nucleic acid sequence or by assaying a non-selectable phenotype that is generated by the alteration event.

The selectable phenotype chosen will depend on the host cell chosen and whether the selection is effected in vitro or in vivo. As is well known in the art, exemplary selectable phenotypes include, e.g., antibiotic or other chemical resistance, ability to use a nutrient source, expression of a fluorescent protein, presence of an epitope or resistance to an apoptotic signal. The selectable phenotype chosen may be selectable based on preferential growth of a cell with the desired sequence alteration. Examples of such selectable phenotypes include, e.g., the ability to grow in the presence of a compound that either kills or prevents the growth of the cell such as an apoptotic signal or an antibiotic, the ability to grow in the absence of a nutrient that is required prior to the sequence alteration, or the ability to utilize a particular resource that is not usable prior to the sequence alteration. The selectable phenotype may also be selected mechanically. Examples of phenotypes that may be selected mechanically include, e.g., expression of a fluorescent protein or a particular epitope. Mechanical selection may be by any means known to one of skill in the art including, e.g., FACS (directly in the case of a fluorescent protein or using a labeled antibody for an epitope), column chromatography, or using paramagnetic beads produced by, e.g., Miltenyi Biotec. Selection also does not require intact cells. For example, a single nucleotide change (SNP) in a nucleic acid molecule may be detected and isolated in vitro using methods such as are described in WO 03/027640. In such cases, the first oligonucleotide effects a change in the selected molecule.

The methods, compositions and kits of the invention typically reduce the number of cells required to be screened by at least about two-fold relative to the number that must be screened in a population of targeted cells that has not previously been selected for an oligonucleotide-directed nucleic acid sequence alteration that confers a selectable phenotype. The reduction can be by at least about two, three, four, five, six, seven, eight, nine, ten, twelve, fifteen, twenty, thirty, and fifty or more fold.

The methods, compositions and kits of the invention may be used with any oligonucleotide that directs targeted alteration of nucleic acid sequence. In the examples and in Tables 5 to 42 herein, correcting oligonucleotides of defined sequence are provided for alteration of human genes, including correction of genes mutated in human diseases, and for directing specific alterations in plant genes. Each of these oligonucleotides may be a first sequence-altering oligonucleotide as defined herein. Certain of these oligonucleotides may also be a second sequence-altering oligonucleotide as defined herein, e.g., where the oligonucleotide effects a nucleic acid sequence alteration that confers a selectable phenotype such as herbicide resistance. In the tables of these examples, the oligonucleotides are not limited to the particular sequences disclosed. The oligonucleotides include extensions of the appropriate sequence of the longer 121 base oligonucleotides which can be added base by base to the smallest disclosed oligonucleotides of 17 bases. Some oligonucleotides may be 15 or 16 bases which can be obtained by subtraction or one or two bases from the smallest disclosed oligonucleotides of 17 bases. Thus the representative oligonucleotides include for each correcting change, oligonucleotides of length 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 with further single-nucleotide additions up to the longest sequence disclosed. These oligonucleotide sequences can be used to design first oligonucleotides, or, where the oligonucleotide directs an alteration that confers a selectable phenotype, first and/or second oligonucleotides. Moreover, the oligonucleotides of the invention do not require a symmetrical extension on either side of the central DNA domain. Similarly, the oligonucleotides designed using the sequences of oligonucleotides disclosed in the various tables for correction of human diseases or for directing specific alterations in plant genes comprise structures or modifications that enable them to effect oligonucleotide-directed nucleic acid sequence alteration, such as, e.g., phosphorothioate linkages, LNA residues or chimeric RNA-DNA internally duplexed structure.

Efficiency of conversion is defined herein as the percentage of recovered substrate target molecules that have undergone a conversion event. Depending on the nature of the target genetic material, e.g. an extrachromosomal element in a cell, efficiency could be represented as the proportion of cells or clones containing an extrachromosomal element that exhibit a particular phenotype. Alternatively, representative samples of the target genetic material can be analyzed, e.g. by sequencing, allele-specific PCR or comparable techniques, to determine the percentage that have acquired the desired change. This latter method of determining efficiency is most frequently applied where the phenotype conferred by the alteration is a non-selectable phenotype.

Each of the first and second oligonucleotides can direct any kind of alteration, including, for example, deletion, insertion or replacement of 1, 2 or 3 nucleotides in the target sequence. These altered nucleotides may be contiguous or non-contiguous to each other. Multiple alterations can be directed to each of the first and second target sites by a single oligonucleotide or by 1, 2 or 3 separate oligonucleotides. In some embodiments, the multiple alterations are directed by a single oligonucleotide. In some embodiments, the multiple alterations are within 1 to 10 nucleotides of each other.

The methods, compositions and kits of the invention can be combined with one or more other methods of enhancing the efficiency of oligonucleotide-directed alteration of nucleic acid sequence known in the art.

Such methods are described, e.g., in copending International patent applications published as WO 02/10364 (“Methods for Enhancing Targeted Gene Alteration Using Oligonucleotides,”); WO 03/027265 (“Composition and Methods for Enhancing Oligonucleotide-Directed Sequence Alteration”); and WO 03/075856 (“Methods, Compositions, and Kits for Enhancing Oligonucleotide-Mediated Nucleic Acid Sequence Alteration Using Compositions Comprising a Histone Deacetylase Inhibitor, Lambda Phage Beta Protein, or Hydroxyurea”), the disclosures of which are incorporated herein by reference in their entireties.

For example, in typical embodiments of this aspect of the invention, the methods comprise treating a cell or tissue from a bacterium, a fungus, a plant, or an animal with a histone deacetylase (HDAC) inhibitor or hydroxyurea (HU), and then administering to the treated cell or tissue at least first and second oligonucleotides having nucleic acid sequence alteration activity. In other embodiments, the HDAC inhibitor or hydroxyurea, respectively, may be added contemporaneously with oligonucleotide addition or even following oligonucleotide addition.

The HDAC inhibitor can be trichostatin A.

One of skill in the art will appreciate, however, that other HDAC inhibitors may be suitable for these purposes. For example, U.S. patent application Publication No. 2002/0143052, which is hereby incorporated by reference in its entirety, discloses compounds having HDAC inhibitor activity due to the presence of a zinc-binding moiety. Other examples of HDAC inhibitors suitable for purposes of the invention include butyric acid, MS-27-275, suberoylanilide hydroxamic acid (SAHA), oxamflatin, trapoxin A, depudecin, FR901228 (also known as depsipeptide), apicidin, m-carboxy-cinnamic acid bishydroxamic acid (CBHA), suberic bishydroxamic acid (SBHA), and pyroxamide. See Marks et al., J. Natl. Canc. Inst. 92(15):1210-1216 (2000), which is hereby incorporated by reference in its entirety. Yet other examples of suitable HDAC inhibitors are chlamydocin, HC-toxin, Cyl-2, WF-3161, and radicicol, as disclosed in WO 00/23567, which is hereby incorporated by reference in its entirety.

When administering an HDAC inhibitor or HU to cells or cell extracts, the dosage to be administered and the timing of administration will depend on various factors, including cell type.

In the case of TSA, the dosage may be 10 nM, 100 nM, 1 μM, 10 μM, 100 μM, 1 mM, 10 mM, or even higher, or as little as 1 mM, 100 μM, 10 μM, 1 μM, 100 nM, 10 nM, 1 nM, or even lower. In the case of HU, the dosage may be 100 nM, 1 μM, 10 μM, 100 μM, 1 mM, 10 mM, 100 mM, 1 or even higher, or as little as 100 mM, 10 mM, 1 mM, 100 μM, 10 μM, 1 μM, 100 nM, 10 nM, or even lower.

In the case of HU, treatment may be with 100 mM, 75 mM, 50 mM, 40 mM, 20 mM, 10 mM, 2 mM, 1 mM, 100 microM, 10 microM, 1 microM, 100 nM, 10 nM or lower. The dosage is preferably from about 4 to 100 mM for yeast cells and from about 0.05 mM to 3 mM for mammalian cells. The dosage may be at least 0.05 mM, 0.10 mM, 0.15 mM, 0.20 mM, 0.25 mM, 0.30 mM, 0.35 mM, 0.40 mM, 0.50 mM or more, including at least 0.55 mM, 0.60 mM, 0.65 mM, 0.70 mM, 0.75 mM, 0.80 mM, mM, 0.90 mM, 0.95 mM or even 1 mM, 1.1 mM, 1.2 mM, 1.3 mM, 1.4 mM, 1.4 mM, 1.5 mM, 1.6 mM, 1.7 mM, 1.8 mM, 1.9 mM, 2.0 mM, 2.5 mM, 3 mM, or more. Typically, the dosage for mammalian cells is less than about 3.0 mM, and can be less than 2.5 mM, 2.0 mM, 1.5 mM, 1.0 mM, even less than 0.90, 0.85, 0.80, 0.75, 0.70, 0.65, 0.60, 0.55, 0.50, 0.45, 0.40, and even less than about 0.35 or 0.30 mM.

Cells may be grown in the presence of an HDAC inhibitor or HU, and cell extracts may be treated with the HDAC inhibitor or HU, for various times prior to combination with a sequence-altering oligonucleotide. Growth or treatment may be as long as 1 h, 2 h, 3 h, 4 h, 6 h, 8 h, 12 h, 20 h, or even longer, including up to 28 days, 14 days, 7 days, or shorter, or as short as 12 h, 8 h, 6 h, 4 h, 3 h, 2 h, 1 h, or even shorter. Alternatively, treatment of cells or cell extracts with HDAC inhibitor or HU and the sequence-altering oligonucleotide may occur simultaneously, or the HDAC inhibitor or HU, respectively, may be added after oligonucleotide addition.

Cells may further be allowed to recover from treatment with an HDAC inhibitor or HU by growth in the absence of the HDAC inhibitor or HU for various times prior to treatment with a sequence-altering oligonucleotide. Recovery may be as long as 10 min, 20 min, 40 min, 60 min, 90 min, 2 h, 4 h, or even longer, or as short as 90 min, 60 min, 40 min, 20 min, 10 min, or even shorter. Cells may also be allowed to recover following their treatment with a sequence-altering oligonucleotide. This recovery period may be as long as 1 h, 2 h, 4 h, 6 h, 8 h, 12 h, or even longer, or as short as 8 h, 6 h, 4 h, 2 h, 1 h, or even shorter. The HDAC inhibitor or HU may either be present in or absent from the cell medium during the recovery period.

Optimum dosages and the timing and duration of administration of HDAC inhibitors and HU to cells or cell extracts can be determined by routine experimentation. For example, optimized dosage and timing of treatment with an HDAC inhibitor, such as TSA, can be determined using the assay system described in WO 03/075856.

Cultured cells (such as yeast cells) are treated with varying concentrations of HDAC inhibitor for a varying number of hours prior to electroporation with the sequence altering oligonucleotide. After recovery for varying periods, the cells are plated and tested for efficiency of sequence alteration. Parameters are then selected that provide the highest efficiency of correction. The method may then be repeated, as necessary, further to optimize dosage, duration of pretreatment, duration of recovery period, if any, and the like.

A similar approach for HU can be determined using the assay system set forth in WO 03/075856.

The methods, compositions, and kits of the instant invention comprising either an HDAC inhibitor, such as trichostatin A, or HU typically increase nucleic acid sequence alteration efficiency by at least two fold relative to the same method respectively lacking the HDAC inhibitor or HU. The increase in nucleic acid sequence alteration efficiency can also be about three, four, five, six, seven, eight, nine, ten, twelve, fifteen, twenty, thirty, and fifty or more fold. The methods, compositions, and kits of the instant invention comprising beta protein increase the efficiency of altering a DNA sequence, as compared to the same method lacking beta protein, typically at least 2 fold, and can increase the efficiency 5 fold, 10 fold, 15 fold, 20 fold, 25 fold, 30 fold, 40 fold, 50 fold, 60 fold, 70 fold, 80 fold, 90 fold, 100 fold, 250 fold, 500 fold, 1000 fold, or more; in certain embodiments, the methods, compositions, and kits of the instant invention that comprise beta protein increase efficiency less than two-fold as compared to comparable methods lacking beta protein, such as 1.9 fold, 1.5 fold, or even by 10%, 20%, 30%, 40%.

The cells in which targeted nucleic acid sequence alterations may usefully be made according to the methods of the present invention include mammalian cells, including human cells, such as liver, lung, colon, cervix, kidney, and epithelium cells.

Cultured mammalian cells that usefully may be targeted for desired sequence alteration according to the methods of the present invention include HT1080 cells (human epithelial fibrosarcoma), COS-1 and COS-7 cells (African green monkey), CHO-K1 cells (Chinese hamster ovary), H1299 cells (human epithelial carcinoma, non-small cell lung cancer), C127I (immortal murine mammary epithelial cells), MEF (mouse embryonic fibroblasts), HEC-1-A (human uterine carcinoma), HCT15 (human colon cancer), HCT116 (human colon carcinoma), LoVo (human colon adenocarcinoma), and HeLa (human cervical carcinoma) cancer cells as well as PC12 cells (rat pheochromocytoma).

Alterations in cultured mammalian cells may usefully be made to create coisogenic cell collections, as described in copending international patent application published as WO 03/027264 and U.S. patent application Ser. No. 10/260,638, the disclosures of which are incorporated herein by reference in their entireties. Genes usefully targeted in such coisogenic collections include loci affecting drug resistance (equivalently, drug sensitivity) or drug metabolism, including: CYP1A2, CYP2C17, CYP2D6, CYP2E, CYP3A4, CYP4A11, CYP1B1, CYP1A1, CYP 2A6, CYP2A13, CYP2B6, CYP2C8, CYP2C9, CYP11A, CYP2C19, CYP2F1, CYP2J2, CYP3A5, CYP3A7, CYP4B1, CYP4F2, CYP4F3, CYP6D1, CYP6F1, CYP7A1, CYP8, CYP11A, CYP11B1, CYP11B 2, CYP17, CYP19, CYP21A2, CYP24, CYP27A1, CYP51, ABCB1, ABCB4, ABCC1, ABCC2, ABCC3, ABCC4, ABCC5, ABCC6, MRP7, ABCC8, ABCC9, ABCC10, ABCC11, ABCC12, EPHX1, EPHX2, LTA4H, TRAG3, GUSB, TMPT, BCRP, HERG, hKCNE2, UDP glucuronosyl transferase (UGT), sulfotransferase, sulfatase, glutathione S-transferase (GST)—alpha, glutathione S-transferase—mu, glutathione S-transferase—pi, ACE, and KCHN2.

In other embodiments, cells within which targeted alterations may usefully be effected according to the methods of the present invention include progenitor and stem cells—both embryonic (ES) stem cells and non-ES cells such as hematopoietic progenitor or stem cells, including CD34⁺CD38⁻ hematopoietic progenitor and stem cells and muscle-derived stem cells.

ES cells can be mammalian ES cells, either non-human mammalian ES cells or human ES cells; human ES cells may, e.g., be from a cell line approved for use in the jurisdiction in which the methods, compositions and kits of the present invention are to be used. For example, for use in the United States, any human stem cell line that does not violate state or federal law may be used, such as those cell lines that meet United States federal funding criteria; the National Institutes of Health is currently compiling a list of these existing stem cell lines (http://escr.nih.gov) which includes those held by the following: BresaGen, Inc., Athens, Ga. (4 lines); CyThera, Inc., San Diego, Calif. (9 lines); Karolinska Institute, Stockholm, Sweden (5 lines); Monash University, Melbourne, Australia (6 lines); National Center for Biological Sciences, Bangalore, India (3 lines); Reliance Life Sciences, Mumbai, India (7 lines); Technion-Israel Institute of Technology, Haifa, Israel (4 lines); University of California, San Francisco, Calif. (2 lines); Göteborg University, Goteborg, Sweden (19 lines); Wisconsin Alumni Research Foundation, Madison, Wis. (5 lines).

In yet other embodiments, the cells within which targeted alterations are made are plant cells.

Particularly useful plants from which the cells to be used may be drawn include, for example, experimental model plants such as Chlamydomonas reinhardtii, Physcomitrella patens, and Arabidopsis thaliana in addition to crop plants such as cauliflower (Brassica oleracea), artichoke (Cynara scolymus), fruits such as apples (Malus, e.g. domesticus), mangoes (Mangifera, e.g. indica), banana (Musa, e.g. acuminata), berries (such as currant, Ribes, e.g. rubrum), kiwifruit (Actinidia, e.g. chinensis), grapes (Vitis, e.g. vinifera), bell peppers (Capsicum, e.g. annuum), cherries (such as the sweet cherry, Prunus, e.g. avium), cucumber (Cucumis, e.g. sativus), melons (Cucumis, e.g. melo), nuts (such as walnut, Juglans, e.g. regia; peanut, Arachis hypogeae), orange (Citrus, e.g. maxima), peach (Prunus, e.g. persica), pear (Pyra, e.g. communis), plum (Prunus, e.g. domestica), strawberry (Fragaria, e.g. moschata or vesca), tomato (Lycopersicon, e.g. esculentum); leaves and forage, such as alfalfa (Medicago, e.g. sativa or truncatula), cabbage (e.g. Brassica oleracea), endive (Cichoreum, e.g. endivia), leek (Allium, e.g. porrum), lettuce (Lactuca, e.g. sativa), spinach (Spinacia, e.g. oleraceae), tobacco (Nicotiana, e.g. tabacum); roots, such as arrowroot (Maranta, e.g. arundinacea), beet (Beta, e.g. vulgaris), carrot (Daucus, e.g. carota), cassava (Manihot, e.g. esculenta), turnip (Brassica, e.g. rapa), radish (Raphanus, e.g. sativus), yam (Dioscorea, e.g. esculenta), sweet potato (Ipomoea batatas); seeds, including oilseeds, such as beans (Phaseolus, e.g. vulgaris), pea (Pisum, e.g. sativum), soybean (Glycine, e.g. max), cowpea (Vigna unguiculata), mothbean (Vigna aconitifolia), wheat (Triticum, e.g. aestivum), sorghum (Sorghum e.g. bicolor), barley (Hordeum, e.g. vulgare), corn (Zea, e.g. mays), rice (Oryza, e.g. sativa), rapeseed (Brassica napus), millet (Panicum sp.), sunflower (Helianthus annuus), oats (Avena sativa), chickpea (Cicer, e.g. arietinum); tubers, such as kohlrabi (Brassica, e.g. oleraceae), potato (Solanum, e.g. tuberosum) and the like; fiber and wood plants, such as flax (Linum e.g. usitatissimum), cotton (Gossypium e.g. hirsutum), pine (Pinus sp.), oak (Quercus sp.), eucalyptus (Eucalyptus sp.), and the like and ornamental plants such as turfgrass (Lolium, e.g. rigidum), petunia (Petunia, e.g. x hybrida), hyacinth (Hyacinthus orientalis), carnation (Dianthus e.g. caryophyllus), delphinium (Delphinium, e.g. ajacis), Job's tears (Coix lacryma-jobi), snapdragon (Antirrhinum majus), poppy (Papaver, e.g. nudicaule), lilac (Syringa, e.g. vulgaris), hydrangea (Hydrangea e.g. macrophylla), roses (including Gallicas, Albas, Damasks, Damask Perpetuals, Centifolias, Chinas, Teas and Hybrid Teas) and ornamental goldenrods (e.g. Solidago spp.).

Generally, the oligonucleotides are administered to isolated plant cells or protoplasts according to a method of the present invention and the resulting cells are used to regenerate whole plants according to any method known in the art.

Relatively few specific plant mutations that produce desirable phenotypes have been described for plant species or cultivars.

However, the methods, compositions and kits of the instant invention may be used to identify a desirable mutation in one species, for example an experimental model plant, and the desirable mutation can then be introduced in the homologous genes of other species using the kits, compositions and methods of the invention. Further, the methods, compositions and kits of the invention can be used to produce “knock out” mutations by modification of specific amino acid codons to produce stop codons (e.g., a CAA codon specifying glutamine can be modified at a specific site to TAA; a AAG codon specifying lysine can be modified to TAG at a specific site; and a CGA codon for arginine can be modified to a TGA codon at a specific site). Such base pair changes will terminate the reading frame and produce a truncated protein shortened at the site of the stop codon, which truncated protein may be defective or have an altered function. Alternatively, frameshift additions or deletions can be directed at a specific sequence to interrupt the reading frame and produce a garbled downstream protein. Such stop or frameshift mutations can be introduced to determine the effect of knocking out the protein in either plant or animal cells.

Desirable phenotypes that may be obtained in plants by known nucleic acid sequence alterations include, for example, herbicide resistance; male- or female-sterility; salt, drought, lead, freezing and other stress tolerances; altered amino acid content; altered levels or composition of starch; altered levels or composition of oils; and elimination of epitopes in gluten that are known to instigate autoimmune responses in individuals with celiac disease.

The cells within which targeted alterations are effected according to the methods of the present invention can be primary isolated cells, selectively enriched cells, cultured cells, or tissue explants.

In certain ex vivo embodiments of the methods of the present invention, in which targeted sequence alterations are made in non-human cells, such as non-human mammalian ES cells or plant cells, the sequence-altered cells can be used to generate intact organisms, which can thereafter be propagated.

For example, the methods of the present invention can be used to create genetically altered animals, including livestock—such as cattle, bison, horses, goats, sheep, pigs, chickens, geese, ducks, turkeys, pheasant, ostrich and pigeon—to enhance expression of desirable traits, and/or decrease expression of undesirable traits, by first creating genetically altered cells. In other embodiments, the methods of the present invention can be used to create genetically altered animals useful as laboratory models, such as rodents, including mice, rats, guinea pigs; lagomorphs, such as rabbits; monkeys; apes; dogs; and cats. Methods for producing transgenic animals comprising genetically modified cells are known in the art, and are disclosed, for example, in WO 00/51424, “Genetic Modification of Somatic Cells and Uses Thereof,” the disclosure of which is hereby incorporated herein by reference in its entirety.

Further aspects of the present invention are the non-human animals produced thereby.

In other ex vivo embodiments of the methods of the present invention, the targeted sequence alterations are made in human ES cells, which are thereafter used, where legally permissible, to generate tissue or, where permitted, a viable embryo.

In other ex vivo embodiments of the methods of the present invention, in which targeted sequence alterations are made in human non-ES cells, such as hematopoietic progenitor or stem cells, such as CD34⁺CD38⁻ hematopoietic stem cells, the sequence-altered cells can be reintroduced into a human subject for ex vivo gene therapies.

In certain particularly useful embodiments of the methods of the present invention, the first and second oligonucleotides are designed to alter the nucleic acid sequence of an expressed human gene or a plant gene.

The oligonucleotides used in the methods, compositions and kits of the invention can be introduced into cells or tissues by any technique known to one of skill in the art. Such techniques include, for example: electroporation; carrier-mediated delivery using, e.g., liposomes, aqueous-cored lipid vesicles, lipid nanospheres or polycations; naked nucleic acid insertion; particle bombardment and calcium phosphate precipitation. In some embodiments, the oligonucleotides are introduced using electroporation, for example using a BTX ECM® 830 Square Wave electroporator. In other embodiments the transfection is performed with a liposomal transfer compound, for example, DOTAP (N-1-(2,3-Dioleoyloxy) propyl-N,N,N-trimethylammonium methylsulfate, Boehringer-Mannheim) or an equivalent, such as LIPOFECTIN®. In other embodiments, the transfection technique uses cationic lipids. In some embodiments, transfection is performed with Lipofectamine™ 2000 (Invitrogen Corporation, Carlsbad, Calif.).

The methods of the invention can be used with a wide range of concentrations of oligonucleotides. For example, good results can be achieved with 10 nM/10⁵ cells. A ratio of about 500 ng of oligonucleotide in 3 μg of DOTAP per 10⁵ cells can be used. The transfected cells may be cultured in different media, including, for example, in serum-free media, media supplemented with fetal calf serum, human serum albumin, or human serum. The first and second oligonucleotides are typically used in a 1:1 stoichiometric ratio, but other ratios including, e.g., 1:2, 1:3, 1:4 and 1:5, may be used in the methods, composition and kits of the invention. In some embodiments, the first and second oligonucleotides used in the methods and compositions of the invention are administered simultaneously; in other embodiments the oligonucleotides are adjunctively administered.

Further embodiments of the invention are compositions and kits comprising a cell, cell-free extract, or cellular repair protein and at least one oligonucleotide which is capable of effecting a desired sequence alteration at a nucleic acid target site, which sequence alteration confers a selectable phenotype. In some embodiments, the compositions and kits also comprise a second oligonucleotide that is capable of effecting a desired sequence alteration, typically a sequence alteration that is frequently desired and/or is not selectable. In some embodiments the compositions or kits comprise a nucleic acid molecule comprising a nucleic acid sequence which is the target for the at least one oligonucleotide which capable of effecting a desired sequence alteration at a nucleic acid target site, which sequence alteration confers a selectable phenotype.

A cell, cell-free extract, or cellular repair protein for a composition or kit of the invention may be derived from any organism. Compositions and kits of the invention and may comprise any combination of cells, cell-free extracts, or cellular repairs proteins and the cells, cell-free extracts, or cellular repair proteins may be from the same organism or from different organisms. Cellular repair proteins that may be used include, for example, proteins from the RAD52 epistasis group, the mismatch repair group, or the nucleotide excision repair group. In some embodiments, the cell, cell-free extract, or cellular repair protein is or is from a eukaryotic cell or tissue. In some embodiments, the eukaryotic cell is a fungal cell, e.g. a yeast cell. In other embodiments, the cell is a plant cell, e.g., a maize, rice, wheat, barley, soybean, cotton, potato or tomato cell. Other exemplary plant cells include those described elsewhere herein. In some embodiments, the kits comprise a chemical compound selected from the group consisting of: a trichostatin, a histone deacetylase inhibitor and the lambda beta protein. In some embodiments such kits also include instructions for use.

Other embodiments of the invention relate to kits comprising a nucleic acid molecule the nucleic acid sequence of which has been altered according to a method of the invention or using a composition or kit of the invention. In some embodiments, the invention relates to kits comprising a cell comprising a nucleic acid molecule the nucleic acid sequence of which has been altered according to the methods of the invention or using a composition or kit of the invention. In some embodiments, the nucleic acid molecule is selected from the group consisting of: mammalian artificial chromosomes (MACs), PACs from P-1 vectors, yeast artificial chromosomes (YACs), bacterial artificial chromosomes (BACs), plant artificial chromosomes (PLACs), plasmids, viruses or other recombinant vectors.

The purified oligonucleotides compositions may be formulated in accordance with routine procedures as a pharmaceutical composition adapted for bathing cells in culture, for microinjection into cells in culture, and for intravenous administration to human beings or animals. Typically, compositions for cellular administration or for intravenous administration into animals, including humans, are solutions in sterile isotonic aqueous buffer. Where necessary, the composition may also include a solubilizing agent and a local anaesthetic such as lignocaine to ease pain at the site of the injection. Generally, the ingredients will be supplied either separately or mixed together in unit dosage form, for example, as a dry, lyophilized powder or water-free concentrate. The composition may be stored in a hermetically sealed container such as an ampule or sachette indicating the quantity of active agent in activity units. Where the composition is administered by infusion, it can be dispensed with an infusion bottle containing sterile pharmaceutical grade “water for injection” or saline. Where the composition is to be administered by injection, an ampule of sterile water for injection or saline may be provided so that the ingredients may be mixed prior to administration.

Pharmaceutical compositions of this invention comprise the oligonucleotides used in the methods of the present invention and pharmaceutically acceptable salts thereof, with any pharmaceutically acceptable ingredient, excipient, carrier, adjuvant or vehicle.

The oligonucleotides of the invention are preferably administered to the subject in the form of an injectable composition. The composition is preferably administered parenterally, meaning intravenously, intraarterially, intrathecally, interstitially or intracavitarilly. Pharmaceutical compositions of this invention can be administered to mammals including humans in a manner similar to other diagnostic or therapeutic agents. The dosage to be administered, and the mode of administration will depend on a variety of factors including age, weight, sex, condition of the subject and genetic factors, and will ultimately be decided by medical personnel subsequent to experimental determinations of varying dosage as described herein. In general, dosage required for targeted nucleic acid sequence alteration and therapeutic efficacy will range from about 0.001 to 50,000 μg/kg, e.g. between 1 to 250 μg/kg of host cell or body mass or a concentration of between 30 and 60 micromolar.

For cell administration, direct injection into the nucleus, biolistic bombardment, electroporation, liposome transfer and calcium phosphate precipitation may be used. In yeast, lithium acetate or spheroplast transformation may also be used. In one method, the administration is performed with a liposomal transfer compound, e.g., DOTAP (Boehringer-Mannheim), Lipofectamine™ 2000 (Invitrogen™) or an equivalent such as lipofectin. The amount of the oligonucleotide pair used, for example, is about 500 nanograms in 3 micrograms of DOTAP per 100,000 cells or about 1 microgram with 1 microliter Lipofectamine™ 2000 per 1,000,000 cells. For electroporation, between 20 nanograms and 30 micrograms of oligonucleotide per million cells to be electroporated is an appropriate range of dosages which can be increased to improve efficiency of genetic alteration upon review of the appropriate sequence according to the methods described herein.

In order that this invention may be better understood, the following examples are set forth. These examples are for purposes of illustration only, and are not to be construed as limiting the scope of the invention in any manner.

### EXAMPLE 1

### Yeast Cell Targeting Assay Method for Nucleic Acid Sequence Alteration and Preferred Oligonucleotide Selection

In this example, we use single-stranded oligonucleotides to measure oligonucleotide-directed nucleic acid sequence alteration using a Mata wild-type yeast strain with an integrated plasmid with a fusion between a hygromycin resistance gene and eGFP as a target for gene repair (Mata+IntHYG(x)eGFP). Modifications to the oligonucleotides and construction of target vectors are disclosed in WO 01/73002, the disclosure of which is hereby incorporated by reference.

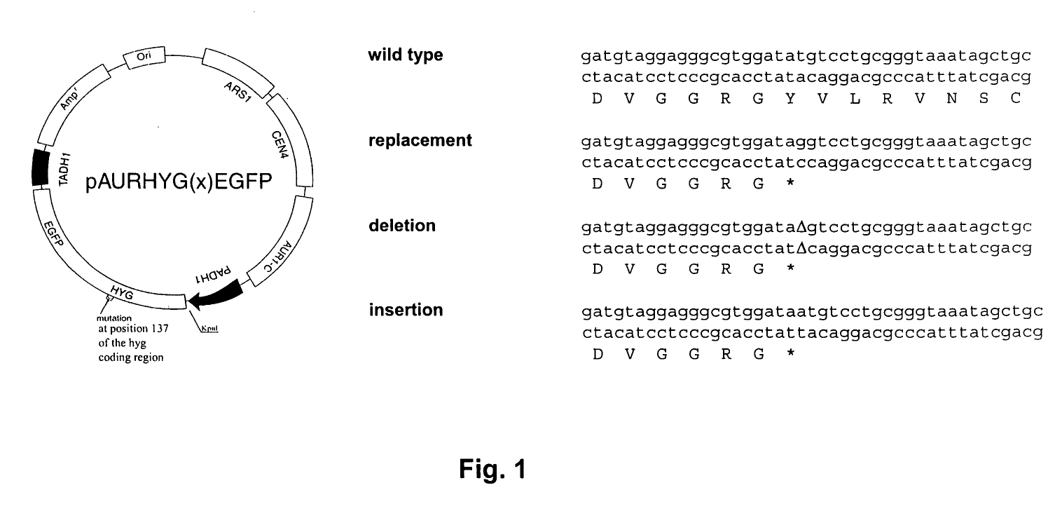

In vivo assay systems. We monitor targeted alteration of genetic material in yeast using both episomal and chromosomal targets. To monitor gene alteration of episomal targets, we employ a yeast system using the plasmids pAURHYG(rep)eGFP, which contains a point mutation in the hygromycin resistance gene, pAURHYG(ins)eGFP, which contains a single-base insertion in the hygromycin resistance gene and pAURHYG(Δ)eGFP which has a single base deletion (shown in FIG. 1). We also use the same plasmid containing a functional copy of the hygromycin-eGFP fusion gene, designated pAURHYG(wt)eGFP, as a control. These plasmids are collectively designated pAURHYG(x)eGFP. These plasmids also contain an aureobasidinA resistance gene. In pAURHYG(rep)eGFP, hygromycin resistance gene function and green fluorescence from the eGFP protein are restored when a G at position 137, in codon 46 of the hygromycin B coding sequence, is converted to a C thus removing a premature stop codon in the hygromycin resistance gene coding region. In pAURHYG(ins)eGFP, hygromycin resistance gene function and green fluorescence from the eGFP protein are restored when an A inserted between nucleotide positions 136 and 137, in codon 46 of the hygromycin B coding sequence, is deleted and a C is substituted for the T at position 137, thus correcting a frameshift mutation and restoring the reading frame of the hygromycin-eGFP fusion gene. In pAURHYG(Δ)eGFP, hygromycin resistance gene function and green fluorescence from eGFP are restored when a C is inserted at the site of the single nucleotide deletion.

We synthesize the set of three yeast expression constructs pAURHYG(rep)eGFP, pAURHYG(Δ)eGFP, pAURHYG(ins)eGFP, that contain a point mutation at nucleotide 137 of the hygromycin-B coding sequence as follows: (rep) indicates a T137G replacement, (Δ) represents a deletion of G137 and (ins) represents an A insertion between nucleotides 136 and 137. We construct this set of plasmids by excising the respective expression cassettes by restriction digest from pHyg(x)eGFP and ligation into pAUR123 (Panvera, Calif.). We digest 10 μg pAUR123 vector DNA as well as 10 μg of each pHyg(x)EGFP construct with Kpnl and Sall (NEB). We gel purify each of the DNA fragments and prepare them for enzymatic ligation. We ligate each mutated insert into pAUR123 vector at a 3:1 molar ratio using T4 DNA ligase (Roche). We screen clones by restriction digest, confirm by Sanger dideoxy chain termination sequencing and purify plasmid DNA using a Qiagen maxiprep kit.

To monitor oligonucleotide-directed alteration of chromosomal nucleic acid sequence targets, we typically employ a yeast system in which we monitor chromosomal genes or we use integrational plasmids such as those designated pAUR101-HYG(x)eGFP. These plasmids do not replicate in yeast. These plasmids comprise the HYG(x)eGFP fusion proteins used in the pAURHYG(x)eGFP episomal plasmid system (shown in FIG. 1) and an aureobasidinA resistance gene. Therefore, like pAURHYG(x)eGFP, these constructs can also be used to monitor all types of gene alterations, i.e. replacements, insertions and deletions. We designate yeast strains into which the pAUR101-HYG(x)eGFP plasmid integrates as “+IntHYG(x)eGFP.” In addition to this construct, we monitor gene alteration of specific yeast genes including, for example, CYC1.

Oligonucleotide synthesis and cells. We synthesize and purify the chimeric, RNA-DNA oligonucleotides with internally duplexed conformation and single-stranded oligonucleotides (including those with the indicated modifications) using available phosphoramidites on controlled pore glass supports. After deprotection and detachment from the solid support, the oligonucleotides are gel-purified using, for example, procedures such as those described in Gamper et al., Biochem. 39, 5808-5816 (2000), or the oligonucleotides are ion-exchange HPLC-purified. We determine the concentration of the oligonucleotides spectrophotometrically (33 or 40 μg/ml per A₂₆₀ unit of single-stranded or hairpin oligonucleotide, respectively). We introduce the oligonucleotides into yeast cells by electroporation as follows: we prepare electrocompetent yeast cells by inoculating 10 ml of YPD media supplemented with 250 μg/ml aureobasidin from a single colony and grow the cultures overnight with shaking at 300 rpm at 30° C. We pellet the cells from the overnight culture by centrifuging at 3000 rpm for 5 minutes, resuspend the cells in 40 ml YPD media (OD₆₀₀ approximately 0.2). We incubate the cells with shaking at 30° C. until the OD₆₀₀ is between 0.5 and 1.0 (3-5 hours). We wash the cells by centrifuging at 4° C. at 3000 rpm for 5 minutes and twice resuspending the cells in 25 ml ice-cold distilled water. We centrifuge at 4° C. at 3000 rpm for 5 minutes and resuspend in 1 ml ice-cold 1M sorbitol and then finally centrifuge the cells at 4° C. at 5000 rpm for 5 minutes and resuspend the cells in 120 μl 1M sorbitol. To transform electrocompetent cells with plasmids or oligonucleotides, we mix 40 μl of cells with oligonucleotide (typically 5 μg or amounts as indicated) and incubate on ice for 5 minutes. We transfer the mixture to a 0.2 cm electroporation cuvette and electroporate with a BIO-RAD Gene Pulser apparatus at 1.5 kV, 25 μF, 200 Ω for one five-second pulse. We then immediately resuspend the cells in 3 ml YPD media supplemented with 2M sorbitol and incubate the cultures at 30° C. with shaking at 300 rpm for 6 hours. We spread 200 μl of this culture on selective plates containing 300 μg/ml hygromycin and spread 200 μl of a 10⁵ dilution of this culture on selective plates containing 500 ng/ml aureobasidinA and/or and incubate at 30° C. for 3 days to allow individual yeast colonies to grow. We count the colonies on the plates and calculate the gene conversion efficiency by determining the number of hygromycin resistance colonies per 10⁵ aureobasidinA resistant colonies.

Oligonucleotides direct gene alteration. We use this system to assay the ability of various oligonucleotides (sequences shown in Table 1) to support correction under a variety of conditions. The oligonucleotides are designed so that they can direct correction of the replacement, insertion and deletion mutations in Mata+IntHYG(x)eGFP. The oligonucleotides generally are centered around the base targeted for alteration. In this example, we test the ability of these oligonucleotides to direct alteration of nucleic acid sequence in Mata+IntHYG(rep)eGFP (see Table 2).

We also use additional oligonucleotides to assay the ability of individual oligonucleotides to correct multiple mutations in both the pAURHYG(x)eGFP plasmid and in yeast strains with integrated copies of pAUR101-HYG(x)eGFP. These include, for example, an oligonucleotide that alters two basepairs that are 3 nucleotides apart with the sequence 5′-CTC GTG CTT TCA GCT TCG ATG TAG GAG GGC GTG GGT ACG TCC TGC GGG TM ATA GCT GCG CCG ATG GTT TCT AC-3′ (SEQ ID NO: _); a 74-mer that alters two basepairs that are 15 nucleotides apart with the sequence 5′-CTC GTG CTT TCA GCT TCG ATG TAG GAG GGC GTG GAT ACG TCC TGC GGG TAA ACA GCT GCG CCG ATG GTT TCT AC-3′ (SEQ ID NO: _); and a 74-mer that alters two basepairs that are 27 nucleotides apart with the sequence 5′-CTC GTG CTT TCA GCT TCG ATG TAG GAG GGC GTG GAT ACG TCC TGC GGG TM ATA GCT GCG CCG ACG GTT TCT AC (SEQ ID NO: _). The nucleotides in these oligonucleotides that direct alteration of the target sequence are in boldface. These oligonucleotides are chemically modified to enable them to effect oligonucleotide-directed nucleic acid sequence alteration.

TABLE 1
Oligonucleotides used in Examples 1 and 2

| | | | |
|---|---|---|---|
| Name | Size | Sequence | SEQ ID NO: |

| | | |
|---|---|---|
| Hyg3S/74T | 74 mer | 5′-C*T*C* GTG CTT TCA GCT TCG ATG TAG GAG |
| | | GGC GTG GAT ACG TCC TGC GGG TAA ATA GCT |
| | | GCG CCG ATG GTT TC *T*A*C-3′ |
| Hyg3S/74NT | 74 mer | 5′-G*T*A* GAA ACC ATC GGC GCA GCT ATT TAC |
| | | CCG CAG GAC GTA TCC ACG CCC TCC TAC ATC |
| | | GAA GCT GAA AGC AC*G *A*G-3′ |

Phosphorothioate linkages are indicated as “*” between the bases. The base corresponding to the location of the replacement mutation in the Hyg(rep) target is in bold.

TABLE 2
Gene alteration in Mata+IntHYG(rep)eGFP

| Oligonucleotide | Alteration per 10⁵ | Fold correction (relative to |
|---|---|---|
| assayed | Aureobasidin^{R} colonies¹ | Hyg3S/74T) |
| Hyg3S/74T | 0.22 ± 0.3 | 1x |
| Hyg3S/74NT | 0.89 ± 1.3 | 4.0x |

¹These numbers represent an average of four experiments with the standard deviation indicated for alteration efficiency.

### EXAMPLE 2

### Selection for a First Alteration Reduces Screening Required to Identify a Second Alteration in the Same Cell

In this example we demonstrate that the efficiency of alteration of nucleic acid sequence at a second site is enhanced in a population of nucleic acid molecules that have been previously selected for alteration at a first target site. We use yeast strains having an integrated copy of the pAUR101-HYG(rep)eGFP plasmid described in Example 1 that also contain the βS YAC (230 kb YAC comprising the human β-globin gene). In some experiments, the yeast strains also contain a plasmid that overexpresses yeast Rad51, designated pYNARad51. We use several oligonucleotides: Hyg3S/74NT, which, as described in Example 1, is capable of directing alteration of the mutated Hyg(x)eGFP target to confer hygromycin resistance; and βS-386m and βS-378m, each of which is a 71-mer oligonucleotide with 3 phosphorothioate linkages on each end, which is capable of directing a mutation in the human β-globin gene. The sequence of βS-386m is 5′-G*C*C* TCA CCA CCA ACT TCA TCC ACG TTC ACC TTG CCT CAC AGG GCA GTA ACG GCA GAC TTC TCC ACA GG*A *G*T-3′ (SEQ ID NO: _) and the sequence of βS-378m is 5′-T*A*A* CGG CAG ACT TCT CCA CAG GAG TCA GGT GCA CCG TGG TGT CTG TTT GAG GTT GCT AGT GAA CAC AG*T *T*G-3′ (SEQ ID NO: _). βS-386m and βS-378m both hybridize to the non-transcribed sequence of the human β-globin gene and direct a nucleic acid sequence alteration that creates a β-thalassemia mutation: βS-386m converts a TGG codon to a stop codon (TGA) and βS-378m converts the ATG start codon to ACG.

We introduce the oligonucleotides into the yeast cells by electroporation as follows: we prepare electrocompetent yeast cells by inoculating a single colony into 10 ml of appropriate media supplemented with 250 μg/ml aureobasidin and grow the cultures overnight with shaking at 300 rpm at 30° C. In this Example, we use YPD media unless the cells contain a plasmid to overexpress a repair protein when we use SC-ade media. We dilute the cells into 40 ml media to an initial OD₆₀₀ of approximately 0.15. We incubate the cells with shaking at 30° C. until the OD₆₀₀ is approximately 0.25 and, in some experiments, we add 100 mM hydroxyurea (HU). We continue to incubate the cells with shaking at 30° C. until the OD₆₀₀ is approximately 0.6. We spin down the cells at 3000 rpm for 5 minutes and resuspend in 1 ml YPD supplemented with 25 μl 1M DTT and incubate the culture with shaking at 30° C. for 20 minutes. We wash the cells four times by centrifuging at 4° C. at 3000 rpm for 5 minutes and resuspending the cells twice in 25 ml ice-cold distilled water; once in 25 ml ice-cold 1M sorbitol; and once in 1 ml ice-cold 1M sorbitol. We centrifuge the cells at 4° C. at 5000 rpm for 5 minutes and resuspend the cells in 120 μl 1M sorbitol. We transform the electrocompetent cells with Hyg3S/74NT and either βS-386m or βS-378m by mixing 40 μl of cells with 30 μg oligonucleotide (or 30 μg of each oligonucleotide) and incubate on ice for 5 minutes. We transfer the mixture to a 0.2 cm electroporation cuvette and electroporate with a BIO-RAD® Gene Pulser® apparatus at 1.5 kV, 25 μF, 200 Ω for one approximately five-second pulse. We then resuspend the cells in 3 ml YPD media supplemented with Aureobasidin and allow the cells to recover by incubating them overnight at 30° C. with shaking at 300 rpm. In some experiments, we supplement the culture with 50 mg/ml Trichostatin A (TSA) during this recovery phase. We spin down the cells, resuspend in 1 ml YPD and spread 100 μl of this culture (and/or 100 μl of 10¹ or 10² dilutions) on selective plates containing 300 μg/ml hygromycin and spread 200 μl of a 10⁵ dilution of this culture on selective plates containing 500 ng/ml aureobasidinA and incubate at 30° C. for 3 days to allow individual yeast colonies to grow. We count the colonies on the plates and calculate the conversion efficiency for the hygromycin target by determining the number of hygromycin resistance colonies per 10⁵ aureobasidinA resistant colonies. We pick individual colonies from YPD-hygromycin plates into 96-well plates with 150 μl YPD/well and allow the cells to grow overnight at 30° C. We screen individual wells for alteration of the β-globin target in the βS YAC as follows: we PCR amplify a 345 bp fragment of the β-globin gene using forward primer PCO2; 5′-TCC TAA GCC AGT GCC AGA AGA-3′ (SEQ ID NO: _) and reverse primer PCO5; 5′-CTA TTG GTC TCC TTA MC CTG-3′ (SEQ ID NO: _) and purify the PCR product. We analyze the sequence at the target nucleotide in the PCR products by SNaPshot™ analysis on an ABI3100 (Applied Biosystems™) using primers corresponding to the different alterations directed by βS-386m and βS-378m as follows: 386RC; 5′-CCC CCC CCC CCC CCC CCA AGT CTG CCG TTA CTG CCC TGT G-3′ (SEQ ID NO: _) and 378MF; 5′-TTT TTT TTT TTT TTT TTT TTT TTT TTT TTT TTT TGC AAC CTC AAA CAG ACA CCA-3′ (SEQ ID NO: _).

As shown in Tables 3 and 4, we observe that the alteration efficiency of the β-globin gene is approximately 10-fold higher than for alteration of the hygromycin target. We observe this result with both βS-386m and βS-378m as well as in the presence or absence of additional factors that enhance oligonucleotide-directed nucleic acid sequence alteration such as, e.g., overexpression of Rad51.

In other experiments, we do the experiments in yeast strains overexpressing other repair protein(s) in place of or in addition to Rad51and/or yeast strains with one or more mutations in repair proteins. We also do experiments where we add other compounds that enhance oligonucleotide-directed nucleic acid sequence alteration such as histone deacetylase inhibitors and the bacteriophage lambda beta protein.

TABLE 3
βS-386m Experiment

| | | Hygromycin | β-globin |
|---|---|---|---|
| | | Alteration | Alteration |
| Yeast Strain and treatment | Oligonucleotide(s) | Efficiency (/10⁵) | Efficiency |
| Mata−intHyg(rep)eGFP/βYAC (HU + TSA) | Hyg3S/74NT | 12.35 | NA |
| Mata−intHyg(rep)eGFP/βYAC (HU + TSA) | Hyg3S/74NT + βS-386m | 30.51 | 0.0031 |
| Mata−intHyg(rep)eGFP/βYAC | Hyg3S/74NT | 3.22 | NA |
| Mata−intHyg(rep)eGFP/βYAC | Hyg3S/74NT + βS-386m | 5.07 | ND |

NA = not applicable;
ND = not determined

TABLE 4
βS-378m Experiment

| | | Hygromycin | β-globin |
|---|---|---|---|
| | | Alteration | Alteration |
| Yeast Strain and treatment | Oligonucleotide(s) | Efficiency (/10⁵) | Efficiency |
| Mata−intHyg(rep)eGFP/βYAC | Hyg3S/74NT + βS-378m | 44.58 | ND |
| (HU + TSA) | | | |
| Mata−intHyg(rep)eGFP/βYAC | Hyg3S/74NT | 12.79 | NA |
| (HU + TSA) | | | |
| Mata−intHyg(rep)eGFP/βYAC | KanUD3/71 | 0.00 | NA |
| (HU + TSA) | | | |
| Mata−intHyg(rep)eGFP/βYAC + pYNARad51 | Hyg3S/74NT + βS-378m | 123.73 | 0.15 |
| (HU + TSA) | | | |
| Mata−intHyg(rep)eGFP/βYAC + pYNARad51 | Hyg3S/74NT | 96.96 | NA |
| (HU + TSA) | | | |
| Mata−intHyg(rep)eGFP/βYAC + pYNARad51 | KanUD3/71 | 0.01 | NA |
| (HU + TSA) | | | |

NA = not applicable;
ND = not determined;
KanUD3/71 is a negative control oligonucleotide

### EXAMPLE 3

### Selection for a First Alteration Reduces Screening Required to Identify a Second Alteration in Human Blood Cells

Assay system. We monitor targeted alteration of genetic material in human blood cells using the chromosomal gene encoding the beta subunit of hemoglobin as the target. We cointroduce two oligonucleotides with a plasmid comprising a mutant copy of the green fluorescent protein (GFP) gene. The second oligonucleotide is designed to direct an alteration which repairs the mutant GFP resulting in fluorescence. The first oligonucleotide is designed to convert the wild-type allele to the sickle allele. We use first oligonucleotides that correspond in sequence to the wild-type allele at all positions except the single nucleotide position designed to introduce the sickle mutation into the gene. Therefore, these oligonucleotides are identical to the oligonucleotides described in Example 6 and shown in Table 7 except for a single base. For example, we use first oligonucleotides selected from: 5′-C*A*A* CCT CAA ACA GAC ACC ATG GTG CAC CTG ACT CCT GtG GAG MG TCT GCC GTT ACT GCC CTG TGG GGC AA*G *G*T-3′; SEQ ID NO: _; 5′-A*C*C* TTG CCC CAC AGG GCA GTA ACG GCA GAC TTC TCC aCA GGA GTC AGG TGC ACC ATG GTG TCT GTT TGA GG*T *T*G-3′; SEQ ID NO: _; 5′-ACC TCA AAC AGA CAC CAT GGT GCA CCT GAC TCC TGt GGA GAA GTC TGC CGT TAC TGC CCT GTG GGG CAA GG -3′; SEQ ID NO: _; 5′-G*A*C* ACC ATG GTG CAC CTG ACT CCT GtG GAG AAG TCT GCC GTT ACT GCC *C*T*G-3′; SEQ ID NO: _; and 5′-A*C*C* TCA AAC AGA CAC CAT GGT GCA CCT GAC TCC TGt GGA GAA GTC TGC CGT TAC TGC CCT GTG GGG CA*A *G*G -3′. The bases in the oligonucleotides which are mismatched to the wild-type allele are shown in lowercase. The oligonucleotides are synthesized with three phosphorothioate linkages on each end (represented with asterisks) or with a single LNA base at each end (bold).

Preparation and treatment of cells. We thaw and electroporate cells as follows. We warm QBSF-60 medium (Quality Bio) containing 10% FCS (StemCell Technologies) to 37° C. We quickly thaw frozen G-CSF mobilized peripheral blood CD-34+cells (BioWhittaker) in a 37° C. water bath, wipe the outside of the tube with 70% ethanol and aseptically transfer about 2 ml (approximately 1×10⁶ cells) of cell suspension to a 15 ml or 50 ml conical tube. We rinse the vial in 1 ml of medium and add it dropwise to the cells, gently swirling the conical tube every few drops. We slowly add medium dropwise until the volume is about 5 ml, still gently swirling the conical tube every few drops, and then slowly bring the volume up to fill the tube by adding 1-2 ml of medium dropwise, swirling after every addition. We centrifuge the cell suspension at 200×g (1500 rpm) for 15 minutes at room temperature. We use a pipet to remove most of the wash to a second tube, leaving a few ml behind to avoid disturbing the cell pellet. We resuspend the pellet in the remaining medium and transfer to a 15 ml conical tube. We rinse the original tube with 5 ml medium and add the wash to the cells dropwise, swirling gently after each addition, and recentrifuge at 200×g for 15 minutes. We pipet off all but 2 ml of the wash and gently resuspend the cells in the remaining medium and count them. We rest the cells at 37° C. and 5% CO₂ for 1 hour and then recount the cells. We add 5 ml QBSF-60 medium without FCS containing the cytokines fit-3, SCF and TPO at 100 ng/ml final concentration (Stem Cell Technologies), repellet the cells at 200×g (1500 rpm for 15 min), and gently remove as much liquid volume as possible without disturbing the pellet. We resuspend the cells at about 5×10⁵-1×10⁶ cells/ml and transfer them to 6-well tissue culture treated dishes. We stimulate the cells for three days with cytokines (QBSF-60 medium without FCS containing the cytokines flt-3, SCF and TPO at 100 ng/ml final concentration) and perform a cell count using trypan blue exclusion staining. We centrifuge the cells at 200×g (1500 rpm) for 15 minutes. We remove the excess volume by pipet and resuspend the cells in the same medium at 2×10⁶ cells/ml.

We electroporate the oligonucleotides and the GFP plasmid into the cells under square wave conditions as follows. We add 250 μl cell suspension, 5 μg GFP plasmid and 30 μg each oligonucleotide to a 2 mm gap cuvette and electroporate for one 19 msec pulse at 220 V. We then add 750 μl Iscove's Medium (Invitrogen™), 10% FCS (StemCell Technologies) and the cytokines flt-3, SCF, and TPO at 100 ng/ml final concentration, glutamine and penicillin/streptomycin. Alternatively, we add 250 μl cell suspension, 250 μl QBSF-60 medium supplemented with flt-3, SCF and TPO and 30 μg oligonucleotide to a 4 mm gap cuvette and electroporate for five 19 msec pulse at 220 V with a pulse interval of 1 sec. We then add 500 μl Iscove's Medium (Invitrogen™), 10% FCS (StemCell Technologies) and the cytokines flt-3, SCF and TPO at 100 ng/ml final concentration. We select for the repair of the mutant GFP protein using by FACS and analyze the sequence of the hemoglobin target by PCR amplification and analysis on the SNapShot™ device using two oligonucleotides: 5′-TTT TTT TTT TTT TTT GAC ACC ATG GTG CAC CTG ACT CCT G -3′; SEQ ID NO _; and 5′-TTT TTT TTT TTT TTT TTT TTC AGT AAC GGC AGA CTT CTC C-3′; SEQ ID N _. As we see in other cells, these oligonucleotides direct targeted alteration in human blood cells.

### EXAMPLE 4

### Use of HU and TSA in Dual Targeting Experiments

The efficiency of targeted alteration can be increased and the cost decreased by using at least two unrelated oligonucleotides simultaneously in dual targeting experiments. In this approach, alteration by a first oligonucleotide confers a selectable phenotype that is selected for. Alterations directed by a second oligonucleotide are then screened for from within this selected population. Because the population identified by selective pressure is enriched for cells that bear an edited base at the non-selective site, the approach is useful as a method, termed gene editing, for rapidly and efficiently introducing a single nucleotide polymorphism of choice into virtually any gene at any desired location using modified single-stranded oligonucleotides.

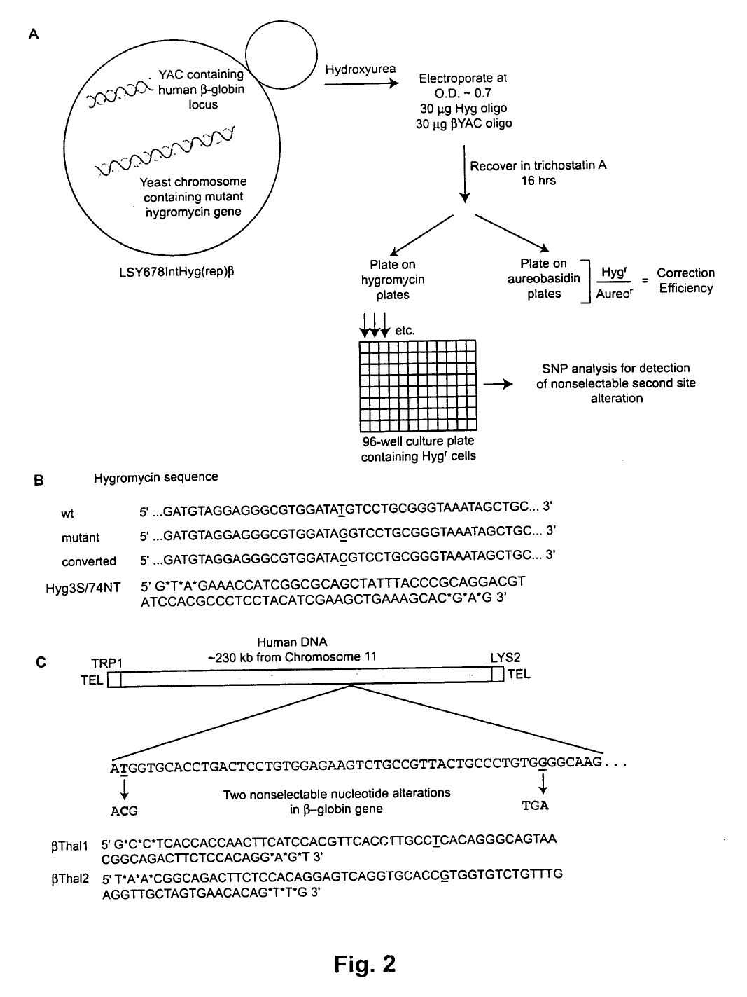

The dual targeting strategy is illustrated in FIG. 2A. The LSY678intHyg(rep)βstrain (Table 5) contains a 240 kb human β^{s}-globin YAC and a cassette containing a chromosomal hygromycin-resistance gene inactivated by a single base mutation and a functional aureobasidin-resistance gene. See Liu et al., Nucleic Acids Res. 31:2742-2750 (2002); Parekh-Olmedo et al., Chem. Biol. 9:1073-1084 (2002); and Liu et al., Mol. Cell Biol. 22:3852-3863 (2002). FIG. 2B shows the oligonucleotide that is used to direct editing of the chromosomal hygromycin mutant gene. Hyg3S/74NT (SEQ ID NO: _) is a 74-mer that is specific for inding to the nontranscribed strand and contains three terminal phosphorothioate linkages. Id. Also shown is the target sequence of the mutant, which contains a TAG stop codon. FIG. 2C illustrates the structure of the β-globin YAC and nucleotides targeted for editing are specified. The two nonselectable changes are directed by different oligonucleotides, βThal1 (SEQ ID NO: _) and βThal2 (SEQ ID NO: _), in separate experiments. The YAC contains approximately 230 kb of genomic DNA from human chromosome 11, indicated by the shaded region. The unshaded regions represent the yeast sequences that are on either end of the YAC (not drawn to scale). Yu et al., Proc. Natl. Acad. Sci. USA 97:5978-5983 (2000). A portion of the β-globin sequence is shown, beginning with the start codon. βThal1 directs a change from a G to an A while βThal2 directs a change from a T to a C. The sequences of the oligonucleotides having nucleic acid sequence alteration activity are shown and are designed to bind to the non-transcribed strand, relative to human transcription of the β-globin locus. Both changes result in single-base substitutions that have been documented to result in β-thalassemia in humans.

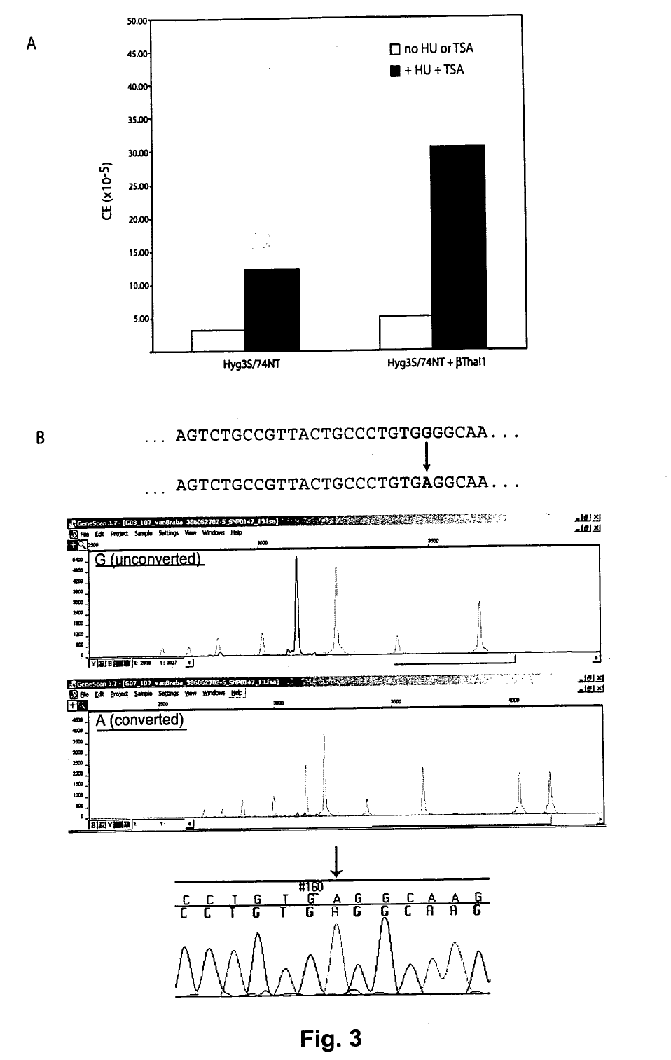

For editing experiments, YAC-containing LSY678IntHyg(rep)βcells (Table 5) are grown in the presence of HU, electroporated with the selectable and nonselectable oligonucleotides, and allowed to recover in the presence of TSA (FIG. 2A). Because the human β-globin gene is likely to be transcriptionally inactive in yeast, HU and TSA are especially important in increasing target accessibility. The results of dual targeting experiments are presented in FIG. 3A. Hygromycin-resistant colonies are observed when the oligonucleotide, Hyg3S/74NT, is used. The ratio of hygromycin-resistant colonies to aureobasidin-resistant colonies is referred to as the correction efficiency (C.E.). The presence of HU and TSA leads to an increase in the C.E. of the hygromycin mutation, here about 4- to 6-fold. In this experiment, hygromycin-resistant colonies are found at roughly 1 per 3000 aureobasidin-resistant colonies. Hygromycin-resistant colonies are then analyzed for second-site editing in the YAC β-globin gene. The βThal1 oligonucleotide is designed to direct the replacement of a G in TGG codon 16 of exon 1 with an A, giving the stop codon TGA (FIG. 2C). FIG. 3B shows an ABI SNaPshot (middle panels) and direct DNA sequence (bottom panel) of a region of the b-globin gene in a corrected colony from this experiment; in both, the G to A change is evident. Of those colonies that are corrected in the hygromycin mutation, 1 in 325 also contain the second change in the YAC β-globin sequence. Thus, approximately 10% of the cells with the corrected hygromycin-resistance gene also contain the edited β-globin gene.

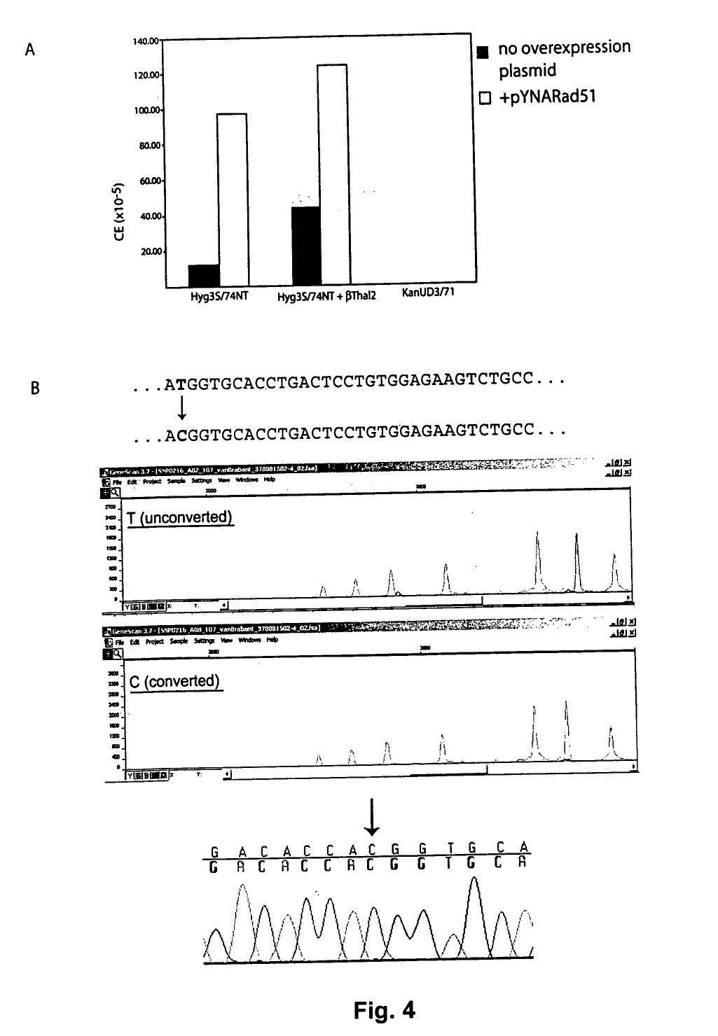

As shown in various experiments above, overexpression of RAD51 consistently increases the frequency of chromosomal gene editing. Accordingly, we introduce an expression plasmid containing the yeast RAD51 gene into LSY678IntHyg(rep)β cells (Table 5). FIG. 4 shows results of dual targeting in this strain and, as expected, expression of RAD51 increases the hygromycin correction efficiency of oligonucleotide Hyg3S/74NT (compare with FIG. 3). For these editing experiments, YAC-containing LSY678IntHyg(rep)β cells (Table 5) are grown in the presence of HU, electroporated with the selectable and nonselectable oligonucleotides, and allowed to recover in the presence of TSA (FIG. 2A). Here too, addition of a second oligonucleotide, βThal2, increases the correction efficiency further, to roughly 1 hygromycin-resistant colony per 800 aureobasidin-resistant colonies.

The βThal2 oligonucleotide is designed to direct the replacement of a T in the initiator ATG codon of exon 1 with a C, giving the non-initiator codon ACG (FIG. 2). FIG. 4B shows an ABI SNaPshot (middle panels) and direct DNA sequence (bottom panel) of the β-globin gene from a corrected Hyg^{r} colony; the T to C change is evident in both analytical panels. Importantly, of those colonies that are corrected in the hygromycin mutation, 1 in 70 also contain the second single-base change in the YAC β-globin sequence. Thus, the dual targeting approach is again successful; approximately 10% of the cells bearing the corrected hygromycin also contain the edited β-globin gene. In addition, in the presence of high levels of Rad51, gene editing occurs at a higher level, indicating that the presence of HU, TSA, and RAD51 overexpression exhibit synergistic effects on the overall process.

TABLE 5
Genotype of yeast strains

| Strain | Genotype/Description |
|---|---|
| AB1380 | MATa ura3 trp1 ade2-1 can1-100 lys2-1 his5 ψ+ |
| LSY678 | MATa ura3 trp1-1 ade2-1 leu2-3, 112 can1 his3-11, 15 |
| LSY678IntHyg(rep) | LSY678 with mutant hygromycin gene and functional |
| | aureobasidin-resistance gene integrated into the AUR-1 |
| | locus on chromosome XI |
| LSY678IntHyg(rep)β | LSY678IntHyg(rep) with 250 kb YAC containing the human |
| | β-globin locus |
| LSY678IntHyg(rep)β + pYNARAD51 | The above strain containing an episomal expression |
| | plasmid overexpressing RAD51 |

Strains. The genotypes of the yeast strains used in these studies are listed in Table 5. Details of the LSY678IntHyg(rep) strain are published in Liu et al., Mol. Cell Biol. 22:3852-3863 (2002).

YAC Manipulations. The β-globin YAC is isolated from a preparative pulsed-field gel as described in Gnirke et al., Genomics 15:659-667 (1993). Briefly, concentrated chromosomal DNA from the βS-YAC strain (AB1380 background, see Chang et al., Proc. Natl. Acad. Sci. USA 95:14886-14890 (1998)) is prepared and resolved on a 1% low-melt agarose pulsed-field gel at 200V, 14° C., 20-50 s, 33 hours. The YAC is isolated, equilibrated with a modified agarase buffer (10 mM BisTris-HCl pH6.5, 1 mM EDTA, 100 mM NaCl), treated with β-agarase I (New England Biolabs), and concentrated to a final volume of ˜200 μl. Thirty βl of the purified YAC are introduced into competent LSY678IntHyg(rep) cells by spheroplast transformation and selection on agar/sorbitol plates lacking tryptophan. Transformants are restreaked and confirmed by pulsed-field gel electrophoresis, PCR, and sequence analysis for a fragment of the human β-globin gene.

The pYNARad51 episomal expression plasmid is constructed by replacing the TRP1 gene of pYNRad51 (see Liu et al., Nucleic Acids Res. 31, 2742-2750 (2002)) with the ADE2 gene. pYNARad51 is introduced into LSY678IntHyg(rep)β by electroporation and selection on agar plates lacking adenine.

Oligonucleotides. Hyg3S/74NT (SEQ ID NO: _), βThal1 (SEQ ID NO: _), and βThal2 (SEQ ID NO: _) are ordered from IDT with HPLC purification. Hyg3S/74NT is a 74mer and both βThal1 and βThal2 are 71mers; all three oligonucleotides have three phosphorothioate linkages at the 5′ and 3′ ends (FIG. 2).

Dual Targeting. The dual targeting protocol is outlined in FIG. 2A. LSY678IntHyg(rep)β cells are grown overnight in 10 ml YPD media at 30° C. The culture is diluted to OD₆₀₀ ˜0.15-0.20 in 40 ml YPD media and grown for one doubling time to OD₆₀₀ ˜0.3-0.4. 100 mM HU is added to the culture and the cells are grown for one doubling time to OD_{600 ˜0.6}-0.8. Cells are harvested and resuspended in 1 ml YPD containing 25 μl 1M DTT and grown for an additional 20 minutes at 30° C. The cells are washed twice with 25 ml cold dH₂0 and once with 25 ml cold 1M sorbitol. The cells are resuspended gently in 1 ml cold 1M sorbitol, spun for 5 minutes at 5000 rpm in a microcentrifuge, and resuspended in 120 μl 1M sorbitol. Forty microliters of cells are electroporated with 30 μg of each oligonucleotide in a 2 mm gap cuvette using a Bio-Rad Gene Pulser apparatus (Richmond, Calif.) with 1.5 kV, 25 μF, 200Ω, 1 pulse, 5 s/pulsed length. The cells are immediately resuspended in 3 ml YPD with 0.8 μg/ml aureobasidin A and 50 μg/ml TSA and recovered overnight at 30° C. The cells are spun down and resuspended in 1 ml fresh YPD. Dilutions are plated on YPD agar plates containing either hygromycin (300 μg/ml) or aureobasidin A (0.5 μg/ml). Correction efficiencies (C.E.s) are determined based on the number of hygromycin-resistant colonies per aureobasidin-resistant colonies.

Individual colonies are picked from the hygromycin agar plates into 96-well plates (Corning) containing 150 μl YPD and grown overnight at 30° C. with shaking. A 345 bp PCR product specific for the human β-globin locus is amplified from each of the 96 wells using the primers PCO2 (5′-TCCTAAGCCAGTGCCAGAAG-3′ (SEQ ID NO.: _)) and PCO5 (5′-CTATTGGTCTCCTTAAACCTG-3′ (SEQ ID NO.: _)) in order to screen for the βThal1 or βThal2 conversion. The PCR reactions are performed by adding 8 pmoles of each primer and 2.5 μl yeast cell culture into pre-aliquoted PCR reaction mixes (Marsh/Abgene). The PCR reactions use an annealing temperature of 45.8° C. and an extension time of 1 min for 35 cycles. The PCR reactions are purified using a QiaQuick PCR 96-well purification kit (Qiagen) and eluted in a volume of 80 μl. One microliter of the purified PCR product is used as a template for the ABI SNaPshot reaction. The sequence of the SNaPshot primer used to screen for the βThal1 conversion is: 5′-CCCCCCCCCCCCCCCCCAAGTCTGCCGTTACTGCCCTGTG-3′ (SEQ ID NO: _). The sequence of the SNaPshot primer used to screen for the βThal2 conversion is: 5′-TTTTTTTTTTTTTTTTTTTTTTTTTTTTTTTTTTCCACAGGAGTCAGGTGCACC-3′ (SEQ ID NO: _). The SNaPshot reactions are performed using an ABI Prism SNaPshot Multiplex Kit, as specified by the manufacturer, and analyzed on an ABI 3100 Genetic Analyzer.

Sequence Analysis. Any potential converted clones from the SNaPshot reactions are confirmed by sequence analysis. Both strands of the PCR products are sequenced using primers PCO2 and PCO5 by Sanger dideoxy sequencing using an ABI Prism kit, as specified by the manufacturer, on an automated ABI 3100 Genetic Analyzer.

### EXAMPLE 5

### Adenosine Deaminase (ADA)

Adenosine deaminase (ADA, EC 3.5.4.4) catalyses the deamination of adenosine and 2′-deoxyadenosine to inosine or 2′-deoxyinosine respectively. ADA deficiency has been identified as the metabolic basis for 20-30% of cases with recessively inherited severe combined immunodeficiency (SCID). Affected infants are subject to recurrent chronic viral, fungal, protozoal, and bacterial infections and frequently present with persistent diarrhea, failure to thrive and candidiasis. In patients homozygous for ADA deficiency, 2′-deoxyadenosine accumulating during the rapid turnover of cells rich in DNA is converted back to dATP, either by adenosine kinase or deoxycytidine kinase. Many hypotheses have been advanced to explain the specific toxicity to the immune system in ADA deficiency. The apparently selective accumulation of dATP in thymocytes and peripheral blood B cells, with resultant inhibition of ribonucleotide reductase and DNA synthesis is probably the principal mechanism.

The structural gene for ADA is encoded as a single 32 kb locus containing 12 exons. Studies of the molecular defect in ADA-deficient patients have shown that mRNA is usually detectable in normal or supranormal amounts. Specific base substitution mutations have been detected in the majority of cases with the complete deficiency. A C-to-T base substitution mutation in exon 11 accounts for a high proportion of these, whilst a few patients are homozygous for large deletions encompassing exon 1. A common point mutation resulting in a heat-labile ADA has been characterised in some patients with partial ADA deficiency, a disorder with an apparently increased prevalence in the Caribbean.

As yet no totally effective therapy for ADA deficiency has been reported, except in those few cases where bone marrow from an HLA/MLR compatible sibling donor was available.

Two therapeutic approaches have provided long-term benefit in specific instances. First, reconstitution using T cell depleted mismatched sibling marrow has been encouraging, particularly in early presenters completely deficient in ADA. Secondly, therapy with polyethylene glycol-modified adenosine deaminase (PEG-ADA) for more than 5 years has produced a sustained increase in lymphocyte numbers and mitogen responses together with evidence of in vivo B cell function. Success has generally been achieved in late presenters with residual ADA activity in mononuclear cells.

ADA deficiency has been chosen as the candidate disease for gene replacement therapy and the first human experiment commenced in 1990. The clinical consequences of overexpression of ADA activity—one of the potential hazards of gene implant—are known and take the form of an hereditary haemolytic anaemia associated with a tissue-specific increase in ADA activity. The genetic basis for the latter autosomal dominant disorder seemingly relates to markedly increased levels of structurally normal ADA mRNA.

TABLE 6
ADA Mutations and Genome-Correcting Oligos

| | | |
|---|---|---|
| Clinical Phenotype & | | SEQ ID |
| Mutation | Correcting Oligos | NO: |

| | | |
|---|---|---|
| Adenosine deaminase | AGAGACCCACCGAGCGGCGGCGGAGGGAGCAGCGCCGGGG | 1 |
| deficiency | CGCACGAGGGCACCATGGCCCAGACGCCCGCCTTCGACAAG | |
| GLN3TERM | CCCAAAGTGAGCGCGCGCGGGGGCTCCGGGGACGGGGGTC | |
| CAG to TAG | GACCCCCGTCCCCGGAGCCCCCGCGCGCGCTCACTTTGGGC | 2 |
| | TTGTCGAAGGCGGGCGTCTGGGCCATGGTGCCCTCGTGCGC | |
| | CCCGGCGCTGCTCCCTCCGCCGCCGCTCGGTGGGTCTCT | |
| | CCATGGCCCAGACGCCC | 3 |
| | GGGCGTCTGGGCCATGG | 4 |
| Adenosine deaminase | TATTTGTTCTCTCTCTCCCTTTCTCTCTCTCTTCCCCCTGCCCC | 5 |
| deficiency | CTTGCAGGTAGAACTGCATGTCCACCTAGACGGATCCATCAA | |
| HIS15ASP | GCCTGAAACCATCTTATACTATGGCAGGTAAGTCC | |
| CAT to GAT | GGACTTACCTGCCATAGTATAAGATGGTTTCAGGCTTGATGGA | 6 |
| | TCCGTCTAGGTGGACATGCAGTTCTACCTGCAAGGGGGCAG | |
| | GGGGAAGAGAGAGAGAAAGGGAGAGAGAGAACAAATA | |
| | TAGAACTGCATGTCCAC | 7 |
| | GTGGACATGCAGTTCTA | 8 |
| Adenosine deaminase | TCCCTTTCTCTCTCTCTTCCCCCTGCCCCCTTGCAGGTAGAAC | 9 |
| deficiency | TGCATGTCCACCTAGACGGATCCATCAAGCCTGAAACCATCTT | |
| GLY20ARG | ATACTATGGCAGGTAAGTCCATACAGAAGAGCCCT | |
| GGA to AGA | AGGGCTCTTCTGTATGGACTTACCTGCCATAGTATAAGATGGT | 10 |
| | TTCAGGCTTGATGGATCCGTCTAGGTGGACATGCAGTTCTAC | |
| | CTGCAAGGGGGCAGGGGGAAGAGAGAGAGAAAGGGA | |
| | ACCTAGACGGATCCATC | 11 |
| | GATGGATCCGTCTAGGT | 12 |
| Adenosine deaminase | CCTGGAGCTCCCAAGGGACTTGGGGAAGGTTGTTCCCAACC | 13 |
| deficiency | CCTTTCTTCCCTTCCCAGGGGCTGCCGGGAGGCTATCAAAAG | |
| GLY74CYS | GATCGCCTATGAGTTTGTAGAGATGAAGGCCAAAGAGG | |
| GGC to TGC | CCTCTTTGGCCTTCATCTCTACAAACTCATAGGCGATCCTTTT | 14 |
| | GATAGCCTCCCGGCAGCCCCTGGGAAGGGAAGAAAGGGGTT | |
| | GGGAACAACCTTCCCCAAGTCCCTTGGGAGCTCCAGG | |
| | CTATCGCGGGCTGCCGG | 15 |
| | CCGGCAGCCCGCGATAG | 16 |
| Adenosine Deaminase | GCTCCCAAGGGACTTGGGGAAGGTTGTTCCCAACCCCTTTCT | 17 |
| Deficiency | TCCCTTCCCAGGGGCTGCCGGGAGGCTATCAAAAGGATCGC | |
| ARG76TRP | CTATGAGTTTGTAGAGATGAAGGCCAAAGAGGGCGTGG | |
| CGG to TGG | CCACGCCCTCTTTGGCCTTCATCTCTACAAACTCATAGGCGAT | 18 |
| | CCTTTTGATAGCCTCCCGGCAGCCCCTGGGAAGGGAAGAAA | |
| | GGGGTTGGGAACAACCTTCCCCAAGTCCCTTGGGAGC | |
| | GGGGCTGCCGGGAGGCT | 19 |
| | AGCCTCCCGGCAGCCCC | 20 |
| Adenosine Deaminase | TTGGGGAAGGTTGTTCCCAACCCCTTTCTTCCCTTCCCAGGG | 21 |
| Deficiency | GCTGCCGGGAGGCTATCAAAAGGATCGCCTATGAGTTTGTAG | |
| LYS80ARG | AGATGAAGGCCAAAGAGGGCGTGGTGTATGTGGAGGT | |
| AAA to AGA | ACCTCCACATACACCACGCCCTCTTTGGCCTTCATCTCTACAA | 22 |
| | ACTCATAGGCGATCCTTTTGATAGCCTCCCGGCAGCCCCTGG | |
| | GAAGGGAAGAAAGGGGTTGGGAACAACCTTCCCCAA | |
| | GGCTATCAAAAGGATCG | 23 |
| | CGATCCTTTTGATAGCC | 24 |
| Adenosine deaminase | GTTGTTCCCAACCCCTTTCTTCCCTTCCCAGGGGCTGCCGGG | 25 |
| deficiency | AGGCTATCAAAAGGATCGCCTATGAGTTTGTAGAGATGAAGG | |
| ALA83ASP | CCAAAGAGGGCGTGGTGTATGTGGAGGTGCGGTACAG | |
| GCC to GAC | CTGTACCGCACCTCCACATACACCACGCCCTCTTTGGCCTTC | 26 |
| | ATCTCTACAAACTCATAGGCGATCCTTTTGATAGCCTCCCGGC | |
| | AGCCCCTGGGAAGGGAAGAAAGGGGTTGGGAACAAC | |
| | AAGGATCGCCTATGAGT | 27 |
| | ACTCATAGGCGATCCTT | 28 |
| Adenosine deaminase | AGGCTATCAAAAGGATCGCCTATGAGTTTGTAGAGATGAAGG | 29 |
| deficiency | CCAAAGAGGGCGTGGTGTATGTGGAGGTGCGGTACAGTCCG | |
| TYR97CYS | CACCTGCTGGCCAACTCCAAAGTGGAGCCAATCCCCTG | |
| TAT to TGT | CAGGGGATTGGCTCCACTTTGGAGTTGGCCAGCAGGTGCGG | 30 |
| | ACTGTACCGCACCTCCACATACACCACGCCCTCTTTGGCCTT | |
| | CATCTCTACAAACTCATAGGCGATCCTTTTGATAGCCT | |
| | CGTGGTGTATGTGGAGG | 31 |
| | CCTCCACATACACCACG | 32 |
| Adenosine deaminase | GGATCGCCTATGAGTTTGTAGAGATGAAGGCCAAAGAGGGCG | 33 |
| deficiency | TGGTGTATGTGGAGGTGCGGTACAGTCCGCACCTGCTGGCC | |
| ARG101GLN | AACTCCAAAGTGGAGCCAATCCCCTGGAACCAGGCTGA | |
| CGG to CAG | TCAGCCTGGTTCCAGGGGATTGGCTCCACTTTGGAGTTGGCC | 34 |
| | AGCAGGTGCGGACTGTACCGCACCTCCACATACACCACGCC | |
| | CTCTTTGGCCTTCATCTCTACAAACTCATAGGCGATCC | |
| | GGAGGTGCGGTACAGTC | 35 |
| | GACTGTACCGCACCTCC | 36 |
| Adenosine deaminase | GGATCGCCTATGAGTTTGTAGAGATGAAGGCCAAAGAGGGCG | 37 |
| deficiency | TGGTGTATGTGGAGGTGCGGTACAGTCCGCACCTGCTGGCC | |
| ARG101LEU | AACTCCAAAGTGGAGCCAATCCCCTGGAACCAGGCTGA | |
| CGG to CTG | TCAGCCTGGTTCCAGGGGATTGGCTCCACTTTGGAGTTGGCC | 38 |
| | AGCAGGTGCGGACTGTACCGCACCTCCACATACACCACGCC | |
| | CTCTTTGGCCTTCATCTCTACAAACTCATAGGCGATCC | |
| | GGAGGTGCGGTACAGTC | 39 |
| | GACTGTACCGCACCTCC | 40 |
| Adenosine deaminase | AGGATCGCCTATGAGTTTGTAGAGATGAAGGCCAAAGAGGGC | 41 |
| deficiency | GTGGTGTATGTGGAGGTGCGGTACAGTCCGCACCTGCTGGC | |
| ARG101TRP | CAACTCCAAAGTGGAGCCAATCCCCTGGAACCAGGCTG | |
| CGG to TGG | CAGCCTGGTTCCAGGGGATTGGCTCCACTTTGGAGTTGGCCA | 42 |
| | GCAGGTGCGGACTGTACCGCACCTCCACATACACCACGCCCT | |
| | CTTTGGCCTTCATCTCTACAAACTCATAGGCGATCCT | |
| | TGGAGGTGCGGTACAGT | 43 |
| | ACTGTACCGCACCTCCA | 44 |
| Adenosine deaminase | ATGAGTTTGTAGAGATGAAGGCCAAAGAGGGCGTGGTGTATG | 45 |
| deficiency | TGGAGGTGCGGTACAGTCCGCACCTGCTGGCCAACTCCAAA | |
| PRO104LEU | GTGGAGCCAATCCCCTGGAACCAGGCTGAGTGAGTGAT | |
| CCG to CTG | ATCACTCACTCAGCCTGGTTCCAGGGGATTGGCTCCACTTTG | 46 |
| | GAGTTGGCCAGCAGGTGCGGACTGTACCGCACCTCCACATA | |
| | CACCACGCCCTCTTTGGCCTTCATCTCTACAAACTCAT | |
| | GTACAGTCCGCACCTGC | 47 |
| | GCAGGTGCGGACTGTAC | 48 |
| Adenosine deaminase | TTTGTAGAGATGAAGGCCAAAGAGGGCGTGGTGTATGTGGAG | 49 |
| deficiency | GTGCGGTACAGTCCGCACCTGCTGGCCAACTCCAAAGTGGA | |
| LEU106VAL | GCCAATCCCCTGGAACCAGGCTGAGTGAGTGATGGGCC | |
| CTG to GTG | GGCCCATCACTCACTCAGCCTGGTTCCAGGGGATTGGCTCCA | 50 |
| | CTTTGGAGTTGGCCAGCAGGTGCGGACTGTACCGCACCTCCA | |
| | CATACACCACGCCCTCTTTGGCCTTCATCTCTACAAA | |
| | GTCCGCACCTGCTGGCC | 51 |
| | GGCCAGCAGGTGCGGAC | 52 |
| Adenosine deaminase | TAGAGATGAAGGCCAAAGAGGGCGTGGTGTATGTGGAGGTG | 53 |
| deficiency | CGGTACAGTCCGCACCTGCTGGCCAACTCCAAAGTGGAGCC | |
| LEU107PRO | AATCCCCTGGAACCAGGCTGAGTGAGTGATGGGCCTGGA | |
| CTG to CCG | TCCAGGCCCATCACTCACTCAGCCTGGTTCCAGGGGATTGGC | 54 |
| | TCCACTTTGGAGTTGGCCAGCAGGTGCGGACTGTACCGCAC | |
| | CTCCACATACACCACGCCCTCTTTGGCCTTCATCTCTA | |
| | GCACCTGCTGGCCAACT | 55 |
| | AGTTGGCCAGCAGGTGC | 56 |
| Adenosine deaminase | GCCTTCCTTTTGCCTCAGGCCCATCCCTACTCCTCTCCTCACA | 57 |
| deficiency | CAGAGGGGACCTCACCCCAGACGAGGTGGTGGCCCTAGTGG | |
| PRO126GLN | GCCAGGGCCTGCAGGAGGGGGAGCGAGACTTCGGGGT | |
| CCA to CAA | ACCCCGAAGTCTCGCTCCCCCTCCTGCAGGCCCTGGCCCAC | 58 |
| | TAGGGCCACCACCTCGTCTGGGGTGAGGTCCCCTCTGTGTG | |
| | AGGAGAGGAGTAGGGATGGGCCTGAGGCAAAAGGAAGGC | |
| | CCTCACCCCAGACGAGG | 59 |
| | CCTCGTCTGGGGTGAGG | 60 |
| Adenosine deaminase | TTTGCCTCAGGCCCATCCCTACTCCTCTCCTCACACAGAGGG | 61 |
| deficiency | GACCTCACCCCAGACGAGGTGGTGGCCCTAGTGGGCCAGGG | |
| VAL129MET | CCTGCAGGAGGGGGAGCGAGACTTCGGGGTCAAGGCCC | |
| GTG to ATG | GGGCCTTGACCCCGAAGTCTCGCTCCCCCTCCTGCAGGCCC | 62 |
| | TGGCCCACTAGGGCCACCACCTCGTCTGGGGTGAGGTCCCC | |
| | TCTGTGTGAGGAGAGGAGTAGGGATGGGCCTGAGGCAAA | |
| | CAGACGAGGTGGTGGCC | 63 |
| | GGCCACCACCTCGTCTG | 64 |
| Adenosine deaminase | ACAGAGGGGACCTCACCCCAGACGAGGTGGTGGCCCTAGTG | 65 |
| deficiency | GGCCAGGGCCTGCAGGAGGGGGAGCGAGACTTCGGGGTCA | |
| GLY140GLU | AGGCCCGGTCCATCCTGTGCTGCATGCGCCACCAGCCCAG | |
| GGG to GAG | CTGGGCTGGTGGCGCATGCAGCACAGGATGGACCGGGCCTT | 66 |
| | GACCCCGAAGTCTCGCTCCCCCTCCTGCAGGCCCTGGCCCA | |
| | CTAGGGCCACCACCTCGTCTGGGGTGAGGTCCCCTCTGT | |
| | GCAGGAGGGGGAGCGAG | 67 |
| | CTCGCTCCCCCTCCTGC | 68 |
| Adenosine deaminase | GGGACCTCACCCCAGACGAGGTGGTGGCCCTAGTGGGCCAG | 69 |
| deficiency | GGCCTGCAGGAGGGGGAGCGAGACTTCGGGGTCAAGGCCC | |
| ARG142GLN | GGTCCATCCTGTGCTGCATGCGCCACCAGCCCAGTGAGTA | |
| CGA to CAA | TACTCACTGGGCTGGTGGCGCATGCAGCACAGGATGGACCG | 70 |
| | GGCCTTGACCCCGAAGTCTCGCTCCCCCTCCTGCAGGCCCT | |
| | GGCCCACTAGGGCCACCACCTCGTCTGGGGTGAGGTCCC | |
| | GGGGGAGCGAGACTTCG | 71 |
| | CGAAGTCTCGCTCCCCC | 72 |
| Adenosine deaminase | GGGGACCTCACCCCAGACGAGGTGGTGGCCCTAGTGGGCCA | 73 |
| deficiency | GGGCCTGCAGGAGGGGGAGCGAGACTTCGGGGTCAAGGCC | |
| ARG142TERM | CGGTCCATCCTGTGCTGCATGCGCCACCAGCCCAGTGAGT | |
| CGA to TGA | ACTCACTGGGCTGGTGGCGCATGCAGCACAGGATGGACCGG | 74 |
| | GCCTTGACCCCGAAGTCTCGCTCCCCCTCCTGCAGGCCCTG | |
| | GCCCACTAGGGCCACCACCTCGTCTGGGGTGAGGTCCCC | |
| | AGGGGGAGCGAGACTTC | 75 |
| | GAAGTCTCGCTCCCCCT | 76 |
| Adenosine deaminase | TGGTGGCCCTAGTGGGCCAGGGCCTGCAGGAGGGGGAGCG | 77 |
| deficiency | AGACTTCGGGGTCAAGGCCCGGTCCATCCTGTGCTGCATGC | |
| ARG149GLN | GCCACCAGCCCAGTGAGTAGGATCACCGCCCTGCCCAGGG | |
| CGG to CAG | CCCTGGGCAGGGCGGTGATCCTACTCACTGGGCTGGTGGCG | 78 |
| | CATGCAGCACAGGATGGACCGGGCCTTGACCCCGAAGTCTC | |
| | GCTCCCCCTCCTGCAGGCCCTGGCCCACTAGGGCCACCA | |
| | CAAGGCCCGGTCCATCC | 79 |
| | GGATGGACCGGGCCTTG | 80 |
| Adenosine deaminase | GTGGTGGCCCTAGTGGGCCAGGGCCTGCAGGAGGGGGAGC | 81 |
| deficiency | GAGACTTCGGGGTCAAGGCCCGGTCCATCCTGTGCTGCATG | |
| ARG149TRP | CGCCACCAGCCCAGTGAGTAGGATCACCGCCCTGCCCAGG | |
| CGG to TGG | CCTGGGCAGGGCGGTGATCCTACTCACTGGGCTGGTGGCGC | 82 |
| | ATGCAGCACAGGATGGACCGGGCCTTGACCCCGAAGTCTCG | |
| | CTCCCCCTCCTGCAGGCCCTGGCCCACTAGGGCCACCAC | |
| | TCAAGGCCCGGTCCATC | 83 |
| | GATGGACCGGGCCTTGA | 84 |
| Adenosine deaminase | CTAGTGGGCCAGGGCCTGCAGGAGGGGGAGCGAGACTTCG | 85 |
| deficiency | GGGTCAAGGCCCGGTCCATCCTGTGCTGCATGCGCCACCAG | |
| LEU152MET | CCCAGTGAGTAGGATCACCGCCCTGCCCAGGGCCGCCCGT | |
| CTG to ATG | ACGGGCGGCCCTGGGCAGGGCGGTGATCCTACTCACTGGGC | 86 |
| | TGGTGGCGCATGCAGCACAGGATGGACCGGGCCTTGACCCC | |
| | GAAGTCTCGCTCCCCCTCCTGCAGGCCCTGGCCCACTAG | |
| | GGTCCATCCTGTGCTGC | 87 |
| | GCAGCACAGGATGGACC | 88 |
| Adenosine deaminase | GGCCTGCAGGAGGGGGAGCGAGACTTCGGGGTCAAGGCCC | 89 |
| deficiency | GGTCCATCCTGTGCTGCATGCGCCACCAGCCCAGTGAGTAG | |
| ARG156CYS | GATCACCGCCCTGCCCAGGGCCGCCCGTCTCACCCTGGCC | |
| CGC to TGC | GGCCAGGGTGAGACGGGCGGCCCTGGGCAGGGCGGTGATC | 90 |
| | CTACTCACTGGGCTGGTGGCGCATGCAGCACAGGATGGACC | |
| | GGGCCTTGACCCCGAAGTCTCGCTCCCCCTCCTGCAGGCC | |
| | GCTGCATGCGCCACCAG | 91 |
| | CTGGTGGCGCATGCAGC | 92 |
| Adenosine deaminase | GCCTGCAGGAGGGGGAGCGAGACTTCGGGGTCAAGGCCCG | 93 |
| deficiency | GTCCATCCTGTGCTGCATGCGCCACCAGCCCAGTGAGTAGGA | |
| ARG156HIS | TCACCGCCCTGCCCAGGGCCGCCCGTCTCACCCTGGCCC | |
| CGC to CAC | GGGCCAGGGTGAGACGGGCGGCCCTGGGCAGGGCGGTGAT | 94 |
| | CCTACTCACTGGGCTGGTGGCGCATGCAGCACAGGATGGAC | |
| | CGGGCCTTGACCCCGAAGTCTCGCTCCCCCTCCTGCAGGC | |
| | CTGCATGCGCCACCAGC | 95 |
| | GCTGGTGGCGCATGCAG | 96 |
| Adenosine deaminase | CTGCCCACAGACTGGTCCCCCAAGGTGGTGGAGCTGTGTAA | 97 |
| deficiency | GAAGTACCAGCAGCAGACCGTGGTAGCCATTGACCTGGCTG | |
| VAL177MET | GAGATGAGACCATCCCAGGAAGCAGCCTCTTGCCTGGAC | |
| GTG to ATG | GTCCAGGCAAGAGGCTGCTTCCTGGGATGGTCTCATCTCCAG | 98 |
| | CCAGGTCAATGGCTACCACGGTCTGCTGCTGGTACTTCTTAC | |
| | ACAGCTCCACCACCTTGGGGGACCAGTCTGTGGGCAG | |
| | AGCAGACCGTGGTAGCC | 99 |
| | GGCTACCACGGTCTGCT | 100 |
| Adenosine deaminase | CAGACTGGTCCCCCAAGGTGGTGGAGCTGTGTAAGAAGTACC | 101 |
| deficiency | AGCAGCAGACCGTGGTAGCCATTGACCTGGCTGGAGATGAG | |
| ALA179ASP | ACCATCCCAGGAAGCAGCCTCTTGCCTGGACATGTCCA | |
| GCC to GAC | TGGACATGTCCAGGCAAGAGGCTGCTTCCTGGGATGGTCTCA | 102 |
| | TCTCCAGCCAGGTCAATGGCTACCACGGTCTGCTGCTGGTAC | |
| | TTCTTACACAGCTCCACCACCTTGGGGGACCAGTCTG | |
| | CGTGGTAGCCATTGACC | 103 |
| | GGTCAATGGCTACCACG | 104 |
| Adenosine deaminase | CCATTGACCTGGCTGGAGATGAGACCATCCCAGGAAGCAGC | 105 |
| deficiency | CTCTTGCCTGGACATGTCCAGGCCTACCAGGTGGGTCCTGTG | |
| GLN199PRO | AGAAGGAATGGAGAGGCTGGCCCTGGGTGAGCTTGTCT | |
| CAG to CCG | AGACAAGCTCACCCAGGGCCAGCCTCTCCATTCCTTCTCACA | 106 |
| | GGACCCACCTGGTAGGCCTGGACATGTCCAGGCAAGAGGCT | |
| | GCTTCCTGGGATGGTCTCATCTCCAGCCAGGTCAATGG | |
| | ACATGTCCAGGCCTACC | 107 |
| | GGTAGGCCTGGACATGT | 108 |
| Adenosine deaminase | GCTAGGGCACCCATGACCTGGCTCTCCCCCTTCCAGGAGGC | 109 |
| deficiency | TGTGAAGAGCGGCATTCACCGTACTGTCCACGCCGGGGAGG | |
| ARG211CYS | TGGGCTCGGCCGAAGTAGTAAAAGAGGTGAGGGCCTGGG | |
| CGT to TGT | CCCAGGCCCTCACCTCTTTTACTACTTCGGCCGAGCCCACCT | 110 |
| | CCCCGGCGTGGACAGTACGGTGAATGCCGCTCTTCACAGCC | |
| | TCCTGGAAGGGGGAGAGCCAGGTCATGGGTGCCCTAGC | |
| | GCATTCACCGTACTGTC | 111 |
| | GACAGTACGGTGAATGC | 112 |
| Adenosine deaminase | CTAGGGCACCCATGACCTGGCTCTCCCCCTTCCAGGAGGCT | 113 |
| deficiency | GTGAAGAGCGGCATTCACCGTACTGTCCACGCCGGGGAGGT | |
| ARG211HIS | GGGCTCGGCCGAAGTAGTAAAAGAGGTGAGGGCCTGGGC | |
| CGT to CAT | GCCCAGGCCCTCACCTCTTTTACTACTTCGGCCGAGCCCACC | 114 |
| | TCCCCGGCGTGGACAGTACGGTGAATGCCGCTCTTCACAGC | |
| | CTCCTGGAAGGGGGAGAGCCAGGTCATGGGTGCCCTAG | |
| | CATTCACCGTACTGTCC | 115 |
| | GGACAGTACGGTGAATG | 116 |
| Adenosine deaminase | ATGACCTGGCTCTCCCCCTTCCAGGAGGCTGTGAAGAGCGG | 117 |
| deficiency | CATTCACCGTACTGTCCACGCCGGGGAGGTGGGCTCGGCCG | |
| ALA215THR | AAGTAGTAAAAGAGGTGAGGGCCTGGGCTGGCCATGGGG | |
| GCC to ACC | CCCCATGGCCAGCCCAGGCCCTCACCTCTTTTACTACTTCGG | 118 |
| | CCGAGCCCACCTCCCCGGCGTGGACAGTACGGTGAATGCCG | |
| | CTCTTCACAGCCTCCTGGAAGGGGGAGAGCCAGGTCAT | |
| | CTGTCCACGCCGGGGAG | 119 |
| | CTCCCCGGCGTGGACAG | 120 |
| Adenosine deaminase | ACCTGGCTCTCCCCCTTCCAGGAGGCTGTGAAGAGCGGCATT | 121 |
| deficiency | CACCGTACTGTCCACGCCGGGGAGGTGGGCTCGGCCGAAGT | |
| GLY216ARG | AGTAAAAGAGGTGAGGGCCTGGGCTGGCCATGGGGTCC | |
| GGG to AGG | GGACCCCATGGCCAGCCCAGGCCCTCACCTCTTTTACTACTT | 122 |
| | CGGCCGAGCCCACCTCCCCGGCGTGGACAGTACGGTGAATG | |
| | CCGCTCTTCACAGCCTCCTGGAAGGGGGAGAGCCAGGT | |
| | TCCACGCCGGGGAGGTG | 123 |
| | CACCTCCCCGGCGTGGA | 124 |
| Adenosine deaminase | TGGCTCTCCCCCTTCCAGGAGGCTGTGAAGAGCGGCATTCAC | 125 |
| deficiency | CGTACTGTCCACGCCGGGGAGGTGGGCTCGGCCGAAGTAGT | |
| GLU217LYS | AAAAGAGGTGAGGGCCTGGGCTGGCCATGGGGTCCCTC | |
| GAG to AAG | GAGGGACCCCATGGCCAGCCCAGGCCCTCACCTCTTTTACTA | 126 |
| | CTTCGGCCGAGCCCACCTCCCCGGCGTGGACAGTACGGTGA | |
| | ATGCCGCTCTTCACAGCCTCCTGGAAGGGGGAGAGCCA | |
| | ACGCCGGGGAGGTGGGC | 127 |
| | GCCCACCTCCCCGGCGT | 128 |
| Adenosine deaminase | CTGCCTCCTCCCATACTTGGCTCTATTCTGCTTCTCTACAGGC | 129 |
| deficiency | TGTGGACATACTCAAGACAGAGCGGCTGGGACACGGCTACC | |
| THR233ILE | ACACCCTGGAAGACCAGGCCCTTTATAACAGGCTGCG | |
| ACA to ATA | CGCAGCCTGTTATAAAGGGCCTGGTCTTCCAGGGTGTGGTAG | 130 |
| | CCGTGTCCCAGCCGCTCTGTCTTGAGTATGTCCACAGCCTGT | |
| | AGAGAAGCAGAATAGAGCCAAGTATGGGAGGAGGCAG | |
| | ACTCAAGACAGAGCGGC | 131 |
| | GCCGCTCTGTCTTGAGT | 132 |
| Adenosine deaminase | CAGAGCGGCTGGGACACGGCTACCACACCCTGGAAGACCAG | 133 |
| deficiency | GCCCTTTATAACAGGCTGCGGCAGGAAAACATGCACTTCGAG | |
| ARG253PRO | GTAAGCGGGCCAGGGAGTGGGGAGGAACCATCCCCGGC | |
| CGG to CCG | GCCGGGGATGGTTCCTCCCCACTCCCTGGCCCGCTTACCTC | 134 |
| | GAAGTGCATGTTTTCCTGCCGCAGCCTGTTATAAAGGGCCTG | |
| | GTCTTCCAGGGTGTGGTAGCCGTGTCCCAGCCGCTCTG | |
| | CAGGCTGCGGCAGGAAA | 135 |
| | TTTCCTGCCGCAGCCTG | 136 |
| Adenosine deaminase | GAGCGGCTGGGACACGGCTACCACACCCTGGAAGACCAGGC | 137 |
| deficiency | CCTTTATAACAGGCTGCGGCAGGAAAACATGCACTTCGAGGT | |
| GLN254TERM | AAGCGGGCCAGGGAGTGGGGAGGAACCATCCCCGGCTG | |
| CAG to TAG | CAGCCGGGGATGGTTCCTCCCCACTCCCTGGCCCGCTTACC | 138 |
| | CGAAGTGCATGTTTTCCTGCCGCAGCCTGTTATAAAGGGCCT | |
| | GGTCTTCCAGGGTGTGGTAGCCGTGTCCCAGCCGCTC | |
| | GGCTGCGGCAGGAAAAC | 139 |
| | GTTTTCCTGCCGCAGCC | 140 |
| Adenosine deaminase | CCACACACCTGCTCTTCCAGATCTGCCCCTGGTCCAGCTACC | 141 |
| deficiency | TCACTGGTGCCTGGAAGCCGGACACGGAGCATGCAGTCATTC | |
| PRO274LEU | GGTGAGCTCTGTTCCCCTGGGCCTGTTCAATTTTGTT | |
| CCG to CTG | AACAAAATTGAACAGGCCCAGGGGAACAGAGCTCACCGAATG | 142 |
| | ACTGCATGCTCCGTGTCCGGCTTCCAGGCACCAGTGAGGTAG | |
| | CTGGACCAGGGGCAGATCTGGAAGAGCAGGTGTGTGG | |
| | CTGGAAGCCGGACACGG | 143 |
| | CCGTGTCCGGCTTCCAG | 144 |
| Adenosine deaminase | GGAGGCTGATTCTCTCCTCCTCCCTCTTCTGCAGGCTCAAAA | 145 |
| deficiency | ATGACCAGGCTAACTACTCGCTCAACACAGATGACCCGCTCA | |
| SER291LEU | TCTTCAAGTCCACCCTGGACACTGATTACCAGATGAC | |
| TCG to TTG | GTCATCTGGTAATCAGTGTCCAGGGTGGACTTGAAGATGAGC | 146 |
| | GGGTCATCTGTGTTGAGCGAGTAGTTAGCCTGGTCATTTTTGA | |
| | GCCTGCAGAAGAGGGAGGAGGAGAGAATCAGCCTCC | |
| | TAACTACTCGCTCAACA | 147 |
| | TGTTGAGCGAGTAGTTA | 148 |
| Adenosine deaminase | CCTCCCTCTTCTGCAGGCTCAAAAATGACCAGGCTAACTACTC | 149 |
| deficiency | GCTCAACACAGATGACCCGCTCATCTTCAAGTCCACCCTGGA | |
| PRO297GLN | CACTGATTACCAGATGACCAAACGGGACATGGGCTT | |
| CCG to CAG | AAGCCCATGTCCCGTTTGGTCATCTGGTAATCAGTGTCCAGG | 150 |
| | GTGGACTTGAAGATGAGCGGGTCATCTGTGTTGAGCGAGTAG | |
| | TTAGCCTGGTCATTTTTGAGCCTGCAGAAGAGGGAGG | |
| | AGATGACCCGCTCATCT | 151 |
| | AGATGAGCGGGTCATCT | 152 |
| Adenosine deaminase | AAAATGACCAGGCTAACTACTCGCTCAACACAGATGACCCGC | 153 |
| deficiency | TCATCTTCAAGTCCACCCTGGACACTGATTACCAGATGACCAA | |
| LEU304ARG | ACGGGACATGGGCTTTACTGAAGAGGAGTTTAAAAG | |
| CTG to CGG | CTTTTAAACTCCTCTTCAGTAAAGCCCATGTCCCGTTTGGTCA | 154 |
| | TCTGGTAATCAGTGTCCAGGGTGGACTTGAAGATGAGCGGGT | |
| | CATCTGTGTTGAGCGAGTAGTTAGCCTGGTCATTTT | |
| | GTCCACCCTGGACACTG | 155 |
| | CAGTGTCCAGGGTGGAC | 156 |
| Adenosine deaminase | GCCTTCTTTGTTCTCTGGTTCCATGTTGTCTGCCATTCTGGCC | 157 |
| deficiency | TTTCCAGAACATCAATGCGGCCAAATCTAGTTTCCTCCCAGAA | |
| ALA329VAL | GATGAAAAGAGGGAGCTTCTCGACCTGCTCTATAA | |
| C-to-T at base 1081 | TTATAGAGCAGGTCGAGAAGCTCCCTCTTTTCATCTTCTGGGA | 158 |
| | GGAAACTAGATTTGGCCGCATTGATGTTCTGGAAAGGCCAGA | |
| | ATGGCAGACAACATGGAACCAGAGAACAAAGAAGGC | |
| | CATCAATGCGGCCAAAT | 159 |
| | ATTTGGCCGCATTGATG | 160 |

### EXAMPLE 6

### P53 Mutations

The p53 gene codes for a protein that acts as a transcription factor and serves as a key regulator of the cell cycle. Mutation in this gene is probably the most significant genetic change characterizing the transformation of cells from normalcy to malignancy.

Inactivation of p53 by mutation disrupts the cell cycle which, in turn, sets the stage for tumor formation. Mutations in the p53 gene are among the most commonly diagnosed genetic disorders, occurring in as many as 50% of cancer patients. For some types of cancer, most notably of the breast, lung and colon, p53 mutations are the predominant genetic alternations found thus far. These mutations are associated with genomic instability and thus an increased susceptibility to cancer. Some p53 lesions result in malignancies that are resistant to the most widely used therapeutic regimens and therefore demand more aggressive treatment.

That p53 is associated with different malignant tumors is illustrated in the Li-Fraumeni autosomal dominant hereditary disorder characterized by familial multiple tumors due to mutation in the p53 gene. Affected individuals can develop one or more tumors, including: brain (12%); soft-tissue sarcoma (12%); breast cancer (25%); adrenal tumors (1%); bone cancer (osteosarcoma) (6%); cancer of the lung, prostate, pancreas, and colon as well as lymphoma and melanoma can also occur.

Certain of the most frequently mutated codons are codons 175, 248 and 273, however a variety of oligonucleotides are described below in the attached table.

TABLE 7
p53 Mutations and Genome-Correcting Oligos

| | | |
|---|---|---|
| Clinical Phenotype & | | SEQ ID |
| Mutation | Correcting Oligos | NO: |

| | | |
|---|---|---|
| In 2 families with | GACTGTACCACCATCCACTACAACTACATGTGTAACAGTTCCT | 161 |
| Li-Fraumeni | GCATGGGCGGCATGAACCGGAGGCCCATCCTCACCATCATC | |
| syndrome, there was a | ACACTGGAAGACTCCAGGTCAGGAGCCACTTGCCACC | |
| C-to-T mutation at the | GGTGGCAAGTGGCTCCTGACCTGGAGTCTTCCAGTGTGATGA | 162 |
| first nucleotide of | TGGTGAGGATGGGCCTCCGGTTCATGCCGCCCATGCAGGAA | |
| codon 248 which | CTGTTACACATGTAGTTGTAGTGGATGGTGGTACAGTC | |
| changed arginine to | GCATGAACCGGAGGCCC | 163 |
| tryptophan. | GGGCCTCCGGTTCATGC | 164 |
| In a family with the | TGTAACAGTTCCTGCATGGGCGGCATGAACCGGAGGCCCAT | 165 |
| Li-Fraumeni | CCTCACCATCATCACACTGGAAGACTCCAGGTCAGGAGCCAC | |
| syndrome, a G-to-A | TTGCCACCCTGCACACTGGCCTGCTGTGCCCCAGCCTC | |
| mutation at the first | GAGGCTGGGGCACAGCAGGCCAGTGTGCAGGGTGGCAAGT | 166 |
| nucleotide of codon | GGCTCCTGACCTGGAGTCTTCCAGTGTGATGATGGTGAGGAT | |
| 258 resulting in the | GGGCCTCCGGTTCATGCCGCCCATGCAGGAACTGTTACA | |
| substitution of lysine | TCACACTGGAAGACTCC | 167 |
| for glutamic acid. | GGAGTCTTCCAGTGTGA | 168 |
| In a family with the | GTTGGCTCTGACTGTACCACCATCCACTACAACTACATGTGTA | 169 |
| Li-Fraumeni | ACAGTTCCTGCATGGGCGGCATGAACCGGAGGCCCATCCTC | |
| syndrome, a G-to-T | ACCATCATCACACTGGAAGACTCCAGGTCAGGAGCCA | |
| mutation at | | |
| the first nucleotide of | | |
| codon 245 resulting in | | |
| the substitution of | | |
| cysteine for glycine. | | |
| A gly245-to-ser, | TGGCTCCTGACCTGGAGTCTTCCAGTGTGATGATGGTGAGGA | 170 |
| GGC-to-AGC, | TGGGCCTCCGGTTCATGCCGCCCATGCAGGAACTGTTACACA | |
| mutation was found in | TGTAGTTGTAGTGGATGGTGGTACAGTCAGAGCCAAC | |
| a patient in whom | GCATGGGCGGCATGAAC | 171 |
| osteosarcoma was | GTTCATGCCGCCCATGC | 172 |
| diagnosed at the age | | |
| of 18 years. | | |
| In a family with the | TCCACTACAACTACATGTGTAACAGTTCCTGCATGGGCGGCA | 173 |
| Li-Fraumeni | TGAACCGGAGGCCCATCCTCACCATCATCACACTGGAAGACT | |
| syndrome, a germline | CCAGGTCAGGAGCCACTTGCCACCCTGCACACTGGCC | |
| mutation at codon 252: | GGCCAGTGTGCAGGGTGGCAAGTGGCTCCTGACCTGGAGTC | 174 |
| a T-to-C change at the | TTCCAGTGTGATGATGGTGAGGATGGGCCTCCGGTTCATGCC | |
| second position | GCCCATGCAGGAACTGTTACACATGTAGTTGTAGTGGA | |
| resulted in substitution | GCCCATCCTCACCATCA | 175 |
| of proline for leucine. | TGATGGTGAGGATGGGC | 176 |
| Researchers analyzed | TACCACCATCCACTACAACTACATGTGTAACAGTTCCTGCATG | 177 |
| for mutations in p53 | GGCGGCATGAACCGGAGGCCCATCCTCACCATCATCACACT | |
| hepatocellular | GGAAGACTCCAGGTCAGGAGCCACTTGCCACCCTGCA | |
| hepatocellular | TGCAGGGTGGCAAGTGGCTCCTGACCTGGAGTCTTCCAGTGT | 178 |
| carcinomas from | GATGATGGTGAGGATGGGCCTCCGGTTCATGCCGCCCATGC | |
| patients in Qidong, an | AGGAACTGTTACACATGTAGTTGTAGTGGATGGTGGTA | |
| area of high incidence | AACCGGAGGCCCATCCT | 179 |
| in China, in which both | AGGATGGGCCTCCGGTT | 180 |
| hepatitis B virus and | | |
| aflatoxin B1 are risk | | |
| factors. Eight of 16 | | |
| tumors had a point | | |
| mutation at the third | | |
| base position of codon | | |
| 249. The G-to-T | | |
| mutation at codon 249 | | |
| led to a change from | | |
| arginine to serine | | |
| (AGG to AGT). | | |
| In cases of | CTGGCCAAGACCTGCCCTGTGCAGCTGTGGGTTGATTCCACA | 181 |
| hepatocellular | CCCCCGCCCGGCACCCGCGTCCGCGCCATGGCCATCTACAA | |
| carcinoma in southern | GCAGTCACAGCACATGACGGAGGTTGTGAGGCGCTGCC | |
| Africa, a G-to-T | GGCAGCGCCTCACAACCTCCGTCATGTGCTGTGACTGCTTGT | 182 |
| substitution in codon | AGATGGCCATGGCGCGGACGCGGGTGCCGGGCGGGGGTGT | |
| 157 resulting in a | GGAATCAACCCACAGCTGCACAGGGCAGGTCTTGGCCAG | |
| change from valine to | GCACCCGCGTCCGCGCC | 183 |
| phenylalanine. | GGCGCGGACGCGGGTGC | 184 |
| In a family with | TTGGCTCTGACTGTACCACCATCCACTACAACTACATGTGTAA | 185 |
| Li-Fraumeni in which | CAGTTCCTGCATGGGCGGCATGAACCGGAGGCCCATCCTCA | |
| noncancerous skin | CCATCATCACACTGGAAGACTCCAGGTCAGGAGCCAC | |
| fibroblasts from | GTGGCTCCTGACCTGGAGTCTTCCAGTGTGATGATGGTGAGG | 186 |
| affected individuals | ATGGGCCTCCGGTTCATGCCGCCCATGCAGGAACTGTTACAC | |
| showed an unusual | ATGTAGTTGTAGTGGATGGTGGTACAGTCAGAGCCAA | |
| radiation-resistant | CATGGGCGGCATGAACC | 187 |
| phenotype, a point | GGTTCATGCCGCCCATG | 188 |
| mutation in codon 245 | | |
| of the P53 gene. A | | |
| change from GGC to | | |
| GAC predicted | | |
| substitution of aspartic | | |
| acid for glycine. | | |
| In 2 of 8 families with | ACTGTACCACCATCCACTACAACTACATGTGTAACAGTTCCTG | 189 |
| Li-Fraumeni | CATGGGCGGCATGAACCGGAGGCCCATCCTCACCATCATCA | |
| syndrome, a mutation | CACTGGAAGACTCCAGGTCAGGAGCCACTTGCCACCC | |
| syndrome, a mutation | GGGTGGCAAGTGGCTCCTGACCTGGAGTCTTCCAGTGTGAT | 190 |
| in codon 248: a | GATGGTGAGGATGGGCCTCCGGTTCATGCCGCCCATGCAGG | |
| CGG-to-CAG change | AACTGTTACACATGTAGTTGTAGTGGATGGTGGTACAGT | |
| resulting in substitution | CATGAACCGGAGGCCCA | 191 |
| of glutamine for | TGGGCCTCCGGTTCATG | 192 |
| arginine. | | |
| In 9 members of an | CCCTGACTTTCAACTCTGTCTCCTTCCTCTTCCTACAGTACTC | 193 |
| extended family with | CCCTGCCCTCAACAAGATGTTTTGCCAACTGGCCAAGACCTG | |
| Li-Fraumeni | CCCTGTGCAGCTGTGGGTTGATTCCACACCCCCGCC | |
| syndrome, a germline | GGCGGGGGTGTGGAATCAACCCACAGCTGCACAGGGCAGGT | 194 |
| mutation at codon 133 | CTTGGCCAGTTGGCAAAACATCTTGTTGAGGGCAGGGGAGTA | |
| (ATG-to-ACG), | CTGTAGGAAGAGGAAGGAGACAGAGTTGAAAGTCAGGG | |
| resulted in the | CAACAAGATGTTTTGCC | 195 |
| substitution of | GGCAAAACATCTTGTTG | 196 |
| threonine for | | |
| methionine (M133T), | | |
| and completely | | |
| cosegregated with the | | |
| cancer syndrome. | | |
| In 1 pedigree | TCTTGCTTCTCTTTTCCTATCCTGAGTAGTGGTAATCTACTGG | 197 |
| consistent with the | GACGGAACAGCTTTGAGGTGCGTGTTTGTGCCTGTCCTGGGA | |
| Li-Fraumeni | GAGACCGGCGCACAGAGGAAGAGAATCTCCGCAAGA | |
| syndrome, a germline | TCTTGCGGAGATTCTCTTCCTCTGTGCGCCGGTCTCTCCCAG | 198 |
| G-to-T transversion at | GACAGGCACAAACACGCACCTCAAAGCTGTTCCGTCCCAGTA | |
| codon 272 (valine to | GATTACCACTACTCAGGATAGGAAAAGAGAAGCAAGA | |
| leucine) was found. | GCTTTGAGGTGCGTGTT | 199 |
| | AACACGCACCTCAAAGC | 200 |
| A ser241-to-phe | TTATCTCCTAGGTTGGCTCTGACTGTACCACCATCCACTACAA | 201 |
| mutation due to a | CTACATGTGTAACAGTTCCTGCATGGGCGGCATGAACCGGAG | |
| TCC-to-TTC change | GCCCATCCTCACCATCATCACACTGGAAGACTCCAG | |
| was found in a patient | CTGGAGTCTTCCAGTGTGATGATGGTGAGGATGGGCCTCCG | 202 |
| with hepatoblastoma | GTTCATGCCGCCCATGCAGGAACTGTTACACATGTAGTTGTA | |
| and multiple foci of | GTGGATGGTGGTACAGTCAGAGCCAACCTAGGAGATAA | |
| osteosarcoma. | TAACAGTTCCTGCATGG | 203 |
| | CCATGCAGGAACTGTTA | 204 |
| An AAG-to-TAG | CAGAAAACCTACCAGGGCAGCTACGGTTTCCGTCTGGGCTTC | 205 |
| change of codon 120, | TTGCATTCTGGGACAGCCAAGTCTGTGACTTGCACGGTCAGT | |
| resulting in conversion | TGCCCTGAGGGGCTGGCTTCCATGAGACTTCAATGCC | |
| resulting in conversion | GGCATTGAAGTCTCATGGAAGCCAGCCCCTCAGGGCAACTGA | 206 |
| from lysine to a stop | CCGTGCAAGTCACAGACTTGGCTGTCCCAGAATGCAAGAAGC | |
| codon, was found in a | CCAGACGGAAACCGTAGCTGCCCTGGTAGGTTTTCTG | |
| patient with | GGACAGCCAAGTCTGTG | 207 |
| osteosarcoma and | CACAGACTTGGCTGTCC | 208 |
| adenocarcinoma of the | | |
| lung at age 18 and | | |
| brain tumor (glioma) at | | |
| the age of 27. | | |
| A CGG-to-TGG | GGTAATCTACTGGGACGGAACAGCTTTGAGGTGCGTGTTTGT | 209 |
| change at codon 282, | GCCTGTCCTGGGAGAGACCGGCGCACAGAGGAAGAGAATCT | |
| resulting in the | CCGCAAGAAAGGGGAGCCTCACCACGAGCTGCCCCCAG | |
| substitution of | CTGGGGGCAGCTCGTGGTGAGGCTCCCCTTTCTTGCGGAGA | 210 |
| tryptophan for arg | TTCTCTTCCTCTGTGCGCCGGTCTCTCCCAGGACAGGCACAA | |
| nine, was found in a | ACACGCACCTCAAAGCTGTTCCGTCCCAGTAGATTACC | |
| patient who developed | GGAGAGACCGGCGCACA | 211 |
| osteosarcoma at the | TGTGCGCCGGTCTCTCC | 212 |
| age of 10 years. | | |
| In 5 of 6 anaplastic | GCTTCTCTTTTCCTATCCTGAGTAGTGGTAATCTACTGGGACG | 213 |
| carcinomas of the | GAACAGCTTTGAGGTGCGTGTTTGTGCCTGTCCTGGGAGAGA | |
| thyroid and in an | CCGGCGCACAGAGGAAGAGAATCTCCGCAAGAAAGG | |
| anaplastic carcinoma | CCTTTCTTGCGGAGATTCTCTTCCTCTGTGCGCCGGTCTCTC | 214 |
| thyroid cell line ARO, a | CCAGGACAGGCACAAACACGCACCTCAAAGCTGTTCCGTCCC | |
| CGT-to-CAT mutation | AGTAGATTACCACTACTCAGGATAGGAAAAGAGAAGC | |
| converted | TGAGGTGCGTGTTTGTG | 215 |
| arginine-273 to | CACAAACACGCACCTCA | 216 |
| histidine. | | |
| A germline | TCCTAGCACTGCCCAACAACACCAGCTCCTCTCCCCAGCCAA | 217 |
| GGA-to-GTA mutation | AGAAGAAACCACTGGATGGl AGAATATTTCACCCTTCAGGTACT | |
| resulting in a change | AAGTCTTGGGACCTCTTATCAAGTGGAAAGTTTCCA | |
| of | TGGAAACTTTCCACTTGATAAGAGGTCCCAAGACTTAGTACCT | 218 |
| glycine-325 to valine | GAAGGGTGAAATATTCTCCATCCAGTGGTTTCTTCTTTGGCTG | |
| was found in a patient | GGGAGAGGAGCTGGTGTTGTTGGGCAGTGCTAGGA | |
| who had non-Hodgkin | ACTGGATGGAGAATATT | 219 |
| lymphoma diagnosed | AATATTCTCCATCCAGT | 220 |
| at age 17 and colon | | |
| carcinoma at age 26. | | |
| CGC-CCC | AATGGTTCACTGAAGACCCAGGTCCAGATGAAGCTCCCAGAA | 221 |
| Arg-72 to Pro | TGCCAGAGGCTGCTCCCCGCGTGGCCCCTGCACCAGCAGCT | |
| association with Lung | CCTACACCGGCGGCCCCTGCACCAGCCCCCTCCTGGCC | |
| cancer | GGCCAGGAGGGGGCTGGTGCAGGGGCCGCCGGTGTAGGAG | 222 |
| | CTGCTGGTGCAGGGGCCACGCGGGGAGCAGCCTCTGGCATT | |
| | CTGGGAGCTTCATCTGGACCTGGGTCTTCAGTGAACCATT | |
| | TGCTCCCCGCGTGGCCC | 223 |
| | GGGCCACGCGGGGAGCA | 224 |
| CCG-CTG | AAGCTCCCAGAATGCCAGAGGCTGCTCCCCGCGTGGCCCCT | 225 |
| Pro-82 to Leu | GCACCAGCAGCTCCTACACCGGCGGCCCCTGCACCAGCCCC | |
| Breast cancer | CTCCTGGCCCCTGTCATCTTCTGTCCCTTCCCAGAAAAC | |
| | GTTTTCTGGGAAGGGACAGAAGATGACAGGGGCCAGGAGGG | 226 |
| | GGCTGGTGCAGGGGCCGCCGGTGTAGGAGCTGCTGGTGCA | |
| | GGGGCCACGCGGGGAGCAGCCTCTGGCATTCTGGGAGCTT | |
| | TCCTACACCGGCGGCCC | 227 |
| | GGGCCGCCGGTGTAGGA | 228 |
| cCAA-TAA | TTCAACTCTGTCTCCTTCCTCTTCCTACAGTACTCCCCTGCCC | 229 |
| Gln-136 to Term | TCAACAAGATGTTTTGCCAACTGGCCAAGACCTGCCCTGTGC | |
| Li-Fraumeni syndrome | AGCTGTGGGTTGATTCCACACCCCCGCCCGGCACCC | |
| | GGGTGCCGGGCGGGGGTGTGGAATCAACCCACAGCTGCACA | 230 |
| | GGGCAGGTCTTGGCCAGTTGGCAAAACATCTTGTTGAGGGCA | |
| | GGGGAGTACTGTAGGAAGAGGAAGGAGACAGAGTTGAA | |
| | TGTTTTGCCAACTGGCC | 231 |
| | GGCCAGTTGGCAAAACA | 232 |
| TGC-TAC | TCCTCTTCCTACAGTACTCCCCTGCCCTCAACAAGATGTTTTG | 233 |
| Cys-141 to Tyr | CCAACTGGCCAAGACCTGCCCTGTGCAGCTGTGGGTTGATTC | |
| Li-Fraumeni syndrome | CACACCCCCGCCCGGCACCCGCGTCCGCGCCATGGC | |
| | GCCATGGCGCGGACGCGGGTGCCGGGCGGGGGTGTGGAAT | 234 |
| | CAACCCACAGCTGCACAGGGCAGGTCTTGGCCAGTTGGCAA | |
| | AACATCTTGTTGAGGGCAGGGGAGTACTGTAGGAAGAGGA | |
| | CAAGACCTGCCCTGTGC | 235 |
| | GCACAGGGCAGGTCTTG | 236 |
| aCCC-TCC | AACAAGATGTTTTGCCAACTGGCCAAGACCTGCCCTGTGCAG | 237 |
| Pro-151 to Ser | CTGTGGGTTGATTCCACACCCCCGCCCGGCACCCGCGTCCG | |
| Li-Fraumeni syndrome | CGCCATGGCCATCTACAAGCAGTCACAGCACATGACGG | |
| | CCGTCATGTGCTGTGACTGCTTGTAGATGGCCATGGCGCGGA | 238 |
| | CGCGGGTGCCGGGCGGGGGTGTGGAATCAACCCACAGCTG | |
| | CACAGGGCAGGTCTTGGCCAGTTGGCAAAACATCTTGTT | |
| | ATTCCACACCCCCGCCC | 239 |
| | GGGCGGGGGTGTGGAAT | 240 |
| CCG-CTG | AGATGTTTTGCCAACTGGCCAAGACCTGCCCTGTGCAGCTGT | 241 |
| Pro-152 to Leu | GGGTTGATTCCACACCCCCGCCCGGCACCCGCGTCCGCGCC | |
| Adrenocortical | ATGGCCATCTACAAGCAGTCACAGCACATGACGGAGGT | |
| carcinoma | ACCTCCGTCATGTGCTGTGACTGCTTGTAGATGGCCATGGCG | 242 |
| | CGGACGCGGGTGCCGGGCGGGGGTGTGGAATCAACCCACA | |
| | GCTGCACAGGGCAGGTCTTGGCCAGTTGGCAAAACATCT | |
| | CACACCCCCGCCCGGCA | 243 |
| | TGCCGGGCGGGGGTGTG | 244 |
| GGC-GTC | TTTGCCAACTGGCCAAGACCTGCCCTGTGCAGCTGTGGGTTG | 245 |
| Gly-154 to Val | ATTCCACACCCCCGCCCGGCACCCGCGTCCGCGCCATGGCC | |
| Glioblastoma | ATCTACAAGCAGTCACAGCACATGACGGAGGTTGTGAG | |
| | CTCACAACCTCCGTCATGTGCTGTGACTGCTTGTAGATGGCC | 246 |
| | ATGGCGCGGACGCGGGTGCCGGGCGGGGGTGTGGAATCAA | |
| | CCCACAGCTGCACAGGGCAGGTCTTGGCCAGTTGGCAAA | |
| | CCCGCCCGGCACCCGCG | 247 |
| | CGCGGGTGCCGGGCGGG | 248 |
| CGC-CAC | CCCGCGTCCGCGCCATGGCCATCTACAAGCAGTCACAGCAC | 249 |
| Arg-175 to His | ATGACGGAGGTTGTGAGGCGCTGCCCCCACCATGAGCGCTG | |
| Li-Fraumeni syndrome | CTCAGATAGCGATGGTGAGCAGCTGGGGCTGGAGAGACG | |
| | CGTCTCTCCAGCCCCAGCTGCTCACCATCGCTATCTGAGCAG | 250 |
| | CGCTCATGGTGGGGGCAGCGCCTCACAACCTCCGTCATGTG | |
| | CTGTGACTGCTTGTAGATGGCCATGGCGCGGACGCGGG | |
| | TGTGAGGCGCTGCCCCC | 251 |
| | GGGGGCAGCGCCTCACA | 252 |
| tGAG-AAG | ATGGCCATCTACAAGCAGTCACAGCACATGACGGAGGTTGTG | 253 |
| Glu-180 to Lys | AGGCGCTGCCCCCACCATGAGCGCTGCTCAGATAGCGATGG | |
| Li-Fraumeni syndrome | TGAGCAGCTGGGGCTGGAGAGACGACAGGGCTGGTTGC | |
| | GCAACCAGCCCTGTCGTCTCTCCAGCCCCAGCTGCTCACCAT | 254 |
| | CGCTATCTGAGCAGCGCTCATGGTGGGGGCAGCGCCTCACA | |
| | ACCTCCGTCATGTGCTGTGACTGCTTGTAGATGGCCAT | |
| | CCCACCATGAGCGCTGC | 255 |
| | GCAGCGCTCATGGTGGG | 256 |
| gCGC-TGC | GCCATCTACAAGCAGTCACAGCACATGACGGAGGTTGTGAGG | 257 |
| Arg-181 to Cys | CGCTGCCCCCACCATGAGCGCTGCTCAGATAGCGATGGTGA | |
| Breast cancer | GCAGCTGGGGCTGGAGAGACGACAGGGCTGGTTGCCCA | |
| | TGGGCAACCAGCCCTGTCGTCTCTCCAGCCCCAGCTGCTCAC | 258 |
| | CATCGCTATCTGAGCAGCGCTCATGGTGGGGGCAGCGCCTC | |
| | ACAACCTCCGTCATGTGCTGTGACTGCTTGTAGATGGC | |
| | ACCATGAGCGCTGCTCA | 259 |
| | TGAGCAGCGCTCATGGT | 260 |
| CGC-CAC | CCATCTACAAGCAGTCACAGCACATGACGGAGGTTGTGAGGC | 261 |
| Arg-81 to His | GCTGCCCCCACCATGAGCGCTGCTCAGATAGCGATGGTGAG | |
| Breast cancer | CAGCTGGGGCTGGAGAGACGACAGGGCTGGTTGCCCAG | |
| | CTGGGCAACCAGCCCTGTCGTCTCTCCAGCCCCAGCTGCTCA | 262 |
| | CCATCGCTATCTGAGCAGCGCTCATGGTGGGGGCAGCGCCT | |
| | CACAACCTCCGTCATGTGCTGTGACTGCTTGTAGATGG | |
| | CCATGAGCGCTGCTCAG | 263 |
| | CTGAGCAGCGCTCATGG | 264 |
| CAT-CGT | CCAGGGTCCCCAGGCCTCTGATTCCTCACTGATTGCTCTTAG | 265 |
| His-193 to Arg | GTCTGGCCCCTCCTCAGCATCTTATCCGAGTGGAAGGAAATT | |
| Li-Fraumeni syndrome | TGCGTGTGGAGTATTTGGATGACAGAAACACTTTTCG | |
| | CGAAAAGTGTTTCTGTCATCCAAATACTCCACACGCAAATTTC | 266 |
| | CTTCCACTCGGATAAGATGCTGAGGAGGGGCCAGACCTAAGA | |
| | GCAATCAGTGAGGAATCAGAGGCCTGGGGACCCTGG | |
| | TCCTCAGCATCTTATCC | 267 |
| | GGATAAGATGCTGAGGA | 268 |
| cCGA-TGA | CCCAGGCCTCTGATTCCTCACTGATTGCTCTTAGGTCTGGCC | 269 |
| Arg-196 to Term | CCTCCTCAGCATCTTATCCGAGTGGAAGGAAATTTGCGTGTG | |
| Adrenocortical | GAGTATTTGGATGACAGAAACACTTTTCGACATAGTG | |
| carcinoma | CACTATGTCGAAAAGTGTTTCTGTCATCCAAATACTCCACACG | 270 |
| | CAAATTTCCTTCCACTCGGATAAGATGCTGAGGAGGGGCCAG | |
| | ACCTAAGAGCAATCAGTGAGGAATCAGAGGCCTGGG | |
| | ATCTTATCCGAGTGGAA | 271 |
| | TTCCACTCGGATAAGAT | 272 |
| cAGA-TGA | GCCCCTCCTCAGCATCTTATCCGAGTGGAAGGAAATTTGCGT | 273 |
| Arg-209 to Term | GTGGAGTATTTGGATGACAGAAACACTTTTCGACATAGTGTG | |
| Li-Fraumeni syndrome | GTGGTGCCCTATGAGCCGCCTGAGGTCTGGTTTGCAA | |
| | TTGCAAACCAGACCTCAGGCGGCTCATAGGGCACCACCACAC | 274 |
| | TATGTCGAAAAGTGTTTCTGTCATCCAAATACTCCACACGCAA | |
| | ATTTCCTTCCACTCGGATAAGATGCTGAGGAGGGGC | |
| | TGGATGACAGAAACACT | 275 |
| | AGTGTTTCTGTCATCCA | 276 |
| tCGA-TGA | CATCTTATCCGAGTGGAAGGAAATTTGCGTGTGGAGTATTTG | 277 |
| Arg-213 to Term | GATGACAGAAACACTTTTCGACATAGTGTGGTGGTGCCCTAT | |
| Li-Fraumeni syndrome | GAGCCGCCTGAGGTCTGGTTTGCAACTGGGGTCTCTG | |
| | CAGAGACCCCAGTTGCAAACCAGACCTCAGGCGGCTCATAG | 278 |
| | GGCACCACCACACTATGTCGAAAAGTGTTTCTGTCATCCAAAT | |
| | ACTCCACACGCAAATTTCCTTCCACTCGGATAAGATG | |
| | ACACTTTTCGACATAGT | 279 |
| | ACTATGTCGAAAAGTGT | 280 |
| gCCC-TCC | GGAAATTTGCGTGTGGAGTATTTGGATGACAGAAACACTTTTC | 281 |
| Pro-219 to Ser | GACATAGTGTGGTGGTGCCCTATGAGCCGCCTGAGGTCTGG | |
| Adrenocortical | TTTGCAACTGGGGTCTCTGGGAGGAGGGGTTAAGGGT | |
| carcinoma | ACCCTTAACCCCTCCTCCCAGAGACCCCAGTTGCAAACCAGA | 282 |
| | CCTCAGGCGGCTCATAGGGCACCACCACACTATGTCGAAAAG | |
| | TGTTTCTGTCATCCAAATACTCCACACGCAAATTTCC | |
| | TGGTGGTGCCCTATGAG | 283 |
| | CTCATAGGGCACCACCA | 284 |
| TAT-TGT | ATTTGCGTGTGGAGTATTTGGATGACAGAAACACTTTTCGACA | 285 |
| Tyr-220 to Cys | TAGTGTGGTGGTGCCCTATGAGCCGCCTGAGGTCTGGTTTGC | |
| Li-Fraumeni syndrome | AACTGGGGTCTCTGGGAGGAGGGGTTAAGGGTGGTT | |
| | AACCACCCTTAACCCCTCCTCCCAGAGACCCCAGTTGCAAAC | 286 |
| | CAGACCTCAGGCGGCTCATAGGGCACCACCACACTATGTCGA | |
| | AAAGTGTTTCTGTCATCCAAATACTCCACACGCAAAT | |
| | GGTGCCCTATGAGCCGC | 287 |
| | GCGGCTCATAGGGCACC | 288 |
| cTCT-ACT | CACAGGTCTCCCCAAGGCGCACTGGCCTCATCTTGGGCCTGT | 289 |
| Ser-227 to Thr | GTTATCTCCTAGGTTGGCTCTGACTGTACCACCATCCACTACA | |
| Rhabdomyosarcoma | ACTACATGTGTAACAGTTCCTGCATGGGCGGCATGA | |
| | TCATGCCGCCCATGCAGGAACTGTTACACATGTAGTTGTAGT | 290 |
| | GGATGGTGGTACAGTCAGAGCCAACCTAGGAGATAACACAG | |
| | GCCCAAGATGAGGCCAGTGCGCCTTGGGGAGACCTGTG | |
| | AGGTTGGCTCTGACTGT | 291 |
| | ACAGTCAGAGCCAACCT | 292 |
| cCAC-AAC | GCACTGGCCTCATCTTGGGCCTGTGTTATCTCCTAGGTTGGC | 293 |
| His-233 to Asn | TCTGACTGTACCACCATCCACTACAACTACATGTGTAACAGTT | |
| Glioma | CCTGCATGGGCGGCATGAACCGGAGGCCCATCCTCA | |
| | TGAGGATGGGCCTCCGGTTCATGCCGCCCATGCAGGAACTG | 294 |
| | TTACACATGTAGTTGTAGTGGATGGTGGTACAGTCAGAGCCA | |
| | ACCTAGGAGATAACACAGGCCCAAGATGAGGCCAGTGC | |
| | CCACCATCCACTACAAC | 295 |
| | GTTGTAGTGGATGGTGG | 296 |
| cAAC-GAC | GCCTCATCTTGGGCCTGTGTTATCTCCTAGGTTGGCTCTGAC | 297 |
| Asn-235 to Asp | TGTACCACCATCCACTACAACTACATGTGTAACAGTTCCTGCA | |
| Adrenocortical | TGGGCGGCATGAACCGGAGGCCCATCCTCACCATCA | |
| carcinoma | TGATGGTGAGGATGGGCCTCCGGTTCATGCCGCCCATGCAG | 298 |
| | GAACTGTTACACATGTAGTTGTAGTGGATGGTGGTACAGTCA | |
| | GAGCCAACCTAGGAGATAACACAGGCCCAAGATGAGGC | |
| | TCCACTACAACTACATG | 299 |
| | CATGTAGTTGTAGTGGA | 300 |
| AAC-AGC | CCTCATCTTGGGCCTGTGTTATCTCCTAGGTTGGCTCTGACT | 301 |
| Asn-235 to Ser | GTACCACCATCCACTACAACTACATGTGTAACAGTTCCTGCAT | |
| Rhabdomyosarcoma | GGGCGGCATGAACCGGAGGCCCATCCTCACCATCAT | |
| | ATGATGGTGAGGATGGGCCTCCGGTTCATGCCGCCCATGCA | 302 |
| | GGAACTGTTACACATGTAGTTGTAGTGGATGGTGGTACAGTC | |
| | AGAGCCAACCTAGGAGATAACACAGGCCCAAGATGAGG | |
| | CCACTACAACTACATGT | 303 |
| | ACATGTAGTTGTAGTGG | 304 |
| ATCc-ATG | CATCCACTACAACTACATGTGTAACAGTTCCTGCATGGGCGG | 305 |
| Ile-251 to Met | CATGAACCGGAGGCCCATCCTCACCATCATCACACTGGAAGA | |
| Glioma | CTCCAGGTCAGGAGCCACTTGCCACCCTGCACACTGG | |
| | CCAGTGTGCAGGGTGGCAAGTGGCTCCTGACCTGGAGTCTT | 306 |
| | CCAGTGTGATGATGGTGAGGATGGGCCTCCGGTTCATGCCG | |
| | CCCATGCAGGAACTGTTACACATGTAGTTGTAGTGGATG | |
| | AGGCCCATCCTCACCAT | 307 |
| | ATGGTGAGGATGGGCCT | 308 |
| ACA-ATA | ACATGTGTAACAGTTCCTGCATGGGCGGCATGAACCGGAGG | 309 |
| Thr-256 to Ile | CCCATCCTCACCATCATCACACTGGAAGACTCCAGGTCAGGA | |
| Glioblastoma | GCCACTTGCCACCCTGCACACTGGCCTGCTGTGCCCCA | |
| | TGGGGCACAGCAGGCCAGTGTGCAGGGTGGCAAGTGGCTCC | 310 |
| | TGACCTGGAGTCTTCCAGTGTGATGATGGTGAGGATGGGCCT | |
| | CCGGTTCATGCCGCCCATGCAGGAACTGTTACACATGT | |
| | CATCATCACACTGGAAG | 311 |
| | CTTCCAGTGTGATGATG | 312 |
| CTG-CAG | TGTGTAACAGTTCCTGCATGGGCGGCATGAACCGGAGGCCC | 313 |
| Leu-257 to Gln | ATCCTCACCATCATCACACTGGAAGACTCCAGGTCAGGAGCC | |
| Li-Fraumeni syndrome | ACTTGCCACCCTGCACACTGGCCTGCTGTGCCCCAGCC | |
| | GGCTGGGGCACAGCAGGCCAGTGTGCAGGGTGGCAAGTGG | 314 |
| | CTCCTGACCTGGAGTCTTCCAGTGTGATGATGGTGAGGATGG | |
| | GCCTCCGGTTCATGCCGCCCATGCAGGAACTGTTACACA | |
| | CATCACACTGGAAGACT | 315 |
| | AGTCTTCCAGTGTGATG | 316 |
| CTG-CCG | GACCTGATTTCCTTACTGCCTCTTGCTTCTCTTTTCCTATCCTG | 317 |
| Leu-265 to Pro | AGTAGTGGTAATCTACTGGGACGGAACAGCTTTGAGGTGCGT | |
| Li-Fraumeni syndrome | GTTTGTGCCTGTCCTGGGAGAGACCGGCGCACAGA | |
| | TCTGTGCGCCGGTCTCTCCCAGGACAGGCACAAACACGCAC | 318 |
| | CTCAAAGCTGTTCCGTCCCAGTAGATTACCACTACTCAGGATA | |
| | GGAAAAGAGAAGCAAGAGGCAGTAAGGAAATCAGGTC | |
| | TAATCTACTGGGACGGA | 319 |
| | TCCGTCCCAGTAGATTA | 320 |
| gCGT-TGT | TGCTTCTCTTTTCCTATCCTGAGTAGTGGTAATCTACTGGGAC | 321 |
| Arg-273 to Cys | GGAACAGCTTTGAGGTGCGTGTTTGTGCCTGTCCTGGGAGAG | |
| Li-Fraumeni syndrome | ACCGGCGCACAGAGGAAGAGAATCTCCGCAAGAAAG | |
| | CTTTCTTGCGGAGATTCTCTTCCTCTGTGCGCCGGTCTCTCC | 322 |
| | CAGGACAGGCACAAACACGCACCTCAAAGCTGTTCCGTCCCA | |
| | GTAGATTACCACTACTCAGGATAGGAAAAGAGAAGCA | |
| | TTGAGGTGCGTGTTTGT | 323 |
| | ACAAACACGCACCTCAA | 324 |
| TGT-TAT | CTTTTCCTATCCTGAGTAGTGGTAATCTACTGGGACGGAACA | 325 |
| Cys-275 to Tyr | GCTTTGAGGTGCGTGTTTGTGCCTGTCCTGGGAGAGACCGG | |
| Li-Fraumeni syndrome | CGCACAGAGGAAGAGAATCTCCGCAAGAAAGGGGAGCC | |
| | GGCTCCCCTTTCTTGCGGAGATTCTCTTCCTCTGTGCGCCGG | 326 |
| | TCTCTCCCAGGACAGGCACAAACACGCACCTCAAAGCTGTTC | |
| | CGTCCCAGTAGATTACCACTACTCAGGATAGGAAAAG | |
| | GCGTGTTTGTGCCTGTC | 327 |
| | GACAGGCACAAACACGC | 328 |
| CCT-CTT | TCCTGAGTAGTGGTAATCTACTGGGACGGAACAGCTTTGAGG | 329 |
| Pro-278 to Leu | TGCGTGTTTGTGCCTGTCCTGGGAGAGACCGGCGCACAGAG | |
| Breast cancer | GAAGAGAATCTCCGCAAGAAAGGGGAGCCTCACCACGA | |
| | TCGTGGTGAGGCTCCCCTTTCTTGCGGAGATTCTCTTCCTCT | 330 |
| | GTGCGCCGGTCTCTCCCAGGACAGGCACAAACACGCACCTC | |
| | AAAGCTGTTCCGTCCCAGTAGATTACCACTACTCAGGA | |
| | TGCCTGTCCTGGGAGAG | 331 |
| | CTCTCCCAGGACAGGCA | 332 |
| AGA-AAA | GTAGTGGTAATCTACTGGGACGGAACAGCTTTGAGGTGCGTG | 333 |
| Arg-280 to Lys | TTTGTGCCTGTCCTGGGAGAGACCGGCGCACAGAGGAAGAG | |
| Glioma | AATCTCCGCAAGAAAGGGGAGCCTCACCACGAGCTGCC | |
| | GGCAGCTCGTGGTGAGGCTCCCCTTTCTTGCGGAGATTCTCT | 334 |
| | TCCTCTGTGCGCCGGTCTCTCCCAGGACAGGCACAAACACG | |
| | CACCTCAAAGCTGTTCCGTCCCAGTAGATTACCACTAC | |
| | TCCTGGGAGAGACCGGC | 335 |
| | GCCGGTCTCTCCCAGGA | 336 |
| GAA-GCA | GGAACAGCTTTGAGGTGCGTGTTTGTGCCTGTCCTGGGAGAG | 337 |
| Glu-286 to Ala | ACCGGCGCACAGAGGAAGAGAATCTCCGCAAGAAAGGGGAG | |
| Adrenocortical | CCTCACCACGAGCTGCCCCCAGGGAGCACTAAGCGAGG | |
| carcinoma | CCTCGCTTAGTGCTCCCTGGGGGCAGCTCGTGGTGAGGCTC | 338 |
| | CCCTTTCTTGCGGAGATTCTCTTCCTCTGTGCGCCGGTCTCT | |
| | CCCAGGACAGGCACAAACACGCACCTCAAAGCTGTTCC | |
| | AGAGGAAGAGAATCTCC | 339 |
| | GGAGATTCTCTTCCTCT | 340 |
| CGA-CCA | AAGAGAATCTCCGCAAGAAAGGGGAGCCTCACCACGAGCTG | 341 |
| Arg-306 to Pro | CCCCCAGGGAGCACTAAGCGAGGTAAGCAAGCAGGACAAGA | |
| Rhabdomyosarcoma | AGCGGTGGAGGAGACCAAGGGTGCAGTTATGCCTCAGAT | |
| | ATCTGAGGCATAACTGCACCCTTGGTCTCCTCCACCGCTTCTT | 342 |
| | GTCCTGCTTGCTTACCTCGCTTAGTGCTCCCTGGGGGCAGCT | |
| | CGTGGTGAGGCTCCCCTTTCTTGCGGAGATTCTCTT | |
| | CACTAAGCGAGGTAAGC | 343 |
| | GCTTACCTCGCTTAGTG | 344 |
| gCGA-TGA | GAAGAGAATCTCCGCAAGAAAGGGGAGCCTCACCACGAGCT | 345 |
| Arg-306 to Term | GCCCCCAGGGAGCACTAAGCGAGGTAAGCAAGCAGGACAAG | |
| Li-Fraumeni syndrome | AAGCGGTGGAGGAGACCAAGGGTGCAGTTATGCCTCAGA | |
| | TCTGAGGCATAACTGCACCCTTGGTCTCCTCCACCGCTTCTT | 346 |
| | GTCCTGCTTGCTTACCTCGCTTAGTGCTCCCTGGGGGCAGCT | |
| | CGTGGTGAGGCTCCCCTTTCTTGCGGAGATTCTCTTC | |
| | GCACTAAGCGAGGTAAG | 347 |
| | CTTACCTCGCTTAGTGC | 348 |
| gCGC-TGC | GGTACTGTGAATATACTTACTTCTCCCCCTCCTCTGTTGCTGC | 349 |
| Arg-337 to Cys | AGATCCGTGGGCGTGAGCGCTTCGAGATGTTCCGAGAGCTG | |
| Osteosarcoma | AATGAGGCCTTGGAACTCAAGGATGCCCAGGCTGGGA | |
| | TCCCAGCCTGGGCATCCTTGAGTTCCAAGGCCTCATTCAGCT | 350 |
| | CTCGGAACATCTCGAAGCGCTCACGCCCACGGATCTGCAGC | |
| | AACAGAGGAGGGGGAGAAGTAAGTATATTCACAGTACC | |
| | GGCGTGAGCGCTTCGAG | 351 |
| | CTCGAAGCGCTCACGCC | 352 |
| CTG-CCG | CTCCCCCTCCTCTGTTGCTGCAGATCCGTGGGCGTGAGCGCT | 353 |
| Leu-344 to Pro | TCGAGATGTTCCGAGAGCTGAATGAGGCCTTGGAACTCAAGG | |
| Li-Fraumeni syndrome | ATGCCCAGGCTGGGAAGGAGCCAGGGGGGAGCAGGGC | |
| | GCCCTGCTCCCCCCTGGCTCCTTCCCAGCCTGGGCATCCTTG | 354 |
| | AGTTCCAAGGCCTCATTCAGCTCTCGGAACATCTCGAAGCGC | |
| | TCACGCCCACGGATCTGCAGCAACAGAGGAGGGGGAG | |
| | CCGAGAGCTGAATGAGG | 355 |
| | CCTCATTCAGCTCTCGG | 356 |

### EXAMPLE 7

### Beta Globin

Hemoglobin, the major protein in the red blood cell, binds oxygen reversibly and is responsible for the cells' capacity to transport oxygen to the tissues. In adults, the major hemoglobin is hemoglobin A, a tetrameric protein consisting of two identical alpha globin chains and two beta globin chains. Disorders involving hemoglobin are among the most common genetic disorders worldwide, with approximately 5% of the world's population being carriers for clinically important hemoglobin mutations. Approximately 300,000 severely affected homozygotes or compound heterozygotes are born each year.

Mutation of the glutamic acid at position 7 in beta globin to valine causes sickle cell anemia, the clinical manifestations of which are well known. Mutations that cause absence of beta chain cause beta-zero-thalassemia. Reduced amounts of detectable beta globin causes beta-plus-thalassemia. For clinical purposes, beta-thalassemia is divided into thalassemia major (transfusion dependent), thalassemia intermedia (of intermediate severity), and thalassemia minor (asymptomatic). Patients with thalassemia major present in the first year of life with severe anemia; they are unable to maintain a hemoglobin level about 5 gm/dl.

The beta-thalassemias were among the first human genetic diseases to be examined by means of recombinant DNA analysis. Baysal et al., Hemoglobin 19(3-4):213-36 (1995) and others provide a compendium of mutations that result in beta-thalassemia.

Hemoglobin disorders were among the first to be considered for gene therapy. Transcriptional silencing of genes transferred into hematopoietic stem cells, however, poses one of the most significant challenges to its success. If the transferred gene is not completely silenced, a progressive decline in gene expression is often observed. Position effect variegation (PEV) and silencing mechanisms may act on a transferred globin gene residing in chromatin outside of the normal globin locus during the important terminal phases of erythroblast development when globin transcripts normally accumulate rapidly despite heterochromatization and shutdown of the rest of the genome. The attached table discloses the correcting oligonucleotide base sequences for the beta globin oligonucleotides of the invention.

TABLE 8
Beta Globin Mutations and Genome-Correcting Oligos

| | | |
|---|---|---|
| Clinical Phenotype & | | SEQ ID |
| Mutation | Correcting Oligos | NO: |

| | | |
|---|---|---|
| Sickle Cell Anemia | TCTGACACAACTGTGTTCACTAGCAACCTCAAACAGACACCA | 357 |
| GLU-7-VAL | TGGTGCACCTGACTCCTGAGGAGAAGTCTGCCGTTACTGCC | |
| GAG to GTG | CTGTGGGGCAAGGTGAACGTGGATGAAGTTGGTGGTGA | |
| | TCACCACCAACTTCATCCACGTTCACCTTGCCCCACAGGGCA | 358 |
| | GTAACGGCAGACTTCTCCTCAGGAGTCAGGTGCACCATGGT | |
| | GTCTGTTTGAGGTTGCTAGTGAACACAGTTGTGTCAGA | |
| | GACTCCTGAGGAGAAGT | 359 |
| | ACTTCTCCTCAGGAGTC | 360 |
| Thalassaemia Beta | CTATTGCTTACATTTGCTTCTGACACAACTGTGTTCACTAGCA | 361 |
| MET-0-ARG | ACCTCAAACAGACACCATGGTGCACCTGACTCCTGAGGAGA | |
| ATG to AGG | AGTCTGCCGTTACTGCCCTGTGGGGCAAGGTGAACGT | |
| | ACGTTCACCTTGCCCCACAGGGCAGTAACGGCAGACTTCTC | 362 |
| | CTCAGGAGTCAGGTGCACCATGGTGTCTGTTTGAGGTTGCTA | |
| | GTGAACACAGTTGTGTCAGAAGCAAATGTAAGCAATAG | |
| | AGACACCATGGTGCACC | 363 |
| | GGTGCACCATGGTGTCT | 364 |
| Thalassaemia Beta | TATTGCTTACATTTGCTTCTGACACAACTGTGTTCACTAGCAA | 365 |
| MET-0-ILE | CCTCAAACAGACACCATGGTGCACCTGACTCCTGAGGAGAA | |
| ATG to ATA | GTCTGCCGTTACTGCCCTGTGGGGCAAGGTGAACGTG | |
| | CACGTTCACCTTGCCCCACAGGGCAGTAACGGCAGACTTCT | 366 |
| | CCTCAGGAGTCAGGTGCACCATGGTGTCTGTTTGAGGTTGCT | |
| | AGTGAACACAGTTGTGTCAGAAGCAAATGTAAGCAATA | |
| | GACACCATGGTGCACCT | 367 |
| | AGGTGCACCATGGTGTC | 368 |
| Thalassaemia Beta | TATTGCTTACATTTGCTTCTGACACAACTGTGTTCACTAGCAA | 369 |
| MET-0-ILE | CCTCAAACAGACACCATGGTGCACCTGACTCCTGAGGAGAA | |
| ATG to ATT | GTCTGCCGTTACTGCCCTGTGGGGCAAGGTGAACGTG | |
| | CACGTTCACCTTGCCCCACAGGGCAGTAACGGCAGACTTCT | 370 |
| | CCTCAGGAGTCAGGTGCACCATGGTGTCTGTTTGAGGTTGCT | |
| | AGTGAACACAGTTGTGTCAGAAGCAAATGTAAGCAATA | |
| | GACACCATGGTGCACCT | 371 |
| | AGGTGCACCATGGTGTC | 372 |
| Thalassaemia Beta | CTATTGCTTACATTTGCTTCTGACACAACTGTGTTCACTAGCA | 373 |
| MET-0-LYS | ACCTCAAACAGACACCATGGTGCACCTGACTCCTGAGGAGA | |
| ATG to AAG | AGTCTGCCGTTACTGCCCTGTGGGGCAAGGTGAACGT | |
| | ACGTTCACCTTGCCCCACAGGGCAGTAACGGCAGACTTCTC | 374 |
| | CTCAGGAGTCAGGTGCACCATGGTGTCTGTTTGAGGTTGCTA | |
| | GTGAACACAGTTGTGTCAGAAGCAAATGTAAGCAATAG | |
| | AGACACCATGGTGCACC | 375 |
| | GGTGCACCATGGTGTCT | 376 |
| Thalassaemia Beta | CTATTGCTTACATTTGCTTCTGACACAACTGTGTTCACTAGCA | 377 |
| MET-0-THR | ACCTCAAACAGACACCATGGTGCACCTGACTCCTGAGGAGA | |
| ATG to ACG | AGTCTGCCGTTACTGCCCTGTGGGGCAAGGTGAACGT | |
| | ACGTTCACCTTGCCCCACAGGGCAGTAACGGCAGACTTCTC | 378 |
| | CTCAGGAGTCAGGTGCACCATGGTGTCTGTTTGAGGTTGCTA | |
| | GTGAACACAGTTGTGTCAGAAGCAAATGTAAGCAATAG | |
| | AGACACCATGGTGCACC | 379 |
| | GGTGCACCATGGTGTCT | 380 |
| Thalassaemia Beta | TCTATTGCTTACATTTGCTTCTGACACAACTGTGTTCACTAGC | 381 |
| MET-0-VAL | AACCTCAAACAGACACCATGGTGCACCTGACTCCTGAGGAG | |
| ATG to GTG | AAGTCTGCCGTTACTGCCCTGTGGGGCAAGGTGAACG | |
| | CGTTCACCTTGCCCCACAGGGCAGTAACGGCAGACTTCTCC | 382 |
| | TCAGGAGTCAGGTGCACCATGGTGTCTGTTTGAGGTTGCTAG | |
| | TGAACACAGTTGTGTCAGAAGCAAATGTAAGCAATAGA | |
| | CAGACACCATGGTGGAC | 383 |
| | GTGCACCATGGTGTCTG | 384 |
| Thalassaemia Beta | TCAAACAGACACCATGGTGCACCTGACTCCTGAGGAGAAGT | 385 |
| TRP-16-Term | CTGCCGTTACTGCCCTGTGGGGCAAGGTGAACGTGGATGAA | |
| TGG to TGA | GTTGGTGGTGAGGCCCTGGGCAGGTTGGTATCAAGGTTA | |
| | TAACCTTGATACCAACCTGCCCAGGGCCTCACCACCAACTTC | 386 |
| | ATCCACGTTCACCTTGCCCCACAGGGCAGTAACGGCAGACT | |
| | TCTCCTCAGGAGTCAGGTGCACCATGGTGTCTGTTTGA | |
| | GCCCTGTGGGGCAAGGT | 387 |
| | ACCTTGCCCCACAGGGC | 388 |
| Thalassaemia Beta | CTCAAACAGACACCATGGTGCACCTGACTCCTGAGGAGAAG | 389 |
| TRP-16-Term | TCTGCCGTTACTGCCCTGTGGGGCAAGGTGAACGTGGATGA | |
| TGG to TAG | AGTTGGTGGTGAGGCCCTGGGCAGGTTGGTATCAAGGTT | |
| | AACCTTGATACCAACCTGCCCAGGGCCTCACCACCAACTTCA | 390 |
| | TCCACGTTCACCTTGCCCCACAGGGCAGTAACGGCAGACTT | |
| | CTCCTCAGGAGTCAGGTGCACCATGGTGTCTGTTTGAG | |
| | TGCCCTGTGGGGCAAGG | 391 |
| | CCTTGCCCCACAGGGCA | 392 |
| Thalassaemia Beta | ACAGACACCATGGTGCACCTGACTCCTGAGGAGAAGTCTGC | 393 |
| LYS-18-Term | CGTTACTGCCCTGTGGGGCAAGGTGAACGTGGATGAAGTTG | |
| AAG to TAG | GTGGTGAGGCCCTGGGCAGGTTGGTATCAAGGTTACAAG | |
| | CTTGTAACCTTGATACCAACCTGCCCAGGGCCTCACCACCAA | 394 |
| | CTTCATCCACGTTCACCTTGCCCCACAGGGCAGTAACGGCA | |
| | GACTTCTCCTCAGGAGTCAGGTGCACCATGGTGTCTGT | |
| | TGTGGGGCAAGGTGAAC | 395 |
| | GTTCACCTTGCCCCACA | 396 |
| Thalassaemia Beta | CCATGGTGCACCTGACTCCTGAGGAGAAGTCTGCCGTTACT | 397 |
| ASN-20-SER | GCCCTGTGGGGCAAGGTGAACGTGGATGAAGTTGGTGGTGA | |
| AAC to AGC | GGCCCTGGGCAGGTTGGTATCAAGGTTACAAGACAGGTT | |
| | AACCTGTCTTGTAACCTTGATACCAACCTGCCCAGGGCCTCA | 398 |
| | CCACCAACTTCATCCACGTTCACCTTGCCCCACAGGGCAGTA | |
| | ACGGCAGACTTCTCCTCAGGAGTCAGGTGCACCATGG | |
| | CAAGGTGAACGTGGATG | 399 |
| | CATCCACGTTCACCTTG | 400 |
| Thalassaemia Beta | ACCTGACTCCTGAGGAGAAGTCTGCCGTTACTGCCCTGTGG | 401 |
| GLU-23-ALA | GGCAAGGTGAACGTGGATGAAGTTGGTGGTGAGGCCCTGG | |
| GAA to GCA | GCAGGTTGGTATCAAGGTTACAAGACAGGTTTAAGGAGAC | |
| | GTCTCCTTAAACCTGTCTTGTAACCTTGATACCAACCTGCCCA | 402 |
| | GGGCCTCACCACCAACTTCATCCACGTTCACCTTGCCCCACA | |
| | GGGCAGTAACGGCAGACTTCTCCTCAGGAGTCAGGT | |
| | CGTGGATGAAGTTGGTG | 403 |
| | CACCAACTTCATCCACG | 404 |
| Thalassaemia Beta | CACCTGACTCCTGAGGAGAAGTCTGCCGTTACTGCCCTGTG | 405 |
| GLU-23-term | GGGCAAGGTGAACGTGGATGAAGTTGGTGGTGAGGCCCTG | |
| GAA to TAA | GGCAGGTTGGTATCAAGGTTACAAGACAGGTTTAAGGAGA | |
| | TCTCCTTAAACCTGTCTTGTAACCTTGATACCAACCTGCCCAG | 406 |
| | GGCCTCACCACCAACTTCATCCACGTTCACCTTGCCCCACAG | |
| | GGCAGTAACGGCAGACTTCTCCTCAGGAGTCAGGTG | |
| | ACGTGGATGAAGTTGGT | 407 |
| | ACCAACTTCATCCACGT | 408 |
| Thalassaemia Beta | GAGGAGAAGACTGCTGTCAATGCCCTGTGGGGCAAAGTGAA | 409 |
| GLU-27-LYS | CGTGGATGCAGTTGGTGGTGAGGCCCTGGGCAGGTTGGTAT | |
| GAG to AAG | CAAGGTTATAAGAGAGGCTCAAGGAGGCAAATGGAAACT | |
| | AGTTTCCATTTGCCTCCTTGAGCCTCTCTTATAACCTTGATAC | 410 |
| | CAACCTGCCCAGGGCCTCACCACCAACTGCATCCACGTTCA | |
| | CTTTGCCCCACAGGGCATTGACAGCAGTCTTCTCCTC | |
| | TTGGTGGTGAGGCCCTG | 411 |
| | CAGGGCCTCACCACCAA | 412 |
| Thalassaemia Beta | GAGGAGAAGACTGCTGTCAATGCCCTGTGGGGCAAAGTGAA | 413 |
| GLU-27-Term | CGTGGATGCAGTTGGTGGTGAGGCCCTGGGCAGGTTGGTAT | |
| GAG to TAG | CAAGGTTATAAGAGAGGCTCAAGGAGGCAAATGGAAACT | |
| | AGTTTCCATTTGCCTCCTTGAGCCTCTCTTATAACCTTGATAC | 414 |
| | CAACCTGCCCAGGGCCTCACCACCAACTGCATCCACGTTCA | |
| | CTTTGCCCCACAGGGCATTGACAGCAGTCTTCTCCTC | |
| | TTGGTGGTGAGGCCCTG | 415 |
| | CAGGGCCTCACCACCAA | 416 |
| Thalassaemia Beta | GAGAAGACTGCTGTCAATGCCCTGTGGGGCAAAGTGAACGT | 417 |
| ALA-28-SER | GGATGCAGTTGGTGGTGAGGCCCTGGGCAGGTTGGTATCAA | |
| GCC to TCC | GGTTATAAGAGAGGCTCAAGGAGGCAAATGGAAACTGGG | |
| | CCCAGTTTCCATTTGCCTCCTTGAGCCTCTCTTATAACCTTGA | 418 |
| | TACCAACCTGCCCAGGGCCTCACCACCAACTGCATCCACGTT | |
| | CACTTTGCCCCACAGGGCATTGACAGCAGTCTTCTC | |
| | GTGGTGAGGCCCTGGGC | 419 |
| | GCCCAGGGCCTCACCAC | 420 |
| Thalassaemia Beta | CTGTCAATGCCCTGTGGGGCAAAGTGAACGTGGATGCAGTT | 421 |
| ARG-31-THR | GGTGGTGAGGCCCTGGGCAGGTTGGTATCAAGGTTATAAGA | |
| AGG to ACG | GAGGCTCAAGGAGGCAAATGGAAACTGGGCATGTGTAGA | |
| | TCTACACATGCCCAGTTTCCATTTGCCTCCTTGAGCCTCTCTT | 422 |
| | ATAACCTTGATACCAACCTGCCCAGGGCCTCACCACCAACTG | |
| | CATCCACGTTCACTTTGCCCCACAGGGCATTGACAG | |
| | CCTGGGCAGGTTGGTAT | 423 |
| | ATACCAACCTGCCCAGG | 424 |
| Thalassaemia Beta | TGGGTTTCTGATAGGCACTGACTCTCTGTCCCTTGGGCTGTT | 425 |
| Leu-33-GLN | TTCCTACCCTCAGATTACTGGTGGTCTACCCTTGGACCCAGA | |
| CTG to CAG | GGTTCTTTGAGTCCTTTGGGGATCTGTCCTCTCCTGA | |
| | TCAGGAGAGGACAGATCCCCAAAGGACTCAAAGAACCTCTG | 426 |
| | GGTCCAAGGGTAGACCACCAGTAATCTGAGGGTAGGAAAAC | |
| | AGCCCAAGGGACAGAGAGTCAGTGCCTATCAGAAACCCA | |
| | CAGATTACTGGTGGTCT | 427 |
| | AGACCACCAGTAATCTG | 428 |
| Thalassaemia Beta | ATAGGCACTGACTCTCTGTCCCTTGGGCTGTTTTCCTACCCT | 429 |
| TYR-36-Term | CAGATTACTGGTGGTCTACCCTTGGACCCAGAGGTTCTTTGA | |
| TAC to TAA | GTCCTTTGGGGATCTGTCCTCTCCTGATGCTGTTATG | |
| | CATAACAGCATCAGGAGAGGACAGATCCCCAAAGGACTCAA | 430 |
| | AGAACCTCTGGGTCCAAGGGTAGACCACCAGTAATCTGAGG | |
| | GTAGGAAAACAGCCCAAGGGACAGAGAGTCAGTGCCTAT | |
| | GTGGTCTACCCTTGGAC | 431 |
| | GTCCAAGGGTAGACCAC | 432 |
| Thalassaemia Beta | ACTGACTCTCTGTCCCTTGGGCTGTTTTCCTACCCTCAGATTA | 433 |
| TRP-38-Term | CTGGTGGTCTACCCTTGGACCCAGAGGTTCTTTGAGTCCTTT | |
| TGG to TGA | GGGGATCTGTCCTCTCCTGATGCTGTTATGGGCAAC | |
| | GTTGCCCATAACAGCATCAGGAGAGGACAGATCCCCAAAGG | 434 |
| | ACTCAAAGAACCTCTGGGTCCAAGGGTAGACCACCAGTAATC | |
| | TGAGGGTAGGAAAACAGCCCAAGGGACAGAGAGTCAGT | |
| | TACCCTTGGACCCAGAG | 435 |
| | CTCTGGGTCCAAGGGTA | 436 |
| Thalassaemia Beta | CACTGACTCTCTGTCCCTTGGGCTGTTTTCCTACCCTCAGAT | 437 |
| TRP-38-Term | TACTGGTGGTCTACCCTTGGACCCAGAGGTTCTTTGAGTCCT | |
| TGG to TAG | TTGGGGATCTGTCCTCTCCTGATGCTGTTATGGGCAA | |
| | TTGCCCATAACAGCATCAGGAGAGGACAGATCCCCAAAGGA | 438 |
| | CTCAAAGAACCTCTGGGTCCAAGGGTAGACCACCAGTAATCT | |
| | GAGGGTAGGAAAACAGCCCAAGGGACAGAGAGTCAGTG | |
| | CTACCCTTGGACCCAGA | 439 |
| | TCTGGGTCCAAGGGTAG | 440 |
| Thalassaemia Beta | ACTCTCTGTCCCTTGGGCTGTTTTCCTACCCTCAGATTACTG | 441 |
| GLN-40-Term | GTGGTCTACCCTTGGACCCAGAGGTTCTTTGAGTCCTTTGGG | |
| CAG-TAG | GATCTGTCCTCTCCTGATGCTGTTATGGGCAACCCTA | |
| | TAGGGTTGCCCATAACAGCATCAGGAGAGGACAGATCCCCA | 442 |
| | AAGGACTCAAAGAACCTCTGGGTCCAAGGGTAGACCACCAG | |
| | TAATCTGAGGGTAGGAAAACAGCCCAAGGGACAGAGAGT | |
| | CTTGGACCCAGAGGTTC | 443 |
| | GAACCTCTGGGTCCAAG | 444 |
| Thalassaemia Beta | TTGGGCTGTTTTCCTACCCTCAGATTACTGGTGGTCTACCCT | 445 |
| GLU-44-Term | TGGACCCAGAGGTTCTTTGAGTCCTTTGGGGATCTGTCCTCT | |
| GAG to TAG | CCTGATGCTGTTATGGGCAACCCTAAGGTGAAGGCTC | |
| | GAGCCTTCACCTTAGGGTTGCCCATAACAGCATCAGGAGAG | 446 |
| | GACAGATCCCCAAAGGACTCAAAGAACCTCTGGGTCCAAGG | |
| | GTAGACCACCAGTAATCTGAGGGTAGGAAAACAGCCCAA | |
| | GGTTCTTTGAGTCCTTT | 447 |
| | AAAGGACTCAAAGAACC | 448 |
| Thalassaemia Beta | TTCTTTGAGTCCTTTGGGGATCTGTCCTCTCCTGATGCTGTTA | 449 |
| LYS-62-Term | TGGGCAACCCTAAGGTGAAGGCTCATGGCAAGAAGGTGCTA | |
| AAG to TAG | GGTGCCTTTAGTGATGGCCTGGCTCACCTGGACAACC | |
| | GGTTGTCCAGGTGAGCCAGGCCATCACTAAAGGCACCTAGC | 450 |
| | ACCTTCTTGCCATGAGCCTTCACCTTAGGGTTGCCCATAACA | |
| | GCATCAGGAGAGGACAGATCCCCAAAGGACTCAAAGAA | |
| | CTAAGGTGAAGGCTCAT | 451 |
| | ATGAGCCTTCACCTTAG | 452 |
| Thalassaemia Beta | TGCTGTTATGGGCAACCCTAAGGTGAAGGCTCATGGCAAGA | 453 |
| SER-73-ARG | AGGTGCTAGGTGCCTTTAGTGATGGCCTGGCTCACCTGGAC | |
| AGT to AGA | AACCTCAAGGGCACTTTTTCTCAGCTGAGTGAGCTGCAC | |
| | GTGCAGCTCACTCAGCTGAGAAAAAGTGCCCTTGAGGTTGTC | 454 |
| | CAGGTGAGCCAGGCCATCACTAAAGGCACCTAGCACCTTCT | |
| | TGCCATGAGCCTTCACCTTAGGGTTGCCCATAACAGCA | |
| | GCCTTTAGTGATGGCCT | 455 |
| | AGGCCATCACTAAAGGC | 456 |
| Haemolytic Anaemia | TTATGGGCAACCCTAAGGTGAAGGCTCATGGCAAGAAGGTG | 457 |
| GLY-75-VAL | CTAGGTGCCTTTAGTGATGGCCTGGCTCACCTGGACAACCTC | |
| GGC to GTC | AAGGGCACTTTTTCTCAGCTGAGTGAGCTGCACTGTGA | |
| | TCACAGTGCAGCTCACTCAGCTGAGAAAAAGTGCCCTTGAG | 458 |
| | GTTGTCCAGGTGAGCCAGGCCATCACTAAAGGCACCTAGCA | |
| | CCTTCTTGCCATGAGCCTTCACCTTAGGGTTGCCCATAA | |
| | TAGTGATGGCCTGGCTC | 459 |
| | GAGCCAGGCCATCACTA | 460 |
| Thalassaemia Beta | GCCTTTAGTGATGGCCTGGCTCACCTGGACAACCTCAAGGG | 461 |
| GLU-91-Term | CACCTTTGCCACACTGAGTGAGCTGCACTGTGACAAGCTGCA | |
| GAG to TAG | CGTGGATCCTGAGAACTTCAGGGTGAGTCTATGGGACC | |
| | GGTCCCATAGACTCACCCTGAAGTTCTCAGGATCCACGTGCA | 462 |
| | GCTTGTCACAGTGCAGCTCACTCAGTGTGGCAAAGGTGCCC | |
| | TTGAGGTTGTCCAGGTGAGCCAGGCCATCACTAAAGGC | |
| | CACTGAGTGAGCTGCAC | 463 |
| | GTGCAGCTCACTCAGTG | 464 |
| Thalassaemia Beta | CTGGACAACCTCAAGGGCACTTTTTCTCAGCTGAGTGAGCTG | 465 |
| VAL-99-MET | CACTGTGACAAGCTGCACGTGGATCCTGAGAACTTCAGGGT | |
| GTG to ATG | GAGTCCAGGAGATGCTTCACTTTTCTCTTTTTACTTTC | |
| | GAAAGTAAAAAGAGAAAAGTGAAGCATCTCCTGGACTCACCC | 466 |
| | TGAAGTTCTCAGGATCCACGTGCAGCTTGTCACAGTGCAGCT | |
| | CACTCAGCTGAGAAAAAGTGCCCTTGAGGTTGTCCAG | |
| | AGCTGCACGTGGATCCT | 467 |
| | AGGATCCACGTGCAGCT | 468 |
| Thalassaemia Beta | CCCTTTTGCTAATCATGTTCATACCTCTTATCTTCCTCCCACA | 469 |
| LEU-111-PRO | GCTCCTGGGCAACGTGCTGGTCTGTGTGCTGGCCCATCACT | |
| CTG-CCG | TTGGCAAAGAATTCACCCCACCAGTGCAGGCTGCCTA | |
| | TAGGCAGCCTGCACTGGTGGGGTGAATTCTTTGCCAAAGTG | 470 |
| | ATGGGCCAGCACACAGACCAGCACGTTGCCCAGGAGCTGTG | |
| | GGAGGAAGATAAGAGGTATGAACATGATTAGCAAAAGGG | |
| | CAACGTGCTGGTCTGTG | 471 |
| | CACAGACCAGCACGTTG | 472 |
| Thalassaemia Beta | GCTAATCATGTTCATACCTCTTATCTTCCTCCCACAGCTCCTG | 473 |
| CYS-113-Term | GGCAACGTGCTGGTCTGTGTGCTGGCCCATCACTTTGGCAA | |
| TGT to TGA | AGAATTCACCCCACCAGTGCAGGCTGCCTATCAGAAA | |
| | TTTCTGATAGGCAGCCTGCACTGGTGGGGTGAATTCTTTGCC | 474 |
| | AAAGTGATGGGCCAGCACACAGACCAGCACGTTGCCCAGGA | |
| | GCTGTGGGAGGAAGATAAGAGGTATGAACATGATTAGC | |
| | CTGGTCTGTGTGCTGGC | 475 |
| | GCCAGCACACAGACCAG | 476 |
| Thalassaemia Beta | TCATGTTCATACCTCTTATCTTCCTCCCACAGCTCCTGGGCAA | 477 |
| LEU-115-PRO | CGTGCTGGTCTGTGTGCTGGCCCATCACTTTGGCAAAGAATT | |
| CTG to CCG | CACCCCACCAGTGCAGGCTGCCTATCAGAAAGTGGT | |
| | ACCACTTTCTGATAGGCAGCCTGCACTGGTGGGGTGAATTCT | 478 |
| | TTGCCAAAGTGATGGGCCAGCACACAGACCAGCACGTTGCC | |
| | CAGGAGCTGTGGGAGGAAGATAAGAGGTATGAACATGA | |
| | CTGTGTGCTGGCCCATC | 479 |
| | GATGGGCCAGCACACAG | 480 |
| Thalassaemia Beta | TGTTCATACCTCTTATCTTCCTCCCACAGCTCCTGGGCAACG | 481 |
| ALA-116-ASP | TGCTGGTCTGTGTGCTGGCCCATCACTTTGGCAAAGAATTCA | |
| GCC to GAC | CCCCACCAGTGCAGGCTGCCTATCAGAAAGTGGTGGC | |
| | GCCACCACTTTCTGATAGGCAGCCTGCACTGGTGGGGTGAA | 482 |
| | TTCTTTGCCAAAGTGATGGGCCAGCACACAGACCAGCACGTT | |
| | GCCCAGGAGCTGTGGGAGGAAGATAAGAGGTATGAACA | |
| | TGTGCTGGCCCATCACT | 483 |
| | AGTGATGGGCCAGCACA | 484 |
| Thalassaemia Beta | TTCCTCCCACAGCTCCTGGGCAACGTGCTGGTCTGTGTGCT | 485 |
| GLU-122-Term | GGCCCATCACTTTGGCAAAGAATTCACCCCACCAGTGCAGG | |
| GAA to TAA | CTGCCTATCAGAAAGTGGTGGCTGGTGTGGCTAATGCCC | |
| | GGGCATTAGCCACACCAGCCACCACTTTCTGATAGGCAGCC | 486 |
| | TGCACTGGTGGGGTGAATTCTTTGCCAAAGTGATGGGCCAG | |
| | CACACAGACCAGCACGTTGCCCAGGAGCTGTGGGAGGAA | |
| | TTGGCAAAGAATTCACC | 487 |
| | GGTGAATTCTTTGCCAA | 488 |
| Thalassaemia Beta | GCAACGTGCTGGTCTGTGTGCTGGCCCATCACTTTGGCAAA | 489 |
| GLN-128-PRO | GAATTCACCCCACCAGTGCAGGCTGCCTATCAGAAAGTGGT | |
| CAG to CCG | GGCTGGTGTGGCTAATGCCCTGGCCCACAAGTATCACTA | |
| | TAGTGATACTTGTGGGCCAGGGCATTAGCCACACCAGCCAC | 490 |
| | CACTTTCTGATAGGCAGCCTGCACTGGTGGGGTGAATTCTTT | |
| | GCCAAAGTGATGGGCCAGCACACAGACCAGCACGTTGC | |
| | ACCAGTGCAGGCTGCCT | 491 |
| | AGGCAGCCTGCACTGGT | 492 |
| Thalassaemia Beta | GGCAACGTGCTGGTCTGTGTGCTGGCCCATCACTTTGGCAA | 493 |
| GLN-128-Term | AGAATTCACCCCACCAGTGCAGGCTGCCTATCAGAAAGTGGT | |
| CAG to TAG | GGCTGGTGTGGCTAATGCCCTGGCCCACAAGTATCACT | |
| | AGTGATACTTGTGGGCCAGGGCATTAGCCACACCAGCCACC | 494 |
| | ACTTTCTGATAGGCAGCCTGCACTGGTGGGGTGAATTCTTTG | |
| | CCAAAGTGATGGGCCAGCACACAGACCAGCACGTTGCC | |
| | CACCAGTGCAGGCTGCC | 495 |
| | GGCAGCCTGCACTGGTG | 496 |
| Thalassaemia Beta | GTCTGTGTGCTGGCCCATCACTTTGGCAAAGAATTCACCCCA | 497 |
| GLN-132-LYS | CCAGTGCAGGCTGCCTATCAGAAAGTGGTGGCTGGTGTGGC | |
| CAG to AAG | TAATGCCCTGGCCCACAAGTATCACTAAGCTCGCTTTC | |
| | GAAAGCGAGCTTAGTGATACTTGTGGGCCAGGGCATTAGCC | 498 |
| | ACACCAGCCACCACTTTCTGATAGGCAGCCTGCACTGGTGG | |
| | GGTGAATTCTTTGCCAAAGTGATGGGCCAGCACACAGAC | |
| | CTGCCTATCAGAAAGTG | 499 |
| | CACTTTCTGATAGGCAG | 500 |

### EXAMPLE 8

### Retinoblastoma

Retinoblastoma (RB) is an embryonic neoplasm of retinal origin. It almost always presents in early childhood and is often bilateral. The risk of osteogenic sarcoma is increased 500-fold in bilateral retinoblastoma patients, the bone malignancy being at sites removed from those exposed to radiation treatment of the eye tumor.

The retinoblastoma susceptibility gene (pRB;pRb) plays a pivotal role in the regulation of the cell cycle. pRB restrains cell cycle progression by maintaining a checkpoint in late G₁ that controls commitment of cells to enter S phase. The critical role that pRB plays in cell cycle regulation explains its status as archetypal tumor suppressor:loss of pRB function results in an inability to maintain control of the G₁ checkpoint; unchecked progression through the cell cycle is, in turn, a hallmark of neoplasia.

Blanquet et al., Hum. Molec. Genet. 4: 383-388 (1995) performed a mutation survey of the RB1 gene in 232 patients with hereditary or nonhereditary retinoblastoma. They systematically explored all 27 exons and flanking sequences, as well as the promoter. All types of point mutations were represented and found to be unequally distributed along the RB1 gene sequence. In the population studied, exons 3, 8, 18, and 19 were preferentially altered. The attached table discloses the correcting oligonucleotide base sequences for the retinoblastoma oligonucleotides of the invention.

TABLE 9
pRB Mutations and Genome-Correcting Oligos

| Clinical Phenotype & | | SEQ ID |
|---|---|---|
| Mutation | Correcting Oligos | NO: |
| Retinoblastoma | AATATTTGATCTTTATTTTTTGTTCCCAGGGAGGTTATATTCAA | 501 |
| Trp99Term | AAGAAAAAGGAACTGTGGGGAATCTGTATCTTTATTGCAGCA | |
| TGG-TAG | GTTGACCTAGATGAGATGTCGTTCACTTTTACTGA | |
| | TCAGTAAAAGTGAACGACATCTCATCTAGGTCAACTGCTGCA | 502 |
| | ATAAAGATACAGATTCCCCACAGTTCCTTTTTCTTTTGAATATA | |
| | ACCTCCCTGGGAACAAAAAATAAAGATCAAATATT | |
| | GGAACTGTGGGGAATCT | 503 |
| | AGATTCCCCACAGTTCC | 504 |
| Retinoblastoma | ATTTACTTTTTTCTATTCTTTCCTTTGTAGTGTCCATAAATTCTT | 505 |
| Glu137Asp | TAACTTACTAAAAGAAATTGATACCAGTACCAAAGTTGATAAT | |
| GAA-GAT | GCTATGTCAAGACTGTTGAAGAAGTATGATGTA | |
| | TACATCATACTTCTTCAACAGTCTTGACATAGCATTATCAACT | 506 |
| | TTGGTACTGGTATCAATTTCTTTTAGTAAGTTAAAGAATTTATG | |
| | GACACTACAAAGGAAAGAATAGAAAAAAGTAAAT | |
| | CTAAAAGAAATTGATAC | 507 |
| | GTATCAATTTCTTTTAG | 508 |
| Retinoblastoma | TGATTTACTTTTTTCTATTCTTTCCTTTGTAGTGTCCATAAATT | 509 |
| Glu137Term | CTTTAACTTACTAAAAGAAATTGATACCAGTACCAAAGTTGAT | |
| GAA-TAA | AATGCTATGTCAAGACTGTTGAAGAAGTATGATG | |
| | CATCATACTTCTTCAACAGTCTTGACATAGCATTATCAACTTT | 510 |
| | GGTACTGGTATCAATTTCTTTTAGTAAGTTAAAGAATTTATGG | |
| | ACACTACAAAGGAAAGAATAGAAAAAAGTAAATCA | |
| | TACTAAAAGAAATTGAT | 511 |
| | ATCAATTTCTTTTAGTA | 512 |
| Retinoblastoma | AAAATGTTAAAAAGTCATAATGTTTTTCTTTTCAGGACATGTG | 513 |
| Gln176Term | AACTTATATATTTGACACAACCCAGCAGTTCGTAAGTAGTTCA | |
| C | CAGAATGTTATTTTTCACTTAAAAAAAAAGATTTT | |
| | AAAATCTTTTTTTTTAAGTGAAAAATAACATTCTGTGAACTACT | 514 |
| | TACGAACTGCTGGGTTGTGTCAAATATATAAGTTCACATGTCC | |
| | TGAAAAGAAAAACATTATGACTTTTTAACATTTT | |
| | ATTTGACACAACCCAGC | 515 |
| | GCTGGGTTGTGTCAAAT | 516 |
| Retinoblastoma | TGATACATTTTTCCTGTTTTTTTTCTGCTTTCTATTTGTTTAATA | 517 |
| Ile185Thr | GGATATCTACTGAAATAAATTCTGCATTGGTGCTAAAAGTTTC | |
| ATA-ACA | TTGGATCACATTTTTATTAGCTAAAGGTAAGTT | |
| | AACTTACCTTTAGCTAATAAAAATGTGATCCAAGAAACTTTTA | 518 |
| | GCACCAATGCAGAATTTATTTCAGTAGATATCCTATTAAACAA | |
| | ATAGAAAGCAGAAAAAAAACAGGAAAAATGTATCA | |
| | TACTGAAATAAATTCTG | 519 |
| | CAGAATTTATTTCAGTA | 520 |
| Retinoblastoma | AAAGATCTGAATCTCTAACTTTCTTTAAAAATGTACATTTTTTT | 521 |
| Gln207Term | TTCAGGGGAAGTATTACAAATGGAAGATGATCTGGTGATTTC | |
| CAA-TAA | ATTTCAGTTAATGCTATGTGTCCTTGACTATTTTA | |
| | TAAAATAGTCAAGGACACATAGCATTAACTGAAATGAAATCAC | 522 |
| | CAGATCATCTTCCATTTGTAATACTTCCCCTGAAAAAAAAATG | |
| | TACATTTTTAAAGAAAGTTAGAGATTCAGATCTTT | |
| | AAGTATTACAAATGGAA | 523 |
| | TTCCATTTGTAATACTT | 524 |
| Retinoblastoma | GTTCTTATCTAATTTACCACTTTTACAGAAACAGCTGTTATAC | 525 |
| Arg251Term | CCATTAATGGTTCACCTCGAACACCCAGGCGAGGTCAGAACA | |
| CGA to TGA | GGAGTGCACGGATAGCAAAACAACTAGAAAATGATA | |
| | TATCATTTTCTAGTTGTTTTGCTATCCGTGCACTCCTGTTCTG | 526 |
| | ACCTCGCCTGGGTGTTCGAGGTGAACCATTAATGGGTATAAC | |
| | AGCTGTTTCTGTAAAAGTGGTAAATTAGATAAGAAC | |
| | GTTCACCTCGAACACCC | 527 |
| | GGGTGTTCGAGGTGAAC | 528 |
| Retinoblastoma | TTTACCACTTTTACAGAAACAGCTGTTATACCCATTAATGGTT | 529 |
| Arg255Term | CACCTCGAACACCCAGGCGAGGTCAGAACAGGAGTGCACGG | |
| CGA to TGA | ATAGCAAAACAACTAGAAAATGATACAAGAATTATTG | |
| | CAATAATTCTTGTATCATTTTCTAGTTGTTTTGCTATCCGTGCA | 530 |
| | CTCCTGTTCTGACCTCGCCTGGGTGTTCGAGGTGAACCATTA | |
| | ATGGGTATAACAGCTGTTTCTGTAAAAGTGGTAAA | |
| | CACCCAGGCGAGGTCAG | 531 |
| | CTGACCTCGCCTGGGTG | 532 |
| Retinoblastoma | ATTAATGGTTCACCTCGAACACCCAGGCGAGGTCAGAACAG | 533 |
| Gln266Te | GAGTGCACGGATAGCAAAACAACTAGAAAATGATACAAGAAT | |
| | TATTGAAGTTCTCTGTAAAGAACATGAATGTAATATAG | |
| | CTATATTACATTCATGTTCTTTACAGAGAACTTCAATAATTCTT | 534 |
| | GTATCATTTTCTAGTTGTTTTGCTATCCGTGCACTCCTGTTCT | |
| | GACCTCGCCTGGGTGTTCGAGGTGAACCATTAAT | |
| | TAGCAAAACAACTAGAA | 535 |
| | TTCTAGTTGTTTTGCTA | 536 |
| Retinoblastoma | TGACATGTAAAGGATAATTGTCAGTGACTTTTTTCTTTCAAGG | 537 |
| Arg320Term | TTGAAAATCTTTCTAAACGATACGAAGAAATTTATCTTAAAAAT | |
| CGA to TGA | AAAGATCTAGATGCAAGATTATTTTTGGATCATG | |
| | CATGATCCAAAAATAATCTTGCATCTAGATCTTTATTTTTAAGA | 538 |
| | TAAATTTCTTCGTATCGTTTAGAAAGATTTTCAACCTTGAAAG | |
| | AAAAAAGTCACTGACAATTATCCTTTACATGTCA | |
| | TTTCTAAACGATACGAA | 539 |
| | TTCGTATCGTTTAGAAA | 540 |
| Retinoblastoma | ACAAATTGTAAATTTTCAGTATGTGAATGACTTCACTTATTGTT | 541 |
| Gln354Term | ATTTAGTTTTGAAACACAGAGAACACCACGAAAAAGTAACCTT | |
| CAG to TAG | GATGAAGAGGTGAATGTAATTCCTCCACACACTC | |
| | GAGTGTGTGGAGGAATTACATTCACCTCTTCATCAAGGTTAC | 542 |
| | TTTTTCGTGGTGTTCTCTGTGTTTCAAAACTAAATAACAATAA | |
| | GTGAAGTCATTCACATACTGAAAATTTACAATTTGT | |
| | TTGAAACACAGAGAACA | 543 |
| | TGTTCTCTGTGTTTCAA | 544 |
| Retinoblastoma | TTTTCAGTATGTGAATGACTTCACTTATTGTTATTTAGTTTTGA | 545 |
| Arg358Gly | AACACAGAGAACACCACGAAAAAGTAACCTTGATGAAGAGGT | |
| CGA to GGA | GAATGTAATTCCTCCACACACTCCAGTTAGGTATG | |
| | CATACCTAACTGGAGTGTGTGGAGGAATTACATTCACCTCTT | 546 |
| | CATCAAGGTTACTTTTTCGTGGTGTTCTCTGTGTTTCAAAACT | |
| | AAATAACAATAAGTGAAGTCATTCACATACTGAAAA | |
| | GAACACCACGAAAAAGT | 547 |
| | ACTTTTTCGTGGTGTTC | 548 |
| Retinoblastoma | TTTTCAGTATGTGAATGACTTCACTTATTGTTATTTAGTTTTGA | 549 |
| Ar | AACACAGAGAACACCACGAAAAAGTAACCTTGATGAAGAGGT | |
| | GAATGTAATTCCTCCACACACTCCAGTTAGGTATG | |
| | CATACCTAACTGGAGTGTGTGGAGGAATTACATTCACCTCTT | 550 |
| | CATCAAGGTTACTTTTTCGTGGTGTTCTCTGTGTTTCAAAACT | |
| | AAATAACAATAAGTGAAGTCATTCACATACTGAAAA | |
| | GAACACCACGAAAAAGT | 551 |
| | ACTTTTTCGTGGTGTTC | 552 |
| Retinoblastoma | CTGTTATGAACACTATCCAACAATTAATGATGATTTTAAATTCA | 553 |
| Ser397Term | GCAAGTGATCAACCTTCAGAAAATCTGATTTCCTATTTTAACG | |
| TCA to TAA | TAAGCCATATATGAAACATTATTTATTGTAATAT | |
| | ATATTACAATAAATAATGTTTCATATATGGCTTACGTTAAAATA | 554 |
| | GGAAATCAGATTTTCTGAAGGTTGATCACTTGCTGAATTTAAA | |
| | ATCATCATTAATTGTTGGATAGTGTTCATAACAG | |
| | TCAACCTTCAGAAAATC | 555 |
| | GATTTTCTGAAGGTTGA | 556 |
| Retinoblastoma | TTTCATAATTGTGATTTTCTAAAATAGCAGGCTCTTATTTTTCT | 557 |
| Arg445Term | TTTTGTTTGTTTGTAGCGATACAAACTTGGAGTTCGCTTGTAT | |
| CGA to TGA | TACCGAGTAATGGAATCCATGCTTAAATCAGTAA | |
| | TTACTGATTTAAGCATGGATTCCATTACTCGGTAATACAAGCG | 558 |
| | AACTCCAAGTTTGTATCGCTACAAACAAACAAAAAGAAAAATA | |
| | AGAGCCTGCTATTTTAGAAAATCACAATTATGAAA | |
| | GTTTGTAGCGATACAAA | 559 |
| | TTTGTATCGCTACAAAC | 560 |
| Retinoblastoma | GCTCTTATTTTTCTTTTTGTTTGTTTGTAGCGATACAAACTTGG | 561 |
| Arg455Term | AGTTCGCTTGTATTACCGAGTAATGGAATCCATGCTTAAATCA | |
| CGA to TGA | GTAAGTTAAAAACAATATAAAAAAATTTCAGCCG | |
| | CGGCTGAAATTTTTTTATATTGTTTTTAACTTACTGATTTAAGC | 562 |
| | ATGGATTCCATTACTCGGTAATACAAGCGAACTCCAAGTTTGT | |
| | ATCGCTACAAACAAACAAAAAGAAAAATAAGAGC | |
| | TGTATTACCGAGTAATG | 563 |
| | CATTACTCGGTAATACA | 564 |
| Retinoblastoma | ATCGAAAGTTTTATCAAAGCAGAAGGCAACTTGACAAGAGAA | 565 |
| Arg552Term | ATGATAAAACATTTAGAACGATGTGAACATCGAATCATGGAAT | |
| CGA to TGA | CCCTTGCATGGCTCTCAGTAAGTAGCTAAATAATTG | |
| | CAATTATTTAGCTACTTACTGAGAGCCATGCAAGGGATTCCAT | 566 |
| | GATTCGATGTTCACATCGTTCTAAATGTTTTATCATTTCTCTTG | |
| | TCAAGTTGCCTTCTGCTTTGATAAAACTTTCGAT | |
| | ATTTAGAACGATGTGAA | 567 |
| | TTCACATCGTTCTAAAT | 568 |
| Retinoblastoma | AAGTTTTATCAAAGCAGAAGGCAACTTGACAAGAGAAATGAT | 569 |
| Cys553Term | AAAACATTTAGAACGATGTGAACATCGAATCATGGAATCCCTT | |
| TGT to TGA | GCATGGCTCTCAGTAAGTAGCTAAATAATTGAAGAA | |
| | TTCTTCAATTATTTAGCTACTTACTGAGAGCCATGCAAGGGAT | 570 |
| | TCCATGATTCGATGTTCACATCGTTCTAAATGTTTTATCATTTC | |
| | TCTTGTCAAGTTGCCTTCTGCTTTGATAAAACTT | |
| | GAACGATGTGAACATCG | 571 |
| | CGATGTTCACATCGTTC | 572 |
| Retinoblastoma | AGTTTTATCAAAGCAGAAGGCAACTTGACAAGAGAAATGATA | 573 |
| Glu554Term | AAACATTTAGAACGATGTGAACATCGAATCATGGAATCCCTT | |
| GAA to TAA | GCATGGCTCTCAGTAAGTAGCTAAATAATTGAAGAAA | |
| | TTTCTTCAATTATTTAGCTACTTACTGAGAGCCATGCAAGGGA | 574 |
| | TTCCATGATTCGATGTTCACATCGTTCTAAATGTTTTATCATTT | |
| | CTCTTGTCAAGTTGCCTTCTGCTTTGATAAAACT | |
| | AACGATGTGAACATCGA | 575 |
| | TCGATGTTCACATCGTT | 576 |
| Retinoblastoma | TACCTGGGAAAATTATGCTTACTAATGTGGTTTTAATTTCATC | 577 |
| Ser567Leu | ATGTTTCATATAGGATTCACCTTTATTTGATCTTATTAAACAAT | |
| TCA to TTA | CAAAGGACCGAGAAGGACCAACTGATCACCTTGA | |
| | TCAAGGTGATCAGTTGGTCCTTCTCGGTCCTTTGATTGTTTAA | 578 |
| | TAAGATCAAATAAAGGTGAATCCTATATGAAACATGATGAAAT | |
| | TAAAACCACATTAGTAAGCATAATTTTCCCAGGTA | |
| | ATAGGATTCACCTTTAT | 579 |
| | ATAAAGGTGAATCCTAT | 580 |
| Retinoblastoma | AATGTGGTTTTAATTTCATCATGTTTCATATAGGATTCACCTTT | 581 |
| Gln575Term | ATTTGATCTTATTAAACAATCAAAGGACCGAGAAGGACCAACT | |
| CAA to TAA | GATCACCTTGAATCTGCTTGTCCTCTTAATCTTC | |
| | GAAGATTAAGAGGACAAGCAGATTCAAGGTGATCAGTTGGTC | 582 |
| | CTTCTCGGTCCTTTGATTGTTTAATAAGATCAAATAAAGGTGA | |
| | ATCCTATATGAAACATGATGAAATTAAAACCACATT | |
| | TTATTAAACAATCAAAG | 583 |
| | CTTTGATTGTTTAATAA | 584 |
| Retinoblastoma | ATTTCATCATGTTTCATATAGGATTCACCTTTATTTGATCTTAT | 585 |
| Arg579Term | TAAACAATCAAAGGACCGAGAAGGACCAACTGATCACCTTGA | |
| CGA to TGA | ATCTGCTTGTCCTCTTAATCTTCCTCTCCAGAATA | |
| | TATTCTGGAGAGGAAGATTAAGAGGACAAGCAGATTCAAGGT | 586 |
| | GATCAGTTGGTCCTTCTCGGTCCTTTGATTGTTTAATAAGATC | |
| | AAATAAAGGTGAATCCTATATGAAACATGATGAAAT | |
| | CAAAGGACCGAGAAGGA | 587 |
| | TCCTTCTCGGTCCTTTG | 588 |
| Retinoblastoma | TCATCATGTTTCATATAGGATTCACCTTTATTTGATCTTATTA | 589 |
| Glu580Term | ACAATCAAAGGACCGAGAAGGACCAACTGATCACCTTGAATC | |
| GAA to TAA | TGCTTGTCCTCTTAATCTTCCTCTCCAGAATAATC | |
| | GATTATTCTGGAGAGGAAGATTAAGAGGACAAGCAGATTCAA | 590 |
| | GGTGATCAGTTGGTCCTTCTCGGTCCTTTGATTGTTTAATAAG | |
| | ATCAAATAAAGGTGAATCCTATATGAAACATGATGA | |
| | AGGACCGAGAAGGACCA | 591 |
| | TGGTCCTTCTCGGTCCT | 592 |
| Retinoblastoma | AGAAAAAAGGTTCAACTACGCGTGTAAATTCTACTGCAAATG | 593 |
| Ser634Term | CAGAGACACAAGCAACCTCAGCCTTCCAGACCCAGAAGCCA | |
| TCA to TGA | TTGAAATCTACCTCTCTTTCACTGTTTTATAAAAAAGG | |
| | CCTTTTTTATAAAACAGTGAAAGAGAGGTAGATTTCAATGGCT | 594 |
| | TCTGGGTCTGGAAGGCTGAGGTTGCTTGTGTCTCTGCATTTG | |
| | CAGTAGAATTTACACGCGTAGTTGAACCTTTTTTCT | |
| | AGCAACCTCAGCCTTCC | 595 |
| | GGAAGGCTGAGGTTGCT | 596 |
| Retinoblastoma | AAAAAAGGTTCAACTACGCGTGTAAATTCTACTGCAAATGCA | 597 |
| Ala635P | GAGACACAAGCAACCTCAGCCTTCCAGACCCAGAAGCCATT | |
| | GAAATCTACCTCTCTTTCACTGTTTTATAAAAAAGGTT | |
| | AACCTTTTTTATAAAACAGTGAAAGAGAGGTAGATTTCAATGG | 598 |
| | CTTCTGGGTCTGGAAGGCTGAGGTTGCTTGTGTCTCTGCATT | |
| | TGCAGTAGAATTTACACGCGTAGTTGAACCTTTTTT | |
| | CAACCTCAGCCTTCCAG | 599 |
| | CTGGAAGGCTGAGGTTG | 600 |
| Retinoblastoma | ACTACGCGTGTAAATTCTACTGCAAATGCAGAGACACAAGCA | 601 |
| Gln639Term | ACCTCAGCCTTCCAGACCCAGAAGCCATTGAAATCTACCTCT | |
| CAG to TAG | CTTTCACTGTTTTATAAAAAAGGTTAGTAGATGATTA | |
| | TAATCATCTACTAACCTTTTTTATAAAACAGTGAAAGAGAGGT | 602 |
| | AGATTTCAATGGCTTCTGGGTCTGGAAGGCTGAGGTTGCTTG | |
| | TGTCTCTGCATTTGCAGTAGAATTTACACGCGTAGT | |
| | TCCAGACCCAGAAGCCA | 603 |
| | TGGCTTCTGGGTCTGGA | 604 |
| Retinoblastoma | TTGTAATTCAAAATGAACAGTAAAAATGACTAATTTTTCTTATT | 605 |
| L | CCCACAGTGTATCGGCTAGCCTATCTCCGGCTAAATACACTT | |
| | TGTGAACGCCTTCTGTCTGAGCACCCAGAATTAGA | |
| | TCTAATTCTGGGTGCTCAGACAGAAGGCGTTCACAAAGTGTA | 606 |
| | TTTAGCCGGAGATAGGCTAGCCGATACACTGTGGGAATAAG | |
| | AAAAATTAGTCATTTTTACTGTTCATTTTGAATTACAA | |
| | GTATCGGCTAGCCTATC | 607 |
| | GATAGGCTAGCCGATAC | 608 |
| Retinoblastoma | AATGAACAGTAAAAATGACTAATTTTTCTTATTCCCACAGTGT | 609 |
| Arg661Trp | ATCGGCTAGCCTATCTCCGGCTAAATACACTTTGTGAACGCC | |
| CGG to TGG | TTCTGTCTGAGCACCCAGAATTAGAACATATCATCT | |
| | AGATGATATGTTCTAATTCTGGGTGCTCAGACAGAAGGCGTT | 610 |
| | CACAAAGTGTATTTAGCCGGAGATAGGCTAGCCGATACACTG | |
| | TGGGAATAAGAAAAATTAGTCATTTTTACTGTTCATT | |
| | CCTATCTCCGGCTAAAT | 611 |
| | ATTTAGCCGGAGATAGG | 612 |
| Retinoblastoma | AACAGTAAAAATGACTAATTTTTCTTATTCCCACAGTGTATCG | 613 |
| Leu662Pro | GCTAGCCTATCTCCGGCTAAATACACTTTGTGAACGCCTTCT | |
| CTA to CCA | GTCTGAGCACCCAGAATTAGAACATATCATCTGGAC | |
| | GTCCAGATGATATGTTCTAATTCTGGGTGCTCAGACAGAAGG | 614 |
| | CGTTCACAAAGTGTATTTAGCCGGAGATAGGCTAGCCGATAC | |
| | ACTGTGGGAATAAGAAAAATTAGTCATTTTTACTGTT | |
| | TCTCCGGCTAAATACAC | 615 |
| | GTGTATTTAGCCGGAGA | 616 |
| Retinoblastoma | TATCGGCTAGCCTATCTCCGGCTAAATACACTTTGTGAACGC | 617 |
| Glu675Term | CTTCTGTCTGAGCACCCAGAATTAGAACATATCATCTGGACC | |
| GAA to TAA | CTTTTCCAGCACACCCTGCAGAATGAGTATGAACTCA | |
| | TGAGTTCATACTCATTCTGCAGGGTGTGCTGGAAAAGGGTCC | 618 |
| | AGATGATATGTTCTAATTCTGGGTGCTCAGACAGAAGGCGTT | |
| | CACAAAGTGTATTTAGCCGGAGATAGGCTAGCCGATA | |
| | AGCACCCAGAATTAGAA | 619 |
| | TTCTAATTCTGGGTGCT | 620 |
| Retinoblastoma | TTTGTGAACGCCTTCTGTCTGAGCACCCAGAATTAGAACATA | 621 |
| Gln685Pro | TCATCTGGACCCTTTTCCAGCACACCCTGCAGAATGAGTATG | |
| CAG to CCG | AACTCATGAGAGACAGGCATTTGGACCAAGTAAGAAA | |
| | TTTCTTACTTGGTCCAAATGCCTGTCTCTCATGAGTTCATACT | 622 |
| | CATTCTGCAGGGTGTGCTGGAAAAGGGTCCAGATGATATGTT | |
| | CTAATTCTGGGTGCTCAGACAGAAGGCGTTCACAAA | |
| | CCTTTTCCAGCACACCC | 623 |
| | GGGTGTGCTGGAAAAGG | 624 |
| Retinoblastoma | AAAACCATGTAATAAAATTCTGACTACTTTTACATCAATTTATT | 625 |
| Cys706Tyr | TACTAGATTATGATGTGTTCCATGTATGGCATATGCAAAGTGA | |
| TGT to TAT | AGAATATAGACCTTAAATTCAAAATCATTGTAAC | |
| | GTTACAATGATTTTGAATTTAAGGTCTATATTCTTCACTTTGCA | 626 |
| | TATGCCATACATGGAACACATCATAATCTAGTAAATAAATTGA | |
| | TGTAAAAGTAGTCAGAATTTTATTACATGGTTTT | |
| | TATGATGTGTTCCATGT | 627 |
| | ACATGGAACACATCATA | 628 |
| Retinoblastoma | TTCTGACTACTTTTACATCAATTTATTTACTAGATTATGATGTG | 629 |
| Cys712Arg | TTCCATGTATGGCATATGCAAAGTGAAGAATATAGACCTTAAA | |
| TGC to CGC | TTCAAAATCATTGTAACAGCATACAAGGATCTTC | |
| | GAAGATCCTTGTATGCTGTTACAATGATTTTGAATTTAAGGTC | 630 |
| | TATATTCTTCACTTTGCATATGCCATACATGGAACACATCATA | |
| | ATCTAGTAAATAAATTGATGTAAAAGTAGTCAGAA | |
| | ATGGCATATGCAAAGTG | 631 |
| | CACTTTGCATATGCCAT | 632 |
| Retinoblastom | GTATGGCATATGCAAAGTGAAGAATATAGACCTTAAATTCAAA | 633 |
| TAC to TAA | ATCATTGTAACAGCATACAAGGATCTTCCTCATGCTGTTCAG | |
| | GAGGTAGGTAATTTTCCATAGTAAGTTTTTTTGATA | |
| | TATCAAAAAAACTTACTATGGAAAATTACCTACCTCCTGAACA | 634 |
| | GCATGAGGAAGATCCTTGTATGCTGTTACAATGATTTTGAATT | |
| | TAAGGTCTATATTCTTCACTTTGCATATGCCATAC | |
| | ACAGCATACAAGGATCT | 635 |
| | AGATCCTTGTATGCTGT | 636 |
| Retinoblastoma | TTTTTTTTTTTTTTTACTGTTCTTCCTCAGACATTCAAACGTGT | 637 |
| Glu748Term | TTTGATCAAAGAAGAGGAGTATGATTCTATTATAGTATTCTAT | |
| GAG to TAG | AACTCGGTCTTCATGCAGAGACTGAAAACAAATA | |
| | TATTTGTTTTCAGTCTCTGCATGAAGACCGAGTTATAGAATAC | 638 |
| | TATAATAGAATCATACTCCTCTTCTTTGATCAAAACACGTTTG | |
| | AATGTCTGAGGAAGAACAGTAAAAAAAAAAAAAAA | |
| | AAGAAGAGGAGTATGAT | 639 |
| | ATCATACTCCTCTTCTT | 640 |
| Retinoblastoma | GTTTTGATCAAAGAAGAGGAGTATGATTCTATTATAGTATTCT | 641 |
| Gln762Term | ATAACTCGGTCTTCATGCAGAGACTGAAAACAAATATTTTGCA | |
| CAG to TAG | GTATGCTTCCACCAGGGTAGGTCAAAAGTATCCTT | |
| | AAGGATACTTTTGACCTACCCTGGTGGAAGCATACTGCAAAA | 642 |
| | TATTTGTTTTCAGTCTCTGCATGAAGACCGAGTTATAGAATAC | |
| | TATAATAGAATCATACTCCTCTTCTTTGATCAAAAC | |
| | TCTTCATGCAGAGACTG | 643 |
| | CAGTCTCTGCATGAAGA | 644 |
| Retinoblastoma | TAATCTACTTTTTTGTTTTTGCTCTAGCCCCCTACCTTGTCAC | 645 |
| Arg787Term | CAATACCTCACATTCCTCGAAGCCCTTACAAGTTTCCTAGTTC | |
| CGA-TGA | ACCCTTACGGATTCCTGGAGGGAACATCTATATTT | |
| | AAATATAGATGTTCCCTCCAGGAATCCGTAAGGGTGAACTAG | 646 |
| | GAAACTTGTAAGGGCTTCGAGGAATGTGAGGTATTGGTGACA | |
| | AGGTAGGGGGCTAGAGCAAAAACAAAAAAGTAGATTA | |
| | ACATTCCTCGAAGCCCT | 647 |
| | AGGGCTTCGAGGAATGT | 648 |
| Retinoblastoma | CCTTACGGATTCCTGGAGGGAACATCTATATTTCACCCCTGA | 649 |
| Ser816Term | AGAGTCCATATAAAATTTCAGAAGGTCTGCCAACACCAACAA | |
| TCA to TGA | AAATGACTCCAAGATCAAGGTGTGTGTTTTCTCTTTA | |
| | TAAAGAGAAAACACACACCTTGATCTTGGAGTCATTTTTGTTG | 650 |
| | GTGTTGGCAGACCTTCTGAAATTTTATATGGACTCTTCAGGG | |
| | GTGAAATATAGATGTTCCCTCCAGGAATCCGTAAGG | |
| | TAAAATTTCAGAAGGTC | 651 |
| | GACCTTCTGAAATTTTA | 652 |

### EXAMPLE 9

### BRCA1 and BRCA2

Breast cancer is the second major cause of cancer death in American women, with an estimated 44,190 lives lost (290 men and 43,900 women) in the US in 1997. While ovarian cancer accounts for fewer deaths than breast cancer, it still represents 4% of all female cancers. In 1994, two breast cancer susceptibility genes were identified: BRCA1 on chromosome 17 and BRCA2 on chromosome 13. When a woman carries a mutation in either BRCA1 or BRCA2, she is at increased risk of being diagnosed with breast or ovarian cancer at some point in her life.

Ford et al., Am. J. Hum. Genet. 62: 676-689 (1998) assessed the contribution of BRCA1 and BRCA2 to inherited breast cancer by linkage and mutation analysis in 237 families, each with at least 4 cases of breast cancer. Families were included without regard to the occurrence of ovarian or other cancers. Overall, disease was linked to BRCA1 in an estimated 52% of families, to BRCA2 in 32% of families, and to neither gene in 16%, suggesting other predisposition genes. The majority (81 %) of the breast-ovarian cancer families were due to BRCA1, with most others (14%) due to BRCA2. Conversely, the majority (76%) of families with both male and female breast cancer were due to BRCA2. The largest proportion (67%) of families due to other genes were families with 4 or 5 cases of female breast cancer only.

More than 75% of the reported mutations in the BRCA1 gene result in truncated proteins. Couch et al., Hum. Mutat. 8: 8-18, 1996. (1996) reported a total of 254 BRCA1 mutations, 132 (52%) of which were unique. A total of 221 (87%) of all mutations or 107 (81 %) of the unique mutations are small deletions, insertions, nonsense point mutations, splice variants, and regulatory mutations that result in truncation or absence of the BRCA1 protein. A total of 11 disease-associated missense mutations (5 unique) and 21 variants (19 unique) as yet unclassified as missense mutations or polymorphisms had been detected. Thirty-five independent benign polymorphisms had been described. The most common mutations were 185delAG and 5382insC, which accounted for 30 (11.7%) and 26 (10.1%), respectively, of all the mutations.

Most BRCA2 mutations are predicted to result in a truncated protein product. The smallest known cancer-associated deletion removes from the C terminus only 224 of the 3,418 residues constituting BRCA2, suggesting that these terminal amino acids are critical for BRCA2 function. Studies (Spain et al., Proc. Natl. Acad. Sci. 96:13920-13925 (1999)) suggest that such truncations eliminate or interfere with 2 nuclear localization signals that reside within the final 156 residues of BRCA2, suggesting that the vast majority of BRCA2 mutants are nonfunctional because they are not translocated into the nucleus.

The attached table discloses the correcting oligonucleotide base sequences for the BRACA1 and BRACA2 oligonucleotides of the invention.

TABLE 10
BRCA1 Mutations and Genome-Correcting Oligos

| | | |
|---|---|---|
| Clinical Phenotype & | | SEQ ID |
| Mutation | Correcting Oligos | NO: |

| | | |
|---|---|---|
| Breast Cancer | CTGCGCTCAGGAGGCCTTCACCCTCTGCTCTGGGTAAAGTT | 653 |
| Met-1-Ile | CATTGGAACAGAAAGAAATGGATTTATCTGCTCTTCGCGTTG | |
| ATG to ATT | AAGAAGTACAAAATGTCATTAATGCTATGCAGAAAATC | |
| | GATTTTCTGCATAGCATTAATGACATTTTGTACTTCTTCAACG | 654 |
| | CGAAGAGCAGATAAATCCATTTCTTTCTGTTCCAATGAACTTT | |
| | ACCCAGAGCAGAGGGTGAAGGCCTCCTGAGCGCAG | |
| | AAAGAAATGGATTTATC | 655 |
| | GATAAATCCATTTCTTT | 656 |
| Breast Cancer | CTGGGTAAAGTTCATTGGAACAGAAAGAAATGGATTTATCTG | 657 |
| Val-11-Ala | CTCTTCGCGTTGAAGAAGTACAAAATGTCATTAATGCTATGCA | |
| GTA to GCA | GAAAATCTTAGAGTGTCCCATCTGTCTGGAGTTGAT | |
| | ATCAACTCCAGACAGATGGGACACTCTAAGATTTTCTGCATA | 658 |
| | GCATTAATGACATTTTGTACTTCTTCAACGCGAAGAGCAGATA | |
| | AATCCATTTCTTTCTGTTCCAATGAACTTTACCCAG | |
| | TGAAGAAGTACAAAATG | 659 |
| | CATTTTGTACTTCTTCA | 660 |
| Breast Cancer | ATGGATTTATCTGCTCTTCGCGTTGAAGAAGTACAAAATGTCA | 661 |
| Ile-21-Val | TTAATGCTATGCAGAAAATCTTAGAGTGTCCCATCTGTCTGG | |
| ATC to GTC | AGTTGATCAAGGAACCTGTCTCCACAAAGTGTGACC | |
| | GGTCACACTTTGTGGAGACAGGTTCCTTGATCAACTCCAGAC | 662 |
| | AGATGGGACACTCTAAGATTTTCTGCATAGCATTAATGACATT | |
| | TTGTACTTCTTCAACGCGAAGAGCAGATAAATCCAT | |
| | TGCAGAAAATCTTAGAG | 663 |
| | CTCTAAGATTTTCTGCA | 664 |
| Breast Cancer | ATTTATCTGCTCTTCGCGTTGAAGAAGTACAAAATGTCATTAA | 665 |
| Leu-22-Ser | TGCTATGCAGAAAATCTTAGAGTGTCCCATCTGTCTGGAGTT | |
| TTA to TCA | GATCAAGGAACCTGTCTCCACAAAGTGTGACCACAT | |
| | ATGTGGTCACACTTTGTGGAGACAGGTTCCTTGATCAACTCC | 666 |
| | AGACAGATGGGACACTCTAAGATTTTCTGCATAGCATTAATG | |
| | ACATTTTGTACTTCTTCAACGCGAAGAGCAGATAAAT | |
| | GAAAATCTTAGAGTGTC | 667 |
| | GACACTCTAAGATTTTC | 668 |
| Breast Cancer | AGAAAATCTTAGAGTGTCCCATCTGTCTGGAGTTGATCAAGG | 669 |
| Cys-39-Tyr | AACCTGTCTCCACAAAGTGTGACCACATATTTTGCAAATTTTG | |
| TGT to TAT | CATGCTGAAACTTCTCAACCAGAAGAAAGGGCCTTC | |
| | GAAGGCCCTTTCTTCTGGTTGAGAAGTTTCAGCATGCAAAAT | 670 |
| | TTGCAAAATATGTGGTCACACTTTGTGGAGACAGGTTCCTTG | |
| | ATCAACTCCAGACAGATGGGACACTCTAAGATTTTCT | |
| | CACAAAGTGTGACCACA | 671 |
| | TGTGGTCACACTTTGTG | 672 |
| Breast Cancer | CACATATTTTGCAAATTTTGCATGCTGAAACTTCTCAACCAGA | 673 |
| Cys-61-Gly | AGAAAGGGCCTTCACAGTGTCCTTTATGTAAGAATGATATAA | |
| TGT to GGT | CCAAAAGGAGCCTACAAGAAAGTACGAGATTTAGTC | |
| | GACTAAATCTCGTACTTTCTTGTAGGCTCCTTTTGGTTATATC | 674 |
| | ATTCTTACATAAAGGACACTGTGAAGGCCCTTTCTTCTGGTT | |
| | GAGAAGTTTCAGCATGCAAAATTtGCAAAATATGTG | |
| | CTTCACAGTGTCCTTTA | 675 |
| | TAAAGGACACTGTGAAG | 676 |
| Breast Cancer | TTTGCAAATTTTGCATGCTGAAACTTCTCAACCAGAAGAAAGG | 677 |
| Leu-63-Stop | GCCTTCACAGTGTCCTTTATGTAAGAATGATATAACCAAAAGG | |
| TTA to TAA | AGCCTACAAGAAAGTACGAGATTTAGTCAACTTGT | |
| | ACAAGTTGACTAAATCTCGTACTTTCTTGTAGGCTCCTTTTGG | 678 |
| | TTATATCATTCTTACATAAAGGACACTGTGAAGGCCCTTTCTT | |
| | CTGGTTGAGAAGTTTCAGCATGCAAAATTTGCAAA | |
| | GTGTCCTTTATGTAAGA | 679 |
| | TCTTACATAAAGGACAC | 680 |
| Breast Cancer | TGCAAATTTTGCATGCTGAAACTTCTCAACCAGAAGAAAGGG | 681 |
| Cys-64-Arg | CCTTCACAGTGTCCTTTATGTAAGAATGATATAACCAAAAGGA | |
| TGT to CGT | GCCTACAAGAAAGTACGAGATTTAGTCAACTTGTTG | |
| Breast Cancer | CAACAAGTTGACTAAATCTCGTACTTTCTTGTAGGCTCCTTTT | 682 |
| Cys-64-Gly | GGTTATATCATTCTTACATAAAGGACACTGTGAAGGCCCTTTC | |
| TGT to GGT | TTCTGGTTGAGAAGTTTCAGCATGCAAAATTTGCA | |
| | GTCCTTTATGTAAGAAT | 683 |
| | ATTCTTACATAAAGGAC | 684 |
| Breast Cancer | GCAAATTTTGCATGCTGAAACTTCTCAACCAGAAGAAAGGGC | 685 |
| Cys-64-Tyr | CTTCACAGTGTCCTTTATGTAAGAATGATATAACCAAAAGGAG | |
| TGT to TAT | CCTACAAGAAAGTACGAGATTTAGTCAACTTGTTGA | |
| | TCAACAAGTTGACTAAATCTCGTACTTTCTTGTAGGCTCCTTT | 686 |
| | TGGTTATATCATTCTTACATAAAGGACACTGTGAAGGCCCTTT | |
| | CTTCTGGTTGAGAAGTTTCAGCATGCAAAATTTGC | |
| | TCCTTTATGTAAGAATG | 687 |
| | CATTCTTACATAAAGGA | 688 |
| Breast Cancer | CAGAAGAAAGGGCCTTCACAGTGTCCTTTATGTAAGAATGAT | 689 |
| Gln-74-Stop | ATAACCAAAAGGAGCCTACAAGAAAGTACGAGATTTAGTCAA | |
| CAA to TAA | CTTGTTGAAGAGCTATTGAAAATCATTTGTGCTTTTC | |
| | GAAAAGCACAAATGATTTTCAATAGCTCTTCAACAAGTTGACT | 690 |
| | AAATCTCGTACTTTCTTGTAGGCTCCTTTTGGTTATATCATTCT | |
| | TACATAAAGGACACTGTGAAGGCCCTTTCTTCTG | |
| | GGAGCCTACAAGAAAGT | 691 |
| | ACTTTCTTGTAGGCTCC | 692 |
| Breast Cancer | AGCTATTGAAAATCATTTGTGCTTTTCAGCTTGACACAGGTTT | 693 |
| Tyr-105-Cys | GGAGTATGCAAACAGCTATAATTTTGCAAAAAAGGAAAATAA | |
| TAT to TGT | CTCTCCTGAACATCTAAAAGATGAAGTTTCTATCAT | |
| | ATGATAGAAACTTCATCTTTTAGATGTTCAGGAGAGTTATTTT | 694 |
| | CCTTTTTTGCAAAATTATAGCTGTTTGCATACTCCAAACCTGT | |
| | GTCAAGCTGAAAAGCACAAATGATTTTCAATAGCT | |
| | AAACAGCTATAATTTTG | 695 |
| | CAAAATTATAGCTGTTT | 696 |
| Breast Cancer | CTACAGAGTGAACCCGAAAATCCTTCCTTGCAGGAAACCAGT | 697 |
| Asn-158-Tyr | CTCAGTGTCCAACTCTCTAACCTTGGAACTGTGAGAACTCTG | |
| AAC to TAC | AGGACAAAGCAGCGGATACAACCTCAAAAGACGTCTG | |
| | CAGACGTCTTTTGAGGTTGTATCCGCTGCTTTGTCCTCAGAG | 698 |
| | TTCTCACAGTTCCAAGGTTAGAGAGTTGGACACTGAGACTGG | |
| | TTTCCTGCAAGGAAGGATTTTCGGGTTCACTCTGTAG | |
| | AACTCTCTAACCTTGGA | 699 |
| | TCCAAGGTTAGAGAGTT | 700 |
| Breast Cancer | GAAACCAGTCTCAGTGTCCAACTCTCTAACCTTGGAACTGTG | 701 |
| Gln-169-Stop | AGAACTCTGAGGACAAAGCAGCGGATACAACCTCAAAAGAC | |
| CAG to TAG | GTCTGTCTACATTGAATTGGGATCTGATTCTTCTGAAG | |
| | CTTCAGAAGAATCAGATCCCAATTCAATGTAGACAGACGTCT | 702 |
| | TTTGAGGTTGTATCCGCTGCTTTGTCCTCAGAGTTCTCACAG | |
| | TTCCAAGGTTAGAGAGTTGGACACTGAGACTGGTTTC | |
| | GGACAAAGCAGCGGATA | 703 |
| | TATCCGCTGCTTTGTCC | 704 |
| Breast Cancer | CTCCCAGCACAGAAAAAAAGGTAGATCTGAATGCTGATCCCC | 705 |
| Trp-353-Stop | TGTGTGAGAGAAAAGAATGGAATAAGCAGAAACTGCCATGCT | |
| TGG to TAG | CAGAGAATCCTAGAGATACTGAAGATGTTCCTTGGAT | |
| | ATCCAAGGAACATCTTCAGTATCTCTAGGATTCTCTGAGCAT | 706 |
| | GGCAGTTTCTGCTTATTCCATTCTTTTCTCTCACACAGGGGAT | |
| | CAGCATTCAGATCTACCTTTTTTTCTGTGCTGGGAG | |
| | AAAAGAATGGAATAAGC | 707 |
| | GCTTATTCCATTCTTTT | 708 |
| Breast Cancer | ATGCTCAGAGAATCCTAGAGATACTGAAGATGTTCCTTGGAT | 709 |
| Ile-379-Met | AACACTAAATAGCAGCATTCAGAAAGTTAATGAGTGGTTTTCC | |
| ATT to ATG | AGAAGTGATGAACTGTTAGGTTCTGATGACTCACAT | |
| | ATGTGAGTCATCAGAACCTAACAGTTCATCACTTCTGGAAAA | 710 |
| | CCACTCATTAACTTTCTGAATGCTGCTATTTAGTGTTATCCAA | |
| | GGAACATCTTCAGTATCTCTAGGATTCTCTGAGCAT | |
| | AGCAGCATTCAGAAAGT | 711 |
| | ACTTTCTGAATGCTGCT | 712 |
| Breast Cancer | GGGAGTCTGAATCAAATGCCAAAGTAGCTGATGTATTGGACG | 713 |
| Glu-421-Gly | TTCTAAATGAGGTAGATGAATATTCTGGTTCTTCAGAGAAAAT | |
| GAA to GGA | AGACTTACTGGCCAGTGATCCTCATGAGGCTTTAAT | |
| | ATTAAAGCCTCATGAGGATCACTGGCCAGTAAGTCTATTTTCT | 714 |
| | CTGAAGAACCAGAATATTCATCTACCTCATTTAGAACGTCCAA | |
| | TACATCAGCTACTTTGGCATTTGATTCAGACTCCC | |
| | GGTAGATGAATATTCTG | 715 |
| | CAGAATATTCATCTACC | 716 |
| Breast Cancer | ATATGTAAAAGTGAAAGAGTTCACTCCAAATCAGTAGAGAGT | 717 |
| Phe-461-Leu | AATATTGAAGACAAAATATTTGGGAAAACCTATCGGAAGAAG | |
| TTT to CTT | GCAAGCCTCCCCAACTTAAGCCATGTAACTGAAAATC | |
| | GATTTTCAGTTACATGGCTTAAGTTGGGGAGGCTTGCCTTCT | 718 |
| | TCCGATAGGTTTTCCCAAATATTTTGTCTTCAATATTACTCTCT | |
| | ACTGATTTGGAGTGAACTCTTTCACTTTTACATAT | |
| | ACAAAATATTTGGGAAA | 719 |
| | TTTCCCAAATATTTTGT | 720 |
| Breast Cancer | GAAAGAGTTCACTCCAAATCAGTAGAGAGTAATATTGAAGAC | 721 |
| Tyr-465-Leu | AAAATATTTGGGAAAACCTATCGGAAGAAGGCAAGCCTCCCC | |
| TAT to GAT | AACTTAAGCCATGTAACTGAAAATCTAATTATAGGAG | |
| | CTCCTATAATTAGATTTTCAGTTACATGGCTTAAGTTGGGGAG | 722 |
| | GCTTGCCTTCTTCCGATAGGTTTTCCCAAATATTTTGTCTTCA | |
| | ATATTACTCTCTACTGATTTGGAGTGAACTCTTTC | |
| | GGAAAACCTATCGGAAG | 723 |
| | CTTCCGATAGGTTTTCC | 724 |
| Breast Cancer | ACCTATCGGAAGAAGGCAAGCCTCCCCAACTTAAGCCATGTA | 725 |
| Gly-484-Stop | ACTGAAAATCTAATTATAGGAGCATTTGTTACTGAGCCACAGA | |
| GGA to TGA | TAATACAAGAGCGTCCCCTCACAAATAAATTAAAGC | |
| | GCTTTAATTTATTTGTGAGGGGACGCTCTTGTATTATCTGTGG | 726 |
| | CTCAGTAACAAATGCTCCTATAATTAGATTTTCAGTTACATGG | |
| | CTTAAGTTGGGGAGGCTTGCCTTCTTCCGATAGGT | |
| | TAATTATAGGAGCATTT | 727 |
| | AAATGCTCCTATAATTA | 728 |
| Breast Cancer | TTACTGAGCCACAGATAATACAAGAGCGTCCCCTCACAAATA | 729 |
| Arg-507-Ile | AATTAAAGCGTAAAAGGAGACCTACATCAGGCCTTCATCCTG | |
| AGA to ATA | AGGATTTTATCAAGAAAGCAGATTTGGCAGTTCAAAA | |
| | TTTTGAACTGCCAAATCTGCTTTCTTGATAAAATCCTCAGGAT | 730 |
| | GAAGGCCTGATGTAGGTCTCCTTTTACGCTTTAATTTATTTGT | |
| | GAGGGGACGCTCTTGTATTATCTGTGGCTCAGTAA | |
| | TAAAAGGAGACCTACAT | 731 |
| | ATGTAGGTCTCCTTTTA | 732 |
| Breast Cancer | CACAGATAATACAAGAGCGTCCCCTCACAAATAAATTAAAGC | 733 |
| Ser-510-Stop | GTAAAAGGAGACCTACATCAGGCCTTCATCCTGAGGATTTTA | |
| TCA to TGA | TCAAGAAAGCAGATTTGGCAGTTCAAAAGACTCCTGA | |
| | TCAGGAGTCTTTTGAACTGCCAAATCTGCTTTCTTGATAAAAT | 734 |
| | CCTCAGGATGAAGGCCTGATGTAGGTCTCCTTTTACGCTTTA | |
| | ATTTATTTGTGAGGGGACGCTCTTGTATTATCTGTG | |
| | ACCTACATCAGGCCTTC | 735 |
| | GAAGGCCTGATGTAGGT | 736 |
| Breast Cancer | AGGAGACCTACATCAGGCCTTCATCCTGAGGATTTTATCAAG | 737 |
| Gln-526-Stop | AAAGCAGATTTGGCAGTTCAAAAGACTCCTGAAATGATAAAT | |
| CAA to TAA | CAGGGAACTAACCAAACGGAGCAGAATGGTCAAGTGA | |
| | TCACTTGACCATTCTGCTCCGTTTGGTTAGTTCCCTGATTTAT | 738 |
| | CATTTCAGGAGTCTTTTGAACTGCCAAATCTGCTTTCTTGATA | |
| | AAATCCTCAGGATGAAGGCCTGATGTAGGTCTCCT | |
| | TGGCAGTTCAAAAGACT | 739 |
| | AGTCTTTTGAACTGCCA | 740 |
| Breast Cancer | AGGAGACCTACATCAGGCCTTCATCCTGAGGATTTTATCAAG | 741 |
| Gln-541-Stop | AAAGCAGATTTGGCAGTTCAAAAGACTCCTGAAATGATAAAT | |
| CAG to TAG | CAGGGAACTAACCAAACGGAGCAGAATGGTCAAGTGA | |
| | TCACTTGACCATTCTGCTCCGTTTGGTTAGTTCCCTGATTTAT | 742 |
| | CATTTCAGGAGTCTTTTGAACTGCCAAATCTGCTTTCTTGATA | |
| | AAATCCTCAGGATGAAGGCCTGATGTAGGTCTCCT | |
| | AAACGGAGCAGAATGGT | 743 |
| | ACCATTCTGCTCCGTTT | 744 |
| Breast Cancer | TAAATCAGGGAACTAACCAAACGGAGCAGAATGGTCAAGTGA | 745 |
| Gly-552-Val | TGAATATTACTAATAGTGGTCATGAGAATAAAACAAAAGGTGA | |
| GGT to GTT | TTCTATTCAGAATGAGAAAAATCCTAACCCAATAGA | |
| | TCTATTGGGTTAGGATTTTTCTCATTCTGAATAGAATCACCTT | 746 |
| | TTGTTTTATTCTCATGACCACTATTAGTAATATTCATCACTTGA | |
| | CCATTCTGCTCCGTTTGGTTAGTTCCCTGATTTA | |
| | TAATAGTGGTCATGAGA | 747 |
| | TCTCATGACCACTATTA | 748 |
| Breast Cancer | GGTCAAGTGATGAATATTACTAATAGTGGTCATGAGAATAAAA | 749 |
| Gln-563-Stop | CAAAAGGTGATTCTATTCAGAATGAGAAAAATCCTAACCCAAT | |
| CAG to TAG | AGAATCACTCGAAAAAGAATCTGCTTTCAAAACGA | |
| | TCGTTTTGAAAGCAGATTCTTTTTCGAGTGATTCTATTGGGTT | 750 |
| | AGGATTTTTCTCATTCTGAATAGAATCACCTTTTGTTTTATTCT | |
| | CATGACCACTATTAGTAATATTCATCACTTGACC | |
| | ATTCTATTCAGAATGAG | 751 |
| | CTCATTCTGAATAGAAT | 752 |
| Ovarian Cancer | ATAAGCAGCAGTATAAGCAATATGGAACTCGAATTAAATATCC | 753 |
| Lys-607-Stop | ACAATTCAAAAGCACCTAAAAAGAATAGGCTGAGGAGGAAGT | |
| AAA to TAA | CTTCTACCAGGCATATTCATGCGCTTGAACTAGTAG | |
| | CTACTAGTTCAAGCGCATGAATATGCCTGGTAGAAGACTTCC | 754 |
| | TCCTCAGCCTATTCTTTTTAGGTGCTTTTGAATTGTGGATATT | |
| | TAATTCGAGTTCCATATTGCTTATACTGCTGCTTAT | |
| | AAGCACCTAAAAAGAAT | 755 |
| | ATTCTTTTTAGGTGCTT | 756 |
| Breast Cancer | ATATTCATGCGCTTGAACTAGTAGTCAGTAGAAATCTAAGCC | 757 |
| Leu-639-Stop | CACCTAATTGTACTGAATTGCAAATTGATAGTTGTTCTAGCAG | |
| TTG to TAG | TGAAGAGATAAAGAAAAAAAAGTACAACCAAATGCC | |
| | GGCATTTGGTTGTACTTTTTTTTCTTTATCTCTTCACTGCTAGA | 758 |
| | ACAACTATCAATTTGCAATTCAGTACAATTAGGTGGGCTTAGA | |
| | TTTCTACTGACTACTAGTTCAAGCGCATGAATAT | |
| | TACTGAATTGCAAATTG | 759 |
| | CAATTTGCAATTCAGTA | 760 |
| Breast Cancer | GAACCTGCAACTGGAGCCAAGAAGAGTAACAAGCCAAATGA | 761 |
| Asp-693-Asn | ACAGACAAGTAAAAGACATGACAGCGATACTTTCCCAGAGCT | |
| GAC to AAC | GAAGTTAACAAATGCACCTGGTTCTTTTACTAAGTGTT | |
| | AACACTTAGTAAAAGAACCAGGTGCATTTGTTAACTTCAGCTC | 762 |
| | TGGGAAAGTATCGCTGTCATGTCTTTTACTTGTCTGTTCATTT | |
| | GGCTTGTTACTCTTCTTGGCTCCAGTTGCAGGTTC | |
| | AAAGACATGACAGCGAT | 763 |
| | ATCGCTGTCATGTCTTT | 764 |
| Ovarian Cancer | CTGAAGTTAACAAATGCACCTGGTTCTTTTACTAAGTGTTCAA | 765 |
| Glu-720-Stop | ATACCAGTGAACTTAAAGAATTTGTCAATCCTAGCCTTCCAAG | |
| GAA to TAA | AGAAGAAAAAGAAGAGAAACTAGAAACAGTTAAAG | |
| | CTTTAACTGTTTCTAGTTTCTCTTCTTTTTCTTCTCTTGGAAGG | 766 |
| | CTAGGATTGACAAATTCTTTAAGTTCACTGGTATTTGAACACT | |
| | TAGTAAAAGAACCAGGTGCATTTGTTAACTTCAG | |
| | AACTTAAAGAATTTGTC | 767 |
| | GACAAATTCTTTAAGTT | 768 |
| Breast Cancer | CTAGAAACAGTTAAAGTGTCTAATAATGCTGAAGACCCCAAA | 769 |
| Glu-755-Stop | GATCTCATGTTAAGTGGAGAAAGGGTTTTGCAAACTGAAAGA | |
| GAA to TAA | TCTGTAGAGAGTAGCAGTATTTCATTGGTACCTGGTA | |
| | TACCAGGTACCAATGAAATACTGCTACTCTCTACAGATCTTTC | 770 |
| | AGTTTGCAAAACCCTTTCTCCACTTAACATGAGATCTTTGGGG | |
| | TCTTCAGCATTATTAGACACTTTAACTGTTTCTAG | |
| | TAAGTGGAGAAAGGGTT | 771 |
| | AACCCTTTCTCCACTTA | 772 |
| Breast Cancer | TCATGTTAAGTGGAGAAAGGGTTTTGCAAACTGAAAGATCTG | 773 |
| Ser-770-Stop | TAGAGAGTAGCAGTATTTCATTGGTACCTGGTACTGATTATG | |
| TCA to TAA | GCACTCAGGAAAGTATCTCGTTACTGGAAGTTAGCAC | |
| | GTGCTAACTTCCAGTAACGAGATACTTTCCTGAGTGCCATAA | 774 |
| | TCAGTACCAGGTACCAATGAAATACTGCTACTCTCTACAGAT | |
| | CTTTCAGTTTGCAAAACCCTTTCTCCACTTAACATGA | |
| | CAGTATTTCATTGGTAC | 775 |
| | GTACCAATGAAATACTG | 776 |
| Breast Cancer | TAAGTGGAGAAAGGGTTTTGCAAACTGAAAGATCTGTAGAGA | 777 |
| Val-772-Ala | GTAGCAGTATTTCATTGGTACCTGGTACTGATTATGGCACTC | |
| GTA to GCA | AGGAAAGTATCTCGTTACTGGAAGTTAGCACTCTAGG | |
| | CCTAGAGTGCTAACTTCCAGTAACGAGATACTTTCCTGAGTG | 778 |
| | CCATAATCAGTACCAGGTACCAATGAAATACTGCTACTCTCTA | |
| | CAGATCTTTCAGTTTGCAAAACCCTTTCTCCACTTA | |
| | TTCATTGGTACCTGGTA | 779 |
| | TACCAGGTACCAATGAA | 780 |
| Breast Cancer | ACTGAAAGATCTGTAGAGAGTAGCAGTATTTCATTGGTACCT | 781 |
| Gln-780-Stop | GGTACTGATTATGGCACTCAGGAAAGTATCTCGTTACTGGAA | |
| CAG to TAG | GTTAGCACTCTAGGGAAGGCAAAAACAGAACCAAATA | |
| | TATTTGGTTCTGTTTTTGCCTTCCCTAGAGTGCTAACTTCCAG | 782 |
| | TAACGAGATACTTTCCTGAGTGCCATAATCAGTACCAGGTAC | |
| | CAATGAAATACTGCTACTCTCTACAGATCTTTCAGT | |
| | ATGGCACTCAGGAAAGT | 783 |
| | ACTTTCCTGAGTGCCAT | 784 |
| Breast Cancer | TATGGCACTCAGGAAAGTATCTCGTTACTGGAAGTTAGCACT | 785 |
| Glu-797-Stop | CTAGGGAAGGCAAAAACAGAACCAAATAAATGTGTGAGTCAG | |
| GAA to TAA | TGTGCAGCATTTGAAAACCCCAAGGGACTAATTCATG | |
| | CATGAATTAGTCCCTTGGGGTTTTCAAATGCTGCACACTGAC | 786 |
| | TCACACATTTATTTGGTTCTGTTTTTGCCTTCCCTAGAGTGCT | |
| | AACTTCCAGTAACGAGATACTTTCCTGAGTGCCATA | |
| | CAAAAACAGAACCAAAT | 787 |
| | ATTTGGTTCTGTTTTTG | 788 |
| Breast Cancer | AAATGTGTGAGTCAGTGTGCAGCATTTGAAAACCCCAAGGGA | 789 |
| Lys-820-Glu | CTAATTCATGGTTGTTCCAAAGATAATAGAAATGACACAGAAG | |
| AAA to GAA | GCTTTAAGTATCCATTGGGACATGAAGTTAACCACA | |
| | TGTGGTTAACTTCATGTCCCAATGGATACTTAAAGCCTTCTGT | 790 |
| | GTCATTTCTATTATCTTTGGAACAACCATGAATTAGTCCCTTG | |
| | GGGTTTTCAAATGCTGCACACTGACTCACACATTT | |
| | GTTGTTCCAAAGATAAT | 791 |
| | ATTATCTTTGGAACAAC | 792 |
| Breast Cancer | CAGCATTTGAAAACCCCAAGGGACTAATTCATGGTTGTTCCA | 793 |
| Thr-826-Lys | AAGATAATAGAAATGACACAGAAGGCTTTAAGTATCCATTGG | |
| ACA to AAA | GACATGAAGTTAACCACAGTCGGGAAACAAGCATAGA | |
| | TCTATGCTTGTTTCCCGACTGTGGTTAACTTCATGTCCCAATG | 794 |
| | GATACTTAAAGCCTTCTGTGTCATTTCTATTATCTTTGGAACA | |
| | ACCATGAATTAGTCCCTTGGGGTTTTCAAATGCTG | |
| | AAATGACACAGAAGGCT | 795 |
| | AGCCTTCTGTGTCATTT | 796 |
| Breast Cancer | GATAATAGAAATGACACAGAAGGCTTTAAGTATCCATTGGGA | 797 |
| Arg-841-Trp | CATGAAGTTAACCACAGTCGGGAAACAAGCATAGAAATGGAA | |
| CGG to TGG | GAAAGTGAACTTGATGCTCAGTATTTGCAGAATACAT | |
| | ATGTATTCTGCAAATACTGAGCATCAAGTTCACTTTCTTCCAT | 798 |
| | TTCTATGCTTGTTTCCCGACTGTGGTTAACTTCATGTCCCAAT | |
| | GGATACTTAAAGCCTTCTGTGTCATTTCTATTATC | |
| | ACCACAGTCGGGAAACA | 799 |
| | TGTTTCCCGACTGTGGT | 800 |
| Breast Cancer | AACTTGATGCTCAGTATTTGCAGAATACATTCAAGGTTTCAAA | 801 |
| Pro-871-Leu | GCGCCAGTCATTTGCTCCGTTTTCAAATCCAGGAAATGCAGA | |
| CCG to CTG | AGAGGAATGTGCAACATTCTCTGCCCACTCTGGGTC | |
| | GACCCAGAGTGGGCAGAGAATGTTGCACATTCCTCTTCTGCA | 802 |
| | TTTCCTGGATTTGAAAACGGAGCAAATGACTGGCGCTTTGAA | |
| | ACCTTGAATGTATTCTGCAAATACTGAGCATCAAGTT | |
| | ATTTGCTCCGTTTTCAA | 803 |
| | TTGAAAACGGAGCAAAT | 804 |
| Breast Cancer | TTTCAAATCCAGGAAATGCAGAAGAGGAATGTGCAACATTCT | 805 |
| Leu-892-Ser | CTGCCCACTCTGGGTCCTTAAAGAAACAAAGTCCAAAAGTCA | |
| TTA to TCA | CTTTTGAATGTGAACAAAAGGAAGAAAATCAAGGAAA | |
| | TTTCCTTGATTTTCTTCCTTTTGTTCACATTCAAAAGTGACTTT | 806 |
| | TGGACTTTGTTTCTTTAAGGACCCAGAGTGGGCAGAGAATGT | |
| | TGCACATTCCTCTTCTGCATTTCCTGGATTTGAAA | |
| | TGGGTCCTTAAAGAAAC | 807 |
| | GTTTCTTTAAGGACCCA | 808 |
| Breast Cancer | CACTCTGGGTCCTTAAAGAAACAAAGTCCAAAAGTCACTTTT | 809 |
| Glu-908-Stop | GAATGTGAACAAAAGGAAGAAAATCAAGGAAAGAATGAGTCT | |
| GAA to TAA | AATATCAAGCCTGTACAGACAGTTAATATCACTGCAG | |
| | CTGCAGTGATATTAACTGTCTGTACAGGCTTGATATTAGACTC | 810 |
| | ATTCTTTCCTTGATTTTCTTCCTTTTGTTCACATTCAAAAGTGA | |
| | CTTTTGGACTTTGTTTCTTTAAGGACCCAGAGTG | |
| | AAAAGGAAGAAAATCAA | 811 |
| | TTGATTTTCTTCCTTTT | 812 |
| Breast Cancer | ATAATGCCAAATGTAGTATCAAAGGAGGCTCTAGGTTTTGTCT | 813 |
| Gly-960-Asp | ATCATCTCAGTTCAGAGGCAACGAAACTGGACTCATTACTCC | |
| GGC to GAC | AAATAAACATGGACTTTTACAAAACCCATATCGTAT | |
| | ATACGATATGGGTTTTGTAAAAGTCCATGTTTATTTGGAGTAA | 814 |
| | TGAGTCCAGTTTCGTTGCCTCTGAACTGAGATGATAGACAAA | |
| | ACCTAGAGCCTCCTTTGATACTACATTTGGCATTAT | |
| | GTTCAGAGGCAACGAAA | 815 |
| | TTTCGTTGCCTCTGAAC | 816 |
| Breast Cancer | ATTTGTTAAAACTAAATGTAAGAAAAATCTGCTAGAGGAAAAC | 817 |
| Met-1008-Ile | TTTGAGGAACATTCAATGTCACCTGAAAGAGAAATGGGAAAT | |
| ATG to ATA | GAGAACATTCCAAGTACAGTGAGCACAATTAGCCGT | |
| | ACGGCTAATTGTGCTCACTGTACTTGGAATGTTCTCATTTCCC | 818 |
| | ATTTCTCTTTCAGGTGACATTGAATGTTCCTCAAAGTTTTCCT | |
| | CTAGCAGATTTTTCTTACATTTAGTTTTAACAAAT | |
| | CATTCAATGTCACCTGA | 819 |
| | TCAGGTGACATTGAATG | 820 |
| Breast Cancer | ACTTTGAGGAACATTCAATGTCACCTGAAAGAGAAATGGGAA | 821 |
| Thr-1025-Ile | ATGAGAACATTCCAAGTACAGTGAGCACAATTAGCCGTAATA | |
| ACA to ATA | ACATTAGAGAAAATGTTTTTAAAGAAGCCAGCTCAAG | |
| | CTTGAGCTGGCTTCTTTAAAAACATTTTCTCTAATGTTATTAC | 822 |
| | GGCTAATTGTGCTCACTGTACTTGGAATGTTCTCATTTCCCAT | |
| | TTCTCTTTCAGGTGACATTGAATGTTCCTCAAAGT | |
| | TCCAAGTACAGTGAGCA | 823 |
| | TGCTCACTGTACTTGGA | 824 |
| Breast Cancer | ACATTCCAAGTACAGTGAGCACAATTAGCCGTAATAACATTA | 825 |
| Glu-1038-Gly | GAGAAAATGTTTTTAAAGAAGCCAGCTCAAGCAATATTAATGA | |
| GAA to GGA | AGTAGGTTCCAGTACTAATGAAGTGGGCTCCAGTAT | |
| | ATACTGGAGCCCACTTCATTAGTACTGGAACCTACTTCATTAA | 826 |
| | TATTGCTTGAGCTGGCTTCTTTAAAAACATTTTCTCTAATGTTA | |
| | TTACGGCTAATTGTGCTCACTGTACTTGGAATGT | |
| | TTTTAAAGAAGCCAGCT | 827 |
| | AGCTGGCTTCTTTAAAA | 828 |
| Breast Cancer | CAAGTACAGTGAGCACAATTAGCCGTAATAACATTAGAGAAA | 829 |
| Ser-1040-Asn | ATGTTTTTAAAGAAGCCAGCTCAAGCAATATTAATGAAGTAGG | |
| AGC to AAC | TTCCAGTACTAATGAAGTGGGCTCCAGTATTAATGA | |
| | TCATTAATACTGGAGCCCACTTCATTAGTACTGGAACCTACTT | 830 |
| | CATTAATATTGCTTGAGCTGGCTTCTTTAAAAACATTTTCTCTA | |
| | ATGTTATTACGGCTAATTGTGCTCACTGTACTTG | |
| | AGAAGCCAGCTCAAGCA | 831 |
| | TGCTTGAGCTGGCTTCT | 832 |
| Breast Cancer | GCCGTAATAACATTAGAGAAAATGTTTTTAAAGAAGCCAGCTC | 833 |
| Val-1047-Ala | AAGCAATATTAATGAAGTAGGTTCCAGTACTAATGAAGTGGG | |
| GTA to GCA | CTCCAGTATTAATGAAATAGGTTCCAGTGATGAAAA | |
| | TTTTCATCACTGGAACCTATTTCATTAATACTGGAGCCCACTT | 834 |
| | CATTAGTACTGGAACCTACTTCATTAATATTGCTTGAGCTGGC | |
| | TTCTTTAAAAACATTTTCTCTAATGTTATTACGGC | |
| | TAATGAAGTAGGTTCCA | 835 |
| | TGGAACCTACTTCATTA | 836 |
| Breast Cancer | AAATAGGTTCCAGTGATGAAAACATTCAAGCAGAACTAGGTA | 837 |
| Leu-1080-Stop | GAAACAGAGGGCCAAAATTGAATGCTATGCTTAGATTAGGGG | |
| TTG to TAG | TTTTGCAACCTGAGGTCTATAAACAAAGTCTTCCTGG | |
| | CCAGGAAGACTTTGTTTATAGACCTCAGGTTGCAAAACCCCT | 838 |
| | AATCTAAGCATAGCATTCAATTTTGGCCCTCTGTTTCTACCTA | |
| | GTTCTGCTTGAATGTTTTCATCACTGGAACCTATTT | |
| | GCCAAAATTGAATGCTA | 839 |
| | TAGCATTCAATTTTGGC | 840 |
| Breast Cancer | AAAACATTCAAGCAGAACTAGGTAGAAACAGAGGGCCAAAAT | 841 |
| Leu-1086-Stop | TGAATGCTATGCTTAGATTAGGGGTTTTGCAACCTGAGGTCT | |
| TTA to TGA | ATAAACAAAGTCTTCCTGGAAGTAATTGTAAGCATCC | |
| | GGATGCTTACAATTACTTCCAGGAAGACTTTGTTTATAGACCT | 842 |
| | CAGGTTGCAAAACCCCTAATCTAAGCATAGCATTCAATTTTG | |
| | GCCCTCTGTTTCTACCTAGTTCTGCTTGAATGTTTT | |
| | GCTTAGATTAGGGGTTT | 843 |
| | AAACCCCTAATCTAAGC | 844 |
| Breast Cancer | AGCAAGAATATGAAGAAGTAGTTCAGACTGTTAATACAGATTT | 845 |
| Ser-1130-Stop | CTCTCCATATCTGATTTCAGATAACTTAGAACAGCCTATGGGA | |
| TCA to TGA | AGTAGTCATGCATCTCAGGTTTGTTCTGAGACACC | |
| | GGTGTCTCAGAACAAACCTGAGATGCATGACTACTTCCCATA | 846 |
| | GGCTGTTCTAAGTTATCTGAAATCAGATATGGAGAGAAATCT | |
| | GTATTAACAGTCTGAACTACTTCTTCATATTCTTGCT | |
| | TCTGATTTCAGATAACT | 847 |
| | AGTTATCTGAAATCAGA | 848 |
| Breast Cancer | CTAGTTTTGCTGAAAATGACATTAAGGAAAGTTCTGCTGTTTT | 849 |
| Lys-1183-Arg | TAGCAAAAGCGTCCAGAAAGGAGAGCTTAGCAGGAGTCCTA | |
| AAA to AGA | GCCCTTTCACCCATACACATTTGGCTCAGGGTTACCG | |
| | CGGTAACCCTGAGCCAAATGTGTATGGGTGAAAGGGCTAGG | 850 |
| | ACTCCTGCTAAGCTCTCCTTTCTGGACGCTTTTGCTAAAAACA | |
| | GCAGAACTTTCCTTAATGTCATTTTCAGCAAAACTAG | |
| | CGTCCAGAAAGGAGAGC | 851 |
| | GCTCTCCTTTCTGGACG | 852 |
| Breast Cancer | AGCGTCCAGAAAGGAGAGCTTAGCAGGAGTCCTAGCCCTTT | 853 |
| Gln-1200-Stop | CACCCATACACATTTGGCTCAGGGTTACCGAAGAGGGGCCA | |
| CAG to TAG | AGAAATTAGAGTCCTCAGAAGAGAACTTATCTAGTGAGG | |
| | CCTCACTAGATAAGTTCTCTTCTGAGGACTCTAATTTCTTGGC | 854 |
| | CCCTCTTCGGTAACCCTGAGCCAAATGTGTATGGGTGAAAGG | |
| | GCTAGGACTCCTGCTAAGCTCTCCTTTCTGGACGCT | |
| | ATTTGGCTCAGGGTTAC | 855 |
| | GTAACCCTGAGCCAAAT | 856 |
| Breast Cancer | AAAGGAGAGCTTAGCAGGAGTCCTAGCCCTTTCACCCATACA | 857 |
| Arg-1203-Stop | CATTTGGCTCAGGGTTACCGAAGAGGGGCCAAGAAATTAGA | |
| CGA to TGA | GTCCTCAGAAGAGAACTTATCTAGTGAGGATGAAGAGC | |
| | GCTCTTCATCCTCACTAGATAAGTTCTCTTCTGAGGACTCTAA | 858 |
| | TTTCTTGGCCCCTCTTCGGTAACCCTGAGCCAAATGTGTATG | |
| | GGTGAAAGGGCTAGGACTCCTGCTAAGCTCTCCTTT | |
| | AGGGTTACCGAAGAGGG | 859 |
| | CCCTCTTCGGTAACCCT | 860 |
| Breast Cancer | ACCCATACACATTTGGCTCAGGGTTACCGAAGAGGGGCCAA | 861 |
| Glu-1214-Stop | GAAATTAGAGTCCTCAGAAGAGAACTTATCTAGTGAGGATGA | |
| GAG to TAG | AGAGCTTCCCTGCTTCCAACACTTGTTATTTGGTAAAG | |
| | CTTTACCAAATAACAAGTGTTGGAAGCAGGGAAGCTCTTCAT | 862 |
| | CCTCACTAGATAAGTTCTCTTCTGAGGACTCTAATTTCTTGGC | |
| | CCCTCTTCGGTAACCCTGAGCCAAATGTGTATGGGT | |
| | CCTCAGAAGAGAACTTA | 863 |
| | TAAGTTCTCTTCTGAGG | 864 |
| Breast Cancer | TCAGGGTTACCGAAGAGGGGCCAAGAAATTAGAGTCCTCAG | 865 |
| Glu-1219-Asp | AAGAGAACTTATCTAGTGAGGATGAAGAGCTTCCCTGCTTCC | |
| GAG to GAC | AACACTTGTTATTTGGTAAAGTAAACAATATACCTTCT | |
| | AGAAGGTATATTGTTTACTTTACCAAATAACAAGTGTTGGAAG | 866 |
| | CAGGGAAGCTCTTCATCCTCACTAGATAAGTTCTCTTCTGAG | |
| | GACTCTAATTTCTTGGCCCCTCTTCGGTAACCCTGA | |
| | TCTAGTGAGGATGAAGA | 867 |
| | TCTTCATCCTCACTAGA | 868 |
| Breast Cancer | GGTTACCGAAGAGGGGCCAAGAAATTAGAGTCCTCAGAAGA | 869 |
| Glu-1221-Stop | GAACTTATCTAGTGAGGATGAAGAGCTTCCCTGCTTCCAACA | |
| GAA to TAA | CTTGTTATTTGGTAAAGTAAACAATATACCTTCTCAGT | |
| | ACTGAGAAGGTATATTGTTTACTTTACCAAATAACAAGTGTTG | 870 |
| | GAAGCAGGGAAGCTCTTCATCCTCACTAGATAAGTTCTCTTC | |
| | TGAGGACTCTAATTTCTTGGCCCCTCTTCGGTAACC | |
| | GTGAGGATGAAGAGCTT | 871 |
| | AAGCTCTTCATCCTCAC | 872 |
| Breast Cancer | TTATTTGGTAAAGTAAACAATATACCTTCTCAGTCTACTAGGC | 873 |
| Glu-1250-Stop | ATAGCACCGTTGCTACCGAGTGTCTGTCTAAGAACACAGAGG | |
| GAG to TAG | AGAATTTATTATCATTGAAGAATAGCTTAAATGACT | |
| | AGTCATTTAAGCTATTCTTCAATGATAATAAATTCTCCTCTGTG | 874 |
| | TTCTTAGACAGACACTCGGTAGCAACGGTGCTATGCCTAGTA | |
| | GACTGAGAAGGTATATTGTTTACTTTACCAAATAA | |
| | TTGCTACCGAGTGTCTG | 875 |
| | CAGACACTCGGTAGCAA | 876 |
| Breast Cancer | CTAGGCATAGCACCGTTGCTACCGAGTGTCTGTCTAAGAACA | 877 |
| Ser-1262-Stop | CAGAGGAGAATTTATTATCATTGAAGAATAGCTTAAATGACTG | |
| TCA to TAA | CAGTAACCAGGTAATATTGGCAAAGGCATCTCAGGA | |
| | TCCTGAGATGCCTTTGCCAATATTACCTGGTTACTGCAGTCAT | 878 |
| | TTAAGCTATTCTTCAATGATAATAAATTCTCCTCTGTGTTCTTA | |
| | GACAGACACTCGGTAGCAACGGTGCTATGCCTAG | |
| | TTTATTATCATTGAAGA | 879 |
| | TCTTCAATGATAATAAA | 880 |
| Breast Cancer | TTATCATTGAAGAATAGCTTAAATGACTGCAGTAACCAGGTAA | 881 |
| Gln-1281-Stop | TATTGGCAAAGGCATCTCAGGAACATCACCTTAGTGAGGAAA | |
| CAG to TAG | CAAAATGTTCTGCTAGCTTGTTTTCTTCACAGTGCA | |
| | TGCACTGTGAAGAAAACAAGCTAGCAGAACATTTTGTTTCCT | 882 |
| | CACTAAGGTGATGTTCCTGAGATGCCTTTGCCAATATTACCT | |
| | GGTTACTGCAGTCATTTAAGCTATTCTTCAATGATAA | |
| | AGGCATCTCAGGAACAT | 883 |
| | ATGTTCCTGAGATGCCT | 884 |
| Breast Cancer | GCTAGCTTGTTTTCTTCACAGTGCAGTGAATTGGAAGACTTG | 885 |
| Gln-1313-Stop | ACTGCAAATACAAACACCCAGGATCCTTTCTTGATTGGTTCTT | |
| CAG to TAG | CCAAACAAATGAGGCATCAGTCTGAAAGCCAGGGAG | |
| | CTCCCTGGCTTTCAGACTGATGCCTCATTTGTTTGGAAGAAC | 886 |
| | CAATCAAGAAAGGATCCTGGGTGTTTGTATTTGCAGTCAAGT | |
| | CTTCCAATTCACTGCACTGTGAAGAAAACAAGCTAGC | |
| | CAAACACCCAGGATCCT | 887 |
| | AGGATCCTGGGTGTTTG | 888 |
| Breast Cancer | TCACAGTGCAGTGAATTGGAAGACTTGACTGCAAATACAAAC | 889 |
| Ile-1318-Val | ACCCAGGATCCTTTCTTGATTGGTTCTTCCAAACAAATGAGG | |
| ATT to GTT | CATCAGTCTGAAAGCCAGGGAGTTGGTCTGAGTGACA | |
| | TGTCACTCAGACCAACTCCCTGGCTTTCAGACTGATGCCTCA | 890 |
| | TTTGTTTGGAAGAACCAATCAAGAAAGGATCCTGGGTGTTTG | |
| | TATTTGCAGTCAAGTCTTCCAATTCACTGCACTGTGA | |
| | CTTTCTTGATTGGTTCT | 891 |
| | AGAACCAATCAAGAAAG | 892 |
| Breast Cancer | TTGGAAGACTTGACTGCAAATACAAACACCCAGGATCCTTTC | 893 |
| Gln-1323-Stop | TTGATTGGTTCTTCCAAACAAATGAGGCATCAGTCTGAAAGC | |
| CAA to TAA | CAGGGAGTTGGTCTGAGTGACAAGGAATTGGTTTCAG | |
| | CTGAAACCAATTCCTTGTCACTCAGACCAACTCCCTGGCTTT | 894 |
| | CAGACTGATGCCTCATTTGTTTGGAAGAACCAATCAAGAAAG | |
| | GATCCTGGGTGTTTGTATTTGCAGTCAAGTCTTCCAA | |
| | CTTCCAAACAAATGAGG | 895 |
| | CCTCATTTGTTTGGAAG | 896 |
| Breast Cancer | CAGTCTGAAAGCCAGGGAGTTGGTCTGAGTGACAAGGAATT | 897 |
| Arg-1347-Gly | GGTTTCAGATGATGAAGAAAGAGGAACGGGCTTGGAAGAAA | |
| AGA to GGA | ATAATCAAGAAGAGCAAAGCATGGATTCAAACTTAGGTA | |
| | TACCTAAGTTTGAATCCATGCTTTGCTCTTCTTGATTATTTTCT | 898 |
| | TCCAAGCCCGTTCCTCTTTCTTCATCATCTGAAACCAATTCCT | |
| | TGTCACTCAGACCAACTCCCTGGCTTTCAGACTG | |
| | ATGAAGAAAGAGGAACG | 899 |
| | CGTTCCTCTTTCTTCAT | 900 |
| Breast Cancer | GAAACAAGCGTCTCTGAAGACTGCTCAGGGCTATCCTCTCAG | 901 |
| Gln-1395-Stop | AGTGACATTTTAACCACTCAGGTAAAAAGCGTGTGTGTGTGT | |
| CAG to TAG | GCACATGCGTGTGTGTGGTGTCCTTTGCATTCAGTAG | |
| | CTACTGAATGCAAAGGACACCACACACACGCATGTGCACACA | 902 |
| | CACACACGCTTTTTACCTGAGTGGTTAAAATGTCACTCTGAG | |
| | AGGATAGCCCTGAGCAGTCTTCAGAGACGCTTGTTTC | |
| | TAACCACTCAGGTAAAA | 903 |
| | TTTTACCTGAGTGGTTA | 904 |
| Breast Cancer | TGGTGCCATTTATCGTTTTTGAAGCAGAGGGATACCATGCAA | 905 |
| Gln-1408-Stop | CATAACCTGATAAAGCTCCAGCAGGAAATGGCTGAACTAGAA | |
| CAG to TAG | GCTGTGTTAGAACAGCATGGGAGCCAGCCTTCTAACA | |
| | TGTTAGAAGGCTGGCTCCCATGCTGTTCTAACACAGCTTCTA | 906 |
| | GTTCAGCCATTTCCTGCTGGAGCTTTATCAGGTTATGTTGCAT | |
| | GGTATCCCTCTGCTTCAAAAACGATAAATGGCACCA | |
| | TAAAGCTCCAGCAGGAA | 907 |
| | TTCCTGCTGGAGCTTTA | 908 |
| Breast Cancer | AGCCAGCCTTCTAACAGCTACCCTTCCATCATAAGTGACTCT | 909 |
| Arg-1443-Gly | TCTGCCCTTGAGGACCTGCGAAATCCAGAACAAAGCACATCA | |
| CGA to GGA | GAAAAAGGTGTGTATTGTTGGCCAAACACTGATATCT | |
| Arg-1443-Stop | AGATATCAGTGTTTGGCCAACAATACACACCTTTTTCTGATGT | 910 |
| CGA to TGA | GCTTTGTTCTGGATTTCGCAGGTCCTCAAGGGCAGAAGAGTC | |
| | ACTTATGATGGAAGGGTAGCTGTTAGAAGGCTGGCT | |
| | AGGACCTGCGAAATCCA | 911 |
| | TGGATTTCGCAGGTCCT | 912 |
| Breast Cancer | CAGAATAGAAACTACCCATCTCAAGAGGAGCTCATTAAGGTT | 913 |
| Ser-1512-Ile | GTTGATGTGGAGGAGCAACAGCTGGAAGAGTCTGGGCCACA | |
| AGT to ATT | CGATTTGACGGAAACATCTTACTTGCCAAGGCAAGATC | |
| | GATCTTGCCTTGGCAAGTAAGATGTTTCCGTCAAATCGTGTG | 914 |
| | GCCCAGACTCTTCCAGCTGTTGCTCCTCCACATCAACAACCT | |
| | TAATGAGCTCCTCTTGAGATGGGTAGTTTCTATTCTG | |
| | AGGAGCAACAGGTGGAA | 915 |
| | TTCCAGCTGTTGCTCCT | 916 |
| Breast Cancer | ATCTTTCTAGGTCATCCCCTTCTAAATGCCCATCATTAGATGA | 917 |
| Gln-1538-Stop | TAGGTGGTACATGCACAGTTGCTCTGGGAGTCTTCAGAATAG | |
| CAG to TAG | AAACTACCCATCTCAAGAGGAGCTCATTAAGGTTGT | |
| | ACAACCTTAATGAGCTCCTCTTGAGATGGGTAGTTTCTATTCT | 918 |
| | GAAGACTCCCAGAGCAACTGTGCATGTACCACCTATCATCTA | |
| | ATGATGGGCATTTAGAAGGGGATGACCTAGAAAGAT | |
| | CATGCACAGTTGCTCTG | 919 |
| | CAGAGCAACTGTGCATG | 920 |
| Breast Cancer | CAGAATAGAAACTACCCATCTCAAGAGGAGCTCATTAAGGTT | 921 |
| Glu-1541-Stop | GTTGATGTGGAGGAGCAACAGCTGGAAGAGTCTGGGCCACA | |
| GAG to TAG | CGATTTGACGGAAACATCTTACTTGCCAAGGCAAGATG | |
| | GATCTTGCCTTGGCAAGTAAGATGTTTCCGTCAAATCGTGTG | 922 |
| | GCCCAGAGTCTTCCAGCTGTTGCTCCTCCACATCAACAACCT | |
| | TAATGAGCTCCTCTTGAGATGGGTAGTTTCTATTCTG | |
| | AGGAGCAACAGCTGGAA | 923 |
| | TTCCAGCTGTTGCTCCT | 924 |
| Breast Cancer | AACTACCCATCTCAAGAGGAGCTCATTAAGGTTGTTGATGTG | 925 |
| Thr-1561-Ile | GAGGAGCAACAGCTGGAAGAGTCTGGGCCACACGATTTGAC | |
| ACG to ATC | GGAAACATCTTACTTGCCAAGGCAAGATCTAGGTAATA | |
| | TATTACCTAGATCTTGCCTTGGCAAGTAAGATGTTTCCGTCAA | 926 |
| | ATCGTGTGGCCCAGAGTCTTCCAGCTGTTGCTCCTCCACATC | |
| | AACAACCTTAATGAGCTCCTCTTGAGATGGGTAGTT | |
| | AGCTGGAAGAGTCTGGG | 927 |
| | CCCAGACTCTTCCAGCT | 928 |
| Breast Cancer | TTTGTAATTCAACATTCATCGTTGTGTAAATTAAACTTCTCCCA | 929 |
| Tyr-1563-Stop | TTCCTTTCAGAGGGAACCCCTTACCTGGAATCTGGAATCAGC | |
| TAC to TAG | CTCTTCTCTGATGACCCTGAATCTGATCCTTCTGA | |
| | TCAGAAGGATCAGATTCAGGGTCATCAGAGAAGAGGCTGATT | 930 |
| | CCAGATTCCAGGTAAGGGGTTCCCTCTGAAAGGAATGGGAG | |
| | AAGTTTAATTTACACAACGATGAATGTTGAATTACAAA | |
| | AGAGGGAACCCCTTACC | 931 |
| | GGTAAGGGGTTCCCTCT | 932 |
| Breast Cancer | CAACATTCATCGTTGTGTAAATTAAACTTCTCCCATTCCTTTC | 933 |
| Leu-1564-Pro | AGAGGGAACCCCTTACCTGGAATCTGGAATCAGCCTCTTCTC | |
| CTG to CCG | TGATGACCCTGAATCTGATCCTTCTGAAGACAGAGC | |
| | GCTCTGTCTTCAGAAGGATCAGATTCAGGGTCATCAGAGAAG | 934 |
| | AGGCTGATTCCAGATTCCAGGTAAGGGGTTCCCTCTGAAAG | |
| | GAATGGGAGAAGTTTAATTTACACAACGATGAATGTTG | |
| | CCCTTACCTGGAATCTG | 935 |
| | CAGATTCCAGGTAAGGG | 936 |
| Breast Cancer | GCCCCAGAGTCAGCTCGTGTTGGCAACATACCATCTTCAACC | 937 |
| Gln-1604-Stop | TCTGCATTGAAAGTTCCCCAATTGAAAGTTGCAGAATCTGCC | |
| CAA to TAA | CAGAGTCCAGCTGCTGCTCATACTACTGATACTGCTG | |
| | CAGCAGTATCAGTAGTATGAGCAGCAGCTGGACTCTGGGCA | 938 |
| | GATTCTGCAACTTTCAATTGGGGAACTTTCAATGCAGAGGTT | |
| | GAAGATGGTATGTTGCCAACACGAGCTGACTCTGGGGC | |
| | AAGTTCCCCAATTGAAA | 939 |
| | TTTCAATTGGGGAACTT | 940 |
| Breast Cancer | GAGTCAGCTCGTGTTGGCAACATACCATCTTCAACCTCTGCA | 941 |
| Lys-1606-Glu | TTGAAAGTTCCCCAATTGAAAGTTGCAGAATCTGCCCAGAGT | |
| AAA to GAA | CCAGCTGCTGCTCATACTACTGATACTGCTGGGTATA | |
| | TATACCCAGCAGTATCAGTAGTATGAGCAGCAGCTGGACTCT | 942 |
| | GGGCAGATTCTGCAACTTTCAATTGGGGAACTTTCAATGCAG | |
| | AGGTTGAAGATGGTATGTTGCCAACACGAGCTGACTC | |
| | CCCAATTGAAAGTTGCA | 943 |
| | TGCAACTTTCAATTGGG | 944 |
| Breast Cancer | CAGAATCTGCCCAGAGTCCAGCTGCTGCTCATACTACTGATA | 945 |
| Met-1628-Thr | CTGCTGGGTATAATGCAATGGAAGAAAGTGTGAGCAGGGAG | |
| ATG to ACG | AAGCCAGAATTGACAGCTTCAACAGAAAGGGTCAACAA | |
| | TTGTTGACCCTTTCTGTTGAAGCTGTCAATTCTGGCTTCTCCC | 946 |
| | TGCTCACACTTTCTTCCATTGCATTATACCCAGCAGTATCAGT | |
| | AGTATGAGCAGCAGCTGGACTCTGGGCAGATTCTG | |
| | TAATGCAATGGAAGAAA | 947 |
| | TTTCTTCCATTGCATTA | 948 |
| Breast Cancer | GCAGAATCTGCCCAGAGTCCAGCTGCTGCTCATACTACTGAT | 949 |
| Met-1628-Val | ACTGCTGGGTATAATGCAATGGAAGAAAGTGTGAGCAGGGA | |
| ATG to GTG | GAAGCCAGAATTGACAGCTTCAACAGAAAGGGTCAACA | |
| | TGTTGACCCTTTCTGTTGAAGCTGTCAATTCTGGCTTCTCCCT | 950 |
| | GCTCACACTTTCTTCCATTGCATTATACCCAGCAGTATCAGTA | |
| | GTATGAGCAGCAGCTGGACTCTGGGCAGATTCTGC | |
| | ATAATGCAATGGAAGAA | 951 |
| | TTCTTCCATTGCATTAT | 952 |
| Breast Cancer | CTCATACTACTGATACTGCTGGGTATAATGCAATGGAAGAAA | 953 |
| Pro-1637-Leu | GTGTGAGCAGGGAGAAGCCAGAATTGACAGCTTCAACAGAA | |
| CCA to CTA | AGGGTCAACAAAAGAATGTCCATGGTGGTGTCTGGCCT | |
| | AGGCCAGACACCACCATGGACATTCTTTTGTTGACCCTTTCT | 954 |
| | GTTGAAGCTGTCAATTCTGGCTTCTCCCTGCTCACACTTTCTT | |
| | CCATTGCATTATACCCAGCAGTATCAGTAGTATGAG | |
| | GGAGAAGCCAGAATTGA | 955 |
| | TCAATTCTGGCTTCTCC | 956 |
| Breast Cancer | GAGCAGGGAGAAGCCAGAATTGACAGCTTCAACAGAAAGGG | 957 |
| Met-1652-Ile | TCAACAAAAGAATGTCCATGGTGGTGTCTGGCCTGACCCCAG | |
| ATG to ATA | AAGAATTTGTGAGTGTATCCATATGTATCTCCCTAATG | |
| | CATTAGGGAGATACATATGGATACACTCACAAATTCTTCTGG | 958 |
| | GGTCAGGCCAGACACCACCATGGACATTCTTTTGTTGACCCT | |
| | TTCTGTTGAAGCTGTCAATTCTGGCTTCTCCCTGCTC | |
| | ATGTCCATGGTGGTGTC | 959 |
| | GACACCACCATGGACAT | 960 |
| Breast Cancer | CACTTCCTGATTTTGTTTTCAACTTCTAATCCTTTGAGTGTTTT | 961 |
| Glu-1694-Stop | TCATTCTGCAGATGCTGAGTTTGTGTGTGAACGGACACTGAA | |
| GAG to TAG | ATATTTTCTAGGAATTGCGGGAGGAAAATGGGTAG | |
| | CTACCCATTTTCCTCCCGCAATTCCTAGAAAATATTTCAGTGT | 962 |
| | CCGTTCACACACAAACTCAGCATCTGCAGAATGAAAAACACT | |
| | CAAAGGATTAGAAGTTGAAAACAAAATCAGGAAGTG | |
| | CAGATGCTGAGTTTGTG | 963 |
| | CACAAACTCAGCATCTG | 964 |
| Breast Cancer | CTGTTTTTCATTCTGCAGATGCTGAGTTTGTGTGTGAACGGA | 965 |
| Gly-1706-Glu | CACTGAAATATTTTCTAGGAATTGCGGGAGGAAAATGGGTAG | |
| GGA to GAA | TTAGCTATTTCTGTAAGTATAATACTATTTCTCCCCT | |
| | AGGGGAGAAATAGTATTATACTTACAGAAATAGCTAACTACC | 966 |
| | CATTTTCCTCCCGCAATTCCTAGAAAATATTTCAGTGTCCGTT | |
| | CACACACAAACTCAGCATCTGCAGAATGAAAAACAC | |
| | TTTTCTAGGAATTGCGG | 967 |
| | CCGCAATTCCTAGAAAA | 968 |
| Breast Cancer | TTCATTCTGCAGATGCTGAGTTTGTGTGTGAACGGACACTGA | 969 |
| Ala-1708-Glu | AATATTTTCTAGGAATTGCGGGAGGAAAATGGGTAGTTAGCT | |
| GCG to GAG | ATTTCTGTAAGTATAATACTATTTCTCCCCTCCTCCC | |
| | GGGAGGAGGGGAGAAATAGTATTATACTTACAGAAATAGCTA | 970 |
| | ACTACCCATTTTCCTCCCGCAATTCCTAGAAAATATTTCAGTG | |
| | TCCGTTCACACACAAACTCAGCATCTGCAGAATGAA | |
| | AGGAATTGCGGGAGGAA | 971 |
| | TTCCTCCCGCAATTCCT | 972 |
| Breast Cancer | CTGAGTTTGTGTGTGAACGGACACTGAAATATTTTCTAGGAAT | 973 |
| Val-1713-Ala | TGCGGGAGGAAAATGGGTAGTTAGCTATTTCTGTAAGTATAA | |
| GTA to GCA | TACTATTTCTCCCCTCCTCCCTTTAACACCTCAGAA | |
| | TTCTGAGGTGTTAAAGGGAGGAGGGGAGAAATAGTATTATAC | 974 |
| | TTACAGAAATAGCTAACTACCCATTTTCCTCCCGCAATTCCTA | |
| | GAAAATATTTCAGTGTCCGTTCACACACAAACTCAG | |
| | AAAATGGGTAGTTAGCT | 975 |
| | AGCTAACTACCCATTTT | 976 |
| Breast Cancer | AACGGACACTGAAATATTTTCTAGGAATTGCGGGAGGAAAAT | 977 |
| Trp-1718-Stop | GGGTAGTTAGCTATTTCTGTAAGTATAATACTATTTCTCCCCT | |
| TGG to TAG | CCTCCCTTTAACACCTCAGAATTGCATTTTTACACC | |
| | GGTGTAAAAATGCAATTCTGAGGTGTTAAAGGGAGGAGGGG | 978 |
| | AGAAATAGTATTATACTTACAGAAATAGCTAACTACCCATTTT | |
| | CCTCCCGCAATTCCTAGAAAATATTTCAGTGTCCGTT | |
| | CTATTTCTGTAAGTATA | 979 |
| | TATACTTACAGAAATAG | 980 |
| Breast Cancer | TTCTGCTGTATGTAACCTGTCTTTTCTATGATCTCTTTAGGGG | 981 |
| Glu-1725-Stop | TGACCCAGTCTATTAAAGAAAGAAAAATGCTGAATGAGGTAA | |
| GAA to TAA | GTACTTGATGTTACAAACTAACCAGAGATATTCATT | |
| | AATGAATATCTCTGGTTAGTTTGTAACATCAAGTACTTACCTC | 982 |
| | ATTCAGCATTTTTCTTTCTTTAATAGACTGGGTCACCCCTAAA | |
| | GAGATCATAGAAAAGACAGGTTACATACAGCAGAA | |
| | CTATTAAAGAAAGAAAA | 983 |
| | TTTTCTTTCTTTAATAG | 984 |
| Breast Cancer | TGTATGTAACCTGTCTTTTCTATGATCTCTTTAGGGGTGACCC | 985 |
| Lys-1727-Stop | AGTCTATTAAAGAAAGAAAAATGCTGAATGAGGTAAGTACTT | |
| AAA to TAA | GATGTTACAAACTAACCAGAGATATTCATTCAGTCA | |
| | TGACTGAATGAATATCTCTGGTTAGTTTGTAACATCAAGTACT | 986 |
| | TACCTCATTCAGCATTTTTCTTTCTTTAATAGACTGGGTCACC | |
| | CCTAAAGAGATCATAGAAAAGACAGGTTACATACA | |
| | AAGAAAGAAAAATGCTG | 987 |
| | CAGCATTTTTCTTTCTT | 988 |
| Breast Cancer | TCTTTCAGCATGATTTTGAAGTCAGAGGAGATGTGGTCAATG | 989 |
| Pro-1749-Arg | GAAGAAACCACCAAGGTCCAAAGCGAGCAAGAGAATCCCAG | |
| CCA to CGA | GACAGAAAGGTAAAGCTCCCTCCCTCAAGTTGACAAAA | |
| | TTTTGTCAACTTGAGGGAGGGAGCTTTACCTTTCTGTCCTGG | 990 |
| | GATTCTCTTGCTCGCTTTGGACCTTGGTGGTTTCTTCCATTGA | |
| | CCACATCTCCTCTGACTTCAAAATCATGCTGAAAGA | |
| | CCAAGGTCCAAAGCGAG | 991 |
| | CTCGCTTTGGACCTTGG | 992 |
| Breast Cancer | CAGCATGATTTTGAAGTCAGAGGAGATGTGGTCAATGGAAGA | 993 |
| Arg-1751-Stop | AACCACCAAGGTCCAAAGCGAGCAAGAGAATCCCAGGACAG | |
| CGA to TGA | AAAGGTAAAGCTCCCTCCCTCAAGTTGACAAAAATCTC | |
| | GAGATTTTTGTCAACTTGAGGGAGGGAGCTTTACCTTTCTGT | 994 |
| | CCTGGGATTCTCTTGCTCGCTTTGGACCTTGGTGGTTTCTTC | |
| | CATTGACCACATCTCCTCTGACTTCAAAATCATGCTG | |
| | GTCCAAAGCGAGCAAGA | 995 |
| | TCTTGCTCGCTTTGGAC | 996 |
| Breast Cancer | GTCAGAGGAGATGTGGTCAATGGAAGAAACCACCAAGGTCC | 997 |
| Gln-1756-Stop | AAAGCGAGCAAGAGAATCCCAGGACAGAAAGGTAAAGCTCC | |
| CAG to TAG | CTCCCTCAAGTTGACAAAAATCTCACCCCACCACTCTGT | |
| | ACAGAGTGGTGGGGTGAGATTTTTGTCAACTTGAGGGAGGG | 998 |
| | AGCTTTACCTTTCTGTCCTGGGATTCTCTTGCTCGCTTTGGAC | |
| | CTTGGTGGTTTCTTCCATTGACCACATCTCCTCTGAC | |
| | GAGAATCCCAGGACAGA | 999 |
| | TCTGTCCTGGGATTCTC | 1000 |
| Breast Cancer | CTCTCTTCTTCCAGATCTTCAGGGGGCTAGAAATCTGTTGCT | 1001 |
| Met-1775-Arg | ATGGGCCCTTCACCAACATGCCCACAGGTAAGAGCCTGGGA | |
| ATG to AGG | GAACCCCAGAGTTCCAGCACCAGCCTTTGTCTTACATA | |
| | TATGTAAGACAAAGGCTGGTGCTGGAACTCTGGGGTTCTCCC | 1002 |
| | AGGCTCTTACCTGTGGGCATGTTGGTGAAGGGCCCATAGCA | |
| | ACAGATTTCTAGCCCCCTGAAGATCTGGAAGAAGAGAG | |
| | CACCAACATGCCCACAG | 1003 |
| | CTGTGGGCATGTTGGTG | 1004 |
| Breast Cancer | AGTATGCAGATTACTGCAGTGATTTTACATCTAAATGTCCATT | 1005 |
| Trp-1782-Stop | TTAGATCAACTGGAATGGATGGTACAGCTGTGTGGTGCTTCT | |
| TGG to TGA | GTGGTGAAGGAGCTTTCATCATTCACCCTTGGCACA | |
| | TGTGCCAAGGGTGAATGATGAAAGCTCCTTCACCACAGAAGC | 1006 |
| | ACCACACAGCTGTACCATCCATTCCAGTTGATCTAAAATGGA | |
| | CATTTAGATGTAAAATCACTGCAGTAATCTGCATACT | |
| | CTGGAATGGATGGTACA | 1007 |
| | TGTACCATCCATTCCAG | 1008 |
| Breast Cancer | ATTACTGCAGTGATTTTACATCTAAATGTCCATTTTAGATCAA | 1009 |
| Gln-1785-His | CTGGAATGGATGGTACAGCTGTGTGGTGCTTCTGTGGTGAA | |
| CAG to CAT | GGAGCTTTCATCATTCACCCTTGGCACAGTAAGTATT | |
| | AATACTTACTGTGCCAAGGGTGAATGATGAAAGCTCCTTCAC | 1010 |
| | CACAGAAGCACCACACAGCTGTACCATCCATTCCAGTTGATC | |
| | TAAAATGGACATTTAGATGTAAAATCACTGCAGTAAT | |
| | ATGGTACAGCTGTGTGG | 1011 |
| | CCACACAGCTGTACCAT | 1012 |
| Breast Cancer | GTCCATTTTAGATCAACTGGAATGGATGGTACAGCTGTGTGG | 1013 |
| Glu-1794-Asp | TGCTTCTGTGGTGAAGGAGCTTTCATCATTCACCCTTGGCAC | |
| GAG to GAT | AGTAAGTATTGGGTGCCCTGTCAGAGAGGGAGGACAC | |
| | GTGTCCTCCCTCTCTGACAGGGCACCCAATACTTACTGTGCC | 1014 |
| | AAGGGTGAATGATGAAAGCTCCTTCACCACAGAAGCACCACA | |
| | CAGCTGTACCATCCATTCCAGTTGATCTAAAATGGAC | |
| | GTGAAGGAGCTTTCATC | 1015 |
| | GATGAAAGCTCCTTCAC | 1016 |
| Breast Cancer | CTCTGCTTGTGTTCTCTGTCTCCAGCAATTGGGCAGATGTGT | 1017 |
| Arg-1835-Stop | GAGGCACCTGTGGTGACCCGAGAGTGGGTGTTGGACAGTGT | |
| CGA to TGA | AGCACTCTACCAGTGCCAGGAGCTGGACACCTACCTGA | |
| | TCAGGTAGGTGTCCAGCTCCTGGCACTGGTAGAGTGCTACA | 1018 |
| | CTGTCCAACACCCACTCTCGGGTCACCACAGGTGCCTCACA | |
| | CATCTGCCCAATTGCTGGAGACAGAGAACACAAGCAGAG | |
| | TGGTGACCCGAGAGTGG | 1019 |
| | CCACTCTCGGGTCACCA | 1020 |
| Breast Cancer | TTGTGTTCTCTGTCTCCAGCAATTGGGCAGATGTGTGAGGCA | 1021 |
| Trp-1837-Arg | CCTGTGGTGACCCGAGAGTGGGTGTTGGACAGTGTAGCACT | |
| TGG to CGG | CTACCAGTGCCAGGAGCTGGACACCTACCTGATACCCC | |
| | GGGGTATCAGGTAGGTGTCCAGCTCCTGGCACTGGTAGAGT | 1022 |
| | GCTACACTGTCCAACACCCACTCTCGGGTCACCACAGGTGC | |
| | CTCACACATCTGCCCAATTGCTGGAGACAGAGAACACAA | |
| | CCCGAGAGTGGGTGTTG | 1023 |
| | CAACACCCACTCTCGGG | 1024 |
| Breast Cancer | TGTGTTCTCTGTCTCCAGCAATTGGGCAGATGTGTGAGGCAC | 1025 |
| Trp-1837-Stop | CTGTGGTGACCCGAGAGTGGGTGTTGGACAGTGTAGCACTC | |
| TGG to TAG | TACCAGTGCCAGGAGCTGGACACCTACCTGATACCCCA | |
| | TGGGGTATCAGGTAGGTGTCCAGCTCCTGGCACTGGTAGAG | 1026 |
| | TGCTACACTGTCCAACACCCACTCTCGGGTCACCACAGGTG | |
| | CCTCACACATCTGCCCAATTGCTGGAGACAGAGAACACA | |
| | CCGAGAGTGGGTGTTGG | 1027 |
| | CCAACACCCACTCTCGG | 1028 |

TABLE 10
BRCA2 Mutations and Genome-Correcting Oligos

| Clinical Phenotype & | | SEQ ID |
|---|---|---|
| Mutation | Correcting Oligos | NO: |
| Breast cancer | GTTAAAACTAAGGTGGGATTTTTTTTTTAAATAGATTTAGGAC | 1029 |
| PHE32LEU | CAATAAGTCTTAATTGGTTTGAAGAACTTTCTTCAGAAGCTCC | |
| TTT to CTT | ACCCTATAATTCTGAACCTGCAGAAGAATCTGAAC | |
| | GTTCAGATTCTTCTGCAGGTTCAGAATTATAGGGTGGAGCTT | 1030 |
| | CTGAAGAAAGTTCTTCAAACCAATTAAGACTTATTGGTCCTAA | |
| | ATCTATTTAAAAAAAAAATCCCACCTTAGTTTTAAC | |
| | TTAATTGGTTTGAAGAA | 1031 |
| | TTCTTCAAACCAATTAA | 1032 |
| Breast cancer | TAGATTTAGGACCAATAAGTCTTAATTGGTTTGAAGAACTTTC | 1033 |
| TYR42CYS | TTCAGAAGCTCCACCCTATAATTCTGAACCTGCAGAAGAATC | |
| TAT to TGT | TGAACATAAAAACAACAATTACGAACCAAACCTATT | |
| | AATAGGTTTGGTTCGTAATTGTTGTTTTTATGTTCAGATTCTTC | 1034 |
| | TGCAGGTTCAGAATTATAGGGTGGAGCTTCTGAAGAAAGTTC | |
| | TTCAAACCAATTAAGACTTATTGGTCCTAAATCTA | |
| | TCCACCCTATAATTCTG | 1035 |
| | CAGAATTATAGGGTGGA | 1036 |
| Breast cancer | AAGAACTTTCTTCAGAAGCTCCACCCTATAATTCTGAACCTGC | 1037 |
| LYS53ARG | AGAAGAATCTGAACATAAAAACAACAATTACGAACCAAACCTA | |
| AAA to AGA | TTTAAAACTCCACAAAGGAAACCATCTTATAATCA | |
| | TGATTATAAGATGGTTTCCTTTGTGGAGTTTTAAATAGGTTTG | 1038 |
| | GTTCGTAATTGTTGTTTTTATGTTCAGATTCTTCTGCAGGTTC | |
| | AGAATTATAGGGTGGAGCTTCTGAAGAAAGTTCTT | |
| | TGAACATAAAAACAACA | 1039 |
| | TGTTGTTTTTATGTTCA | 1040 |
| Breast cancer | CTATTTAAAACTCCACAAAGGAAACCATCTTATAATCAGCTGG | 1041 |
| Phe81Leu | CTTCAACTCCAATAATATTCAAAGAGCAAGGGCTGACTCTGC | |
| TTC to CTC | CGCTGTACCAATCTCCTGTAAAAGAATTAGATAAAT | |
| | ATTTATCTAATTCTTTTACAGGAGATTGGTACAGCGGCAGAGT | 1042 |
| | CAGCCCTTGCTCTTTGAATATTATTGGAGTTGAAGCCAGCTG | |
| | ATTATAAGATGGTTTCCTTTGTGGAGTTTTAAATAG | |
| | CAATAATATTCAAAGAG | 1043 |
| | CTCTTTGAATATTATTG | 1044 |
| Breast cancer | GTCAGACACCAAAACATATTTCTGAAAGTCTAGGAGCTGAGG | 1045 |
| TRP194TERM | TGGATCCTGATATGTCTTGGTCAAGTTCTTTAGCTACACCACC | |
| TGG to TAG | CACCCTTAGTTCTACTGTGCTCATAGGTAATAATAG | |
| | CTATTATTACCTATGAGCACAGTAGAACTAAGGGTGGGTGGT | 1046 |
| | GTAGCTAAAGAACTTGACCAAGACATATCAGGATCCACCTCA | |
| | GCTCCTAGACTTTCAGAAATATGTTTTGGTGTCTGAC | |
| | TATGTCTTGGTCAAGTT | 1047 |
| | AACTTGACCAAGACATA | 1048 |
| Breast cancer | CTGAAAGTCTAGGAGCTGAGGTGGATCCTGATATGTCTTGGT | 1049 |
| PRO201ARG | CAAGTTCTTTAGCTACACCACCCACCCTTAGTTCTACTGTGCT | |
| CCA to CGA | CATAGGTAATAATAGCAAATGTGTATTTACAAGAAA | |
| | TTTCTTGTAAATACACATTTGCTATTATTACCTATGAGCACAGT | 1050 |
| | AGAACTAAGGGTGGGTGGTGTAGCTAAAGAACTTGACCAAG | |
| | ACATATCAGGATCCACCTCAGCTCCTAGACTTTCAG | |
| | AGCTACACCACCCACCC | 1051 |
| | GGGTGGGTGGTGTAGCT | 1052 |
| Breast cancer | ACAATACACATAAATTTTTATCTTACAGTCAGAAATGAAGAAG | 1053 |
| Pro222Ser | CATCTGAAACTGTATTTCCTCATGATACTACTGCTGTAAGTAA | |
| CCT to TCT | ATATGACATTGATTAGACTGTTGAAATTGCTAACA | |
| | TGTTAGCAATTTCAACAGTCTAATCAATGTCATATTTACTTACA | 1054 |
| | GCAGTAGTATCATGAGGAAATACAGTTTCAGATGCTTCTTCAT | |
| | TTCTGACTGTAAGATAAAAATTTATGTGTATTGT | |
| | CTGTATTTCCTCATGAT | 1055 |
| | ATCATGAGGAAATACAG | 1056 |
| Breast cancer | AATGGTCTCAACTAACCCTTTCAGGTCTAAATGGAGCCCAGA | 1057 |
| Leu-414-Term | TGGAGAAAATACCCCTATTGCATATTTCTTCATGTGACCAAAA | |
| TTG to TAG | TATTTCAGAAAAAGACCTATTAGACACAGAGAACAA | |
| | TTGTTCTCTGTGTCTAATAGGTCTTTTTCTGAAATATTTTGGTC | 1058 |
| | ACATGAAGAAATATGCAATAGGGGTATTTTCTCCATCTGGGC | |
| | TCCATTTAGACCTGAAAGGGTTAGTTGAGACCATT | |
| | ACCCCTATTGCATATTT | 1059 |
| | AAATATGCAATAGGGGT | 1060 |
| Breast cancer, male | AGCCTCTGAAAGTGGACTGGAAATACATACTGTTTGCTCACA | 1061 |
| Cys554Trp | GAAGGAGGACTCCTTATGTCCAAATTTAATTGATAATGGAAG | |
| TGT to TGG | CTGGCCAGCCACCACCACACAGAATTCTGTAGCTTTG | |
| | CAAAGCTACAGAATTCTGTGTGGTGGTGGCTGGCCAGCTTC | 1062 |
| | CATTATCAATTAAATTTGGACATAAGGAGTCCTCCTTCTGTGA | |
| | GCAAACAGTATGTATTTCCAGTCCACTTTCAGAGGCT | |
| | TCCTTATGTCCAAATTT | 1063 |
| | AAATTTGGACATAAGGA | 1064 |
| Breast cancer | AACTCTACCATGGTTTTATATGGAGACACAGGTGATAAACAA | 1065 |
| Lys944Term | GCAACCCAAGTGTCAATTAAAAAAGATTTGGTTTATGTTCTTG | |
| AAA to TAA | CAGAGGAGAACAAAAATAGTGTAAAGCAGCATATAA | |
| | TTATATGCTGCTTTACACTATTTTTGTTCTCCTCTGCAAGAAC | 1066 |
| | ATAAACCAAATCTTTTTTAATTGACACTTGGGTTGCTTGTTTAT | |
| | CACCTGTGTCTCCATATAAAACCATGGTAGAGTT | |
| | TGTCAATTAAAAAAGAT | 1067 |
| | ATCTTTTTTAATTGACA | 1068 |
| Breast cancer, male | ATGACTACTGGCACTTTTGTTGAAGAAATTACTGAAAATTACA | 1069 |
| Glu1320Term | AGAGAAATACTGAAAATGAAGATAACAAATATACTGCTGCCA | |
| GAA to TAA | GTAGAAATTCTCATAACTTAGAATTTGATGGCAGTG | |
| | CACTGCCATCAAATTCTAAGTTATGAGAATTTCTACTGGCAGC | 1070 |
| | AGTATATTTGTTATCTTCATTTTCAGTATTTCTCTTGTAATTTTC | |
| | AGTAATTTCTTCAACAAAAGTGCCAGTAGTCAT | |
| | CTGAAAATGAAGATAAC | 1071 |
| | GTTATCTTCATTTTCAG | 1072 |
| Breast cancer | CATGAAACAATTAAAAAAGTGAAAGACATATTTACAGACAGTT | 1073 |
| Glu1876Term | TCAGTAAAGTAATTAAGGAAAACAACGAGAATAAATCAAAAAT | |
| GAA to TAA | TTGCCAAACGAAAATTATGGCAGGTTGTTACGAGG | |
| | CCTCGTAACAACCTGCCATAATTTTCGTTTGGCAAATTTTTGA | 1074 |
| | TTTATTCTCGTTGTTTTCCTTAATTACTTTACTGAAACTGTCTG | |
| | TAAATATGTCTTTCACTTTTTTAATTGTTTCATG | |
| | TAATTAAGGAAAACAAC | 1075 |
| | GTTGTTTTCCTTAATTA | 1076 |
| Breast cancer | TGAAAGACATATTTACAGACAGTTTCAGTAAAGTAATTAAGGA | 1077 |
| Ser1882Term | AAACAACGAGAATAAATCAAAAATTTGCCAAACGAAAATTATG | |
| TCA to TAA | GCAGGTTGTTACGAGGCATTGGATGATTCAGAGGA | |
| | TCCTCTGAATCATCCAATGCCTCGTAACAACCTGCCATAATTT | 1078 |
| | TCGTTTGGCAAATTTTTGATTTATTCTCGTTGTTTTCCTTAATT | |
| | ACTTTACTGAAACTGTCTGTAAATATGTCTTTCA | |
| | GAATAAATCAAAAATTT | 1079 |
| | AAATTTTTGATTTATTC | 1080 |
| Breast cancer | AACCAAAATATGTCTGGATTGGAGAAAGTTTCTAAAATATCAC | 1081 |
| Glu1953Term | CTTGTGATGTTAGTTTGGAAACTTCAGATATATGTAAATGTAG | |
| GAA to TAA | TATAGGGAAGCTTCATAAGTCAGTCTCATCTGCAA | |
| | TTGCAGATGAGACTGACTTATGAAGCTTCCCTATACTACATTT | 1082 |
| | ACATATATCTGAAGTTTCCAAACTAACATCACAAGGTGATATT | |
| | TTAGAAACTTTCTCCAATCCAGACATATTTTGGTT | |
| | TTAGTTTGGAAACTTCA | 1083 |
| | TGAAGTTTCCAAACTAA | 1084 |
| Breast cancer | TTAGTTTGGAAACTTCAGATATATGTAAATGTAGTATAGGGAA | 1085 |
| Ser1970Term | GCTTCATAAGTCAGTCTCATCTGCAAATACTTGTGGGATTTTT | |
| TCA to TAA | AGCACAGCAAGTGGAAAATCTGTCCAGGTATCAGA | |
| | TCTGATACCTGGACAGATTTTCCACTTGCTGTGCTAAAAATCC | 1086 |
| | CACAAGTATTTGCAGATGAGACTGACTTATGAAGCTTCCCTAT | |
| | ACTACATTTACATATATCTGAAGTTTCCAAACTAA | |
| | GTCAGTCTCATCTGCAA | 1087 |
| | TTGCAGATGAGACTGAC | 1088 |
| Breast cancer | AAGTCAGTCTCATCTGCAAATACTTGTGGGATTTTTAGCACAG | 1089 |
| Gln1987Term | CAAGTGGAAAATCTGTCCAGGTATCAGATGCTTCATTACAAA | |
| CAG | ACGCAAGACAAGTGTTTTCTGAAATAGAAGATAGTA | |
| | TACTATCTTCTATTTCAGAAAACACTTGTCTTGCGTTTTGTAAT | 1090 |
| | GAAGCATCTGATACCTGGACAGATTTTCCACTTGCTGTGCTA | |
| | AAAATCCCACAAGTATTTGCAGATGAGACTGACTT | |
| | AATCTGTCCAGGTATCA | 1091 |
| | TGATACCTGGACAGATT | 1092 |
| Breast cancer | AAAATAAGATTAATGACAATGAGATTCATCAGTTTAACAAAAA | 1093 |
| Ala2466Val | CAACTCCAATCAAGCAGCAGCTGTAACTTTCACAAAGTGTGA | |
| GCA to GTA | AGAAGAACCTTTAGGTATTGTATGACAATTTGTGTG | |
| | CACACAAATTGTCATACAATACCTAAAGGTTCTTCTTCACACT | 1094 |
| | TTGTGAAAGTTACAGCTGCTGCTTGATTGGAGTTGTTTTTGTT | |
| | AAACTGATGAATCTCATTGTCATTAATCTTATTTT | |
| | TCAAGCAGCAGCTGTAA | 1095 |
| | TTACAGCTGCTGCTTGA | 1096 |
| Breast cancer | AGGCAACGCGTCTTTCCACAGCCAGGCAGTCTGTATCTTGCA | 1097 |
| Arg2 | AAAACATCCACTCTGCCTCGAATCTCTCTGAAAGCAGCAGTA | |
| | GGAGGCCAAGTCCCCTCTGCGTGTCCTCATAAACAGG | |
| | CCTGTTTATGAGGACACGCAGAGGGGACTTGGCCTCCTACT | 1098 |
| | GCTGCTTTCAGAGAGATTCGAGGCAGAGTGGATGTTTTTGCA | |
| | AGATACAGACTGCCTGGCTGTGGAAAGACGCGTTGCCT | |
| | CTCTGCCTCGAATCTCT | 1099 |
| | AGAGATTCGAGGCAGAG | 1100 |
| Breast cancer | ATTTCATTGAGCGCAAATATATCTGAAACTTCTAGCAATAAAA | 1101 |
| Gln2714Term | CTAGTAGTGCAGATACCCAAAAAGTGGCCATTATTGAACTTA | |
| CAA to TAA | CAGATGGGTGGTATGCTGTTAAGGCCCAGTTAGATC | |
| | GATCTAACTGGGCCTTAACAGCATACCACCCATCTGTAAGTT | 1102 |
| | CAATAATGGCCACTTTTTGGGTATCTGCACTACTAGTTTTATT | |
| | GCTAGAAGTTTCAGATATATTTGCGCTCAATGAAAT | |
| | CAGATACCCAAAAAGTG | 1103 |
| | CACTTTTTGGGTATCTG | 1104 |
| Breast cancer | CAGAACTGGTGGGCTCTCCTGATGCCTGTACACCTCTTGAAG | 1105 |
| Leu2776Term | CCCCAGAATCTCTTATGTTAAAGGTAAATTAATTTGCACTCTT | |
| TTA to TGA | GGTAAAAATCAGTCATTGATTCAGTTAAATTCTAGA | |
| | TCTAGAATTTAACTGAATCAATGACTGATTTTTACCAAGAGTG | 1106 |
| | CAAATTAATTTACCTTTAACATAAGAGATTCTGGGGCTTCAAG | |
| | AGGTGTACAGGCATCAGGAGAGCCCACCAGTTCTG | |
| | TCTTATGTTAAAGATTT | 1107 |
| | AAATCTTTAACATAAGA | 1108 |
| Breast cancer | CCTTTTGTTTTCTTAGAAAACACAACAAAACCATATTTACCATC | 1109 |
| Gln2893Term | ACGTGCACTAACAAGACAGCAAGTTCGTGCTTTGCAAGATGG | |
| CAG to TAG | TGCAGAGCTTTATGAAGCAGTGAAGAATGCAGCAG | |
| | CTGCTGCATTCTTCACTGCTTCATAAAGCTCTGCACCATCTTG | 1110 |
| | CAAAGCACGAACTTGCTGTCTTGTTAGTGCACGTGATGGTAA | |
| | ATATGGTTTTGTTGTGTTTTCTAAGAAAACAAAAGG | |
| | TAACAAGACAGCAAGTT | 1111 |
| | AACTTGCTGTCTTGTTA | 1112 |
| Breast cancer | AATCACAGGCAAATGTTGAATGATAAGAAACAAGCTCAGATC | 1113 |
| Ala2951Thr | CAGTTGGAAATTAGGAAGGCCATGGAATCTGCTGAACAAAAG | |
| GCC to ACC | GAACAAGGTTTATCAAGGGATGTCACAACCGTGTGGA | |
| | TCCACACGGTTGTGACATCCCTTGATAAACCTTGTTCCTTTTG | 1114 |
| | TTCAGCAGATTCCATGGCCTTCCTAATTTCCAACTGGATCTGA | |
| | GCTTGTTTCTTATCATTCAACATTTGCCTGTGATT | |
| | TTAGGAAGGCCATGGAA | 1115 |
| | TTCCATGGCCTTCCTAA | 1116 |
| Breast cancer | ACAATTTACTGGCAATAAAGTTTTGGATAGACCTTAATGAGGA | 1117 |
| Met3118Th | CATTATTAAGCCTCATATGTTAATTGCTGCAAGCAACCTCCAG | |
| | TGGCGACCAGAATCCAAATCAGGCCTTCTTACTTT | |
| | AAAGTAAGAAGGCCTGATTTGGATTCTGGTCGCCACTGGAG | 1118 |
| | GTTGCTTGCAGCAATTAACATATGAGGCTTAATAATGTCCTCA | |
| | TTAAGGTCTATCCAAAACTTTATTGCCAGTAAATTGT | |
| | GCCTCATATGTTAATTG | 1119 |
| | CAATTAACATATGAGGC | 1120 |
| Breast cancer | GACTGAAACGACGTTGTACTACATCTCTGATCAAAGAACAGG | 1121 |
| Thr3401Met | AGAGTTCCCAGGCCAGTACGGAAGAATGTGAGAAAAATAAG | |
| ACG to ATG | CAGGACACAATTACAACTAAAAAATATATCTAAGCATT | |
| | AATGCTTAGATATATTTTTTAGTTGTAATTGTGTCCTGCTTATT | 1122 |
| | TTTCTCACATTCTTCCGTACTGGCCTGGGAACTCTCCTGTTCT | |
| | TTGATCAGAGATGTAGTACAACGTCGTTTCAGTC | |
| | GGCCAGTACGGAAGAAT | 1123 |
| | ATTCTTCCGTACTGGCC | 1124 |
| Breast cancer | AAAGAACAGGAGAGTTCCCAGGCCAGTACGGAAGAATGTGA | 1125 |
| Ile3412Val | GAAAAATAAGCAGGACACAATTACAACTAAAAAATATATCTAA | |
| ATT to GTT | GCATTTGCAAAGGCGACAATAAATTATTGACGCTTAA | |
| | TTAAGCGTCAATAATTTATTGTCGCCTTTGCAAATGCTTAGAT | 1126 |
| | ATATTTTTTAGTTGTAATTGTGTCCTGCTTATTTTTCTCACATT | |
| | CTTCCGTACTGGCCTGGGAACTCTCCTGTTCTTT | |
| | AGGACACAATTACAACT | 1127 |
| | AGTTGTAATTGTGTCCT | 1128 |

### EXAMPLE 10

### Cystic Fibrosis—CFTR

Cystic fibrosis is a lethal disease affecting approximately one in 2,500 live Caucasian births and is the most common autosomal recessive disease in Caucasians. Patients with this disease have reduced chloride ion permeability in the secretory and absorptive cells of organs with epithelial cell linings, including the airways, pancreas, intestine, sweat glands and male genital tract. This, in turn, reduces the transport of water across the epithelia. The lungs and the GI tract are the predominant organ systems affected in this disease and the pathology is characterized by blocking of the respiratory and GI tracts with viscous mucus. The chloride impermeability in affected tissues is due to mutations in a specific chloride channel, the cystic fibrosis transmembrane conductance regulator protein (CFTR), which prevents normal passage of chloride ions through the cell membrane (Welsh et al., Neuron, 8:821-829 (1992)). Damage to the lungs due to mucus blockage, frequent bacterial infections and inflammation is the primary cause of morbidity and mortality in CF patients and, although maintenance therapy has improved the quality of patients' lives, the median age at death is still only around 30 years. There is no effective treatment for the disease, and therapeutic research is focused on gene therapy using exogenous transgenes in viral vectors and/or activating the defective or other chloride channels in the cell membrane to normalize chloride permeability (Tizzano et al., J. Pediat., 120:337-349 (1992)). However, the death of a teenage patient treated with an adenovirus vector carrying an exogenous CFTR gene in clinical trials in the late 1990's has impacted this area of research.

The oligonucleotides of the invention for correction of the CFTR gene are attached as a table.

TABLE 12
CFTR Mutations and Genome-Correcting Oligos

| Clinical Phenotype & | | SEQ ID |
|---|---|---|
| Mutation | Correcting Oligos | NO: |
| Cystic fibrosis | AAGGATACAGACAGCGCCTGGAATTGTCAGACATATACCAAA | 1129 |
| Ala46Asp | TCCCTTCTGTTGATTCTGCTGACAATCTATCTGAAAAATTGGA | |
| GCT to GAT | AAGGTATGTTCATGTACATTGTTTAGTTGAAGAGAG | |
| | CTCTCTTCAACTAAACAATGTACATGAACATACCTTTCCAATT | 1130 |
| | TTTCAGATAGATTGTCAGCAGAATCAACAGAAGGGATTTGGT | |
| | ATATGTCTGACAATTCCAGGCGCTGTCTGTATCCTT | |
| | TGATTCTGCTGACAATC | 1131 |
| | GATTGTCAGCAGAATCA | 1132 |
| Cystic fibrosis | AGCGCCTGGAATTGTCAGACATATACCAAATCCCTTCTGTTG | 1133 |
| Ser50Tyr | ATTCTGCTGACAATCTATCTGAAAAATTGGAAAGGTATGTTCA | |
| TCT to TAT | TGTACATTGTTTAGTTGAAGAGAGAAATTCATATTA | |
| | TAATATGAATTTCTCTCTTCAACTAAACAATGTACATGAACATA | 1134 |
| | CCTTTCCAATTTTTCAGATAGATTGTCAGCAGAATCAACAGAA | |
| | GGGATTTGGTATATGTCTGACAATTCCAGGCGCT | |
| | CAATCTATCTGAAAAAT | 1135 |
| | ATTTTTCAGATAGATTG | 1136 |
| Congenital absence | AGGACAACTAAAATATTTGCACATGCAACTTATTGGTCCCACT | 1137 |
| of vas deferens | TTTTATTCTTTTGCAGAGAATGGGATAGAGAGCTGGCTTCAAA | |
| Glu56Lys | GAAAAATCCTAAACTCATTAATGCCCTTCGGCGAT | |
| GAA-AAA | | |
| | ATCGCCGAAGGGCATTAATGAGTTTAGGATTTTTCTTTGAAG | 1138 |
| | CCAGCTCTCTATCCCATTCTCTGCAAAAGAATAAAAAGTGGG | |
| | ACCAATAAGTTGCATGTGCAAATATTTTAGTTGTCCT | |
| | TTTGCAGAGAATGGGAT | 1139 |
| | ATCCCATTCTCTGCAAA | 1140 |
| Cystic fibrosis | AGGACAACTAAAATATTTGCACATGCAACTTATTGGTCCCACT | 1141 |
| Trp57Gly | TTTTATTCTTTTGCAGAGAATGGGATAGAGAGCTGGCTTCAAA | |
| TGG to GGG | GAAAAATCCTAAACTCATTAATGCCCTTCGGCGAT | |
| | ATCGCCGAAGGGCATTAATGAGTTTAGGATTTTTCTTTGAAG | 1142 |
| | CCAGCTCTCTATCCCATTCTCTGCAAAAGAATAAAAAGTGGG | |
| | ACCAATAAGTTGCATGTGCAAATATTTTAGTTGTCCT | |
| | TTTGCAGAGAATGGGAT | 1143 |
| | ATCCCATTCTCTGCAAA | 1144 |
| Cystic fibrosis | AACTAAAATATTTGCACATGCAACTTATTGGTCCCACTTTTTAT | 1145 |
| Trp57Term | TCTTTTGCAGAGAATGGGATAGAGAGCTGGCTTCAAAGAAAA | |
| TGG to TGA | ATCCTAAACTCATTAATGCCCTTCGGCGATGTTTT | |
| | AAAACATCGCCGAAGGGCATTAATGAGTTTAGGATTTTTCTTT | 1146 |
| | GAAGCCAGCTCTCTATCCCATTCTCTGCAAAAGAATAAAAAG | |
| | TGGGACCAATAAGTTGCATGTGCAAATATTTTAGTT | |
| | AGAGAATGGGATAGAGA | 1147 |
| | TCTCTATCCCATTCTCT | 1148 |
| Congenital absence | ACTAAAATATTTGCACATGCAACTTATTGGTCCCACTTTTTATT | 1149 |
| of vas deferens | CTTTTGCAGAGAATGGGATAGAGAGCTGGCTTCAAAGAAAAA | |
| Asp58Asn | TCCTAAACTCATTAATGCCCTTCGGCGATGTTTTT | |
| GAT to AAT | | |
| | AAAAACATCGCCGAAGGGCATTAATGAGTTTAGGATTTTTCTT | 1150 |
| | TGAAGCCAGCTCTCTATCCCATTCTCTGCAAAAGAATAAAAA | |
| | GTGGGACCAATAAGTTGCATGTGCAAATATTTTAGT | |
| | GAGAATGGGATAGAGAG | 1151 |
| | CTCTCTATCCCATTCTC | 1152 |
| Cystic fibrosis | ATATTTGCACATGCAACTTATTGGTCCCACTTTTTATTCTTTTG | 1153 |
| Glu60Term | CAGAGAATGGGATAGAGAGCTGGCTTCAAAGAAAAATCCTAA | |
| GAG to TAG | ACTCATTAATGCCCTTCGGCGATGTTTTTTCTGGA | |
| | TCCAGAAAAAACATCGCCGAAGGGCATTAATGAGTTTAGGAT | 1154 |
| | TTTTCTTTGAAGCCAGCTCTCTATCCCATTCTCTGCAAAAGAA | |
| | TAAAAAGTGGGACCAATAAGTTGCATGTGCAAATAT | |
| | GGGATAGAGAGCTGGCT | 1155 |
| | AGCCAGCTCTCTATCCC | 1156 |
| Cystic fibrosis | GGTCCCACTTTTTATTCTTTTGCAGAGAATGGGATAGAGAGC | 1157 |
| | TGGCTTCAAAGAAAAATCCTAAACTCATTAATGCCCTTCGGC | |
| | GATGTTTTTTCTGGAGATTTATGTTCTATGGAATCTT | |
| | AAGATTCCATAGAACATAAATCTCCAGAAAAAACATCGCCGA | 1158 |
| | AGGGCATTAATGAGTTTAGGATTTTTCTTTGAAGCCAGCTCTC | |
| | TATCCCATTCTCTGCAAAAGAATAAAAAGTGGGACC | |
| | GAAAAATCCTAAACTCA | 1159 |
| | TGAGTTTAGGATTTTTC | 1160 |
| Cystic fibrosis | TGCAGAGAATGGGATAGAGAGCTGGCTTCAAAGAAAAATCCT | 1161 |
| Arg74Trp | AAACTCATTAATGCCCTTCGGCGATGTTTTTTCTGGAGATTTA | |
| CGG to TGG | TGTTCTATGGAATCTTTTTATATTTAGGGGTAAGGA | |
| | TCCTTACCCCTAAATATAAAAAGATTCCATAGAACATAAATCT | 1162 |
| | CCAGAAAAAACATCGCCGAAGGGCATTAATGAGTTTAGGATT | |
| | TTTCTTTGAAGCCAGCTCTCTATCCCATTCTCTGCA | |
| | ATGCCCTTCGGCGATGT | 1163 |
| | ACATCGCCGAAGGGCAT | 1164 |
| Congenital absence | GAGAATGGGATAGAGAGCTGGCTTCAAAGAAAAATCCTAAAC | 1165 |
| of vas deferens | TCATTAATGCCCTTCGGCGATGTTTTTTCTGGAGATTTATGTT | |
| ARG75GLN | CTATGGAATCTTTTTATATTTAGGGGTAAGGATCTC | |
| CGA | | |
| | GAGATCCTTACCCCTAAATATAAAAAGATTCCATAGAACATAA | 1166 |
| | ATCTCCAGAAAAAACATCGCCGAAGGGCATTAATGAGTTTAG | |
| | GATTTTTCTTTGAAGCCAGCTCTCTATCCCATTCTC | |
| | CCTTCGGCGATGTTTTT | 1167 |
| | AAAAACATCGCCGAAGG | 1168 |
| Cystic fibrosis | GAGAATGGGATAGAGAGCTGGCTTCAAAGAAAAATCCTAAAC | 1169 |
| Arg75Leu | TCATTAATGCCCTTCGGCGATGTTTTTTCTGGAGATTTATGTT | |
| CGA to CTA | CTATGGAATCTTTTTATATTTAGGGGTAAGGATCTC | |
| | GAGATCCTTACCCCTAAATATAAAAAGATTCCATAGAACATAA | 1170 |
| | ATCTCCAGAAAAAACATCGCCGAAGGGCATTAATGAGTTTAG | |
| | GATTTTTCTTTGAAGCCAGCTCTCTATCCCATTCTC | |
| | CCTTCGGCGATGTTTTT | 1171 |
| | AAAAACATCGCCGAAGG | 1172 |
| Cystic fibrosis | AGAGAATGGGATAGAGAGCTGGCTTCAAAGAAAAATCCTAAA | 1173 |
| Arg75Term | CTCATTAATGCCCTTCGGCGATGTTTTTTCTGGAGATTTATGT | |
| CGA to TGA | TCTATGGAATCTTTTTATATTTAGGGGTAAGGATCT | |
| | AGATCCTTACCCCTAAATATAAAAAGATTCCATAGAACATAAA | 1174 |
| | TCTCCAGAAAAAACATCGCCGAAGGGCATTAATGAGTTTAGG | |
| | ATTTTTCTTTGAAGCCAGCTCTCTATCCCATTCTCT | |
| | CCCTTCGGCGATGTTTT | 1175 |
| | AAAACATCGCCGAAGGG | 1176 |
| Cystic fibrosis | AAAATCCTAAACTCATTAATGCCCTTCGGCGATGTTTTTTCTG | 1177 |
| Gly85Glu | GAGATTTATGTTCTATGGAATCTTTTTATATTTAGGGGTAAGG | |
| GGA to GAA | ATCTCATTTGTACATTCATTATGTATCACATAACT | |
| | AGTTATGTGATACATAATGAATGTACAAATGAGATCCTTACCC | 1178 |
| | CTAAATATAAAAAGATTCCATAGAACATAAATCTCCAGAAAAA | |
| | ACATCGCCGAAGGGCATTAATGAGTTTAGGATTTT | |
| | GTTCTATGGAATCTTTT | 1179 |
| | AAAAGATTCCATAGAAC | 1180 |
| Cystic fibrosis | AAAATCCTAAACTCATTAATGCCCTTCGGCGATGTTTTTTCTG | 1181 |
| G | GAGATTTATGTTCTATGGAATCTTTTTATATTTAGGGGTAAGG | |
| | ATCTCATTTGTACATTCATTATGTATCACATAACT | |
| | AGTTATGTGATACATAATGAATGTACAAATGAGATCCTTACCC | 1182 |
| | CTAAATATAAAAAGATTCCATAGAACATAAATCTCCAGAAAAA | |
| | ACATCGCCGAAGGGCATTAATGAGTTTAGGATTTT | |
| | GTTCTATGGAATCTTTT | 1183 |
| | AAAAGATTCCATAGAAC | 1184 |
| Cystic fibrosis | AACTCATTAATGCCCTTCGGCGATGTTTTTTCTGGAGATTTAT | 1185 |
| Leu88S | GTTCTATGGAATCTTTTTATATTTAGGGGTAAGGATCTCATTT | |
| | GTACATTCATTATGTATCACATAACTATATGCATT | |
| | AATGCATATAGTTATGTGATACATAATGAATGTACAAATGAGA | 1186 |
| | TCCTTACCCCTAAATATAAAAAGATTCCATAGAACATAAATCT | |
| | CCAGAAAAAACATCGCCGAAGGGCATTAATGAGTT | |
| | AATCTTTTTATATTTAG | 1187 |
| | CTAAATATAAAAAGATT | 1188 |
| Cystic fibrosis | CCTAAACTCATTAATGCCCTTCGGCGATGTTTTTTCTGGAGAT | 1189 |
| Phe87Leu | TTATGTTCTATGGAATCTTTTTATATTTAGGGGTAAGGATCTC | |
| TTT to CTT | ATTTGTACATTCATTATGTATCACATAACTATATG | |
| | CATATAGTTATGTGATACATAATGAATGTACAAATGAGATCCT | 1190 |
| | TACCCCTAAATATAAAAAGATTCCATAGAACATAAATCTCCAG | |
| | AAAAAACATCGCCGAAGGGCATTAATGAGTTTAGG | |
| | ATGGAATCTTTTTATAT | 1191 |
| | ATATAAAAAGATTCCAT | 1192 |
| Cystic fibrosis | AACTCATTAATGCCCTTCGGCGATGTTTTTTCTGGAGATTTAT | 1193 |
| Leu88Term | GTTCTATGGAATCTTTTTATATTTAGGGGTAAGGATCTCATTT | |
| TTA to TGA | GTACATTCATTATGTATCACATAACTATATGCATT | |
| | AATGCATATAGTTATGTGATACATAATGAATGTACAAATGAGA | 1194 |
| | TCCTTACCCCTAAATATAAAAAGATTCCATAGAACATAAATCT | |
| | CCAGAAAAAACATCGCCGAAGGGCATTAATGAGTT | |
| | AATCTTTTTATATTTAG | 1195 |
| | CTAAATATAAAAAGATT | 1196 |
| Cystic fibrosis | AACTCATTAATGCCCTTCGGCGATGTTTTTTCTGGAGATTTAT | 1197 |
| Leu88Term | GTTCTATGGAATCTTTTTATATTTAGGGGTAAGGATCTCATTT | |
| TTA to TAA | GTACATTCATTATGTATCACATAACTATATGCATT | |
| | AATGCATATAGTTATGTGATACATAATGAATGTACAAATGAGA | 1198 |
| | TCCTTACCCCTAAATATAAAAAGATTCCATAGAACATAAATCT | |
| | CCAGAAAAAACATCGCCGAAGGGCATTAATGAGTT | |
| | AATCTTTTTATATTTAG | 1199 |
| | CTAAATATAAAAAGATT | 1200 |
| Cystic fibrosis | AATGCCCTTCGGCGATGTTTTTTCTGGAGATTTATGTTCTATG | 1201 |
| Gly91Ar | GAATCTTTTTATATTTAGGGGTAAGGATCTCATTTGTACATTC | |
| | ATTATGTATCACATAACTATATGCATTTTTGTGAT | |
| | ATCACAAAAATGCATATAGTTATGTGATACATAATGAATGTAC | 1202 |
| | AAATGAGATCCTTACCCCTAAATATAAAAAGATTCCATAGAAC | |
| | ATAAATCTCCAGAAAAAACATCGCCGAAGGGCATT | |
| | TATATTTAGGGGTAAGG | 1203 |
| | CCTTACCCCTAAATATA | 1204 |
| Cystic fibrosis | AATAAATGAAATTTAATTTCTCTGTTTTTCCCCTTTTGTAGGAA | 1205 |
| Gln98A | GTCACCAAAGCAGTACAGCCTCTCTTACTGGGAAGAATCATA | |
| | GCTTCCTATGACCCGGATAACAAGGAGGAACGCTC | |
| | GAGCGTTCCTCCTTGTTATCCGGGTCATAGGAAGCTATGATT | 1206 |
| | CTTCCCAGTAAGAGAGGCTGTACTGCTTTGGTGACTTCCTAC | |
| | AAAAGGGGAAAAACAGAGAAATTAAATTTCATTTATT | |
| | AGCAGTACAGCCTCTCT | 1207 |
| | AGAGAGGCTGTACTGCT | 1208 |
| Cystic fibrosis | AAATAAATGAAATTTAATTTCTCTGTTTTTCCCCTTTTGTAGGA | 1209 |
| Gln98Term | AGTCACCAAAGCAGTACAGCCTCTCTTACTGGGAAGAATCAT | |
| CAG-TAG | AGCTTCCTATGACCCGGATAACAAGGAGGAACGCT | |
| | AGCGTTCCTCCTTGTTATCCGGGTCATAGGAAGCTATGATTC | 1210 |
| | TTCCCAGTAAGAGAGGCTGTACTGCTTTGGTGACTTCCTACA | |
| | AAAGGGGAAAAACAGAGAAATTAAATTTCATTTATTT | |
| | AAGCAGTACAGCCTCTC | 1211 |
| | GAGAGGCTGTACTGCTT | 1212 |
| Cystic fibrosis | CCCTTTTGTAGGAAGTCACCAAAGCAGTACAGCCTCTCTTAC | 1213 |
| Ser108Ph | TGGGAAGAATCATAGCTTCCTATGACCCGGATAACAAGGAGG | |
| | AACGCTCTATCGCGATTTATCTAGGCATAGGCTTATG | |
| | CATAAGCCTATGCCTAGATAAATCGCGATAGAGCGTTCCTCC | 1214 |
| | TTGTTATCCGGGTCATAGGAAGCTATGATTCTTCCCAGTAAG | |
| | AGAGGCTGTACTGCTTTGGTGACTTCCTACAAAAGGG | |
| | CATAGCTTCCTATGACC | 1215 |
| | GGTCATAGGAAGCTATG | 1216 |
| Cystic fibrosis | TTTTGTAGGAAGTCACCAAAGCAGTACAGCCTCTCTTACTGG | 1217 |
| Tyr109Cys | GAAGAATCATAGCTTCCTATGACCCGGATAACAAGGAGGAAC | |
| TAT to TGT | GCTCTATCGCGATTTATCTAGGCATAGGCTTATGCCT | |
| | AGGCATAAGCCTATGCCTAGATAAATCGCGATAGAGCGTTCC | 1218 |
| | TCCTTGTTATCCGGGTCATAGGAAGCTATGATTCTTCCCAGT | |
| | AAGAGAGGCTGTACTGCTTTGGTGACTTCCTACAAAA | |
| | AGCTTCCTATGACCCGG | 1219 |
| | CCGGGTCATAGGAAGCT | 1220 |
| Cystic fibrosis | TTGTAGGAAGTCACCAAAGCAGTACAGCCTCTCTTACTGGGA | 1221 |
| Asp110Hi | AGAATCATAGCTTCCTATGACCCGGATAACAAGGAGGAACGC | |
| | TCTATCGCGATTTATCTAGGCATAGGCTTATGCCTTC | |
| | GAAGGCATAAGCCTATGCCTAGATAAATCGCGATAGAGCGTT | 1222 |
| | CCTCCTTGTTATCCGGGTCATAGGAAGCTATGATTCTTCCCA | |
| | GTAAGAGAGGCTGTACTGCTTTGGTGACTTCCTACAA | |
| | CTTCCTATGACCCGGAT | 1223 |
| | ATCCGGGTCATAGGAAG | 1224 |
| Congenital absence | AGGAAGTCACCAAAGCAGTACAGCCTCTCTTACTGGGAAGAA | 1225 |
| of vas deferens | TCATAGCTTCCTATGACCCGGATAACAAGGAGGAACGCTCTA | |
| Pro111Leu | TCGCGATTTATCTAGGCATAGGCTTATGCCTTCTCTT | |
| CCG to CTG | | |
| | AAGAGAAGGCATAAGCCTATGCCTAGATAAATCGCGATAGAG | 1226 |
| | CGTTCCTCCTTGTTATCCGGGTCATAGGAAGCTATGATTCTT | |
| | CCCAGTAAGAGAGGCTGTACTGCTTTGGTGACTTCCT | |
| | CTATGACCCGGATAACA | 1227 |
| | TGTTATCCGGGTCATAG | 1228 |
| Cystic fibrosis | GTACAGCCTCTCTTACTGGGAAGAATCATAGCTTCCTATGAC | 1229 |
| A | CCGGATAACAAGGAGGAACGCTCTATCGCGATTTATCTAGGC | |
| | ATAGGCTTATGCCTTCTCTTTATTGTGAGGACACTGC | |
| | GCAGTGTCCTCACAATAAAGAGAAGGCATAAGCCTATGCCTA | 1230 |
| | GATAAATCGCGATAGAGCGTTCCTCCTTGTTATCCGGGTCAT | |
| | AGGAAGCTATGATTCTTCCCAGTAAGAGAGGCTGTAC | |
| | AGGAGGAACGCTCTATC | 1231 |
| | GATAGAGCGTTCCTCCT | 1232 |
| Cystic fibrosis | TACAGCCTCTCTTACTGGGAAGAATCATAGCTTCCTATGACC | 1233 |
| Arg117H | CGGATAACAAGGAGGAACGCTCTATCGCGATTTATCTAGGCA | |
| | TAGGCTTATGCCTTCTCTTTATTGTGAGGACACTGCT | |
| | AGCAGTGTCCTCACAATAAAGAGAAGGCATAAGCCTATGCCT | 1234 |
| | AGATAAATCGCGATAGAGCGTTCCTCCTTGTTATCCGGGTCA | |
| | TAGGAAGCTATGATTCTTCCCAGTAAGAGAGGCTGTA | |
| | GGAGGAACGCTCTATCG | 1235 |
| | CGATAGAGCGTTCCTCC | 1236 |
| Cystic fibrosis | TACAGCCTCTCTTACTGGGAAGAATCATAGCTTCCTATGACC | 1237 |
| Arg117L | CGGATAACAAGGAGGAACGCTCTATCGCGATTTATCTAGGCA | |
| | TAGGCTTATGCCTTCTCTTTATTGTGAGGACACTGCT | |
| | AGCAGTGTCCTCACAATAAAGAGAAGGCATAAGCCTATGCCT | 1238 |
| | AGATAAATCGCGATAGAGCGTTCCTCCTTGTTATCCGGGTCA | |
| | TAGGAAGCTATGATTCTTCCCAGTAAGAGAGGCTGTA | |
| | GGAGGAACGCTCTATCG | 1239 |
| | CGATAGAGCGTTCCTCC | 1240 |
| Cystic fibrosis | TACAGCCTCTCTTACTGGGAAGAATCATAGCTTCCTATGACC | 1241 |
| Arg117P | CGGATAACAAGGAGGAACGCTCTATCGCGATTTATCTAGGCA | |
| | TAGGCTTATGCCTTCTCTTTATTGTGAGGACACTGCT | |
| | AGCAGTGTCCTCACAATAAAGAGAAGGCATAAGCCTATGCCT | 1242 |
| | AGATAAATCGCGATAGAGCGTTCCTCCTTGTTATCCGGGTCA | |
| | TAGGAAGCTATGATTCTTCCCAGTAAGAGAGGCTGTA | |
| | GGAGGAACGCTCTATCG | 1243 |
| | CGATAGAGCGTTCCTCC | 1244 |
| Cystic fibrosis | CTCTTACTGGGAAGAATCATAGCTTCCTATGACCCGGATAAC | 1245 |
| | AAGGAGGAACGCTCTATCGCGATTTATCTAGGCATAGGCTTA | |
| | TGCCTTCTCTTTATTGTGAGGACACTGCTCCTACACC | |
| | GGTGTAGGAGCAGTGTCCTCACAATAAAGAGAAGGCATAAG | 1246 |
| | CCTATGCCTAGATAAATCGCGATAGAGCGTTCCTCCTTGTTA | |
| | TCCGGGTCATAGGAAGCTATGATTCTTCCCAGTAAGAG | |
| | GCTCTATCGCGATTTAT | 1247 |
| | ATAAATCGCGATAGAGC | 1248 |
| Cystic fibrosis | GGGAAGAATCATAGCTTCCTATGACCCGGATAACAAGGAGG | 1249 |
| Tyr122Te | AACGCTCTATCGCGATTTATCTAGGCATAGGCTTATGCCTTCT | |
| | CTTTATTGTGAGGACACTGCTCCTACACCCAGCCATT | |
| | AATGGCTGGGTGTAGGAGCAGTGTCCTCACAATAAAGAGAA | 1250 |
| | GGCATAAGCCTATGCCTAGATAAATCGCGATAGAGCGTTCCT | |
| | CCTTGTTATCCGGGTCATAGGAAGCTATGATTCTTCCC | |
| | GCGATTTATCTAGGCAT | 1251 |
| | ATGCCTAGATAAATCGC | 1252 |
| Cystic fibrosis | TAGCTTCCTATGACCCGGATAACAAGGAGGAACGCTCTATCG | 1253 |
| | CGATTTATCTAGGCATAGGCTTATGCCTTCTCTTTATTGTGAG | |
| | GACACTGCTCCTACACCCAGCCATTTTTGGCCTTCA | |
| | TGAAGGCCAAAAATGGCTGGGTGTAGGAGCAGTGTCCTCAC | 1254 |
| | AATAAAGAGAAGGCATAAGCCTATGCCTAGATAAATCGCGAT | |
| | AGAGCGTTCCTCCTTGTTATCCGGGTCATAGGAAGCTA | |
| | AGGCATAGGCTTATGCC | 1255 |
| | GGCATAAGCCTATGCCT | 1256 |
| Cystic fibrosis | TCGCGATTTATCTAGGCATAGGCTTATGCCTTCTCTTTATTGT | 1257 |
| H | GAGGACACTGCTCCTACACCCAGCCATTTTTGGCCTTCATCA | |
| | CATTGGAATGCAGATGAGAATAGCTATGTTTAGTTT | |
| | AAACTAAACATAGCTATTCTCATCTGCATTCCAATGTGATGAA | 1258 |
| | GGCCAAAAATGGCTGGGTGTAGGAGCAGTGTCCTCACAATA | |
| | AAGAGAAGGCATAAGCCTATGCCTAGATAAATCGCGA | |
| | GCTCCTACACCCAGCCA | 1259 |
| | TGGCTGGGTGTAGGAGC | 1260 |
| Cystic fibrosis | TTTATCTAGGCATAGGCTTATGCCTTCTCTTTATTGTGAGGAC | 1261 |
| Ala141A | ACTGCTCCTACACCCAGCCATTTTTGGCCTTCATCACATTGG | |
| | AATGCAGATGAGAATAGCTATGTTTAGTTTGATTTA | |
| | TAAATCAAACTAAACATAGCTATTCTCATCTGCATTCCAATGT | 1262 |
| | GATGAAGGCCAAAAATGGCTGGGTGTAGGAGCAGTGTCCTC | |
| | ACAATAAAGAGAAGGCATAAGCCTATGCCTAGATAAA | |
| | ACACCCAGCCATTTTTG | 1263 |
| | CAAAAATGGCTGGGTGT | 1264 |
| Cystic fibrosis | GCCTTCTCTTTATTGTGAGGACACTGCTCCTACACCCAGCCA | 1265 |
| Ile148T | TTTTTGGCCTTCATCACATTGGAATGCAGATGAGAATAGCTAT | |
| | GTTTAGTTTGATTTATAAGAAGGTAATACTTCCTTG | |
| | CAAGGAAGTATTACCTTCTTATAAATCAAACTAAACATAGCTA | 1266 |
| | TTCTCATCTGCATTCCAATGTGATGAAGGCCAAAAATGGCTG | |
| | GGTGTAGGAGCAGTGTCCTCACAATAAAGAGAAGGC | |
| | TCATCACATTGGAATGC | 1267 |
| | GCATTCCAATGTGATGA | 1268 |
| Cystic fibrosis | CTTCTCTTTATTGTGAGGACACTGCTCCTACACCCAGCCATTT | 1269 |
| Gly149Ar | TTGGCCTTCATCACATTGGAATGCAGATGAGAATAGCTATGT | |
| | TTAGTTTGATTTATAAGAAGGTAATACTTCCTTGCA | |
| | TGCAAGGAAGTATTACCTTCTTATAAATCAAACTAAACATAGC | 1270 |
| | TATTCTCATCTGCATTCCAATGTGATGAAGGCCAAAAATGGCT | |
| | GGGTGTAGGAGCAGTGTCCTCACAATAAAGAGAAG | |
| | ATCACATTGGAATGCAG | 1271 |
| | CTGCATTCCAATGTGAT | 1272 |
| Cystic fibrosis | TTTATTGTGAGGACACTGCTCCTACACCCAGCCATTTTTGGC | 1273 |
| GI | CTTCATCACATTGGAATGCAGATGAGAATAGCTATGTTTAGTT | |
| | TGATTTATAAGAAGGTAATACTTCCTTGCACAGGCC | |
| | GGCCTGTGCAAGGAAGTATTACCTTCTTATAAATCAAACTAAA | 1274 |
| | CATAGCTATTCTCATCTGCATTCCAATGTGATGAAGGCCAAAA | |
| | ATGGCTGGGTGTAGGAGCAGTGTCCTCACAATAAA | |
| | TTGGAATGCAGATGAGA | 1275 |
| | TCTCATCTGCATTCCAA | 1276 |
| Cystic fibrosis | AATATATTTGTATTTTGTTTGTTGAAATTATCTAACTTTCCATTT | 1277 |
| | TTCTTTTAGACTTTAAAGCTGTCAAGCCGTGTTCTAGATAAAA | |
| | TAAGTATTGGACAACTTGTTAGTCTCCTTTCCA | |
| | TGGAAAGGAGACTAACAAGTTGTCCAATACTTATTTTATCTAG | 1278 |
| | AACACGGCTTGACAGCTTTAAAGTCTAAAAGAAAAATGGAAA | |
| | GTTAGATAATTTCAACAAACAAAATACAAATATATT | |
| | AGACTTTAAAGCTGTCA | 1279 |
| | TGACAGCTTTAAAGTCT | 1280 |
| Cystic fibrosis | TTATCTAACTTTCCATTTTTCTTTTAGACTTTAAAGCTGTCAAG | 1281 |
| Ile175Val | CCGTGTTCTAGATAAAATAAGTATTGGACAACTTGTTAGTCTC | |
| ATA-GTA | CTTTCCAACAACCTGAACAAATTTGATGAAGTAT | |
| | ATACTTCATCAAATTTGTTCAGGTTGTTGGAAAGGAGACTAAC | 1282 |
| | AAGTTGTCCAATACTTATTTTATCTAGAACACGGCTTGACAGC | |
| | TTTAAAGTCTAAAAGAAAAATGGAAAGTTAGATAA | |
| | TAGATAAAATAAGTATT | 1283 |
| | AATACTTATTTTATCTA | 1284 |
| Cystic fibrosis | TTTCCATTTTTCTTTTAGACTTTAAAGCTGTCAAGCCGTGTTCT | 1285 |
| G | AGATAAAATAAGTATTGGACAACTTGTTAGTCTCCTTTCCAAC | |
| | AACCTGAACAAATTTGATGAAGTATGTACCTATT | |
| | AATAGGTACATACTTCATCAAATTTGTTCAGGTTGTTGGAAAG | 1286 |
| | GAGACTAACAAGTTGTCCAATACTTATTTTATCTAGAACACGG | |
| | CTTGACAGCTTTAAAGTCTAAAAGAAAAATGGAAA | |
| | TAAGTATTGGACAACTT | 1287 |
| | AAGTTGTCCAATACTTA | 1288 |
| Cystic fibrosis | AAGATACAATGACACCTGTTTTTGCTGTGCTTTTATTTTCCAG | 1289 |
| His199G | GGACTTGCATTGGCACATTTCGTGTGGATCGCTCCTTTGCAA | |
| | GTGGCACTCCTCATGGGGCTAATCTGGGAGTTGTTA | |
| | TAACAACTCCCAGATTAGCCCCATGAGGAGTGCCACTTGCAA | 1290 |
| | AGGAGCGATCCACACGAAATGTGCCAATGCAAGTCCCTGGA | |
| | AAATAAAAGCACAGCAAAAACAGGTGTCATTGTATCTT | |
| | TTGGCACATTTCGTGTG | 1291 |
| | CACACGAAATGTGCCAA | 1292 |
| Cystic fibrosis | GGAAGATACAATGACACCTGTTTTTGCTGTGCTTTTATTTTCC | 1293 |
| His199T | AGGGACTTGCATTGGCACATTTCGTGTGGATCGCTCCTTTGC | |
| | AAGTGGCACTCCTCATGGGGCTAATCTGGGAGTTGT | |
| | ACAACTCCCAGATTAGCCCCATGAGGAGTGCCACTTGCAAAG | 1294 |
| | GAGCGATCCACACGAAATGTGCCAATGCAAGTCCCTGGAAA | |
| | ATAAAAGCACAGCAAAAACAGGTGTCATTGTATCTTCC | |
| | CATTGGCACATTTCGTG | 1295 |
| | CACGAAATGTGCCAATG | 1296 |
| Cystic fibrosis | TGTTTTTGCTGTGCTTTTATTTTCCAGGGACTTGCATTGGCAC | 1297 |
| P | ATTTCGTGTGGATCGCTCCTTTGCAAGTGGCACTCCTCATGG | |
| | GGCTAATCTGGGAGTTGTTACAGGCGTCTGCCTTCT | |
| | AGAAGGCAGACGCCTGTAACAACTCCCAGATTAGCCCCATG | 1298 |
| | AGGAGTGCCACTTGCAAAGGAGCGATCCACACGAAATGTGC | |
| | CAATGCAAGTCCCTGGAAAATAAAAGCACAGCAAAAACA | |
| | GGATCGCTCCTTTGCAA | 1299 |
| | TTGCAAAGGAGCGATCC | 1300 |
| Cystic fibrosis | TTTGCTGTGCTTTTATTTTCCAGGGACTTGCATTGGCACATTT | 1301 |
| L | CGTGTGGATCGCTCCTTTGCAAGTGGCACTCCTCATGGGGC | |
| | TAATCTGGGAGTTGTTACAGGCGTCTGCCTTCTGTGG | |
| | CCACAGAAGGCAGACGCCTGTAACAACTCCCAGATTAGCCC | 1302 |
| | CATGAGGAGTGCCACTTGCAAAGGAGCGATCCACACGAAAT | |
| | GTGCCAATGCAAGTCCCTGGAAAATAAAAGCACAGCAAA | |
| | CGCTCCTTTGCAAGTGG | 1303 |
| | CCACTTGCAAAGGAGCG | 1304 |
| Cystic fibrosis | TTCGTGTGGATCGCTCCTTTGCAAGTGGCACTCCTCATGGGG | 1305 |
| Gln220Te | CTAATCTGGGAGTTGTTACAGGCGTCTGCCTTCTGTGGACTT | |
| | GGTTTCCTGATAGTCCTTGCCCTTTTTCAGGCTGGGC | |
| | GCCCAGCCTGAAAAAGGGCAAGGACTATCAGGAAACCAAGT | 1306 |
| | CCACAGAAGGCAGACGCCTGTAACAACTCCCAGATTAGCCC | |
| | CATGAGGAGTGCCACTTGCAAAGGAGCGATCCACACGAA | |
| | AGTTGTTACAGGCGTCT | 1307 |
| | AGACGCCTGTAACAACT | 1308 |
| Cystic f | CCTTTGCAAGTGGCACTCCTCATGGGGCTAATCTGGGAGTTG | 1309 |
| | TTACAGGCGTCTGCCTTCTGTGGACTTGGTTTCCTGATAGTC | |
| | CTTGCCCTTTTTCAGGCTGGGCTAGGGAGAATGATGA | |
| | TCATCATTCTCCCTAGCCCAGCCTGAAAAAGGGCAAGGACTA | 1310 |
| | TCAGGAAACCAAGTCCACAGAAGGCAGACGCCTGTAACAAC | |
| | TCCCAGATTAGCCCCATGAGGAGTGCCACTTGCAAAGG | |
| | CTGCCTTCTGTGGACTT | 1311 |
| | AAGTCCACAGAAGGCAG | 1312 |
| Cystic fibrosis | TGGGGCTAATCTGGGAGTTGTTACAGGCGTCTGCCTTCTGTG | 1313 |
| V | GACTTGGTTTCCTGATAGTCCTTGCCCTTTTTCAGGCTGGGC | |
| | TAGGGAGAATGATGATGAAGTACAGGTAGCAACCTAT | |
| | ATAGGTTGCTACCTGTACTTCATCATCATTCTCCCTAGCCCAG | 1314 |
| | CCTGAAAAAGGGCAAGGACTATCAGGAAACCAAGTCCACAG | |
| | AAGGCAGACGCCTGTAACAACTCCCAGATTAGCCCCA | |
| | CCTGATAGTCCTTGCCC | 1315 |
| | GGGCAAGGACTATCAGG | 1316 |
| Cystic fibrosis | GTTACAGGCGTCTGCCTTCTGTGGACTTGGTTTCCTGATAGT | 1317 |
| Gly239A | CCTTGCCCTTTTTCAGGCTGGGCTAGGGAGAATGATGATGAA | |
| | GTACAGGTAGCAACCTATTTTCATAACTTGAAAGTTT | |
| | AAACTTTCAAGTTATGAAAATAGGTTGCTACCTGTACTTCATC | 1318 |
| | ATCATTCTCCCTAGCCCAGCCTGAAAAAGGGCAAGGACTATC | |
| | AGGAAACCAAGTCCACAGAAGGCAGACGCCTGTAAC | |
| | TTTCAGGCTGGGCTAGG | 1319 |
| | CCTAGCCCAGCCTGAAA | 1320 |

### EXAMPLE 11

### Cyclin-Dependent Kinase Inhibitor 2A - CDKN2A

The human CDKN2A gene was also designated MTS-1 for multiple tumor suppressor-1 and has been implicated in multiple cancers including, for example, malignant melanoma. Malignant melanoma is a cutaneous neoplasm of melanocytes. Melanomas generally have features of asymmetry, irregular border, variegated color, and diameter greater than 6 mm. The precise cause of melanoma is unknown, but sunlight and heredity are risk factors. Melanoma has been increasing during the past few decades.

The CDKN2A gene has been found to be homozygously deleted at high frequency in cell lines derived from tumors of lung, breast, brain, bone, skin, bladder, kidney, ovary, and lymphocyte. Melanoma cell lines carried at least one copy of CDKN2A in combination with a deleted allele. Melanoma cell lines that carried at least 1 copy of CDKN2A frequently showed nonsense, missense, or frameshift mutations in the gene. Thus, CDKN2A may rival p53 (see Example 6) in the universality of its involvement in tumorigenesis. The attached table discloses the correcting oligonucleotide base sequences for the CDKN2A oligonucleotides of the invention.

TABLE 13
CDKN2A Mutations and Genome-Correcting Oligos

| Clinical Phenotype & | | SEQ ID |
|---|---|---|
| Mutation | Correcting Oligos | NO: |
| Melanoma | GGGCGGCGGGGAGCAGCATGGAGCCGGCGGCGGGGAGCAG | 1321 |
| Trp15Term | CATGGAGCCTTCGGCTGACTGGCTGGCCACGGCCGCGGCCC | |
| TGG-TAG | GGGGTCGGGTAGAGGAGGTGCGGGCGCTGCTGGAGGCGGG | |
| | CCCGCCTCCAGCAGCGCCCGCACCTCCTCTACCCGACCCCG | 1322 |
| | GGCCGCGGCCGTGGCCAGCCAGTCAGCCGAAGGCTCCATGC | |
| | TGCTCCCCGCCGCCGGCTCCATGCTGCTCCCCGCCGCCC | |
| | GGCTGACTGGCTGGCCA | 1323 |
| | TGGCCAGCCAGTCAGCC | 1324 |
| Melanoma | CGGCGGGGAGCAGCATGGAGCCGGCGGCGGGGAGCAGCAT | 1325 |
| Leu16Pro | GGAGCCTTCGGCTGACTGGCTGGCCACGGCCGCGGCCCGGG | |
| CTG-CCG | GTCGGGTAGAGGAGGTGCGGGCGCTGCTGGAGGCGGGGGC | |
| | GCCCCCGCCTCCAGCAGCGCCCGCACCTCCTCTACCCGACC | 1326 |
| | CCGGGCCGCGGCCGTGGCCAGCCAGTCAGCCGAAGGCTCCA | |
| | TGCTGCTCCCCGCCGCCGGCTCCATGCTGCTCCCCGCCG | |
| | TGACTGGCTGGCCACGG | 1327 |
| | CCGTGGCCAGCCAGTCA | 1328 |
| Melanoma | CGGCGGCGGGGAGCAGCATGGAGCCTTCGGCTGACTGGCTG | 1329 |
| Gly23Asp | GCCACGGCCGCGGCCCGGGGTCGGGTAGAGGAGGTGCGGG | |
| GGT-GAT | CGCTGCTGGAGGCGGGGGCGCTGCCCAACGCACCGAATAG | |
| | CTATTCGGTGCGTTGGGCAGCGCCCCCGCCTCCAGCAGCGC | 1330 |
| | CCGCACCTCCTCTACCCGACCCCGGGCCGCGGCCGTGGCCA | |
| | GCCAGTCAGCCGAAGGCTCCATGCTGCTCCCCGCCGCCG | |
| | GGCCCGGGGTCGGGTAG | 1331 |
| | CTACCCGACCCCGGGCC | 1332 |
| Melanoma | CGGCGGGGAGCAGCATGGAGCCTTCGGCTGACTGGCTGGCC | 1333 |
| Arg24Pro | ACGGCCGCGGCCCGGGGTCGGGTAGAGGAGGTGCGGGCGC | |
| CGG-CCG | TGCTGGAGGCGGGGGCGCTGCCCAACGCACCGAATAGTTA | |
| | TAACTATTCGGTGCGTTGGGCAGCGCCCCCGCCTCCAGCAGC | 1334 |
| | GCCCGCACCTCCTCTACCCGACCCCGGGCCGCGGCCGTGGC | |
| | CAGCCAGTCAGCCGAAGGCTCCATGCTGCTCCCCGCCG | |
| | CCGGGGTCGGGTAGAGG | 1335 |
| | CCTCTACCCGACCCCGG | 1336 |
| Melanoma | CGGCTGACTGGCTGGCCACGGCCGCGGCCCGGGGTCGGGT | 1337 |
| Leu32Pro | AGAGGAGGTGCGGGCGCTGCTGGAGGCGGGGGCGCTGCCC | |
| CTG-CCG | AACGCACCGAATAGTTACGGTCGGAGGCCGATCCAGGTGGG | |
| | CCCACCTGGATCGGCCTCCGACCGTAACTATTCGGTGCGTTG | 1338 |
| | GGCAGCGCCCCCGCCTCCAGCAGCGCCCGCACCTCCTCTAC | |
| | CCGACCCCGGGCCGCGGCCGTGGCCAGCCAGTCAGCCG | |
| | GGCGCTGCTGGAGGCGG | 1339 |
| | CCGCCTCCAGCAGCGCC | 1340 |
| Melanoma | GGCTGGCCACGGCCGCGGCCCGGGGTCGGGTAGAGGAGGT | 1341 |
| Gly35Ala | GCGGGCGCTGCTGGAGGCGGGGGCGCTGCCCAACGCACCG | |
| GGG-GCG | AATAGTTACGGTCGGAGGCCGATCCAGGTGGGTAGAGGGTC | |
| | GACCCTCTACCCACCTGGATCGGCCTCCGACCGTAACTATTC | 1342 |
| | GGTGCGTTGGGCAGCGCCCCCGCCTCCAGCAGCGCCCGCAC | |
| | CTCCTCTACCCGACCCCGGGCCGCGGCCGTGGCCAGCC | |
| | GGAGGCGGGGGCGCTGC | 1343 |
| | GCAGCGCCCCCGCCTCC | 1344 |
| Melanoma | GGTAGAGGAGGTGCGGGCGCTGCTGGAGGCGGGGGCGCTG | 1345 |
| Tyr44Term | CCCAACGCACCGAATAGTTACGGTCGGAGGCCGATCCAGGTG | |
| TACg-TAA | GGTAGAGGGTCTGCAGCGGGAGCAGGGGATGGCGGGCGA | |
| | TCGCCCGCCATCCCCTGCTCCCGCTGCAGACCCTCTACCCAC | 1346 |
| | CTGGATCGGCCTCCGACCGTAACTATTCGGTGCGTTGGGCAG | |
| | CGCCCCCGCCTCCAGCAGCGCCCGCACCTCCTCTACC | |
| | AATAGTTACGGTCGGAG | 1347 |
| | CTCCGACCGTAACTATT | 1348 |
| Melanoma | TCTCCCATACCTGCCCCCACCCTGGCTCTGACCACTCTGCTCT | 1349 |
| Met53Ile | CTCTGGCAGGTCATGATGATGGGCAGCGCCCGCGTGGCGGA | |
| ATGa-ATC | GCTGCTGCTGCTCCACGGCGCGGAGCCCAACTGCGCA | |
| | TGCGCAGTTGGGCTCCGCGCCGTGGAGCAGCAGCAGCTCCG | 1350 |
| | CCACGCGGGCGCTGCCCATCATCATGACCTGCCAGAGAGAG | |
| | CAGAGTGGTCAGAGCCAGGGTGGGGGCAGGTATGGGAGA | |
| | GTCATGATGATGGGCAG | 1351 |
| | CTGCCCATCATCATGAC | 1352 |
| Melanoma | CCCATACCTGCCCCCACCCTGGCTCTGACCACTCTGCTCTCTC | 1353 |
| Met54Ile | TGGCAGGTCATGATGATGGGCAGCGCCCGCGTGGCGGAGCT | |
| ATGg-ATT | GCTGCTGCTCCACGGCGCGGAGCCCAACTGCGCAGAC | |
| | GTCTGCGCAGTTGGGCTCCGCGCCGTGGAGCAGCAGCAGCT | 1354 |
| | CCGCCACGCGGGCGCTGCCCATCATCATGACCTGCCAGAGA | |
| | GAGCAGAGTGGTCAGAGCCAGGGTGGGGGCAGGTATGGG | |
| | ATGATGATGGGCAGCGC | 1355 |
| | GCGCTGCCCATCATCAT | 1356 |
| Melanoma | GCCGGCCCCCACCCTGGCTCTGACCATTCTGTTCTCTCTGGC | 1357 |
| Ser56Ile | AGGTCATGATGATGGGCAGCGCCCGAGTGGCGGAGCTGCTG | |
| AGC-ATC | CTGCTCCACGGCGCGGAGCCCAACTGCGCCGACCCCGC | |
| | GCGGGGTCGGCGCAGTTGGGCTCCGCGCCGTGGAGCAGCA | 1358 |
| | GCAGCTCCGCCACTCGGGCGCTGCCCATCATCATGACCTGCC | |
| | AGAGAGAACAGAATGGTCAGAGCCAGGGTGGGGGCCGGC | |
| | GATGGGCAGCGCCCGAG | 1359 |
| | CTCGGGCGCTGCCCATC | 1360 |
| Melanoma | GGCCCCCACCCTGGCTCTGACCATTCTGTTCTCTCTGGCAGG | 1361 |
| Ala57Val | TCATGATGATGGGCAGCGCCCGAGTGGCGGAGCTGCTGCTG | |
| GCC-GTC | CTCCACGGCGCGGAGCCCAACTGCGCCGACCCCGCCAC | |
| | GTGGCGGGGTCGGCGCAGTTGGGCTCCGCGCCGTGGAGCA | 1362 |
| | GCAGCAGCTCCGCCACTCGGGCGCTGCCCATCATCATGACCT | |
| | GCCAGAGAGAACAGAATGGTCAGAGCCAGGGTGGGGGCC | |
| | GGGCAGCGCCCGAGTGG | 1363 |
| | CCACTCGGGCGCTGCCC | 1364 |
| Melanoma | CCCCCACCCTGGCTCTGACCATTCTGTTCTCTCTGGCAGGTCA | 1365 |
| Arg58Term | TGATGATGGGCAGCGCCCGAGTGGCGGAGCTGCTGCTGCTC | |
| cCGA-TGA | CACGGCGCGGAGCCCAACTGCGCCGACCCCGCCACTC | |
| | GAGTGGCGGGGTCGGCGCAGTTGGGCTCCGCGCCGTGGAG | 1366 |
| | CAGCAGCAGCTCCGCCACTCGGGCGCTGCCCATCATCATGAC | |
| | CTGCCAGAGAGAACAGAATGGTCAGAGCCAGGGTGGGGG | |
| | GCAGCGCCCGAGTGGCG | 1367 |
| | CGCCACTCGGGCGCTGC | 1368 |
| Melanoma | CACCCTGGCTCTGACCATTCTGTTCTCTCTGGCAGGTCATGAT | 1369 |
| Val59Gly | GATGGGCAGCGCCCGAGTGGCGGAGCTGCTGCTGCTCCACG | |
| GTG-GGG | GCGCGGAGCCCAACTGCGCCGACCCCGCCACTCTCAC | |
| | GTGAGAGTGGCGGGGTCGGCGCAGTTGGGCTCCGCGCCGTG | 1370 |
| | GAGCAGCAGCAGCTCCGCCACTCGGGCGCTGCCCATCATCAT | |
| | GACCTGCCAGAGAGAACAGAATGGTCAGAGCCAGGGTG | |
| | CGCCCGAGTGGCGGAGC | 1371 |
| | GCTCCGCCACTCGGGCG | 1372 |
| Melanoma | TCTGACCACTCTGCTCTCTCTGGCAGGTCATGATGATGGGCA | 1373 |
| Leu62Pro | GCGCCCGCGTGGCGGAGCTGCTGCTGCTCCACGGCGCGGAG | |
| CTG-CCG | CCCAACTGCGCAGACCCTGCCACTCTCACCCGACCGGT | |
| | ACCGGTCGGGTGAGAGTGGCAGGGTCTGCGCAGTTGGGCTC | 1374 |
| | CGCGCCGTGGAGCAGCAGCAGCTCCGCCACGCGGGCGCTGC | |
| | CCATCATCATGACCTGCCAGAGAGAGCAGAGTGGTCAGA | |
| | GGCGGAGCTGCTGCTGC | 1375 |
| | GCAGCAGCAGCTCCGCC | 1376 |
| Melanoma | TCTGGCAGGTCATGATGATGGGCAGCGCCCGCGTGGCGGAG | 1377 |
| Ala68Val | CTGCTGCTGCTCCACGGCGCGGAGCCCAACTGCGCAGACCC | |
| GCG-GTG | TGCCACTCTCACCCGACCGGTGCATGATGCTGCCCGGGA | |
| | TCCCGGGCAGCATCATGCACCGGTCGGGTGAGAGTGGCAGG | 1378 |
| | GTCTGCGCAGTTGGGCTCCGCGCCGTGGAGCAGCAGCAGCT | |
| | CCGCCACGCGGGCGCTGCCCATCATCATGACCTGCCAGA | |
| | CCACGGCGCGGAGCCCA | 1379 |
| | TGGGCTCCGCGCCGTGG | 1380 |
| Melanoma | CATGATGATGGGCAGCGCCCGAGTGGCGGAGCTGCTGCTGC | 1381 |
| Asn71Lys | TCCACGGCGCGGAGCCCAACTGCGCCGACCCCGCCACTCTC | |
| AACt-AAA | ACCCGACCCGTGCACGACGCTGCCCGGGAGGGCTTCCTG | |
| | CAGGAAGCCCTCCCGGGCAGCGTCGTGCACGGGTCGGGTGA | 1382 |
| | GAGTGGCGGGGTCGGCGCAGTTGGGCTCCGCGCCGTGGAG | |
| | CAGCAGCAGCTCCGCCACTCGGGCGCTGCCCATCATCATG | |
| | GAGCCCAACTGCGCCGA | 1383 |
| | TCGGCGCAGTTGGGCTC | 1384 |
| Melanoma | TCATGATGATGGGCAGCGCCCGAGTGGCGGAGCTGCTGCTG | 1385 |
| Asn71Ser | CTCCACGGCGCGGAGCCCAACTGCGCCGACCCCGCCACTCT | |
| AAC-AGC | CACCCGACCCGTGCACGACGCTGCCCGGGAGGGCTTCCT | |
| | AGGAAGCCCTCCCGGGCAGCGTCGTGCACGGGTCGGGTGAG | 1386 |
| | AGTGGCGGGGTCGGCGCAGTTGGGCTCCGCGCCGTGGAGCA | |
| | GCAGCAGCTCCGCCACTCGGGCGCTGCCCATCATCATGA | |
| | GGAGCCCAACTGCGCCG | 1387 |
| | CGGCGCAGTTGGGCTCC | 1388 |
| Melanoma | AGCTGCTGCTGCTCCACGGCGCGGAGCCCAACTGCGCCGAC | 1389 |
| Pro81Leu | CCCGCCACTCTCACCCGACCCGTGCACGACGCTGCCCGGGA | |
| CCC-CTC | GGGCTTCCTGGACACGCTGGTGGTGCTGCACCGGGCCGG | |
| | CCGGCCCGGTGCAGCACCACCAGCGTGTCCAGGAAGCCCTC | 1390 |
| | CCGGGCAGCGTCGTGCACGGGTCGGGTGAGAGTGGCGGGG | |
| | TCGGCGCAGTTGGGCTCCGCGCCGTGGAGCAGCAGCAGCT | |
| | CACCCGACCCGTGCACG | 1391 |
| | CGTGCACGGGTCGGGTG | 1392 |
| Melanoma | CTGCTCCACGGCGCGGAGCCCAACTGCGCCGACCCCGCCAC | 1393 |
| Asp84Tyr | TCTCACCCGACCCGTGCACGACGCTGCCCGGGAGGGCTTCCT | |
| cGAC-TAC | GGACACGCTGGTGGTGCTGCACCGGGCCGGGGCGCGGC | |
| | GCCGCGCCCCGGCCCGGTGCAGCACCACCAGCGTGTCCAGG | 1394 |
| | AAGCCCTCCCGGGCAGCGTCGTGCACGGGTCGGGTGAGAGT | |
| | GGCGGGGTCGGCGCAGTTGGGCTCCGCGCCGTGGAGCAG | |
| | CCGTGCACGACGCTGCC | 1395 |
| | GGCAGCGTCGTGCACGG | 1396 |
| Melanoma | CTCCACGGCGCGGAGCCCAACTGCGCCGACCCCGCCACTCT | 1397 |
| Ala85Thr | CACCCGACCCGTGCACGACGCTGCCCGGGAGGGCTTCCTGG | |
| cGCT-ACT | ACACGCTGGTGGTGCTGCACCGGGCCGGGGCGCGGCTGG | |
| | CCAGCCGCGCCCCGGCCCGGTGCAGCACCACCAGCGTGTCC | 1398 |
| | AGGAAGCCCTCCCGGGCAGCGTCGTGCACGGGTCGGGTGAG | |
| | AGTGGCGGGGTCGGCGCAGTTGGGCTCCGCGCCGTGGAG | |
| | TGCACGACGCTGCCCGG | 1399 |
| | CCGGGCAGCGTCGTGCA | 1400 |
| Melanoma | GCGCGGAGCCCAACTGCGCCGACCCCGCCACTCTCACCCGA | 1401 |
| Arg87Pro | CCCGTGCACGACGCTGCCCGGGAGGGCTTCCTGGACACGCT | |
| CGG-CCG | GGTGGTGCTGCACCGGGCCGGGGCGCGGCTGGACGTGCG | |
| | CGCACGTCCAGCCGCGCCCCGGCCCGGTGCAGCACCACCAG | 1402 |
| | CGTGTCCAGGAAGCCCTCCCGGGCAGCGTCGTGCACGGGTC | |
| | GGGTGAGAGTGGCGGGGTCGGCGCAGTTGGGCTCCGCGC | |
| | CGCTGCCCGGGAGGGCT | 1403 |
| | AGCCCTCCCGGGCAGCG | 1404 |
| Melanoma | GGCGCGGAGCCCAACTGCGCCGACCCCGCCACTCTCACCCG | 1405 |
| Arg87Trp | ACCCGTGCACGACGCTGCCCGGGAGGGCTTCCTGGACACGC | |
| cCGG-TGG | TGGTGGTGCTGCACCGGGCCGGGGCGCGGCTGGACGTGC | |
| | GCACGTCCAGCCGCGCCCCGGCCCGGTGCAGCACCACCAGC | 1406 |
| | GTGTCCAGGAAGCCCTCCCGGGCAGCGTCGTGCACGGGTCG | |
| | GGTGAGAGTGGCGGGGTCGGCGCAGTTGGGCTCCGCGCC | |
| | ACGCTGCCCGGGAGGGC | 1407 |
| | GCCCTCCCGGGCAGCGT | 1408 |
| Melanoma | CTCTCACCCGACCGGTGCATGATGCTGCCCGGGAGGGCTTCC | 1409 |
| Leu97Arg | TGGACACGCTGGTGGTGCTGCACCGGGCCGGGGCGCGGCTG | |
| CTG-CGG | GACGTGCGCGATGCCTGGGGTCGTCTGCCCGTGGACTT | |
| | AAGTCCACGGGCAGACGACCCCAGGCATCGCGCACGTCCAG | 1410 |
| | CCGCGCCCCGGCCCGGTGCAGCACCACCAGCGTGTCCAGGA | |
| | AGCCCTCCCGGGCAGCATCATGCACCGGTCGGGTGAGAG | |
| | GGTGGTGCTGCACCGGG | 1411 |
| | CCCGGTGCAGCACCACC | 1412 |
| Melanoma | CCCGACCGGTGCATGATGCTGCCCGGGAGGGCTTCCTGGAC | 1413 |
| Arg99Pro | ACGCTGGTGGTGCTGCACCGGGCCGGGGCGCGGCTGGACGT | |
| CGG-CCG | GCGCGATGCCTGGGGTCGTCTGCCCGTGGACTTGGCCGA | |
| | TCGGCCAAGTCCACGGGCAGACGACCCCAGGCATCGCGCAC | 1414 |
| | GTCCAGCCGCGCCCCGGCCCGGTGCAGCACCACCAGCGTGT | |
| | CCAGGAAGCCCTCCCGGGCAGCATCATGCACCGGTCGGG | |
| | GCTGCACCGGGCCGGGG | 1415 |
| | CCCCGGCCCGGTGCAGC | 1416 |
| Melanoma | CCGGTGCATGATGCTGCCCGGGAGGGCTTCCTGGACACGCT | 1417 |
| Gly101Trp | GGTGGTGCTGCACCGGGCCGGGGCGCGGCTGGACGTGCGC | |
| cGGG-TGG | GATGCCTGGGGTCGTCTGCCCGTGGACTTGGCCGAGGAGC | |
| | GCTCCTCGGCCAAGTCCACGGGCAGACGACCCCAGGCATCG | 1418 |
| | CGCACGTCCAGCCGCGCCCCGGCCCGGTGCAGCACCACCAG | |
| | CGTGTCCAGGAAGCCCTCCCGGGCAGCATCATGCACCGG | |
| | ACCGGGCCGGGGCGCGG | 1419 |
| | CCGCGCCCCGGCCCGGT | 1420 |
| Melanoma | CGGGAGGGCTTCCTGGACACGCTGGTGGTGCTGCACCGGGC | 1421 |
| Arg107Cys | CGGGGCGCGGCTGGACGTGCGCGATGCCTGGGGTCGTCTGC | |
| gCGC-TGC | CCGTGGACTTGGCCGAGGAGCGGGGCCACCGCGACGTTG | |
| | CAACGTCGCGGTGGCCCCGCTCCTCGGCCAAGTCCACGGGC | 1422 |
| | AGACGACCCCAGGCATCGCGCACGTCCAGCCGCGCCCCGGC | |
| | CCGGTGCAGCACCACCAGCGTGTCCAGGAAGCCCTCCCG | |
| | TGGACGTGCGCGATGCC | 1423 |
| | GGCATCGCGCACGTCCA | 1424 |
| Melanoma | CACCGGGCCGGGGCGCGGCTGGACGTGCGCGATGCCTGGG | 1425 |
| Ala118Thr | GCCGTCTGCCCGTGGACCTGGCTGAGGAGCTGGGCCATCGC | |
| gGCT-ACT | GATGTCGCACGGTACCTGCGCGCGGCTGCGGGGGGCACCA | |
| | TGGTGCCCCCCGCAGCCGCGCGCAGGTACCGTGCGACATCG | 1426 |
| | CGATGGCCCAGCTCCTCAGCCAGGTCCACGGGCAGACGGCC | |
| | CCAGGCATCGCGCACGTCCAGCCGCGCCCCGGCCCGGTG | |
| | TGGACCTGGCTGAGGAG | 1427 |
| | CTCCTCAGCCAGGTCCA | 1428 |
| Melanoma | TGCGCGATGCCTGGGGCCGTCTGCCCGTGGACCTGGCTGAG | 1429 |
| Val126Asp | GAGCTGGGCCATCGCGATGTCGCACGGTACCTGCGCGCGGC | |
| GTC-GAC | TGCGGGGGGCACCAGAGGCAGTAACCATGCCCGCATAGA | |
| | TCTATGCGGGCATGGTTACTGCCTCTGGTGCCCCCCGCAGCC | 1430 |
| | GCGCGCAGGTACCGTGCGACATCGCGATGGCCCAGCTCCTC | |
| | AGCCAGGTCCACGGGCAGACGGCCCCAGGCATCGCGCA | |
| | TCGCGATGTCGCACGGT | 1431 |
| | ACCGTGCGACATCGCGA | 1432 |

### EXAMPLE 12

### Adenomatous Polyposis of the Colon—APC

Adenomatous polyposis of the colon is characterized by adenomatous polyps of the colon and rectum; in extreme cases the bowel is carpeted with a myriad of polyps. This is a viciously premalignant disease with one or more polyps progressing through dysplasia to malignancy in untreated gene carriers with a median age at diagnosis of 40 years.

Mutations in the APC gene are an initiating event for both familial and sporadic colorectal tumorigenesis and many alleles of the APC gene have been identified. Carcinoma may arise at any age from late childhood through the seventh decade with presenting features including, for example, weight loss and inanition, bowel obstruction, or bloody diarrhea. Cases of new mutation still present in these ways but in areas with well organized registers most other gene carriers are detected. The attached table discloses the correcting oligonucleotide base sequences for the APC oligonucleotides of the invention.

TABLE 14
APC Mutations And Genome-Correcting Oligos

| Clinical Phenotype & | | SEQ ID |
|---|---|---|
| Mutation | Correcting Oligos | NO: |
| Adenomatous polyposis | GGATCTGTATCAAGCCGTTCTGGAGAGTGCAGTCCTGTTCCT | 1433 |
| coli | ATGGGTTCATTTCCAAGAAGAGGGTTTGTAAATGGAAGCAGA | |
| Arg121 Term | GAAAGTACTGGATATTTAGAAGAACTTGAGAAAGAGA | |
| AGA-TGA | TCTCTTTCTCAAGTTCTTCTAAATATCCAGTACTTTCTCTGCTT | 1434 |
| | CCATTTACAAACCCTCTTCTTGGAAATGAACCCATAGGAACAG | |
| | GACTGCACTCTCCAGAACGGCTTGATACAGATCC | |
| | TTCCAAGAAGAGGGTTT | 1435 |
| | AAACCCTCTTCTTGGAA | 1436 |
| Adenomatous polyposis | AAAAAAAAAATAGGTCATTGCTTCTTGCTGATCTTGACAAAGA | 1437 |
| coli | AGAAAAGGAAAAAGACTGGTATTACGCTCAACTTCAGAATCTC | |
| Trp157Term | ACTAAAAGAATAGATAGTCTTCCTTTAACTGAAAA | |
| TGG-TAG | TTTTCAGTTAAAGGAAGACTATCTATTCTTTTAGTGAGATTCTG | 1438 |
| | AAGTTGAGCGTAATACCAGTCTTTTTCCTTTTCTTCTTTGTCAA | |
| | GATCAGCAAGAAGCAATGACCTATTTTTTTTTT | |
| | AAAAGACTGGTATTACG | 1439 |
| | CGTAATACCAGTCTTTT | 1440 |
| Adenomatous polyposis | AAATAGGTCATTGCTTCTTGCTGATCTTGACAAAGAAGAAAAG | 1441 |
| coli | GAAAAAGACTGGTATTACGCTCAACTTCAGAATCTCACTAAAA | |
| Tyr159Term | GAATAGATAGTCTTCCTTTAACTGAAAATGTAAGT | |
| TAC-TAG | ACTTACATTTTCAGTTAAAGGAAGACTATCTATTCTTTTAGTGA | 1442 |
| | GATTCTGAAGTTGAGCGTAATACCAGTCTTTTTCCTTTTCTTCT | |
| | TTGTCAAGATCAGCAAGAAGCAATGACCTATTT | |
| | TGGTATTACGCTCAACT | 1443 |
| | AGTTGAGCGTAATACCA | 1444 |
| Adenomatous polyposis | TTGCTTCTTGCTGATCTTGACAAAGAAGAAAAGGAAAAAGACT | 1445 |
| coli | GGTATTACGCTCAACTTCAGAATCTCACTAAAAGAATAGATAG | |
| Gln163Term | TCTTCCTTTAACTGAAAATGTAAGTAACTGGCAGT | |
| CAG-TAG | ACTGCCAGTTACTTACATTTTCAGTTAAAGGAAGACTATCTATT | 1446 |
| | CTTTTAGTGAGATTCTGAAGTTGAGCGTAATACCAGTCTTTTT | |
| | CCTTTTCTTCTTTGTCAAGATCAGCAAGAAGCAA | |
| | CTCAACTTCAGAATCTC | 1447 |
| | GAGATTCTGAAGTTGAG | 1448 |
| Adenomatous polyposis | CTTGACAAAGAAGAAAAGGAAAAAGACTGGTATTACGCTCAAC | 1449 |
| coli | TTCAGAATCTCACTAAAAGAATAGATAGTCTTCCTTTAACTGAA | |
| Arg168Term | AATGTAAGTAACTGGCAGTACAACTTATTTGAAA | |
| AGA-TGA | TTTCAAATAAGTTGTACTGCCAGTTACTTACATTTTCAGTTAAA | 1450 |
| | GGAAGACTATCTATTCTTTTAGTGAGATTCTGAAGTTGAGCGT | |
| | AATACCAGTCTTTTTCCTTTTCTTCTTTGTCAAG | |
| | TCACTAAAAGAATAGAT | 1451 |
| | ATCTATTCTTTTAGTGA | 1452 |
| Adenomatous polyposis | AAGAAAAGGAAAAAGACTGGTATTACGCTCAACTTCAGAATCT | 1453 |
| coli | CACTAAAAGAATAGATAGTCTTCCTTTAACTGAAAATGTAAGTA | |
| Ser171Ile | ACTGGCAGTACAACTTATTTGAAACTTTAATAAC | |
| AGT-ATT | GTTATTAAAGTTTCAAATAAGTTGTACTGCCAGTTACTTACATT | 1454 |
| | TTCAGTTAAAGGAAGACTATCTATTCTTTTAGTGAGATTCTGAA | |
| | GTTGAGCGTAATACCAGTCTTTTTCCTTTTCTT | |
| | AATAGATAGTCTTCCTT | 1455 |
| | AAGGAAGACTATCTATT | 1456 |
| Adenomatous polyposis | GATTAACGTAAATACAAGATATTGATACTTTTTTATTATTTGTG | 1457 |
| coli | GTTTTAGTTTTCCTTACAAACAGATATGACCAGAAGGCAATTG | |
| Gln181Term | GAATATGAAGCAAGGCAAATCAGAGTTGCGATGG | |
| CAA-TAA | CCATCGCAACTCTGATTTGCCTTGCTTCATATTCCAATTGCCT | 1458 |
| | TCTGGTCATATCTGTTTGTAAGGAAAACTAAAACCACAAATAAT | |
| | AAAAAAGTATCAATATCTTGTATTTACGTTAATC | |
| | TTTCCTTACAAACAGAT | 1459 |
| | ATCTGTTTGTAAGGAAA | 1460 |
| Adenomatous polyposis | CTTTTTTATTATTTGTGGTTTTAGTTTTCCTTACAAACAGATATG | 1461 |
| coli | ACCAGAAGGCAATTGGAATATGAAGCAAGGCAAATCAGAGTT | |
| Glu190Term | GCGATGGAAGAACAACTAGGTACCTGCCAGGATA | |
| GAA-TAA | TATCCTGGCAGGTACCTAGTTGTTCTTCCATCGCAACTCTGAT | 1462 |
| | TTGCCTTGCTTCATATTCCAATTGCCTTCTGGTCATATCTGTTT | |
| | GTAAGGAAAACTAAAACCACAAATAATAAAAAAG | |
| | GGCAATTGGAATATGAA | 1463 |
| | TTCATATTCCAATTGCC | 1464 |
| Adenomatous polyposis | CAATTGGAATATGAAGCAAGGCAAATCAGAGTTGCGATGGAA | 1465 |
| coli | GAACAACTAGGTACCTGCCAGGATATGGAAAAACGAGCACAG | |
| Gln208Term | GTAAGTTACTTGTTTCTAAGTGATAAAACAGCGAAGA | |
| CAG-TAG | TCTTCGCTGTTTTATCACTTAGAAACAAGTAACTTACCTGTGCT | 1466 |
| | CGTTTTTCCATATCCTGGCAGGTACCTAGTTGTTCTTCCATCG | |
| | CAACTCTGATTTGCCTTGCTTCATATTCCAATTG | |
| | GTACCTGCCAGGATATG | 1467 |
| | CATATCCTGGCAGGTAC | 1468 |
| Adenomatous polyposis | GCAAGGCAAATCAGAGTTGCGATGGAAGAACAACTAGGTACC | 1469 |
| coli | TGCCAGGATATGGAAAAACGAGCACAGGTAAGTTACTTGTTTC | |
| Arg213Term | TAAGTGATAAAACAGCGAAGAGCTATTAGGAATAAA | |
| CGA-TGA | TTTATTCCTAATAGCTCTTCGCTGTTTTATCACTTAGAAACAAG | 1470 |
| | TAACTTACCTGTGCTCGTTTTTCCATATCCTGGCAGGTACCTA | |
| | GTTGTTCTTCCATCGCAACTCTGATTTGCCTTGC | |
| | TGGAAAAACGAGCACAG | 1471 |
| | CTGTGCTCGTTTTTCCA | 1472 |
| Adenomatous polyposis | GTTTTATTTTAGCGAAGAATAGCCAGAATTCAGCAAATCGAAA | 1473 |
| coli | AGGACATACTTCGTATACGACAGCTTTTACAGTCCCAAGCAAC | |
| Arg232Term | AGAAGCAGAGGTTAGTAAATTGCCTTTCTTGTTTG | |
| CGA-TGA | CAAACAAGAAAGGCAATTTACTAACCTCTGCTTCTGTTGCTTG | 1474 |
| | GGACTGTAAAAGCTGTCGTATACGAAGTATGTCCTTTTCGATT | |
| | TGCTGAATTCTGGCTATTCTTCGCTAAAATAAAAC | |
| | TTCGTATACGACAGCTT | 1475 |
| | AAGCTGTCGTATACGAA | 1476 |
| Adenomatous polyposis | TTATTTTAGCGAAGAATAGCCAGAATTCAGCAAATCGAAAAGG | 1477 |
| coli | ACATACTTCGTATACGACAGCTTTTACAGTCCCAAGCAACAGA | |
| Gln233Term | AGCAGAGGTTAGTAAATTGCCTTTCTTGTTTGTGG | |
| CAG-TAG | CCACAAACAAGAAAGGCAATTTACTAACCTCTGCTTCTGTTGC | 1478 |
| | TTGGGACTGTAAAAGCTGTCGTATACGAAGTATGTCCTTTTCG | |
| | ATTTGCTGAATTCTGGCTATTCTTCGCTAAAATAA | |
| | GTATACGACAGCTTTTA | 1479 |
| | TAAAAGCTGTCGTATAC | 1480 |
| Adenomatous polyposis | AGAAAGCCTACACCATTTTTGCATGTACTGATGTTAACTCCAT | 1481 |
| coli | CTTAACAGAGGTCATCTCAGAACAAGCATGAAACCGGCTCAC | |
| Gln247Term | ATGATGCTGAGCGGCAGAATGAAGGTCAAGGAGTGG | |
| CAG-TAG | CCACTCCTTGACCTTCATTCTGCCGCTCAGCATCATGTGAGC | 1482 |
| | CGGTTTCATGCTTGTTCTGAGATGACCTCTGTTAAGATGGAGT | |
| | TAACATCAGTACATGCAAAAATGGTGTAGGCTTTCT | |
| | GGTCATCTCAGAACAAG | 1483 |
| | CTTGTTCTGAGATGACC | 1484 |
| Adenomatous polyposis | CAGAACAAGCATGAAACCGGCTCACATGATGCTGAGCGGCAG | 1485 |
| coli | AATGAAGGTCAAGGAGTGGGAGAAATCAACATGGCAACTTCT | |
| Gly267Term | GGTAATGGTCAGGTAAATAAATTATTTTATCATATTT | |
| GGA-TGA | AAATATGATAAAATAATTTATTTACCTGACCATTACCAGAAGTT | 1486 |
| | GCCATGTTGATTTCTCCCACTCCTTGACCTTCATTCTGCCGCT | |
| | CAGCATCATGTGAGCCGGTTTCATGCTTGTTCTG | |
| | AAGGAGTGGGAGAAATC | 1487 |
| | GATTTCTCCCACTCCTT | 1488 |
| Adenomatous polyposis | CTTCAAATAACAAAGCATTATGGTTTATGTTGATTTTATTTTTCA | 1489 |
| coli | GTGCCAGCTCCTGTTGAACATCAGATCTGTCCTGCTGTGTGT | |
| Glu443Term | GTTCTAATGAAACTTTCATTTGATGAAGAGCATA | |
| GAA-TAA | TATGCTCTTCATCAAATGAAAGTTTCATTAGAACACACACAGC | 1490 |
| | AGGACAGATCTGATGTTCAACAGGAGCTGGCACTGAAAAATA | |
| | AAATCAACATAAACCATAATGCTTTGTTATTTGAAG | |
| | CTCCTGTTGAACATCAG | 1491 |
| | CTGATGTTCAACAGGAG | 1492 |
| Adenomatous polyposis | CAGTGCCAGCTCCTGTTGAACATCAGATCTGTCCTGCTGTGT | 1493 |
| coli | GTGTTCTAATGAAACTTTCATTTGATGAAGAGCATAGACATGC | |
| SER457TER | AATGAATGAACTAGGTAAGACAAAAATGTTTTTTAA | |
| TCA-TAA | TTAAAAAACATTTTTGTCTTACCTAGTTCATTCATTGCATGTCT | 1494 |
| | ATGCTCTTCATCAAATGAAAGTTTCATTAGAACACACACAGCA | |
| | GGACAGATCTGATGTTCAACAGGAGCTGGCACTG | |
| | GAAACTTTCATTTGATG | 1495 |
| | CATCAAATGAAAGTTTC | 1496 |
| Adenomatous polyposis | AGTTGTTTTATTTTAGATGATTGTCTTTTTCCTCTTGCCCTTTTT | 1497 |
| coli | AAATTAGGGGGACTACAGGCCATTGCAGAATTATTGCAAGTG | |
| Gln473Term | GACTGTGAAATGTACGGGCTTACTAATGACCACT | |
| CAG-TAG | AGTGGTCATTAGTAAGCCCGTACATTTCACAGTCCACTTGCAA | 1498 |
| | TAATTCTGCAATGGCCTGTAGTCCCCCTAATTTAAAAAGGGCA | |
| | AGAGGAAAAAGACAATCATCTAAAATAAAACAACT | |
| | GGGGACTACAGGCCATT | 1499 |
| | AATGGCCTGTAGTCCCC | 1500 |
| Adenomatous polyposis | TTTTAAATTAGGGGGACTACAGGCCATTGCAGAATTATTGCAA | 1501 |
| coli | GTGGACTGTGAAATGTACGGGCTTACTAATGACCACTACAGT | |
| Tyr486Term | ATTACACTAAGACGATATGCTGGAATGGCTTTGACA | |
| TAC-TAG | TGTCAAAGCCATTCCAGCATATCGTCTTAGTGTAATACTGTAG | 1502 |
| | TGGTCATTAGTAAGCCCGTACATTTCACAGTCCACTTGCAATA | |
| | ATTCTGCAATGGCCTGTAGTCCCCCTAATTTAAAA | |
| | GAAATGTACGGGCTTAC | 1503 |
| | GTAAGCCCGTACATTTC | 1504 |
| Adenomatous polyposis | TTGCAAGTGGACTGTGAAATGTATGGGCTTACTAATGACCACT | 1505 |
| coli | ACAGTATTACACTAAGACGATATGCTGGAATGGCTTTGACAAA | |
| Arg499Term | CTTGACTTTTGGAGATGTAGCCAACAAGGTATGTT | |
| CGA-TGA | AACATACCTTGTTGGCTACATCTCCAAAAGTCAAGTTTGTCAA | 1506 |
| | AGCCATTCCAGCATATCGTCTTAGTGTAATACTGTAGTGGTCA | |
| | TTAGTAAGCCCATACATTTCACAGTCCACTTGCAA | |
| | CACTAAGACGATATGCT | 1507 |
| | AGCATATCGTCTTAGTG | 1508 |
| Adenomatous polyposis | AGTGGACTGTGAAATGTATGGGCTTACTAATGACCACTACAGT | 1509 |
| coli | ATTACACTAAGACGATATGCTGGAATGGCTTTGACAAACTTGA | |
| Tyr500Term | CTTTTGGAGATGTAGCCAACAAGGTATGTTTTTAT | |
| TAT-TAG | ATAAAAACATACCTTGTTGGCTACATCTCCAAAAGTCAAGTTT | 1510 |
| | GTCAAAGCCATTCCAGCATATCGTCTTAGTGTAATACTGTAGT | |
| | GGTCATTAGTAAGCCCATACATTTCACAGTCCACT | |
| | AGACGATATGCTGGAAT | 1511 |
| | ATTCCAGCATATCGTCT | 1512 |
| Adenomatous polyposis | GACAAATTCCAACTCTAATTAGATGACCCATATTCTGTTTCTTA | 1513 |
| coli | CTAGGAATCAACCCTCAAAAGCGTATTGAGTGCCTTATGGAAT | |
| Lys586Term | TTGTCAGCACATTGCACTGAGAATAAAGCTGATA | |
| AAA-TAA | TATCAGCTTTATTCTCAGTGCAATGTGCTGACAAATTCCATAA | 1514 |
| | GGCACTCAATACGCTTTTGAGGGTTGATTCCTAGTAAGAAACA | |
| | GAATATGGGTCATCTAATTAGAGTTGGAATTTGTC | |
| | CAACCCTCAAAAGCGTA | 1515 |
| | TACGCTTTTGAGGGTTG | 1516 |
| Adenomatous polyposis | TAGATGACCCATATTCTGTTTCTTACTAGGAATCAACCCTCAA | 1517 |
| coli | AAGCGTATTGAGTGCCTTATGGAATTTGTCAGCACATTGCACT | |
| Leu592Term | GAGAATAAAGCTGATATATGTGCTGTAGATGGTGC | |
| TTA-TGA | GCACCATCTACAGCACATATATCAGCTTTATTCTCAGTGCAAT | 1518 |
| | GTGCTGACAAATTCCATAAGGCACTCAATACGCTTTTGAGGGT | |
| | TGATTCCTAGTAAGAAACAGAATATGGGTCATCTA | |
| | GAGTGCCTTATGGAATT | 1519 |
| | AATTCCATAAGGCACTC | 1520 |
| Adenomatous polyposis | ATGACCCATATTCTGTTTCTTACTAGGAATCAACCCTCAAAAG | 1521 |
| coli | CGTATTGAGTGCCTTATGGAATTTGTCAGCACATTGCACTGAG | |
| Trp593Term | AATAAAGCTGATATATGTGCTGTAGATGGTGCACT | |
| TGG-TAG | AGTGCACCATCTACAGCACATATATCAGCTTTATTCTCAGTGC | 1522 |
| | AATGTGCTGACAAATTCCATAAGGCACTCAATACGCTTTTGAG | |
| | GGTTGATTCCTAGTAAGAAACAGAATATGGGTCAT | |
| | TGCCTTATGGAATTTGT | 1523 |
| | ACAAATTCCATAAGGCA | 1524 |
| Adenomatous polyposis | TGACCCATATTCTGTTTCTTACTAGGAATCAACCCTCAAAAGC | 1525 |
| coli | GTATTGAGTGCCTTATGGAATTTGTCAGCACATTGCACTGAGA | |
| Trp593Term | ATAAAGCTGATATATGTGCTGTAGATGGTGCACTT | |
| TGG-TGA | AAGTGCACCATCTACAGCACATATATCAGCTTTATTCTCAGTG | 1526 |
| | CAATGTGCTGACAAATTCCATAAGGCACTCAATACGCTTTTGA | |
| | GGGTTGATTCCTAGTAAGAAACAGAATATGGGTCA | |
| | GCCTTATGGAATTTGTC | 1527 |
| | GACAAATTCCATAAGGC | 1528 |
| Adenomatous polyposis | TAAAGCTGATATATGTGCTGTAGATGGTGCACTTGCATTTTTG | 1529 |
| coli | GTTGGCACTCTTACTTACCGGAGCCAGACAAACACTTTAGCC | |
| Tyr622Term | ATTATTGAAAGTGGAGGTGGGATATTACGGAATGTG | |
| TAC-TAA | CACATTCCGTAATATCCCACCTCCACTTTCAATAATGGCTAAA | 1530 |
| | GTGTTTGTCTGGCTCCGGTAAGTAAGAGTGCCAACCAAAAAT | |
| | GCAAGTGCACCATCTACAGCACATATATCAGCTTTA | |
| | CTTACTTACCGGAGCCA | 1531 |
| | TGGCTCCGGTAAGTAAG | 1532 |
| Adenomatous polyposis | GATATATGTGCTGTAGATGGTGCACTTGCATTTTTGGTTGGCA | 1533 |
| coli | CTCTTACTTACCGGAGCCAGACAAACACTTTAGCCATTATTGA | |
| Gln625Term | AAGTGGAGGTGGGATATTACGGAATGTGTCCAGCT | |
| CAG-TAG | AGCTGGACACATTCCGTAATATCCCACCTCCACTTTCAATAAT | 1534 |
| | GGCTAAAGTGTTTGTCTGGCTCCGGTAAGTAAGAGTGCCAAC | |
| | CAAAAATGCAAGTGCACCATCTACAGCACATATATC | |
| | ACCGGAGCCAGACAAAC | 1535 |
| | GTTTGTCTGGCTCCGGT | 1536 |
| Adenomatous polyposis | TAGATGGTGCACTTGCATTTTTGGTTGGCACTCTTACTTACCG | 1537 |
| coli | GAGCCAGACAAACACTTTAGCCATTATTGAAAGTGGAGGTGG | |
| Leu629Term | GATATTACGGAATGTGTCCAGCTTGATAGCTACAAA | |
| TTA-TAA | TTTGTAGCTATCAAGCTGGACACATTCCGTAATATCCCACCTC | 1538 |
| | CACTTTCAATAATGGCTAAAGTGTTTGTCTGGCTCCGGTAAGT | |
| | AAGAGTGCCAACCAAAAATGCAAGTGCACCATCTA | |
| | AAACACTTTAGCCATTA | 1539 |
| | TAATGGCTAAAGTGTTT | 1540 |
| Adenomatous polyposis | GCCATTATTGAAAGTGGAGGTGGGATATTACGGAATGTGTCC | 1541 |
| coli | AGCTTGATAGCTACAAATGAGGACCACAGGTATATATAGAGTT | |
| Glu650Term | TTATATTACTTTTAAAGTACAGAATTCATACTCTCA | |
| GAG-TAG | TGAGAGTATGAATTCTGTACTTTAAAAGTAATATAAAACTCTAT | 1542 |
| | ATATACCTGTGGTCCTCATTTGTAGCTATCAAGCTGGACACAT | |
| | TCCGTAATATCCCACCTCCACTTTCAATAATGGC | |
| | CTACAAATGAGGACCAC | 1543 |
| | GTGGTCCTCATTTGTAG | 1544 |
| Adenomatous polyposis | TGCATGTGGAACTTTGTGGAATCTCTCAGCAAGAAATCCTAAA | 1545 |
| coli | GACCAGGAAGCATTATGGGACATGGGGGCAGTTAGCATGCTC | |
| Trp699Term | AAGAACCTCATTCATTCAAAGCACAAAATGATTGCT | |
| TGG-TGA | AGCAATCATTTTGTGCTTTGAATGAATGAGGTTCTTGAGCATG | 1546 |
| | CTAACTGCCCCCATGTCCCATAATGCTTCCTGGTCTTTAGGAT | |
| | TTCTTGCTGAGAGATTCCACAAAGTTCCACATGCA | |
| | GCATTATGGGACATGGG | 1547 |
| | CCCATGTCCCATAATGC | 1548 |
| Adenomatous polyposis | AAGACCAGGAAGCATTATGGGACATGGGGGCAGTTAGCATGC | 1549 |
| coli | TCAAGAACCTCATTCATTCAAAGCACAAAATGATTGCTATGGG | |
| Ser713Term | AAGTGCTGCAGCTTTAAGGAATCTCATGGCAAATAG | |
| TCA-TGA | CTATTTGCCATGAGATTCCTTAAAGCTGCAGCACTTCCCATAG | 1550 |
| | CAATCATTTTGTGCTTTGAATGAATGAGGTTCTTGAGCATGCT | |
| | AACTGCCCCCATGTCCCATAATGCTTCCTGGTCTT | |
| | CATTCATTCAAAGCACA | 1551 |
| | TGTGCTTTGAATGAATG | 1552 |
| Adenomatous polyposis | GGGGCAGTTAGCATGCTCAAGAACCTCATTCATTCAAAGCAC | 1553 |
| coli | AAAATGATTGCTATGGGAAGTGCTGCAGCTTTAAGGAATCTCA | |
| Ser722Gly | TGGCAAATAGGCCTGCGAAGTACAAGGATGCCAATA | |
| AGT-GGT | TATTGGCATCCTTGTACTTCGCAGGCCTATTTGCCATGAGATT | 1554 |
| | CCTTAAAGCTGCAGCACTTCCCATAGCAATCATTTTGTGCTTT | |
| | GAATGAATGAGGTTCTTGAGCATGCTAACTGCCCC | |
| | CTATGGGAAGTGCTGCA | 1555 |
| | TGCAGCACTTCCCATAG | 1556 |
| Adenomatous polyposis | TCTCCTGGCTCAGCTTGCCATCTCTTCATGTTAGGAAACAAAA | 1557 |
| coli | AGCCCTAGAAGCAGAATTAGATGCTCAGCACTTATCAGAAACT | |
| Leu764Term | TTTGACAATATAGACAATTTAAGTCCCAAGGCATC | |
| TTA-TAA | GATGCCTTGGGACTTAAATTGTCTATATTGTCAAAAGTTTCTG | 1558 |
| | ATAAGTGCTGAGCATCTAATTCTGCTTCTAGGGCTTTTTGTTT | |
| | CCTAACATGAAGAGATGGCAAGCTGAGCCAGGAGA | |
| | AGCAGAATTAGATGCTC | 1559 |
| | GAGCATCTAATTCTGCT | 1560 |
| Adenomatous polyposis | TTAGATGCTCAGCACTTATCAGAAACTTTTGACAATATAGACA | 1561 |
| coli | ATTTAAGTCCCAAGGCATCTCATCGTAGTAAGCAGAGACACA | |
| Ser784Thr | GCAAGTCTCTATGGTGATTATGTTTTTGACACCATC | |
| TCT-ACT | GATGGTGTCAAAAACATAATCACCATAGAGACTTGCTGTGTCT | 1562 |
| | CTGCTTACTACGATGAGATGCCTTGGGACTTAAATTGTCTATA | |
| | TTGTCAAAAGTTTCTGATAAGTGCTGAGCATCTAA | |
| | CCAAGGCATCTCATCGT | 1563 |
| | ACGATGAGATGCCTTGG | 1564 |
| Adenomatous polyposis | CTCATCGTAGTAAGCAGAGACACAGCAAGTCTCTATGGTGATT | 1565 |
| coli | ATGTTTTTGACACCAATCGACATGATGATAATAGGTCAGACAT | |
| Arg805Term | TTTAATACTGGCACATGACTGTCCTTTCACCATAT | |
| CGA-TGA | ATATGGTGAAAGGACAGTCATGTGCCAGTATTAAAATGTCTGA | 1566 |
| | CCTATTATCATCATGTCGATTGGTGTCAAAAACATAATCACCAT | |
| | AGAGACTTGCTGTGTCTCTGCTTACTACGATGAG | |
| | ACACCAATCGACATGAT | 1567 |
| | ATCATGTCGATTGGTGT | 1568 |
| Adenomatous polyposis | GGTCTAGGCAACTACCATCCAGCAACAGAAAATCCAGGAACT | 1569 |
| coli | TCTTCAAAGCGAGGTTTGCAGATCTCCACCACTGCAGCCCAG | |
| Gln879Term | ATTGCCAAAGTCATGGAAGAAGTGTCAGCCATTCATA | |
| CAG-TAG | TATGAATGGCTGACACTTCTTCCATGACTTTGGCAATCTGGGC | 1570 |
| | TGCAGTGGTGGAGATCTGCAAACCTCGCTTTGAAGAAGTTCC | |
| | TGGATTTTCTGTTGCTGGATGGTAGTTGCCTAGACC | |
| | GAGGTTTGCAGATCTCC | 1571 |
| | GGAGATCTGCAAACCTC | 1572 |
| Adenomatous polyposis | TACATTGTGTGACAGATGAGAGAAATGCACTTAGAAGAAGCTC | 1573 |
| coli | TGCTGCCCATACACATTCAAACACTTACAATTTCACTAAGTCG | |
| Ser932Term | GAAAATTCAAATAGGACATGTTCTATGCCTTATGC | |
| TCA-TAA | GCATAAGGCATAGAACATGTCCTATTTGAATTTTCCGACTTAG | 1574 |
| | TGAAATTGTAAGTGTTTGAATGTGTATGGGCAGCAGAGCTTCT | |
| | TCTAAGTGCATTTCTCTCATCTGTCACACAATGTA | |
| | TACACATTCAAACACTT | 1575 |
| | AAGTGTTTGAATGTGTA | 1576 |
| Adenomatous polyposis | TACATTGTGTGACAGATGAGAGAAATGCACTTAGAAGAAGCTC | 1577 |
| coli | TGCTGCCCATACACATTCAAACACTTACAATTTCACTAAGTCG | |
| Ser932Term | GAAAATTCAAATAGGACATGTTCTATGCCTTATGC | |
| TCA-TGA | GCATAAGGCATAGAACATGTCCTATTTGAATTTTCCGACTTAG | 1578 |
| | TGAAATTGTAAGTGTTTGAATGTGTATGGGCAGCAGAGCTTCT | |
| | TCTAAGTGCATTTCTCTCATCTGTCACACAATGTA | |
| | TACACATTCAAACACTT | 1579 |
| | AAGTGTTTGAATGTGTA | 1580 |
| Adenomatous polyposis | GACAGATGAGAGAAATGCACTTAGAAGAAGCTCTGCTGCCCA | 1581 |
| coli | TACACATTCAAACACTTACAATTTCACTAAGTCGGAAAATTCAA | |
| Tyr935Term | ATAGGACATGTTCTATGCCTTATGCCAAATTAGAA | |
| TAC-TAG | TTCTAATTTGGCATAAGGCATAGAACATGTCCTATTTGAATTTT | 1582 |
| | CCGACTTAGTGAAATTGTAAGTGTTTGAATGTGTATGGGCAGC | |
| | AGAGCTTCTTCTAAGTGCATTTCTCTCATCTGTC | |
| | AACACTTACAATTTCAC | 1583 |
| | GTGAAATTGTAAGTGTT | 1584 |
| Adenomatous polyposis | GACAGATGAGAGAAATGCACTTAGAAGAAGCTCTGCTGCCCA | 1585 |
| coli | TACACATTCAAACACTTACAATTTCACTAAGTCGGAAAATTCAA | |
| Tyr935Term | ATAGGACATGTTCTATGCCTTATGCCAAATTAGAA | |
| TAC-TAA | TTCTAATTTGGCATAAGGCATAGAACATGTCCTATTTGAATTTT | 1586 |
| | CCGACTTAGTGAAATTGTAAGTGTTTGAATGTGTATGGGCAGC | |
| | AGAGCTTCTTCTAAGTGCATTTCTCTCATCTGTC | |
| | AACACTTACAATTTCAC | 1587 |
| | GTGAAATTGTAAGTGTT | 1588 |
| Adenomatous polyposis | ACCCTCGATTGAATCCTATTCTGAAGATGATGAAAGTAAGTTT | 1589 |
| coli | TGCAGTTATGGTCAATACCCAGCCGACCTAGCCCATAAAATAC | |
| Tyr1000Term | ATAGTGCAAATCATATGGATGATAATGATGGAGAA | |
| TAC-TAA | TTCTCCATCATTATCATCCATATGATTTGCACTATGTATTTTAT | 1590 |
| | GGGCTAGGTCGGCTGGGTATTGACCATAACTGCAAAACTTAC | |
| | TTTCATCATCTTCAGAATAGGATTCAATCGAGGGT | |
| | GGTCAATACCCAGCCGA | 1591 |
| | TCGGCTGGGTATTGACC | 1592 |
| Adenomatous polyposis | TACCCAGCCGACCTAGCCCATAAAATACATAGTGCAAATCATA | 1593 |
| coli | TGGATGATAATGATGGAGAACTAGATACACCAATAAATTATAG | |
| Glu1020Term | TCTTAAATATTCAGATGAGCAGTTGAACTCTGGAA | |
| GAA-TAA | TTCCAGAGTTCAACTGCTCATCTGAATATTTAAGACTATAATTT | 1594 |
| | ATTGGTGTATCTAGTTCTCCATCATTATCATCCATATGATTTGC | |
| | ACTATGTATTTTATGGGCTAGGTCGGCTGGGTA | |
| | ATGATGGAGAACTAGAT | 1595 |
| | ATCTAGTTCTCCATCAT | 1596 |
| Adenomatous polyposis | ATGAAACCCTCGATTGAATCCTATTCTGAAGATGATGAAAGTA | 1597 |
| coli | AGTTTTGCAGTTATGGTCAATACCCAGCCGACCTAGCCCATAA | |
| Ser1032Term | AATACATAGTGCAAATCATATGGATGATAATGATG | |
| TCA-TAA | CATCATTATCATCCATATGATTTGCACTATGTATTTTATGGGCT | 1598 |
| | AGGTCGGCTGGGTATTGACCATAACTGCAAAACTTACTTTCAT | |
| | CATCTTCAGAATAGGATTCAATCGAGGGTTTCAT | |
| | GTTATGGTCAATACCCA | 1599 |
| | TGGGTATTGACCATAAC | 1600 |
| Adenomatous polyposis | TGAAGATGATGAAAGTAAGTTTTGCAGTTATGGTCAATACCCA | 1601 |
| coli | GCCGACCTAGCCCATAAAATACATAGTGCAAATCATATGGATG | |
| Gln1041Term | ATAATGATGGAGAACTAGATACACCAATAAATTAT | |
| CAA-TAA | ATAATTTATTGGTGTATCTAGTTCTCCATCATTATCATCCATAT | 1602 |
| | GATTTGCACTATGTATTTTATGGGCTAGGTCGGCTGGGTATTG | |
| | ACCATAACTGCAAAACTTACTTTCATCATCTTCA | |
| | GCCCATAAAATACATAG | 1603 |
| | CTATGTATTTTATGGGC | 1604 |
| Adenomatous polyposis | ATAAATTATAGTCTTAAATATTCAGATGAGCAGTTGAACTCTGG | 1605 |
| coli | AAGGCAAAGTCCTTCACAGAATGAAAGATGGGCAAGACCCAA | |
| Gln1045Term | ACACATAATAGAAGATGAAATAAAACAAAGTGAGC | |
| CAG-TAG | GCTCACTTTGTTTTATTTCATCTTCTATTATGTGTTTGGGTCTT | 1606 |
| | GCCCATCTTTCATTCTGTGAAGGACTTTGCCTTCCAGAGTTCA | |
| | ACTGCTCATCTGAATATTTAAGACTATAATTTAT | |
| | GTCCTTCACAGAATGAA | 1607 |
| | TTCATTCTGTGAAGGAC | 1608 |
| Adenomatous polyposis | GAAAGATGGGCAAGACCCAAACACATAATAGAGATGAAATA | 1609 |
| coli | AAACAAAGTGAGCAAAGACAATCAAGGAATCAAAGTACAACTT | |
| Gln1067Term | ATCCTGTTTATACTGAGAGCACTGATGATAAACACC | |
| CAA-TAA | GGTGTTTATCATCAGTGCTCTCAGTATAAACAGGATAAGTTGT | 1610 |
| | ACTTTGATTCCTTGATTGTCTTTGCTCACTTTGTTTTATTTCATC | |
| | TTCTATTATGTGTTTGGGTCTTGCCCATCTTTC | |
| | AGCAAAGACAATCAAGG | 1611 |
| | CCTTGATTGTCTTTGCT | 1612 |
| Adenomatous polyposis | AATAGAAGATGAAATAAAACAAAGTGAGCAAAGACAATCAAGG | 1613 |
| coli | AATCAAAGTACAACTTATCCTGTTTATACTGAGAGCACTGATG | |
| Tyr1075Term | ATAAACACCTCAAGTTCCAACCACATTTTGGACAG | |
| TAT-TAG | CTGTCCAAAATGTGGTTGGAACTTGAGGTGTTTATCATCAGTG | 1614 |
| | CTCTCAGTATAAACAGGATAAGTTGTACTTTGATTCCTTGATT | |
| | GTCTTTGCTCACTTTGTTTTATTTCATCTTCTATT | |
| | ACAACTTATCCTGTTTA | 1615 |
| | TAAACAGGATAAGTTGT | 1616 |
| Adenomatous polyposis | TGATGATAAACACCTCAAGTTCCAACCACATTTTGGACAGCAG | 1617 |
| coli | GAATGTGTTTCTCCATACAGGTCACGGGGAGCCAATGGTTCA | |
| Tyr1102Term | GAAACAAATCGAGTGGGTTCTAATCATGGAATTAAT | |
| TAC-TAG | ATTAATTCCATGATTAGAACCCACTCGATTTGTTTCTGAACCAT | 1618 |
| | TGGCTCCCCGTGACCTGTATGGAGAAACACATTCCTGCTGTC | |
| | CAAAATGTGGTTGGAACTTGAGGTGTTTATCATCA | |
| | TCTCCATACAGGTCACG | 1619 |
| | CGTGACCTGTATGGAGA | 1620 |
| Adenomatous polyposis | AACCACATTTTGGACAGCAGGAATGTGTTTCTCCATACAGGTC | 1621 |
| coli | ACGGGGAGCCAATGGTTCAGAAACAAATCGAGTGGGTTCTAA | |
| Ser1110Term | TCATGGAATTAATCAAAATGTAAGCCAGTCTTTGTG | |
| TCA-TGA | CACAAAGACTGGCTTACATTTTGATTAATTCCATGATTAGAAC | 1622 |
| | CCACTCGATTTGTTTCTGAACCATTGGCTCCCCGTGACCTGTA | |
| | TGGAGAAACACATTCCTGCTGTCCAAAATGTGGTT | |
| | CAATGGTTCAGAAACAA | 1623 |
| | TTGTTTCTGAACCATTG | 1624 |
| Adenomatous polyposis | GGACAGCAGGAATGTGTTTCTCCATACAGGTCACGGGGAGCC | 1625 |
| coli | AATGGTTCAGAAACAAATCGAGTGGGTTCTAATCATGGAATTA | |
| Arg1114Term | ATCAAAATGTAAGCCAGTCTTTGTGTCAAGAAGATG | |
| CGA-TGA | CATCTTCTTGACACAAAGACTGGCTTACATTTTGATTAATTCCA | 1626 |
| | TGATTAGAACCCACTCGATTTGTTTCTGAACCATTGGCTCCCC | |
| | GTGACCTGTATGGAGAAACACATTCCTGCTGTCC | |
| | AAACAAATCGAGTGGGT | 1627 |
| | ACCCACTCGATTTGTTT | 1628 |
| Adenomatous polyposis | GGGTTCTAATCATGGAATTAATCAAAATGTAAGCCAGTCTTTG | 1629 |
| coli | TGTCAAGAAGATGACTATGAAGATGATAAGCCTACCAATTATA | |
| Tyr1135Term | GTGAACGTTACTCTGAAGAAGAACAGCATGAAGAA | |
| TAT-TAG | TTCTTCATGCTGTTCTTCTTCAGAGTAACGTTCACTATAATTGG | 1630 |
| | TAGGCTTATCATCTTCATAGTCATCTTCTTGACACAAAGACTG | |
| | GCTTACATTTTGATTAATTCCATGATTAGAACCC | |
| | GATGACTATGAAGATGA | 1631 |
| | TCATCTTCATAGTCATC | 1632 |
| Adenomatous polyposis | GAAGATGACTATGAAGATGATAAGCCTACCAATTATAGTGAAC | 1633 |
| coli | GTTACTCTGAAGAAGAACAGCATGAAGAAGAAGAGAGACCAA | |
| Gln1152Term | CAAATTATAGCATAAAATATAATGAAGAGAAACGTC | |
| CAG-TAG | GACGTTTCTCTTCATTATATTTTATGCTATAATTTGTTGGTCTC | 1634 |
| | TCTTCTTCTTCATGCTGTTCTTCTTCAGAGTAACGTTCACTATA | |
| | ATTGGTAGGCTTATCATCTTCATAGTCATCTTC | |
| | AAGAAGAACAGCATGAA | 1635 |
| | TTCATGCTGTTCTTCTT | 1636 |
| Adenomatous polyposis | GAAGAAGAGAGACCAACAAATTATAGCATAAAATATAATGAAG | 1637 |
| coli | AGAAACGTCATGTGGATCAGCCTATTGATTATAGTTTAAAATAT | |
| Gln1175Term | GCCACAGATATTCCTTCATCACAGAAACAGTCAT | |
| CAG-TAG | ATGACTGTTTCTGTGATGAAGGAATATCTGTGGCATATTTTAA | 1638 |
| | ACTATAATCAATAGGCTGATCCACATGACGTTTCTCTTCATTAT | |
| | ATTTTATGCTATAATTTGTTGGTCTCTCTTCTTC | |
| | ATGTGGATCAGCCTATT | 1639 |
| | AATAGGCTGATCCACAT | 1640 |
| Adenomatous polyposis | AAGAGAGACCAACAAATTATAGCATAAAATATAATGAAGAGAA | 1641 |
| coli | ACGTCATGTGGATCAGCCTATTGATTATAGTTTAAAATATGCC | |
| Pro1176Leu | ACAGATATTCCTTCATCACAGAAACAGTCATTTTC | |
| CCT-CTT | GAAAATGACTGTTTCTGTGATGAAGGAATATCTGTGGCATATT | 1642 |
| | TTAAACTATAATCAATAGGCTGATCCACATGACGTTTCTCTTCA | |
| | TTATATTTTATGCTATAATTTGTTGGTCTCTCTT | |
| | GGATCAGCCTATTGATT | 1643 |
| | AATCAATAGGCTGATCC | 1644 |
| Adenomatous polyposis | ATAAAATATAATGAAGAGAAACGTCATGTGGATCAGCCTATTG | 1645 |
| coli | ATTATAGTTTAAAATATGCCACAGATATTCCTTCATCACAGAAA | |
| Ala1184Pro | CAGTCATTTTCATTCTCAAAGAGTTCATCTGGAC | |
| GCC-CCC | GTCCAGATGAACTCTTTGAGAATGAAAATGACTGTTTCTGTGA | 1646 |
| | TGAAGGAATATCTGTGGCATATTTTAAACTATAATCAATAGGCT | |
| | GATCCACATGACGTTTCTCTTCATTATATTTTAT | |
| | TAAAATATGCCACAGAT | 1647 |
| | ATCTGTGGCATATTTTA | 1648 |
| Adenomatous polyposis | ATCAGCCTATTGATTATAGTTTAAAATATGCCACAGATATTCCT | 1649 |
| coli | TCATCACAGAAACAGTCATTTTCATTCTCAAAGAGTTCATCTG | |
| Ser1194Term | GACAAAGCAGTAAAACCGAACATATGTCTTCAAG | |
| TCA-TGA | CTTGAAGACATATGTTCGGTTTTACTGCTTTGTCCAGATGAAC | 1650 |
| | TCTTTGAGAATGAAAATGACTGTTTCTGTGATGAAGGAATATC | |
| | TGTGGCATATTTTAAACTATAATCAATAGGCTGAT | |
| | GAAACAGTCATTTTCAT | 1651 |
| | ATGAAAATGACTGTTTC | 1652 |
| Adenomatous polyposis | ATTATAGTTTAAAATATGCCACAGATATTCCTTCATCACAGAAA | 1653 |
| coli | CAGTCATTTTCATTCTCAAAGAGTTCATCTGGACAAAGCAGTA | |
| Ser1198Term | AAACCGAACATATGTCTTCAAGCAGTGAGAATAC | |
| TCA-TGA | GTATTCTCACTGCTTGAAGACATATGTTCGGTTTTACTGCTTT | 1654 |
| | GTCCAGATGAACTCTTTGAGAATGAAAATGACTGTTTCTGTGA | |
| | TGAAGGAATATCTGTGGCATATTTTAAACTATAAT | |
| | TTCATTCTCAAAGAGTT | 1655 |
| | AACTCTTTGAGAATGAA | 1656 |
| Adenomatous polyposis | ACCGAACATATGTCTTCAAGCAGTGAGAATACGTCCACACCTT | 1657 |
| coli | CATCTAATGCCAAGAGGCAGAATCAGCTCCATCCAGTTCTGC | |
| Gln1228Term | ACAGAGTAGAAGTGGTCAGCCTCAAAGGCTGCCACT | |
| CAG-TAG | AGTGGCAGCCTTTGAGGCTGACCACTTCTACTCTGTGCAGAA | 1658 |
| | CTGGATGGAGCTGATTCTGCCTCTTGGCATTAGATGAAGGTG | |
| | TGGACGTATTCTCACTGCTTGAAGACATATGTTCGGT | |
| | CCAAGAGGCAGAATCAG | 1659 |
| | CTGATTCTGCCTCTTGG | 1660 |
| Adenomatous polyposis | CATATGTCTTCAAGCAGTGAGAATACGTCCACACCTTCATCTA | 1661 |
| coli | ATGCCAAGAGGCAGAATCAGCTCCATCCAGTTCTGCACAGAG | |
| Gln1230Term | TAGAAGTGGTCAGCCTCAAAGGCTGCCACTTGCAAG | |
| CAG-TAG | CTTGCAAGTGGCAGCCTTTGAGGCTGACCACTTCTACTCTGT | 1662 |
| | GCAGAACTGGATGGAGCTGATTCTGCCTCTTGGCATTAGATG | |
| | AAGGTGTGGACGTATTCTCACTGCTTGAAGACATATG | |
| | GGCAGAATCAGCTCCAT | 1663 |
| | ATGGAGCTGATTCTGCC | 1664 |
| Adenomatous polyposis | TCAGCTCCATCCAAGTTCTGCACAGAGTAGAAGTGGTCAGCC | 1665 |
| coli | TCAAAAGGCTGCCACTTGCAAAGTTTCTTCTATTAACCAAGAA | |
| Cys1249Term | ACAATACAGACTTATTGTGTAGAAGATACTCCAATA | |
| TGC-TGA | TATTGGAGTATCTTCTACACAATAAGTCTGTATTGTTTCTTGGT | 1666 |
| | TAATAGAAGAAACTTTGCAAGTGGCAGCCTTTTGAGGCTGAC | |
| | CACTTCTACTCTGTGCAGAACTTGGATGGAGCTGA | |
| | GCCACTTGCAAAGTTTC | 1667 |
| | GAAACTTTGCAAGTGGC | 1668 |
| Adenomatous polyposis | AGTTTCTTCTATTAACCAAGAAACAATACAGACTTATTGTGTAG | 1669 |
| coli | AAGATACTCCAATATGTTTTTCAAGATGTAGTTCATTATCATCT | |
| Cys1270Term | TTGTCATCAGCTGAAGATGAAATAGGATGTAAT | |
| TGT-TGA | ATTACATCCTATTTCATCTTCAGCTGATGACAAAGATGATAATG | 1670 |
| | AACTACATCTTGAAAAACATATTGGAGTATCTTCTACACAATAA | |
| | GTCTGTATTGTTTCTTGGTTAATAGAAGAAACT | |
| | CCAATATGTTTTTCAAG | 1671 |
| | CTTGAAAAACATATTGG | 1672 |
| Adenomatous polyposis | AAGAAACAATACAGACTTATTGTGTAGAAGATACTCCAATATG | 1673 |
| coli | TTTTTCAAGATGTAGTTCATTATCATCTTTGTCATCAGCTGAAG | |
| Ser1276Term | ATGAAATAGGATGTAATCAGACGACACAGGAAGC | |
| TCA-TGA | GCTTCCTGTGTCGTCTGATTACATCCTATTTCATCTTCAGCTG | 1674 |
| | ATGACAAAGATGATAATGAACTACATCTTGAAAAACATATTGG | |
| | AGTATCTTCTACACAATAAGTCTGTATTGTTTCTT | |
| | ATGTAGTTCATTATCAT | 1675 |
| | ATGATAATGAACTACAT | 1676 |
| Adenomatous polyposis | GATACTCCAATATGTTTTTCAAGATGTAGTTCATTATCATCTTT | 1677 |
| coli | GTCATCAGCTGAAGATGAAATAGGATGTAATCAGACGACACA | |
| Glu1286Term | GGAAGCAGATTCTGCTAATACCCTGCAAATAGCAG | |
| GAA-TAA | CTGCTATTTGCAGGGTATTAGCAGAATCTGCTTCCTGTGTCGT | 1678 |
| | CTGATTACATCCTATTTCATCTTCAGCTGATGACAAAGATGATA | |
| | ATGAACTACATCTTGAAAAACATATTGGAGTATC | |
| | CTGAAGATGAAATAGGA | 1679 |
| | TCCTATTTCATCTTCAG | 1680 |
| Adenomatous polyposis | TGTAGTTCATTATCATCTTTGTCATCAGCTGAAGATGAAATAG | 1681 |
| coli | GATGTAATCAGACGACACAGGAAGCAGATTCTGCAAATACCC | |
| Gln1294Term | TGCAAATAGCAGAAATAAAAGAAAAGATTGGAACTA | |
| CAG-TAG | TAGTTCCAATCTTTTCTTTTATTTCTGCTATTTGCAGGGTATTA | 1682 |
| | GCAGAATCTGCTTCCTGTGTCGTCTGATTACATCCTATTTCAT | |
| | CTTCAGCTGATGACAAAGATGATAATGAACTACA | |
| | AGACGACACAGGAAGCA | 1683 |
| | TGCTTCCTGTGTCGTCT | 1684 |
| Predisposition to, | TAGGATGTAATCAGACGACACAGGAAGCAGATTCTGCTAATA | 1685 |
| association with, | CCCTGCAAATAGCAGAAATAAAAGAAAAGATTGGAACTAGGTC | |
| colorectal cancer | AGCTGAAGATCCTGTGAGCGAAGTTCCAGCAGTGTC | |
| Ile1307Lys | GACACTGCTGGAACTTCGCTCACAGGATCTTCAGCTGACCTA | 1686 |
| ATA-AAA | GTTCCAATCTTTTCTTTTATTTCTGCTATTTGCAGGGTATTAGC | |
| | AGAATCTGCTTCCTGTGTCGTCTGATTACATCCTA | |
| | AGCAGAAATAAAAGAAA | 1687 |
| | TTTCTTTTATTTCTGCT | 1688 |
| Adenomatous polyposis | CCAAGAAACAATACAGACTTATTGTGTAGAAGATACTCCAATA | 1689 |
| coli | TGTTTTTCAAGATGTAGTTCATTATCATCTTTGTCATCAGCTGA | |
| Glu1309Term | AGATGAAATAGGATGTAATCAGACGACACAGGAA | |
| GAA-TAA | TTCCTGTGTCGTCTGATTACATCCTATTTCATCTTCAGCTGATG | 1690 |
| | ACAAAGATGATAATGAACTACATCTTGAAAAACATATTGGAGT | |
| | ATCTTCTACACAATAAGTCTGTATTGTTTCTTGG | |
| | AGATGTAGTTCATTATC | 1691 |
| | GATAATGAACTACATCT | 1692 |
| Predisposition to | GATTCTGCTAATACCCTGCAAATAGCAGAAATAAAAGAAAAGA | 1693 |
| Colorectal Cancer | TTGGAACTAGGTCAGCTGAAGATCCTGTGAGCGAAGTTCCAG | |
| Glu1317Gln | CAGTGTCACAGCACCCTAGAACCAAATCCAGCAGAC | |
| GAA-CAA | GTCTGCTGGATTTGGTTCTAGGGTGCTGTGACACTGCTGGAA | 1694 |
| | CTTCGCTCACAGGATCTTCAGCTGACCTAGTTCCAATCTTTTC | |
| | TTTTATTTCTGCTATTTGCAGGGTATTAGCAGAATC | |
| | GGTCAGCTGAAGATCCT | 1695 |
| | AGGATCTTCAGCTGACC | 1696 |
| Adenomatous polyposis | AAAGAAAAGATTGGAACTAGGTCAGCTGAAGATCCTGTGAGC | 1697 |
| coli | GAAGTTCCAGCAGTGTCACAGCACCCTAGAACCAAATCCAGC | |
| Gln1328Term | AGACTGCAGGGTTCTAGTTTATCTTCAGAATCAGCCA | |
| CAG-TAG | TGGCTGATTCTGAAGATAAACTAGAACCCTGCAGTCTGCTGG | 1698 |
| | ATTTGGTTCTAGGGTGCTGTGACACTGCTGGAACTTCGCTCA | |
| | CAGGATCTTCAGCTGACCTAGTTCCAATCTTTTCTTT | |
| | CAGTGTCACAGCACCCT | 1699 |
| | AGGGTGCTGTGACACTG | 1700 |
| Adenomatous polyposis | GATCCTGTGAGCGAAGTTCCAGCAGTGTCACAGCACCCTAGA | 1701 |
| coli | ACCAAATCCAGCAGACTGCAGGGTTCTAGTTTATCTTCAGAAT | |
| Gln1338Term | CAGCCAGGCACAAAGCTGTTGAATTTTCTTCAGGAG | |
| CAG-TAG | CTCCTGAAGAAAATTCAACAGCTTTGTGCCTGGCTGATTCTGA | 1702 |
| | AGATAAACTAGAACCCTGCAGTCTGCTGGATTTGGTTCTAGG | |
| | GTGCTGTGACACTGCTGGAACTTCGCTCACAGGATC | |
| | GCAGACTGCAGGGTTCT | 1703 |
| | AGAACCCTGCAGTCTGC | 1704 |
| Adenomatous polyposis | AAGTTCCAGCAGTGTCACAGCACCCTAGAACCAAATCCAGCA | 1705 |
| coli | GACTGCAGGGTTCTAGTTTATCTTCAGAATCAGCCAGGCACA | |
| Leu1342Term | AAGCTGTTGAATTTTCTTCAGGAGCGAAATCTCCCTC | |
| TTA-TAA | GAGGGAGATTTCGCTCCTGAAGAAAATTCAACAGCTTTGTGC | 1706 |
| | CTGGCTGATTCTGAAGATAAACTAGAACCCTGCAGTCTGCTG | |
| | GATTTGGTTCTAGGGTGCTGTGACACTGCTGGAACTT | |
| | TTCTAGTTTATCTTCAG | 1707 |
| | CTGAAGATAAACTAGAA | 1708 |
| Adenomatous polyposis | CAGCACCCTAGAACCAAATCCAGCAGACTGCAGGGTTCTAGT | 1709 |
| coli | TTATCTTCAGAATCAGCCAGGCACAAAGCTGTTGAATTTTCTT | |
| Arg1348Trp | CAGGAGCGAAATCTCCCTCCCGAAAGTGGTGCTCAG | |
| AGG-TGG | CTGAGCACCACTTTCGGGAGGGAGATTTCGCTCCTGAAGAAA | 1710 |
| | ATTCAACAGCTTTGTGCCTGGCTGATTCTGAAGATAAACTAGA | |
| | ACCCTGCAGTCTGCTGGATTTGGTTCTAGGGTGCTG | |
| | AATCAGCCAGGCACAAA | 1711 |
| | TTTGTGCCTGGCTGATT | 1712 |
| Adenomatous polyposis | CTGCAGGGTTCTAGTTTATCTTCAGAATCAGCCAGGCACAAA | 1713 |
| coli | GCTGTTGAATTTTCTTCAGGAGCGAAATCTCCCTCCCGAAAGT | |
| Gly1357Term | GGTGCTCAGACACCCCAAAGTCCACCTGAACACTAT | |
| GGA-TGA | ATAGTGTTCAGGTGGACTTTGGGGTGTCTGAGCACCACTTTC | 1714 |
| | GGGAGGGAGATTTCGCTCCTGAAGAAAATTCAACAGCTTTGT | |
| | GCCTGGCTGATTCTGAAGATAAACTAGAACCCTGCAG | |
| | TTTCTTCAGGAGCGAAA | 1715 |
| | TTTCGCTCCTGAAGAAA | 1716 |
| Adenomatous polyposis | CCAGGCACAAAGCTGTTGAATTTTCTTCAGGAGCGAAATCTCC | 1717 |
| coli | CTCCCGAAAGTGGTGCTCAGACACCCCAAAGTCCACCTGAAC | |
| Gln1367Term | ACTATGTTCAGGAGACCCCACTCATGTTTAGCAGAT | |
| CAG-TAG | ATCTGCTAAACATGAGTGGGGTCTCCTGAACATAGTGTTCAG | 1718 |
| | GTGGACTTTGGGGTGTCTGAGCACCACTTTCGGGAGGGAGAT | |
| | TTCGCTCCTGAAGAAAATTCAACAGCTTTGTGCCTGG | |
| | GTGGTGCTCAGACACCC | 1719 |
| | GGGTGTCTGAGCACCAC | 1720 |
| Adenomatous polyposis | AAAGCTGTTGAATTTTCTTCAGGAGCGAAATCTCCCTCCAAAA | 1721 |
| coli | GTGGTGCTCAGACACCCAAAAGTCCACCTGAACACTATGTTC | |
| Lys1370Term | AGGAGACCCCACTCATGTTTAGCAGATGTACTTCTG | |
| AAA-TAA | CAGAAGTACATCTGCTAAACATGAGTGGGGTCTCCTGAACAT | 1722 |
| | AGTGTTCAGGTGGACTTTBGGGTGTCTGAGCACCACTTTTGG | |
| | AGGGAGATTTCGCTCCTGAAGAAAATTCAACAGCTTT | |
| | AGACACCCBAAAGTCCA | 1723 |
| | TGGACTTTTGGGTGTCT | 1724 |
| Adenomatous polyposis | CACCTGAACACTATGTTCAGGAGACCCCACTCATGTTTAGCA | 1725 |
| coli | GATGTACTTCTGTCAGTTCACTTGATAGTTTTGAGAGTCGTTC | |
| Ser1392Term | GATTGCCAGCTCCGTTCAGAGTGAACCATGCAGTGG | |
| TCA-TAA | CCACTGCATGGTTCACTCTGAACGGAGCTGGCAATCGAACGA | 1726 |
| | CTCTCAAAACTATCAAGTGAACTGACAGAAGTACATCTGCTAA | |
| | ACATGAGTGGGGTCTCCTGAACATAGTGTTCAGGTG | |
| | TGTCAGTTCACTTGATA | 1727 |
| | TATCAAGTGAACTGACA | 1728 |
| Adenomatous polyposis | CACCTGAACACTATGTTCAGGAGACCCCACTCATGTTTAGCA | 1729 |
| coli | GATGTACTTCTGTCAGTTCACTTGATAGTTTTGAGAGTCGTTC | |
| Ser1392Term | GATTGCCAGCTCCGTTCAGAGTGAACCATGCAGTGG | |
| TCA-TGA | CCACTGCATGGTTCACTCTGAACGGAGCTGGCAATCGAACGA | 1730 |
| | CTCTCAAAACTATCAAGTGAACTGACAGAAGTACATCTGCTAA | |
| | ACATGAGTGGGGTCTCCTGAACATAGTGTTCAGGTG | |
| | TGTCAGTTCACTTGATA | 1731 |
| | TATCAAGTGAACTGACA | 1732 |
| Adenomatous polyposis | GTTCAGGAGACCCCACTCATGTTTAGCAGATGTACTTCTGTCA | 1733 |
| coli | GTTCACTTGATAGTTTTGAGAGTCGTTCGATTGCCAGCTCCGT | |
| Glu1397Term | TCAGAGTGAACCATGCAGTGGAATGGTAGGTGGCA | |
| GAG-TAG | TGCCACCTACCATTCCACTGCATGGTTCACTCTGAACGGAGC | 1734 |
| | TGGCAATCGAACGACTCTCAAAACTATCAAGTGAACTGACAGA | |
| | AGTACATCTGCTAAACATGAGTGGGGTCTCCTGAAC | |
| | ATAGTTTTGAGAGTCGT | 1735 |
| | ACGACTCTCAAAACTAT | 1736 |
| Adenomatous polyposis | CAAACCATGCCACCAAGCAGAAGTAAAACACCTCCACCACCT | 1737 |
| coli | CCTCAAACAGCTCAAACCAAGCGAGAAGTACCTAAAAATAAA | |
| Lys1449Term | GCACCTACTGCTGAAAAGAGAGAGAGTGGACCTAAGC | |
| AAG-TAG | GCTTAGGTCCACTCTCTCTCTTTTCAGCAGTAGGTGCTTTATT | 1738 |
| | TTTAGGTACTTCTCGCTTGGTTTGAGCTGTTTGAGGAGGTGGT | |
| | GGAGGTGTTTACTTCTGCTTGGTGGCATGGTTTG | |
| | CTCAAACCAAGCGAGAA | 1739 |
| | TTCTCGCTTGGTTTGAG | 1740 |
| Adenomatous polyposis | ACCATGCCACCAAGCAGAAGTAAAACACCTCCACCACCTCCT | 1741 |
| coli | CAAACAGCTCAAACCAAGCGAGAAGTACCTAAAAATAAAGCA | |
| Arg1450Term | CCTACTGCTGAAAAGAGAGAGAGTGGACCTAAGCAAG | |
| CGA-TGA | CTTGCTTAGGTCCACTCTCTCTCTTTTCAGCAGTAGGTGCTTT | 1742 |
| | ATTTTTAGGTACTTCTCGCTTGGTTTGAGCTGTTTGAGGAGGT | |
| | GGTGGAGGTGTTTTACTTCTGCTTGGTGGCATGGT | |
| | AAACCAAGCGAGAAGTA | 1743 |
| | TACTTCTCGCTTGGTTT | 1744 |
| Adenomatous polyposis | CAGATGCTGATACTTTATTACATTTTGCCACGGAAAGTACTCC | 1745 |
| coli | AGATGGATTTTCTTGTTCATCCAGCCTGAGTGCTCTGAGCCTC | |
| Ser1503Term | GATGAGCCATTTATACAGAAAGATGTGGAATTAAG | |
| TCA-TAA | CTTAATTCCACATCTTTCTGTATAAATGGCTCATCGAGGCTCA | 1746 |
| | GAGCACTCAGGCTGGATGAACAAGAAAATCCATCTGGAGTAC | |
| | TTTCCGTGGCAAAATGTAATAAAGTATCAGCATCTG | |
| | TTCTTGTTCATCCAGCC | 1747 |
| | GGCTGGATGAACAAGAA | 1748 |
| Adenomatous polyposis | CTGAGCCTCGATGAGCCATTTATACAGAAAGATGTGGAATTAA | 1749 |
| coli | GAATAATGCCTCCAGTTCAGGAAAATGACAATGGGAATGAAA | |
| Gln1529Term | CAGAATCAGAGCAGCCTAAAGAATCAAATGAAAACC | |
| CAG-TAG | GGTTTTCATTTGATTCTTTAGGCTGCTCTGATTCTGTTTCATTC | 1750 |
| | CCATTGTCATTTTCCTGAACTGGAGGCATTATTCTTAATTCCAC | |
| | ATCTTTCTGTATAAATGGCTCATCGAGGCTCAG | |
| | CTCCAGTTCAGGAAAAT | 1751 |
| | ATTTTCCTGAACTGGAG | 1752 |
| Adenomatous polyposis | ATGTGGAATTAAGAATAATGCCTCCAGTTCAGGAAAATGACAA | 1753 |
| coli | TGGGAATGAAACAGAATCAGAGCAGCCTAAAGAATCAAATGA | |
| Ser1539Term | AAACCAAGAGAAAGAGGCAGAAAAAACTATTGATTC | |
| TCA-TAA | GAATCAATAGTTTTTTCTGCCTCTTTCTCTTGGTTTTCATTTGA | 1754 |
| | TTCTTTAGGCTGCTCTGATTCTGTTTCATTCCCATTGTCATTTT | |
| | CCTGAACTGGAGGCATTATTCTTAATTCCACAT | |
| | AACAGAATCAGAGCAGC | 1755 |
| | GCTGCTCTGATTCTGTT | 1756 |
| Adenomatous polyposis | AAAACCAAGAGAAAGAGGCAGAAAAAACTATTGATTCTGAAAA | 1757 |
| coli | GGACCTATTAGATGATTCAGATGATGATGATATTGAAATACTA | |
| Ser1567Term | GAAGAATGTATTATTTCTGCCATGCCAACAAAGTC | |
| TCA-TGA | GACTTTGTTGGCATGGCAGAAATAATACATTCTTCTAGTATTTC | 1758 |
| | AATATCATCATCATCTGAATCATCTAATAGGTCCTTTTCAGAAT | |
| | CAATAGTTTTTTCTGCCTCTTTCTCTTGGTTTT | |
| | AGATGATTCAGATGATG | 1759 |
| | CATCATCTGAATCATCT | 1760 |
| Adenomatous polyposis | AGAGAGTTTTCTCAGACAACAAAGATTCAAAGAAACAGAATTT | 1761 |
| coli | GAAAAATAATTCCAAGGACTTCAATGATAAGCTCCCAAATAAT | |
| Asp1822Val | GAAGATAGAGTCAGAGGAAGTTTTGCTTTTGATTC | |
| GAC-GTC | GAATCAAAAGCAAAACTTCCTCTGACTCTATCTTCATTATTTGG | 1762 |
| | GAGCTTATCATTGAAGTCCTTGGAATTATTTTTCAAATTCTGTT | |
| | TCTTTGAATCTTTGTTGTCTGAGAAAACTCTCT | |
| | TTCCAAGGACTTCAATG | 1763 |
| | CATTGAAGTCCTTGGAA | 1764 |
| Adenomatous polyposis | AAAACTGACAGCACAGAATCCAGTGGAACCCAAAGTCCTAAG | 1765 |
| coli | CGCCATTCTGGGTCTTACCTTGTGACATCTGTTTAAAAGAGAG | |
| Leu2839Phe | GAAGAATGAAACTAAGAAAATTCTATGTTAATTACA | |
| CTT-TTT | TGTAATTAACATAGAATTTTCTTAGTTTCATTCTTCCTCTCTTTT | 1766 |
| | AAACAGATGTCACAAGGTAAGACCCAGAATGGCGCTTAGGAC | |
| | TTTGGGTTCCACTGGATTCTGTGCTGTCAGTTTT | |
| | GGTCTTACCTTGTGACA | 1767 |
| | TGTCACAAGGTAAGACC | 1768 |

### EXAMPLE 13

### Parahemophilia—Factor V Deficiency

Deficiency in clotting Factor V is associated with a lifelong predisposition to thrombosis. The disease typically manifests itself with usually mild bleeding, although bleeding times and clotting times are consistently prolonged. Individuals that are heterozygous for a mutation in Factor V have lowered levels of factor V but probably never have abnormal bleeding. A large number of alleles with a range of presenting symptoms have been identified. The attached table discloses the correcting oligonucleotide base sequences for the Factor V oligonucleotides of the invention.

TABLE 15
Factor V Mutations and Genome-Correcting Oligos

| Clinical Phenotype & | | SEQ ID |
|---|---|---|
| Mutation | Correcting Oligos | NO: |
| Factor V deficiency | TTGACTGAATGCTTATTTTGGCCTGTGTCTCTCCCTCTTTCTCA | 4340 |
| Ala221Val | GATATAACAGTTTGTGCCCATGACCACATCAGCTGGCATCTGC | |
| GCC-GTC | TGGGAATGAGCTCGGGGCCAGAATTATTCTCCAT | |
| | ATGGAGAATAATTCTGGCCCCGAGCTCATTCCCAGCAGATGC | 1769 |
| | CAGCTGATGTGGTCATGGGCACAAACTGTTATATCTGAGAAA | |
| | GAGGGAGAGACACAGGCCAAAATAAGCATTCAGTCAA | |
| | AGTTTGTGCCCATGACC | 1770 |
| | GGTCATGGGCACAAACT | 1771 |
| Thrombosis | TGTCCTAACTCAGCTGGGATGCAGGCTTACATTGACATTAAAA | 1772 |
| Arg306Gly | ACTGCCCAAAGAAAACCAGGAATCTTAAGAAAATAACTCGTGA | |
| AGG-GGG | GCAGAGGCGGCACATGAAGAGGTGGGAATACTTCA | |
| | TGAAGTATTCCCACCTCTTCATGTGCCGCCTCTGCTCACGAGT | 1773 |
| | TATTTTCTTAAGATTCCTGGTTTTCTTTGGGCAGTTTTTAATGT | |
| | CAATGTAAGCCTGCATCCCAGCTGAGTTAGGACA | |
| | AGAAAACCAGGAATCTT | 1774 |
| | AAGATTCCTGGTTTTCT | 1775 |
| Thrombosis | GTCCTAACTCAGCTGGGATGCAGGCTTACATTGACATTAAAAA | 1776 |
| Arg306Thr | CTGCCCAAAGAAAACCAGGAATCTTAAGAAAATAACTCGTGAG | |
| AGG-ACG | CAGAGGCGGCACATGAAGAGGTGGGAATACTTCAT | |
| | ATGAAGTATTCCCACCTCTTCATGTGCCGCCTCTGCTCACGAG | 1777 |
| | TTATTTTCTTAAGATTCCTGGTTTTCTTTGGGCAGTTTTTAATG | |
| | TCAATGTAAGCCTGCATCCCAGCTGAGTTAGGAC | |
| | GAAAACCAGGAATCTTA | 1778 |
| | TAAGATTCCTGGTTTTC | 1779 |
| Increased Risk | CCACAGAAAATGATGCCCAGTGCTTAACAAGACCATACTACAG | 1780 |
| Thrombosis | TGACGTGGACATCATGAGAGACATCGCCTCTGGGCTAATAGG | |
| Arg485Lys | ACTACTTCTAATCTGTAAGAGCAGATCCCTGGACAG | |
| AGA-AAA | CTGTCCAGGGATCTGCTCTTACAGATTAGAAGTAGTCCTATTA | 1781 |
| | GCCCAGAGGCGATGTCTCTCATGATGTCCACGTCACTGTAGT | |
| | ATGGTCTTGTTAAGCACTGGGCATCATTTTCTGTGG | |
| | CATCATGAGAGACATCG | 1782 |
| | CGATGTCTCTCATGATG | 1783 |
| Increased Risk | ACATCGCCTCTGGGCTAATAGGACTACTTCTAATCTGTAAGAG | 1784 |
| Thrombosis | CAGATCCCTGGACAGGCGAGGAATACAGGTATTTTGTCCTTG | |
| Arg506Gln | AAGTAACCTTTCAGAAATTCTGAGAATTTCTTCTGG | |
| CGA-CAA | CCAGAAGAAATTCTCAGAATTTCTGAAAGGTTACTTCAAGGAC | 1785 |
| | AAAATACCTGTATTCCTCGCCTGTCCAGGGATCTGCTCTTACA | |
| | GATTAGAAGTAGTCCTATTAGCCCAGAGGCGATGT | |
| | GGACAGGCGAGGAATAC | 1786 |
| | GTATTCCTCGCCTGTCC | 1787 |
| Factor V Deficiency | GACATCGCCTCTGGGCTAATAGGACTACTTCTAATCTGTAAGA | 1788 |
| Arg506Term | GCAGATCCCTGGACAGGCGAGGAATACAGGTATTTTGTCCTT | |
| CGA-TGA | GAAGTAACCTTTCAGAAATTCTGAGAATTTCTTCTG | |
| | CAGAAGAAATTCTCAGAATTTCTGAAAGGTTACTTCAAGGACA | 1789 |
| | AAATACCTGTATTCCTCGCCTGTCCAGGGATCTGCTCTTACAG | |
| | ATTAGAAGTAGTCCTATTAGCCCAGAGGCGATGTC | |
| | TGGACAGGCGAGGAATA | 1790 |
| | TATTCCTCGCCTGTCCA | 1791 |
| Thrombosis | AGTGATGCTGACTATGATTACCAGAACAGACTGGCTGCAGCA | 1792 |
| Arg712Term | TTAGGAATCAGGTCATTCCGAAACTCATCATTGAATCAGGAAG | |
| CGA-TGA | AAGAAGAGTTCAATCTTACTGCCCTAGCTCTGGAGA | |
| | TCTCCAGAGCTAGGGCAGTAAGATTGAACTCTTCTTCTTCCTG | 1793 |
| | ATTCAATGATGAGTTTCGGAATGACCTGATTCCTAATGCTGCA | |
| | GCCAGTCTGTTCTGGTAATCATAGTCAGCATCACT | |
| | GGTCATTCCGAAACTCA | 1794 |
| | TGAGTTTCGGAATGACC | 1795 |
| Thrombosis | TCAGTCAGACAAACCTTTCCCCAGCCCTCGGTCAGATGCCCA | 1796 |
| His1299Arg | TTTCTCCAGACCTCAGCCATACAACCCTTTCTCTAGACTTCAG | |
| CAT-CGT | CCAGACAAACCTCTCTCCAGAACTCAGTCAAACAAA | |
| | TTTGTTTGACTGAGTTCTGGAGAGAGGTTTGTCTGGCTGAAGT | 1797 |
| | CTAGAGAAAGGGTTGTATGGCTGAGGTCTGGAGAAATGGGCA | |
| | TCTGACCGAGGGCTGGGGAAAGGTTTGTCTGACTGA | |
| | CCTCAGCCATACAACCC | 1798 |
| | GGGTTGTATGGCTGAGG | 1799 |

### EXAMPLE 14

### Hemophilia—Factor VIII Deficiency

The attached table discloses the correcting oligonucleotide base sequences for the Factor VIII oligonucleotides of the invention.

TABLE 16
Factor VIII Mutations and Genome-Correcting Oligos

| Clinical Phenotype & | | SEQ ID |
|---|---|---|
| Mutation | Correcting Oligos | NO: |
| Haemophilia A | AGCTCTCCACCTGCTTCTTTCTGTGCCTTTTGCGATTCTGCTT | 1800 |
| TyrSCys | TAGTGCCACCAGAAGATACTACCTGGGTGCAGTGGAACTGTC | |
| TAC-TGC | ATGGGACTATATGCAAAGTGATCTCGGTGAGCTGCC | |
| | GGCAGCTCACCGAGATCACTTTGCATATAGTCCCATGACAGT | 1801 |
| | TCCACTGCACCCAGGTAGTATCTTCTGGTGGCACTAAAGCAG | |
| | AATCGCAAAAGGCACAGAAAGAAGCAGGTGGAGAGCT | |
| | CAGAAGATACTACCTGG | 1802 |
| | CCAGGTAGTATCTTCTG | 1803 |
| Haemophilia A | CCACCTGCTTCTTTCTGTGCCTTTTGCGATTCTGCTTTAGTGC | 1804 |
| Leu7Arg | CACCAGAAGATACTACCTGGGTGCAGTGGAACTGTCATGGGA | |
| CTG-CGG | CTATATGCAAAGTGATCTCGGTGAGCTGCCTGTGGA | |
| | TCCACAGGCAGCTCACCGAGATCACTTTGCATATAGTCCCAT | 1805 |
| | GACAGTTCCACTGCACCCAGGTAGTATCTTCTGGTGGCACTA | |
| | AAGCAGAATCGCAAAAGGCACAGAAAGAAGCAGGTGG | |
| | ATACTACCTGGGTGCAG | 1806 |
| | CTGCACCCAee GGTAGTAT | 1807 |
| Haemophilia A | AGTCATGCAAATAGAGCTCTCCACCTGCTTCTTTCTGTGCCTT | 1808 |
| Ser(−1)Arg | TTGCGATTCTGCTTTAGIGCCACCAGAAGATACTACCTGGGT | |
| AGTg-AGG | GCAGTGGAACTGTCATGGGACTATATGCAAAGTGAT | |
| | ATCACTTTGCATATAGTCCCATGACAGTTCCACTGCACCCAGG | 1809 |
| | TAGTATCTTCTGGTGGCACTAAAGCAGAATCGCAAAAGGCAC | |
| | AGAAAGAAGCAGGTGGAGAGCTCTATTTGCATGACT | |
| | TGCTTTAGTGCCACCAG | 1810 |
| | CTGGTGGCACTAAAGCA | 1811 |
| Haemophilia A | CATTTGTAGCAATAAGTCATGCAAATAGAGCTCTCCACCTGCT | 1812 |
| Arg(−5)Term | TCTTTCTGTGCCTTTTGCGATTCTGCTTTAGTGCCACCAGAAG | |
| gCGA-TGA | ATACTACCTGGGTGCAGTGGAACTGTCATGGGACT | |
| | AGTCCCATGACAGTTCCACTGCACCCAGGTAGTATCTTCTGG | 1813 |
| | TGGCACTAAAGCAGAATCGCAAAAGGCACAGAAAGAAGCAG | |
| | GTGGAGAGCTCTATTTGCATGACTTATTGCTACAAATG | |
| | GCCTTTTGCGATTCTGC | 1814 |
| | GCAGAATCGCAAAAGGC | 1815 |
| Haemophilia A | TTCTGTGCCTTTTGCGATTCTGCTTTAGTGCCACCAGAAGATA | 1816 |
| Glu11Val | CTACCTGGGTGCAGTGGAACTGTCATGGGACTATATGCAAAG | |
| GAA-GTA | TGATCTCGGTGAGCTGCCTGTGGACGCAAGGTAAAG | |
| | CTTTACCTTGCGTCCACAGGCAGCTCACCGAGATCACTTTGC | 1817 |
| | ATATAGTCCCATGACAGTTCCACTGCACCCAGGTAGTATCTTC | |
| | TGGTGGCACTAAAGCAGAATCGCAAAAGGCACAGAA | |
| | TGCAGTGGAACTGTCAT | 1818 |
| | ATGACAGTTCCACTGCA | 1819 |
| Haemophilia A | CTTTTGCGATTCTGCTTTAGTGCCACCAGAAGATACTACCTGG | 1820 |
| Trp14Gly | GTGCAGTGGAACTGTCATGGGACTATATGCAAAGTGATCTCG | |
| aTGG-GGG | GTGAGCTGCCTGTGGACGCAAGGTAAAGGCATGTCC | |
| | GGACATGCCTTTACCTTGCGTCCACAGGCAGCTCACCGAGAT | 1821 |
| | CACTTTGCATATAGTCCCATGACAGTTCCACTGCACCCAGGT | |
| | AGTATCTTCTGGTGGCACTAAAGCAGAATCGCAAAAG | |
| | AACTGTCATGGGACTAT | 1822 |
| | ATAGTCCCATGACAGTT | 1823 |
| Haemophilia A | TTCACGCAGATTTCCTCCTAGAGTGCCAAAATCTTTTCCATTC | 1824 |
| Tyr46Term | AACACCTCAGTCGTGTACAAAAAGACTCTGTTTGTAGAATTCA | |
| TACa-TAA | CGGATCACCTTTTCAACATCGCTAAGCCAAGGCCA | |
| | TGGCCTTGGCTTAGCGATGTTGAAAAGGTGATCCGTGAATTC | 1825 |
| | TACAAACAGAGTCTTTTTGTACACGACTGAGGTGTTGAATGGA | |
| | AAAGATTTTGGCACTCTAGGAGGAAATCTGCGTGAA | |
| | GTCGTGTACAAAAAGAC | 1826 |
| | GTCTTTTTGTACACGAC | 1827 |
| Haemophilia A | ATCTTTTCCATTCAACACCTCAGTCGTGTACAAAAAGACTCTG | 1828 |
| Asp56Glu | TTTGTAGAATTCACGGATCACCTTTTCAACATCGCTAAGCCAA | |
| GATc-GAA | GGCCACCCTGGATGGGTAATGAAAACAATGTTGAA | |
| | TTCAACATTGTTTTCATTACCCATCCAGGGTGGCCTTGGCTTA | 1829 |
| | GCGATGTTGAAAAGGTGATCCGTGAATTCTACAAACAGAGTC | |
| | TTTTTGTACACGACTGAGGTGTTGAATGGAAAAGAT | |
| | TTCACGGATCACCTTTT | 1830 |
| | AAAAGGTGATCCGTGAA | 1831 |
| Haemophilia A | TTCTGGAGTACTATCCCCAAGTAACCTTTGGCGGACATCTCAT | 1832 |
| Gly73Val | TCTTACAGGTCTGCTAGGTCCTACCATCCAGGCTGAGGTTTAT | |
| GGT-GTT | GATACAGTGGTCATTACACTTAAGAACATGGCTTC | |
| | GAAGCCATGTTCTTAAGTGTAATGACCACTGTATCATAAACCT | 1833 |
| | CAGCCTGGATGGTAGGACCTAGCAGACCTGTAAGAATGAGAT | |
| | GTCCGCCAAAGGTTACTTGGGGATAGTACTCCAGAA | |
| | TCTGCTAGGTCCTACCA | 1834 |
| | TGGTAGGACCTAGCAGA | 1835 |
| Haemophilia A | CAAGTAACCTTTGGCGGACATCTCATTCTTACAGGTCTGCTAG | 1836 |
| Glu79Lys | GTCCTACCATCCAGGCTGAGGTTTATGATACAGTGGTCATTAC | |
| tGAG-AAG | ACTTAAGAACATGGCTTCCCATCCTGTCAGTCTTC | |
| | GAAGACTGACAGGATGGGAAGCCATGTTCTTAAGTGTAATGA | 1837 |
| | CCACTGTATCATAAACCTCAGCCTGGATGGTAGGACCTAGCA | |
| | GACCTGTAAGAATGAGATGTCCGCCAAAGGTTACTTG | |
| | TCCAGGCTGAGGTTTAT | 1838 |
| | ATAAACCTCAGCCTGGA | 1839 |
| Haemophilia A | TAACCTTTGGCGGACATCTCATTCTTACAGGTCTGCTAGGTCC | 1840 |
| ValSOAsp | TACCATCCAGGCTGAGGTTTATGATACAGTGGTCATTACACTT | |
| GTT-GAT | AAGAACATGGCTTCCCATCCTGTCAGTCTTCATGC | |
| | GCATGAAGACTGACAGGATGGGAAGCCATGTTCTTAAGTGTA | 1841 |
| | ATGACCACTGTATCATAAACCTCAGCCTGGATGGTAGGACCT | |
| | AGCAGACCTGTAAGAATGAGATGTCCGCCAAAGGTTA | |
| | GGCTGAGGTTTATGATA | 1842 |
| | TATCATAAACCTCAGCC | 1843 |
| Haemophilia A | TTGGCGGACATCTCATTCTTACAGGTCTGCTAGGTCCTACCAT | 1844 |
| Asp82Val | CCAGGCTGAGGTTTATGATACAGTGGTCATTACACTTAAGAA | |
| GAT-GTT | CATGGCTTCCCATCCTGTCAGTCTTCATGCTGTTGG | |
| | CCAACAGCATGAAGACTGACAGGATGGGAAGCCATGTTCTTA | 1845 |
| | AGTGTAATGACCACTGTATCATAAACCTCAGCCTGGATGGTA | |
| | GGACCTAGCAGACCTGTAAGAATGAGATGTCCGCCAA | |
| | GGTTTATGATACAGTGG | 1846 |
| | CCACTGTATCATAAACC | 1847 |
| Haemophilia A | TTGGCGGACATCTCATTCTTACAGGTCTGCTAGGTCCTACCAT | 1848 |
| Asp82Gly | CCAGGCTGAGGTTTATGATACAGTGGTCATTACACTTAAGAA | |
| GAT-GGT | CATGGCTTCCCATCCTGTCAGTCTTCATGCTGTTGG | |
| | CCAACAGCATGAAGACTGACAGGATGGGAAGCCATGTTCTTA | 1849 |
| | AGTGTAATGACCACTGTATCATAAACCTCAGCCTGGATGGTA | |
| | GGACCTAGCAGACCTGTAAGAATGAGATGTCCGCCAA | |
| | GGTTTATGATACAGTGG | 1850 |
| | CCACTGTATCATAAACC | 1851 |
| Haemophilia A | ATCTCATTCTTACAGGTCTGCTAGGTCCTACCATCCAGGCTGA | 1852 |
| Val85Asp | GGTTTATGATACAGTGGTCATTACACTTAAGAACATGGCTTCC | |
| GTC-GAC | CATCCTGTCAGTCTTCATGCTGTTGGTGTATCCTA | |
| | TAGGATACACCAACAGCATGAAGACTGACAGGATGGGAAGCC | 1853 |
| | ATGTTCTTAAGTGTAATGACCACTGTATCATAAACCTCAGCCT | |
| | GGATGGTAGGACCTAGCAGACCTGTAAGAATGAGAT | |
| | TACAGTGGTCATTACAC | 1854 |
| | GTGTAATGACCACTGTA | 1855 |
| Haemophilia A | CAGGTCTGCTAGGTCCTACCATCCAGGCTGAGGTTTATGATAT | 1856 |
| Lys89Thr | CAGTGGTCATTACACTTAAGAACATGGCTTCCCATCCTGTCAG | |
| AAG-ACG | TCTTCATGCTGTTGGTGTATCCTACTGGAAAGCTTC | |
| | GAAGCTTTCCAGTAGGATACACCAACAGCATGAAGACTGACA | 1857 |
| | GGATGGGAAGCCATGTTCTTAAGTGTAATGACCACTGTATCAT | |
| | AAACCTCAGCCTGGATGGTAGGACCTAGCAGACCTG | |
| | TACACTTAAGAACATGG | 1858 |
| | CCATGTTCTTAAGTGTA | 1859 |
| Haemophilia A | CTGCTAGGTCCTACCATCCAGGCTGAGGTTTATGATACAGTG | 1860 |
| Met91Val | GTCATTACACTTAAGAACATGGCTTCCCATCCTGTCAGTCTTC | |
| cATG-GTG | ATGCTGTTGGTGTATCCTACTGGAAAGCTTCTGAGG | |
| | CCTCAGAAGCTTTCCAGTAGGATACACCAACAGCATGAAGAC | 1861 |
| | TGACAGGATGGGAAGCCATGTTCTTAAGTGTAATGACCACTG | |
| | TATCATAAACCTCAGCCTGGATGGTAGGACCTAGCAG | |
| | TTAAGAACATGGCTTCC | 1862 |
| | GGAAGCCATGTTCTTAA | 1863 |
| Haemophilia A | CTACCATCCAGGCTGAGGTTTATGATACAGTGGTCATTACACT | 1864 |
| His94Arg | TAAGAACATGGCTTCCCATCCTGTCAGTCTTCATGCTGTTGGT | |
| CAT-CGT | GTATCCTACTGGAAAGCTTCTGAGGGTGAGTAAAA | |
| | TTTTACTCACCCTCAGAAGCTTTCCAGTAGGATACACCAACAG | 1865 |
| | CATGAAGACTGACAGGATGGGAAGCCATGTTCTTAAGTGTAA | |
| | TGACCACTGTATCATAAACCTCAGCCTGGATGGTAG | |
| | GGCTTCCCATCCTGTCA | 1866 |
| | TGACAGGATGGGAAGCC | 1867 |
| Haemophilia A | CCTACCATCCAGGCTGAGGTTTATGATACAGTGGTCATTACAC | 1868 |
| His94Tyr | TTAAGAACATGGCTTCCCATCCTGTCAGTCTTCATGCTGTTGG | |
| cCAT-TAT | TGTATCCTACTGGAAAGCTTCTGAGGGTGAGTAAA | |
| | TTTACTCACCCTCAGAAGCTTTCCAGTAGGATACACCAACAGC | 1869 |
| | ATGAAGACTGACAGGATGGGAAGCCATGTTCTTAAGTGTAAT | |
| | GACCACTGTATCATAAACCTCAGCCTGGATGGTAGG | |
| | TGGCTTCCCATCCTGTC | 1870 |
| | GACAGGATGGGAAGCCA | 1871 |
| Haemophilia A | CTGAGGTTTATGATACAGTGGTCATTACACTTAAGAACATGGC | 1872 |
| Leu98Arg | TTCCCATCCTGTCAGTCTTCATGCTGTTGGTGTATCCTACTGG | |
| CTT-CGT | AAAGCTTCTGAGGGTGAGTAAAATACCCTCCTATT | |
| | AATAGGAGGGTATTTTACTCACCCTCAGAAGCTTTCCAGTAG | 1873 |
| | GATACACCAACAGCATGAAGACTGACAGGATGGGAAGCCAT | |
| | GTTCTTAAGTGTAATGACCACTGTATCATAAACCTCAG | |
| | TGTCAGTCTTCATGCTG | 1874 |
| | CAGCATGAAGACTGACA | 1875 |
| Haemophilia A | GATACAGTGGTCATTACACTTAAGAACATGGCTTCCCATCCTG | 1876 |
| Gly102Ser | TCAGTCTTCATGCTGTTGGTGTATCCTACTGGAAAGCTTCTGA | |
| tGGT-AGT | GGGTGAGTAAAATACCCTCCTATTGTCCTGTCATT | |
| | AATGACAGGACAATAGGAGGGTATTTTACTCACCCTCAGAAG | 1877 |
| | CTTTCCAGTAGGATACACCAACAGCATGAAGACTGACAGGAT | |
| | GGGAAGCCATGTTCTTAAGTGTAATGACCACTGTATC | |
| | ATGCTGTTGGTGTATCC | 1878 |
| | GGATACACCAACAGCAT | 1879 |
| Haemophilia A | CTTTGAGTGTACAGTGGATATAGAAAGGACAATTTTATTTCTT | 1880 |
| Glu113Asp | CCTGCTATAGGAGCTGAATATGATGATCAGACCAGTCAAAGG | |
| GAAt-GAC | GAGAAAGAAGATGATAAAGTCTTCCCTGGTGGAAGC | |
| | GCTTCCACCAGGGAAGACTTTATCATCTTCTTTCTCCCTTTGA | 1881 |
| | CTGGTCTGATCATCATATTCAGCTCCTATAGCAGGAAGAAATA | |
| | AAATTGTCCTTTCTATATCCACTGTACACTCAAAG | |
| | GGAGCTGAATATGATGA | 1882 |
| | TCATCATATTCAGCTCC | 1883 |
| Haemophilia A | TTGAGTGTACAGTGGATATAGAAAGGACAATTTTATTTCTTCC | 1884 |
| Tyr114Cys | TGCTATAGGAGCTGAATATGATGATCAGACCAGTCAAAGGGA | |
| TAT-TGT | GAAAGAAGATGATAAAGTCTTCCCTGGTGGAAGCCA | |
| | TGGCTTCCACCAGGGAAGACTTTATCATCTTCTTTCTCCCTTT | 1885 |
| | GACTGGTCTGATCATCATATTCAGCTCCTATAGCAGGAAGAAA | |
| | TAAAATTGTCCTTTCTATATCCACTGTACACTCAA | |
| | AGCTGAATATGATGATC | 1886 |
| | GATCATCATATTCAGCT | 1887 |
| Haemophilia A | GTACAGTGGATATAGAAAGGACAATTTTATTTCTTCCTGCTAT | 1888 |
| Asp116Gly | AGGAGCTGAATATGATGATCAGACCAGTCAAAGGGAGAAAGA | |
| GAT-GGT | AGATGATAAAGTCTTCCCTGGTGGAAGCCATACATA | |
| | TATGTATGGCTTCCACCAGGGAAGACTTTATCATCTTCTTTCT | 1889 |
| | CCCTTTGACTGGTCTGATCATCATATTCAGCTCCTATAGCAGG | |
| | AAGAAATAAAATTGTCCTTTCTATATCCACTGTAC | |
| | ATATGATGATCAGACCA | 1890 |
| | TGGTCTGATCATCATAT | 1891 |
| Haemophilia A | ACAGTGGATATAGAAAGGACAATTTTATTTCTTCCTGCTATAG | 1892 |
| Gin117Term | GAGCTGAATATGATGATCAGACCAGTCAAAGGGAGAAAGAAG | |
| tCAG-TAG | ATGATAAAGTCTTCCCTGGTGGAAGCCATACATATG | |
| | CATATGTATGGCTTCCACCAGGGAAGACTTTATCATCTTCTTT | 1893 |
| | CTCCCTTTGACTGGTCTGATCATCATATTCAGCTCCTATAGCA | |
| | GGAAGAAATAAAATTGTCCTTTCTATATCCACTGT | |
| | ATGATGATCAGACCAGT | 1894 |
| | ACTGGTCTGATCATCAT | 1895 |
| Haemophilia A | TGGATATAGAAAGGACAATTTTATTTCTTCCTGCTATAGGAGC | 1896 |
| Thr118Ile | TGAATATGATGATCAGACCAGTCAAAGGGAGAAAGAAGATGA | |
| ACC-ATC | TAAAGTCTTCCCTGGTGGAAGCCATACATATGTCTG | |
| | CAGACATATGTATGGCTTCCACCAGGGAAGACTTTATCATCTT | 1897 |
| | CTTTCTCCCTTTGACTGGTCTGATCATCATATTCAGCTCCTATA | |
| | GCAGGAAGAAATAAAATTGTCCTTTCTATATCCA | |
| | TGATCAGACCAGTCAAA | 1898 |
| | TTTGACTGGTCTGATCA | 1899 |
| Haemophilia A | AGGACAATTTTATTTCTTCCTGCTATAGGAGCTGAATATGATG | 1900 |
| Glu122Term | ATCAGACCAGTCAAAGGGAGAAAGAAGATGATAAAGTCTTCC | |
| gGAG-TAG | CTGGTGGAAGCCATACATATGTCTGGCAGGTCCTGA | |
| | TCAGGACCTGCCAGACATATGTATGGCTTCCACCAGGGAAGA | 1901 |
| | CTTTATCATCTTCTTTCTCCCTTTGACTGGTCTGATCATCATAT | |
| | TCAGCTCCTATAGCAGGAAGAAATAAAATTGTCCT | |
| | GTCAAAGGGAGAAAGAA | 1902 |
| | TTCTTTCTCCCTTTGAC | 1903 |
| Haemophilia A | TTTCTTCCTGCTATAGGAGCTGAATATGATGATCAGACCAGTC | 1904 |
| Asp126His | AAAGGGAGAAAGAAGATGATAAAGTCTTCCCTGGTGGAAGCC | |
| tGAT-CAT | ATACATATGTCTGGCAGGTCCTGMAGAGAATGGTC | |
| | GACCATTCTCTTTCAGGACCTGCCAGACATATGTATGGCTTCC | 1905 |
| | ACCAGGGAAGACTTTATCATCTTCTTTCTCCCTTTGACTGGTC | |
| | TGATCATCATATTCAGCTCCTATAGCAGGAAGAAA | |
| | AAGAAGATGATAAAGTC | 1906 |
| | GACTTTATCATCTTCTT | 1907 |
| Haemophilia A | AGTCAAAGGGAGAAAGAAGATGATAAAGTCTTCCCTGGTGGA | 1908 |
| Gln139Term | AGCCATACATATGTCTGGCAGGTCCTGAAAGAGAATGGTCCA | |
| gCAG-TAG | ATGGCCTCTGACCCACTGTGCCTTACCTACTCATATC | |
| | GATATGAGTAGGTAAGGCACAGTGGGTCAGAGGCCATTGGA | 1909 |
| | CCATTCTCTTTCAGGACCTGCCAGACATATGTATGGCTTCCAC | |
| | CAGGGAAGACTTTATCATCTTCTTTCTCCCTTTGACT | |
| | ATGTCTGGCAGGTCCTG | 1910 |
| | CAGGACCTGCCAGACAT | 1911 |
| Haemophilia A | AAAGGGAGAAAGAAGATGATAAAGTCTTCCCTGGTGGAAGCC | 1912 |
| Val140Ala | ATACATATGTCTGGCAGGTCCTGAAAGAGAATGGTCCAATGG | |
| GTC-GCC | CCTCTGACCCACTGTGCCTTACCTACTCATATCTTTC | |
| | GAAAGATATGAGTAGGTAAGGCACAGTGGGTCAGAGGCCATT | 1913 |
| | GGACCATTCTCTTTCAGGACCTGCCAGACATATGTATGGCTT | |
| | CCACCAGGGAAGACTTTATCATCTTCTTTCTCCCTTT | |
| | CTGGCAGGTCCTGAAAG | 1914 |
| | CTTTCAGGACCTGCCAG | 1915 |
| Haemophilia A | AGATGATAAAGTCTTCCCTGGTGGAAGCCATACATATGTCTG | 1916 |
| Asn144Lys | GCAGGTCCTGAAAGAGAATGGTCCAATGGCCTCTGACCCACT | |
| AATg-AAA | GTGCCTTACCTACTCATATCTTTCTCATGTGGACCTG | |
| | CAGGTCCACATGAGAAAGATATGAGTAGGTAAGGCACAGTGG | 1917 |
| | GTCAGAGGCCATTGGACCATTCTCTTTCAGGACCTGCCAGAC | |
| | ATATGTATGGCTTCCACCAGGGAAGACTTTATCATCT | |
| | AAAGAGAATGGTCCAAT | 1918 |
| | ATTGGACCATTCTCTTT | 1919 |
| Haemophilia AG | TATGATAAAGTCTTCCCTGGTGGAAGCCATACATATGTCTGGC | 1920 |
| Gly145Asp | AGGTCCTGAAAGAGAATGGTCCAATGGCCTCTGACCCACTGT | |
| GGT-GAT | TGCCTTACCTACTCATATCTTTCTCATGTGGACCTGGT | |
| | ACCAGGTCCACATGAGAAAGATATGAGTAGGTAAGGCACAGT | 1921 |
| | GGGTCAGAGGCCATTGGACCATTCTCTTTCAGGACCTGCCAG | |
| | ACATATGTATGGCTTCCACCAGGGAAGACTTTATCAT | |
| | AGAGAATGGTCCAATGG | 1922 |
| | CCATTGGACCATTCTCT | 1923 |
| Haemophilia A | ATGATAAAGTCTTCCCTGGTGGAAGCCATACATATGTCTGGC | 1924 |
| Gly145Val | AGGTCCTGAAAGAGAATGGTCCAATGGCCTCTGACCCACTGT | |
| GGT-GTT | GCCTTACCTACTCATATCTTTCTCATGTGGACCTGGT | |
| | ACCAGGTCCACATGAGAAAGATATGAGTAGGTAAGGCACAGT | 1925 |
| | GGGTCAGAGGCCATTGGACCATTCTCTTTCAGGACCTGCCAG | |
| | ACATATGTATGGCTTCCACCAGGGAAGACTTTATCAT | |
| | AGAGAATGGTCCAATGG | 1926 |
| | CCATTGGACCATTCTCT | 1927 |
| Haemophilia A | GATAAAGTCTTCCCTGGTGGAAGCCATACATATGTCTGGCAG | 1928 |
| Pro146Ser | GTCCTGAAAGAGAATGGTCCAATGGCCTCTGACCCACTGTGC | |
| tCCA-TCA | CTTACCTACTCATATCTTTCTCATGTGGACCTGGTAA | |
| | TTACCAGGTCCACATGAGAAAGATATGAGTAGGTAAGGCACA | 1929 |
| | GTGGGTCAGAGGCCATTGGACCATTCTCTTTCAGGACCTGCC | |
| | AGACATATGTATGGCTTCCACCAGGGAAGACTTTATC | |
| | AGAATGGTCCAATGGCC | 1930 |
| | GGCCATTGGACCATTCT | 1931 |
| Haemophilia A | CCATACATATGTCTGGCAGGTCCTGAAAGAGAATGGTCCAAT | 1932 |
| Cys153Trp | GGCCTCTGACCCACTGTGCCTTACCTACTCATATCTTTCTCAT | |
| TGCc-TGG | GTGGACCTGGTAAAAGACTTGAATTCAGGCCTCATT | |
| | AATGAGGCCTGAATTCAAGTCTTTTACCAGGTCCACATGAGAA | 1933 |
| | AGATATGAGTAGGTAAGGCACAGTGGGTCAGAGGCCATTGG | |
| | ACCATTCTCTTTCAGGACCTGCCAGACATATGTATGG | |
| | CCACTGTGCCTTACCTA | 1934 |
| | TAGGTAAGGCACAGTGG | 1935 |
| Haemophilia A | TGTCTGGCAGGTCCTGAAAGAGAATGGTCCAATGGCCTCTGA | 1936 |
| Tyr156Term | CCCACTGTGCCTTACCTACTCATATCTTTCTCATGTGGACCTG | |
| TACt-TAA | GTAAAAGACTTGAATTCAGGCCTCATTGGAGCCCTA | |
| | TAGGGCTCCAATGAGGCCTGAATTCAAGTCTTTTACCAGGTC | 1937 |
| | CACATGAGAAAGATATGAGTAGGTAAGGCACAGTGGGTCAGA | |
| | GGCCATTGGACCATTCTCTTTCAGGACCTGCCAGACA | |
| | CTTACCTACTCATATCT | 1938 |
| | AGATATGAGTAGGTAAG | 1939 |
| Haemophilia A | GTCTGGCAGGTCCTGAAAGAGAATGGTCCAATGGCCTCTGAC | 1940 |
| Ser157Pro | CCACTGTGCCTTACCTACTCATATCTTTCTCATGTGGACCTGG | |
| cTCA-CCA | TAAAAGACTTGAATTCAGGCCTCATTGGAGCCCTAC | |
| | GTAGGGCTCCAATGAGGCCTGAATTCAAGTCTTTTACCAGGT | 1941 |
| | CCACATGAGAAAGATATGAGTAGGTAAGGCACAGTGGGTCAG | |
| | AGGCCATTGGACCATTCTCTTTCAGGACCTGCCAGAC | |
| | TTACCTACTCATATCTT | 1942 |
| | AAGATATGAGTAGGTAA | 1943 |
| Haemophilia A | GTCCTGAAAGAGAATGGTCCAATGGCCTCTGACCCACTGTGC | 1944 |
| Ser160Pro | CTTACCTACTCATATCTTTCTCATGTGGACCTGGTAAAAGACT | |
| tTCT-CCT | TGAATTCAGGCCTCATTGGAGCCCTACTAGTATGTA | |
| | TACATACTAGTAGGGCTCCAATGAGGCCTGAATTCAAGTCTTT | 1945 |
| | TACCAGGTCCACATGAGAAAGATATGAGTAGGTAAGGCACAG | |
| | TGGGTCAGAGGCCATTGGACCATTCTCTTTCAGGAC | |
| | CATATCTTTCTCATGTG | 1946 |
| | CACATGAGAAAGATATG | 1947 |
| Haemophilia A | AAAGAGAATGGTCCAATGGCCTCTGACCCACTGTGCCTTACC | 1948 |
| Val162Met | TACTCATATCTTTCTCATGTGGACCTGGTAAAAGACTTGAATT | |
| tGTG-ATG | CAGGCCTCATTGGAGCCCTACTAGTATGTAGAGAAG | |
| | CTTCTCTACATACTAGTAGGGCTCCAATGAGGCCTGAATTCAA | 1949 |
| | GTCTTTTACCAGGTCCACATGAGAAAGATATGAGTAGGTAAG | |
| | GCACAGTGGGTCAGAGGCCATTGGACCATTCTCTTT | |
| | TTTCTCATGTGGACCTG | 1950 |
| | CAGGTCCACATGAGAAA | 1951 |
| Haemophilia A | CAATGGCCTCTGACCCACTGTGCCTTACCTACTCATATCTTTC | 1952 |
| Lys166Thr | TCATGTGGACCTGGTAAAAGACTTGAATTCAGGCCTCATTGG | |
| AAA-ACA | AGCCCTACTAGTATGTAGAGAAGGTAAGTGTATGAA | |
| | TTCATACACTTACCTTCTCTACATACTAGTAGGGCTCCAATGA | 1953 |
| | GGCCTGAATTCAAGTCTTTTACCAGGTCCACATGAGAAAGATA | |
| | TGAGTAGGTAAGGCACAGTGGGTCAGAGGCCATTG | |
| | CCTGGTAAAAGACTTGA | 1954 |
| | TCAAGTCTTTTACCAGG | 1955 |
| Haemophilia A | ACCCACTGTGCCTTACCTACTCATATCTTTCTCATGTGGACCT | 1956 |
| Ser170Leu | GGTAAAAGACTTGAATTCAGGCCTCATTGGAGCCCTACTAGT | |
| TCA-TTA | ATGTAGAGAAGGTAAGTGTATGAAAGCGTAGGATTG | |
| | CAATCCTACGCTTTCATACACTTACCTTCTCTACATACTAGTAG | 1957 |
| | GGCTCCAATGAGGCCTGAATTCAAGTCTTTTACCAGGTCCAC | |
| | ATGAGAAAGATATGAGTAGGTAAGGCACAGTGGGT | |
| | CTTGAATTCAGGCCTCA | 1958 |
| | TGAGGCCTGAATTCAAG | 1959 |
| Haemophilia A | AATGTTCTCACTTCTTTTTCAGGGAGTCTGGCCAAGGAAAAGA | 1960 |
| Phe195Val | CACAGACCTTGCACAAATTTATACTACTTTTTGCTGTATTTGAT | |
| aTTT-GTT | GAAGGTTAGTGAGTCTTAATCTGAATTTTGGATT | |
| | AATCCAAAATTCAGATTAAGACTCACTAACCTTCATCAAATACA | 1961 |
| | GCAAAAAGTAGTATAAATTTGTGCAAGGTCTGTGTCTTTTCCT | |
| | TGGCCAGACTCCCTGAAAAAGAAGTGAGAACATT | |
| | TGCACAAATTTATACTA | 1962 |
| | TAGTATAAATTTGTGCA | 1963 |
| Haemophilia A | CTTCTTTTTCAGGGAGTCTGGCCAAGGAAAAGACACAGACCT | 1964 |
| Leu198His | TGCACAAATTTATACTACTTTTTGCTGTATTTGATGAAGGTTAG | |
| CTT-CAT | TGAGTCTTAATCTGAATTTTGGATTCCTGAAAGAA | |
| | TTCTTTCAGGAATCCAAAATTCAGATTAAGACTCACTAACCTTC | 1965 |
| | ATCAAATACAGCAAAAAGTAGTATAAATTTGTGCAAGGTCTGT | |
| | GTCTTTTCCTTGGCCAGACTCCCTGAAAAAGAAG | |
| | TATACTACTTTTTGCTG | 1966 |
| | CAGCAAAAAGTAGTATA | 1967 |
| Haemophilia A | TTTCAGGGAGTCTGGCCAAGGAAAAGACACAGACCTTGCACA | 1968 |
| Ala200Asp | AATTTATACTACTTTTTGCTGTATTTGATGAAGGTTAGTGAGTC | |
| GCT-GAT | TTAATCTGAATTTTGGATTCCTGAAAGAAATCCTC | |
| | GAGGATTTCTTTCAGGAATCCAAAATTCAGATTAAGACTCACT | 1969 |
| | AACCTTCATCAAATACAGCAAAAAGTAGTATAAATTTGTGCAA | |
| | GGTCTGTGTCTTTTCCTTGGCCAGACTCCCTGAAA | |
| | ACTTTTTGCTGTATTTG | 1970 |
| | CAAATACAGCAAAAAGT | 1971 |
| Haemophilia A | TTTTCAGGGAGTCTGGCCAAGGAAAAGACACAGACCTTGCAC | 1972 |
| Ala200Thr | AAATTTATACTACTTTTTGCTGTATTTGATGAAGGTTAGTGAGT | |
| tGCT-ACT | CTTAATCTGAATTTTGGATTCCTGAAAGAAATCCT | |
| | AGGATTTCTTTCAGGAATCCAAAATTCAGATTAAGACTCACTA | 1973 |
| | ACCTTCATCAAATACAGCAAAAAGTAGTATAAATTTGTGCAAG | |
| | GTCTGTGTCTTTTCCTTGGCCAGACTCCCTGAAAA | |
| | TACTTTTTGCTGTATTT | 1974 |
| | AAATACAGCAAAAAGTA | 1975 |
| Haemophilia A | AACTCCTTGATGCAGGATAGGGATGCTGCATCTGCTCGGGCC | 1976 |
| Val234Phe | TGGCCTAAAATGCACACAGTCAATGGTTATGTAAACAGGTCTC | |
| aGTC-TTC | TGCCAGGTATGTACACACCTGCTCAACAATCCTCAG | |
| | CTGAGGATTGTTGAGCAGGTGTGTACATACCTGGCAGAGACC | 1977 |
| | TGTTTACATAACCATTGACTGTGTGCATTTTAGGCCAGGCCCG | |
| | AGCAGATGCAGCATCCCTATCCTGCATCAAGGAGTT | |
| | TGCACACAGTCAATGGT | 1978 |
| | ACCATTGACTGTGTGCA | 1979 |
| Haemophilia A | ATTTCAGATTCTCTACTTCATAGCCATAGGTGTCTTATTCCTAC | 1980 |
| Gly247Glu | TTTACAGGTCTGATTGGATGCCACAGGAAATCAGTCTATTGG | |
| GGA-GAA | CATGTGATTGGAATGGGCACCACTCCTGAAGTGCA | |
| | TGCACTTCAGGAGTGGTGCCCATTCCAATCACATGCCAATAG | 1981 |
| | ACTGATTTCCTGTGGCATCCAATCAGACCTGTAAAGTAGGAAT | |
| | AAGACACCTATGGCTATGAAGTAGAGAATCTGAAAT | |
| | TCTGATTGGATGCCACA | 1982 |
| | TGTGGCATCCAATCAGA | 1983 |
| Haemophilia A | ATAGGTGTCTTATTCCTACTTTACAGGTCTGATTGGATGCCAC | 1984 |
| Trp255Cys | AGGAAATCAGTCTATTGGCATGTGATTGGAATGGGCACCACT | |
| TGGc-TGT | CCTGAAGTGCACTCAATATTCCTCGAAGGTCACACA | |
| | TGTGTGACCTTCGAGGAATATTGAGTGCACTTCAGGAGTGGT | 1985 |
| | GCCCATTCCAATCACATGCCAATAGACTGATTTCCTGTGGCAT | |
| | CCAATCAGACCTGTAAAGTAGGAATAAGACACCTAT | |
| | GTCTATTGGCATGTGAT | 1986 |
| | ATCACATGCCAATAGAC | 1987 |
| Haemophilia A | ATAGGTGTCTTATTCCTACTTTACAGGTCTGATTGGATGCCAC | 1988 |
| Trp255Term | AGGAAATCAGTCTATTGGCATGTGATTGGAATGGGCACCACT | |
| TGGc-TGA | CCTGAAGTGCACTCAATATTCCTCGAAGGTCACACA | |
| | TGTGTGACCTTCGAGGAATATTGAGTGCACTTCAGGAGTGGT | 1989 |
| | GCCCATTCCAATCACATGCCAATAGACTGATTTCCTGTGGCAT | |
| | CCAATCAGACCTGTAAAGTAGGAATAAGACACCTAT | |
| | GTCTATTGGCATGTGAT | 1990 |
| | ATCACATGCCAATAGAC | 1991 |
| Haemophilia A | AGGTGTCTTATTCCTACTTTACAGGTCTGATTGGATGCCACAG | 1992 |
| His256Leu | GAAATCAGTCTATTGGCATGTGATTGGAATGGGCACCACTCC | |
| CAT-CTT | TGAAGTGCACTCAATATTCCTCGAAGGTCACACATT | |
| | AATGTGTGACCTTCGAGGAATATTGAGTGCACTTCAGGAGTG | 1993 |
| | GTGCCCATTCCAATCACATGCCAATAGACTGATTTCCTGTGGC | |
| | ATCCAATCAGACCTGTAAAGTAGGAATAAGACACCT | |
| | CTATTGGCATGTGATTG | 1994 |
| | CAATCACATGCCAATAG | 1995 |
| Haemophilia A | TATTCCTACTTTACAGGTCTGATTGGATGCCACAGGAAATCAG | 1996 |
| Gly259Arg | TCTATTGGCATGTGATTGGAATGGGCACCACTCCTGAAGTGC | |
| tGGA-AGA | ACTCAATATTCCTCGAAGGTCACACATTTCTTGTGA | |
| | TCACAAGAAATGTGTGACCTTCGAGGAATATTGAGTGCACTTC | 1997 |
| | AGGAGTGGTGCCCATTCCAATCACATGCCAATAGACTGATTT | |
| | CCTGTGGCATCCAATCAGACCTGTAAAGTAGGAATA | |
| | ATGTGATTGGAATGGGC | 1998 |
| | GCCCATTCCAATCACAT | 1999 |
| Haemophilia A | TTGGATGCCACAGGAAATCAGTCTATTGGCATGTGATTGGAA | 2000 |
| Val266Gly | TGGGCACCACTCCTGAAGTGCACTCAATATTCCTCGAAGGTC | |
| GTG-GGG | ACACATTTCTTGTGAGGAACCATCGCCAGGCGTCCTT | |
| | AAGGACGCCTGGCGATGGTTCCTCACAAGAAATGTGTGACCT | 2001 |
| | TCGAGGAATATTGAGTGCACTTCAGGAGTGGTGCCCATTCCA | |
| | ATCACATGCCAATAGACTGATTTCCTGTGGCATCCAA | |
| | TCCTGAAGTGCACTCAA | 2002 |
| | TTGAGTGCACTTCAGGA | 2003 |
| Haemophilia A | CAGTCTATTGGCATGTGATTGGAATGGGCACCACTCCTGAAG | 2004 |
| Glu272Gly | TGCACTCAATATTCCTCGAAGGTCACACATTTCTTGTGAGGAA | |
| GAA-GGA | CCATCGCCAGGCGTCCTTGGAAATCTCGCCAATAAC | |
| | GTTATTGGCGAGATTTCCAAGGACGCCTGGCGATGGTTCCTC | 2005 |
| | ACAAGAAATGTGTGACCTTCGAGGAATATTGAGTGCACTTCA | |
| | GGAGTGGTGCCCATTCCAATCACATGCCAATAGACTG | |
| | ATTCCTCGAAGGTCACA | 2006 |
| | TGTGACCTTCGAGGAAT | 2007 |
| Haemophilia A | TCAGTCTATTGGCATGTGATTGGAATGGGCACCACTCCTGAA | 2008 |
| Glu272Lys | GTGCACTCAATATTCCTCGAAGGTCACACATTTCTTGTGAGGA | |
| cGAA-AAA | ACCATCGCCAGGCGTCCTTGGAAATCTCGCCAATAA | |
| | TTATTGGCGAGATTTCCAAGGACGCCTGGCGATGGTTCCTCA | 2009 |
| | CAAGAAATGTGTGACCTTCGAGGAATATTGAGTGCACTTCAG | |
| | GAGTGGTGCCCATTCCAATCACATGCCAATAGACTGA | |
| | TATTCCTCGAAGGTCAC | 2010 |
| | GTGACCTTCGAGGAATA | 2011 |
| Haemophilia A | GGCATGTGATTGGAATGGGCACCACTCCTGAAGTGCACTCAA | 2012 |
| Thr275Ile | TATTCCTCGAAGGTCACACATTTCTTGTGAGGAACCATCGCCA | |
| ACA-ATA | GGCGTCCTTGGAAATCTCGCCAATAACTTTCCTTAC | |
| | GTAAGGAAAGTTATTGGCGAGATTTCCAAGGACGCCTGGCGA | 2013 |
| | TGGTTCCTCACAAGAAATGTGTGACCTTCGAGGAATATTGAGT | |
| | GCACTTCAGGAGTGGTGCCCATTCCAATCACATGCC | |
| | AGGTCACACATTTCTTG | 2014 |
| | CAAGAAATGTGTGACCT | 2015 |
| Haemophilia A | TTGGAATGGGCACCACTCCTGAAGTGCACTCAATATTCCTCG | 2016 |
| Val278Ala | AAGGTCACACATTTCTTGTGAGGAACCATCGCCAGGCGTCCT | |
| GTG-GCG | TGGAAATCTCGCCAATAACTTTCCTTACTGCTCAAAC | |
| | GTTTGAGCAGTAAGGAAAGTTATTGGCGAGATTTCCAAGGAC | 2017 |
| | GCCTGGCGATGGTTCCTCACAAGAAATGTGTGACCTTCGAGG | |
| | AATATTGAGTGCACTTCAGGAGTGGTGCCCATTCCAA | |
| | ATTTCTTGTGAGGAACC | 2018 |
| | GGTTCCTCACAAGAAAT | 2019 |
| Haemophilia A | TGGGCACCACTCCTGAAGTGCACTCAATATTCCTCGAAGGTC | 2020 |
| Asn280Ile | ACACATTTCTTGTGAGGAACCATCGCCAGGCGTCCTTGGAAA | |
| AAC-ATC | TCTCGCCAATAACTTTCCTTACTGCTCAAACACTCTT | |
| | AAGAGTGTTTGAGCAGTAAGGAAAGTTATTGGCGAGATTTCC | 2021 |
| | AAGGACGCCTGGCGATGGTTCCTCACAAGAAATGTGTGACCT | |
| | TCGAGGAATATTGAGTGCACTTCAGGAGTGGTGCCCA | |
| | TGTGAGGAACCATCGCC | 2022 |
| | GGCGATGGTTCCTCACA | 2023 |
| Haemophilia A | ACCACTCCTGAAGTGCACTCAATATTCCTCGAAGGTCACACAT | 2024 |
| Arg282Cys | TTCTTGTGAGGAACCATCGCCAGGCGTCCTTGGAAATCTCGC | |
| tCGC-TGC | CAATAACTTTCCTTACTGCTCAAACACTCTTGATGG | |
| | CCATCAAGAGTGTTTGAGCAGTAAGGAAAGTTATTGGCGAGA | 2025 |
| | TTTCCAAGGACGCCTGGCGATGGTTCCTCACAAGAAATGTGT | |
| | GACCTTCGAGGAATATTGAGTGCACTTCAGGAGTGGT | |
| | GGAACCATCGCCAGGCG | 2026 |
| | CGCCTGGCGATGGTTCC | 2027 |
| Haemophilia A | CCACTCCTGAAGTGCACTCAATATTCCTCGAAGGTCACACATT | 2028 |
| Arg282His | TCTTGTGAGGAACCATCGCCAGGCGTCCTTGGAAATCTCGCC | |
| CGC-CAC | AATAACTTTCCTTACTGCTCAAACACTCTTGATGGA | |
| | TCCATCAAGAGTGTTTGAGCAGTAAGGAAAGTTATTGGCGAG | 2029 |
| | ATTTCCAAGGACGCCTGGCGATGGTTCCTCACAAGAAATGTG | |
| | TGACCTTCGAGGAATATTGAGTGCACTTCAGGAGTGG | |
| | GAACCATCGCCAGGCGT | 2030 |
| | ACGCCTGGCGATGGTTC | 2031 |
| Haemophilia A | CCACTCCTGAAGTGCACTCAATATTCCTCGAAGGTCACACATT | 2032 |
| Arg282Leu | TCTTGTGAGGAACCATCGCCAGGCGTCCTTGGAAATCTCGCC | |
| CGC-CTC | AATAACTTTCCTTACTGCTCAAACACTCTTGATGGA | |
| | TCCATCAAGAGTGTTTGAGCAGTAAGGAAAGTTATTGGCGAG | 2033 |
| | ATTTCCAAGGACGCCTGGCGATGGTTCCTCACAAGAAATGTG | |
| | TGACCTTCGAGGAATATTGAGTGCACTTCAGGAGTGG | |
| | GAACCATCGCCAGGCGT | 2034 |
| | ACGCCTGGCGATGGTTC | 2035 |
| Haemophilia A | CTGAAGTGCACTCAATATTCCTCGAAGGTCACACATTTCTTGT | 2036 |
| Ala284Glu | GAGGAACCATCGCCAGGCGTCCTTGGAAATCTCGCCAATAAC | |
| GCG-GAG | TTTCCTTACTGCTCAAACACTCTTGATGGACCTTGG | |
| | CCAAGGTCCATCAAGAGTGTTTGAGCAGTAAGGAAAGTTATT | 2037 |
| | GGCGAGATTTCCAAGGACGCCTGGCGATGGTTCCTCACAAG | |
| | AAATGTGTGACCTTCGAGGAATATTGAGTGCACTTCAG | |
| | TCGCCAGGCGTCCTTGG | 2038 |
| | CCAAGGACGCCTGGCGA | 2039 |
| Haemophilia A | CCTGAAGTGCACTCAATATTCCTCGAAGGTCACACATTTCTTG | 2040 |
| Ala284Pro | TGAGGAACCATCGCCAGGCGTCCTTGGAAATCTCGCCAATAA | |
| gGCG-CCG | CTTTCCTTACTGCTCAAACACTCTTGATGGACCTTG | |
| | CAAGGTCCATCAAGAGTGTTTGAGCAGTAAGGAAAGTTATTG | 2041 |
| | GCGAGATTTCCAAGGACGCCTGGCGATGGTTCCTCACAAGAA | |
| | ATGTGTGACCTTCGAGGAATATTGAGTGCACTTCAGG | |
| | ATCGCCAGGCGTCCTTG | 2042 |
| | CAAGGACGCCTGGCGAT | 2043 |
| Haemophilia A | TATTCCTCGAAGGTCACACATTTCTTGTGAGGAACCATCGCCA | 2044 |
| Ser289Leu | GGCGTCCTTGGAAATCTCGCCAATAACTTTCCTTACTGCTCAA | |
| TCG-TTG | ACACTCTTGATGGACCTTGGACAGTTTCTACTGTT | |
| | AACAGTAGAAACTGTCCAAGGTCCATCAAGAGTGTTTGAGCA | 2045 |
| | GTAAGGAAAGTTATTGGCGAGATTTCCAAGGACGCCTGGCGA | |
| | TGGTTCCTCACAAGAAATGTGTGACCTTCGAGGAATA | |
| | GGAAATCTCGCCAATAA | 2046 |
| | TTATTGGCGAGATTTCC | 2047 |
| Haemophilia A | GTCACACATTTCTTGTGAGGAACCATCGCCAGGCGTCCTTGG | 2048 |
| Phe293Ser | AAATCTCGCCAATAACTTTCCTTACTGCTCAAACACTCTTGATT | |
| TTC-TCC | GGACCTTGGACAGTTTCTACTGTTTTGTCATATCTC | |
| | GAGATATGACAAAACAGTAGAAACTGTCCAAGGTCCATCAAG | 2049 |
| | AGTGTTTGAGCAGTAAGGAAAGTTATTGGCGAGATTTCCAAG | |
| | GACGCCTGGCGATGGTTCCTCACAAGAAATGTGTGAC | |
| | AATAACTTTCCTTACTG | 2050 |
| | CAGTAAGGAAAGTTATT | 2051 |
| Haemophilia A | ACATTTCTTGTGAGGAACCATCGCCAGGCGTCCTTGGAAATC | 2052 |
| Thr295Ala | TCGCCAATAACTTTCCTTACTGCTCAAACACTCTTGATGGACC | |
| tACT-GCT | TTGGACAGTTTCTACTGTTTTGTCATATCTCTTCCC | |
| | GGGAAGAGATATGACAAAACAGTAGAAACTGTCCAAGGTCCA | 2053 |
| | TCAAGAGTGTTTGAGCAGTAAGGAAAGTTATTGGCGAGATTT | |
| | CCAAGGACGCCTGGCGATGGTTCCTCACAAGAAATGT | |
| | CTTTCCTTACTGCTCAA | 2054 |
| | TTGAGCAGTAAGGAAAG | 2055 |
| Haemophilia A | CATTTCTTGTGAGGAACCATCGCCAGGCGTCCTTGGAAATCT | 2056 |
| Thr295Ile | CGCCAATAACTTTCCTTACTGCTCAAACACTCTTGATGGACCT | |
| ACT-ATT | TGGACAGTTTCTACTGTTTTGTCATATCTCTTCCCA | |
| | TGGGAAGAGATATGACAAAACAGTAGAAACTGTCCAAGGTCC | 2057 |
| | ATCAAGAGTGTTTGAGCAGTAAGGAAAGTTATTGGCGAGATTT | |
| | CCAAGGACGCCTGGCGATGGTTCCTCACAAGAAATG | |
| | TTTCCTTACTGCTCAAA | 2058 |
| | TTTGAGCAGTAAGGAAA | 2059 |
| Haemophilia A | TTCTTGTGAGGAACCATCGCCAGGCGTCCTTGGAAATCTCGC | 2060 |
| Ala296Val | CAATAACTTTCCTTACTGCTCAAACACTCTTGATGGACCTTGG | |
| GCT-GTT | ACAGTTTCTACTGTTTTGTCATATCTCTTCCCACCA | |
| | TGGTGGGAAGAGATATGACAAAACAGTAGAAACTGTCCAAGG | 2061 |
| | TCCATCAAGAGTGTTTGAGCAGTAAGGAAAGTTATTGGCGAG | |
| | ATTTCCAAGGACGCCTGGCGATGGTTCCTCACAAGAA | |
| | CCTTACTGCTCAAACAC | 2062 |
| | GTGTTTGAGCAGTAAGG | 2063 |
| Haemophilia A | TCTCGCCAATAACTTTCCTTACTGCTCAAACACTCTTGATGGA | 2064 |
| Leu308Pro | CCTTGGACAGTTTCTACTGTTTTGTCATATCTCTTCCCACCAA | |
| CTG-CCG | CATGGTAATATCTTGGATCTTTAAAATGAATATTA | |
| | TAATATTCATTTTAAAGATCCAAGATATTACCATGTTGGTGGG | 2065 |
| | AAGAGATATGACAAAACAGTAGAAACTGTCCAAGGTCCATCA | |
| | AGAGTGTTTGAGCAGTAAGGAAAGTTATTGGCGAGA | |
| | GTTTCTACTGTTTTGTC | 2066 |
| | GACAAAACAGTAGAAAC | 2067 |
| Haemophilia A | ACAGCCTAATATAGCAAGACACTCTGACATTGTTTGGTTTGTC | 2068 |
| Glu321Lys | TGACTCCAGATGGCATGGAAGCTTATGTCAAAGTAGACAGCT | |
| gGAA-AAA | GTCCAGAGGAACCCCAACTACGAATGAAAAATAATG | |
| | CATTATTTTTCATTCGTAGTTGGGGTTCCTCTGGACAGCTGTC | 2069 |
| | TACTTTGACATAAGCTTCCATGCCATCTGGAGTCAGACAAACC | |
| | AAACAATGTCAGAGTGTCTTGCTATATTAGGCTGT | |
| | ATGGCATGGAAGCTTAT | 2070 |
| | ATAAGCTTCCATGCCAT | 2071 |
| Haemophilia A | ATATAGCAAGACACTCTGACATTGTTTGGTTTGTCTGACTCCA | 2072 |
| Tyr323Term | GATGGCATGGAAGCTTATGTCAAAGTAGACAGCTGTCCAGAG | |
| TATg-TAA | GAACCCCAACTACGAATGAAAAATAATGAAGAAGCG | |
| | CGCTTCTTCATTATTTTTCATTCGTAGTTGGGGTTCCTCTGGA | 2073 |
| | CAGCTGTCTACTTTGACATAAGCTTCCATGCCATCTGGAGTCA | |
| | GACAAACCAAACAATGTCAGAGTGTCTTGCTATAT | |
| | GAAGCTTATGTCAAAGT | 2074 |
| | ACTTTGACATAAGCTTC | 2075 |
| Haemophilia A | AAGACACTCTGACATTGTTTGGTTTGTCTGACTCCAGATGGCA | 2076 |
| Val326Leu | TGGAAGCTTATGTCAAAGTAGACAGCTGTCCAGAGGAACCCC | |
| aGTA-CTA | AACTACGAATGAAAAATAATGAAGAAGCGGAAGACT | |
| | AGTCTTCCGCTTCTTCATTATTTTTCATTCGTAGTTGGGGTTCC | 2077 |
| | TCTGGACAGCTGTCTACTTTGACATAAGCTTCCATGCCATCTG | |
| | GAGTCAGACAAACCAAACAATGTCAGAGTGTCTT | |
| | ATGTCAAAGTAGACAGC | 2078 |
| | GCTGTCTACTTTGACAT | 2079 |
| Haemophilia A | TGACATTGTTTGGTTTGTCTGACTCCAGATGGCATGGAAGCTT | 2080 |
| Cys329Arg | ATGTCAAAGTAGACAGCTGTCCAGAGGAACCCCAACTACGAA | |
| cTGT-CGT | TGAAAAATAATGAAGAAGCGGAAGACTATGATGATG | |
| | CATCATCATAGTCTTCCGCTTCTTCATTATTTTTCATTCGTAGT | 2081 |
| | TGGGGTTCCTCTGGACAGCTGTCTACTTTGACATAAGCTTCC | |
| | ATGCCATCTGGAGTCAGACAAACCAAACAATGTCA | |
| | TAGACAGCIGTCCAGAG | 2082 |
| | CTCTGGACAGCTGTCTA | 2083 |
| Haemophilia A | GACATTGTTTGGTTTGTCTGACTCCAGATGGCATGGAAGCTTA | 2084 |
| Cys329Tyr | TGTCAAAGTAGACAGCTGTCCAGAGGAACCCCAACTACGAAT | |
| TGT-TAT | GAAAAATAATGAAGAAGCGGAAGACTATGATGATGA | |
| | TCATCATCATAGTCTTCCGCTTCTTCATTATTTTTCATTCGTAG | 2085 |
| | TTGGGGTTCCTCTGGACAGCTGTCTACTTTGACATAAGCTTCC | |
| | ATGCCATCTGGAGTCAGACAAACCAAACAATGTC | |
| | AGACAGCTGTCCAGAGG | 2086 |
| | CCTCTGGACAGCTGTCT | 2087 |
| Haemophilia A | ACTCCAGATGGCATGGAAGCTTATGTCAAAGTAGACAGCTGT | 2088 |
| Arg336Term | CCAGAGGAACCCCAACTACGAATGAAAAATAATGAAGAAGCG | |
| aCGA-TGA | GAAGACTATGATGATGATCTTACTGATTCTGAAATGG | |
| | CCATTTCAGAATCAGTAAGATCATCATCATAGTCTTCCGCTTC | 2089 |
| | TTCATTATTTTTCATTCGTAGTTGGGGTTCCTCTGGACAGCTG | |
| | TCTACTTTGACATAAGCTTCCATGCCATCTGGAGT | |
| | CCCAACTACGAATGAAA | 2090 |
| | TTTCATTCGTAGTTGGG | 2091 |
| Haemophilia A | GATTCTGAAATGGATGTGGTCAGGTTTGATGATGACAACTCTC | 2092 |
| Arg372Cys | CTTCCTTTATCCAAATTCGCTCAGTTGCCAAGAAGCATCCTAA | |
| tCGC-TGC | AACTTGGGTACATTACATTGCTGCTGAAGAGGAGG | |
| | CCTCCTCTTCAGCAGCAATGTAATGTACCCAAGTTTTAGGATG | 2093 |
| | CTTCTTGGCAACTGAGCGAATTTGGATAAAGGAAGGAGAGTT | |
| | GTCATCATCAAACCTGACCACATCCATTTCAGAATC | |
| | TCCAAATTCGCTCAGTT | 2094 |
| | AACTGAGCGAATTTGGA | 2095 |
| Haemophilia A | ATTCTGAAATGGATGTGGTCAGGTTTGATGATGACAACTCTCC | 2096 |
| Arg372His | TTCCTTTATCCAAATTCGCTCAGTTGCCAAGAAGCATCCTAAA | |
| CGC-CAC | ACTTGGGTACATTACATTGCTGCTGAAGAGGAGGA | |
| | TCCTCCTCTTCAGCAGCAATGTAATGTACCCAAGTTTTAGGAT | 2097 |
| | GCTTCTTGGCAACTGAGCGAATTTGGATAAAGGAAGGAGAGT | |
| | TGTCATCATCAAACCTGACCACATCCATTTCAGAAT | |
| | CCAAATTCGCTCAGTTG | 2098 |
| | CAACTGAGCGAATTTGG | 2099 |
| Haemophilia A | CTGAAATGGATGTGGTCAGGTTTGATGATGACAACTCTCCTTC | 2100 |
| Ser373Leu | CTTTATCCAAATTCGCTCAGTTGCCAAGAAGCATCCTAAAACT | |
| TCA-TTA | TGGGTACATTACATTGCTGCTGAAGAGGAGGACTG | |
| | CAGTCCTCCTCTTCAGCAGCAATGTAATGTACCCAAGTTTTAG | 2101 |
| | GATGCTTCTTGGCAACTGAGCGAATTTGGATAAAGGAAGGAG | |
| | AGTTGTCATCATCAAACCTGACCACATCCATTTCAG | |
| | AATTCGCTCAGTTGCCA | 2102 |
| | TGGCAACTGAGCGAATT | 2103 |
| Haemophilia A | TCTGAAATGGATGTGGTCAGGTTTGATGATGACAACTCTCCTT | 2104 |
| Ser373Pro | CCTTTATCCAAATTCGCTCAGTTGCCAAGAAGCATCCTAAAAC | |
| cTCA-CCA | TTGGGTACATTACATTGCTGCTGAAGAGGAGGACT | |
| | AGTCCTCCTCTTCAGCAGCAATGTAATGTACCCAAGTTTTAGG | 2105 |
| | ATGCTTCTTGGCAACTGAGCGAATTTGGATTAAAGGAAGGAGA | |
| | GTTGTCATCATCAAACCTGACCACATCCATTTCAGA | |
| | AAATTCGCTCAGTTGCC | 2106 |
| | GGCAACTGAGCGAATTT | 2107 |
| Haemophilia A | CTGAAATGGATGTGGTCAGGTTTGATGATGACAACTCTCCTTC | 2108 |
| Ser373Term | CTTTATCCAAATTCGCTCAGTTGCCAAGAAGCATCCTAAAACT | |
| TCA-TAA | TGGGTACATTACATTGCTGCTGAAGAGGAGGACTG | |
| | CAGTCCTCCTCTTCAGCAGCAATGTAATGTACCCAAGTTTTAG | 2109 |
| | GATGCTTCTTGGCAACTGAGCGAATTTGGATAAAGGAAGGAG | |
| | AGTTGTCATCATCAAACCTGACCACATCCATTTCAG | |
| | AATTCGCTCAGTTGCCA | 2110 |
| | TGGCAACTGAGCGAATT | 2111 |
| Haemophilia A | CCTTCCTTTATCCAAATTCGCTCAGTTGCCAAGAAGCATCCTA | 2112 |
| Ile386Phe | AAACTTGGGTACATTACATTGCTGCTGAAGAGGAGGACTGGG | |
| cATT-TTT | ACTATGCTCCCTTAGTCCTCGCCCCCGATGACAGGT | |
| | ACCTGTCATCGGGGGCGAGGACTAAGGGAGCATAGTCCCAG | 2113 |
| | TCCTCCTCTTCAGCAGCAATGTAATGTACCCAAGTTTTAGGAT | |
| | GCTTCTTGGCAACTGAGCGAATTTGGATAAAGGAAGG | |
| | TACATTACATTGCTGCT | 2114 |
| | AGCAGCAATGTAATGTA | 2115 |
| Haemophilia A | CTTCCTTTATCCAAATTCGCTCAGTTGCCAAGAAGCATCCTAA | 2116 |
| Ile386Ser | AACTTGGGTACATTACATTGCTGCTGAAGAGGAGGACTGGGA | |
| ATT-AGT | CTATGCTCCCTTAGTCCTCGCCCCCGATGACAGGTA | |
| | TACCTGTCATCGGGGGCGAGGACTAAGGGAGCATAGTCCCA | 2117 |
| | GTCCTCCTCTTCAGCAGCAATGTAATGTACCCAAGTTTTAGGA | |
| | TGCTTCTTGGCAACTGAGCGAATTTGGATAAAGGAAG | |
| | ACATTACATTGCTGCTG | 2118 |
| | CAGCAGCAATGTAATGT | 2119 |
| Haemophilia A | AAATTCGCTCAGTTGCCAAGAAGCATCCTAAAACTTGGGTACA | 2120 |
| Glu390Gly | TTACATTGCTGCTGAAGAGGAGGACTGGGACTATGCTCCCTT | |
| GAG-GGG | AGTCCTCGCCCCCGATGACAGGTAAGCACTTTTTGA | |
| | TCAAAAAGTGCTTACCTGTCATCGGGGGCGAGGACTAAGGGA | 2121 |
| | GCATAGTCCCAGTCCTCCTCTTCAGCAGCAATGTAATGTACC | |
| | CAAGTTTTAGGATGCTTCTTGGCAACTGAGCGAATTT | |
| | TGCTGAAGAGGAGGACT | 2122 |
| | AGTCCTCCTCTTCAGCA | 2123 |
| Haemophilia A | TCAGTTGCCAAGAAGCATCCTAAAACTTGGGTACATTACATTG | 2124 |
| Trp393Gly | CTGCTGAAGAGGAGGACTGGGACTATGCTCCCTTAGTCCTCG | |
| cTGG-GGG | CCCCCGATGACAGGTAAGCACTTTTTGACTATTGGT | |
| | ACCAATAGTCAAAAAGTGCTTACCTGTCATCGGGGGCGAGGA | 2125 |
| | CTAAGGGAGCATAGTCCCAGTCCTCCTCTTCAGCAGCAATGT | |
| | AATGTACCCAAGTTTTAGGATGCTTCTTGGCAACTGA | |
| | AGGAGGACTGGGACTAT | 2126 |
| | ATAGTCCCAGTCCTCCT | 2127 |
| Haemophilia A | GCCTACCTAGAATTTTTCTTCCCAACCTCTCATCTTTTTTTCTC | 2128 |
| Lys408Ile | TTATACAGAAGTTATAAAAGTCAATATTTGAACAATGGCCCTC | |
| AAA-ATA | AGCGGATTGGTAGGAAGTACAAAAAAGTCCGATT | |
| | AATCGGACTTTTTTGTACTTCCTACCAATCCGCTGAGGGCCAT | 2129 |
| | TGTTCAAATATTGACTTTTATAACTTCTGTATAAGAGAAAAAAA | |
| | GATGAGAGGTTGGGAAGAAAAATTCTAGGTAGGC | |
| | AAGTTATAAAAGTCAAT | 2130 |
| | ATTGACTTTTATAACTT | 2131 |
| Haemophilia A | TTTTCTTCCCAACCTCTCATCTTTTTTTCTCTTATACAGAAGTT | 2132 |
| Leu412Phe | ATAAAAGTCAATATTTGAACAATGGCCCTCAGCGGATTGGTAG | |
| TTGa-TTT | GAAGTACAAAAAAGTCCGATTTATGGCATACACA | |
| | TGTGTATGCCATAAATCGGACTTTTTTGTACTTCCTACCAATC | 2133 |
| | CGCTGAGGGCCATTGTTCAAATATTGACTTTTATAACTTCTGT | |
| | ATAAGAGAAAAAAAGATGAGAGGTTGGGAAGAAAA | |
| | CAATATTTGAACAATGG | 2134 |
| | CCATTGTTCAAATATTG | 2135 |
| Haemophilia A | TCATCTTTTTTTCTCTTATACAGAAGTTATAAAAGTCAATATTTG | 2136 |
| Arg418Trp | AACAATGGCCCTCAGCGGATTGGTAGGAAGTACAAAAAAGTC | |
| gCGG-TGG | CGATTTATGGCATACACAGATGAAACCTTTAAGA | |
| | TCTTAAAGGTTTCATCTGTGTATGCCATAAATCGGACTTTTTTG | 2137 |
| | TACTTCCTACCAATCCGCTGAGGGCCATTGTTCAAATATTGAC | |
| | TTTTATAACTTCTGTATAAGAGAAAAAAAGATGA | |
| | GCCCTCAGCGGATTGGT | 2138 |
| | ACCAATCCGCTGAGGGC | 2139 |
| Haemophilia A | TTTTTCTCTTATACAGAAGTTATAAAAGTCAATATTTGAACAAT | 2140 |
| Gly420Val | GGCCCTCAGCGGATTGGTAGGAAGTACAAAAAAGTCCGATTT | |
| GGT-GTT | ATGGCATACACAGATGAAACCTTTAAGACTCGTGA | |
| | TCACGAGTCTTAAAGGTTTCATCTGTGTATGCCATAAATCGGA | 2141 |
| | CTTTTTTGTACTTCCTACCAATCCGCTGAGGGCCATTGTTCAA | |
| | ATATTGACTTTTATAACTTCTGTATAAGAGAAAAA | |
| | GCGGATTGGTAGGAAGT | 2142 |
| | ACTTCCTACCAATCCGC | 2143 |
| Haemophilia A | GAAGTTATAAAAGTCAATATTTGAACAATGGCCCTCAGCGGAT | 2144 |
| Lys425Arg | TGGTAGGAAGTACAAAAAAGTCCGATTTATGGCATACACAGA | |
| AAA-AGA | TGAAACCTTTAAGACTCGTGAAGCTATTCAGCATGA | |
| | TCATGCTGAATAGCTTCACGAGTCTTAAAGGTTTCATCTGTGT | 2145 |
| | ATGCCATAAATCGGACTTTTTTGTACTTCCTACCAATCCGCTG | |
| | AGGGCCATTGTTCAAATATTGACTTTTATAACTTC | |
| | GTACAAAAAAGTCCGAT | 2146 |
| | ATCGGACTTTTTTGTAC | 2147 |
| Haemophilia A | TATAAAAGTCAATATTTGAACAATGGCCCTCAGCGGATTGGTA | 2148 |
| Arg427Term | GGAAGTACAAAAAAGTCCGATTTATGGCATACACAGATGAAA | |
| cCGA-TGA | CCTTTAAGACTCGTGAAGCTATTCAGCATGAATCAG | |
| | CTGATTCATGCTGAATAGCTTCACGAGTCTTAAAGGTTTCATC | 2149 |
| | TGTGTATGCCATAAATCGGACTTTTTTGTACTTCCTACCAATC | |
| | CGCTGAGGGCCATTGTTCAAATATTGACTTTTATA | |
| | AAAAAGTCCGATTTATG | 2150 |
| | CATAAATCGGACTTTTT | 2151 |
| Haemophilia A | TATTTGAACAATGGCCCTCAGCGGATTGGTAGGAAGTACAAA | 2152 |
| Tyr431Asn | AAAGTCCGATTTATGGCATACACAGATGAAACCTTTAAGACTC | |
| aTAC-AAC | GTGAAGCTATTCAGCATGAATCAGGAATCTTGGGAC | |
| | GTCCCAAGATTCCTGATTCATGCTGAATAGCTTCACGAGTCTT | 2153 |
| | AAAGGTTTCATCTGTGTATGCCATAAATCGGACTTTTTTGTAC | |
| | TTCCTACCAATCCGCTGAGGGCCATTGTTCAAATA | |
| | TTATGGCATACACAGAT | 2154 |
| | ATCTGTGTATGCCATAA | 2155 |
| Haemophilia A | GCCCTCAGCGGATTGGTAGGAAGTACAAAAAAGTCCGATTTA | 2156 |
| Thr435Ile | TGGCATACACAGATGAAACCTTTAAGACTCGTGAAGCTATTCA | |
| ACC-ATC | GCATGAATCAGGAATCTTGGGACCTTTACTTTATGG | |
| | CCATAAAGTAAAGGTCCCAAGATTCCTGATTCATGCTGAATAG | 2157 |
| | CTTCACGAGTCTTAAAGGTTTCATCTGTGTATGCCATAAATCG | |
| | GACTTTTTTGTACTTCCTACCAATCCGCTGAGGGC | |
| | AGATGAAACCTTTAAGA | 2158 |
| | TCTTAAAGGTTTCATCT | 2159 |
| Haemophilia A | ACACAGATGAAACCTTTAAGACTCGTGAAGCTATTCAGCATGA | 2160 |
| Pro451Leu | ATCAGGAATCTTGGGACCTTTACTTTATGGGGAAGTTGGAGA | |
| CCT-CTT | CACACTGTTGGTAAGTTGAAGAAAAGATTTAAGGTC | |
| | GACCTTAAATCTTTTCTTCAACTTACCAACAGTGTGTCTCCAA | 2161 |
| | CTTCCCCATAAAGTAAAGGTCCCAAGATTCCTGATTCATGCTG | |
| | AATAGCTTCACGAGTCTTAAAGGTTTCATCTGTGT | |
| | CTTGGGACCTTTACTTT | 2162 |
| | AAAGTAAAGGTCCCAAG | 2163 |
| Haemophilia A | TACACAGATGAAACCTTTAAGACTCGTGAAGCTATTCAGCATG | 2164 |
| Pro451Thr | AATCAGGAATCTTGGGACCTTTACTTTATGGGGAAGTTGGAG | |
| aCCT-ACT | ACACACTGTTGGTAAGTTGAAGAAAAGATTTAAGGT | |
| | ACCTTAAATCTTTTCTTCAACTTACCAACAGTGTGTCTCCAACT | 2165 |
| | TCCCCATAAAGTAAAGGTCCCAAGATTCCTGATTCATGCTGAA | |
| | TAGCTTCACGAGTCTTAAAGGTTTCATCTGTGTA | |
| | TCTTGGGACCTTTACTT | 2166 |
| | AAGTAAAGGTCCCAAGA | 2167 |
| Haemophilia A | ACCTTTAAGACTCGTGAAGCTATTCAGCATGAATCAGGAATCT | 2168 |
| Gly455Arg | TGGGACCTTTACTTTATGGGGAAGTTGGAGACACACTGTTGG | |
| tGGG-AGG | TAAGTTGAAGAAAAGATTTAAGGTCAGGTAAGAAGA | |
| | TCTTCTTACCTGACCTTAAATCTTTTCTTCAACTTACCAACAGT | 2169 |
| | GTGTCTCCAACTTCCCCATAAAGTAAAGGTCCCAAGATTCCTG | |
| | ATTCATGCTGAATAGCTTCACGAGTCTTAAAGGT | |
| | TACTTTATGGGGAAGTT | 2170 |
| | AACTTCCCCATAAAGTA | 2171 |
| Haemophilia A | CCTTTAAGACTCGTGAAGCTATTCAGCATGAATCAGGAATCTT | 2172 |
| Gly455Glu | GGGACCTTTACTTTATGGGGAAGTTGGAGACACACTGTTGGT | |
| GGG-GAG | AAGTTGAAGAAAAGATTTAAGGTCAGGTAAGAAGAA | |
| | TTCTTCTTACCTGACCTTAAATCTTTTCTTCAACTTACCAACAG | 2173 |
| | TGTGTCTCCAACTTCCCCATAAAGTAAAGGTCCCAAGATTCCT | |
| | GATTCATGCTGAATAGCTTCACGAGTCTTAAAGG | |
| | ACTTTATGGGGAAGTTG | 2174 |
| | CAACTTCCCCATAAAGT | 2175 |
| Haemophilia A | CGTGAAGCTATTCAGCATGAATCAGGAATCTTGGGACCTTTA | 2176 |
| Asp459Asn | CTTTATGGGGAAGTTGGAGACACACTGTTGGTAAGTTGAAGA | |
| aGAC-AAC | AAAGATTTAAGGTCAGGTAAGAAGAAAAAGTCTGGAG | |
| | CTCCAGACTTTTTCTTCTTACCTGACCTTAAATCTTTTCTTCAA | 2177 |
| | CTTACCAACAGTGTGTCTCCAACTTCCCCATAAAGTAAAGGTC | |
| | CCAAGATTCCTGATTCATGCTGAATAGCTTCACG | |
| | AAGTTGGAGACACACTG | 2178 |
| | CAGTGTGTCTCCAACTT | 2179 |
| Haemophilia A | TGTTGATCCTAGTCGTTTTAGGATTTGATCTTAGATCTCGCTTA | 2180 |
| Phe465Cys | TACTTTCAGATTATATTTAAGAATCAAGCAAGCAGACCATATAA | |
| TTT-TGT | CATCTACCCTCACGGAATCACTGATGTCCGTCC | |
| | GGACGGACATCAGTGATTCCGTGAGGGTAGATGTTATATGGT | 2181 |
| | CTGCTTGCTTGATTCTTAAATATAATCTGAAAGTATAAGCGAG | |
| | ATCTAAGATCAAATCCTAAAACGACTAGGATCAACA | |
| | GATTATATTTAAGAATC | 2182 |
| | GATTCTTAAATATAATC | 2183 |
| Haemophilia A | TCGTTTTAGGATTTGATCTTAGATCTCGCTTATACTTTCAGATT | 2184 |
| Ala469Gly | ATATTTAAGAATCAAGCAAGCAGACCATATAACATCTACCCTC | |
| GCA-GGA | ACGGAATCACTGATGTCCGTCCTTTGTATTCAAG | |
| | CTTGAATACAAAGGACGGACATCAGTGATTCCGTGAGGGTAG | 2185 |
| | ATGTTATATGGTCTGCTTGCTTGATTCTTAAATATAATCTGAAA | |
| | GTATAAGCGAGATCTAAGATCAAATCCTAAAACGA | |
| | GAATCAAGCAAGCAGAC | 2186 |
| | GTCTGCTTGCTTGATTC | 2187 |
| Haemophilia A | TTAGGATTTGATCTTAGATCTCGCTTATACTTTCAGATTATATT | 2188 |
| Arg471Gly | TAAGAATCAAGCAAGCAGACCATATAACATCTACCCTCACGG | |
| cAGA-GGA | AATCACTGATGTCCGTCCTTTGTATTCAAGGAGAT | |
| | ATCTCCTTGAATACAAAGGACGGACATCAGTGATTCCGTGAG | 2189 |
| | GGTAGATGTTATATGGTCTGCTTGCTTGATTCTTAAATATAATC | |
| | TGAAAGTATAAGCGAGATCTAAGATCAAATCCTAA | |
| | AAGCAAGCAGACCATAT | 2190 |
| | ATATGGTCTGCTTGCTT | 2191 |
| Haemophilia A | TTGATCTTAGATCTCGCTTATACTTTCAGATTATATTTAAGAAT | 2192 |
| Tyr473Cys | CAAGCAAGCAGACCATATAACATCTACCCTCACGGAATCACT | |
| TAT-TGT | GATGTCCGTCCTTTGTATTCAAGGAGATTACCAAA | |
| | TTTGGTAATCTCCTTGAATACAAAGGACGGACATCAGTGATTC | 2193 |
| | CGTGAGGGTAGATGTTATATGGTCTGCTTGCTTGATTCTTAAA | |
| | TATAATCTGAAAGTATAAGCGAGATCTAAGATCAA | |
| | CAGACCATATAACATCT | 2194 |
| | AGATGTTATATGGTCTG | 2195 |
| Haemophilia A | TTTGATCTTAGATCTCGCTTATACTTTCAGATTATATTTAAGAA | 2196 |
| Tyr473His | TCAAGCAAGCAGACCATATAACATCTACCCTCACGGAATCACT | |
| aTAT-CAT | GATGTCCGTCCTTTGTATTCAAGGAGATTACCAA | |
| | TTGGTAATCTCCTTGAATACAAAGGACGGACATCAGTGATTCC | 2197 |
| | GTGAGGGTAGATGTTATATGGTCTGCTTGCTTGATTCTTAAAT | |
| | ATAATCTGAAAGTATAAGCGAGATCTAAGATCAAA | |
| | GCAGACCATATAACATC | 2198 |
| | GATGTTATATGGTCTGC | 2199 |
| Haemophilia A | TTAGATCTCGCTTATACTTTCAGATTATATTTAAGAATCAAGCA | 2200 |
| Ile475Thr | AGCAGACCATATAACATCTACCCTCACGGAATCACTGATGTCC | |
| ATC-ACC | GTCCTTTGTATTCAAGGAGATTACCAAAAGGTAA | |
| | TTACCTTTTGGTAATCTCCTTGAATACAAAGGACGGACATCAG | 2201 |
| | TGATTCCGTGAGGGTAGATGTTATATGGTCTGCTTGCTTGATT | |
| | CTTAAATATAATCTGAAAGTATAAGCGAGATCTAA | |
| | ATATAACATCTACCCTC | 2202 |
| | GAGGGTAGATGTTATAT | 2203 |
| Haemophilia A | TTATACTTTCAGATTATATTTAAGAATCAAGCAAGCAGACCATA | 2204 |
| Gly479Arg | TAACATCTACCCTCACGGAATCACTGATGTCCGTCCTTTGTAT | |
| cGGA-AGA | TCAAGGAGATTACCAAAAGGTAAATATTCCCTCG | |
| | CGAGGGAATATTTACCTTTTGGTAATCTCCTTGAATACAAAGG | 2205 |
| | ACGGACATCAGTGATTCCGTGAGGGTAGATGTTATATGGTCT | |
| | GCTTGCTTGATTCTTAAATATAATCTGAAAGTATAA | |
| | ACCCTCACGGAATCACT | 2206 |
| | AGTGATTCCGTGAGGGT | 2207 |
| Haemophilia A | CCAATTCTGCCAGGAGAAATATTCAAATATAAATGGACAGTGA | 2208 |
| Thr522Ser | CTGTAGAAGATGGGCCAACTAAATCAGATCCTCGGTGCCTGA | |
| aACT-TCT | CCCGCTATTACTCTAGTTTCGTTAATATGGAGAGAG | |
| | CTCTCTCCATATTAACGAAACTAGAGTAATAGCGGGTCAGGC | 2209 |
| | ACCGAGGATCTGATTTAGTTGGCCCATCTTCTACAGTCACTGT | |
| | CCATTTATATTTGAATATTTCTCCTGGCAGAATTGG | |
| | ATGGGCCAACTAAATCA | 2210 |
| | TGATTTAGTTGGCCCAT | 2211 |
| Haemophilia A | CCAGGAGAAATATTCAAATATAAATGGACAGTGACTGTAGAAG | 2212 |
| Asp525Asn | ATGGGCCAACTAAATCAGATCCTCGGTGCCTGACCCGCTATT | |
| aGAT-AAT | ACTCTAGTTTCGTTAATATGGAGAGAGATCTAGCTT | |
| | AAGCTAGATCTCTCTCCATATTAACGAAACTAGAGTAATAGCG | 2213 |
| | GGTCAGGCACCGAGGATCTGATTTAGTTGGCCCATCTTCTAC | |
| | AGTCACTGTCCATTTATATTTGAATATTTCTCCTGG | |
| | CTAAATCAGATCCTCGG | 2214 |
| | CCGAGGATCTGATTTAG | 2215 |
| Haemophilia A | GAAATATTCAAATATAAATGGACAGTGACTGTAGAAGATGGGC | 2216 |
| Arg527Trp | CAACTAAATCAGATCCTCGGTGCCTGACCCGCTATTACTCTAG | |
| tCGG-TGG | TTTCGTTAATATGGAGAGAGATCTAGCTTCAGGAC | |
| | GTCCTGAAGCTAGATCTCTCTCCATATTAACGAAACTAGAGTA | 2217 |
| | ATAGCGGGTCAGGCACCGAGGATCTGATTTAGTTGGCCCATC | |
| | TTCTACAGTCACTGTCCATTTATATTTGAATATTTC | |
| | CAGATCCTCGGTGCCTG | 2218 |
| | CAGGCACCGAGGATCTG | 2219 |
| Haemophilia A | TATAAATGGACAGTGACTGTAGAAGATGGGCCAACTAAATCA | 2220 |
| Arg531Cys | GATCCTCGGTGCCTGACCCGCTATTACTCTAGTTTCGTTAATA | |
| cCGC-TGC | TGGAGAGAGATCTAGCTTCAGGACTCATTGGCCCTC | |
| | GAGGGCCAATGAGTCCTGAAGCTAGATCTCTCTCCATATTAA | 2221 |
| | CGAAACTAGAGTAATAGCGGGTCAGGCACCGAGGATCTGATT | |
| | TAGTTGGCCCATCTTCTACAGTCACTGTCCATTTATA | |
| | GCCTGACCCGCTATTAC | 2222 |
| | GTAATAGCGGGTCAGGC | 2223 |
| Haemophilia A | TATAAATGGACAGTGACTGTAGAAGATGGGCCAACTAAATCA | 2224 |
| Arg531Gly | GATCCTCGGTGCCTGACCCGCTATTACTCTAGTTTCGTTAATA | |
| cCGC-GGC | TGGAGAGAGATCTAGCTTCAGGACTCATTGGCCCTC | |
| | GAGGGCCAATGAGTCCTGAAGCTAGATCTCTCTCCATATTAA | 2225 |
| | CGAAACTAGAGTAATAGCGGGTCAGGCACCGAGGATCTGATT | |
| | TAGTTGGCCCATCTTCTACAGTCACTGTCCATTTATA | |
| | GCCTGACCCGCTATTAC | 2226 |
| | GTAATAGCGGGTCAGGC | 2227 |
| Haemophilia A | ATAAATGGACAGTGACTGTAGAAGATGGGCCAACTAAATCAG | 2228 |
| Arg531His | ATCCTCGGTGCCTGACCCGlCTATTACTCTAGTTTCGTTAATAT | |
| CGC-CAC | GGAGAGAGATCTAGCTTCAGGACTCATTGGCCCTCT | |
| | AGAGGGCCAATGAGTCCTGAAGCTAGATCTCTCTCCATATTA | 2229 |
| | ACGAAACTAGAGTAATAGCGGGTCAGGCACCGAGGATCTGAT | |
| | TTAGTTGGCCCATCTTCTACAGTCACTGTCCATTTAT | |
| | CCTGACCCGCTATTACT | 2230 |
| | AGTAATAGCGGGTCAGG | 2231 |
| Haemophilia A | ACAGTGACTGTAGAAGATGGGCCAACTAAATCAGATCCTCGG | 2232 |
| Ser534Pro | TGCCTGACCCGCTATTACTCTAGTTTCGTTAATATGGAGAGAG | |
| cTCT-CCT | ATCTAGCTTCAGGACTCATTGGCCCTCTCCTCATCT | |
| | AGATGAGGAGAGGGCCAATGAGTCCTGAAGCTAGATCTCTCT | 2233 |
| | CCATATTAACGAAACTAGAGTAATAGCGGGTCAGGCACCGAG | |
| | GATCTGATTTAGTTGGCCCATCTTCTACAGTCACTGT | |
| | GCTATTACTCTAGTTTC | 2234 |
| | GAAACTAGAGTAATAGC | 2235 |
| Haemophilia A | GTGACTGTAGAAGATGGGCCAACTAAATCAGATCCTCGGTGC | 2236 |
| Ser535Gly | CTGACCCGCTATTACTCTAGTTTCGTTAATATGGAGAGAGATC | |
| tAGT-GGT | TAGCTTCAGGACTCATTGGCCCTCTCCTCATCTGCT | |
| | AGCAGATGAGGAGAGGGCCAATGAGTCCTGAAGCTAGATCT | 2237 |
| | CTCTCCATATTAACGAAACTAGAGTAATAGCGGGTCAGGCAC | |
| | CGAGGATCTGATTTAGTTGGCCCATCTTCTACAGTCAC | |
| | ATTACTCTAGTTTCGTT | 2238 |
| | AACGAAACTAGAGTAAT | 2239 |
| Haemophilia A | TAGAAGATGGGCCAACTAAATCAGATCCTCGGTGCCTGACCC | 2240 |
| Val537Asp | GCTATTACTCTAGTTTCGTTAATATGGAGAGAGATCTAGCTTC | |
| GTT-GAT | AGGACTCATTGGCCCTCTCCTCATCTGCTACAAAGA | |
| | TCTTTGTAGCAGATGAGGAGAGGGCCAATGAGTCCTGAAGCT | 2241 |
| | AGATCTCTCTCCATATTAACGAAACTAGAGTAATAGCGGGTCA | |
| | GGCACCGAGGATCTGATTTAGTTGGCCCATCTTCTA | |
| | TAGTTTCGTTAATATGG | 2242 |
| | CCATATTAACGAAACTA | 2243 |
| Haemophilia A | CAACTAAATCAGATCCTCGGTGCCTGACCCGCTATTACTCTAG | 2244 |
| Arg541Thr | TTTCGTTAATATGGAGAGAGATCTAGCTTCAGGACTCATTGGC | |
| AGA-ACA | CCTCTCCTCATCTGCTACAAAGAATCTGTAGATCA | |
| | TGATCTACAGATTCTTTGTAGCAGATGAGGAGAGGGCCAATG | 2245 |
| | AGTCCTGAAGCTAGATCTCTCTCCATATTAACGAAACTAGAGT | |
| | AATAGCGGGTCAGGCACCGAGGATCTGATTTAGTTG | |
| | TATGGAGAGAGATCTAG | 2246 |
| | CTAGATCTCTCTCCATA | 2247 |
| Haemophilia A | CTAAATCAGATCCTCGGTGCCTGACCCGCTATTACTCTAGTTT | 2248 |
| Asp542Gly | CGTTAATATGGAGAGAGATCTAGCTTCAGGACTCATTGGCCC | |
| GAT-GGT | TCTCCTCATCTGCTACAAAGAATCTGTAGATCAAAG | |
| | CTTTGATCTACAGATTCTTTGTAGCAGATGAGGAGAGGGCCA | 2249 |
| | ATGAGTCCTGAAGCTAGATCTCTCTCCATATTAACGAAACTAG | |
| | AGTAATAGCGGGTCAGGCACCGAGGATCTGATTTAG | |
| | GGAGAGAGATCTAGCTT | 2250 |
| | AAGCTAGATCTCTCTCC | 2251 |
| Haemophilia A | ACTAAATCAGATCCTCGGTGCCTGACCCGCTATTACTCTAGTT | 2252 |
| Asp542His | TCGTTAATATGGAGAGAGATCTAGCTTCAGGACTCATTGGCC | |
| aGAT-CAT | CTCTCCTCATCTGCTACAAAGAATCTGTAGATCAAA | |
| | TTTGATCTACAGATTCTTTGTAGCAGATGAGGAGAGGGCCAA | 2253 |
| | TGAGTCCTGAAGCTAGATCTCTCTCCATATTAACGAAACTAGA | |
| | GTAATAGCGGGTCAGGCACCGAGGATCTGATTTAGT | |
| | TGGAGAGAGATCTAGCT | 2254 |
| | AGCTAGATCTCTCTCCA | 2255 |
| Haemophilia A | ACTAAATCAGATCCTCGGTGCCTGACCCGCTATTACTCTAGTT | 2256 |
| Asp542Tyr | TCGTTAATATGGAGAGAGATCTAGCTTCAGGACTCATTGGCC | |
| aGAT-TAT | CTCTCCTCATCTGCTACAAAGAATCTGTAGATCAAA | |
| | TTTGATCTACAGATTCTTTGTAGCAGATGAGGAGAGGGCCAA | 2257 |
| | TGAGTCCTGAAGCTAGATCTCTCTCCATATTAACGAAACTAGA | |
| | GTAATAGCGGGTCAGGCACCGAGGATCTGATTTAGT | |
| | TGGAGAGAGATCTAGCT | 2258 |
| | AGCTAGATCTCTCTCCA | 2259 |
| Haemophilia A | GTTAATATGGAGAGAGATCTAGCTTCAGGACTCATTGGCCCT | 2260 |
| Glu551Term | CTCCTCATCTGCTACAAAGAATCTGTAGATCAAAGAGGAAACC | |
| aGAA-TAA | AGGTGAGTTCTTGCCTTTCCAAGTGCTGGGTTTCAT | |
| | ATGAAACCCAGCACTTGGAAAGGCAAGAACTCACCTGGTTTC | 2261 |
| | CTCTTTGATCTACAGATTCTTTGTAGCAGATGAGGAGAGGGC | |
| | CAATGAGTCCTGAAGCTAGATCTCTCTCCATATTAAC | |
| | GCTACAAAGAATCTGTA | 2262 |
| | TACAGATTCTTTGTAGC | 2263 |
| Haemophilia A | ATATGGAGAGAGATCTAGCTTCAGGACTCATTGGCCCTCTCC | 2264 |
| Ser558Phe | TCATCTGCTACAAAGAATCTGTAGATCAAAGAGGAAACCAGG | |
| TCT-TTT | TGAGTTCTTGCCTTTCCAAGTGCTGGGTTTCATTCTC | |
| | GAGAATGAAACCCAGCACTTGGAAAGGCAAGAACTCACCTGG | 2265 |
| | TTTCCTCTTTGATCTACAGATTCTTTGTAGCAGATGAGGAGAG | |
| | GGCCAATGAGTCCTGAAGCTAGATCTCTCTCCATAT | |
| | CAAAGAATCTGTAGATC | 2266 |
| | GATCTACAGATTCTTTG | 2267 |
| Haemophilia A | TGGAGAGAGATCTAGCTTCAGGACTCATTGGCCCTCTCCTCA | 2268 |
| Val559Ala | TCTGCTACAAAGAATCTGTAGATCAAAGAGGAAACCAGGTGA | |
| GTA-GCA | GTTCTTGCCTTTCCAAGTGCTGGGTTTCATTCTCAGT | |
| | ACTGAGAATGAAACCCAGCACTTGGAAAGGCAAGAACTCACC | 2269 |
| | TGGTTTCCTCTTTGATCTACAGATTCTTTGTAGCAGATGAGGA | |
| | GAGGGCCAATGAGTCCTGAAGCTAGATCTCTCTCCA | |
| | AGAATCTGTAGATCAAA | 2270 |
| | TTTGATCTACAGATTCT | 2271 |

### EXAMPLE 15

### Hemophilia—Factor IX Deficiency

The attached table discloses the correcting oligonucleotide base sequences for the Factor IX oligonucleotides of the invention.

TABLE 17
Factor IX Mutations And Genome-Correcting Oligos

| Clinical Phenotype & | | SEQ ID |
|---|---|---|
| Mutation | Correcting Oligos | NO: |
| Haemophilia B | ATTTCAGTTTTTCTTGATCATGAAAACGCCAACAAAATTCTGAA | 2272 |
| Asn2Asp | TCGGCCAAAGAGGTATAATTCAGGTAAATTGGAAGAGTTTGTT | |
| tAAT-GAT | CAAGGGAACCTTGAGAGAGAATGTATGGAAGAAA | |
| | TTTCTTCCATACATTCTCTCTCAAGGTTCCCTTGAACAAACTCT | 2273 |
| | TCCAATTTACCTGAATTATACCTCTTTGGCCGATTCAGAATTTT | |
| | GTTGGCGTTTTCATGATCAAGAAAAACTGAAAT | |
| | AGAGGTATAATTCAGGT | 2274 |
| | ACCTGAATTATACCTCT | 2275 |
| Haemophilia B | TTTCAGTTTTTCTTGATCATGAAAACGCCAACAAAATTCTGAAT | 2276 |
| Asn2Ile | CGGCCAAAGAGGTATAATTCAGGTAAATTGGAAGAGTTTGTT | |
| AAT-ATT | CAAGGGAACCTTGAGAGAGAATGTATGGAAGAAAA | |
| | TTTTCTTCCATACATTCTCTCTCAAGGTTCCCTTGAACAAACTC | 2277 |
| | TTCCAATTTACCTGAATTATACCTCTTTGGCCGATTCAGAATTT | |
| | TGTTGGCGTTTTCATGATCAAGAAAAACTGAAA | |
| | GAGGTATAATTCAGGTA | 2278 |
| | TACCTGAATTATACCTC | 2279 |
| Haemophilia B | ATTTCAGTTTTTCTTGATCATGAAAACGCCAACAAAATTCTGAA | 2280 |
| Asn2Tyr | TCGGCCAAAGAGGTATAATTCAGGTAAATTGGAAGAGTTTGTT | |
| tAAT-TAT | CAAGGGAACCTTGAGAGAGAATGTATGGAAGAAA | |
| | TTTCTTCCATACATTCTCTCTCAAGGTTCCCTTGAACAAACTCT | 2281 |
| | TCCAATTTACCTGAATTATACCTCTTTGGCCGATTCAGAATTTT | |
| | GTTGGCGTTTTCATGATCAAGAAAAACTGAAAT | |
| | AGAGGTATAATTCAGGT | 2282 |
| | ACCTGAATTATACCTCT | 2283 |
| Haemophilia B | TCAGTTTTTCTTGATCATGAAAACGCCAACAAAATTCTGAATC | 2284 |
| Ser3Pro | GGCCAAAGAGGTATAATTCAGGTAAATTGGAAGAGTTTGTTC | |
| tTCA-CCA | AAGGGAACCTTGAGAGAGAATGTATGGAAGAAAAGT | |
| | ACTTTTCTTCCATACATTCTCTCTCAAGGTTCCCTTGAACAAAC | 2285 |
| | TCTTCCAATTTACCTGAATTATACCTCTTTGGCCGATTCAGAAT | |
| | TTTGTTGGCGTTTTCATGATCAAGAAAAACTGA | |
| | GGTATAATTCAGGTAAA | 2286 |
| | TTTACCTGAATTATACC | 2287 |
| Haemophilia B | TTTTTCTTGATCATGAAAACGCCAACAAAATTCTGAATCGGCC | 2288 |
| Gly4Asp | AAAGAGGTATAATTCAGGTAAATTGGAAGAGTTTGTTCAAGG | |
| GGT-GAT | GAACCTTGAGAGAGAATGTATGGAAGAAAAGTGTAG | |
| | CTACACTTTTCTTCCATACATTCTCTCTCAAGGTTCCCTTGAAC | 2289 |
| | AAACTCTTCCAATTTACCTGAATTATACCTCTTTGGCCGATTCA | |
| | GAATTTTGTTGGCGTTTTCATGATCAAGAAAAA | |
| | TAATTCAGGTAAATTGG | 2290 |
| | CCAATTTACCTGAATTA | 2291 |
| Haemophilia B | GTTTTTCTTGATCATGAAAACGCCAACAAAATTCTGAATCGGC | 2292 |
| Gly4Ser | CAAAGAGGTATAATTCAGGTAAATTGGAAGAGTTTGTTCAAGG | |
| aGGT-AGT | GAACCTTGAGAGAGAATGTATGGAAGAAAAGTGTA | |
| | TACACTTTTCTTCCATACATTCTCTCTCAAGGTTCCCTTGAACA | 2293 |
| | AACTCTTCCAATTTACCTGAATTATACCTCTTTGGCCGATTCA | |
| | GAATTTTGTTGGCGTTTTCATGATCAAGAAAAAC | |
| | ATAATTCAGGTAAATTG | 2294 |
| | CAATTTACCTGAATTAT | 2295 |
| Haemophilia B | TTTCTTGATCATGAAAACGCCAACAAAATTCTGAATCGGCCAA | 2296 |
| LysSGlu | AGAGGTATAATTCAGGTAAATTGGAAGAGTTTGTTCAAGGGA | |
| tAAA-GAA | ACCTTGAGAGAGAATGTATGGAAGAAAAGTGTAGTT | |
| | AACTACACTTTTCTTCCATACATTCTCTCTCAAGGTTCCCTTGA | 2297 |
| | ACAAACTCTTCCAATTTACCTGAATTATACCTCTTTGGCCGATT | |
| | CAGAATTTTGTTGGCGTTTTCATGATCAAGAAA | |
| | ATTCAGGTAAATTGGAA | 2298 |
| | TTCCAATTTACCTGAAT | 2299 |
| Haemophilia B | ATCATGAAAACGCCAACAAAATTCTGAATCGGCCAAAGAGGT | 2300 |
| Glu7Ala | ATAATTCAGGTAAATTGGAAGAGTTTGTTCAAGGGAACCTTGA | |
| GAA-GCA | GAGAGAATGTATGGAAGAAAAGTGTAGTTTTGAAGA | |
| | TCTTCAAAACTACACTTTTCTTCCATACATTCTCTCTCAAGGTT | 2301 |
| | CCCTTGAACAAACTCTTCCAATTTACCTGAATTATACCTCTTTG | |
| | GCCGATTCAGAATTTTGTTGGCGTTTTCATGAT | |
| | TAAATTGGAAGAGTTTG | 2302 |
| | CAAACTCTTCCMTTTA | 2303 |
| Haemophilia B | GATCATGAAAACGCCAACAAAATTCTGAATCGGCCAAAGAGG | 2304 |
| Glu7Lys | TATAATTCAGGTAAATTGGAAGAGTTTGTTCAAGGGAACCTTG | |
| gGAA-AAA | AGAGAGAATGTATGGAAGAAAAGTGTAGTTTTGAAG | |
| | CTTCAAAACTACACTTTTCTTCCATACATTCTCTCTCAAGGTTC | 2305 |
| | CCTTGAACAAACTCTTCCAATTTACCTGAATTATACCTCTTTGG | |
| | CCGATTCAGAATTTTGTTGGCGTTTTCATGATC | |
| | GTAAATTGGAAGAGTTT | 2306 |
| | AAACTCTTCCAATTTAC | 2307 |
| Haemophilia B | ATCATGAAAACGCCAACAAAATTCTGAATCGGCCAAAGAGGT | 2308 |
| Glu7Val | ATAATTCAGGTAAATTGGAAGAGTTTGTTCAAGGGAACCTTGA | |
| GAA-GTA | GAGAGAATGTATGGAAGAAAAGTGTAGTTTTGAAGA | |
| | TCTTCAAAACTACACTTTTCTTCCATACATTCTCTCTCAAGGTT | 2309 |
| | CCCTTGAACAAACTCTTCCAATTTACCTGAATTATACCTCTTTG | |
| | GCCGATTCAGAATTTTGTTGGCGTTTTCATGAT | |
| | TAAATTGGAAGAGTTTG | 2310 |
| | CAAACTCTTCCAATTTA | 2311 |
| Haemophilia B | ATGAAAACGCCAACAAAATTCTGAATCGGCCAAAGAGGTATA | 2312 |
| Glu8Ala | ATTCAGGTAAATTGGAAGAGTTTGTTCAAGGGAACCTTGAGA | |
| GAG-GCG | GAGAATGTATGGAAGAAAAGTGTAGTTTTGAAGAAGC | |
| | GCTTCTTCAAAACTACACTTTTCTTCCATACATTCTCTCTCAAG | 2313 |
| | GTTCCCTTGAACAAACTCTTCCAATTTACCTGAATTATACCTCT | |
| | TTGGCCGATTCAGAATTTTGTTGGCGTTTTCAT | |
| | ATTGGAAGAGTTTGTTC | 2314 |
| | GAACAAACTCTTCCAAT | 2315 |
| Haemophilia B | ATGAAAACGCCAACAAAATTCTGAATCGGCCAAAGAGGTATA | 2316 |
| Glu8Gly | ATTCAGGTAAATTGGAAGAGTTTGTTCAAGGGAACCTTGAGA | |
| GAG-GGG | GAGAATGTATGGAAGAAAAGTGTAGTTTTGAAGAAGC | |
| | GCTTCTTCAAAACTACACTTTTCTTCCATACATTCTCTCTCAAG | 2317 |
| | GTTCCCTTGAACAAACTCTTCCAATTTACCTGAATTATACCTCT | |
| | TTGGCCGATTCAGAATTTTGTTGGCGTTTTCAT | |
| | ATTGGAAGAGTTTGTTC | 2318 |
| | GAACAAACTCTTCCAAT | 2319 |
| Haemophilia B | AAAACGCCAACAAAATTCTGAATCGGCCAAAGAGGTATAATTC | 2320 |
| Phe9Cys | AGGTAAATTGGAAGAGTTTGTTCAAGGGAACCTTGAGAGAGA | |
| TTT-TGT | ATGTATGGAAGAAAAGTGTAGTTTTGAAGAAGCACG | |
| | CGTGCTTCTTCAAAACTACACTTTTCTTCCATACATTCTCTCTC | 2321 |
| | AAGGTTCCCTTGAACAAACTCTTCCAATTTACCTGAATTATAC | |
| | CTCTTTGGCCGATTCAGAATTTTGTTGGCGTTTT | |
| | GGAAGAGTTTGTTCAAG | 2322 |
| | CTTGAACAAACTCTTCC | 2323 |
| Haemophilia B | GAAAACGCCAACAAAATTCTGAATCGGCCAAAGAGGTATAAT | 2324 |
| Phe9Ile | TCAGGTAAATTGGAAGAGTTTGTTCAAGGGAACCTTGAGAGA | |
| gTTT-ATT | GAATGTATGGAAGAAAAGTGTAGTTTTGAAGAAGCAC | |
| gTTT-ATT | GTGCTTCTTCAAAACTACACTTTTCTTCCATACATTCTCTCTCA | 2325 |
| | AGGTTCCCTTGAACAAACTCTTCCAATTTACCTGAATTATACC | |
| | TCTTTGGCCGATTCAGAATTTTGTTGGCGTTTTC | |
| | TGGAAGAGTTTGTTCAA | 2326 |
| | TTGAACAAACTCTTCCA | 2327 |
| Haemophilia B | TTACATTTCAGTTTTTCTTGATCATGAAAACGCCAACAAAATTC | 2328 |
| Arg(−1)Ser | TGAATCGGCCAAAGAGGTATAATTCAGGTAAATTGGAAGAGT | |
| AGGt-AGC | TTGTTCAAGGGAACCTTGAGAGAGAATGTATGGAA | |
| | TTCCATACATTCTCTCTCAAGGTTCCCTTGAACAAACTCTTCC | 2329 |
| | AATTTACCTGAATTATACCTCTTTGGCCGATTCAGAATTTTGTT | |
| | GGCGTTTTCATGATCAAGAAAAACTGAAATGTAA | |
| | CCAAAGAGGTATAATTC | 2330 |
| | GAATTATACCTCTTTGG | 2331 |
| Haemophilia B | TTTACATTTCAGTTTTTCTTGATCATGAAAACGCCAACAAAATT | 2332 |
| Arg(−1)Thr | CTGAATCGGCCAAAGAGGTATAATTCAGGTAAATTGGAAGAG | |
| AGG-ACG | TTTGTTCAAGGGAACCTTGAGAGAGAATGTATGGA | |
| | TCCATACATTCTCTCTCAAGGTTCCCTTGAACAAACTCTTCCA | 2333 |
| | ATTTACCTGAATTATACCTCTTTGGCCGATTCAGAATTTTGTTG | |
| | GCGTTTTCATGATCAAGAAAAACTGAAATGTAAA | |
| | GCCAAAGAGGTATAATT | 2334 |
| | AATTATACCTCTTTGGC | 2335 |
| Haemophilia B | CTTTTACATTTCAGTTTTTCTTGATCATGAAAACGCCAACAAAA | 2336 |
| Lys(−2)Asn | TTCTGAATCGGCCAAAGAGGTATAATTCAGGTAAATTGGAAG | |
| AAGa-AAT | AGTTTGTTCAAGGGAACCTTGAGAGAGAATGTATG | |
| | CATACATTCTCTCTCAAGGTTCCCTTGAACAAACTCTTCCAATT | 2337 |
| | TACCTGAATTATACCTCTTTGGCCGATTCAGAATTTTGTTGGC | |
| | GTTTTCATGATCAAGAAAAACTGAAATGTAAAAG | |
| | CGGCCAAAGAGGTATAA | 2338 |
| | TTATACCTCTTTGGCCG | 2339 |
| Haemophilia B | AATTATTCTTTTACATTTCAGTTTTTCTTGATCATGAAAACGCC | 2340 |
| Arg(−4)Gln | AACAAAATTCTGAATCGGCCAAAGAGGTATAATTCAGGTAAAT | |
| CGG-CAG | TGGAAGAGTTTGTTCAAGGGAACCTTGAGAGAGA | |
| | TCTCTCTCAAGGTTCCCTTGAACAAACTCTTCCAATTTACCTG | 2341 |
| | AATTATACCTCTTTGGCCGATTCAGAATTTTGTTGGCGTTTTC | |
| | ATGATCAAGAAAAACTGAAATGTAAAAGAATAATT | |
| | TCTGAATCGGCCAAAGA | 2342 |
| | TCTTTGGCCGATTCAGA | 2343 |
| Haemophilia B | AATTATTCTTTTACATTTCAGTTTTTCTTGATCATGAAAACGCC | 2344 |
| Arg(−4)Leu | AACAAAATTCTGAATCGGCCAAAGAGGTATAATTCAGGTAAAT | |
| CGG-CTG | TGGAAGAGTTTGTTCAAGGGAACCTTGAGAGAGA | |
| | TCTCTCTCAAGGTTCCCTTGAACAAACTCTTCCAATTTACCTG | 2345 |
| | AATTATACCTCTTTGGCCGATTCAGAATTTTGTTGGCGTTTTC | |
| | ATGATCAAGAAAAACTGAAATGTAAAAGAATAATT | |
| | TCTGAATCGGCCAAAGA | 2346 |
| | TCTTTGGCCGATTCAGA | 2347 |
| Haemophilia B | GAATTATTCTTTTACATTTCAGTTTTTCTTGATCATGAAAACGC | 2348 |
| Arg(−4)Trp | CAACAAAATTCTGAATCGGCCAAAGAGGTATAATTCAGGTAAA | |
| tCGG-TGG | TTGGAAGAGTTTGTTCAAGGGAACCTTGAGAGAG | |
| | CTCTCTCAAGGTTCCCTTGAACAAACTCTTCCAATTTACCTGA | 2349 |
| | ATTATACCTCTTTGGCCGATTCAGAATTTTGTTGGCGTTTTCAT | |
| | GATCAAGAAAAACTGAAATGTAAAAGAATAATTC | |
| | TTCTGAATCGGCCAAAG | 2350 |
| | CTTTGGCCGATTCAGAA | 2351 |
| Haemophilia B | GCCAACAAAATTCTGAATCGGCCAAAGAGGTATAATTCAGGT | 2352 |
| GinllTerm | AAATTGGAAGAGTTTGTTCAAGGGAACCTTGAGAGAGAATGT | |
| tCAA-TAA | ATGGAAGAAAAGTGTAGTTTTGAAGAAGCACGAGAAG | |
| | CTTCTCGTGCTTCTTCAAAACTACACTTTTCTTCCATACATTCT | 2353 |
| | CTCTCAAGGTTCCCTTGAACAAACTCTTCCAATTTACCTGAAT | |
| | TATACCTCTTTGGCCGATTCAGAATTTTGTTGGC | |
| | AGTTTGTTCAAGGGAAC | 2354 |
| | GTTCCCTTGAACAAACT | 2355 |
| Haemophilia B | ACAAAATTCTGAATCGGCCAAAGAGGTATAATTCAGGTAAATT | 2356 |
| Gly12Ala | GGAAGAGTTTGTTCAAGGGAACCTTGAGAGAGAATGTATGGA | |
| GGG-GCG | AGAAAAGTGTAGTTTTGAAGAAGCACGAGAAGTTTT | |
| | AAAACTTCTCGTGCTTCTTCAAAACTACACTTTTCTTCCATACA | 2357 |
| | TTCTCTCTCAAGGTTCCCTTGAACAAACTCTTCCAATTTACCT | |
| | GAATTATACCTCTTTGGCCGATTCAGAATTTTGT | |
| | TGTTCAAGGGAACCTTG | 2358 |
| | CAAGGTTCCCTTGAACA | 2359 |
| Haemophilia B | AACAAAATTCTGAATCGGCCAAAGAGGTATAATTCAGGTAAAT | 2360 |
| Gly12Arg | TGGAAGAGTTTGTTCAAGGGAACCTTGAGAGAGAATGTATGG | |
| aGGG-AGG | AAGAAAAGTGTAGTTTTGAAGAAGCACGAGAAGTTT | |
| | AAACTTCTCGTGCTTCTTCAAAACTACACTTTTCTTCCATACAT | 2361 |
| | TCTCTCTCAAGGTTCCCTTGAACAAACTCTTCCAATTTACCTG | |
| | AATTATACCTCTTTGGCCGATTCAGAATTTTGTT | |
| | TTGTTCAAGGGAACCTT | 2362 |
| | AAGGTTCCCTTGAACAA | 2363 |
| Haemophilia B | ACAAAATTCTGAATCGGCCAAAGAGGTATAATTCAGGTAAATT | 2364 |
| Gly12Glu | GGAAGAGTTTGTTCAAGGGAACCTTGAGAGAGAATGTATGGA | |
| GGG-GAG | AGAAAAGTGTAGTTTTGAAGAAGCACGAGAAGTTTT | |
| | AAAACTTCTCGTGCTTCTTCAAAACTACACTTTTCTTCCATACA | 2365 |
| | TTCTCTCTCAAGGTTCCCTTGAACAAACTCTTCCAATTTACCT | |
| | GAATTATACCTCTTTGGCCGATTCAGAATTTTGT | |
| | TGTTCAAGGGAACCTTG | 2366 |
| | CAAGGTTCCCTTGAACA | 2367 |
| Haemophilia B | CGGCCAAAGAGGTATAATTCAGGTAAATTGGAAGAGTTTGTT | 2368 |
| Glu17Gln | CAAGGGAACCTTGAGAGAGAATGTATGGAAGAAAAGTGTAGT | |
| aGAA-CAA | TTTGAAGAAGCACGAGAAGTTTTTGAAAACACTGAAA | |
| | TTTCAGTGTTTTCAAAAACTTCTCGTGCTTCTTCAAAACTACAC | 2369 |
| | TTTTCTTCCATACATTCTCTCTCAAGGTTCCCTTGAACAAACTC | |
| | TTCCAATTTACCTGAATTATACCTCTTTGGCCG | |
| | TTGAGAGAGAATGTATG | 2370 |
| | CATACATTCTCTCTCAA | 2371 |
| Haemophilia B | CGGCCAAAGAGGTATAATTCAGGTAAATTGGAAGAGTTTGTT | 2372 |
| Glu17Lys | CAAGGGAACCTTGAGAGAGAATGTATGGAAGAAAAGTGTAGT | |
| aGAA-AAA | TTTGAAGAAGCACGAGAAGTTTTTGAAAACACTGAAA | |
| | TTTCAGTGTTTTCAAAAACTTCTCGTGCTTCTTCAAAACTACAC | 2373 |
| | TTTTCTTCCATACATTCTCTCTCAAGGTTCCCTTGAACAAACTC | |
| | TTCCAATTTACCTGAATTATACCTCTTTGGCCG | |
| | TTGAGAGAGAATGTATG | 2374 |
| | CATACATTCTCTCTCAA | 2375 |
| Haemophilia B | CCAAAGAGGTATAATTCAGGTAAATTGGAAGAGTTTGTTCAAG | 2376 |
| Cys18Arg | GGAACCTTGAGAGAGAATGTATGGAAGAAAAGTGTAGTTTTG | |
| aTGT-CGT | AAGAAGCACGAGAAGTTTTTGAAAACACTGAAAGAA | |
| | TTCTTTCAGTGTTTTCAAAAACTTCTCGTGCTTCTTCAAAACTA | 2377 |
| | CACTTTTCTTCCATACATTCTCTCTCAAGGTTCCCTTGAACAAA | |
| | CTCTTCCAATTTACCTGAATTATACCTCTTTGG | |
| | AGAGAGAATGTATGGAA | 2378 |
| | TTCCATACATTCTCTCT | 2379 |
| Haemophilia B | CAAAGAGGTATAATTCAGGTAAATTGGAAGAGTTTGTTCAAGG | 2380 |
| Cys18Tyr | GAACCTTGAGAGAGAATGTATGGAAGAAAAGTGTAGTTTTGA | |
| TGT-TAT | AGAAGCACGAGAAGTTTTTGAAAACACTGAAAGAAC | |
| | GTTCTTTCAGTGTTTTCAAAAACTTCTCGTGCTTCTTCAAAACT | 2381 |
| | ACACTTTTCTTCCATACATTCTCTCTCAAGGTTCCCTTGAACAA | |
| | ACTCTTCCAATTTACCTGAATTATACCTCTTTG | |
| | GAGAGAATGTATGGAAG | 2382 |
| | CTTCCATACATTCTCTC | 2383 |
| Haemophilia B | GGTATAATTCAGGTAAATTGGAAGAGTTTGTTCAAGGGAACCT | 2384 |
| Glu20Val | TGAGAGAGAATGTATGGAAGAAAAGTGTAGTTTTGAAGAAGC | |
| GAA-GTA | ACGAGAAGTTTTTGAAAACACTGAAAGAACAGTGAG | |
| | CTCACTGTTCTTTCAGTGTTTTCAAAAACTTCTCGTGCTTCTTC | 2385 |
| | AAAACTACACTTTTCTTCCATACATTCTCTCTCAAGGTTCCCTT | |
| | GAACAAACTCTTCCAATTTACCTGAATTATACC | |
| | ATGTATGGAAGAAAAGT | 2386 |
| | ACTTTTCTTCCATACAT | 2387 |
| Haemophilia B | TATAATTCAGGTAAATTGGAAGAGTTTGTTCAAGGGAACCTTG | 2388 |
| Glu21Lys | AGAGAGAATGTATGGAAGAAAAGTGTAGTTTTGAAGAAGCAC | |
| aGAA-AAA | GAGAAGTTTTTGAAAACACTGAAAGAACAGTGAGTA | |
| | TACTCACTGTTCTTTCAGTGTTTTCAAAAACTTCTCGTGCTTCT | 2389 |
| | TCAAAACTACACTTTTCTTCCATACATTCTCTCTCAAGGTTCCC | |
| | TTGAACAAACTCTTCCAATTTACCTGAATTATA | |
| | GTATGGAAGAAAAGTGT | 2390 |
| | ACACTTTTCTTCCATAC | 2391 |
| Haemophilia B | TCAGGTAAATTGGAAGAGTTTGTTCAAGGGAACCTTGAGAGA | 2392 |
| Cys23Arg | GAATGTATGGAAGAAAAGTGTAGTTTTGAAGAAGCACGAGAA | |
| gTGT-CGT | GTTTTTGAAAACACTGAAAGAACAGTGAGTATTTCCA | |
| | TGGAAATACTCACTGTTCTTTCAGTGTTTTCAAAAACTTCTCGT | 2393 |
| | GCTTCTTCAAAACTACACTTTTCTTCCATACATTCTCTCTCAAG | |
| | GTTCCCTTGAACAAACTCTTCCAATTTACCTGA | |
| | AAGAAAAGTGTAGTTTT | 2394 |
| | AAAACTACACTTTTCTT | 2395 |
| Haemophilia B | CAGGTAAATTGGAAGAGTTTGTTCAAGGGAACCTTGAGAGAG | 2396 |
| Cys23Tyr | AATGTATGGAAGAAAAGTGTAGTTTTGAAGAAGCACGAGAAG | |
| TGT-TAT | TTTTTGAAAACACTGAAAGAACAGTGAGTATTTCCAC | |
| | GTGGAAATACTCACTGTTCTTTCAGTGTTTTCAAAAACTTCTC | 2397 |
| | GTGCTTCTTCAAAACTACACTTTTCTTCCATACATTCTCTCTCA | |
| | AGGTTCCCTTGAACAAACTCTTCCAATTTACCTG | |
| | AGAAAAGTGTAGTTTTG | 2398 |
| | CAAAACTACACTTTTCT | 2399 |
| Haemophilia B | AATTGGAAGAGTTTGTTCAAGGGAACCTTGAGAGAGAATGTA | 2400 |
| Phe25Ser | TGGAAGAAAAGTGTAGTTTTGAAGAAGCACGAGAAGTTTTTG | |
| TTT-TCT | AAAACACTGAAAGAACAGTGAGTATTTCCACATAATA | |
| | TATTATGTGGAAATACTCACTGTTCTTTCAGTGTTTTCAAAAAC | 2401 |
| | TTCTCGTGCTTCTTCAAAACTACACTTTTCTTCCATACATTCTC | |
| | TCTCAAGGTTCCCTTGAACAAACTCTTCCAATT | |
| | GTGTAGTTTTGAAGAAG | 2402 |
| | CTTCTTCAAAACTACAC | 2403 |
| Haemophilia B | TTGGAAGAGTTTGTTCAAGGGAACCTTGAGAGAGAATGTATG | 2404 |
| Glu26Gln | GAAGAAAAGTGTAGTTTTGAAGAAGCACGAGAAGTTTTTGAAA | |
| tGAA-CAA | ACACTGAAAGAACAGTGAGTATTTCCACATAATACC | |
| | GGTATTATGTGGAAATACTCACTGTTCTTTCAGTGTTTTCAAAA | 2405 |
| | ACTTCTCGTGCTTCTTCAAAACTACACTTTTCTTCCATACATTC | |
| | TCTCTCAAGGTTCCCTTGAACAAACTCTTCCAA | |
| | GTAGTTTTGAAGAAGCA | 2406 |
| | TGCTTCTTCAAAACTAC | 2407 |
| Haemophilia B | AAGAGTTTGTTCAAGGGAACCTTGAGAGAGAATGTATGGAAG | 2408 |
| Glu27Ala | AAAAGTGTAGTTTTGAAGAAGCACGAGAAGTTTTTGAAAACAC | |
| GAA-GCA | TGAAAGAACAGTGAGTATTTCCACATAATACCCTTC | |
| | GAAGGGTATTATGTGGAAATACTCACTGTTCTTTCAGTGTTTT | 2409 |
| | CAAAAACTTCTCGTGCTTCTTCAAAACTACACTTTTCTTCCATA | |
| | CATTCTCTCTCAAGGTTCCCTTGAACAAACTCTT | |
| | TTTTGAAGAAGCACGAG | 2410 |
| | CTCGTGCTTCTTCAAAA | 2411 |
| Haemophilia B | AGAGTTTGTTCAAGGGAACCTTGAGAGAGAATGTATGGAAGA | 2412 |
| Glu27Asp | AAAGTGTAGTTTTGAAGAAGCACGAGAAGTTTTTGAAAACACT | |
| GAAg-GAC | GAAAGAACAGTGAGTATTTCCACATAATACCCTTCA | |
| | TGAAGGGTATTATGTGGAAATACTCACTGTTCTTTCAGTGTTT | 2413 |
| | TCAAAAACTTCTCGTGCTTCTTCAAAACTACACTTTTCTTCCAT | |
| | ACATTCTCTCTCAAGGTTCCCTTGAACAAACTCT | |
| | TTTGAAGAAGCACGAGA | 2414 |
| | TCTCGTGCTTCTTCAAA | 2415 |
| Haemophilia B | GAAGAGTTTGTTCAAGGGAACCTTGAGAGAGAATGTATGGAA | 2416 |
| Glu27Lys | GAAAAGTGTAGTTTTGAAGAAGCACGAGAAGTTTTTGAAAACA | |
| aGAA-AAA | CTGAAAGAACAGTGAGTATTTCCACATAATACCCTT | |
| | AAGGGTATTATGTGGAAATACTCACTGTTCTTTCAGTGTTTTC | 2417 |
| | AAAAACTTCTCGTGCTTCTTCAAAACTACACTTTTCTTCCATAC | |
| | ATTCTCTCTCAAGGTTCCCTTGAACAAACTCTTC | |
| | GTTTTGAAGAAGCACGA | 2418 |
| | TCGTGCTTCTTCAAAAC | 2419 |
| Haemophilia B | AAGAGTTTGTTCAAGGGAACCTTGAGAGAGAATGTATGGAAG | 2420 |
| Glu27Val | AAAAGTGTAGTTTTGAAGAAGCACGAGAAGTTTTTGAAAACAC | |
| GAA-GTA | TGAAAGAACAGTGAGTATTTCCACATAATACCCTTC | |
| | GAAGGGTATTATGTGGAAATACTCACTGTTCTTTCAGTGTTTT | 2421 |
| | CAAAAACTTCTCGTGCTTCTTCAAAACTACACTTTTCTTCCATA | |
| | CATTCTCTCTCAAGGTTCCCTTGAACAAACTCTT | |
| | TTTTGAAGAAGCACGAG | 2422 |
| | CTCGTGCTTCTTCAAAA | 2423 |
| Haemophilia B | TTGTTCAAGGGAACCTTGAGAGAGAATGTATGGAAGAAAAGT | 2424 |
| Arg29Gln | GTAGTTTTGAAGAAGCACGAGAAGTTTTTGAAAACACTGAAAG | |
| CGA-CAA | AACAGTGAGTATTTCCACATAATACCCTTCAGATGC | |
| | GCATCTGAAGGGTATTATGTGGAAATACTCACTGTTCTTTCAG | 2425 |
| | TGTTTTCAAAAACTTCTCGTGCTTCTTCAAAACTACACTTTTCT | |
| | TCCATACATTCTCTCTCAAGGTTCCCTTGAACAA | |
| | AGAAGCACGAGAAGTTT | 2426 |
| | AAACTTCTCGTGCTTCT | 2427 |
| Haemophilia B | TTGTTCAAGGGAACCTTGAGAGAGAATGTATGGAAGAAAAGT | 2428 |
| Arg29Pro | GTAGTTTTGAAGAAGCACGAGAAGTTTTTGAAAACACTGAAAG | |
| CGA-CCA | AACAGTGAGTATTTCCACATAATACCCTTCAGATGC | |
| | GCATCTGAAGGGTATTATGTGGAAATACTCACTGTTCTTTCAG | 2429 |
| | TGTTTTCAAAAACTTCTCGTGCTTCTTCAAAACTACACTTTTCT | |
| | TCCATACATTCTCTCTCAAGGTTCCCTTGAACAA | |
| | AGAAGCACGAGAAGTTT | 2430 |
| | AAACTTCTCGTGCTTCT | 2431 |
| Haemophilia B | TTTGTTCAAGGGAACCTTGAGAGAGAATGTATGGAAGAATAAG | 2432 |
| Arg29Term | TGTAGTTTTGAAGAAGCACGAGAAGTTTTTGAAAACACTGAAA | |
| aCGA-TGA | GAACAGTGAGTATTTCCACATAATACCCTTCAGATG | |
| | CATCTGAAGGGTATTATGTGGAAATACTCACTGTTCTTTCAGT | 2433 |
| | GTTTTCAAAAACTTCTCGTGCTTCTTCAAAACTACACTTTTCTT | |
| | CCATACATTCTCTCTCAAGGTTCCCTTGAACAAA | |
| | AAGAAGCACGAGAAGTT | 2434 |
| | AACTTCTCGTGCTTCTT | 2435 |
| Haemophilia B | GTTCAAGGGAACCTTGAGAGAGAATGTATGGAAGAAAAGTGT | 2436 |
| Glu30Lys | AGTTTTGAAGAAGCACGAGAAGTTTTTGAAAACACTGAAAGAA | |
| aGAA-AAA | CAGTGAGTATTTCCACATAATACCCTTCAGATGCAG | |
| | CTGCATCTGAAGGGTATTATGTGGAAATACTCACTGTTCTTTC | 2437 |
| | AGTGTTTTCAAAAACTTCTCGTGCTTCTTCAAAACTACACTTTT | |
| | CTTCCATACATTCTCTCTCAAGGTTCCCTTGAAC | |
| | AAGCACGAGAAGTTTTT | 2438 |
| | AAAAACTTCTCGTGCTT | 2439 |
| Haemophilia B | GTTCAAGGGAACCTTGAGAGAGAATGTATGGAAGAAAAGTGT | 2440 |
| Glu30Term | AGTTTTGAAGAAGCACGAGAAGTTTTTGAAAACACTGAAAGAA | |
| aGAA-TAA | CAGTGAGTATTTCCACATAATACCCTTCAGATGCAG | |
| | CTGCATCTGAAGGGTATTATGTGGAAATACTCACTGTTCTTTC | 2441 |
| | AGTGTTTTCAAAAACTTCTCGTGCTTCTTCAAAACTACACTTTT | |
| | CTTCCATACATTCTCTCTCAAGGTTCCCTTGAAC | |
| | AAGCACGAGAAGTTTTT | 2442 |
| | AAAAACTTCTCGTGCTT | 2443 |
| Haemophilia B | CCTTGAGAGAGAATGTATGGAAGAAAAGTGTAGTTTTGAAGA | 2444 |
| Glu33Asp | AGCACGAGAAGTTTTTGAAAACACTGAAAGAACAGTGAGTAT | |
| GAAa-GAC | TTCCACATAATACCCTTCAGATGCAGAGCATAGAATA | |
| | TATTCTATGCTCTGCATCTGAAGGGTATTATGTGGAAATACTC | 2445 |
| | ACTGTTCTTTCAGTGTTTTCAAAAACTTCTCGTGCTTCTTCAAA | |
| | ACTACACTTTTCTTCCATACATTCTCTCTCAAGG | |
| | GTTTTTGAAAACACTGA | 2446 |
| | TCAGTGTTTTCAAAAAC | 2447 |
| Haemophilia B | AACCTTGAGAGAGAATGTATGGAAGAAAAGTGTAGTTTTGAA | 2448 |
| Glu33Term | GAAGCACGAGAAGTTTTTGAAAACACTGAAAGAACAGTGAGT | |
| tGAA-TAA | ATTTCCACATAATACCCTTCAGATGCAGAGCATAGAA | |
| | TTCTATGCTCTGCATCTGAAGGGTATTATGTGGAAATACTCAC | 2449 |
| | TGTTCTTTCAGTGTTTTCAAAAACTTCTCGTGCTTCTTCAAAAC | |
| | TACACTTTTCTTCCATACATTCTCTCTCAAGGTT | |
| | AAGTTTTTGAAAACACT | 2450 |
| | AGTGTTTTCAAAAACTT | 2451 |
| Haemophilia B | CAAAACACTTTAGATATTACCGTTAATTTGTCTTCTTTTATTCTT | 2452 |
| Trp42Term | TATAGACTGAATTTTGGAAGCAGTATGTTGGTAAGCAATTCAT | |
| TGG-TAG | TTTATCCTCTAGCTAATATATGAAACATATGAG | |
| | CTCATATGTTTCATATATTAGCTAGAGGATAAAATGAATTGCTT | 2453 |
| | ACCAACATACTGCTTCCAAAATTCAGTCTATAAAGAATAAAAG | |
| | AAGACAAATTAACGGTAATATCTAAAGTGTTTTG | |
| | TGAATTTTGGAAGCAGT | 2454 |
| | ACTGCTTCCAAAATTCA | 2455 |
| Haemophilia B | AAACACTTTAGATATTACCGTTAATTTGTCTTCTTTTATTCTTTA | 2456 |
| Lys43Glu | TAGACTGAATTTTGGAAGCAGTATGTTGGTAAGCAATTCATTT | |
| gAAG-GAG | TATCCTCTAGCTAATATATGAAACATATGAGAA | |
| | TTCTCATATGTTTCATATATTAGCTAGAGGATAAAATGAATTGC | 2457 |
| | TTACCAACATACTGCTTCCAAAATTCAGTCTATAAAGAATAAAA | |
| | GAAGACAAATTAACGGTAATATCTAAAGTGTTT | |
| | AATTTTGGAAGCAGTAT | 2458 |
| | ATACTGCTTCCAAAATT | 2459 |
| Haemophilia B | CACTTTAGATATTACCGTTAATTTGTCTTCTTTTATTCTTTATAG | 2460 |
| Gln44Term | ACTGAATTTTGGAAGCAGTATGTTGGTAAGCAATTCATTTTAT | |
| gCAG-TAG | CCTCTAGCTAATATATGAAACATATGAGAATTA | |
| | TAATTCTCATATGTTTCATATATTAGCTAGAGGATAAAATGAAT | 2461 |
| | TGCTTACCAACATACTGCTTCCAAAATTCAGTCTATAAAGAAT | |
| | AAAAGAAGACAAATTAACGGTAATATCTAAAGTG | |
| | TTTGGAAGCAGTATGTT | 2462 |
| | AACATACTGCTTCCAAA | 2463 |
| Haemophilia B | CCGGGCATTCTAAGCAGTTTACGTGCCAATTCAATTTCTTAAC | 2464 |
| Asp49Gly | CTATCTCAAAGATGGAGATCAGTGTGAGTCCAATCCATGTTTA | |
| GAT-GGT | AATGGCGGCAGTTGCAAGGATGACATTAATTCCTA | |
| | TAGGAATTAATGTCATCCTTGCAACTGCCGCCATTTAAACATG | 2465 |
| | GATTGGACTCACACTGATCTCCATCTTTGAGATAGGTTAAGAA | |
| | ATTGAATTGGCACGTAAACTGCTTAGAATGCCCGG | |
| | AGATGGAGATCAGTGTG | 2466 |
| | CACACTGATCTCCATCT | 2467 |
| Haemophilia B | GCATTCTAAGCAGTTTACGTGCCAATTCAATTTCTTAACCTAT | 2468 |
| Gln50His | CTCAAAGATGGAGATCAGTGTGAGTCCAATCCATGTTTAAATG | |
| CAGt-CAC | GCGGCAGTTGCAAGGATGACATTAATTCCTATGAA | |
| | TTCATAGGAATTAATGTCATCCTTGCAACTGCCGCCATTTAAA | 2469 |
| | CATGGATTGGACTCACACTGATCTCCATCTTTGAGATAGGTTA | |
| | AGAAATTGAATTGGCACGTAAACTGCTTAGAATGC | |
| | GGAGATCAGTGTGAGTC | 2470 |
| | GACTCACACTGATCTCC | 2471 |
| Haemophilia B | GGCATTCTAAGCAGTTTACGTGCCAATTCAATTTCTTAACCTA | 2472 |
| Gln50Pro | TCTCAAAGATGGAGATCAGTGTGAGTCCAATCCATGTTTAAAT | |
| CAG-CCG | GGCGGCAGTTGCAAGGATGACATTAATTCCTATGA | |
| | TCATAGGAATTAATGTCATCCTTGCAACTGCCGCCATTTAAAC | 2473 |
| | ATGGATTGGACTCACACTGATCTCCATCTTTGAGATAGGTTAA | |
| | GAAATTGAATTGGCACGTAAACTGCTTAGAATGCC | |
| | TGGAGATCAGTGTGAGT | 2474 |
| | ACTCACACTGATCTCCA | 2475 |
| Haemophilia B | GGGCATTCTAAGCAGTTTACGTGCCAATTCAATTTCTTAACCT | 2476 |
| Gln50Term | ATCTCAAAGATGGAGATCAGTGTGAGTCCAATCCATGTTTAAA | |
| tCAG-TAG | TGGCGGCAGTTGCAAGGATGACATTAATTCCTATG | |
| tCAG-TAG | CATAGGAATTAATGTCATCCTTGCAACTGCCGCCATTTAAACA | 2477 |
| | TGGATTGGACTCACACTGATCTCCATCTTTGAGATAGGTTAAG | |
| | AAATTGAATTGGCACGTAAACTGCTTAGAATGCCC | |
| | ATGGAGATCAGTGTGAG | 2478 |
| | CTCACACTGATCTCCAT | 2479 |
| Haemophilia B | CATTCTAAGCAGTTTACGTGCCAATTCAATTTCTTAACCTATCT | 2480 |
| Cys51Arg | CAAAGATGGAGATCAGTGTGAGTCCAATCCATGTTTAAATGG | |
| gTGT-CGT | CGGCAGTTGCAAGGATGACATTAATTCCTATGAAT | |
| | ATTCATAGGAATTAATGTCATCCTTGCAACTGCCGCCATTTAA | 2481 |
| | ACATGGATTGGACTCACACTGATCTCCATCTTTGAGATAGGTT | |
| | AAGAAATTGAATTGGCACGTAAACTGCTTAGAATG | |
| | GAGATCAGTGTGAGTCC | 2482 |
| | GGACTCACACTGATCTC | 2483 |
| Haemophilia B | CATTCTAAGCAGTTTACGTGCCAATTCAATTTCTTAACCTATCT | 2484 |
| Cys51Ser | CAAAGATGGAGATCAGTGTGAGTCCAATCCATGTTTAAATGG | |
| gTGT-AGT | CGGCAGTTGCAAGGATGACATTAATTCCTATGAAT | |
| | ATTCATAGGAATTAATGTCATCCTTGCAACTGCCGCCATTTAA | 2485 |
| | ACATGGATTGGACTCACACTGATCTCCATCTTTGAGATAGGTT | |
| | AAGAAATTGAATTGGCACGTAAACTGCTTAGAATG | |
| | GAGATCAGTGTGAGTCC | 2486 |
| | GGACTCACACTGATCTC | 2487 |
| Haemophilia B | TTCTAAGCAGTTTACGTGCCAATTCAATTTCTTAACCTATCTCA | 2488 |
| Cys51Trp | AAGATGGAGATCAGTGTGAGTCCAATCCATGTTTAAATGGCG | |
| TGTg-TGG | GCAGTTGCAAGGATGACATTAATTCCTATGAATGT | |
| | ACATTCATAGGAATTAATGTCATCCTTGCAACTGCCGCCATTT | 2489 |
| | AAACATGGATTGGACTCACACTGATCTCCATCTTTGAGATAGG | |
| | TTAAGAAATTGAATTGGCACGTAAACTGCTTAGAA | |
| | GATCAGTGTGAGTCCAA | 2490 |
| | TTGGACTCACACTGATC | 2491 |
| Haemophilia B | TCTAAGCAGTTTACGTGCCAATTCAATTTCTTAACCTATCTCAA | 2492 |
| Glu52Term | AGATGGAGATCAGTGTGAGTCCAATCCATGTTTAAATGGCGG | |
| tGAG-TAG | CAGTTGCAAGGATGACATTAATTCCTATGAATGTT | |
| | AACATTCATAGGAATTAATGTCATCCTTGCAACTGCCGCCATT | 2493 |
| | TAAACATGGATTGGACTCACACTGATCTCCATCTTTGAGATAG | |
| | GTTAAGAAATTGAATTGGCACGTAAACTGCTTAGA | |
| | ATCAGTGTGAGTCCAAT | 2494 |
| | ATTGGACTCACACTGAT | 2495 |
| Haemophilia B | TTTACGTGCCAATTCAATTTCTTAACCTATCTCAAAGATGGAG | 2496 |
| Pro55Ala | ATCAGTGTGAGTCCAATCCATGTTTAAATGGCGGCAGTTGCA | |
| tCCA-GCA | AGGATGACATTAATTCCTATGAATGTTGGTGTCCCT | |
| | AGGGACACCAACATTCATAGGAATTAATGTCATCCTTGCAACT | 2497 |
| | GCCGCCATTTAAACATGGATTGGACTCACACTGATCTCCATCT | |
| | TTGAGATAGGTTAAGAAATTGAATTGGCACGTAAA | |
| | AGTCCAATCCATGTTTA | 2498 |
| | TAAACATGGATTGGACT | 2499 |
| Haemophilia B | TTACGTGCCAATTCAATTTCTTAACCTATCTCAAAGATGGAGA | 2500 |
| Pro55Arg | TCAGTGTGAGTCCAATCCATGTTTAAATGGCGGCAGTTGCAA | |
| CCA-CGA | GGATGACATTAATTCCTATGAATGTTGGTGTCCCTT | |
| | AAGGGACACCAACATTCATAGGAATTAATGTCATCCTTGCAAC | 2501 |
| | TGCCGCCATTTAAACATGGATTGGACTCACACTGATCTCCATC | |
| | TTTGAGATAGGTTAAGAAATTGAATTGGCACGTAA | |
| | GTCCAATCCATGTTTAA | 2502 |
| | TTAAACATGGATTGGAC | 2503 |
| Haemophilia B | TTACGTGCCAATTCAATTTCTTAACCTATCTCAAAGATGGAGA | 2504 |
| Pro55Gln | TCAGTGTGAGTCCAATCCATGTTTAAATGGCGGCAGTTGCAA | |
| CCA-CAA | GGATGACATTAATTCCTATGAATGTTGGTGTCCCTT | |
| | AAGGGACACCAACATTCATAGGAATTAATGTCATCCTTGCAAC | 2505 |
| | TGCCGCCATTTAAACATGGATTGGACTCACACTGATCTCCATC | |
| | TTTGAGATAGGTTAAGAAATTGAATTGGCACGTAA | |
| | GTCCAATCCATGTTTAA | 2506 |
| | TTAAACATGGATTGGAC | 2507 |
| Haemophilia B | TTACGTGCCAATTCAATTTCTTAACCTATCTCAAAGATGGAGA | 2508 |
| Pro55Leu | TCAGTGTGAGTCCAATCCATGTTTAAATGGCGGCAGTTGCAA | |
| CCA-CTA | GGATGACATTAATTCCTATGAATGTTGGTGTCCCTT | |
| | AAGGGACACCAACATTCATAGGAATTAATGTCATCCTTGCAAC | 2509 |
| | TGCCGCCATTTAAACATGGATTGGACTCACACTGATCTCCATC | |
| | TTTGAGATAGGTTAAGAAATTGAATTGGCACGTAA | |
| | GTCCAATCCATGTTTAA | 2510 |
| | TTAAACATGGATTGGAC | 2511 |
| Haemophilia B | TTTACGTGCCAATTCAATTTCTTAACCTATCTCAAAGATGGAG | 2512 |
| Pro55Ser | ATCAGTGTGAGTCCAATCCATGTTTAAATGGCGGCAGTTGCA | |
| tCCA-TCA | AGGATGACATTAATTCCTATGAATGTTGGTGTCCCT | |
| | AGGGACACCAACATTCATAGGAATTAATGTCATCCTTGCAACT | 2513 |
| | GCCGCCATTTAAACATGGATTGGACTCACACTGATCTCCATCT | |
| | TTGAGATAGGTTAAGAAATTGAATTGGCACGTAAA | |
| | AGTCCAATCCATGTTTA | 2514 |
| | TAAACATGGATTGGACT | 2515 |
| Haemophilia B | ACGTGCCAATTCAATTTCTTAACCTATCTCAAAGATGGAGATC | 2516 |
| Cys56Arg | AGTGTGAGTCCAATCCATGTTTAAATGGCGGCAGTTGCAAGG | |
| aTGT-CGT | ATGACATTAATTCCTATGAATGTTGGTGTCCCTTTG | |
| | CAAAGGGACACCAACATTCATAGGAATTAATGTCATCCTTGCA 2517 | |
| | ACTGCCGCCATTTAAACATGGATTGGACTCACACTGATCTCC | |
| | ATCTTTGAGATAGGTTAAGAAATTGAATTGGCACGT | |
| | CCAATCCATGTTTAAAT | 2518 |
| | ATTTAAACATGGATTGG | 2519 |
| Haemophilia B | ACGTGCCAATTCAATTTCTTAACCTATCTCAAAGATGGAGATC | 2520 |
| Cys56Ser | AGTGTGAGTCCAATCCATGTTTAAATGGCGGCAGTTGCAAGG | |
| aTGT-AGT | ATGACATTAATTCCTATGAATGTTGGTGTCCCTTTG | |
| | CAAAGGGACACCAACATTCATAGGAATTAATGTCATCCTTGCA | 2521 |
| | ACTGCCGCCATTTAAACATGGATTGGACTCACACTGATCTCC | |
| | ATCTTTGAGATAGGTTAAGAAATTGAATTGGCACGT | |
| | CCAATCCATGTTTAAAT | 2522 |
| | ATTTAAACATGGATTGG | 2523 |
| Haemophilia B | CGTGCCAATTCAATTTCTTAACCTATCTCAAAGATGGAGATCA | 2524 |
| Cys56Ser | GTGTGAGTCCAATCCATGTTTAAATGGCGGCAGTTGCAAGGA | |
| TGT-TCT | TGACATTAATTCCTATGAATGTTGGTGTCCCTTTGG | |
| | CCAAAGGGACACCAACATTCATAGGAATTAATGTCATCCTTGC | 2525 |
| | AACTGCCGCCATTTAAACATGGATTGGACTCACACTGATCTCC | |
| | ATCTTTGAGATAGGTTAAGAAATTGAATTGGCACG | |
| | CAATCCATGTTTAAATG | 2526 |
| | CATTTAAACATGGATTG | 2527 |
| Haemophilia B | CGTGCCAATTCAATTTCTTAACCTATCTCAAAGATGGAGATCA | 2528 |
| Cys56Tyr | GTGTGAGTCCAATCCATGTTTAAATGGCGGCAGTTGCAAGGA | |
| TGT-TAT | TGACATTAATTCCTATGAATGTTGGTGTCCCTTTGG | |
| | CCAAAGGGACACCAACATTCATAGGAATTAATGTCATCCTTGC | 2529 |
| | AACTGCCGCCATTTAAACATGGATTGGACTCACACTGATCTCC | |
| | ATCTTTGAGATAGGTTAAGAAATTGAATTGGCACG | |
| | CAATCCATGTTTAAATG | 2530 |
| | CATTTAAACATGGATTG | 2531 |
| Haemophilia B | ATTCAATTTCTTAACCTATCTCAAAGATGGAGATCAGTGTGAG | 2532 |
| Asn58Lys | TCCAATCCATGTTTAAATGGCGGCAGTTGCAAGGATGACATTA | |
| AATg-AAG | ATTCCTATGAATGTTGGTGTCCCTTTGGATTTGAA | |
| | TTCAAATCCAAAGGGACACCAACATTCATAGGAATTAATGTCA | 2533 |
| | TCCTTGCAACTGCCGCCATTTAAACATGGATTGGACTCACACT | |
| | GATCTCCATCTTTGAGATAGGTTAAGAAATTGAAT | |
| | TGTTTAAATGGCGGCAG | 2534 |
| | CTGCCGCCATTTAAACA | 2535 |
| Haemophilia B | TCAATTTCTTAACCTATCTCAAAGATGGAGATCAGTGTGAGTC | 2536 |
| Gly59Asp | CAATCCATGTTTAAATGGCGGCAGTTGCAAGGATGACATTAAT | |
| GGC-GAC | TCCTATGAATGTTGGTGTCCCTTTGGATTTGAAGG | |
| | CCTTCAAATCCAAAGGGACACCAACATTCATAGGAATTAATGT | 2537 |
| | CATCCTTGCAACTGCCGCCATTTAAACATGGATTGGACTCACA | |
| | CTGATCTCCATCTTTGAGATAGGTTAAGAAATTGA | |
| | TTTAAATGGCGGCAGTT | 2538 |
| | AACTGCCGCCATTTAAA | 2539 |
| Haemophilia B | TCAATTTCTTAACCTATCTCAAAGATGGAGATCAGTGTGAGTC | 2540 |
| Gly59Val | CAATCCATGTTTAAATGGCGGCAGTTGCAAGGATGACATTAAT | |
| GGC-GTC | TCCTATGAATGTTGGTGTCCCTTTGGATTTGAAGG | |
| | CCTTCAAATCCAAAGGGACACCAACATTCATAGGAATTAATGT | 2541 |
| | CATCCTTGCAACTGCCGCCATTTAAACATGGATTGGACTCACA | |
| | CTGATCTCCATCTTTGAGATAGGTTAAGAAATTGA | |
| | TTTAAATGGCGGCAGTT | 2542 |
| | AACTGCCGCCATTTAAA | 2543 |
| Haemophilia B | TTCAATTTCTTAACCTATCTCAAAGATGGAGATCAGTGTGAGT | 2544 |
| Gly59Ser | CCAATCCATGTTTAAATGGCGGCAGTTGCAAGGATGACATTA | |
| tGGC-AGC | ATTCCTATGAATGTTGGTGTCCCTTTGGATTTGAAG | |
| | CTTCAAATCCAAAGGGACACCAACATTCATAGGAATTAATGTC | 2545 |
| | ATCCTTGCAACTGCCGCCATTTAAACATGGATTGGACTCACAC | |
| | TGATCTCCATCTTTGAGATAGGTTAAGAAATTGAA | |
| | GTTTAAATGGCGGCAGT | 2546 |
| | ACTGCCGCCATTTAAAC | 2547 |
| Haemophilia B | AATTTCTTAACCTATCTCAAAGATGGAGATCAGTGTGAGTCCA | 2548 |
| Gly60Ser | ATCCATGTTTAAATGGCGGCAGTTGCAAGGATGACATTAATTC | |
| cGGC-AGC | CTATGAATGTTGGTGTCCCTTTGGATTTGAAGGAA | |
| | TTCCTTCAAATCCAAAGGGACACCAACATTCATAGGAATTAAT | 2549 |
| | GTCATCCTTGCAACTGCCGCCATTTAAACATGGATTGGACTCA | |
| | CACTGATCTCCATCTTTGAGATAGGTTAAGAAATT | |
| | TAAATGGCGGCAGTTGC | 2550 |
| | GCAACTGCCGCCATTTA | 2551 |
| Haemophilia B | AATTTCTTAACCTATCTCAAAGATGGAGATCAGTGTGAGTCCA | 2552 |
| Gly60Cys | ATCCATGTTTAAATGGCGGCAGTTGCAAGGATGACATTAATTC | |
| cGGC-TGC | CTATGAATGTTGGTGTCCCTTTGGATTTGAAGGAA | |
| | TTCCTTCAAATCCAAAGGGACACCAACATTCATAGGAATTAAT | 2553 |
| | GTCATCCTTGCAACTGCCGCCATTTAAACATGGATTGGACTCA | |
| | CACTGATCTCCATCTTTGAGATAGGTTAAGAAATT | |
| | TAAATGGCGGCAGTTGC | 2554 |
| | GCAACTGCCGCCATTTA | 2555 |
| Haemophilia B | ATTTCTTAACCTATCTCAAAGATGGAGATCAGTGTGAGTCCAA | 2556 |
| Gly60Asp | TCCATGTTTAAATGGCGGCAGTTGCAAGGATGACATTAATTCC | |
| GGC-GAC | TATGAATGTTGGTGTCCCTTTGGATTTGAAGGAAA | |
| | TTTCCTTCAAATCCAAAGGGACACCAACATTCATAGGAATTAA | 2557 |
| | TGTCATCCTTGCAACTGCCGCCATTTAAACATGGATTGGACTC | |
| | ACACTGATCTCCATCTTTGAGATAGGTTAAGAAAT | |
| | AAATGGCGGCAGTTGCA | 2558 |
| | TGCAACTGCCGCCATTT | 2559 |
| Haemophilia B | AATTTCTTAACCTATCTCAAAGATGGAGATCAGTGTGAGTCCA | 2560 |
| Gly60Arg | ATCCATGTTTAAATGGCGGCAGTTGCAAGGATGACATTAATTC | |
| cGGC-CGC | CTATGAATGTTGGTGTCCCTTTGGATTTGAAGGAA | |
| | TTCCTTCAAATCCAAAGGGACACCAACATTCATAGGAATTAAT | 2561 |
| | GTCATCCTTGCAACTGCCGCCATTTAAACATGGATTGGACTCA | |
| | CACTGATCTCCATCTTTGAGATAGGTTAAGAAATT | |
| | TAAATGGCGGCAGTTGC | 2562 |
| | GCAACTGCCGCCATTTA | 2563 |
| Haemophilia B | TAACCTATCTCAAAGATGGAGATCAGTGTGAGTCCAATCCATG | 2564 |
| Cys62Tyr | TTTAAATGGCGGCAGTTGCAAGGATGACATTAATTCCTATGAA | |
| TGC-TAC | TGTTGGTGTCCCTTTGGATTTGAAGGAAAGAACTG | |
| | CAGTTCTTTCCTTCAAATCCAAAGGGACACCAACATTCATAGG | 2565 |
| | AATTAATGTCATCCTTGCAACTGCCGCCATTTAAACATGGATT | |
| | GGACTCACACTGATCTCCATCTTTGAGATAGGTTA | |
| | CGGCAGTTGCAAGGATG | 2566 |
| | CATCCTTGCAACTGCCG | 2567 |
| Haemophilia B | TAACCTATCTCAAAGATGGAGATCAGTGTGAGTCCAATCCATG | 2568 |
| Cys62Ser | TTTAAATGGCGGCAGTTGCAAGGATGACATTAATTCCTATGAA | |
| TGC-TCC | TGTTGGTGTCCCTTTGGATTTGAAGGAAAGAACTG | |
| | CAGTTCTTTCCTTCAAATCCAAAGGGACACCAACATTCATAGG | 2569 |
| | AATTAATGTCATCCTTGCAACTGCCGCCATTTAAACATGGATT | |
| | GGACTCACACTGATCTCCATCTTTGAGATAGGTTA | |
| | CGGCAGTTGCAAGGATG | 2570 |
| | CATCCTTGCAACTGCCG | 2571 |
| Haemophilia B | AACCTATCTCAAAGATGGAGATCAGTGTGAGTCCAATCCATGT | 2572 |
| Cys62Term | TTAAATGGCGGCAGTTGCAAGGATGACATTAATTCCTATGAAT | |
| TGCa-TGA | GTTGGTGTCCCTTTGGATTTGAAGGAAAGAACTGT | |
| | ACAGTTCTTTCCTTCAAATCCAAAGGGACACCAACATTCATAG | 2573 |
| | GAATTAATGTCATCCTTGCAACTGCCGCCATTTAAACATGGAT | |
| | TGGACTCACACTGATCTCCATCTTTGAGATAGGTT | |
| | GGCAGTTGCAAGGATGA | 2574 |
| | TCATCCTTGCAACTGCC | 2575 |
| Haemophilia B | TCTCAAAGATGGAGATCAGTGTGAGTCCAATCCATGTTTAAAT | 2576 |
| Asp64Glu | GGCGGCAGTTGCAAGGATGACATTAATTCCTATGAATGTTGG | |
| GATg-GAG | TGTCCCTTTGGATTTGAAGGAAAGAACTGTGAATTA | |
| | TAATTCACAGTTCTTTCCTTCAAATCCAAAGGGACACCAACAT | 2577 |
| | TCATAGGAATTAATGTCATCCTTGCAACTGCCGCCATTTAAAC | |
| | ATGGATTGGACTCACACTGATCTCCATCTTTGAGA | |
| | TGCAAGGATGACATTAA | 2578 |
| | TTAATGTCATCCTTGCA | 2579 |
| Haemophilia B | ATCTCAAAGATGGAGATCAGTGTGAGTCCAATCCATGTTTAAA | 2580 |
| Asp64Gly | TGGCGGCAGTTGCAAGGATGACATTAATTCCTATGAATGTTG | |
| GAT-GGT | GTGTCCCTTTGGATTTGAAGGAAAGAACTGTGAATT | |
| | AATTCACAGTTCTTTCCTTCAAATCCAAAGGGACACCAACATT | 2581 |
| | CATAGGAATTAATGTCATCCTTGCAACTGCCGCCATTTAAACA | |
| | TGGATTGGACTCACACTGATCTCCATCTTTGAGAT | |
| | TTGCAAGGATGACATTA | 2582 |
| | TAATGTCATCCTTGCAA | 2583 |
| Haemophilia B | TATCTCAAAGATGGAGATCAGTGTGAGTCCAATCCATGTTTAA | 2584 |
| Asp64Asn | ATGGCGGCAGTTGCAAGGATGACATTAATTCCTATGAATGTT | |
| gGAT-AAT | GGTGTCCCTTTGGATTTGAAGGAAAGAACTGTGAAT | |
| | ATTCACAGTTCTTTCCTTCAAATCCAAAGGGACACCAACATTC | 2585 |
| | ATAGGAATTAATGTCATCCTTGCAACTGCCGCCATTTAAACAT | |
| | GGATTGGACTCACACTGATCTCCATCTTTGAGATA | |
| | GTTGCAAGGATGACATT | 2586 |
| | AATGTCATCCTTGCAAC | 2587 |
| Haemophilia B | AAGATGGAGATCAGTGTGAGTCCAATCCATGTTTAAATGGCG | 2588 |
| Ile66Ser | GCAGTTGCAAGGATGACATTAATTCCTATGAATGTTGGTGTCC | |
| ATT-AGT | CTTTGGATTTGAAGGAAAGAACTGTGAATTAGGTAA | |
| | TTACCTAATTCACAGTTCTTTCCTTCAAATCCAAAGGGACACC | 2589 |
| | AACATTCATAGGAATTAATGTCATCCTTGCAACTGCCGCCATT | |
| | TAAACATGGATTGGACTCACACTGATCTCCATCTT | |
| | GGATGACATTAATTCCT | 2590 |
| | AGGAATTAATGTCATCC | 2591 |
| Haemophilia B | AAGATGGAGATCAGTGTGAGTCCAATCCATGTTTAAATGGCG | 2592 |
| Ile66Thr | GCAGTTGCAAGGATGACATTAATTCCTATGAATGTTGGTGTCC | |
| ATT-ACT | CTTTGGATTTGAAGGAAAGAACTGTGAATTAGGTAA | |
| | TTACCTAATTCACAGTTCTTTCCTTCAAATCCAAAGGGACACC | 2593 |
| | AACATTCATAGGAATTAATGTCATCCTTGCAACTGCCGCCATT | |
| | TAAACATGGATTGGACTCACACTGATCTCCATCTT | |
| | GGATGACATTAATTCCT | 2594 |
| | AGGAATTAATGTCATCC | 2595 |
| Haemophilia B | TGGAGATCAGTGTGAGTCCAATCCATGTTTAAATGGCGGCAG | 2596 |
| Asn67Lys | TTGCAAGGATGACATTAATTCCTATGAATGTTGGTGTCCCTTT | |
| AATt-AAA | GGATTTGAAGGAAAGAACTGTGAATTAGGTAAGTAA | |
| | TTACTTACCTAATTCACAGTTCTTTCCTTCAAATCCAAAGGGA | 2597 |
| | CACCAACATTCATAGGAATTAATGTCATCCTTGCAACTGCCGC | |
| | CATTTAAACATGGATTGGACTCACACTGATCTCCA | |
| | GACATTAATTCCTATGA | 2598 |
| | TCATAGGAATTAATGTC | 2599 |
| Haemophilia B | ATCAGTGTGAGTCCAATCCATGTTTAAATGGCGGCAGTTGCA | 2600 |
| Tyr69Cys | AGGATGACATTAATTCCTATGAATGTTGGTGTCCCTTTGGATT | |
| TAT-TGT | TGAAGGAAAGAACTGTGAATTAGGTAAGTAACTATT | |
| | AATAGTTACTTACCTAATTCACAGTTCTTTCCTTCAAATCCAAA | 2601 |
| | GGGACACCAACATTCATAGGAATTAATGTCATCCTTGCAACTG | |
| | CCGCCATTTAAACATGGATTGGACTCACACTGAT | |
| | TAATTCCTATGAATGTT | 2602 |
| | AACATTCATAGGAATTA | 2603 |
| Haemophilia B | TGAGTCCAATCCATGTTTAAATGGCGGCAGTTGCAAGGATGA | 2604 |
| Cys71Term | CATTAATTCCTATGAATGTTGGTGTCCCTTTGGATTTGAAGGA | |
| TGTt-TGA | AAGAACTGTGAATTAGGTAAGTAACTATTTTTTGAA | |
| | TTCAAAAAATAGTTACTTACCTAATTCACAGTTCTTTCCTTCAA | 2605 |
| | ATCCAAAGGGACACCAACATTCATAGGAATTAATGTCATCCTT | |
| | GCAACTGCCGCCATTTAAACATGGATTGGACTCA | |
| | TATGAATGTTGGTGTCC | 2606 |
| | GGACACCAACATTCATA | 2607 |
| Haemophilia B | GTGAGTCCAATCCATGTTTAAATGGCGGCAGTTGCAAGGATG | 2608 |
| Cys71Ser | ACATTAATTCCTATGAATGTTGGTGTCCCTTTGGATTTGAAGG | |
| TGT-TCT | AAAGAACTGTGAATTAGGTAAGTAACTATTTTTTGA | |
| | TCAAAAAATAGTTACTTACCTAATTCACAGTTCTTTCCTTCAAA | 2609 |
| | TCCAAAGGGACACCAACATTCATAGGAATTAATGTCATCCTTG | |
| | CAACTGCCGCCATTTAAACATGGATTGGACTCAC | |
| | CTATGAATGTTGGTGTC | 2610 |
| | GACACCAACATTCATAG | 2611 |
| Haemophilia B | GTGAGTCCAATCCATGTTTAAATGGCGGCAGTTGCAAGGATG | 2612 |
| Cys71Tyr | ACATTAATTCCTATGAATGTTGGTGTCCCTTTGGATTTGAAGG | |
| TGT-TAT | AAAGAACTGTGAATTAGGTAAGTAACTATTTTTTGA | |
| | TCAAAAAATAGTTACTTACCTAATTCACAGTTCTTTCCTTCAAA | 2613 |
| | TCCAAAGGGACACCAACATTCATAGGAATTAATGTCATCCTTG | |
| | CAACTGCCGCCATTTAAACATGGATTGGACTCAC | |
| | CTATGAATGTTGGTGTC | 2614 |
| | GACACCAACATTCATAG | 2615 |
| Haemophilia B | TGTGAGTCCAATCCATGTTTAAATGGCGGCAGTTGCAAGGAT | 2616 |
| Cys11Ser | GACATTAATTCCTATGAATGTTGGTGTCCCTTTGGATTTGAAG | |
| aTGT-AGT | GAAAGAACTGTGAATTAGGTAAGTAACTATTTTTTG | |
| | CAAAAAATAGTTACTTACCTAATTCACAGTTCTTTCCTTCAAAT | 2617 |
| | CCAAAGGGACACCAACATTCATAGGAATTAATGTCATCCTTGC | |
| | AACTGCCGCCATTTAAACATGGATTGGACTCACA | |
| | CCTATGAATGTTGGTGT | 2618 |
| | ACACCAACATTCATAGG | 2619 |
| Haemophilia B | GAGTCCAATCCATGTTTAAATGGCGGCAGTTGCAAGGATGAC | 2620 |
| Trp72Arg | ATTAATTCCTATGAATGTTGGTGTCCCTTTGGATTTGAAGGAA | |
| tTGG-AGG | AGAACTGTGAATTAGGTAAGTAACTATTTTTTGAAT | |
| | ATTCAAAAAATAGTTACTTACCTAATTCACAGTTCTTTCCTTCA | 2621 |
| | AATCCAAAGGGACACCAACATTCATAGGAATTAATGTCATCCT | |
| | TGCAACTGCCGCCATTTAAACATGGATTGGACTC | |
| | ATGAATGTTGGTGTCCC | 2622 |
| | GGGACACCAACATTCAT | 2623 |
| Haemophilia B | GTCCAATCCATGTTTAAATGGCGGCAGTTGCAAGGATGACAT | 2624 |
| Trp72Term | TAATTCCTATGAATGTTGGTGTCCCTTTGGATTTGAAGGAAAG | |
| TGGt-TGA | AACTGTGAATTAGGTAAGTAACTATTTTTTGAATAC | |
| | GTATTCAAAAAATAGTTACTTACCTAATTCACAGTTCTTTCCTT | 2625 |
| | CAAATCCAAAGGGACACCAACATTCATAGGAATTAATGTCATC | |
| | CTTGCAACTGCCGCCATTTAAACATGGATTGGAC | |
| | GAATGTTGGTGTCCCTT | 2626 |
| | AAGGGACACCAACATTC | 2627 |
| Haemophilia B | CCAATCCATGTTTAAATGGCGGCAGTTGCAAGGATGACATTA | 2628 |
| Cys73Tyr | ATTCCTATGAATGTTGGTGTCCCTTTGGATTTGAAGGAAAGAA | |
| TGT-TAT | CTGTGAATTAGGTAAGTAACTATTTTTTGAATACTC | |
| | GAGTATTCAAAAAATAGTTACTTACCTAATTCACAGTTCTTTCC | 2629 |
| | TTCAAATCCAAAGGGACACCAACATTCATAGGAATTAATGTCA | |
| | TCCTTGCAACTGCCGCCATTTAAACATGGATTGG | |
| | ATGTTGGTGTCCCTTTG | 2630 |
| | CAAAGGGACACCAACAT | 2631 |
| Haemophilia B | TCCAATCCATGTTTAAATGGCGGCAGTTGCAAGGATGACATTA | 2632 |
| Cys73Arg | ATTCCTATGAATGTTGGTGTCCCTTTGGATTTGAAGGAAAGAA | |
| gTGT-CGT | CTGTGAATTAGGTAAGTAACTATTTTTTGAATACT | |
| | AGTATTCAAAAAATAGTTACTTACCTAATTCACAGTTCTTTCCT | 2633 |
| | TCAAATCCAAAGGGACACCAACATTCATAGGAATTAATGTCAT | |
| | CCTTGCAACTGCCGCCATTTAAACATGGATTGGA | |
| | AATGTTGGTGTCCCTTT | 2634 |
| | AAAGGGACACCAACATT | 2635 |
| Haemophilia B | CCAATCCATGTTTAAATGGCGGCAGTTGCAAGGATGACATTA | 2636 |
| Cys73Phe | ATTCCTATGAATGTTGGTGTCCCTTTGGATTTGAAGGAAAGAA | |
| TGT-TTT | CTGTGAATTAGGTAAGTAACTATTTTTTGAATACTC | |
| | GAGTATTCAAAAAATAGTTACTTACCTAATTCACAGTTCTTTCC | 2637 |
| | TTCAAATCCAAAGGGACACCAACATTCATAGGAATTAATGTCA | |
| | TCCTTGCAACTGCCGCCATTTAAACATGGATTGG | |
| | ATGTTGGTGTCCCTTTG | 2638 |
| | CAAAGGGACACCAACAT | 2639 |
| Haemophilia B | CAATCCATGTTTAAATGGCGGCAGTTGCAAGGATGACATTAAT | 2640 |
| Cys73Term | TCCTATGAATGTTGGTGTCCCTTTGGATTTGAAGGAAAGAACT | |
| TGTc-TGA | GTGAATTAGGTAAGTAACTATTTTTTGAATACTCA | |
| | TGAGTATTCAAAAAATAGTTACTTACCTAATTCACAGTTCTTTC | 2641 |
| | CTTCAAATCCAAAGGGACACCAACATTCATAGGAATTAATGTC | |
| | ATCCTTGCAACTGCCGCCATTTAAACATGGATTG | |
| | TGTTGGTGTCCCTTTGG | 2642 |
| | CCAAAGGGACACCAACA | 2643 |
| Haemophilia B | GTTTAAATGGCGGCAGTTGCAAGGATGACATTAATTCCTATGA | 2644 |
| Gly76Val | ATGTTGGTGTCCCTTTGGATTTGAAGGAAAGAACTGTGAATTA | |
| GGA-GTA | GGTAAGTAACTATTTTTTGAATACTCATGGTTCAA | |
| | TTGAACCATGAGTATTCAAAAAATAGTTACTTACCTAATTCACA | 2645 |
| | GTTCTTTCCTTCAAATCCAAAGGGACACCAACATTCATAGGAA | |
| | TTAATGTCATCCTTGCAACTGCCGCCATTTAAAC | |
| | TCCCTTTGGATTTGAAG | 2646 |
| | CTTCAAATCCAAAGGGA | 2647 |
| Haemophilia B | TGTTTAAATGGCGGCAGTTGCAAGGATGACATTAATTCCTATG | 2648 |
| Gly76Arg | AATGTTGGTGTCCCTTTGGATTTGAAGGAAAGAACTGTGAATT | |
| tGGA-AGA | AGGTAAGTAACTATTTTTTGAATACTCATGGTTCA | |
| | TGAACCATGAGTATTCAAAAAATAGTTACTTACCTAATTCACA | 2649 |
| | GTTCTTTCCTTCAAATCCAAAGGGACACCAACATTCATAGGAA | |
| | TTAATGTCATCCTTGCAACTGCCGCCATTTAAACA | |
| | GTCCCTTTGGATTTGAA | 2650 |
| | TTCAAATCCAAAGGGAC | 2651 |
| Haemophilia B | TAAATGGCGGCAGTTGCAAGGATGACATTAATTCCTATGAATG | 2652 |
| Phe77Cys | TTGGTGTCCCTTTGGATTTGAAGGAAAGAACTGTGAATTAGGT | |
| TTT-TGT | AAGTAACTATTTTTTGAATACTCATGGTTCAAAGT | |
| | ACTTTGAACCATGAGTATTCAAAAAATAGTTACTTACCTAATTC | 2653 |
| | ACAGTTCTTTCCTTCAAATCCAAAGGGACACCAACATTCATAG | |
| | GAATTAATGTCATCCTTGCAACTGCCGCCATTTA | |
| | CTTTGGATTTGAAGGAA | 2654 |
| | TTCCTTCAAATCCAAAG | 2655 |
| Haemophilia B | TAAATGGCGGCAGTTGCAAGGATGACATTAATTCCTATGAATG | 2656 |
| Phe77Ser | TTGGTGTCCCTTTGGATTTGAAGGAAAGAACTGTGAATTAGGT | |
| TTT-TCT | AAGTAACTATTTTTTGAATACTCATGGTTCAAAGT | |
| | ACTTTGAACCATGAGTATTCAAAAAATAGTTACTTACCTAATTC | 2657 |
| | ACAGTTCTTTCCTTCAAATCCAAAGGGACACCAACATTCATAG | |
| | GAATTAATGTCATCCTTGCAACTGCCGCCATTTA | |
| | CTTTGGATTTGAAGGAA | 2658 |
| | TTCCTTCAAATCCAAAG | 2659 |
| Haemophilia B | TAAATGGCGGCAGTTGCAAGGATGACATTAATTCCTATGAATG | 2660 |
| Phe77Tyr | TTGGTGTCCCTTTGGATTTGAAGGAAAGAACTGTGAATTAGGT | |
| TTT-TAT | AAGTAACTATTTTTTGAATACTCATGGTTCAAAGT | |
| | ACTTTGAACCATGAGTATTCAAAAAATAGTTACTTACCTAATTC | 2661 |
| | ACAGTTCTTTCCTTCAAATCCAAAGGGACACCAACATTCATAG | |
| | GAATTAATGTCATCCTTGCAACTGCCGCCATTTA | |
| | CTTTGGATTTGAAGGAA | 2662 |
| | TTCCTTCAAATCCAAAG | 2663 |
| Haemophilia B | AATGGCGGCAGTTGCAAGGATGACATTAATTCCTATGAATGTT | 2664 |
| Glu78Lys | GGTGTCCCTTTGGATTTGAAGGAAAGAACTGTGAATTAGGTA | |
| tGAA-AAA | AGTAACTATTTTTTGAATACTCATGGTTCAAAGTTT | |
| | AAACTTTGAACCATGAGTATTCAAAAAATAGTTACTTACCTAAT | 2665 |
| | TCACAGTTCTTTCCTTCAAATCCAAAGGGACACCAACATTCAT | |
| | AGGAATTAATGTCATCCTTGCAACTGCCGCCATT | |
| | TTGGATTTGAAGGAAAG | 2666 |
| | CTTTCCTTCAAATCCAA | 2667 |
| Haemophilia B | GCGGCAGTTGCAAGGATGACATTAATTCCTATGAATGTTGGT | 2668 |
| Gly79Val | GTCCCTTTGGATTTGAAGGAAAGAACTGTGAATTAGGTAAGTA | |
| GGA-GTA | ACTATTTTTTGAATACTCATGGTTCAAAGTTTCCCT | |
| | AGGGAAACTTTGAACCATGAGTATTCAAAAAATAGTTACTTAC | 2669 |
| | CTAATTCACAGTTCTTTCCTTCAAATCCAAAGGGACACCAACA | |
| | TTCATAGGAATTAATGTCATCCTTGCAACTGCCGC | |
| | ATTTGAAGGAAAGAACT | 2670 |
| | AGTTCTTTCCTTCAAAT | 2671 |
| Haemophilia B | GGCGGCAGTTGCAAGGATGACATTAATTCCTATGAATGTTGG | 2672 |
| Gly79Arg | TGTCCCTTTGGATTTGAAGGAAAGAACTGTGAATTAGGTAAGT | |
| aGGA-AGA | AACTATTTTTTGAATACTCATGGTTCAAAGTTTCCC | |
| aGGA-AGA | GGGAAACTTTGAACCATGAGTATTCAAAAAATAGTTACTTACC | 2673 |
| | TAATTCACAGTTCTTTCCTTCAAATCCAAAGGGACACCAACAT | |
| | TCATAGGAATTAATGTCATCCTTGCAACTGCCGCC | |
| | GATTTGAAGGAAAGAAC | 2674 |
| | GTTCTTTCCTTCAAATC | 2675 |
| Haemophilia B | GCGGCAGTTGCAAGGATGACATTAATTCCTATGAATGTTGGT | 2676 |
| Gly79Glu | GTCCCTTTGGATTTGAAGGAAAGAACTGTGAATTAGGTAAGTA | |
| GGA-GAA | ACTATTTTTTGAATACTCATGGTTCAAAGTTTCCCT | |
| | AGGGAAACTTTGAACCATGAGTATTCAAAAAATAGTTACTTAC | 2677 |
| | CTAATTCACAGTTCTTTCCTTCAAATCCAAAGGGACACCAACA | |
| | TTCATAGGAATTAATGTCATCCTTGCAACTGCCGC | |
| | ATTTGAAGGAAAGAACT | 2678 |
| | AGTTCTTTCCTTCAAAT | 2679 |
| Haemophilia B | TTAGAAATGCATGTTAAATGATGCTGTTACTGTCTATTTTGCTT | 2680 |
| Cys88Ser | CTTTTAGATGTAACATGTAACATTAAGAATGGCAGATGCGAGC | |
| TGT-TCT | AGTTTTGTAAAAATAGTGCTGATAACAAGGTGGT | |
| | ACCACCTTGTTATCAGCACTATTTTTACAAAACTGCTCGCATC | 2681 |
| | TGCCATTCTTAATGTTACATGTTACATCTAAAAGAAGCAAAATA | |
| | GACAGTAACAGCATCATTTAACATGCATTTCTAA | |
| | TGTAACATGTAACATTA | 2682 |
| | TAATGTTACATGTTACA | 2683 |
| Haemophilia B | TTAGAAATGCATGTTAAATGATGCTGTTACTGTCTATTTTGCTT | 2684 |
| Cys88Phe | CTTTTAGATGTAACATGTAACATTAAGAATGGCAGATGCGAGC | |
| TGT-TTT | AGTTTTGTAAAAATAGTGCTGATAACAAGGTGGT | |
| | ACCACCTTGTTATCAGCACTATTTTTACAAAACTGCTCGCATC | 2685 |
| | TGCCATTCTTAATGTTACATGTTACATCTAAAAGAAGCAAAATA | |
| | GACAGTAACAGCATCATTTAACATGCATTTCTAA | |
| | TGTAACATGTAACATTA | 2686 |
| | TAATGTTACATGTTACA | 2687 |
| Haemophilia B | TTTAGAAATGCATGTTAAATGATGCTGTTACTGTCTATTTTGCT | 2688 |
| Cys88Arg | TCTTTTAGATGTAACATGTAACATTAAGAATGGCAGATGCGAG | |
| aTGT-CGT | CAGTTTTGTAAAAATAGTGCTGATAACAAGGTGG | |
| | CCACCTTGTTATCAGCACTATTTTTACAAAACTGCTCGCATCT | 2689 |
| | GCCATTCTTAATGTTACATGTTACATCTAAAAGAAGCAAAATA | |
| | GACAGTAACAGCATCATTTAACATGCATTTCTAAA | |
| | ATGTAACATGTAACATT | 2690 |
| | AATGTTACATGTTACAT | 2691 |
| Haemophilia B | TTAGAAATGCATGTTAAATGATGCTGTTACTGTCTATTTTGCTT | 2692 |
| Cys88Tyr | CTTTTAGATGTAACATGTAACATTAAGAATGGCAGATGCGAGC | |
| TGT-TAT | AGTTTTGTAAAAATAGTGCTGATAACAAGGTGGT | |
| | ACCACCTTGTTATCAGCACTATTTTTACAAAACTGCTCGCATC | 2693 |
| | TGCCATTCTTAATGTTACATGTTACATCTAAAAGAAGCAAAATA | |
| | GACAGTAACAGCATCATTTAACATGCATTTCTAA | |
| | TGTAACATGTAACATTA | 2694 |
| | TAATGTTACATGTTACA | 2695 |
| Haemophilia B | ATGCATGTTAAATGATGCTGTTACTGTCTATTTTGCTTCTTTTA | 2696 |
| Ile90Thr | GATGTAACATGTAACATTAAGAATGGCAGATGCGAGCAGTTTT | |
| ATT-ACT | GTAAAAATAGTGCTGATAACAAGGTGGTTTGCTC | |
| | GAGCAAACCACCTTGTTATCAGCACTATTTTTACAAAACTGCT | 2697 |
| | CGCATCTGCCATTCTTAATGTTACATGTTACATCTAAAAGAAG | |
| | CAAAATAGACAGTAACAGCATCATTTAACATGCAT | |
| | ATGTAACATTAAGAATG | 2698 |
| | CATTCTTAATGTTACAT | 2699 |
| Haemophilia B | TGTTAAATGATGCTGTTACTGTCTATTTTGCTTCTTTTAGATGT | 2700 |
| Asn92His | AACATGTAACATTAAGAATGGCAGATGCGAGCAGTTTTGTAAA | |
| gAAT-CAT | AATAGTGCTGATAACAAGGTGGTTTGCTCCTGTA | |
| | TACAGGAGCAAACCACCTTGTTATCAGCACTATTTTTACAAAA | 2701 |
| | CTGCTCGCATCTGCCATTCTTAATGTTACATGTTACATCTAAA | |
| | AGAAGCAAAATAGACAGTAACAGCATCATTTAACA | |
| | ACATTAAGAATGGCAGA | 2702 |
| | TCTGCCATTCTTAATGT | 2703 |
| Haemophilia B | TTAAATGATGCTGTTACTGTCTATTTTGCTTCTTTTAGATGTAA | 2704 |
| Asn92Lys | CATGTAACATTAAGAATGGCAGATGCGAGCAGTTTTGTAAAAA | |
| AATg-AAA | TAGTGCTGATAACAAGGTGGTTTGCTCCTGTACT | |
| | AGTACAGGAGCAAACCACCTTGTTATCAGCACTATTTTTACAA | 2705 |
| | AACTGCTCGCATCTGCCATTCTTAATGTTACATGTTACATCTA | |
| | AAAGAAGCAAAATAGACAGTAACAGCATCATTTAA | |
| | ATTAAGAATGGCAGATG | 2706 |
| | CATCTGCCATTCTTAAT | 2707 |
| Haemophilia B | AAATGATGCTGTTACTGTCTATTTTGCTTCTTTTAGATGTAACA | 2708 |
| Gly93Asp | TGTAACATTAAGAATGGCAGATGCGAGCAGTTTTGTAAAAATA | |
| GGC-GAC | GTGCTGATAACAAGGTGGTTTGCTCCTGTACTGA | |
| | TCAGTACAGGAGCAAACCACCTTGTTATCAGCACTATTTTTAC | 2709 |
| | AAAACTGCTCGCATCTGCCATTCTTAATGTTACATGTTACATC | |
| | TAAAAGAAGCAAAATAGACAGTAACAGCATCATTT | |
| | TAAGAATGGCAGATGCG | 2710 |
| | CGCATCTGCCATTCTTA | 2711 |
| Haemophilia B | TAAATGATGCTGTTACTGTCTATTTTGCTTCTTTTAGATGTAAC | 2712 |
| Gly93Ser | ATGTAACATTAAGAATGGCAGATGCGAGCAGTTTTGTAAAAAT | |
| tGGC-AGC | AGTGCTGATAACAAGGTGGTTTGCTCCTGTACTG | |
| | CAGTACAGGAGCAAACCACCTTGTTATCAGCACTATTTTTACA | 2713 |
| | AAACTGCTCGCATCTGCCATTCTTAATGTTACATGTTACATCT | |
| | AAAAGAAGCAAAATAGACAGTAACAGCATCATTTA | |
| | TTAAGAATGGCAGATGC | 2714 |
| | GCATCTGCCATTCTTAA | 2715 |
| Haemophilia B | GATGCTGTTACTGTCTATTTTGCTTCTTTTAGATGTAACATGTA | 2716 |
| Arg94Ser | ACATTAAGAATGGCAGATGCGAGCAGTTTTGTAAAAATAGTG | |
| AGAt-AGT | CTGATAACAAGGTGGTTTGCTCCTGTACTGAGGGA | |
| | TCCCTCAGTACAGGAGCAAACCACCTTGTTATCAGCACTATTT | 2717 |
| | TTACAAAACTGCTCGCATCTGCCATTCTTAATGTTACATGTTA | |
| | CATCTAAAAGAAGCAAAATAGACAGTAACAGCATC | |
| | AATGGCAGATGCGAGCA | 2118 |
| | TGCTCGCATCTGCCATT | 2719 |
| Haemophilia B | TGCTGTTACTGTCTATTTTGCTTCTTTTAGATGTAACATGTAAC | 2720 |
| Cys95Tyr | ATTAAGAATGGCAGATGCGAGCAGTTTTGTAAAAATAGTGCT | |
| TGC-TAC | GATAACAAGGTGGTTTGCTCCTGTACTGAGGGATA | |
| | TATCCCTCAGTACAGGAGCAAACCACCTTGTTATCAGCACTAT | 2721 |
| | TTTTACAAAACTGCTCGCATCTGCCATTCTTAATGTTACATGTT | |
| | ACATCTAAAAGAAGCAAAATAGACAGTAACAGCA | |
| | TGGCAGATGCGAGCAGT | 2722 |
| | ACTGCTCGCATCTGCCA | 2723 |
| Haemophilia B | GCTGTTACTGTCTATTTTGCTTCTTTTAGATGTAACATGTAACA | 2724 |
| Cys95Trp | TTAAGAATGGCAGATGCGAGCAGTTTTGTAAAAATAGTGCTG | |
| TGCg-TGG | ATAACAAGGTGGTTTGCTCCTGTACTGAGGGATAT | |
| | ATATCCCTCAGTACAGGAGCAAACCACCTTGTTATCAGCACTA | 2725 |
| | TTTTTACAAAACTGCTCGCATCTGCCATTCTTAATGTTACATGT | |
| | TACATCTAAAAGAAGCAAAATAGACAGTAACAGC | |
| | GGCAGATGCGAGCAGTT | 2726 |
| | AACTGCTCGCATCTGCC | 2727 |
| Haemophilia B | GCTGTTACTGTCTATTTTGCTTCTTTTAGATGTAACATGTAACA | 2728 |
| Cys95Term | TTAAGAATGGCAGATGCGAGCAGTTTTGTAAAAATAGTGCTG | |
| TGCg-TGA | ATAACAAGGTGGTTTGCTCCTGTACTGAGGGATAT | |
| | ATATCCCTCAGTACAGGAGCAAACCACCTTGTTATCAGCACTA | 2729 |
| | TTTTTACAAAACTGCTCGCATCTGCCATTCTTAATGTTACATGT | |
| | TACATCTAAAAGAAGCAAAATAGACAGTAACAGC | |
| | GGCAGATGCGAGCAGTT | 2730 |
| | AACTGCTCGCATCTGCC | 2731 |
| Haemophilia B | TACTGTCTATTTTGCTTCTTTTAGATGTAACATGTAACATTAAG | 2732 |
| Gln97Pro | AATGGCAGATGCGAGCAGTTTTGTAAAAATAGTGCTGATAAC | |
| CAG-CCG | AAGGTGGTTTGCTCCTGTACTGAGGGATATCGACT | |
| | AGTCGATATCCCTCAGTACAGGAGCAAACCACCTTGTTATCA | 2733 |
| | GCACTATTTTTACAAAACTGCTCGCATCTGCCATTCTTAATGTT | |
| | ACATGTTACATCTAAAAGAAGCAAAATAGACAGTA | |
| | ATGCGAGCAGTTTTGTA | 2734 |
| | TACAAAACTGCTCGCAT | 2735 |
| Haemophilia B | TTACTGTCTATTTTGCTTCTTTTAGATGTAACATGTAACATTAA | 2736 |
| Gln97Glu | GAATGGCAGATGCGAGCAGTTTTGTAAAAATAGTGCTGATAA | |
| gCAG-GAG | CAAGGTGGTTTGCTCCTGTACTGAGGGATATCGAC | |
| | GTCGATATCCCTCAGTACAGGAGCAAACCACCTTGTTATCAG | 2737 |
| | CACTATTTTTACAAAACTGCTCGCATCTGCCATTCTTAATGTTA | |
| | CATGTTACATCTAAAAGAAGCAAAATAGACAGTAA | |
| | GATGCGAGCAGTTTTGT | 2738 |
| | ACAAAACTGCTCGCATC | 2739 |
| Haemophilia B | TCTATTTTGCTTCTTTTAGATGTAACATGTAACATTAAGAATGG | 2740 |
| Cys99Arg | CAGATGCGAGCAGTTTTGTAAAAATAGTGCTGATAACAAGGT | |
| tTGT-CGT | GGTTTGCTCCTGTACTGAGGGATATCGACTTGCAG | |
| | CTGCAAGTCGATATCCCTCAGTACAGGAGCAAACCACCTTGT | 2741 |
| | TATCAGCACTATTTTTACAAAACTGCTCGCATCTGCCATTCTTA | |
| | ATGTTACATGTTACATCTAAAAGAAGCAAAATAGA | |
| | AGCAGTTTTGTAAAAAT | 2742 |
| | ATTTTTACAAAACTGCT | 2743 |
| Haemophilia B | CTATTTTGCTTCTTTTAGATGTAACATGTAACATTAAGAATGGC | 2744 |
| Cys99Tyr | AGATGCGAGCAGTTTTGTAAAAATAGTGCTGATAACAAGGTG | |
| TGT-TAT | GTTTGCTCCTGTACTGAGGGATATCGACTTGCAGA | |
| | TCTGCAAGTCGATATCCCTCAGTACAGGAGCAAACCACCTTG | 2745 |
| | TTATCAGCACTATTTTTACAAAACTGCTCGCATCTGCCATTCTT | |
| | AATGTTACATGTTACATCTAAAAGAAGCAAAATAG | |
| | GCAGTTTTGTAAAAATA | 2746 |
| | TATTTTTACAAAACTGC | 2747 |
| Warfarin sensitivity | TTTTTTGCTAAAACTAAAGAATTATTCTTTTACATTTCAGTTTTT | 2748 |
| Ala(−10)Thr | CTTGATCATGAAAACGCCAACAAAATTCTGAATCGGCCAAAGA | |
| cGCC-ACC | GGTATAATTCAGGTAAATTGGAAGAGTTTGTTC | |
| | GAACAAACTCTTCCAATTTACCTGAATTATACCTCTTTGGCCG | 2749 |
| | ATTCAGAATTTTGTTGGCGTTTTCATGATCAAGAAAAACTGAA | |
| | ATGTAAAAGAATAATTCTTTAGTTTTAGCAAAAAA | |
| | ATGAAAACGCCAACAAA | 2750 |
| | TTTGTTGGCGTTTTCAT | 2751 |
| Warfarin sensitivity | TTTTTGCTAAAACTAAAGAATTATTCTTTTACATTTCAGTTTTTC | 2752 |
| Ala(−10)Val | TTGATCATGAAAACGCCAACAAAATTCTGAATCGGCCAAAGA | |
| GCC-GTC | GGTATAATTCAGGTAAATTGGAAGAGTTTGTTCA | |
| | TGAACAAACTCTTCCAATTTACCTGAATTATACCTCTTTGGCC | 2753 |
| | GATTCAGAATTTTGTTGGCGTTTTCATGATCAAGAAAAACTGA | |
| | AATGTAAAAGAATAATTCTTTAGTTTTAGCAAAAA | |
| | TGAAAACGCCAACAAAA | 2754 |
| | TTTTGTTGGCGTTTTCA | 2755 |
| Haemophilia B | TGCAGCGCGTGAACATGATCATGGCAGAATCACCAGGCCTCA | 2756 |
| Gly(−26)Val | TCACCATCTGCCTTTTAGGATATCTACTCAGTGCTGAATGTAC | |
| GGA-GTA | AGGTTTGTTTCCTTTTTTAAAATACATTGAGTATGC | |
| | GCATACTCAATGTATTTTAAAAAAGGAAACAAACCTGTACATT | 2757 |
| | CAGCACTGAGTAGATATCCTAAAAGGCAGATGGTGATGAGGC | |
| | CTGGTGATTCTGCCATGATCATGTTCACGCGCTGCA | |
| | CCTTTTAGGATATCTAC | 2758 |
| | GTAGATATCCTAAAAGG | 2759 |
| Haemophilia B | TTATGCAGCGCGTGAACATGATCATGGCAGAATCACCAGGCC | 2760 |
| Leu(−27)Term | TCATCACCATCTGCCTTTTAGGATATCTACTCAGTGCTGAATG | |
| TTA-TAA | TACAGGTTTGTTTCCTTTTTTAAAATACATTGAGTA | |
| TTA-TAA | TACTCAATGTATTTTAAAAAAGGAAACAAACCTGTACATTCAG | 2761 |
| | CACTGAGTAGATATCCTAAAAGGCAGATGGTGATGAGGCCTG | |
| | GTGATTCTGCCATGATCATGTTCACGCGCTGCATAA | |
| | CTGCCTTTTAGGATATC | 2762 |
| | GATATCCTAAAAGGCAG | 2763 |
| Haemophilia B | TAGCAAAGGTTATGCAGCGCGTGAACATGATCATGGCAGAAT | 2764 |
| Ile(−30)Asn | CACCAGGCCTCATCACCATCTGCCTTTTAGGATATCTACTCAG | |
| ATC-AAC | TGCTGAATGTACAGGTTTGTTTCCTTTTTTAAAATA | |
| | TATTTTAAAAAAGGAAACAAACCTGTACATTCAGCACTGAGTA | 2765 |
| | GATATCCTAAAAGGCAGATGGTGATGAGGCCTGGTGATTCTG | |
| | CCATGATCATGTTCACGCGCTGCATAACCTTTGCTA | |
| | CATCACCATCTGCCTTT | 2766 |
| | AAAGGCAGATGGTGATG | 2767 |
| Haemophilia B | ACTAATCGACCTTACCACTTTCACAATCTGCTAGCAAAGGTTA | 2768 |
| Ile(−40)Phe | TGCAGCGCGTGAACATGATCATGGCAGAATCACCAGGCCTCA | |
| gATC-TTC | TCACCATCTGCCTTTTAGGATATCTACTCAGTGCTG | |
| | CAGCACTGAGTAGATATCCTAAAAGGCAGATGGTGATGAGGC | 2769 |
| | CTGGTGATTCTGCCATGATCATGTTCACGCGCTGCATAACCTT | |
| | TGCTAGCAGATTGTGAAAGTGGTAAGGTCGATTAGT | |
| | TGAACATGATCATGGCA | 2770 |
| | TGCCATGATCATGTTCA | 2771 |
| Haemophilia B | ACTTTGGTACAACTAATCGACCTTACCACTTTCACAATCTGCT | 2772 |
| Arg(−44)His | AGCAAAGGTTATGCAGCGCGTGAACATGATCATGGCAGAATC | |
| CGC-CAC | ACCAGGCCTCATCACCATCTGCCTTTTAGGATATCT | |
| | AGATATCCTAAAAGGCAGATGGTGATGAGGCCTGGTGATTCT | 2773 |
| | GCCATGATCATGTTCACGCGCTGCATAACCTTTGCTAGCAGA | |
| | TTGTGAAAGTGGTAAGGTCGATTAGTTGTACCAAAGT | |
| | TATGCAGCGCGTGAACA | 2774 |
| | TGTTCACGCGCTGCATA | 2775 |

### EXAMPLE 16

### Alpha Thalassemia Hemoglobin Alpha Locus 1

The thalassemia syndromes are a heterogeneous group of inherited anemias characterized by defects in the synthesis of one or more globin chain subunits. For example, beta-thalassemia discussed in Example 6, is caused by a decrease in beta-chain production relative to alpha-chain production; the converse is the case for alpha-thalassemia. The attached table discloses the correcting oligonucleotide base sequences for the hemoglobin alpha locus 1 oligonucleotides of the invention.

TABLE 18
HBA1 Mutations and Genome-Correcting Oligos

| Clinical Phenotype & | | SEQ ID |
|---|---|---|
| Mutation | Correcting Oligos | NO: |
| Thalassaemia alpha | CCCTGGCGCGCTCGCGGCCCGGCACTCTTCTGGTCCCCACA | 2776 |
| Met(−1)Val | GACTCAGAGAGAACCCACCATGGTGCTGTCTCCTGCCGACAA | |
| cATG-GTG | GACCAACGTCAAGGCCGCCTGGGGTAAGGTCGGCGCGC | |
| | GCGCGCCGACCTTACCCCAGGCGGCCTTGACGTTGGTCTTG | 2777 |
| | TCGGCAGGAGACAGCACCATGGTGGGTTCTCTCTGAGTCTGT | |
| | GGGGACCAGAAGAGTGCCGGGCCGCGAGCGCGCCAGGG | |
| | AACCCACCATGGTGCTG | 2778 |
| | CAGCACCATGGTGGGTT | 2779 |
| Haemoglobin variant | CACAGACTCAGAGAGAACCCACCATGGTGCTGTCTCCTGCCG | 2780 |
| Ala12Asp | ACAAGACCAACGTCAAGGCCGCCTGGGGTAAGGTCGGCGCG | |
| GCC-GAC | CACGCTGGCGAGTATGGTGCGGAGGCCCTGGAGAGGTG | |
| | CACCTCTCCAGGGCCTCCGCACCATACTCGCCAGCGTGCGC | 2781 |
| | GCCGACCTTACCCCAGGCGGCCTTGACGTTGGTCTTGTCGG | |
| | CAGGAGACAGCACCATGGTGGGTTCTCTCTGAGTCTGTG | |
| | CGTCAAGGCCGCCTGGG | 2782 |
| | CCCAGGCGGCCTTGACG | 2783 |
| Haemoglobin variant | AGAGAGAACCCACCATGGTGCTGTCTCCTGCCGACAAGACCA | 2784 |
| Gly15Asp | ACGTCAAGGCCGCCTGGGGTAAGGTCGGCGCGCACGCTGG | |
| GGT-GAT | CGAGTATGGTGCGGAGGCCCTGGAGAGGTGAGGCTCCCT | |
| | AGGGAGCCTCACCTCTCCAGGGCCTCCGCACCATACTCGCC | 2785 |
| | AGCGTGCGCGCCGACCTTACCCCAGGCGGCCTTGACGTTGG | |
| | TCTTGTCGGCAGGAGACAGCACCATGGTGGGTTCTCTCT | |
| | CGCCTGGGGTAAGGTCG | 2786 |
| | CGACCTTACCCCAGGCG | 2787 |
| Haemoglobin variant | CTGCCGACAAGACCAACGTCAAGGCCGCCTGGGGTAAGGTC | 2788 |
| Tyr24Cys | GGCGCGCACGCTGGCGAGTATGGTGCGGAGGCCCTGGAGA | |
| TAT-TGT | GGTGAGGCTCCCTCCCCTGCTCCGACCCGGGCTCCTCGCC | |
| | GGCGAGGAGCCCGGGTCGGAGCAGGGGAGGGAGCCTCACC | 2789 |
| | TCTCCAGGGCCTCCGCACCATACTCGCCAGCGTGCGCGCCG | |
| | ACCTTACCCCAGGCGGCCTTGACGTTGGTCTTGTCGGCAG | |
| | TGGCGAGTATGGTGCGG | 2790 |
| | CCGCACCATACTCGCCA | 2791 |
| Haemoglobin variant | GACCAACGTCAAGGCCGCCTGGGGTAAGGTCGGCGCGCAC | 2792 |
| Glu27Asp | GCTGGCGAGTATGGTGCGGAGGCCCTGGAGAGGTGAGGCT | |
| GAGg-GAT | CCCTCCCCTGCTCCGACCCGGGCTCCTCGCCCGCCCGGACC | |
| | GGTCCGGGCGGGCGAGGAGCCCGGGTCGGAGCAGGGGAG | 2793 |
| | GGAGCCTCACCTCTCCAGGGCCTCCGCACCATACTCGCCAG | |
| | CGTGCGCGCCGACCTTACCCCAGGCGGCCTTGACGTTGGTC | |
| | GGTGCGGAGGCCCTGGA | 2794 |
| | TCCAGGGCCTCCGCACC | 2795 |
| Haemoglobin variant | GAGCCACGGCTCTGCCCAGGTTAAGGGCCACGGCAAGAAGG | 2796 |
| Asn68Lys | TGGCCGACGCGCTGACCAACGCCGTGGCGCACGTGGACGA | |
| AACg-AAG | CATGCCCAACGCGCTGTCCGCCCTGAGCGACCTGCACGCG | |
| | CGCGTGCAGGTCGCTCAGGGCGGACAGCGCGTTGGGCATGT | 2797 |
| | CGTCCACGTGCGCCACGGCGTTGGTCAGCGCGTCGGCCACC | |
| | TTCTTGCCGTGGCCCTTAACCTGGGCAGAGCCGTGGCTC | |
| | CTGACCAACGCCGTGGC | 2798 |
| | GCCACGGCGTTGGTCAG | 2799 |
| Haemoglobin variant | AGGTTAAGGGCCACGGCAAGAAGGTGGCCGACGCGCTGACC | 2800 |
| Asp74Gly | AACGCCGTGGCGCACGTGGACGACATGCCCAACGCGCTGTC | |
| GAC-GGC | CGCCCTGAGCGACCTGCACGCGCACAAGCTTCGGGTGGA | |
| | TCCACCCGAAGCTTGTGCGCGTGCAGGTCGCTCAGGGCGGA | 2801 |
| | CAGCGCGTTGGGCATGTCGTCCACGTGCGCCACGGCGTTGG | |
| | TCAGCGCGTCGGCCACCTTCTTGCCGTGGCCCTTAACCT | |
| | GCACGTGGACGACATGC | 2802 |
| | GCATGTCGTCCACGTGC | 2803 |
| Haemoglobin variant | CAGGTTAAGGGCCACGGCAAGAAGGTGGCCGACGCGCTGAC | 2804 |
| Asp74His | CAACGCCGTGGCGCACGTGGACGACATGCCCAACGCGCTGT | |
| gGAC-CAC | CCGCCCTGAGCGACCTGCACGCGCACAAGCTTCGGGTGG | |
| | CCACCCGAAGCTTGTGCGCGTGCAGGTCGCTCAGGGCGGAC | 2805 |
| | AGCGCGTTGGGCATGTCGTCCACGTGCGCCACGGCGTTGGT | |
| | CAGCGCGTCGGCCACCTTCTTGCCGTGGCCCTTAACCTG | |
| | CGCACGTGGACGACATG | 2806 |
| | CATGTCGTCCACGTGCG | 2807 |
| Haemoglobin variant | CACGGCAAGAAGGTGGCCGACGCGCTGACCAACGCCGTGG | 2808 |
| Asn78His | CGCACGTGGACGACATGCCCAACGCGCTGTCCGCCCTGAGC | |
| cAAC-CAC | GACCTGCACGCGCACAAGCTTCGGGTGGACCCGGTCAACT | |
| | AGTTGACCGGGTCCACCCGAAGCTTGTGCGCGTGCAGGTCG | 2809 |
| | CTCAGGGCGGACAGCGCGTTGGGCATGTCGTCCACGTGCGC | |
| | CACGGCGTTGGTCAGCGCGTCGGCCACCTTCTTGCCGTG | |
| | ACATGCCCAACGCGCTG | 2810 |
| | CAGCGCGTTGGGCATGT | 2811 |
| Haemoglobin variant | ACCAACGCCGTGGCGCACGTGGACGACATGCCCAACGCGCT | 2812 |
| His87Tyr | GTCCGCCCTGAGCGACCTGCACGCGCACAAGCTTCGGGTGG | |
| gCAC-TAC | ACCCGGTCAACTTCAAGGTGAGCGGCGGGCCGGGAGCGA | |
| | TCGCTCCCGGCCCGCCGCTCACCTTGAAGTTGACCGGGTCC | 2813 |
| | ACCCGAAGCTTGTGCGCGTGCAGGTCGCTCAGGGCGGACAG | |
| | CGCGTTGGGCATGTCGTCCACGTGCGCCACGGCGTTGGT | |
| | GCGACCTGCACGCGCAC | 2814 |
| | GTGCGCGTGCAGGTCGC | 2815 |
| Haemoglobin variant | GGCGCACGTGGACGACATGCCCAACGCGCTGTCCGCCCTGA | 2816 |
| Lys90Asn | GCGACCTGCACGCGCACAAGCTTCGGGTGGACCCGGTCAAC | |
| AAGc-AAC | TTCAAGGTGAGCGGCGGGCCGGGAGCGATCTGGGTCGAG | |
| | CTCGACCCAGATCGCTCCCGGCCCGCCGCTCACCTTGAAGT | 2817 |
| | TGACCGGGTCCACCCGAAGCTTGTGCGCGTGCAGGTCGCTC | |
| | AGGGCGGACAGCGCGTTGGGCATGTCGTCCACGTGCGCC | |
| | GCGCACAAGCTTCGGGT | 2818 |
| | ACCCGAAGCTTGTGCGC | 2819 |
| Haemoglobin variant | TGGCGCACGTGGACGACATGCCCAACGCGCTGTCCGCCCTG | 2820 |
| Lys90Thr | AGCGACCTGCACGCGCACAAGCTTCGGGTGGACCCGGTCAA | |
| AAG-ACG | CTTCAAGGTGAGCGGCGGGCCGGGAGCGATCTGGGTCGA | |
| | TCGACCCAGATCGCTCCCGGCCCGCCGCTCACCTTGAAGTT | 2821 |
| | GACCGGGTCCACCCGAAGCATGTGCGCGTGCAGGTCGCTCA | |
| | GGGCGGACAGCGCGTTGGGCATGTCGTCCACGTGCGCCA | |
| | CGCGCACAAGCTTCGGG | 2822 |
| | CCCGAAGCATGTGCGCG | 2823 |
| Haemoglobin variant | ACGTGGACGACATGCCCAACGCGCTGTCCGCCCTGAGCGAC | 2824 |
| Arg92Gln | CTGCACGCGCACAAGCTTCGGGTGGACCCGGTCAACTTCAA | |
| CGG-CAG | GGTGAGCGGCGGGCCGGGAGCGATCTGGGTCGAGGGGCG | |
| | CGCCCCTCGACCCAGATCGCTCCCGGCCCGCCGCTCACCTT | 2825 |
| | GAAGTTGACCGGGTCCACCCGAAGCTTGTGCGCGTGCAGGT | |
| | CGCTCAGGGCGGACAGCGCGTTGGGCATGTCGTCCACGT | |
| | CAAGCTTCGGGTGGACC | 2826 |
| | GGTCCACCCGAAGCTTG | 2827 |
| Haemoglobin variant | ACGACATGCCCAACGCGCTGTCCGCCCTGAGCGACCTGCAC | 2828 |
| Asp94Gly | GCGCACAAGCTTCGGGTGGACCCGGTCAACTTCAAGGTGAG | |
| GAC-GGC | CGGCGGGCCGGGAGCGATCTGGGTCGAGGGGCGAGATGG | |
| | CCATCTCGCCCCTCGACCCAGATCGCTCCCGGCCCGCCGCT | 2829 |
| | CACCTTGAAGTTGACCGGGTCCACCCGAAGCTTGTGCGCGT | |
| | GCAGGTCGCTCAGGGCGGACAGCGCGTTGGGCATGTCGT | |
| | TCGGGTGGACCCGGTCA | 2830 |
| | TGACCGGGTCCACCCGA | 2831 |
| Haemoglobin variant | ACATGCCCAACGCGCTGTCCGCCCTGAGCGACCTGCACGCG | 2832 |
| Pro95Arg | CACAAGCTTCGGGTGGACCCGGTCAACTTCAAGGTGAGCGG | |
| CCG-CGG | CGGGCCGGGAGCGATCTGGGTCGAGGGGCGAGATGGCGC | |
| | GCGCCATCTCGCCCCTCGACCCAGATCGCTCCCGGCCCGCC | 2833 |
| | GCTCACCTTGAAGTTGACCGGGTCCACCCGAAGCTTGTGCG | |
| | CGTGCAGGTCGCTCAGGGCGGACAGCGCGTTGGGCATGT | |
| | GGTGGACCCGGTCAACT | 2834 |
| | AGTTGACCGGGGTCCACC | 2835 |
| Haemoglobin variant | CGGCGGCTGCGGGCCTGGGCCCTCGGCCCCACTGACCCTC | 2836 |
| Ser102Arg | TTCTCTGCACAGCTCCTAAGCCACTGCCTGCTGGTGACCCTG | |
| AGCc-AGA | GCCGCCCACCTCCCCGCCGAGTTCACCCCTGCGGTGCAC | |
| | GTGCACCGCAGGGGTGAACTCGGCGGGGAGGTGGGCGGCC | 2837 |
| | AGGGTCACCAGCAGGCAGTGGCTTAGGAGCTGTGCAGAGAA | |
| | GAGGGTCAGTGGGGCCGAGGGCCCAGGCCCGCAGCCGCCG | |
| | CTCCTAAGCCACTGCCT | 2838 |
| | AGGCAGTGGCTTAGGAG | 2839 |
| Haemoglobin variant | TTCTCTGCACAGCTCCTAAGCCACTGCCTGCTGGTGACCCTG | 2840 |
| Glu116Lys | GCCGCCCACCTCCCCGCCGAGTTCACCCCTGCGGTGCACGC | |
| cGAG-AAG | CTCCCTGGACAAGTTCCTGGCTTCTGTGAGCACCGTGC | |
| | GCACGGTGCTCACAGAAGCCAGGAACTTGTCCAGGGAGGCG | 2841 |
| | TGCACCGCAGGGGTGAACTCGGCGGGGAGGTGGGCGGCCA | |
| | GGGTCACCAGCAGGCAGTGGCTTAGGAGCTGTGCAGAGAA | |
| | TCCCCGCCGAGTTCACC | 2842 |
| | GGTGAACTCGGCGGGGA | 2843 |
| Haemoglobin variant | TCCTAAGCCACTGCCTGCTGGTGACCCTGGCCGCCCACCTC | 2844 |
| Ala120Glu | CCCGCCGAGTTCACCCCTGCGGTGCACGCCTCCCTGGACAA | |
| GCG-GAG | GTTCCTGGCTTCTGTGAGCACCGTGCTGACCTCCAAATA | |
| | TATTTGGAGGTCAGCACGGTGCTCACAGAAGCCAGGAACTTG | 2845 |
| | TCCAGGGAGGCGTGCACCGCAGGGGTGAACTCGGCGGGGA | |
| | GGTGGGCGGCCAGGGTCACCAGCAGGCAGTGGCTTAGGA | |
| | CACCCCTGCGGTGCACG | 2846 |
| | CGTGCACCGCAGGGGTG | 2847 |
| Thalassaemia alpha | TGGCCGCCCACCTCCCCGCCGAGTTCACCCCTGCGGTGCAC | 2848 |
| Leu129Pro | GCCTCCCTGGACAAGTTCCTGGCTTCTGTGAGCACCGTGCTG | |
| CTG-CCG | ACCTCCAAATACCGTTAAGCTGGAGCCTCGGTGGCCAT | |
| | ATGGCCACCGAGGCTCCAGCTTAACGGTATTTGGAGGTCAGC | 2849 |
| | ACGGTGCTCACAGAAGCCAGGAACTTGTCCAGGGAGGCGTG | |
| | CACCGCAGGGGTGAACTCGGCGGGGAGGTGGGCGGCCA | |
| | CAAGTTCCTGGCTTCTG | 2850 |
| | CAGAAGCCAGGAACTTG | 2851 |
| Haemoglobin variant | TGCACGCCTCCCTGGACAAGTTCCTGGCTTCTGTGAGCACCG | 2852 |
| Arg141Leu | TGCTGACCTCCAAATACCGTTAAGCTGGAGCCTCGGTGGCCA | |
| CGT-CTT | TGCTTCTTGCCCCTTGGGCCTCCCCCCAGCCCCTCCT | |
| | AGGAGGGGCTGGGGGGAGGCCCAAGGGGCAAGAAGCATGG | 2853 |
| | CCACCGAGGCTCCAGCTTAACGGTATTTGGAGGTCAGCACG | |
| | GTGCTCACAGAAGCCAGGAACTTGTCCAGGGAGGCGTGCA | |
| | CAAATACCGTTAAGCTG | 2854 |
| | CAGCTTAACGGTATTTG | 2855 |

### EXAMPLE 17

### Alpha-Thalassemia—Hemoglobin Alpha Locus 2

The attached table discloses the correcting oligonucleotide base sequences for the hemoglobin alpha locus 2 oligonucleotides of the invention.

TABLE 19
HBA2 Mutations and Genome-Correcting Oligos

| Clinical Phenotype & | | SEQ ID |
|---|---|---|
| Mutation | Correcting Oligos | NO: |
| Thalassaemia alpha | CCTGGCGCGCTCGCGGGCCGGCACTCTTCTGGTCCCCACAG | 2856 |
| Met(−1)Thr | ACTCAGAGAGAACCCACCATGGTGCTGTCTCCTGCCGACAAG | |
| ATG-ACG | ACCAACGTCAAGGCCGCCTGGGGTAAGGTCGGCGCGCA | |
| | TGCGCGCCGACCTTACCCCAGGCGGCCTTGACGTTGGTCTT | 2857 |
| | GTCGGCAGGAGACAGCACCATGGTGGGTTCTCTCTGAGTCT | |
| | GTGGGGACCAGAAGAGTGCCGGCCCGCGAGCGCGCCAGG | |
| | ACCCACCATGGTGCTGT | 2858 |
| | ACAGCACCATGGTGGGT | 2859 |
| Haemoglobin variant | CACAGACTCAGAGAGAACCCACCATGGTGCTGTCTCCTGCCG | 2860 |
| Ala12Asp | ACAAGACCAACGTCAAGGCCGCCTGGGGTAAGGTCGGCGCG | |
| GCC-GAC | CACGCTGGCGAGTATGGTGCGGAGGCCCTGGAGAGGTG | |
| | CACCTCTCCAGGGCCTCCGCACCATACTCGCCAGCGTGCGC | 2861 |
| | GCCGACCTTACCCCAGGCGGCCTTGACGTTGGTCTTGTCGG | |
| | CAGGAGACAGCACCATGGTGGGTTCTCTCTGAGTCTGTG | |
| | CGTCAAGGCCGCCTGGG | 2862 |
| | CCCAGGCGGCCTTGACG | 2863 |
| Haemoglobin variant | AGAGAACCCACCATGGTGCTGTCTCCTGCCGACAAGACCAAC | 2864 |
| Lys16Glu | GTCAAGGCCGCCTGGGGTAAGGTCGGCGCGCACGCTGGCG | |
| tAAG-GAG | AGTATGGTGCGGAGGCCCTGGAGAGGTGAGGCTCCCTCC | |
| | GGAGGGAGCCTCACCTCTCCAGGGCCTCCGCACCATACTCG | 2865 |
| | CCAGCGTGCGCGCCGACCTTACCCCAGGCGGCCTTGACGTT | |
| | GGTCTTGTCGGCAGGAGACAGCACCATGGTGGGTTCTCT | |
| | CCTGGGGTAAGGTCGGC | 2866 |
| | GCCGACCTTACCCCAGG | 2867 |
| Haemoglobin variant | GGTGCTGTCTCCTGCCGACAAGACCAACGTCAAGGCCGCCT | 2868 |
| His20Gln | GGGGTAAGGTCGGCGCGCACGCTGGCGAGTATGGTGCGGA | |
| CACg-CAA | GGCCCTGGAGAGGTGAGGCTCCCTCCCCTGCTCCGACCCG | |
| | CGGGTCGGAGCAGGGGAGGGAGCCTCACCTCTCCAGGGCC | 2869 |
| | TCCGCACCATACTCGCCAGCGTGCGCGCCGACCTTACCCCA | |
| | GGCGGCCTTGACGTTGGTCTTGTCGGCAGGAGACAGCACC | |
| | GGCGCGCACGCTGGCGA | 2870 |
| | TCGCCAGCGTGCGCGCC | 2871 |
| Haemoglobin variant | GACCAACGTCAAGGCCGCCTGGGGTAAGGTCGGCGCGCAC | 2872 |
| Glu27Asp | GCTGGCGAGTATGGTGCGGAGGCCCTGGAGAGGTGAGGCT | |
| GAGg-GAC | CCCTCCCCTGCTCCGACCCGGGCTCCTCGCCCGCCCGGACC | |
| | GGTCCGGGCGGGCGAGGAGCCCGGGTCGGAGCAGGGGAG | 2873 |
| | GGAGCCTCACCTCTCCAGGGCCTCCGCACCATACTCGCCAG | |
| | CGTGCGCGCCGACCTTACCCCAGGCGGCCTTGACGTTGGTC | |
| | GGTGCGGAGGCCCTGGA | 2874 |
| | TCCAGGGCCTCCGCACC | 2875 |
| Thalassaemia alpha | ACGTCAAGGCCGCCTGGGGTAAGGTCGGCGCGCACGCTGG | 2876 |
| Leu29Pro | CGAGTATGGTGCGGAGGCCCTGGAGAGGTGAGGCTCCCTCC | |
| CTG-CCG | CCTGCTCCGACCCGGGCTCCTCGCCCGCCCGGACCCACAG | |
| | CTGTGGGTCCGGGCGGGCGAGGAGCCCGGGTCGGAGCAGG | 2877 |
| | GGAGGGAGCCTCACCTCTCCAGGGCCTCCGCACCATACTCG | |
| | CCAGCGTGCGCGCCGACCTTACCCCAGGCGGCCTTGACGT | |
| | GGAGGCCCTGGAGAGGT | 2878 |
| | ACCTCTCCAGGGCCTCC | 2879 |
| Haemoglobin variant | GCTTCTCCCCGCAGGATGTTCCTGTCCTTCCCCACCACCAAG | 2880 |
| Asp47His | ACCTACTTCCCGCACTTCGACCTGAGCCACGGCTCTGCCCAG | |
| cGAC-CAC | GTTAAGGGCCACGGCAAGAAGGTGGCCGACGCGCTGA | |
| | TCAGCGCGTCGGCCACCTTCTTGCCGTGGCCCTTAACCTGG | 2881 |
| | GCAGAGCCGTGGCTCAGGTCGAAGTGCGGGAAGTAGGTCTT | |
| | GGTGGTGGGGAAGGACAGGAACATCCTGCGGGGAGAAGC | |
| | CGCACTTCGACCTGAGC | 2882 |
| | GCTCAGGTCGAAGTGCG | 2883 |
| Haemoglobin variant | CTCCCCGCAGGATGTTCCTGTCCTTCCCCACCACCAAGACCT | 2884 |
| Leu48Arg | ACTTCCCGCACTTCGACCTGAGCCACGGCTCTGCCCAGGTTA | |
| CTG-CGG | AGGGCCACGGCAAGAAGGTGGCCGACGCGCTGACCAA | |
| | TTGGTCAGCGCGTCGGCCACCTTCTTGCCGTGGCCCTTAACC | 2885 |
| | TGGGCAGAGCCGTGGCTCAGGTCGAAGTGCGGGAAGTAGGT | |
| | CTTGGTGGTGGGGAAGGACAGGAACATCCTGCGGGGAG | |
| | CTTCGACCTGAGCCACG | 2886 |
| | CGTGGCTCAGGTCGAAG | 2887 |
| Haemoglobin variant | CTGTCCTTCCCCACCACCAAGACCTACTTCCCGCACTTCGAC | 2888 |
| Gln54Glu | CTGAGCCACGGCTCTGCCCAGGTTAAGGGCCACGGCAAGAA | |
| cCAG-GAG | GGTGGCCGACGCGCTGACCAACGCCGTGGCGCACGTGG | |
| | CCACGTGCGCCACGGCGTTGGTCAGCGCGTCGGCCACCTTC | 2889 |
| | TTGCCGTGGCCCTTAACCTGGGCAGAGCCGTGGCTCAGGTC | |
| | GAAGTGCGGGAAGTAGGTCTTGGTGGTGGGGAAGGACAG | |
| | GCTCTGCCCAGGTTAAG | 2890 |
| | CTTAACCTGGGCAGAGC | 2891 |
| Haemoglobin variant | CCAAGACCTACTTCCCGCACTTCGACCTGAGCCACGGCTCTG | 2892 |
| Gly59Asp | CCCAGGTTAAGGGCCACGGCAAGAAGGTGGCCGACGCGCTG | |
| GGC-GAC | ACCAACGCCGTGGCGCACGTGGACGACATGCCCAACGC | |
| | GCGTTGGGCATGTCGTCCACGTGCGCCACGGCGTTGGTCAG | 2893 |
| | CGCGTCGGCCACCTTCTTGCCGTGGCCCTTAACCTGGGCAG | |
| | AGCCGTGGCTCAGGTCGAAGTGCGGGAAGTAGGTCTTGG | |
| | GGGCCACGGCAAGAAGG | 2894 |
| | CCTTCTTGCCGTGGCCC | 2895 |
| Haemoglobin variant | GAGCCACGGCTCTGCCCAGGTTAAGGGCCACGGCAAGAAGG | 2896 |
| Asn68Lys | TGGCCGACGCGCTGACCAACGCCGTGGCGCACGTGGACGA | |
| AACg-AAG | CATGCCCAACGCGCTGTCCGCCCTGAGCGACCTGCACGCG | |
| | CGCGTGCAGGTCGCTCAGGGCGGACAGCGCGTTGGGCATGT | 2897 |
| | CGTCCACGTGCGCCACGGCGTTGGTCAGCGCGTCGGCCACC | |
| | TTCTTGCCGTGGCCCTTAACCTGGGCAGAGCCGTGGCTC | |
| | CTGACCAACGCCGTGGC | 2898 |
| | GCCACGGCGTTGGTCAG | 2899 |
| Haemoglobin variant | GAGCCACGGCTCTGCCCAGGTTAAGGGCCACGGCAAGAAGG | 2900 |
| Asn68Lys | TGGCCGACGCGCTGACCAACGCCGTGGCGCACGTGGACGA | |
| AACg-AAA | CATGCCCAACGCGCTGTCCGCCCTGAGCGACCTGCACGCG | |
| | CGCGTGCAGGTCGCTCAGGGCGGACAGCGCGTTGGGCATGT | 2901 |
| | CGTCCACGTGCGCCACGGCGTTGGTCAGCGCGTCGGCCACC | |
| | TTCTTGCCGTGGCCCTTAACCTGGGCAGAGCCGTGGCTC | |
| | CTGACCAACGCCGTGGC | 2902 |
| | GCCACGGCGTTGGTCAG | 2903 |
| Haemoglobin variant | CGGCAAGAAGGTGGCCGACGCGCTGACCAACGCCGTGGCG | 2904 |
| Asn78Lys | CACGTGGACGACATGCCCAACGCGCTGTCCGCCCTGAGCGA | |
| AACg-AAA | CCTGCACGCGCACAAGCTTCGGGTGGACCCGGTCAACTTC | |
| | GAAGTTGACCGGGTCCACCCGAAGCTTGTGCGCGTGCAGGT | 2905 |
| | CGCTCAGGGCGGACAGCGCGTTGGGCATGTCGTCCACGTGC | |
| | GCCACGGCGTTGGTCAGCGCGTCGGCCACCTTCTTGCCG | |
| | ATGCCCAACGCGCTGTC | 2906 |
| | GACAGCGCGTTGGGCAT | 2907 |
| Haemoglobin variant | CGCTGACCAACGCCGTGGCGCACGTGGACGACATGCCCAAC | 2908 |
| Asp85Val | GCGCTGTCCGCCCTGAGCGACCTGCACGCGCACAAGCTTCG | |
| GAC-GTC | GGTGGACCCGGTCAACTTCAAGGTGAGCGGCGGGCCGGG | |
| | CCCGGCCCGCCGCTCACCTTGAAGTTGACCGGGTCCACCCG | 2909 |
| | AAGCTTGTGCGCGTGCAGGTCGCTCAGGGCGGACAGCGCGT | |
| | TGGGCATGTCGTCCACGTGCGCCACGGCGTTGGTCAGCG | |
| | CCTGAGCGACCTGCACG | 2910 |
| | CGTGCAGGTCGCTCAGG | 2911 |
| Haemoglobin variant | GGCGCACGTGGACGACATGCCCAACGCGCTGTCCGCCCTGA | 2912 |
| Lys90Asn | GCGACCTGCACGCGCACAAGCTTCGGGTGGACCCGGTCAAC | |
| AAGc-AAT | TTCAAGGTGAGCGGCGGGCCGGGAGCGATCTGGGTCGAG | |
| | CTCGACCCAGATCGCTCCCGGCCCGCCGCTCACCTTGAAGT | 2913 |
| | TGACCGGGTCCACCCGAAGCTTGTGCGCGTGCAGGTCGCTC | |
| | AGGGCGGACAGCGCGTTGGGCATGTCGTCCACGTGCGCC | |
| | GCGCACAAGCTTCGGGT | 2914 |
| | ACCCGAAGCTTGTGCGC | 2915 |
| Haemoglobin variant | GACGACATGCCCAACGCGCTGTCCGCCCTGAGCGACCTGCA | 2916 |
| Asp94His | CGCGCACAAGCTTCGGGTGGACCCGGTCAACTTCAAGGTGA | |
| gGAC-CAC | GCGGCGGGCCGGGAGCGATCTGGGTCGAGGGGCGAGATG | |
| | CATCTCGCCCCTCGACCCAGATCGCTCCCGGCCCGCCGCTC | 2917 |
| | ACCTTGAAGTTGACCGGGTCCACCCGAAGCTTGTGCGCGTG | |
| | CAGGTCGCTCAGGGCGGACAGCGCGTTGGGCATGTCGTC | |
| | TTCGGGTGGACCCGGTC | 2918 |
| | GACCGGGTCCACCCGAA | 2919 |
| Haemoglobin variant | ACATGCCCAACGCGCTGTCCGCCCTGAGCGACCTGCACGCG | 2920 |
| Pro95Leu | CACAAGCTTCGGGTGGACCCGGTCAACTTCAAGGTGAGCGG | |
| CCG-CTG | CGGGCCGGGAGCGATCTGGGTCGAGGGGCGAGATGGCGC | |
| | GCGCCATCTCGCCCCTCGACCCAGATCGCTCCCGGCCCGCC | 2921 |
| | GCTCACCTTGAAGTTGACCGGGTCCACCCGAAGCTTGTGCG | |
| | CGTGCAGGTCGCTCAGGGCGGACAGCGCGTTGGGCATGT | |
| | GGTGGACCCGGTCAACT | 2922 |
| | AGTTGACCGGGTCCACC | 2923 |
| Haemoglobin variant | TAGCGCAGGCGGCGGCTGCGGGCCTGGGCCGCACTGACCC | 2924 |
| Ser102Arg | TCTTCTCTGCACAGCTCCTAAGCCACTGCCTGCTGGTGACCC | |
| aAGC-CGC | TGGCCGCCCACCTCCCCGCCGAGTTCACCCCTGCGGTGC | |
| | GCACCGCAGGGGTGAACTCGGCGGGGAGGTGGGCGGCCAG | 2925 |
| | GGTCACCAGCAGGCAGTGGCTTAGGAGCTGTGCAGAGAAGA | |
| | GGGTCAGTGCGGCCCAGGCCCGCAGCCGCCGCCTGCGCTA | |
| | AGCTCCTAAGCCACTGC | 2926 |
| | GCAGTGGCTTAGGAGCT | 2927 |
| Haemoglobin H disease | GGCGGCGGCTGCGGGCCTGGGCCGCACTGACCCTCTTCTCT | 2928 |
| Cys104Tyr | GCACAGCTCCTAAGCCACTGCCTGCTGGTGACCCTGGCCGC | |
| TGC-TAC | CCACCTCCCCGCCGAGTTCACCCCTGCGGTGCACGCCTC | |
| | GAGGCGTGCACCGCAGGGGTGAACTCGGCGGGGAGGTGGG | 2929 |
| | CGGCCAGGGTCACCAGCAGGCAGTGGCTTAGGAGCTGTGCA | |
| | GAGAAGAGGGTCAGTGCGGCCCAGGCCCGCAGCCGCCGCC | |
| | AAGCCACTGCCTGCTGG | 2930 |
| | CCAGCAGGCAGTGGCTT | 2931 |
| Haemoglobin variant | CCGCACTGACCCTCTTCTCTGCACAGCTCCTAAGCCACTGCC | 2932 |
| Ala111Val | TGCTGGTGACCCTGGCCGCCCACCTCCCCGCCGAGTTCACC | |
| GCC-GTC | CCTGCGGTGCACGCCTCCCTGGACAAGTTCCTGGCTTC | |
| | GAAGCCAGGAACTTGTCCAGGGAGGCGTGCACCGCAGGGGT | 2933 |
| | GAACTCGGCGGGGAGGTGGGCGGCCAGGGTCACCAGCAGG | |
| | CAGTGGCTTAGGAGCTGTGCAGAGAAGAGGGTCAGTGCGG | |
| | CCTGGCCGCCCACCTCC | 2934 |
| | GGAGGTGGGCGGCCAGG | 2935 |
| Haemoglobin variant | TCCTAAGCCACTGCCTGCTGGTGACCCTGGCCGCCCACCTC | 2936 |
| Ala120Glu | CCCGCCGAGTTCACCCCTGCGGTGCACGCCTCCCTGGACAA | |
| GCG-GAG | GTTCCTGGCTTCTGTGAGCACCGTGCTGACCTCCAAATA | |
| | TATTTGGAGGTCAGCACGGTGCTCACAGAAGCCAGGAACTTG | 2937 |
| | TCCAGGGAGGCGTGCACCGCAGGGGTGAACTCGGCGGGGA | |
| | GGTGGGCGGCCAGGGTCACCAGCAGGCAGTGGCTTAGGA | |
| | CACCCCTGCGGTGCACG | 2938 |
| | CGTGCACCGCAGGGGTG | 2939 |
| Haemoglobin variant | CCACTGCCTGCTGGTGACCCTGGCCGCCCACCTCCCCGCCG | 2940 |
| His122Gln | AGTTCACCCCTGCGGTGCACGCCTCCCTGGACAAGTTCCTG | |
| CACg-CAG | GCTTCTGTGAGCACCGTGCTGACCTCCAAATACCGTTAA | |
| | TTAACGGTATTTGGAGGTCAGCACGGTGCTCACAGAAGCCAG | 2941 |
| | GAACTTGTCCAGGGAGGCGTGCACCGCAGGGGTGAACTCGG | |
| | CGGGGAGGTGGGCGGCCAGGGTCACCAGCAGGCAGTGG | |
| | GCGGTGCACGCCTCCCT | 2942 |
| | AGGGAGGCGTGCACCGC | 2943 |
| Haemoglobin variant | CACTGCCTGCTGGTGACCCTGGCCGCCCACCTCCCCGCCGA | 2944 |
| Ala123Ser | GTTCACCCCTGCGGTGCACGCCTCCCTGGACAAGTTCCTGG | |
| cGCC-TCC | CTTCTGTGAGCACCGTGCTGACCTCCAAATACCGTTAAG | |
| | CTTAACGGTATTTGGAGGTCAGCACGGTGCTCACAGAAGCCA | 2945 |
| | GGAACTTGTCCAGGGAGGCGTGCACCGCAGGGGTGAACTCG | |
| | GCGGGGAGGTGGGCGGCCAGGGTCACCAGCAGGCAGTG | |
| | CGGTGCACGCCTCCCTG | 2946 |
| | CAGGGAGGCGTGCACCG | 2947 |
| Thalassaemia alpha | TGCTGGTGACCCTGGCCGCCCACCTCCCCGCCGAGTTCACC | 2948 |
| Leu125Pro | CCTGCGGTGCACGCCTCCCTGGACAAGTTCCTGGCTTCTGTG | |
| CTG-CCG | AGCACCGTGCTGACCTCCAAATACCGTTAAGCTGGAGC | |
| | GCTCCAGCTTAACGGTATTTGGAGGTCAGCACGGTGCTCACA | 2949 |
| | GAAGCCAGGAACTTGTCCAGGGAGGCGTGCACCGCAGGGGT | |
| | GAACTCGGCGGGGAGGTGGGCGGCCAGGGTCACCAGCA | |
| | CGCCTCCCTGGACAAGT | 2950 |
| | ACTTGTCCAGGGAGGCG | 2951 |
| Haemoglobin variant | GCCCACCTCCCCGCCGAGTTCACCCCTGCGGTGCACGCCTC | 2952 |
| Ser131Pro | CCTGGACAAGTTCCTGGCTTCTGTGAGCACCGTGCTGACCTC | |
| tTCT-CCT | CAAATACCGTTAAGCTGGAGCCTCGGTAGCCGTTCCTC | |
| | GAGGAACGGCTACCGAGGCTCCAGCTTAACGGTATTTGGAG | 2953 |
| | GTCAGCACGGTGCTCACAGAAGCCAGGAACTTGTCCAGGGA | |
| | GGCGTGCACCGCAGGGGTGAACTCGGCGGGGAGGTGGGC | |
| | TCCTGGCTTCTGTGAGC | 2954 |
| | GCTCACAGAAGCCAGGA | 2955 |
| Haemoglobin variant | GAGTTCACCCCTGCGGTGCACGCCTCCCTGGACAAGTTCCT | 2956 |
| Leu136Met | GGCTTCTGTGAGCACCGTGCTGACCTCCAAATACCGTTAAGC | |
| gCTG-ATG | TGGAGCCTCGGTAGCCGTTCCTCCTGCCCGCTGGGCCT | |
| | AGGCCCAGCGGGCAGGAGGAACGGCTACCGAGGCTCCAGC | 2957 |
| | TTAACGGTATTTGGAGGTCAGCACGGTGCTCACAGAAGCCAG | |
| | GAACTTGTCCAGGGAGGCGTGCACCGCAGGGGTGAACTC | |
| | GCACCGTGCTGACCTCC | 2958 |
| | GGAGGTCAGCACGGTGC | 2959 |
| Haemoglobin variant | AGTTCACCCCTGCGGTGCACGCCTCCCTGGACAAGTTCCTG | 2960 |
| Leu136Pro | GCTTCTGTGAGCACCGTGCTGACCTCCAAATACCGTTAAGCT | |
| CTG-CCG | GGAGCCTCGGTAGCCGTTCCTCCTGCCCGCTGGGCCTC | |
| | GAGGCCCAGCGGGCAGGAGGAACGGCTACCGAGGCTCCAG | 2961 |
| | CTTAACGGTATTTGGAGGTCAGCACGGTGCTCACAGAAGCCA | |
| | GGAACTTGTCCAGGGAGGCGTGCACCGCAGGGGTGAACT | |
| | CACCGTGCTGACCTCCA | 2962 |
| | TGGAGGTCAGCACGGTG | 2963 |
| Haemoglobin variant | GTGCACGCCTCCCTGGACAAGTTCCTGGCTTCTGTGAGCACC | 2964 |
| Arg141Cys | GTGCTGACCTCCAAATACCGTTAAGCTGGAGCCTCGGTAGCC | |
| cCGT-TGT | GTTCCTCCTGCCCGCTGGGCCTCCCAACGGGCCCTCC | |
| | GGAGGGCCCGTTGGGAGGCCCAGCGGGCAGGAGGAACGGC | 2965 |
| | TACCGAGGCTCCAGCTTAACGGTATTTGGAGGTCAGCACGGT | |
| | GCTCACAGAAGCCAGGAACTTGTCCAGGGAGGCGTGCAC | |
| | CCAAATACCGTTAAGCT | 2966 |
| | AGCTTAACGGTATTTGG | 2967 |
| Haemoglobin variant | CACGCCTCCCTGGACAAGTTCCTGGCTTCTGTGAGCACCGTG | 2968 |
| Term142Gln | CTGACCTCCAAATACCGTTAAGCTGGAGCCTCGGTAGCCGTT | |
| tTAA-CAA | CCTCCTGCCCGCTGGGCCTCCCAACGGGCCCTCCTCC | |
| | GGAGGAGGGCCCGTTGGGAGGCCCAGCGGGCAGGAGGAAC | 2969 |
| | GGCTACCGAGGCTCCAGCTTAACGGTATTTGGAGGTCAGCA | |
| | CGGTGCTCACAGAAGCCAGGAACTTGTCCAGGGAGGCGTG | |
| | AATACCGTTAAGCTGGA | 2970 |
| | TCCAGCTTAACGGTATT | 2971 |
| Haemoglobin variant | CACGCCTCCCTGGACAAGTTCCTGGCTTCTGTGAGCACCGTG | 2972 |
| Term142Lys | CTGACCTCCAAATACCGTTAAGCTGGAGCCTCGGTAGCCGTT | |
| tTAA-AAA | CCTCCTGCCCGCTGGGCCTCCCAACGGGCCCTCCTCC | |
| | GGAGGAGGGCCCGTTGGGAGGCCCAGCGGGCAGGAGGAAC | 2973 |
| | GGCTACCGAGGCTCCAGCTTAACGGTATTTGGAGGTCAGCA | |
| | CGGTGCTCACAGAAGCCAGGAACTTGTCCAGGGAGGCGTG | |
| | AATACCGTTAAGCTGGA | 2974 |
| | TCCAGCTTAACGGTATT | 2975 |
| Haemoglobin variant | CGCCTCCCTGGACAAGTTCCTGGCTTCTGTGAGCACCGTGCT | 2976 |
| Term142Tyr | GACCTCCAAATACCGTTAAGCTGGAGCCTCGGTAGCCGTTCC | |
| TAAg-TAT | TCCTGCCCGCTGGGCCTCCCAACGGGCCCTCCTCCCC | |
| | GGGGAGGAGGGCCCGTTGGGAGGCCCAGCGGGCAGGAGGA | 2977 |
| | ACGGCTACCGAGGCTCCAGCTTAACGGTATTTGGAGGTCAGC | |
| | ACGGTGCTCACAGAAGCCAGGAACTTGTCCAGGGAGGCG | |
| | TACCGTTAAGCTGGAGC | 2978 |
| | GCTCCAGCTTAACGGTA | 2979 |

### EXAMPLE 18

### Human Mismatch Repair—MLH1

The human MLH1 gene is homologous to the bacterial mutL gene, which is involved in mismatch repair. Mutations in the MLH1 gene have been identified in many individuals with hereditary nonpolyposis colorectal cancer (HNPCC). Mutations in the MLH1 gene are also implicated in predisposition to a variety of cancers associated with, for example, Muir-Torre syndrome and Turcot syndrome. The attached table discloses the correcting oligonucleotide base sequences for the MLH1 oligonucleotides of the invention.

TABLE 20
MLH1 Mutations and Genome-Correcting Oligos

| Clinical Phenotype & | | SEQ ID |
|---|---|---|
| Mutation | Correcting Oligos | NO: |
| Non-polyposis | TTGGCTGAAGGCACTTCCGTTGAGCATCTAGACGTTTCCTTG | 2980 |
| colorectal cancer | GCTCTTCTGGCGCCAAAATGTCGTTCGTGGCAGGGGTTATTC | |
| Met1Arg | GGCGGCTGGACGAGACAGTGGTGAACCGCATCGCGGC | |
| ATG-AGG | GCCGCGATGCGGTTCACCACTGTCTCGTCCAGCCGCCGAAT | 2981 |
| | AACCCCTGCCACGAACGACATTTTGGCGCCAGAAGAGCCAA | |
| | GGAAACGTCTAGATGCTCAACGGAAGTGCCTTCAGCCAA | |
| | CGCCAAAATGTCGTTCG | 2982 |
| | CGAACGACATTTTGGCG | 2983 |
| Non-polyposis | TTGGCTGAAGGCACTTCCGTTGAGCATCTAGACGTTTCCTTG | 2984 |
| colorectal cancer | GCTCTTCTGGCGCCAAAATGTCGTTCGTGGCAGGGGTTATTC | |
| Met1Lys | GGCGGCTGGACGAGACAGTGGTGAACCGCATCGCGGC | |
| ATG-AAG | GCCGCGATGCGGTTCACCACTGTCTCGTCCAGCCGCCGAAT | 2985 |
| | AACCCCTGCCACGAACGACATTTTGGCGCCAGAAGAGCCAA | |
| | GGAAACGTCTAGATGCTCAACGGAAGTGCCTTCAGCCAA | |
| | CGCCAAAATGTCGTTCG | 2986 |
| | CGAACGACATTTTGGCG | 2987 |
| Non-polyposis | TGGTGAACCGCATCGCGGCGGGGGAAGTTATCCAGCGGCCA | 2988 |
| colorectal cancer | GCTAATGCTATCAAAGAGATGATTGAGAACTGGTACGGAGGG | |
| Met35Arg | AGTCGAGCCGGGCTCACTTAAGGGCTACGACTTAACGG | |
| ATG-AGG | CCGTTAAGTCGTAGCCCTTAAGTGAGCCCGGCTCGACTCCCT | 2989 |
| | CCGTACCAGTTCTCAATCATCTCTTTGATAGCATTAGCTGGCC | |
| | GCTGGATAACTTCCCCCGCCGCGATGCGGTTCACCA | |
| | CAAAGAGATGATTGAGA | 2990 |
| | TCTCAATCATCTCTTTG | 2991 |
| Non-polyposis | TAGAGTAGTTGCAGACTGATAAATTATTTTCTGTTTGATTTGCC | 2992 |
| colorectal cancer | AGTTTAGATGCAAAATCCACAAGTATTCAAGTGATTGTTAAAG | |
| Ser44Phe | AGGGAGGCCTGAAGTTGATTCAGATCCAAGACAA | |
| TCC-TTC | TTGTCTTGGATCTGAATCAACTTCAGGCCTCCCTCTTTAACAA | 2993 |
| | TCACTTGAATACTTGTGGATTTTGCATCTAAACTGGCAAATCA | |
| | AACAGAAAATAATTTATCAGTCTGCAACTACTCTA | |
| | TGCAAAATCCACAAGTA | 2994 |
| | TACTTGTGGATTTTGCA | 2995 |
| Non-polyposis | GCAAAATCCACAAGTATTCAAGTGATTGTTAAAGAGGGAGGC | 2996 |
| colorectal cancer | CTGAAGTTGATTCAGATCCAAGACAATGGCACCGGGATCAGG | |
| Gln62Lys | GTAAGTAAAACCTCAAAGTAGCAGGATGTTTGTGCGC | |
| CAA-AAA | GCGCACAAACATCCTGCTACTTTGAGGTTTTACTTACCCTGAT | 2997 |
| | CCCGGTGCCATTGTCTTGGATCTGAATCAACTTCAGGCCTCC | |
| | CTCTTTAACAATCACTTGAATACTTGTGGATTTTGC | |
| | TTCAGATCCAAGACAAT | 2998 |
| | ATTGTCTTGGATCTGAA | 2999 |
| Non-polyposis | GCAAAATCCACAAGTATTCAAGTGATTGTTAAAGAGGGAGGC | 3000 |
| colorectal cancer | CTGAAGTTGATTCAGATCCAAGACAATGGCACCGGGATCAGG | |
| Gln62Term | GTAAGTAAAACCTCAAAGTAGCAGGATGTTTGTGCGC | |
| CAA-TAA | GCGCACAAACATCCTGCTACTTTGAGGTTTTACTTACCCTGAT | 3001 |
| | CCCGGTGCCATTGTCTTGGATCTGAATCAACTTCAGGCCTCC | |
| | CTCTTTAACAATCACTTGAATACTTGTGGATTTTGC | |
| | TTCAGATCCAAGACAAT | 3002 |
| | ATTGTCTTGGATCTGAA | 3003 |
| Non-polyposis | CCACAAGTATTCAAGTGATTGTTAAAGAGGGAGGCCTGAAGT | 3004 |
| colorectal cancer | TGATTCAGATCCAAGACAATGGCACCGGGATCAGGGTAAGTA | |
| Asn64Ser | AAACCTCAAAGTAGCAGGATGTTTGTGCGCTTCATGG | |
| AAT-AGT | CCATGAAGCGCACAAACATCCTGCTACTTTGAGGTTTTACTTA | 3005 |
| | CCCTGATCCCGGTGCCATTGTCTTGGATCTGAATCAACTTCA | |
| | GGCCTCCCTCTTTAACAATCACTTGAATACTTGTGG | |
| | CCAAGACAATGGCACCG | 3006 |
| | CGGTGCCATTGTCTTGG | 3007 |
| Non-polyposis | ATTCAAGTGATTGTTAAAGAGGGAGGCCTGAAGTTGATTCAG | 3008 |
| colorectal cancer | ATCCAAGACAATGGCACCGGGATCAGGGTAAGTAAAACCTCA | |
| Gly67Arg | AAGTAGCAGGATGTTTGTGCGCTTCATGGAAGAGTCA | |
| GGG-AGG | TGACTCTTCCATGAAGCGCACAAACATCCTGCTACTTTGAGGT | 3009 |
| | TTTACTTACCCTGATCCCGGTGCCATTGTCTTGGATCTGAATC | |
| | AACTTCAGGCCTCCCTCTTTAACAATCACTTGAAT | |
| | ATGGCACCGGGATCAGG | 3010 |
| | CCTGATCCCGGTGCCAT | 3011 |
| Non-polyposis | ATTCAAGTGATTGTTAAAGAGGGAGGCCTGAAGTTGATTCAG | 3012 |
| colorectal cancer | ATCCAAGACAATGGCACCGGGATCAGGGTAAGTAAAACCTCA | |
| Gly67Arg | AAGTAGCAGGATGTTTGTGCGCTTCATGGAAGAGTCA | |
| GGG-CGG | TGACTCTTCCATGAAGCGCACAAACATCCTGCTACTTTGAGGT | 3013 |
| | TTTACTTACCCTGATCCCGGTGCCATTGTCTTGGATCTGAATC | |
| | AACTTCAGGCCTCCCTCTTTAACAATCACTTGAAT | |
| | ATGGCACCGGGATCAGG | 3014 |
| | CCTGATCCCGGTGCCAT | 3015 |
| Non-polyposis | ATTCAAGTGATTGTTAAAGAGGGAGGCCTGAAGTTGATTCAG | 3016 |
| colorectal cancer | ATCCAAGACAATGGCACCGGGATCAGGGTAAGTAAAACCTCA | |
| Gly67Trp | AAGTAGCAGGATGTTTGTGCGCTTCATGGAAGAGTCA | |
| GGG-TGG | TGACTCTTCCATGAAGCGCACAAACATCCTGCTACTTTGAGGT | 3017 |
| | TTTACTTACCCTGATCCCGGTGCCATTGTCTTGGATCTGAATC | |
| | AACTTCAGGCCTCCCTCTTTAACAATCACTTGAAT | |
| | ATGGCACCGGGATCAGG | 3018 |
| | CCTGATCCCGGTGCCAT | 3019 |
| Non-polyposis | GTAACATGATTATTTACTCATCTTTTTGGTATCTAACAGAAAGA | 3020 |
| colorectal cancer | AGATCTGGATATTGTATGTGAAAGGTTCACTACTAGTAAACTG | |
| Cys77Arg | CAGTCCTTTGAGGATTTAGCCAGTATTTCTACCT | |
| TGT-CGT | AGGTAGAAATACTGGCTAAATCCTCAAAGGACTGCAGTTTACT | 3021 |
| | AGTAGTGAACCTTTCACATACAATATCCAGATCTTCTTTCTGTT | |
| | AGATACCAAAAAGATGAGTAAATAATCATGTTAC | |
| | ATATTGTATGTGAAAGG | 3022 |
| | CCTTTCACATACAATAT | 3023 |
| Non-polyposis | TAACATGATTATTTACTCATCTTTTTGGTATCTAACAGAAAGAA | 3024 |
| colorectal cancer | GATCTGGATATTGTATGTGAAAGGTTCACTACTAGTAAACTGC | |
| Cys77Tyr | AGTCCTTTGAGGATTTAGCCAGTATTTCTACCTA | |
| TGT-TAT | TAGGTAGAAATACTGGCTAAATCCTCAAAGGACTGCAGTTTAC | 3025 |
| | TAGTAGTGAACCTTTCACATACAATATCCAGATCTTCTTTCTGT | |
| | TAGATACCAAAAAGATGAGTAAATAATCATGTTA | |
| | TATTGTATGTGAAAGGT | 3026 |
| | ACCTTTCACATACAATA | 3027 |
| Non-polyposis | CTGGATATTGTATGTGAAAGGTTCACTACTAGTAAACTGCAGT | 3028 |
| colorectal cancer | CCTTTGAGGATTTAGCCAGTATTTCTACCTATGGCTTTCGAGG | |
| Ser93Gly | TGAGGTAAGCTAAAGATTCAAGAAATGTGTAAAAT | |
| AGT-GGT | ATTTTACACATTTCTTGAATCTTTAGCTTACCTCACCTCGAAAG | 3029 |
| | CCATAGGTAGAAATACTGGCTAAATCCTCAAAGGACTGCAGT | |
| | TTACTAGTAGTGAACCTTTCACATACAATATCCAG | |
| | ATTTAGCCAGTATTTCT | 3030 |
| | AGAAATACTGGCTAAAT | 3031 |
| Non-polyposis | TTCACTACTAGTAAACTGCAGTCCTTTGAGGATTTAGCCAGTA | 3032 |
| colorectal cancer | TTTCTACCTATGGCTTTCGAGGTGAGGTAAGCTAAAGATTCAA | |
| Arg100Term | GAAATGTGTAAAATATCCTCCTGTGATGACATTGT | |
| CGA-TGA | ACAATGTCATCACAGGAGGATATTTTACACATTTCTTGAATCTT | 3033 |
| | TAGCTTACCTCACCTCGAAAGCCATAGGTAGAAATACTGGCT | |
| | AAATCCTCAAAGGACTGCAGTTTACTAGTAGTGAA | |
| | ATGGCTTTCGAGGTGAG | 3034 |
| | CTCACCTCGAAAGCCAT | 3035 |
| Non-polyposis | ACCCAGCAGTGAGTTTTTCTTTCAGTCTATTTTCTTTTCTTCCT | 3036 |
| colorectal cancer | TAGGCTTTGGCCAGCATAAGCCATGTGGCTCATGTTACTATTA | |
| Ile107Arg | CAACGAAAACAGCTGATGGAAAGTGTGCATACAG | |
| ATA-AGA | CTGTATGCACACTTTCCATCAGCTGTTTTCGTTGTAATAGTAA | 3037 |
| | CATGAGCCACATGGCTTATGCTGGCCAAAGCCTAAGGAAGAA | |
| | AAGAAAATAGACTGAAAGAAAAACTCACTGCTGGGT | |
| | GGCCAGCATAAGCCATG | 3038 |
| | CATGGCTTATGCTGGCC | 3039 |
| Non-polyposis | TTTCTTTTCTTCCTTAGGCTTTGGCCAGCATAAGCCATGTGGC | 3040 |
| colorectal cancer | TCATGTTACTATTACAACGAAAACAGCTGATGGAAAGTGTGCA | |
| Thr117Arg | TACAGGTATAGTGCTGACTTCTTTTACTCATATAT | |
| ACG-AGG | ATATATGAGTAAAAGAAGTCAGCACTATACCTGTATGCACACT | 3041 |
| | TTCCATCAGCTGTTTTCGTTGTAATAGTAACATGAGCCACATG | |
| | GCTTATGCTGGCCAAAGCCTAAGGAAGAAAAGAAA | |
| | TATTACAACGAAAACAG | 3042 |
| | CTGTTTTCGTTGTAATA | 3043 |
| Non-polyposis | TTTCTTTTCTTCCTTAGGCTTTGGCCAGCATAAGCCATGTGGC | 3044 |
| colorectal cancer | TCATGTTACTATTACAACGAAAACAGCTGATGGAAAGTGTGCA | |
| Thr117Met | TACAGGTATAGTGCTGACTTCTTTTACTCATATAT | |
| ACG-ATG | ATATATGAGTAAAAGAAGTCAGCACTATACCTGTATGCACACT | 3045 |
| | TTCCATCAGCTGTTTTCGTTGTAATAGTAACATGAGCCACATG | |
| | GCTTATGCTGGCCAAAGCCTAAGGAAGAAAAGAAA | |
| | TATTACAACGAAAACAG | 3046 |
| | CTGTTTTCGTTGTAATA | 3047 |
| Non-polyposis | TCTATCTCTCTACTGGATATTAATTTGTTATATTTTCTCATTAGA | 3048 |
| colorectal cancer | GCAAGTTACTCAGATGGAAAACTGAAAGCCCCTCCTAAACCA | |
| Gly133Term | TGTGCTGGCAATCAAGGGACCCAGATCACGGTAA | |
| GGA-TGA | TTACCGTGATCTGGGTCCCTTGATTGCCAGCACATGGTTTAG | 3049 |
| | GAGGGGCTTTCAGTTTTCCATCTGAGTAACTTGCTCTAATGAG | |
| | AAAATATAACAAATTAATATCCAGTAGAGAGATAGA | |
| | ACTCAGATGGAAAACTG | 3050 |
| | CAGTTTTCCATCTGAGT | 3051 |
| Non-polyposis | TAGTGTGTGTTTTTGGCAACTCTTTTCTTACTCTTTTGTTTTTC | 3052 |
| colorectal cancer | TTTTCCAGGTATTCAGTACACAATGCAGGCATTAGTTTCTCAG | |
| Val185Gly | TTAAAAAAGTAAGTTCTTGGTTTATGGGGGATGG | |
| GTA-GGA | CCATCCCCCATAAACCAAGAACTTACTTTTTTAACTGAGAAAC | 3053 |
| | TAATGCCTGCATTGTGTACTGAATACCTGGAAAAGAAAAACAA | |
| | AAGAGTAAGAAAAGAGTTGCCAAAAACACACACTA | |
| | GTATTCAGTACACAATG | 3054 |
| | CATTGTGTACTGAATAC | 3055 |
| Non-polyposis | TTTCTTACTCTTTTGTTTTTCTTTTCCAGGTATTCAGTACACAA | 3056 |
| colorectal cancer | TGCAGGCATTAGTTTCTCAGTTAAAAAAGTAAGTTCTTGGTTT | |
| Ser193Pro | ATGGGGGATGGTTTTGTTTTATGAAAAGAAAAAA | |
| TCA-CCA | TTTTTTCTTTTCATAAAACAAAACCATCCCCCATAAACCAAGAA | 3057 |
| | CTTACTTTTTTAACTGAGAAACTAATGCCTGCATTGTGTACTG | |
| | AATACCTGGAAAAGAAAAACAAAAGAGTAAGAAA | |
| | TTAGTTTCTCAGTTAAA | 3058 |
| | TTTAACTGAGAAACTAA | 3059 |
| Non-polyposis | TTTGTTTATCAGCAAGGAGAGACAGTAGCTGATGTTAGGACA | 3060 |
| colorectal cancer | CTACCCAATGCCTCAACCGTGGACAATATTCGCTCCATCTTTG | |
| Val213Met | GAAATGCTGTTAGTCGGTATGTCGATAACCTATATA | |
| GTG-ATG | TATATAGGTTATCGACATACCGACTAACAGCATTTCCAAAGAT | 3061 |
| | GGAGCGAATATTGTCCACGGTTGAGGCATTGGGTAGTGTCCT | |
| | AACATCAGCTACTGTCTCTCCTTGCTGATAAACAAA | |
| | CCTCAACCGTGGACAAT | 3062 |
| | ATTGTCCACGGTTGAGG | 3063 |
| Non-polyposis | CAAGGAGAGACAGTAGCTGATGTTAGGACACTACCCAATGCC | 3064 |
| colorectal cancer | TCAACCGTGGACAATATTCGCTCCATCTTTGGAAATGCTGTTA | |
| Arg217Cys | GTCGGTATGTCGATAACCTATATAAAAAAATCTTTT | |
| CGC-TGC | AAAAGATTTTTTTATATAGGTTATCGACATACCGACTAACAGC | 3065 |
| | ATTTCCAAAGATGGAGCGAATATTGTCCACGGTTGAGGCATT | |
| | GGGTAGTGTCCTAACATCAGCTACTGTCTCTCCTTG | |
| | ACAATATTCGCTCCATC | 3066 |
| | GATGGAGCGAATATTGT | 3067 |
| Non-polyposis | GAGACAGTAGCTGATGTTAGGACACTACCCAATGCCTCAACC | 3068 |
| colorectal cancer | GTGGACAATATTCGCTCCATCTTTGGAAATGCTGTTAGTCGGT | |
| Ile219Val | ATGTCGATAACCTATATAAAAAAATCTTTTACATTT | |
| ATC-GTC | AAATGTAAAAGATTTTTTTATATAGGTTATCGACATACCGACTA | 3069 |
| | ACAGCATTTCCAAAGATGGAGCGAATATTGTCCACGGTTGAG | |
| | GCATTGGGTAGTGTCCTAACATCAGCTACTGTCTC | |
| | TTCGCTCCATCTTTGGA | 3070 |
| | TCCAAAGATGGAGCGAA | 3071 |
| Non-polyposis | CTAATAGAGAACTGATAGAAATTGGATGTGAGGATAAAACCCT | 3072 |
| colorectal cancer | AGCCTTCAAAATGAATGGTTACATATCCAATGCAAACTACTCA | |
| Gly244Asp | GTGAAGAAGTGCATCTTCTTACTCTTCATCAACCG | |
| GGT-GAT | CGGTTGATGAAGAGTAAGAAGATGCACTTCTTCACTGAGTAG | 3073 |
| | TTTGCATTGGATATGTAACCATTCATTTTGAAGGCTAGGGTTT | |
| | TATCCTCACATCCAATTTCTATCAGTTCTCTATTAG | |
| | AATGAATGGTTACATAT | 3074 |
| | ATATGTAACCATTCATT | 3075 |
| Non-polyposis | GATGTGAGGATAAAACCCTAGCCTTCAAAATGAATGGTTACAT | 3076 |
| colorectal cancer | ATCCAATGCAAACTACTCAGTGAAGAAGTGCATCTTCTTACTC | |
| Ser252Term | TTCATCAACCGTAAGTTAAAAAGAACCACATGGGA | |
| TCA-TAA | TCCCATGTGGTTCTTTTTAACTTACGGTTGATGAAGAGTAAGA | 3077 |
| | AGATGCACTTCTTCACTGAGTAGTTTGCATTGGATATGTAACC | |
| | ATTCATTTTGAAGGCTAGGGTTTTATCCTCACATC | |
| | AAACTACTCAGTGAAGA | 3078 |
| | TCTTCACTGAGTAGTTT | 3079 |
| Non-polyposis | CACCCCTCAGGACAGTTTTGAACTGGTTGCTTTCTTTTTATTG | 3080 |
| colorectal cancer | TTTAGATCGTCTGGTAGAATCAACTTCCTTGAGAAAAGCCATA | |
| Glu268Gly | GAAACAGTGTATGCAGCCTATTTGCCCAAAAACAC | |
| GAA-GGA | GTGTTTTTGGGCAAATAGGCTGCATACACTGTTTCTATGGCTT | 3081 |
| | TTCTCAAGGAAGTTGATTCTACCAGACGATCTAAACAATAAAA | |
| | AGAAAGCAACCAGTTCAAAACTGTCCTGAGGGGTG | |
| | TCTGGTAGAATCAACTT | 3082 |
| | AAGTTGATTCTACCAGA | 3083 |
| Non-polyposis | CCCTCAGGACAGTTTTGAACTGGTTGCTTTCTTTTTATTGTTTA | 3084 |
| colorectal cancer | GATCGTCTGGTAGAATCAACTTCCTTGAGAAAAGCCATAGAAA | |
| Ser269Term | CAGTGTATGCAGCCTATTTGCCCAAAAACACACA | |
| TCA-TGA | TGTGTGTTTTTGGGCAAATAGGCTGCATACACTGTTTCTATGG | 3085 |
| | CTTTTCTCAAGGAAGTTGATTCTACCAGACGATCTAAACAATA | |
| | AAAAGAAAGCAACCAGTTCAAAACTGTCCTGAGGG | |
| | GGTAGAATCAACTTCCT | 3086 |
| | AGGAAGTTGATTCTACC | 3087 |
| Non-polyposis | CTTTTTCTCCCCCTCCCACTATCTAAGGTAATTGTTCTCTCTTA | 3088 |
| colorectal cancer | TTTTCCTGACAGTTTAGAAATCAGTCCCCAGAATGTGGATGTT | |
| Glu297Term | AATGTGCACCCCACAAAGCATGAAGTTCACTTCC | |
| GAA-TAA | GGAAGTGAACTTCATGCTTTGTGGGGTGCACATTAACATCCA | 3089 |
| | CATTCTGGGGACTGATTTCTAAACTGTCAGGAAAATAAGAGA | |
| | GAACAATTACCTTAGATAGTGGGAGGGGGAGAAAAAG | |
| | ACAGTTTAGAAATCAGT | 3090 |
| | ACTGATTTCTAAACTGT | 3091 |
| Non-polyposis | CTCCCACTATCTAAGGTAATTGTTCTCTCTTATTTTCCTGACAG | 3092 |
| colorectal cancer | TTTAGAAATCAGTCCCCAGAATGTGGATGTTAATGTGCACCCC | |
| Gln301Term | ACAAAGCATGAAGTTCACTTCCTGCACGAGGAGA | |
| CAG-TAG | TCTCCTCGTGCAGGAAGTGAACTTCATGCTTTGTGGGGTGCA | 3093 |
| | CATTAACATCCACATTCTGGGGACTGATTTCTAAACTGTCAGG | |
| | AAAATAAGAGAGAACAATTACCTTAGATAGTGGGAG | |
| | TCAGTCCCCAGAATGTG | 3094 |
| | CACATTCTGGGGACTGA | 3095 |
| Non-polyposis | ATGTGCACCCCACAAAGCATGAAGTTCACTTCCTGCACGAGG | 3096 |
| colorectal cancer | AGAGCATCCTGGAGCGGGTGCAGCAGCACATCGAGAGCAAG | |
| Val326Ala | CTCCTGGGCTCCAATTCCTCCAGGATGTACTTCACCCA | |
| GTG-GCG | TGGGTGAAGTACATCCTGGAGGAATTGGAGCCCAGGAGCTT | 3097 |
| | GCTCTCGATGTGCTGCTGCACCCGCTCCAGGATGCTCTCCTC | |
| | GTGCAGGAAGTGAACTTCATGCTTTGTGGGGTGCACAT | |
| | GGAGCGGGTGCAGCAGC | 3098 |
| | GCTGCTGCACCCGCTCC | 3099 |
| Non-polyposis | CCACAAAGCATGAAGTTCACTTCCTGCACGAGGAGAGCATCC | 3100 |
| colorectal cancer | TGGAGCGGGTGCAGCAGCACATCGAGAGCAAGCTCCTGGGC | |
| His329Pro | TCCAATTCCTCCAGGATGTACTTCACCCAGGTCAGGGC | |
| CAC-CCC | GCCCTGACCTGGGTGAAGTACATCCTGGAGGAATTGGAGCC | 3101 |
| | CAGGAGCTTGCTCTCGATGTGCTGCTGCACCCGCTCCAGGAT | |
| | GCTCTCCTCGTGCAGGAAGTGAACTTCATGCTTTGTGG | |
| | GCAGCAGCACATCGAGA | 3102 |
| | TCTCGATGTGCTGCTGC | 3103 |
| Non-polyposis | CAAGTCTGACCTCGTCTTCTACTTCTGGAAGTAGTGATAAGGT | 3104 |
| colorectal cancer | CTATGCCCACCAGATGGTTCGTACAGATTCCCGGGAACAGAA | |
| Val384Asp | GCTTGATGCATTTCTGCAGCCTCTGAGCAAACCCCT | |
| GTT-GAT | AGGGGTTTGCTCAGAGGCTGCAGAAATGCATCAAGCTTCTGT | 3105 |
| | TCCCGGGAATCTGTACGAACCATCTGGTGGGCATAGACCTTA | |
| | TCACTACTTCCAGAAGTAGAAGACGAGGTCAGACTTG | |
| | CCAGATGGTTCGTACAG | 3106 |
| | CTGTACGAACCATCTGG | 3107 |
| Non-polyposis | AGTGGCAGGGCTAGGCAGCAAGATGAGGAGATGCTTGAACT | 3108 |
| colorectal cancer | CCCAGCCCCTGCTGAAGTGGCTGCCAAAAATCAGAGCTTGGA | |
| Ala441Thr | GGGGGATACAACAAAGGGGACTTCAGAAATGTCAGAGA | |
| GCT-ACT | TCTCTGACATTTCTGAAGTCCCCTTTGTTGTATCCCCCTCCAA | 3109 |
| | GCTCTGATTTTTGGCAGCCACTTCAGCAGGGGCTGGGAGTTC | |
| | AAGCATCTCCTCATCTTGCTGCCTAGCCCTGCCACT | |
| | CTGAAGTGGCTGCCAAA | 3110 |
| | TTTGGCAGCCACTTCAG | 3111 |
| Non-polyposis | CTTCATTGCAGAAAGAGACATCGGGAAGATTCTGATGTGGAA | 3112 |
| colorectal cancer | ATGGTGGAAGATGATTCCCGAAAGGAAATGACTGCAGCTTGT | |
| Arg487Term | ACCCCCCGGAGAAGGATCATTAACCTCACTAGTGTTT | |
| CGA-TGA | AAACACTAGTGAGGTTAATGATCCTTCTCCGGGGGGTACAAG | 3113 |
| | CTGCAGTCATTTCCTTTCGGGAATCATCTTCCACCATTTCCAC | |
| | ATCAGAATCTTCCCGATGTCTCTTTCTGCAATGAAG | |
| | ATGATTCCCGAAAGGAA | 3114 |
| | TTCCTTTCGGGAATCAT | 3115 |
| Non-polyposis | AGACATCGGGTAAGATTCTGATGTGGAAATGGTGGAAGATGAT | 3116 |
| colorectal cancer | TCCCGAAAGGAAATGACTGCAGCTTGTACCCCCCGGAGAAG | |
| Ala492Thr | GATCATTAACCTCACTAGTGTTTTGAGTCTCCAGGAAG | |
| GCA-ACA | CTTCCTGGAGACTCAAAACACTAGTGAGGTTAATGATCCTTCT | 3117 |
| | CCGGGGGGTACAAGCTGCAGTCATTTCCTTTCGGGAATCATC | |
| | TTCCACCATTTCCACATCAGAATCTTCCCGATGTCT | |
| | AAATGACTGCAGCTTGT | 3118 |
| | ACAAGCTGCAGTCATTT | 3119 |
| Non-polyposis | CCCGAAAGGAAATGACTGCAGCTTGTACCCCCCGGAGAAGG | 3120 |
| colorectal cancer | ATCATTAACCTCACTAGTGTTTTGAGTCTCCAGGAAGAAATTA | |
| Val506Ala | ATGAGCAGGGACATGAGGGTACGTAAACGCTGTGGCC | |
| GTT-GCT | GGCCACAGCGTTTACGTACCCTCATGTCCCTGCTCATTAATTT | 3121 |
| | CTTCCTGGAGACTCAAAACACTAGTGAGGTTAATGATCCTTCT | |
| | CCGGGGGGTACAAGCTGCAGTCATTTCCTTTCGGG | |
| | CACTAGTGTTTTGAGTC | 3122 |
| | GACTCAAAACACTAGTG | 3123 |
| Non-polyposis | GGGAGATGTTGCATAACCACTCCTTCGTGGGCTGTGTGAATC | 3124 |
| colorectal cancer | CTCAGTGGGCCTTGGCACAGCATCAAACCAAGTTATACCTTC | |
| Gln542Leu | TCAACACCACCAAGCTTAGGTAAATCAGCTGAGTGTG | |
| CAG-CTG | CACACTCAGCTGATTTACCTAAGCTTGGTGGTGTTGAGAAGG | 3125 |
| | TATAACTTGGTTTGATGCTGTGCCAAGGCCCACTGAGGATTC | |
| | ACACAGCCCACGAAGGAGTGGTTATGCAACATCTCCC | |
| | CTTGGCACAGCATCAAA | 3126 |
| | TTTGATGCTGTGCCAAG | 3127 |
| Non-polyposis | CCTTCGTGGGCTGTGTGAATCCTCAGTGGGCCTTGGCACAG | 3128 |
| colorectal cancer | CATCAAACCAAGTTATACCTTCTCAACACCACCAAGCTTAGGT | |
| Leu549Pro | AAATCAGCTGAGTGTGTGAACAAGCAGAGCTACTACA | |
| CTT-CCT | TGTAGTAGCTCTGCTTGTTCACACACTCAGCTGATTTACCTAA | 3129 |
| | GCTTGGTGGTGTTGAGAAGGTATAACTTGGTTTGATGCTGTG | |
| | CCAAGGCCCACTGAGGATTCACACAGCCCACGAAGG | |
| | GTTATACCTTCTCAACA | 3130 |
| | TGTTGAGAAGGTATAAC | 3131 |
| Non-polyposis | TGGGCTGTGTGAATCCTCAGTGGGCCTTGGCACAGCATCAAA | 3132 |
| colorectal cancer | CCAAGTTATACCTTCTCAACACCACCAAGCTTAGGTAAATCAG | |
| Asn551Thr | CTGAGTGTGTGAACAAGCAGAGCTACTACAACAATG | |
| AAC-ACC | CATTGTTGTAGTAGCTCTGCTTGTTCACACACTCAGCTGATTT | 3133 |
| | ACCTAAGCTTGGTGGTGTTGAGAAGGTATAACTTGGTTTGAT | |
| | GCTGTGCCAAGGCCCACTGAGGATTCACACAGCCCA | |
| | CCTTCTCAACACCACCA | 3134 |
| | TGGTGGTGTTGAGAAGG | 3135 |
| Non-polyposis | ATGAATTCAGCTTTTCCTTAAAGTCACTTCATTTTTATTTTCAG | 3136 |
| colorectal cancer | TGAAGAACTGTTCTACCAGATACTCATTTATGATTTTGCCAATT | |
| Gln562Term | TTGGTGTTCTCAGGTTATCGGTAAGTTTAGATC | |
| CAG-TAG | GATCTAAACTTACCGATAACCTGAGAACACCAAAATTGGCAAA | 3137 |
| | ATCATAAATGAGTATCTGGTAGAACAGTTCTTCACTGAAAATA | |
| | AAAATGAAGTGACTTTAAGGAAAAGCTGAATTCAT | |
| | TGTTCTACCAGATACTC | 3138 |
| | GAGTATCTGGTAGAACA | 3139 |
| Non-polyposis | GCTTTTCCTTAAAGTCACTTCATTTTTATTTTCAGTGAAGAACT | 3140 |
| colorectal cancer | GTTCTACCAGATACTCATTTATGATTTTGCCAATTTTGGTGTTC | |
| Ile565Phe | TCAGGTTATCGGTAAGTTTAGATCCTTTTCACT | |
| ATT-TTT | AGTGAAAAGGATCTAAACTTACCGATAACCTGAGAACACCAAA | 3141 |
| | ATTGGCAAAATCATAAATGAGTATCTGGTAGAACAGTTCTTCA | |
| | CTGAAAATAAAAATGAAGTGACTTTAAGGAAAAGC | |
| | AGATACTCATTTATGAT | 3142 |
| | ATCATAAATGAGTATCT | 3143 |
| Non-polyposis | TTTTCAGTGAAGAACTGTTCTACCAGATACTCATTTATGATTTT | 3144 |
| colorectal cancer | GCCAATTTTGGTGTTCTCAGGTTATCGGTAAGTTTAGATCCTT | |
| Leu574Pro | TTCACTTCTGAAATTTCAACTGATCGTTTCTGAA | |
| CTC-CCC | TTCAGAAACGATCAGTTGAAATTTCAGAAGTGAAAAGGATCTA | 3145 |
| | AACTTACCGATAACCTGAGAACACCAAAATTGGCAAAATCATA | |
| | AATGAGTATCTGGTAGAACAGTTCTTCACTGAAAA | |
| | TGGTGTTCTCAGGTTAT | 3146 |
| | ATAACCTGAGAACACCA | 3147 |
| Non-polyposis | TGGATGCTCCGTTAAAGCTTGCTCCTTCATGTTCTTGCTTCTT | 3148 |
| colorectal cancer | CCTAGGAGCCAGCACCGCTCTTTGACCTTGCCATGCTTGCCT | |
| Leu582Val | TAGATAGTCCAGAGAGTGGCTGGACAGAGGAAGATG | |
| CTC-GTC | CATCTTCCTCTGTCCAGCCACTCTCTGGACTATCTAAGGCAA | 3149 |
| | GCATGGCAAGGTCAAAGAGCGGTGCTGGCTCCTAGGAAGAA | |
| | GCAAGAACATGAAGGAGCAAGCTTTAACGGAGCATCCA | |
| | CAGCACCGCTCTTTGAC | 3150 |
| | GTCAAAGAGCGGTGCTG | 3151 |
| Non-polyposis | TGCTTGCCTTAGATAGTCCAGAGAGTGGCTGGACAGAGGAAG | 3152 |
| colorectal cancer | ATGGTCCCAAAGAAGGACTTGCTGAATACATTGTTGAGTTTCT | |
| Leu607His | GAAGAAGAAGGCTGAGATGCTTGCAGACTATTTCTC | |
| CTT-CAT | GAGAAATAGTCTGCAAGCATCTCAGCCTTCTTCTTCAGAAACT | 3153 |
| | CAACAATGTATTCAGCAAGTCCTTCTTTGGGACCATCTTCCTC | |
| | TGTCCAGCCACTCTCTGGACTATCTAAGGCAAGCA | |
| | AGAAGGACTTGCTGAAT | 3154 |
| | ATTCAGCAAGTCCTTCT | 3155 |
| Non-polyposis | ACAGAGGAAGATGGTCCCAAAGAAGGACTTGCTGAATACATT | 3156 |
| colorectal cancer | GTTGAGTTTCTGAAGAAGAAGGCTGAGATGCTTGCAGACTAT | |
| Lys618Term | TTCTCTTTGGAAATTGATGAGGTGTGACAGCCATTCT | |
| AAG-TAG | AGAATGGCTGTCACACCTCATCAATTTCCAAAGAGAAATAGTC | 3157 |
| | TGCAAGCATCTCAGCCTTCTTCTTCAGAAACTCAACAATGTAT | |
| | TCAGCAAGTCCTTCTTTGGGACCATCTTCCTCTGT | |
| | TGAAGAAGAAGGCTGAG | 3158 |
| | CTCAGCCTACTTCTTCA | 3159 |
| Non-polyposis | CAGAGGAAGATGGTCCCAAAGAAGGACTTGCTGAATACATTG | 3160 |
| colorectal cancer | TTGAGTTTCTGAAGAAGAAGGCTGAGATGCTTGCAGACTATTT | |
| Lys618Thr | CTCTTTGGAAATTGATGAGGTGTGACAGCCATTCTT | |
| AAG-ACG | AAGAATGGCTGTCACACCTCATCAATTTCCAAAGAGAAATAGT | 3161 |
| | CTGCAAGCATCTCAGCCTTCTTCTTCAGAAACTCAACAATGTA | |
| | TTCAGCAAGTCCTTCTTTGGGACCATCTTCCTCTG | |
| | GAAGAAGAAGGCTGAGA | 3162 |
| | TCTCAGCCATCTTCTTC | 3163 |
| Non-polyposis | TACCCCTTCTGATTGACAACTATGTGCCCCCTTTGGAGGGAC | 3164 |
| colorectal cancer | TGCCTATCTTCATTCTTCGACTAGCCACTGAGGTCAGTGATCA | |
| Arg659Leu | AGCAGATACTAAGCATTTCGGTACATGCATGTGTGC | |
| CGA-CTA | GCACACATGCATGTACCGAAATGCTTAGTATCTGCTTGATCAC | 3165 |
| | TGACCTCAGTGGCTAGTCGAAGAATGAAGATAGGCAGTCCCT | |
| | CCAAAGGGGGCACATAGTTGTCAATCAGAAGGGGTA | |
| | CATTCTTCGACTAGCCA | 3166 |
| | TGGCTAGTCGAAGAATG | 3167 |
| Non-polyposis | TACCCCTTCTGATTGACAACTATGTGCCCCCTTTGGAGGGAC | 3168 |
| colorectal cancer | TGCCTATCTTCATTCTTCGACTAGCCACTGAGGTCAGTGATCA | |
| Arg659Pro | AGCAGATACTAAGCATTTCGGTACATGCATGTGTGC | |
| CGA-CCA | GCACACATGCATGTACCGAAATGCTTAGTATCTGCTTGATCAC | 3169 |
| | TGACCTCAGTGGCTAGTCGAAGAATGAAGATAGGCAGTCCCT | |
| | CCAAAGGGGGCACATAGTTGTCAATCAGAAGGGGTA | |
| | CATTCTTCGACTAGCCA | 3170 |
| | TGGCTAGTCGAAGAATG | 3171 |
| Non-polyposis | TTACCCCTTCTGATTGACAACTATGTGCCCCCTTTGGAGGGA | 3172 |
| colorectal cancer | CTGCCTATCTTCATTCTTCGACTAGCCACTGAGGTCAGTGATC | |
| Arg659Term | AAGCAGATACTAAGCATTTCGGTACATGCATGTGTG | |
| CGA-TGA | CACACATGCATGTACCGAAATGCTTAGTATCTGCTTGATCACT | 3173 |
| | GACCTCAGTGGCTAGTCGAAGAATGAAGATAGGCAGTCCCTC | |
| | CAAAGGGGGCACATAGTTGTCAATCAGAAGGGGTAA | |
| | TCATTCTTCGACTAGCC | 3174 |
| | GGCTAGTCGAAGAATGA | 3175 |
| Non-polyposis | TTGGACCAGGTGAATTGGGACGAAGAAAAGGAATGTTTTGAA | 3176 |
| colorectal cancer | AGCCTCAGTAAAGAATGCGCTATGTTCTATTCCATCCGGAAG | |
| Ala681Thr | CAGTACATATCTGAGGAGTCGACCCTCTCAGGCCAGC | |
| GCT-ACT | GCTGGCCTGAGAGGGTCGACTCCTCAGATATGTACTGCTTCC | 3177 |
| | GGATGGAATAGAACATAGCGCATTCTTTACTGAGGCTTTCAAA | |
| | ACATTCCTTTTCTTCGTCCCAATTCACCTGGTCCAA | |
| | AAGAATGCGCTATGTTC | 3178 |
| | GAACATAGCGCATTCTT | 3179 |
| Non-polyposis | AGGCTTATGACATCTAATGTGTTTTCCAGAGTGAAGTGCCTG | 3180 |
| colorectal cancer | GCTCCATTCCAAACTCCTGGAAGTGGACTGTGGAACACATTG | |
| Trp712Term | TCTATAAAGCCTTGCGCTCACACATTCTGCCTCCTAA | |
| TGG-TAG | TTAGGAGGCAGAATGTGTGAGCGCAAGGCTTTATAGACAATG | 3181 |
| | TGTTCCACAGTCCACTTCCAGGAGTTTGGAATGGAGCCAGGC | |
| | ACTTCACTCTGGAAAACACATTAGATGTCATAAGCCT | |
| | AAACTCCTGGAAGTGGA | 3182 |
| | TCCACTTCCAGGAGTTT | 3183 |
| Non-polyposis | ATGACATCTAATGTGTTTTCCAGAGTGAAGTGCCTGGCTCCAT | 3184 |
| colorectal cancer | TCCAAACTCCTGGAAGTGGACTGTGGAACACATTGTCTATAAA | |
| Trp714Term | GCCTTGCGCTCACACATTCTGCCTCCTAAACATTT | |
| TGG-TAG | AAATGTTTAGGAGGCAGAATGTGTGAGCGCAAGGCTTTATAG | 3185 |
| | ACAATGTGTTCCACAGTCCACTTCCAGGAGTTTGGAATGGAG | |
| | CCAGGCACTTCACTCTGGAAAACACATTAGATGTCAT | |
| | CTGGAAGTGGACTGTGG | 3186 |
| | CCACAGTCCACTTCCAG | 3187 |
| Non-polyposis | TGACATCTAATGTGTTTTCCAGAGTGAAGTGCCTGGCTCCATT | 3188 |
| colorectal cancer | CCAAACTCCTGGAAGTGGACTGTGGAACACATTGTCTATAAA | |
| Trp714Term | GCCTTGCGCTCACACATTCTGCCTCCTAAACATTTC | |
| TGG-TGA | GAAATGTTTAGGAGGCAGAATGTGTGAGCGCAAGGCTTTATA | 3189 |
| | GACAATGTGTTCCACAGTCCACTTCCAGGAGTTTGGAATGGA | |
| | GCCAGGCACTTCACTCTGGAAAACACATTAGATGTCA | |
| | TGGAAGTGGACTGTGGA | 3190 |
| | TCCACAGTCCACTTCCA | 3191 |
| Non-polyposis | ATCTAATGTGTTTTCCAGAGTGAAGTGCCTGGCTCCATTCCAA | 3192 |
| colorectal cancer | ACTCCTGGAAGTGGACTGTGGAACACATTGTCTATAAAGCCT | |
| Val716Met | TGCGCTCACACATTCTGCCTCCTAAACATTTCACAG | |
| GTG-ATG | CTGTGAAATGTTTAGGAGGCAGAATGTGTGAGCGCAAGGCTT | 3193 |
| | TATAGACAATGTGTTCCACAGTCCACTTCCAGGAGTTTGGAAT | |
| | GGAGCCAGGCACTTCACTCTGGAAAACACATTAGAT | |
| | AGTGGACTGTGGAACAC | 3194 |
| | GTGTTCCACAGTCCACT | 3195 |
| Non-polyposis | GAGTGAAGTGCCTGGCTCCATTCCAAACTCCTGGAAGTGGAC | 3196 |
| colorectal cancer | TGTGGAACACATTGTCTATAAAGCCTTGCGCTCACACATTCTG | |
| Tyr721Term | CCTCCTAAACATTTCACAGAAGATGGAAATATCCTG | |
| TAT-TAA | CAGGATATTTCCATCTTCTGTGAAATGTTTAGGAGGCAGAATG | 3197 |
| | TGTGAGCGCAAGGCTTTATAGACAATGTGTTCCACAGTCCAC | |
| | TTCCAGGAGTTTGGAATGGAGCCAGGCACTTCACTC | |
| | ATTGTCTATAAAGCCTT | 3198 |
| | AAGGCTTTATAGACAAT | 3199 |
| Non-polyposis | CTAAACATTTCACAGAAGATGGAAATATCCTGCAGCTTGCTAA | 3200 |
| colorectal cancer | CCTGCCTGATCTATACAAAGTCTTTGAGAGGTGTTAAATATGG | |
| Lys751Arg | TTATTTATGCACTGTGGGATGTGTTCTTCTTTCTC | |
| AAA-AGA | GAGAAAGAAGAACACATCCCACAGTGCATAAATAACCATATTT | 3201 |
| | AACACCTCTCAAAGACTTTGTATAGATCAGGCAGGTTAGCAA | |
| | GCTGCAGGATATTTCCATCTTCTGTGAAATGTTTAG | |
| | TCTATACAAAGTCTTTG | 3202 |
| | CAAAGACTTTGTATAGA | 3203 |
| Non-polyposis | ACAGAAGATGGAAATATCCTGCAGCTTGCTAACCTGCCTGAT | 3204 |
| colorectal cancer | CTATACAAAGTCTTTGAGAGGTGTTAAATATGGTTATTTATGC | |
| Arg755Trp | ACTGTGGGATGTGTTCTTCTTTCTCTGTATTCCGAT | |
| AGG-TGG | ATCGGAATACAGAGAAAGAAGAACACATCCCACAGTGCATAA | 3205 |
| | ATAACCATATTTAACACCTCTCAAAGACTTTGTATAGATCAGG | |
| | CAGGTTAGCAAGCTGCAGGATATTTCCATCTTCTGT | |
| | TCTTTGAGAGGTGTTAA | 3206 |
| | TTAACACCTCTCAAAGA | 3207 |

### EXAMPLE 19

### Human Mismatch Repair—MSH2

The human MSH2 gene is homologous to the bacterial mutS gene, which is involved in mismatch repair. Mutations in the MSH2 gene have been identified in a variety of cancers, including, for example, ovarian tumors, colorectal cancer, endometrial cancer, uterine cancer. The attached table discloses the correcting oligonucleotide base sequences for the MSH2 oligonucleotides of the invention.

TABLE 21
MSH2 Mutations and Genome-Correcting Oligos

| Clinical Phenotype & | | SEQ ID |
|---|---|---|
| Mutation | Correcting Oligos | NO: |
| Non polyposis | TTTTCCACAAAAGACATTTATCAGGACCTCAACCGGTTGTTGA | 3208 |
| colorectal cancer | AAGGCAAAAAGGGAGAGCAGATGAATAGTGCTGTATTGCCAG | |
| Gln252Term | AAATGGAGAATCAGGTACATGGATTATAAATGTGAA | |
| CAG-TAG | TTCACATTTATAATCCATGTACCTGATTCTCCATTTCTGGCAAT | 3209 |
| | ACAGCACTATTCATCTGCTCTCCCTTTTTGCCTTTCAACAACC | |
| | GGTTGAGGTCCTGATAAATGTCTTTTGTGGAAAA | |
| | AGGGAGAGCAGATGAAT | 3210 |
| | ATTCATCTGCTCTCCCT | 3211 |
| Non polyposis | TCATCACTGTCTGCGGTAATCAAGTTTTTAGAACTCTTATCAG | 3212 |
| colorectal cancer | ATGATTCCAACTTTGGACAGTTTGAACTGACTACTTTTGACTT | |
| Gln288Term | CAGCCAGTATATGAAATTGGATATTGCAGCAGTCA | |
| CAG-TAG | TGACTGCTGCAATATCCAATTTCATATACTGGCTGAAGTCAAA | 3213 |
| | AGTAGTCAGTTCAAACTGTCCAAAGTTGGAATCATCTGATAAG | |
| | AGTTCTAAAAACTTGATTACCGCAGACAGTGATGA | |
| | ACTTTGGACAGTTTGAA | 3214 |
| | TTCAAACTGTCCAAAGT | 3215 |
| Non polyposis | AACTTTGGACAGTTTGAACTGACTACTTTTGACTTCAGCCAGT | 3216 |
| colorectal cancer | ATATGAAATTGGATATTGCAGCAGTCAGAGCCCTTAACCTTTT | |
| Ala305Thr | TCAGGTAAAAAAAAAAAAAAAAAAAAAAAAAAAGG | |
| GCA-ACA | CCTTTTTTTTTTTTTTTTTTTTTTTTTTTACCTGAAAAAGGTTAA | 3217 |
| | GGGCTCTGACTGCTGCAATATCCAATTTCATATACTGGCTGAA | |
| | GTCAAAAGTAGTCAGTTCAAACTGTCCAAAGTT | |
| | TGGATATTGCAGCAGTC | 3218 |
| | GACTGCTGCAATATCCA | 3219 |
| Non polyposis | AGCTTGCCATTCTTTCTATTTTATTTTTTGTTTACTAGGGTTCT | 3220 |
| colorectal cancer | GTTGAAGATACCACTGGCTCTCAGTCTCTGGCTGCCTTGCTG | |
| Gly322Asp | AATAAGTGTAAAACCCCTCAAGGACAAAGACTTGT | |
| GGC-GAC | ACAAGTCTTTGTCCTTGAGGGGTTTTACACTTATTCAGCAAGG | 3221 |
| | CAGCCAGAGACTGAGAGCCAGTGGTATCTTCAACAGAACCCT | |
| | AGTAAACAAAAAATAAAATAGAAAGAATGGCAAGCT | |
| | TACCACTGGCTCTCAGT | 3222 |
| | ACTGAGAGCCAGTGGTA | 3223 |
| Non polyposis | TTGCCATTCTTTCTATTTTATTTTTTGTTTACTAGGGTTCTGTT | 3224 |
| colorectal cancer | GAAGATACCACTGGCTCTCAGTCTCTGGCTGCCTTGCTGAAT | |
| Ser323Cys | AAGTGTAAAACCCCTCAAGGACAAAGACTTGTTAA | |
| TCT-TGT | TTAACAAGTCTTTGTCCTTGAGGGGTTTTACACTTATTCAGCA | 3225 |
| | AGGCAGCCAGAGACTGAGAGCCAGTGGTATCTTCAACAGAAC | |
| | CCTAGTAAACAAAAAATAAAATAGAAAGAATGGCAA | |
| | CACTGGCTCTCAGTCTC | 3226 |
| | GAGACTGAGAGCCAGTG | 3227 |
| Non polyposis | GTGGAAGCTTTTGTAGAAGATGCAGAATTGAGGCAGACTTTA | 3228 |
| colorectal cancer | CAAGAAGATTTACTTCGTCGATTCCCAGATCTTAACCGACTTG | |
| Arg383Term | CCAAGAAGTTTCAAAGACAAGCAGCAAACTTACAAG | |
| CGA-TGA | CTTGTAAGTTTGCTGCTTGTCTTTGAAACTTCTTGGCAAGTCG | 3229 |
| | GTTAAGATCTGGGAATCGACGAAGTAAATCTTCTTGTAAAGTC | |
| | TGCCTCAATTCTGCATCTTCTACAAAAGCTTCCAC | |
| | TACTTCGTCGATTCCCA | 3230 |
| | TGGGAATCGACGAAGTA | 3231 |
| Non polyposis | CAAGAAGATTTACTTCGTCGATTCCCAGATCTTAACCGACTTG | 3232 |
| colorectal cancer | CCAAGAAGTTTCAAAGACAAGCAGCAAACTTACAAGATTGTTA | |
| Gln397Term | CCGACTCTATCAGGGTATAAATCAACTACCTAATG | |
| CAA-TAA | CATTAGGTAGTTGATTTATACCCTGATAGAGTCGGTAACAATC | 3233 |
| | TTGTAAGTTTGCTGCTTGTCTTTGAAACTTCTTGGCAAGTCGG | |
| | TTAAGATCTGGGAATCGACGAAGTAAATCTTCTTG | |
| | TTCAAAGACAAGCAGCA | 3234 |
| | TGCTGCTTGTCTTTGAA | 3235 |
| Non polyposis | GATCTTAACCGACTTGCCAAGAAGTTTCAAAGACAAGCAGCA | 3236 |
| colorectal cancer | AACTTACAAGATTGTTACCGACTCTATCAGGGTATAAATCAAC | |
| Arg406Term | TACCTAATGTTATACAGGCTCTGGAAAAACATGAAG | |
| CGA-TGA | CTTCATGTTTTTCCAGAGCCTGTATAACATTAGGTAGTTGATTT | 3237 |
| | ATACCCTGATAGAGTCGGTAACAATCTTGTAAGTTTGCTGCTT | |
| | GTCTTTGAAACTTCTTGGCAAGTCGGTTAAGATC | |
| | ATTGTTACCGACTCTAT | 3238 |
| | ATAGAGTCGGTAACAAT | 3239 |
| Non polyposis | GCAAACTTACAAGATTGTTACCGACTCTATCAGGGTATAAATC | 3240 |
| colorectal cancer | AACTACCTAATGTTATACAGGCTCTGGAAAAACATGAAGGTAA | |
| Gln419Term | CAAGTGATTTTGTTTTTTTGTTTTCCTTCAACTCA | |
| CAG-TAG | TGAGTTGAAGGAAAACAAAAAAACAAAATCACTTGTTACCTTC | 3241 |
| | ATGTTTTTCCAGAGCCTGTATAACATTAGGTAGTTGATTTATAC | |
| | CCTGATAGAGTCGGTAACAATCTTGTAAGTTTGC | |
| | ATGTTATACAGGCTCTG | 3242 |
| | CAGAGCCTGTATAACAT | 3243 |
| Non polyposis | TATTCTGTAAAATGAGATCTTTTTATTTGTTTGTTTTACTACTTT | 3244 |
| colorectal cancer | CTTTTAGGAAAACACCAGAAATTATTGTTGGCAGTTTTTGTGA | |
| Gln429Term | CTCCTCTTACTGATCTTCGTTCTGACTTCTCCA | |
| CAG-TAG | TGGAGAAGTCAGAACGAAGATCAGTAAGAGGAGTCACAAAAA | 3245 |
| | CTGCCAACAATAATTTCTGGTGTTTTCCTAAAAGAAAGTAGTA | |
| | AAACAAACAAATAAAAAGATCTCATTTTACAGAATA | |
| | GAAAACACCAGAAATTA | 3246 |
| | TAATTTCTGGTGTTTTC | 3247 |
| Non polyposis | CTCCTCTTACTGATCTTCGTTCTGACTTCTCCAAGTTTCAGGA | 3248 |
| colorectal cancer | AATGATAGAAACAACTTTAGATATGGATCAGGTATGCAATATA | |
| Leu458Term | CTTTTTAATTTAAGCAGTAGTTATTTTTAAAAAGC | |
| TTA-TGA | GCTTTTTAAAAATAACTACTGCTTAAATTAAAAAGTATATTGCA | 3249 |
| | TACCTGATCCATATCTAAAGTTGTTTCTATCATTTCCTGAAACT | |
| | TGGAGAAGTCAGAACGAAGATCAGTAAGAGGAG | |
| | AACAACTTTAGATATGG | 3250 |
| | CCATATCTAAAGTTGTT | 3251 |
| Non polyposis | TTTCTTCTTGATTATCAAGGCTTGGACCCTGGCAAACAGATTA | 3252 |
| colorectal cancer | AACTGGATTCCAGTGCACAGTTTGGATATTACTTTCGTGTAAC | |
| Gln518Term | CTGTAAGGAAGAAAAAGTCCTTCGTAACAATAAAA | |
| CAG-TAG | TTTTATTGTTACGAAGGACTTTTTCTTCCTTACAGGTTACACGA | 3253 |
| | AAGTAATATCCAAACTGTGCACTGGAATCCAGTTTAATCTGTT | |
| | TGCCAGGGTCCAAGCCTTGATAATCAAGAAGAAA | |
| | CCAGTGCACAGTTTGGA | 3254 |
| | TCCAAACTGTGCACTGG | 3255 |
| Non polyposis | GCTTGGACCCTGGCAAACAGATTAAACTGGATTCCAGTGCAC | 3256 |
| colorectal cancer | AGTTTGGATATTACTTTCGTGTAACCTGTAAGGAAGAAAAAGT | |
| Arg524Pro | CCTTCGTAACAATAAAAACTTTAGTACTGTAGATAT | |
| CGT-CCT | ATATCTACAGTACTAAAGTTTTTATTGTTACGAAGGACTTTTTC | 3257 |
| | TTCCTTACAGGTTACACGAAAGTAATATCCAAACTGTGCACTG | |
| | GAATCCAGTTTAATCTGTTTGCCAGGGTCCAAGC | |
| | TTACTTTCGTGTAACCT | 3258 |
| | AGGTTACACGAAAGTAA | 3259 |
| Non polyposis | TTAATATTTTTAATAAAACTGTTATTTCGATTTGCAGCAAATTG | 3260 |
| colorectal cancer | ACTTCTTTAAATGAAGAGTATACCAAAAATAAAACAGAATATG | |
| Glu562Val | AAGAAGCCCAGGATGCCATTGTTAAAGAAATTGT | |
| GAG-GTG | ACAATTTCTTTAACAATGGCATCCTGGGCTTCTTCATATTCTGT | 3261 |
| | TTTATTTTTGGTATACTCTTCATTTAAAGAAGTCAATTTGCTGC | |
| | AAATCGAAATAACAGTTTTATTAAAAATATTAA | |
| | AAATGAAGAGTATACCA | 3262 |
| | TGGTATACTCTTCATTT | 3263 |
| Glioma | AATGAAGAGTATACCAAAAATAAAACAGAATATGAAGAAGCCC | 3264 |
| Glu580Term | AGGATGCCATTGTTAAAGAAATTGTCAATATTTCTTCAGGTAA | |
| GAA-TAA | ACTTAATAGAACTAATAATGTTCTGAATGTCACCT | |
| | AGGTGACATTCAGAACATTATTAGTTCTATTAAGTTTACCTGAA | 3265 |
| | GAAATATTGACAATTTCTTTAACAATGGCATCCTGGGCTTCTT | |
| | CATATTCTGTTTTATTTTTGGTATACTCTTCATT | |
| | TTGTTAAAGAAATTGTC | 3266 |
| | GACAATTTCTTTAACAA | 3267 |
| Non polyposis | TGTTTTTATTTTTATACAGGCTATGTAGAACCAATGCAGACACT | 3268 |
| colorectal cancer | CAATGATGTGTTAGCTCAGCTAGATGCTGTTGTCAGCTTTGCT | |
| Gln601Term | CACGTGTCAAATGGAGCACCTGTTCCATATGTAC | |
| GAG-TAG | GTACATATGGAACAGGTGCTCCATTTGACACGTGAGCAAAGC | 3269 |
| | TGACAACAGCATCTAGCTGAGCTAACACATCATTGAGTGTCT | |
| | GCATTGGTTCTACATAGCCTGTATAAAAATAAAAACA | |
| | TGTTAGCTCAGCTAGAT | 3270 |
| | ATCTAGCTGAGCTAACA | 3271 |
| Non polyposis | AGCTCAGCTAGATGCTGTTGTCAGCTTTGCTCACGTGTCAAAT | 3272 |
| colorectal cancer | GGAGCACCTGTTCCATATGTACGACCAGCCATTTTGGAGAAA | |
| Tyr619Term | GGACAAGGAAGAATTATATTAAAAGCATCCAGGCAT | |
| TAT-TAG | ATGCCTGGATGCTTTTAATATAATTCTTCCTTGTCCTTTCTCCA | 3273 |
| | AAATGGCTGGTCGTACATATGGAACAGGTGCTCCATTTGACA | |
| | CGTGAGCAAAGCTGACAACAGCATCTAGCTGAGCT | |
| | GTTCCATATGTACGACC | 3274 |
| | GGTCGTACATATGGAAC | 3275 |
| Non polyposis | CAGCTAGATGCTGTTGTCAGCTTTGCTCACGTGTCAAATGGA | 3276 |
| colorectal cancer | GCACCTGTTCCATATGTACGACCAGCCATTTTGGAGAAAGGA | |
| Arg621Term | CAAGGAAGAATTATATTAAAAGCATCCAGGCATGCTT | |
| CGA-TGA | AAGCATGCCTGGATGCTTTTAATATAATTCTTCCTTGTCCTTTC | 3277 |
| | TCCAAAATGGCTGGTCGTACATATGGAACAGGTGCTCCATTT | |
| | GACACGTGAGCAAAGCTGACAACAGCATCTAGCTG | |
| | CATATGTACGACCAGCC | 3278 |
| | GGCTGGTCGTACATATG | 3279 |
| Non polyposis | TAGATGCTGTTGTCAGCTTTGCTCACGTGTCAAATGGAGCAC | 3280 |
| colorectal cancer | CTGTTCCATATGTACGACCAGCCATTTTGGAGAAAGGACAAG | |
| Pro622Leu | GAAGAATTATATTAAAAGCATCCAGGCATGCTTGTGT | |
| CCA-CTA | ACACAAGCATGCCTGGATGCTTTTAATATAATTCTTCCTTGTC | 3281 |
| | CTTTCTCCAAAATGGCTGGTCGTACATATGGAACAGGTGCTC | |
| | CATTTGACACGTGAGCAAAGCTGACAACAGCATCTA | |
| | TGTACGACCAGCCATTT | 3282 |
| | AAATGGCTGGTCGTACA | 3283 |
| Non polyposis | CCTGTTCCATATGTACGACCAGCCATTTTGGAGAAAGGACAA | 3284 |
| colorectal cancer | GGAAGAATTATATTAAAAGCATCCAGGCATGCTTGTGTTGAAG | |
| Ala636Pro | TTCAAGATGAAATTGCATTTATTCCTAATGACGTAT | |
| GCA-CCA | ATACGTCATTAGGAATAAATGCAATTTCATCTTGAACTTCAACA | 3285 |
| | CAAGCATGCCTGGATGCTTTTAATATAATTCTTCCTTGTCCTTT | |
| | CTCCAAAATGGCTGGTCGTACATATGGAACAGG | |
| | TATTAAAAGCATCCAGG | 3286 |
| | CCTGGATGCTTTTAATA | 3287 |
| Non polyposis | ATGTACGACCAGCCATTTTGGAGAAAGGACAAGGAAGAATTA | 3288 |
| colorectal cancer | TATTAAAAGCATCCAGGCATGCTTGTGTTGAAGTTCAAGATGA | |
| His639Arg | AATTGCATTTATTCCTAATGACGTATACTTTGAAAA | |
| CAT-CGT | TTTTCAAAGTATACGTCATTAGGAATAAATGCAATTTCATCTTG | 3289 |
| | AACTTCAACACAAGCATGCCTGGATGCTTTTAATATAATTCTT | |
| | CCTTGTCCTTTCTCCAAAATGGCTGGTCGTACAT | |
| | ATCCAGGCATGCTTGTG | 3290 |
| | CACAAGCATGCCTGGAT | 3291 |
| Non polyposis | TATGTACGACCAGCCATTTTGGAGAAAGGACAAGGAAGAATT | 3292 |
| colorectal cancer | ATATTAAAAGCATCCAGGCATGCTTGTGTTGAAGTTCAAGATG | |
| His639Tyr | AAATTGCATTTATTCCTAATGACGTATACTTTGAAA | |
| CAT-TAT | TTTCAAAGTATACGTCATTAGGAATAAATGCAATTTCATCTTGA | 3293 |
| | ACTTCAACACAAGCATGCCTGGATGCTTTTAATATAATTCTTC | |
| | CTTGTCCTTTCTCCAAAATGGCTGGTCGTACATA | |
| | CATCCAGGCATGCTTGT | 3294 |
| | ACAAGCATGCCTGGATG | 3295 |
| Non polyposis | AAAGGACAAGGAAGAATTATATTAAAAGCATCCAGGCATGCTT | 3296 |
| colorectal cancer | GTGTTGAAGTTCAAGATGAAATTGCATTTATTCCTAATGACGT | |
| Glu647Lys | ATACTTTGAAAAAGATAAACAGATGTTCCACATCA | |
| GAA-AAA | TGATGTGGAACATCTGTTTATCTTTTTCAAAGTATACGTCATTA | 3297 |
| | GGAATAAATGCAATTTCATCTTGAACTTCAACACAAGCATGCC | |
| | TGGATGCTTTTAATATAATTCTTCCTTGTCCTTT | |
| | TTCAAGATGAAATTGCA | 3298 |
| | TGCAATTTCATCTTGAA | 3299 |
| Non polyposis | ATCCAGGCATGCTTGTGTTGAAGTTCAAGATGAAATTGCATTT | 3300 |
| colorectal cancer | ATTCCTAATGACGTATACTTTGAAAAAGATAAACAGATGTTCC | |
| Tyr656Term | ACATCATTACTGGTAAAAAACCTGGTTTTTGGGCT | |
| TAC-TAG | AGCCCAAAAACCAGGTTTTTTACCAGTAATGATGTGGAACATC | 3301 |
| | TGTTTATCTTTTTCAAAGTATACGTCATTAGGAATAAATGCAAT | |
| | TTCATCTTGAACTTCAACACAAGCATGCCTGGAT | |
| | GACGTATACTTTGAAAA | 3302 |
| | TTTTCAAAGTATACGTC | 3303 |
| Non polyposis | GAAAGAAGTTTAAAATCTTGCTTTCTGATATAATTTGTTTTGTA | 3304 |
| colorectal cancer | GGCCCCAATATGGGAGGTAAATCAACATATATTCGACAAACT | |
| Gly674Asp | GGGGTGATAGTACTCATGGCCCAAATTGGGTGTTT | |
| GGT-GAT | AAACACCCAATTTGGGCCATGAGTACTATCACCCCAGTTTGTC | 3305 |
| | GAATATATGTTGATTTACCTCCCATATTGGGGCCTACAAAACA | |
| | AATTATATCAGAAAGCAAGATTTTAAACTTCTTTC | |
| | TATGGGAGGTAAATCAA | 3306 |
| | TTGATTTACCTCCCATA | 3307 |
| Non polyposis | TTGCTTTCTGATATAATTTGTTTTGTAGGCCCCAATATGGGAG | 3308 |
| colorectal cancer | GTAAATCAACATATATTCGACAAACTGGGGTGATAGTACTCAT | |
| Arg680Term | GGCCCAAATTGGGTGTTTTGTGCCATGTGAGTCAG | |
| CGA-TGA | CTGACTCACATGGCACAAAACACCCAATTTGGGCCATGAGTA | 3309 |
| | CTATCACCCCAGTTTGTCGAATATATGTTGATTTACCTCCCAT | |
| | ATTGGGGCCTACAAAACAAATTATATCAGAAAGCAA | |
| | CATATATTCGACAAACT | 3310 |
| | AGTTTGTCGAATATATG | 3311 |
| Non polyposis | ATGGGAGGTAAATCAACATATATTCGACAAACTGGGGTGATA | 3312 |
| colorectal cancer | GTACTCATGGCCCAAATTGGGTGTTTTGTGCCATGTGAGTCA | |
| Gly692Arg | GCAGAAGTGTCCATTGTGGACTGCATCTTAGCCCGAG | |
| GGG-CGG | CTCGGGCTAAGATGCAGTCCACAATGGACACTTCTGCTGACT | 3313 |
| | CACATGGCACAAAACACCCAATTTGGGCCATGAGTACTATCA | |
| | CCCCAGTTTGTCGAATATATGTTGATTTACCTCCCAT | |
| | CCCAAATTGGGTGTTTT | 3314 |
| | AAAACACCCAATTTGGG | 3315 |
| Non polyposis | ACATATATTCGACAAACTGGGGTGATAGTACTCATGGCCCAA | 3316 |
| colorectal cancer | ATTGGGTGTTTTGTGCCATGTGAGTCAGCAGAAGTGTCCATT | |
| Cys697Arg | GTGGACTGCATCTTAGCCCGAGTAGGGGCTGGTGACA | |
| TGT-CGT | TGTCACCAGCCCCTACTCGGGCTAAGATGCAGTCCACAATGG | 3317 |
| | ACACTTCTGCTGACTCACATGGCACAAAACACCCAATTTGGG | |
| | CCATGAGTACTATCACCCCAGTTTGTCGAATATATGT | |
| | TTGTGCCATGTGAGTCA | 3318 |
| | TGACTCACATGGCACAA | 3319 |
| Non polyposis | CATATATTCGACAAACTGGGGTGATAGTACTCATGGCCCAAAT | 3320 |
| colorectal cancer | TGGGTGTTTTGTGCCATGTGAGTCAGCAGAAGTGTCCATTGT | |
| Cys697Phe | GGACTGCATCTTAGCCCGAGTAGGGGCTGGTGACAG | |
| TGT-TTT | CTGTCACCAGCCCCTACTCGGGCTAAGATGCAGTCCACAATG | 3321 |
| | GACACTTCTGCTGACTCACATGGCACAAAACACCCAATTTGG | |
| | GCCATGAGTACTATCACCCCAGTTTGTCGAATATATG | |
| | TGTGCCATGTGAGTCAG | 3322 |
| | CTGACTCACATGGCACA | 3323 |
| Non polyposis | GAGTCAGCAGAAGTGTCCATTGTGGACTGCATCTTAGCCCGA | 3324 |
| colorectal cancer | GTAGGGGCTGGTGACAGTCAATTGAAAGGAGTCTCCACGTTC | |
| Gln718Term | ATGGCTGAAATGTTGGAAACTGCTTCTATCCTCAGGT | |
| CAA-TAA | ACCTGAGGATAGAAGCAGTTTCCAACATTTCAGCCATGAACG | 3325 |
| | TGGAGACTCCTTTCAATTGACTGTCACCAGCCCCTACTCGGG | |
| | CTAAGATGCAGTCCACAATGGACACTTCTGCTGACTC | |
| | GTGACAGTCAATTGAAA | 3326 |
| | TTTCAATTGACTGTCAC | 3327 |
| Non polyposis | CCAATCAGATACCAACTGTTAATAATCTACATGTCACAGCACT | 3328 |
| colorectal cancer | CACCACTGAAGAGACCTTAACTATGCTTTATCAGGTGAAGAAA | |
| Leu811Term | GGTATGTACTATTGGAGTACTCTAAATTCAGAACT | |
| TTA-TGA | AGTTCTGAATTTAGAGTACTCCAATAGTACATACCTTTCTTCAC | 3329 |
| | CTGATAAAGCATAGTTAAGGTCTCTTCAGTGGTGAGTGCTGT | |
| | GACATGTAGATTATTAACAGTTGGTATCTGATTGG | |
| | AGAGACCTTAACTATGC | 3330 |
| | GCATAGTTAAGGTCTCT | 3331 |
| Non polyposis | TTCCCCAAATTTCTTATAGGTGTCTGTGATCAAAGTTTTGGGA | 3332 |
| colorectal cancer | TTCATGTTGCAGAGCTTGCTAATTTCCCTAAGCATGTAATAGA | |
| Ala834Thr | GTGTGCTAAACAGAAAGCCCTGGAACTTGAGGAGT | |
| GCT-ACT | ACTCCTCAAGTTCCAGGGCTTTCTGTTTAGCACACTCTATTAC | 3333 |
| | ATGCTTAGGGAAATTAGCAAGCTCTGCAACATGAATCCCAAAA | |
| | CTTTGATCACAGACACCTATAAGAAATTTGGGGAA | |
| | CAGAGCTTGCTAATTTC | 3334 |
| | GAAATTAGCAAGCTCTG | 3335 |
| Non polyposis | ATAGAGTGTGCTAAACAGAAAGCCCTGGAACTTGAGGAGTTT | 3336 |
| colorectal cancer | CAGTATATTGGAGAATCGCAAGGATATGATATCATGGAACCA | |
| Gln861Term | GCAGCAAAGAAGTGCTATCTGGAAAGAGAGGTTTGTC | |
| CAA-TAA | GACAAACCTCTCTTTCCAGATAGCACTTCTTTGCTGCTGGTTC | 3337 |
| | CATGATATCATATCCTTGCGATTCTCCAATATACTGAAACTCC | |
| | TCAAGTTCCAGGGCTTTCTGTTTAGCACACTCTAT | |
| | GAGAATCGCAAGGATAT | 3338 |
| | ATATCCTTGCGATTCTC | 3339 |
| Non polyposis | AGGAGTTCCTGTCCAAGGTGAAACAAATGCCCTTTACTGAAAT | 3340 |
| colorectal cancer | GTCAGAAGAAAACATCACAATAAAGTTAAAACAGCTAAAAGCT | |
| Thr905Arg | GAAGTAATAGCAAAGAATAATAGCTTTGTAAATGA | |
| ACA-AGA | TCATTTACAAAGCTATTATTCTTTGCTATTACTTCAGCTTTTAG | 3341 |
| | CTGTTTTAACTTTATTGTGATGTTTTCTTCTGACATTTCAGTAA | |
| | AGGGCATTTGTTTCACCTTGGACAGGAACTCCT | |
| | AAACATCACAATAAAGT | 3342 |
| | ACTTTATTGTGATGTTT | 3343 |

### EXAMPLE 20

### Human Mismatch Repair—MSH6

The human MSH6 gene is homologous to the bacterial mutS gene, which is involved in mismatch repair. Mutations in the MSH6 gene have been identified in a variety of cancers, including particularly hereditary nonpolyposis colorectal cancer. The attached table discloses the correcting oligonucleotide base sequences for the MSH6 oligonucleotides of the invention.

TABLE 22
MSH6 Mutations and Genome-Correcting Oligos

| Clinical Phenotype & | | SEQ ID |
|---|---|---|
| Mutation | Correcting Oligos | NO: |
| Non-polyposis | GGAAATCAGTCCGTGTTCATGTACAGTTTTTTGATGACAGCCC | 3344 |
| colorectal cancer | AACAAGGGGCTGGGTTAGCAAAAGGCTTTTAAAGCCATATAC | |
| Ser144Ile | AGGTAAGAGTCACTACTGCCATGTGTGTGTGTTTGT | |
| AGC-ATC | ACAAACACACACACATGGCAGTAGTGACTCTTACCTGTATATG | 3345 |
| | GCTTTAAAAGCCTTTTGCTAACCCAGCCCCTTGTTGGGCTGT | |
| | CATCAAAAAACTGTACATGAACACGGACTGATTTCC | |
| | CTGGGTTAGCAAAAGGC | 3346 |
| | GCCTTTTGCTAACCCAG | 3347 |
| Endometrial cancer | CGTGAGCCTCTGCACCCGGCCCTTATTGTTTATAAATACATTT | 3348 |
| Ser156Term | CTTTCTAGGTTCAAAATCAAAGGAAGCCCAGAAGGGAGGTCA | |
| TCA-TGA | TTTTTACAGTGCAAAGCCTGAAATACTGAGAGCAAT | |
| | ATTGCTCTCAGTATTTCAGGCTTTGCACTGTAAAAATGACCTC | 3349 |
| | CCTTCTGGGCTTCCTTTGATTTTGAACCTAGAAAGAAATGTAT | |
| | TTATAAACAATAAGGGCCGGGTGCAGAGGCTCACG | |
| | TTCAAAATCAAAGGAAG | 3350 |
| | CTTCCTTTGATTTTGAA | 3351 |
| Early onset colorectal | TTCCAAATTTIGATTTGTTTTTAAATACTCTTTCCTTGCCTGGC | 3352 |
| cancer | AGGTAGGCACAACTTACGTAACAGATAAGAGTGAAGAAGATA | |
| Tyr214Term | ATGAAATTGAGAGTGAAGAGGAAGTACAGCCTAAG | |
| TAC-TAG | CTTAGGCTGTACTTCCTCTTCACTCTCAATTTCATTATCTTCTT | 3353 |
| | CACTCTTATCTGTTACGTAAGTTGTGCCTACCTGCCAGGCAA | |
| | GGAAAGAGTATTTAAAAACAAATCAAAATTTGGAA | |
| | ACAACTTACGTAACAGA | 3354 |
| | TCTGTTACGTAAGTTGT | 3355 |
| Endometrial cancer | GAAGAGGAAGTACAGCCTAAGACACAAGGATCTAGGCGAAGT | 3356 |
| Arg248Term | AGCCGCCAAATAAAAAAACGAAGGGTCATATCAGATTCTGAG | |
| CGA-TGA | AGTGACATTGGTGGCTCTGATGTGGAATTTAAGCCAG | |
| | CTGGCTTAAATTCCACATCAGAGCCACCAATGTCACTCTCAGA | 3357 |
| | ATCTGATATGACCCTTCGTTTTTTTATTTGGCGGCTACTTCGC | |
| | CTAGATCCTTGTGTCTTAGGCTGTACTTCCTCTTC | |
| | TAAAAAAAGGAAGGGTC | 3358 |
| | GACCCTTCGTTTTTTTA | 3359 |
| Colorectal cancer | TTAAGCCAGACACTAAGGAGGAAGGAAGCAGTGATGAAATAA | 3360 |
| Ser285Ile | GCAGTGGAGTGGGGGATAGTGAGAGTGAAGGCCTGAACAGC | |
| AGT-ATT | CCTGTCAAAGTTGCTCGAAAGCGGAAGAGAATGGTGAC | |
| | GTCACCATTCTCTTCCGCTTTCGAGCAACTTTGACAGGGCTG | 3361 |
| | TTCAGGCCTTCACTCTCACTATCCCCCACTCCACTGCTTATTT | |
| | CATCACTGCTTCCTTCCTCCTTAGTGTCTGGCTTAA | |
| | GGGGGATAGTGAGAGTG | 3362 |
| | CACTCTCACTATCCCCC | 3363 |
| Colorectal cancer | GAGGAAGATTCTTCTGGCCATACTCGTGCATATGGTGTGTGC | 3364 |
| Gly566Arg | TTTGTTGATACTTCACTGGGAAAGTTTTTCATAGGTCAGTTTTC | |
| GGA-AGA | AGATGATCGCCATTGTTCGAGATTTAGGACTCTAG | |
| | CTAGAGTCCTAAATCTCGAACAATGGCGATCATCTGAAAACTG | 3365 |
| | ACCTATGAAAAACTTTCCCAGTGAAGTATCAACAAAGCACACA | |
| | CCATATGCACGAGTATGGCCAGAAGAATCTTCCTC | |
| | CTTCACTGGGAAAGTTT | 3366 |
| | AAACTTTCCCAGTGAAG | 3367 |
| Non-polyposis | GAATTGGCCCTCTCTGCTCTAGGTGGTTGTGTCTTCTACCTCA | 3368 |
| colorectal cancer | AAAAATGCCTTATTGATCAGGAGCTTTTATCAATGGCTAATTTT | |
| Gln698Glu | GAAGAATATATTCCCTTGGATTCTGACACAGTCA | |
| CAG-GAG | TGACTGTGTCAGAATCCAAGGGAATATATTCTTCAAAATTAGC | 3369 |
| | CATTGATAAAAGCTCCTGATCAATAAGGCATTTTTTGAGGTAG | |
| | AAGACACAACCACCTAGAGCAGAGAGGGCCAATTC | |
| | TTATTGATCAGGAGCTT | 3370 |
| | AAGCTCCTGATCAATAA | 3371 |
| Endometrial cancer | CCCTTGGATTCTGACACAGTCAGCACTACAAGATCTGGTGCT | 3372 |
| Gln731Term | ATCTTCACCAAAGCCTATCAACGAATGGTGCTAGATGCAGTG | |
| CAA-TAA | ACATTAAACAACTTGGAGATTTTTCTGAATGGAACAA | |
| | TTGTTCCATTCAGAAAAATCTCCAAGTTGTTTAATGTCACTGC | 3373 |
| | ATCTAGCACCATTCGTTGATAGGCTTTGGTGAAGATAGCACC | |
| | AGATCTTGTAGTGCTGACTGTGTCAGAATCCAAGGG | |
| | AAGCCTATCAACGAATG | 3374 |
| | CATTCGTTGATAGGCTT | 3375 |
| Colorectal cancer | GCCCCACTCTGTAACCATTATGCTATTAATGATCGTCTAGATG | 3376 |
| Val800Leu | CCATAGAAGACCTCATGGTTGTGCCTGACAAAATCTCCGAAG | |
| GTT-CTT | TTGTAGAGCTTCTAAAGTAAGCTTCCAGATCTTGAGA | |
| | TCTCAAGATCTGGAAGCTTCTTTAGAAGCTCTACAACTTCGGA | 3377 |
| | GATTTTGTCAGGCACAACCATGAGGTCTTCTATGGCATCTAGA | |
| | CGATCATTAATAGCATAATGGTTACAGAGTGGGGC | |
| | ACCTCATGGTTGTGCCT | 3378 |
| | AGGCACAACCATGAGGT | 3379 |
| Colorectal cancer | GTAACCATTATGCTATTAATGATCGTCTAGATGCCATAGAAGA | 3380 |
| Asp803Gly | CCTCATGGTTGTGCCTGACAAAATCTCCGAAGTTGTAGAGCT | |
| GAC-GGC | TCTAAAGAAGCTTCCAGATCTTGAGAGGCTACTCAG | |
| | CTGAGTAGCCTCTCAAGATCTGGAAGCTTCTTTAGAAGCTCTA | 3381 |
| | CAACTTCGGAGATTTTGTCAGGCACAACCATGAGGTCTTCTAT | |
| | GGCATCTAGACGATCATTAATAGCATTAATGGTTAC | |
| | TGTGCCTGACAAAATCT | 3382 |
| | AGATTTTGTCAGGCACA | 3383 |
| Non-polyposis | CTCCCCTGAAGAGTCAGAACCACCCAGACAGCAGGGCTATAA | 3384 |
| colorectal cancer | TGTATGAAGAAACTACATACAGCAAGAAGAAGATTATTGATTT | |
| Tyr850Cys | TCTTTCTGCTCTGGAAGGATTCAAAGTAATGTGTAA | |
| TAC-TGC | TTACACATTACTTTGAATCCTTCCAGAGCAGAAAGAAAATCAA | 3385 |
| | TAATCTTCTTCTTGCTGTATGTAGTTTCTTCATACATTATAGCC | |
| | CTGCTGTCTGGGTGGTTCTGACTCTTCAGGGGAG | |
| | AACTACATACAGCAAGA | 3386 |
| | TCTTGCTGTATGTAGTT | 3387 |
| Colorectal cancer | TATAGTCGAGGGGGTGATGGTCCTATGTGTCGCCCAGTAATT | 3388 |
| Pro1087Thr | CTGTTGCCGGAAGATACCCCCCCCTTCTTAGAGCTTAAAGGA | |
| CCC-ACC | TCACGCCATCCTTGCATTACGAAGACTTTTTTTGGAG | |
| | CTCCAAAAAAAGTCTTCGTAATGCAAGGATGGCGTGATCCTTT | 3389 |
| | AAGCTCTAAGAAGGGGGGGGTATCTTCCGGCAACAGAATTAC | |
| | TGGGCGACACATAGGACCATCACCCCCTCGACTATA | |
| | AAGATACCCCCCCCTTC | 3390 |
| | GAAGGGGGGGGTATCTT | 3391 |
| Non-polyposis | ACTATAAAATGTCGTACATTATTTTCAACTCACTACCATTCATT | 3392 |
| colorectal cancer | AGTAGAAGATTATTCTCAAAATGTTGCTGTGCGCCTAGGACAT | |
| Gln1258Term | ATGGTATGTGCAAATTGTTTTTTTCCACAAATTC | |
| CAA-TAA | GAATTTGTGGAAAAAAACAATTTGCACATACCATATGTCCTAG | 3393 |
| | GCGCACAGCAACATTTTGAGAATAATCTTCTACTAATGAATGG | |
| | TAGTGAGTTGAAAATAATGTACGACATTTTATAGT | |
| | ATTATTCTCAAAATGTT | 3394 |
| | AACATTTTGAGAATAAT | 3395 |

### EXAMPLE 21

### Hyperlipidemia APOE

Hyperlipidemia is the abnormal elevation of plasma cholesterol and/or triglyceride levels and it is one of the most common diseases. The human apolipoprotein E protein is involved in the transport of endogenous lipids and appears to be crucial for both the direct removal of cholesterol-rich LDL from plasma and conversion of IDL particles to LDL particles. Individuals who either lack apolipoprotein E or who are homozygous for particular alleles of apoE may have have a condition known as dysbetalipoproteinemia, which is characterized by elevated plasma cholesterol and triglyceride levels and an increased risk for atherosclerosis.

In a comprehensive review of apoE variants, de Knijff et al., Hum. Mutat. 4:178-194 (1994) found that 30 variants had been characterized, including the most common variant, apoE3. To that time, 14 apoE variants had been found to be associated with familial dysbetalipoproteinemia. The attached table discloses the correcting oligonucleotide base sequences for the APOE oligonucleotides of the invention.

TABLE 23

Apoe Mutations And Genome-Correcting Oligos

| | |
|---|---|
| Clinical Phenotype & | SEQ ID |

| | | |
|---|---|---|
| Mutation | Correcting Oligos | NO: |

| | | |
|---|---|---|
| Apolipoprotein E | TTGTTCCACACAGGATGCCAGGCCAAGGTGGAGCAAGCGGT | 3396 |
| Glu13Lys | GGAGACAGAGCCGGAGCCCGAGCTGCGCCAGCAGACCGAG | |
| cGAG-AAG | TGGCAGAGCGGCCAGCGCTGGGAACTGGCACTGGGTCGCT | |
| | AGCGACCCAGTGCCAGTTCCCAGCGCTGGCCGCTCTGCCAC | 3397 |
| | TCGGTCTGCTGGCGCAGCTCGGGCTCCGGCTCTGTCTCCAC | |
| | CGCTTGCTCCACCTTGGCCTGGCATCCTGTGTGGAACAA | |
| | CGGAGCCCGAGCTGCGC | 3398 |
| | GCGCAGCTCGGGCTCCG | 3399 |
| Apolipoprotein E | CAAGGTGGAGCAAGCGGTGGAGACAGAGCCGGAGCCCGAG | 3400 |
| Trp20Term | CTGCGCCAGCAGACCGAGTGGCAGAGCGGCCAGCGCTGGG | |
| TGGc-TGA | AACTGGCACTGGGTCGCTTTTGGGATTACCTGCGCTGGGTG | |
| | CACCCAGCGCAGGTAATCCCAAAAGCGACCCAGTGCCAGTT | 3401 |
| | CCCAGCGCTGGCCGCTCTGCCACTCGGTCTGCTGGCGCAGC | |
| | TCGGGCTCCGGCTCTGTCTCCACCGCTTGCTCCACCTTG | |
| | ACCGAGTGGCAGAGCGG | 3402 |
| | CCGCTCTGCCACTCGGT | 3403 |
| Apolipoprotein E | CAGAGCCGGAGCCCGAGCTGCGCCAGCAGACCGAGTGGCA | 3404 |
| Leu28Pro | GAGCGGCCAGCGCTGGGAACTGGCACTGGGTGGCTTTTGGG | |
| CTG-CCG | ATTACCTGCGCTGGGTGCAGACACTGTCTGAGCAGGTGCA | |
| | TGCACCTGCTCAGACAGTGTCTGCACCCAGCGCAGGTAATCC | 3405 |
| | CAAAAGCGACCCAGTGCCAGTTCCCAGCGCTGGCCGCTCTG | |
| | CCACTCGGTCTGCTGGCGCAGCTCGGGCTCCGGCTCTG | |
| | CTGGGAACTGGCACTGG | 3406 |
| | CCAGTGCCAGTTCCCAG | 3407 |
| Apolipoprotein E | CGGCTGTCCAAGGAGCTGCAGGCGGCGCAGGCCCGGCTGG | 3408 |
| Cys112Arg | GCGCGGACATGGAGGACGTGTGCGGCCGCCTGGTGCAGTA | |
| gTGC-CGC | CCGCGGCGAGGTGCAGGCCATGCTCGGCCAGAGCACCGAG | |
| | G | |
| | CCTCGGTGCTCTGGCCGAGCATGGCCTGCACCTCGCCGCGG | 3409 |
| | TACTGCACCAGGCGGCCGCACACGTCCTCCATGTCCGCGCC | |
| | CAGCCGGGCCTGCGCCGCCTGCAGCTCCTTGGACAGCCG | |
| | AGGACGTGTGCGGCCGC | 3410 |
| | GCGGCCGCACACGTCCT | 3411 |
| Apolipoprotein E | ACATGGAGGACGTGTGCGGCCGCCTGGTGCAGTACCGCGGC | 3412 |
| Gly127Asp | GAGGTGCAGGCCATGCTCGGCCAGAGCACCGAGGAGCTGC | |
| GGC-GAC | GGGTGCGCCTCGCCTCCCACCTGCGCAAGCTGCGTAAGCG | |
| | CGCTTACGCAGCTTGCGCAGGTGGGAGGCGAGGCGCACCC | 3413 |
| | GCAGCTCCTCGGTGCTCTGGCCGAGCATGGCCTGCACCTCG | |
| | CCGCGGTACTGCACCAGGCGGCCGCACACGTCCTCCATGT | |
| | CATGCTCGGCCAGAGCA | 3414 |
| | TGCTCTGGCCGAGCATG | 3415 |
| Apolipoprotein E | GTGCAGTACCGCGGCGAGGTGCAGGCCATGCTCGGCCAGA | 3416 |
| Arg136Cys | GCACCGAGGAGCTGCGGGTGCGCCTCGCCTCCCACCTGCG | |
| gCGC-TGC | CAAGCTGCGTAAGCGGCTCCTCCGCGATGCCGATGACCTGC | |
| | GCAGGTCATCGGCATCGCGGAGGAGCCGCTTACGCAGCTTG | 3417 |
| | CGCAGGTGGGAGGCGAGGCGCACCCGCAGCTCCTCGGTGC | |
| | TCTGGCCGAGCATGGCCTGCACCTCGCCGCGGTACTGCAC | |
| | TGCGGGTGCGCCTCGCC | 3418 |
| | GGCGAGGCGCACCCGCA | 3419 |
| Apolipoprotein E | TGCAGTACCGCGGCGAGGTGCAGGCCATGCTCGGCCAGAGC | 3420 |
| Arg136His | ACCGAGGAGCTGCGGGTGCGCCTCGCCTCCCACCTGCGCAA | |
| CGC-CAC | GCTGCGTAAGCGGCTCCTCCGCGATGCCGATGACCTGCA | |
| | TGCAGGTCATCGGCATCGCGGAGGAGCCGCTTACGCAGCTT | 3421 |
| | GCGCAGGTGGGAGGCGAGGCGCACCCGCAGCTCCTCGGTG | |
| | CTCTGGCCGAGCATGGCCTGCACCTCGCCGCGGTACTGCA | |
| | GCGGGTGCGCCTCGCCT | 3422 |
| | AGGCGAGGCGCACCCGC | 3423 |
| Apolipoprotein E | GTGCAGTACCGCGGCGAGGTGCAGGCCATGCTCGGCCAGA | 3424 |
| Arg136Ser | GCACCGAGGAGCTGCGGGTGCGCCTCGCCTCCCACCTGCG | |
| gCGC-AGC | CAAGCTGCGTAAGCGGCTCCTCCGCGATGCCGATGACCTGC | |
| | GCAGGTCATCGGCATCGCGGAGGAGCCGCTTACGCAGCTTG | 3425 |
| | CGCAGGTGGGAGGCGAGGCGCACCCGCAGCTCCTCGGTGC | |
| | TCTGGCCGAGCATGGCCTGCACCTCGCCGCGGTACTGCAC | |
| | GCGGGTGCGCCTCGCC | 3426 |
| | GGCGAGGCGCACCCGCA | 3427 |
| Apolipoprotein E | GTGCAGGCCATGCTCGGCCAGAGCACCGAGGAGCTGCGGG | 3428 |
| Arg142Cys | TGCGCCTCGCCTCCCACCTGCGCAAGCTGCGTAAGCGGCTC | |
| gCGC-TGC | CTCCGCGATGCCGATGACCTGCAGAAGCGCCTGGCAGTGT | |
| | ACACTGCCAGGCGCTTCTGCAGGTCATCGGCATCGCGGAGG | 3429 |
| | AGCCGCTTACGCAGCTTGCGCAGGTGGGAGGCGAGGCGCA | |
| | CCCGCAGCTCCTCGGTGCTCTGGCCGAGCATGGCCTGCAC | |
| | CCCACCTGCGCAAGCTG | 3430 |
| | CAGCTTGCGCAGGTGGG | 3431 |
| Apolipoprotein E | TGCAGGCCATGCTCGGCCAGAGCACCGAGGAGCTGCGGGT | 3432 |
| Arg142Leu | GCGCCTCGCCTCCCACCTGCGCAAGCTGCGTAAGCGGCTCC | |
| CGC-CTC | TCCGCGATGCCGATGACCTGCAGAAGCGCCTGGCAGTGTA | |
| | TACACTGCCAGGCGCTTCTGCAGGTCATCGGCATCGCGGAG | 3433 |
| | GAGCCGCTTACGCAGCTTGCGCAGGTGGGAGGCGAGGCGC | |
| | ACCCGCAGCTCCTCGGTGCTCTGGCCGAGCATGGCCTGCA | |
| | CCACCTGCGCAAGCTGC | 3434 |
| | GCAGCTTGCGCAGGTGG | 3435 |
| Apolipoprotein E | ATGCTCGGCCAGAGCACCGAGGAGCTGCGGGTGCGCCTCG | 3436 |
| Arg145Cys | CCTCCCACCTGCGCAAGCTGCGTAAGCGGCTCCTCCGCGAT | |
| gCGT-TGT | GCCGATGACCTGCAGAAGCGCCTGGCAGTGTACCAGGCCG | |
| | CGGCCTGGTACACTGCCAGGCGCTTCTGCAGGTCATCGGCA | 3437 |
| | TCGCGGAGGAGCCGCTTACGCAGCTTGCGCAGGTGGGAGG | |
| | CGAGGCGCACCCGCAGCTCCTCGGTGCTCTGGCCGAGCAT | |
| | GCAAGCTGCGTAAGCGG | 3438 |
| | CCGCTTACGCAGCTTGC | 3439 |
| Apolipoprotein E | TGCTCGGCCAGAGCACCGAGGAGCTGCGGGTGCGCCTCGC | 3440 |
| Arg145Pro | CTCCCACCTGCGCAAGCTGCGTAAGCGGCTCCTCCGCGATG | |
| CGT-CCT | CCGATGACCTGCAGAAGCGCCTGGCAGTGTACCAGGCCGG | |
| | CCGGCCTGGTACACTGCCAGGCGCTTCTGCAGGTCATCGGC | 3441 |
| | ATCGCGGAGGAGCCGCTTACGCAGCTTGCGCAGGTGGGAGG | |
| | CGAGGCGCACCCGCAGCTCCTCGGTGCTCTGGCCGAGCA | |
| | CAAGCTGCGTAAGCGGC | 3442 |
| | GCCGCTTACGCAGCTTG | 3443 |
| Apolipoprotein E | CTCGGCCAGAGCACCGAGGAGCTGCGGGTGCGCCTCGCCT | 3444 |
| Lys146Gln | CCCACCTGCGCAAGCTGCGTAAGCGGCTCCTCCGCGATGCC | |
| tAAG-CAG | GATGACCTGCAGAAGCGCCTGGCAGTGTACCAGGCCGGGG | |
| | CCCCGGCCTGGTACACTGCCAGGCGCTTCTGCAGGTCATCG | 3445 |
| | GCATCGCGGAGGAGCCGCTTACGCAGCTTGCGCAGGTGGGA | |
| | GGCGAGGCGCACCCGCAGCTCCTCGGTGCTCTGGCCGAG | |
| | AGCTGCGTAAGCGGCTC | 3446 |
| | GAGCCGCTTACGCAGCT | 3447 |
| Apolipoprotein E | CTCGGCCAGAGCACCGAGGAGCTGCGGGTGCGCCTCGCCT | 3448 |
| Lys146Glu | CCCACCTGCGCAAGCTGCGTAAGCGGCTCCTCCGCGATGCC | |
| tAAG-GAG | GATGACCTGCAGAAGCGCCTGGCAGTGTACCAGGCCGGGG | |
| | CCCCGGCCTGGTACACTGCCAGGCGCTTCTGCAGGTCATCG | 3449 |
| | GCATCGCGGAGGAGCCGCTTACGCAGCTTGCGCAGGTGGGA | |
| | GGCGAGGCGCACCCGCAGCTCCTCGGTGCTCTGGCCGAG | |
| | AGCTGCGTAAGCGGCTC | 3450 |
| | GAGCCGCTTACGCAGCT | 3451 |
| Apolipoprotein E | GCCTCCCACCTGCGCAAGCTGCGTAAGCGGCTCCTCCGCGA | 3452 |
| Arg158Cys | TGCCGATGACCTGCAGAAGCGCCTGGCAGTGTACCAGGCCG | |
| gCGC-TGC | GGGCCCGCGAGGGCGCCGAGCGCGGCCTCAGCGCCATCC | |
| | GGATGGCGCTGAGGCCGCGCTCGGCGCCCTCGCGGGCCCC | 3453 |
| | GGCCTGGTACACTGCCAGGCGCTTCTGCAGGTCATCGGCAT | |
| | CGCGGAGGAGCCGCTTACGCAGCTTGCGCAGGTGGGAGGC | |
| | TGCAGAAGCGCCTGGCA | 3454 |
| | TGCCAGGCGCTTCTGCA | 3455 |
| Apolipoprotein E | CGCGAGGGCGCCGAGCGCGGCCTCAGCGCCATCCGCGAGC | 3456 |
| Gln187Glu | GCCTGGGGCCCCTGGTGGAACAGGGCCGCGTGCGGGCCGC | |
| aCAG-GAG | CACTGTGGGCTCCCTGGCCGGCCAGCCGCTACAGGAGCGG | |
| | G | |
| | CCCGCTCCTGTAGCGGCTGGCCGGCCAGGGAGCCCACAGT | 3457 |
| | GGCGGCCCGCACGCGGCCCTGTTCCACCAGGGGCCCCAGG | |
| | CGCTCGCGGATGGCGCTGAGGCCGCGCTCGGCGCCCTCGC | |
| | G | |
| | TGGTGGAACAGGGCCGC | 3458 |
| | GCGGCCCTGTTCCACCA | 3459 |
| Apolipoprotein E | TGCGGGCCGCCACTGTGGGCTCCCTGGCCGGCCAGCCGCT | 3460 |
| Trp210Term | ACAGGAGCGGGCCCAGGCCTGGGGCGAGCGGCTGCGCGCG | |
| TGG-TAG | CGGATGGAGGAGATGGGCAGCCGGACCCGCGACCGCCTGG | |
| | A | |
| | TCCAGGCGGTCGCGGGTCCGGCTGCCCATCTCCTCCATCCG | 3461 |
| | CGCGCGCAGCCGCTCGCCCCAGGCCTGGGCCCGCTCCTGT | |
| | AGCGGCTGGCCGGCCAGGGAGCCCACAGTGGCGGCCCGCA | |
| | CCAGGCCTGGGGCGAGC | 3462 |
| | GCTCGCCCCAGGCCTGG | 3463 |
| Apolipoprotein E | CAGGCCTGGGGCGAGCGGCTGCGCGCGCGGATGGAGGAGA | 3464 |
| Arg228Cys | TGGGCAGCCGGACCCGCGACCGCCTGGACGAGGTGAAGGA | |
| cCGC-TGC | GCAGGTGGCGGAGGTGCGCGCCAAGCTGGAGGAGCAGGCC | |
| | C | |
| | GGGCCTGCTCCTCCAGCTTGGCGCGCACCTCCGCCACCTGC | 3465 |
| | TCCTTCACCTCGTCCAGGCGGTCGCGGGTCCGGCTGCCCAT | |
| | CTCCTCCATCCGCGCGCGCAGCCGCTCGCCCCAGGCCTG | |
| | CCCGCGACCGCCTGGAC | 3466 |
| | GTCCAGGCGGTCGCGGG | 3467 |
| Apolipoprotein E | CGGACCCGCGACCGCCTGGACGAGGTGAAGGAGCAGGTGG | 3468 |
| Glu244Lys | CGGAGGTGCGCGCCAAGCTGGAGGAGCAGGCCCAGCAGAT | |
| gGAG-AAG | ACGCCTGCAGGCCGAGGCCTTCCAGGCCCGCCTCAAGAGCT | |
| | AGCTCTTGAGGCGGGCCTGGAAGGCCTCGGCCTGCAGGCGT | 3469 |
| | ATCTGCTGGGCCTGCTCCTCCAGCTTGGCGCGCACCTCCGC | |
| | CACCTGCTCCTTCACCTCGTCCAGGCGGTCGCGGGTCCG | |
| | CCAAGCTGGAGGAGCAG | 3470 |
| | CTGCTCCTCCAGCTTGG | 3471 |

### EXAMPLE 22

### Familial Hypercholesterolemia—LDLR

Familial hypercholesterolemia is characterized by elevation of serum cholesterol bound to low density lipoprotein (LDL) and is, hence, one of the conditions producing a hyperlipoproteinemia phenotype. Familial hypercholesterolemia is an autosomal dominant disorder characterized by elevation of serum cholesterol bound to low density lipoprotein (LDL). Mutations in the LDL receptor (LDLR) gene cause this disorder. The attached table discloses the correcting oligonucleotide base sequences for the LDLR oligonucleotides of the invention.

TABLE 24

LDLR Mutations and Genome-Correcting Oligos

| | |
|---|---|
| Clinical Phenotype & | SEQ ID |

| | | |
|---|---|---|
| Mutation | Correcting Oligos | NO: |

| | | |
|---|---|---|
| Hypercholesterolaemia | GCGTTGAGAGACCCTTTCTCCTTTTCCTCTCTCTCAGTGGGC | 3472 |
| Glu10Term | GACAGATGCGAAAGAAACGAGTTCCAGTGCCAAGACGGGAA | |
| CGAG-TAG | ATGCATCTCCTACAAGTGGGTCTGCGATGGCAGCGCTG | |
| | CAGCGCTGCCATCGCAGACCCACTTGTAGGAGATGCATTTCC | 3473 |
| | CGTCTTGGCACTGGAACTCGTTTCTTTCGCATCTGTCGCCCA | |
| | CTGAGAGAGAGGAAAAGGAGAAAGGGTCTCTCAACGC | |
| | AAAGAAACGAGTTCCAG | 3474 |
| | CTGGAACTCGTTTCTTT | 3475 |
| Hypercholesterolaemia | AGAGACCCTTTCTCCTTTTCCTCTCTCTCAGTGGGCGACAGAT | 3476 |
| Gln12Term | GCGAAAGAAACGAGTTCCAGTGCCAAGACGGGAAATGCATCT | |
| cCAG-TAG | CCTACAAGTGGGTCTGCGATGGCAGCGCTGAGTGCC | |
| | GGCACTCAGCGCTGCCATCGCAGACCCACTTGTAGGAGATG | 3477 |
| | CATTTCCCGTCTTGGCACTGGAACTCGTTTCTTTCGCATCTGT | |
| | CGCCCACTGAGAGAGAGGAAAAGGAGAAAGGGTCTCT | |
| | ACGAGTTCCAGTGCCAA | 3478 |
| | TTGGCACTGGAACTCGT | 3479 |
| Hypercholesterolaemia | CCTTTCTCCTTTTCCTCTCTCTCAGTGGGCGACAGATGCGAAA | 3480 |
| Gln14Term | GAAACGAGTTCCAGTGCCAAGACGGGAAATGCATCTCCTACA | |
| cCAA-TAA | AGTGGGTCTGCGATGGCAGCGCTGAGTGCCAGGATG | |
| | CATCCTGGCACTCAGCGCTGCCATCGCAGACCCACTTGTAGG | 3481 |
| | AGATGCATTTCCCGTCTTGGCACTGGAACTCGTTTCTTTCGCA | |
| | TCTGTCGCCCACTGAGAGAGAGGAAAAGGAGAAAGG | |
| | TCCAGTGCCAAGACGGG | 3482 |
| | CCCGTCTTGGCACTGGA | 3483 |
| Hypercholesterolaemia | GCGACAGATGCGAAAGAAACGAGTTCCAGTGCCAAGACGGG | 3484 |
| Trp23Term | AAATGCATCTCCTACAAGTGGGTCTGCGATGGCAGCGCTGAG | |
| TGG-TAG | TGCCAGGATGGCTCTGATGAGTCCCAGGAGACGTGCTG | |
| | CAGCACGTCTCCTGGGACTCATCAGAGCCATCCTGGCACTCA | 3485 |
| | GCGCTGCCATCGCAGACCCACTTGTAGGAGATGCATTTCCCG | |
| | TCTTGGCACTGGAACTCGTTTCTTTCGCATCTGTCGC | |
| | CTACAAGTGGGTCTGCG | 3486 |
| | CGCAGACCCACTTGTAG | 3487 |
| Hypercholesterolaemia | AACGAGTTCCAGTGCCAAGACGGGAAATGCATCTCCTACAAG | 3488 |
| Ala29Ser | TGGGTCTGCGATGGCAGCGCTGAGTGCCAGGATGGCTCTGA | |
| cGCT-TCT | TGAGTCCCAGGAGACGTGCTGTGAGTCCCCTTTGGGCA | |
| | TGCCCAAAGGGGACTCACAGCACGTCTCCTGGGACTCATCA | 3489 |
| | GAGCCATCCTGGCACTCAGCGCTGCCATCGCAGACCCACTT | |
| | GTAGGAGATGCATTTCCCGTCTTGGCACTGGAACTCGTT | |
| | ATGGCAGCGCTGAGTGC | 3490 |
| | GCACTCAGCGCTGCCAT | 3491 |
| Hypercholesterolaemia | TCCAGTGCCAAGACGGGAAATGCATCTCCTACAAGTGGGTCT | 3492 |
| Cys31Tyr | GCGATGGCAGCGCTGAGTGCCAGGATGGCTCTGATGAGTCC | |
| TGC-TAC | CAGGAGACGTGCTGTGAGTCCCCTTTGGGCATGATATG | |
| | CATATCATGCCCAAAGGGGACTCACAGCACGTCTCCTGGGAC | 3493 |
| | TCATCAGAGCCATCCTGGCACTCAGCGCTGCCATCGCAGACC | |
| | CACTTGTAGGAGATGCATTTCCCGTCTTGGCACTGGA | |
| | CGCTGAGTGCCAGGATG | 3494 |
| | CATCCTGGCACTCAGCG | 3495 |
| Hypercholesterolaemia | AATCCTGTCTCTTCTGTAGTGTCTGTCACCTGCAAATCCGGG | 3496 |
| Arg57Cys | GACTTCAGCTGTGGGGGCCGTGTCAACCGCTGCATTCCTCA | |
| cCGT-TGT | GTTCTGGAGGTGCGATGGCCAAGTGGACTGCGACAACG | |
| | CGTTGTCGCAGTCCACTTGGCCATCGCACCTCCAGAACTGAG | 3497 |
| | GAATGCAGCGGTTGACACGGCCCCCACAGCTGAAGTCCCCG | |
| | GATTTGCAGGTGACAGACACTACAGAAGAGACAGGATT | |
| | GTGGGGGCCGTGTCAAC | 3498 |
| | GTTGACACGGCCCCCAC | 3499 |
| Hypercholesterolaemia | TCTGTCACCTGCAAATCCGGGGACTTCAGCTGTGGGGGCCG | 3500 |
| Gln64Term | TGTCAACCGCTGCATTCCTCAGTTCTGGAGGTGCGATGGCCA | |
| tCAG-TAG | AGTGGACTGCGACAACGGCTCAGACGAGCAAGGCTGTC | |
| | GACAGCCTTGCTCGTCTGAGCCGTTGTCGCAGTCCACTTGGC | 3501 |
| | CATCGCACCTCCAGAACTGAGGAATGCAGCGGTTGACACGG | |
| | CCCCCACAGCTGAAGTCCCCGGATTTGCAGGTGACAGA | |
| | GCATTCCTCAGTTCTGG | 3502 |
| | CCAGAACTGAGGAATGC | 3503 |
| Hypercholesterolaemia | ACCTGCAAATCCGGGGACTTCAGCTGTGGGGGCCGTGTCAA | 3504 |
| Trp66Gly | CCGCTGCATTCCTCAGTTCTGGAGGTGCGATGGCCAAGTGG | |
| cTGG-GGG | ACTGCGACAACGGCTCAGACGAGCAAGGCTGTCGTAAGT | |
| | ACTTACGACAGCCTTGCTCGTCTGAGCCGTTGTCGCAGTCCA | 3505 |
| | CTTGGCCATCGCACCTCCAGAACTGAGGAATGCAGCGGTTG | |
| | ACACGGCCCCCACAGCTGAAGTCCCCGGATTTGCAGGT | |
| | CTCAGTTCTGGAGGTGC | 3506 |
| | GCACCTCCAGAACTGAG | 3507 |
| Hypercholesterolaemia | CCTGCAAATCCGGGGACTTCAGCTGTGGGGGCCGTGTCAAC | 3508 |
| Trp66Term | CGCTGCATTCCTCAGTTCTGGAGGTGCGATGGCCAAGTGGA | |
| TGG-TAG | CTGCGACAACGGCTCAGACGAGCAAGGCTGTCGTAAGTG | |
| | CACTTACGACAGCCTTGCTCGTCTGAGCCGTTGTCGCAGTCC | 3509 |
| | ACTTGGCCATCGCACCTCCAGAACTGAGGAATGCAGCGGTTG | |
| | ACACGGCCCCCACAGCTGAAGTCCCCGGATTTGCAGG | |
| | TCAGTTCTGGAGGTGCG | 3510 |
| | CGCACCTCCAGAACTGA | 3511 |
| Hypercholesterolaemia | AAATCCGGGGACTTCAGCTGTGGGGGCCGTGTCAACCGCTG | 3512 |
| Cys68Arg | CATTCCTCAGTTCTGGAGGTGCGATGGCCAAGTGGACTGCGA | |
| gTGC-CGC | CAACGGCTCAGACGAGCAAGGCTGTCGTAAGTGTGGCC | |
| | GGCCACACTTACGACAGCCTTGCTCGTCTGAGCCGTTGTCGC | 3513 |
| | AGTCCACTTGGCCATCGCACCTCCAGAACTGAGGAATGCAGC | |
| | GGTTGACACGGCCCCCACAGCTGAAGTCCCCGGATTT | |
| | TCTGGAGGTGCGATGGC | 3514 |
| | GCCATCGCACCTCCAGA | 3515 |
| Hypercholesterolaemia | ATCCGGGGACTTCAGCTGTGGGGGCCGTGTCAACCGCTGCA | 3516 |
| Cys68Trp | TTCCTCAGTTCTGGAGGTGCGATGGCCAAGTGGACTGCGACA | |
| TGCg-TGG | ACGGCTCAGACGAGCAAGGCTGTCGTAAGTGTGGCCCT | |
| | AGGGCCACACTTACGACAGCCTTGCTCGTCTGAGCCGTTGTC | 3517 |
| | GCAGTCCACTTGGCCATCGCACCTCCAGAACTGAGGAATGCA | |
| | GCGGTTGACACGGCCCCCACAGCTGAAGTCCCCGGAT | |
| | TGGAGGTGCGATGGCCA | 3518 |
| | TGGCCATCGCACCTCCA | 3519 |
| Hypercholesterolaemia | AATCCGGGGACTTCAGCTGTGGGGGCCGTGTCAACCGCTGC | 3520 |
| Cys68Tyr | ATTCCTCAGTTCTGGAGGTGCGATGGCCAAGTGGACTGCGAC | |
| TGC-TAC | AACGGCTCAGACGAGCAAGGCTGTCGTAAGTGTGGCCC | |
| | GGGCCACACTTACGACAGCCTTGCTCGTCTGAGCCGTTGTCG | 3521 |
| | CAGTCCACTTGGCCATCGCACCTCCAGAACTGAGGAATGCAG | |
| | CGGTTGACACGGCCCCCACAGCTGAAGTCCCCGGATT | |
| | CTGGAGGTGCGATGGCC | 3522 |
| | GGCCATCGCACCTCCAG | 3523 |
| Hypercholesterolaemia | TCCGGGGACTTCAGCTGTGGGGGCCGTGTCAACCGCTGCAT | 3524 |
| Asp69Asn | TCCTCAGTTCTGGAGGTGCGATGGCCAAGTGGACTGCGACA | |
| cGAT-AAT | ACGGCTCAGACGAGCAAGGCTGTCGTAAGTGTGGCCCTG | |
| | CAGGGCCACACTTACGACAGCCTTGCTCGTCTGAGCCGTTGT | 3525 |
| | CGCAGTCCACTTGGCCATCGCACCTCCAGAACTGAGGAATGC | |
| | AGCGGTTGACACGGCCCCCACAGCTGAAGTCCCCGGA | |
| | GGAGGTGCGATGGCCAA | 3526 |
| | TTGGCCATCGCACCTCC | 3527 |
| Hypercholesterolaemia | CCGGGGACTTCAGCTGTGGGGGCCGTGTCAACCGCTGCATT | 3528 |
| Asp69Gly | CCTCAGTTCTGGAGGTGCGATGGCCAAGTGGACTGCGACAA | |
| GAT-GGT | CGGCTCAGACGAGCAAGGCTGTCGTAAGTGTGGCCCTGC | |
| | GCAGGGCCACACTTACGACAGCCTTGCTCGTCTGAGCCGTT | 3529 |
| | GTCGCAGTCCACTTGGCCATCGCACCTCCAGAACTGAGGAAT | |
| | GCAGCGGTTGACACGGCCCCCACAGCTGAAGTCCCCGG | |
| | GAGGTGCGATGGCCAAG | 3530 |
| | CTTGGCCATCGCACCTC | 3531 |
| Hypercholesterolaemia | TCCGGGGACTTCAGCTGTGGGGGCCGTGTCAACCGCTGCAT | 3532 |
| Asp69Tyr | TCCTCAGTTCTGGAGGTGCGATGGCCAAGTGGACTGCGACA | |
| cGAT-TAT | ACGGCTCAGACGAGCAAGGCTGTCGTAAGTGTGGCCCTG | |
| | CAGGGCCACACTTACGACAGCCTTGCTCGTCTGAGCCGTTGT | 3533 |
| | CGCAGTCCACTTGGCCATCGCACCTCCAGAACTGAGGAATGC | |
| | AGCGGTTGACACGGCCCCCACAGCTGAAGTCCCCGGA | |
| | GGAGGTGCGATGGCCAA | 3534 |
| | TTGGCCATCGCACCTCC | 3535 |
| Hypercholesterolaemia | GACTTCAGCTGTGGGGGCCGTGTCAACCGCTGCATTCCTCA | 3536 |
| Gln71Glu | GTTCTGGAGGTGCGATGGCCAAGTGGACTGCGACAACGGCT | |
| cCAA-GAA | CAGACGAGCAAGGCTGTCGTAAGTGTGGCCCTGCCTTTG | |
| | CAAAGGCAGGGCCACACTTACGACAGCCTTGCTCGTCTGAG | 3537 |
| | CCGTTGTCGCAGTCCACTTGGCCATCGCACCTCCAGAACTGA | |
| | GGAATGCAGCGGTTGACACGGCCCCCACAGCTGAAGTC | |
| | GCGATGGCCAAGTGGAC | 3538 |
| | GTCCACTTGGCCATCGC | 3539 |
| Hypercholesterolaemia | TGTGGGGGCCGTGTCAACCGCTGCATTCCTCAGTTCTGGAG | 3540 |
| Cys74Gly | GTGCGATGGCCAAGTGGACTGCGACAACGGCTCAGACGAGC | |
| cTGC-GGC | AAGGCTGTCGTAAGTGTGGCCCTGCCTTTGCTATTGAGC | |
| | GCTCAATAGCAAAGGCAGGGCCACACTTACGACAGCCTTGCT | 3541 |
| | CGTCTGAGCCGTTGTCGCAGTCCACTTGGCCATCGCACCTCC | |
| | AGAACTGAGGAATGCAGCGGTTGACACGGCCCCCACA | |
| | AAGTGGACTGCGACAAC | 3542 |
| | GTTGTCGCAGTCCACTT | 3543 |
| Hypercholesterolaemia | TCAACCGCTGCATTCCTCAGTTCTGGAGGTGCGATGGCCAAG | 3544 |
| Ser78Term | TGGACTGCGACAACGGCTCAGACGAGCAAGGCTGTCGTAAG | |
| TCA-TGA | TGTGGCCCTGCCTTTGCTATTGAGCCTATCTGAGTCCT | |
| | AGGACTCAGATAGGCTCAATAGCAAAGGCAGGGCCACACTTA | 3545 |
| | CGACAGCCTTGCTCGTCTGAGCCGTTGTCGCAGTCCACTTGG | |
| | CCATCGCACCTCCAGAACTGAGGAATGCAGCGGTTGA | |
| | CAACGGCTCAGACGAGC | 3546 |
| | GCTCGTCTGAGCCGTTG | 3547 |
| Hypercholesterolaemia | CGCTGCATTCCTCAGTTCTGGAGGTGCGATGGCCAAGTGGA | 3548 |
| Glu80Lys | CTGCGACAACGGCTCAGACGAGCAAGGCTGTCGTAAGTGTG | |
| cGAG-AAG | GCCCTGCCTTTGCTATTGAGCCTATCTGAGTCCTGGGGA | |
| | TCCCCAGGACTCAGATAGGCTCAATAGCAAAGGCAGGGCCA | 3549 |
| | CACTTACGACAGCCTTGCTCGTCTGAGCCGTTGTCGCAGTCC | |
| | ACTTGGCCATCGCACCTCCAGAACTGAGGAATGCAGCG | |
| | GCTCAGACGAGCAAGGC | 3550 |
| | GCCTTGCTCGTCTGAGC | 3551 |
| Hypercholesterolaemia | CGCTGCATTCCTCAGTTCTGGAGGTGCGATGGCCAAGTGGA | 3552 |
| Glu80Term | CTGCGACAACGGCTCAGACGAGCAAGGCTGTCGTAAGTGTG | |
| cGAG-TAG | GCCCTGCCTTTGCTATTGAGCCTATCTGAGTCCTGGGGA | |
| | TCCCCAGGACTCAGATAGGCTCAATAGCAAAGGCAGGGCCA | 3553 |
| | CACTTACGACAGCCTTGCTCGTCTGAGCCGTTGTCGCAGTCC | |
| | ACTTGGCCATCGCACCTCCAGAACTGAGGAATGCAGCG | |
| | GCTCAGACGAGCAAGGC | 3554 |
| | GCCTTGCTCGTCTGAGC | 3555 |
| Hypercholesterolaemia | TGCATTCCTCAGTTCTGGAGGTGCGATGGCCAAGTGGACTGC | 3556 |
| Gln81Term | GACAACGGCTCAGACGAGCAAGGCTGTCGTAAGTGTGGCCC | |
| gCAA-TAA | TGCCTTTGCTATTGAGCCTATCTGAGTCCTGGGGAGTG | |
| | CACTCCCCAGGACTCAGATAGGCTCAATAGCAAAGGCAGGG | 3557 |
| | CCACACTTACGACAGCCTTGCTCGTCTGAGCCGTTGTCGCAG | |
| | TCCACTTGGCCATCGCACCTCCAGAACTGAGGAATGCA | |
| | CAGACGAGCAAGGCTGT | 3558 |
| | ACAGCCTTGCTCGTCTG | 3559 |
| Hypercholesterolaemia | TGGGAGACTTCACACGGTGATGGTGGTCTCGGCCCATCCATC | 3560 |
| Cys88Arg | CCTGCAGCCCCCAAGACGTGCTCCCAGGACGAGTTTCGCTG | |
| gTGC-CGC | CCACGATGGGAAGTGCATCTCTCGGCAGTTCGTCTGTG | |
| | CACAGACGAACTGCCGAGAGATGCACTTCCCATCGTGGCAG | 3561 |
| | CGAAACTCGTCCTGGGAGCACGTCTTGGGGGCTGCAGGGAT | |
| | GGATGGGCCGAGACCACCATCACCGTGTGAAGTCTCCCA | |
| | CCAAGACGTGCTCCCAG | 3562 |
| | CTGGGAGCACGTCTTGG | 3563 |
| Hypercholesterolaemia | CACGGTGATGGTGGTCTCGGCCCATCCATCCCTGCAGCCCC | 3564 |
| Glu92Term | CAAGACGTGCTCCCAGGACGAGTTTCGCTGCCACGATGGGA | |
| cGAG-TAG | AGTGCATCTCTCGGCAGTTCGTCTGTGACTCAGACCGGG | |
| | CCCGGTCTGAGTCACAGACGAACTGCCGAGAGATGCACTTC | 3565 |
| | CCATCGTGGCAGCGAAACTCGTCCTGGGAGCACGTCTTGGG | |
| | GGCTGCAGGGATGGATGGGCCGAGACCACCATCACCGTG | |
| | CCCAGGACGAGTTTCGC | 3566 |
| | GCGAAACTCGTCCTGGG | 3567 |
| Hypercholesterolaemia | GGTGGTCTCGGCCCATCCATCCCTGCAGCCCCCAAGACGTG | 3568 |
| Cys9SArg | CTCCCAGGACGAGTTTCGCTGCCACGATGGGAAGTGCATCTC | |
| cTGC-CGC | TCGGCAGTTCGTCTGTGACTCAGACCGGGACTGCTTGG | |
| | CCAAGCAGTCCCGGTCTGAGTCACAGACGAACTGCCGAGAG | 3569 |
| | ATGCACTTCCCATCGTGGCAGCGAAACTCGTCCTGGGAGCA | |
| | CGTCTTGGGGGCTGCAGGGATGGATGGGCCGAGACCACC | |
| | AGTTTCGCTGCCACGAT | 3570 |
| | ATCGTGGCAGCGAAACT | 3571 |
| Hypercholesterolaemia | CTCGGCCCATCCATCCCTGCAGCCCCCAAGACGTGCTCCCA | 3572 |
| Asp97Tyr | GGACGAGTTTCGCTGCCACGATGGGAAGTGCATCTCTCGGC | |
| cGAT-TAT | AGTTCGTCTGTGACTCAGACCGGGACTGCTTGGACGGCT | |
| | AGCCGTCCAAGCAGTCCCGGTCTGAGTCACAGACGAACTGC | 3573 |
| | CGAGAGATGCACTTCCCATCGTGGCAGCGAAACTCGTCCTG | |
| | GGAGCACGTCTTGGGGGCTGCAGGGATGGATGGGCCGAG | |
| | GCTGCCACGATGGGAAG | 3574 |
| | CTTCCCATCGTGGCAGC | 3575 |
| Hypercholesterolaemia | GGGTCGGGACACTGCCTGGCAGAGGCTGCGAGCATGGGGC | 3576 |
| Trp(−12)Arg | CCTGGGGCTGGAAATTGCGCTGGACCGTCGCCTTGCTCCTC | |
| cTGG-AGG | GCCGCGGCGGGGACTGCAGGTAAGGCTTGCTCCAGGCGCC | |
| | GGCGCCTGGAGCAAGCCTTACCTGCAGTCCCCGCCGCGGCG | 3577 |
| | AGGAGCAAGGCGACGGTCCAGCGCAATTTCCAGCCCCAGGG | |
| | CCCCATGCTCGCAGCCTCTGCCAGGCAGTGTCCCGACCC | |
| | AATTGCGCTGGACCGTC | 3578 |
| | GACGGTCCAGCGCAATT | 3579 |
| Hypercholesterolaemia | CAGCAGGTCGTGATCCGGGTCGGGACACTGCCTGGCAGAGG | 3580 |
| Trp(−18)Term | CTGCGAGCATGGGGCCCTGGGGCTGGAAATTGCGCTGGACC | |
| TGGg-TGA | GTCGCCTTGCTCCTCGCCGCGGCGGGGACTGCAGGTAAG | |
| | CTTACCTGCAGTCCCCGCCGCGGCGAGGAGCAAGGCGACG | 3581 |
| | GTCCAGCGCAATTTCCAGCCCCAGGGCCCCATGCTCGCAGC | |
| | CTCTGCCAGGCAGTGTCCCGACCCGGATCACGACCTGCTG | |
| | GGGCCCTGGGGCTGGAA | 3582 |
| | TTCCAGCCCCAGGGCCC | 3583 |
| Hypercholesterolaemia | CAGCTAGGACACAGCAGGTCGTGATCCGGGTCGGGACACTG | 3584 |
| Met(−21)Leu | CCTGGCAGAGGCTGCGAGCATGGGGCCCTGGGGCTGGAAA | |
| cATG-TTG | TTGCGCTGGACCGTCGCCTTGCTCCTCGCCGCGGCGGGGA | |
| | TCCCCGCCGCGGCGAGGAGCAAGGCGACGGTCCAGCGCAA | 3585 |
| | TTTCCAGCCCCAGGGCCCCATGCTCGCAGCCTCTGCCAGGC | |
| | AGTGTCCCGACCCGGATCACGACCTGCTGTGTCCTAGCTG | |
| | CTGCGAGCATGGGGCCC | 3586 |
| | GGGCCCCATGCTCGCAG | 3587 |
| Hypercholesterolaemia | CAGCTAGGACACAGCAGGTCGTGATCCGGGTCGGGACACTG | 3588 |
| Met(−21)Val | CCTGGCAGAGGCTGCGAGCATGGGGCCCTGGGGCTGGAAA | |
| cATG-GTG | TTGCGCTGGACCGTCGCCTTGCTCCTCGCCGCGGCGGGGA | |
| | TCCCCGCCGCGGCGAGGAGCAAGGCGACGGTCCAGCGCAA | 3589 |
| | TTTCCAGCCCCAGGGCCCCATGCTCGCAGCCTCTGCCAGGC | |
| | AGTGTCCCGACCCGGATCACGACCTGCTGTGTCCTAGCTG | |
| | CTGCGAGCATGGGGCCC | 3590 |
| | GGGCCCCATGCTCGCAG | 3591 |
| Hypercholesterolaemia | ATCCCTGCAGCCCCCAAGACGTGCTCCCAGGACGAGTTTCG | 3592 |
| Ile101Phe | CTGCCACGATGGGAAGTGCATCTCTCGGCAGTTCGTCTGTGA | |
| cATC-TTC | CTCAGACCGGGACTGCTTGGACGGCTCAGACGAGGCCT | |
| | AGGCCTCGTCTGAGCCGTCCAAGCAGTCCCGGTCTGAGTCA | 3593 |
| | CAGACGAACTGCCGAGAGATGCACTTCCCATCGTGGCAGCG | |
| | AAACTCGTCCTGGGAGCACGTCTTGGGGGCTGCAGGGAT | |
| | GGAAGTGCATCTCTCGG | 3594 |
| | CCGAGAGATGCACTTCC | 3595 |
| Hypercholesterolaemia | GCCCCCAAGACGTGCTCCCAGGACGAGTTTCGCTGCCACGA | 3596 |
| Gln104Term | TGGGAAGTGCATCTCTCGGCAGTTCGTCTGTGACTCAGACCG | |
| gCAG-TAG | GGACTGCTTGGACGGCTCAGACGAGGCCTCCTGCCCGG | |
| | CCGGGCAGGAGGCCTCGTCTGAGCCGTCCAAGCAGTCCCGG | 3597 |
| | TCTGAGTCACAGACGAACTGCCGAGAGATGCACTTCCCATCG | |
| | TGGCAGCGAAACTCGTCCTGGGAGCACGTCTTGGGGGC | |
| | TCTCTCGGCAGTTCGTC | 3598 |
| | GACGAACTGCCGAGAGA | 3599 |
| Hypercholesterolaemia | TTTCGCTGCCACGATGGGAAGTGCATCTCTCGGCAGTTCGTC | 3600 |
| Cys113Arg | TGTGACTCAGACCGGGACTGCTTGGACGGCTCAGACGAGGC | |
| cTGC-CGC | CTCCTGCCCGGTGCTCACCTGTGGTCCCGCCAGCTTCC | |
| | GGAAGCTGGCGGGACCACAGGTGAGCACCGGGCAGGAGGC | 3601 |
| | CTCGTCTGAGCCGTCCAAGCAGTCCCGGTCTGAGTCACAGA | |
| | CGAACTGCCGAGAGATGCACTTCCCATCGTGGCAGCGAAA | |
| | ACCGGGACTGCTTGGAC | 3602 |
| | GTCCAAGCAGTCCCGGT | 3603 |
| Hypercholesterolaemia | AAGTGCATCTCTCGGCAGTTCGTCTGTGACTCAGACCGGGAC | 3604 |
| Glu119Lys | TGCTTGGACGGCTCAGACGAGGCCTCCTGCCCGGTGCTCAC | |
| cGAG-AAG | CTGTGGTCCCGCCAGCTTCCAGTGCAACAGCTCCACCT | |
| | AGGTGGAGCTGTTGCACTGGAAGCTGGCGGGACCACAGGTG | 3605 |
| | AGCACCGGGCAGGAGGCCTCGTCTGAGCCGTCCAAGCAGTC | |
| | CCGGTCTGAGTCACAGACGAACTGCCGAGAGATGCACTT | |
| | GCTCAGACGAGGCCTCC | 3606 |
| | GGAGGCCTCGTCTGAGC | 3607 |
| Hypercholesterolaemia | AAGTGCATCTCTCGGCAGTTCGTCTGTGACTCAGACCGGGAC | 3608 |
| Glu119Term | TGCTTGGACGGCTCAGACGAGGCCTCCTGCCCGGTGCTCAC | |
| cGAG-TAG | CTGTGGTCCCGCCAGCTTCCAGTGCAACAGCTCCACCT | |
| | AGGTGGAGCTGTTGCACTGGAAGCTGGCGGGACCACAGGTG | 3609 |
| | AGCACCGGGCAGGAGGCCTCGTCTGAGCCGTCCAAGCAGTC | |
| | CCGGTCTGAGTCACAGACGAACTGCCGAGAGATGCACTT | |
| | GCTCAGACGAGGCCTCC | 3610 |
| | GGAGGCCTCGTCTGAGC | 3611 |
| Hypercholesterolaemia | TCGGCAGTTCGTCTGTGACTCAGACCGGGACTGCTTGGACG | 3612 |
| Cys122Term | GCTCAGACGAGGCCTCCTGCCCGGTGCTCACCTGTGGTCCC | |
| TGCc-TGA | GCCAGCTTCCAGTGCAACAGCTCCACCTGCATCCCCCAG | |
| | CTGGGGGATGCAGGTGGAGCTGTTGCACTGGAAGCTGGCGG | 3613 |
| | GACCACAGGTGAGCACCGGGCAGGAGGCCTCGTCTGAGCC | |
| | GTCCAAGCAGTCCCGGTCTGAGTCACAGACGAACTGCCGA | |
| | GCCTCCTGCCCGGTGCT | 3614 |
| | AGCACCGGGCAGGAGGC | 3615 |
| Hypercholesterolaemia | TGACTCAGACCGGGACTGCTTGGACGGCTCAGACGAGGCCT | 3616 |
| Cys127Trp | CCTGCCCGGTGCTCACCTGTGGTCCCGCCAGCTTCCAGTGC | |
| TGTg-TGG | AACAGCTCCACCTGCATCCCCCAGCTGTGGGCCTGCGAC | |
| | GTCGCAGGCCCACAGCTGGGGGATGCAGGTGGAGCTGTTGC | 3617 |
| | ACTGGAAGCTGGCGGGACCACAGGTGAGCACCGGGCAGGA | |
| | GGCCTCGTCTGAGCCGTCCAAGCAGTCCCGGTCTGAGTCA | |
| | CTCACCTGTGGTCCCGC | 3618 |
| | GCGGGACCACAGGTGAG | 3619 |
| Hypercholesterolaemia | TGCTTGGACGGCTCAGACGAGGCCTCCTGCCCGGTGCTCAC | 3620 |
| Gln133Term | CTGTGGTCCCGCCAGCTTCCAGTGCAACAGCTCCACCTGCAT | |
| cCAG-TAG | CCCCCAGCTGTGGGCCTGCGACAACGACCCCGACTGCG | |
| | CGCAGTCGGGGTCGTTGTCGCAGGCCCACAGCTGGGGGATG | 3621 |
| | CAGGTGGAGCTGTTGCACTGGAAGCTGGCGGGACCACAGGT | |
| | GAGCACCGGGCAGGAGGCCTCGTCTGAGCCGTCCAAGCA | |
| | CCAGCTTCCAGTGCAAC | 3622 |
| | GTTGCACTGGAAGCTGG | 3623 |
| Hypercholesterolaemia | TTGGACGGCTCAGACGAGGCCTCCTGCCCGGTGCTCACCTG | 3624 |
| Cys134Gly | TGGTCCCGCCAGCTTCCAGTGCAACAGCTCCACCTGCATCCC | |
| gTGC-GGC | CCAGCTGTGGGCCTGCGACAACGACCCCGACTGCGAAG | |
| | CTTCGCAGTCGGGGTCGTTGTCGCAGGCCCACAGCTGGGGG | 3625 |
| | ATGCAGGTGGAGCTGTTGCACTGGAAGCTGGCGGGACCACA | |
| | GGTGAGCACCGGGCAGGAGGCCTCGTCTGAGCCGTCCAA | |
| | GCTTCCAGTGCAACAGC | 3626 |
| | GCTGTTGCACTGGAAGC | 3627 |
| Hypercholesterolaemia | GAGGCCTCCTGCCCGGTGCTCACCTGTGGTCCCGCCAGCTT | 3628 |
| Cys139Gly | CCAGTGCAACAGCTCCACCTGCATCCCCCAGCTGTGGGCCT | |
| cTGC-GGC | GCGACAACGACCCCGACTGCGAAGATGGCTCGGATGAGT | |
| | ACTCATCCGAGCCATCTTCGCAGTCGGGGTCGTTGTCGCAG | 3629 |
| | GCCCACAGCTGGGGGATGCAGGTGGAGCTGTTGCACTGGAA | |
| | GCTGGCGGGACCACAGGTGAGCACCGGGCAGGAGGCCTC | |
| | GCTCCACCTGCATCCCC | 3630 |
| | GGGGATGCAGGTGGAGC | 3631 |
| Hypercholesterolaemia | AGGCCTCCTGCCCGGTGCTCACCTGTGGTCCCGCCAGCTTC | 3632 |
| Cys139Tyr | CAGTGCAACAGCTCCACCTGCATCCCCCAGCTGTGGGCCTG | |
| TGC-TAC | CGACAACGACCCCGACTGCGAAGATGGCTCGGATGAGTG | |
| | CACTCATCCGAGCCATCTTCGCAGTCGGGGTCGTTGTCGCAG | 3633 |
| | GCCCACAGCTGGGGGATGCAGGTGGAGCTGTTGCACTGGAA | |
| | GCTGGCGGGACCACAGGTGAGCACCGGGCAGGAGGCCT | |
| | CTCCACCTGCATCCCCC | 3634 |
| | GGGGGATGCAGGTGGAG | 3635 |
| Hypercholesterolaemia | CTGTGGTCCCGCCAGCTTCCAGTGCAACAGCTCCACCTGCAT | 3636 |
| Cys146Term | CCCCCAGCTGTGGGCCTGCGACAACGACCCCGACTGCGAAG | |
| TGCg-TGA | ATGGCTCGGATGAGTGGCCGCAGCGCTGTAGGGGTCTT | |
| | AAGACCCCTACAGCGCTGCGGCCACTCATCCGAGCCATCTTC | 3637 |
| | GCAGTCGGGGTCGTTGTCGCAGGCCCACAGCTGGGGGATGC | |
| | AGGTGGAGCTGTTGCACTGGAAGCTGGCGGGACCACAG | |
| | TGGGCCTGCGACAACGA | 3638 |
| | TCGTTGTCGCAGGCCCA | 3639 |
| Hypercholesterolaemia | TGTGGTCCCGCCAGCTTCCAGTGCAACAGCTCCACCTGCATC | 3640 |
| Asp147Asn | CCCCAGCTGTGGGCCTGCGACAACGACCCCGACTGCGAAGA | |
| cGAC-AAC | TGGCTCGGATGAGTGGCCGCAGCGCTGTAGGGGTCTTT | |
| | AAAGACCCCTACAGCGCTGCGGCCACTCATCCGAGCCATCTT | 3641 |
| | CGCAGTCGGGGTCGTTGTCGCAGGCCCACAGCTGGGGGATG | |
| | CAGGTGGAGCTGTTGCACTGGAAGCTGGCGGGACCACA | |
| | GGGCCTGCGACAACGAC | 3642 |
| | GTCGTTGTCGCAGGCCC | 3643 |
| Hypercholesterolaemia | TGTGGTCCCGCCAGCTTCCAGTGCAACAGCTCCACCTGCATC | 3644 |
| Asp147His | CCCCAGCTGTGGGCCTGCGACAACGACCCCGACTGCGAAGA | |
| cGAC-CAC | TGGCTCGGATGAGTGGCCGCAGCGCTGTAGGGGTCTTT | |
| | AAAGACCCCTACAGCGCTGCGGCCACTCATCCGAGCCATCTT | 3645 |
| | CGCAGTCGGGGTCGTTGTCGCAGGCCCACAGCTGGGGGATG | |
| | CAGGTGGAGCTGTTGCACTGGAAGCTGGCGGGACCACA | |
| | GGGCCTGCGACAACGAC | 3646 |
| | GTCGTTGTCGCAGGCCC | 3647 |
| Hypercholesterolaemia | TGTGGTCCCGCCAGCTTCCAGTGCAACAGCTCCACCTGCATC | 3648 |
| Asp147Tyr | CCCCAGCTGTGGGCCTGCGACAACGACCCCGACTGCGAAGA | |
| cGAC-TAC | TGGCTCGGATGAGTGGCCGCAGCGCTGTAGGGGTCTTT | |
| | AAAGACCCCTACAGCGCTGCGGCCACTCATCCGAGCCATCTT | 3649 |
| | CGCAGTCGGGGTCGTTGTCGCAGGCCCACAGCTGGGGGATG | |
| | CAGGTGGAGCTGTTGCACTGGAAGCTGGCGGGACCACA | |
| | GGGCCTGCGACAACGAC | 3650 |
| | GTCGTTGTCGCAGGCCC | 3651 |
| Hypercholesterolaemia | TTCCAGTGCAACAGCTCCACCTGCATCCCCCAGCTGTGGGCC | 3652 |
| Cys152Arg | TGCGACAACGACCCCGACTGCGAAGATGGCTCGGATGAGTG | |
| cTGC-CGC | GCCGCAGCGCTGTAGGGGTCTTTACGTGTTCCAAGGGG | |
| | CCCCTTGGAACACGTAAAGACCCCTACAGCGCTGCGGCCAC | 3653 |
| | TCATCCGAGCCATCTTCGCAGTCGGGGTCGTTGTCGCAGGC | |
| | CCACAGCTGGGGGATGCAGGTGGAGCTGTTGCACTGGAA | |
| | ACCCCGACTGCGAAGAT | 3654 |
| | ATCTTCGCAGTCGGGGT | 3655 |
| Hypercholesterolaemia | TTCCAGTGCAACAGCTCCACCTGCATCCCCCAGCTGTGGGCC | 3656 |
| Cys152Gly | TGCGACAACGACCCCGACTGCGAAGATGGCTCGGATGAGTG | |
| cTGC-GGC | GCCGCAGCGCTGTAGGGGTCTTTACGTGTTCCAAGGGG | |
| | CCCCTTGGAACACGTAAAGACCCCTACAGCGCTGCGGCCAC | 3657 |
| | TCATCCGAGCCATCTTCGCAGTCGGGGTCGTTGTCGCAGGC | |
| | CCACAGCTGGGGGATGCAGGTGGAGCTGTTGCACTGGAA | |
| | ACCCCGACTGCGAAGAT | 3658 |
| | ATCTTCGCAGTCGGGGT | 3659 |
| Hypercholesterolaemia | CCAGTGCAACAGCTCCACCTGCATCCCCCAGCTGTGGGCCT | 3660 |
| Cys152Trp | GCGACAACGACCCCGACTGCGAAGATGGCTCGGATGAGTGG | |
| TGCg-TGG | CCGCAGCGCTGTAGGGGTCTTTACGTGTTCCAAGGGGAC | |
| | GTCCCCTTGGAACACGTAAAGACCCCTACAGCGCTGCGGCC | 3661 |
| | ACTCATCCGAGCCATCTTCGCAGTCGGGGTCGTTGTCGCAG | |
| | GCCCACAGCTGGGGGATGCAGGTGGAGCTGTTGCACTGG | |
| | CCCGACTGCGAAGATGG | 3662 |
| | CCATCTTCGCAGTCGGG | 3663 |
| Hypercholesterolaemia | TGCAACAGCTCCACCTGCATCCCCCAGCTGTGGGCCTGCGA | 3664 |
| Asp154Asn | CAACGACCCCGACTGCGAAGATGGCTCGGATGAGTGGCCGC | |
| aGAT-AAT | AGCGCTGTAGGGGTCTTTACGTGTTCCAAGGGGACAGTA | |
| | TACTGTCCCCTTGGAACACGTAAAGACCCCTACAGCGCTGCG | 3665 |
| | GCCACTCATCCGAGCCATCTTCGCAGTCGGGGTCGTTGTCG | |
| | CAGGCCCACAGCTGGGGGATGCAGGTGGAGCTGTTGCA | |
| | ACTGCGAAGATGGCTCG | 3666 |
| | CGAGCCATCTTCGCAGT | 3667 |
| Hypercholesterolaemia | GCTCCACCTGCATCCCCCAGCTGTGGGCCTGCGACAACGAC | 3668 |
| Ser56Leu | CCCGACTGCGAAGATGGCTCGGATGAGTGGCCGCAGCGCTG | |
| TCG-TTG | TAGGGGTCTTTACGTGTTCCAAGGGGACAGTAGCCCCTG | |
| | CAGGGGCTACTGTCCCCTTGGAACACGTAAAGACCCCTACAG | 3669 |
| | CGCTGCGGCCACTCATCCGAGCCATCTTCGCAGTCGGGGTC | |
| | GTTGTCGCAGGCCCACAGCTGGGGGATGCAGGTGGAGC | |
| | AGATGGCTCGGATGAGT | 3670 |
| | ACTCATCCGAGCCATCT | 3671 |
| Hypercholesterolaemia | TGTGGGCCTGCGACAACGACCCCGACTGCGAAGATGGCTCG | 3672 |
| Cys163Tyr | GATGAGTGGCCGCAGCGCTGTAGGGGTCTTTACGTGTTCCAA | |
| TGT-TAT | GGGGACAGTAGCCCCTGCTCGGCCTTCGAGTTCCACTG | |
| | CAGTGGAACTCGAAGGCCGAGCAGGGGCTACTGTCCCCTTG | 3673 |
| | GAACACGTAAAGACCCCTACAGCGCTGCGGCCACTCATCCG | |
| | AGCCATCTTCGCAGTCGGGGTCGTTGTCGCAGGCCCACA | |
| | GCAGCGCTGTAGGGGTC | 3674 |
| | GACCCCTACAGCGCTGC | 3675 |
| Hypercholesterolaemia | CAACGACCCCGACTGCGAAGATGGCTCGGATGAGTGGCCGC | 3676 |
| Tyr167Term | AGCGCTGTAGGGGTCTTTACGTGTTCCAAGGGGACAGTAGC | |
| TACg-TAG | CCCTGCTCGGCCTTCGAGTTCCACTGCCTAAGTGGCGAG | |
| | CTCGCCACTTAGGCAGTGGAACTCGAAGGCCGAGCAGGGGC | 3677 |
| | TACTGTCCCCTTGGAACACGTAAAGACCCCTACAGCGCTGCG | |
| | GCCACTCATCCGAGCCATCTTCGCAGTCGGGGTCGTTG | |
| | GGTCTTTACGTGTTCCA | 3678 |
| | TGGAACACGTAAAGACC | 3679 |
| Hypercholesterolaemia | CCCGACTGCGAAGATGGCTCGGATGAGTGGCCGCAGCGCTG | 3680 |
| Gln170Term | TAGGGGTCTTTACGTGTTCCAAGGGGACAGTAGCCCCTGCTC | |
| cCAA-TAA | GGCCTTCGAGTTCCACTGCCTAAGTGGCGAGTGCATCC | |
| | GGATGCACTCGCCACTTAGGCAGTGGAACTCGAAGGCCGAG | 3681 |
| | CAGGGGCTACTGTCCCCTTGGAACACGTAAAGACCCCTACAG | |
| | CGCTGCGGCCACTCATCCGAGCCATCTTCGCAGTCGGG | |
| | ACGTGTTCCAAGGGGAC | 3682 |
| | GTCCCCTTGGAACACGT | 3683 |
| Hypercholesterolaemia | CGGATGAGTGGCCGCAGCGCTGTAGGGGTCTTTACGTGTTC | 3684 |
| Cys176Phe | CAAGGGGACAGTAGCCCCTGCTCGGCCTTCGAGTTCCACTG | |
| TGC-TTC | CCTAAGTGGCGAGTGCATCCACTCCAGCTGGCGCTGTGA | |
| | TCACAGCGCCAGCTGGAGTGGATGCACTCGCCACTTAGGCA | 3685 |
| | GTGGAACTCGAAGGCCGAGCAGGGGCTACTGTCCCCTTGGA | |
| | ACACGTAAAGACCCCTACAGCGCTGCGGCCACTCATCCG | |
| | TAGCCCCTGCTCGGCCT | 3686 |
| | AGGCCGAGCAGGGGCTA | 3687 |
| Hypercholesterolaemia | CGGATGAGTGGCCGCAGCGCTGTAGGGGTCTTTACGTGTTC | 3688 |
| Cys176Tyr | CAAGGGGACAGTAGCCCCTGCTCGGCCTTCGAGTTCCACTG | |
| TGC-TAC | CCTAAGTGGCGAGTGCATCCACTCCAGCTGGCGCTGTGA | |
| | TCACAGCGCCAGCTGGAGTGGATGCACTCGCCACTTAGGCA | 3689 |
| | GTGGAACTCGAAGGCCGAGCAGGGGCTACTGTCCCCTTGGA | |
| | ACACGTAAAGACCCCTACAGCGCTGCGGCCACTCATCCG | |
| | TAGCCCCTGCTCGGCCT | 3690 |
| | AGGCCGAGCAGGGGCTA | 3691 |
| Hypercholesterolaemia | ATGAGTGGCCGCAGCGCTGTAGGGGTCTTTACGTGTTCCAAG | 3692 |
| Ser177Leu | GGGACAGTAGCCCCTGCTCGGCCTTCGAGTTCCACTGCCTAA | |
| TCG-TTG | GTGGCGAGTGCATCCACTCCAGCTGGCGCTGTGATGG | |
| | CCATCACAGCGCCAGCTGGAGTGGATGCACTCGCCACTTAG | 3693 |
| | GCAGTGGAACTCGAAGGCCGAGCAGGGGCTACTGTCCCCTT | |
| | GGAACACGTAAAGACCCCTACAGCGCTGCGGCCACTCAT | |
| | CCCCTGCTCGGCCTTCG | 3694 |
| | CGAAGGCCGAGCAGGGG | 3695 |
| Hypercholesterolaemia | TACGTGTTCCAAGGGGACAGTAGCCCCTGCTCGGCCTTCGA | 3696 |
| Glu187Lys | GTTCCACTGCCTAAGTGGCGAGTGCATCCACTCCAGCTGGC | |
| cGAG-AAG | GCTGTGATGGTGGCCCCGACTGCAAGGACAAATCTGACG | |
| | CGTCAGATTTGTCCTTGCAGTCGGGGCCACCATCACAGCGCC | 3697 |
| | AGCTGGAGTGGATGCACTCGCCACTTAGGCAGTGGAACTCG | |
| | AAGGCCGAGCAGGGGCTACTGTCCCCTTGGAACACGTA | |
| | TAAGTGGCGAGTGCATC | 3698 |
| | GATGCACTCGCCACTTA | 3699 |
| Hypercholesterolaemia | CAAGGGGACAGTAGCCCCTGCTCGGCCTTCGAGTTCCACTG | 3700 |
| His190Tyr | CCTAAGTGGCGAGTGCATCCACTCCAGCTGGCGCTGTGATG | |
| cCAC-TAC | GTGGCCCCGACTGCAAGGACAAATCTGACGAGGAAAACT | |
| | AGTTTTCCTCGTCAGATTTGTCCTTGCAGTCGGGGCCACCAT | 3701 |
| | CACAGCGCCAGCTGGAGTGGATGCACTCGCCACTTAGGCAG | |
| | TGGAACTCGAAGGCCGAGCAGGGGCTACTGTCCCCTTG | |
| | AGTGCATCCACTCCAGC | 3702 |
| | GCTGGAGTGGATGCACT | 3703 |
| Hypercholesterolaemia | CCTTCGAGTTCCACTGCCTAAGTGGCGAGTGCATCCACTCCA | 3704 |
| Gly198Asp | GCTGGCGCTGTGATGGTGGCCCCGACTGCAAGGACAAATCT | |
| GGC-GAC | GACGAGGAAAACTGCGGTATGGGCGGGGCCAGGGTGGG | |
| | CCCACCCTGGCCCCGCCCATACCGCAGTTTTCCTCGTCAGAT | 3705 |
| | TTGTCCTTGCAGTCGGGGCCACCATCACAGCGCCAGCTGGA | |
| | GTGGATGCACTCGCCACTTAGGCAGTGGAACTCGAAGG | |
| | TGATGGTGGCCCCGACT | 3706 |
| | AGTCGGGGCCACCATCA | 3707 |
| Hypercholesterolaemia | GAGTTCCACTGCCTAAGTGGCGAGTGCATCCACTCCAGCTGG | 3708 |
| Asp200Asn | CGCTGTGATGGTGGCCCCGACTGCAAGGACAAATCTGACGA | |
| cGAC-AAC | GGAAAACTGCGGTATGGGCGGGGCCAGGGTGGGGGCGG | |
| | CCGCCCCCACCCTGGCCCCGCCCATACCGCAGTTTTCCTCG | 3709 |
| | TCAGATTTGTCCTTGCAGTCGGGGCCACCATCACAGCGCCAG | |
| | CTGGAGTGGATGCACTCGCCACTTAGGCAGTGGAACTC | |
| | GTGGCCCCGACTGCAAG | 3710 |
| | CTTGCAGTCGGGGCCAC | 3711 |
| Hypercholesterolaemia | AGTTCCACTGCCTAAGTGGCGAGTGCATCCACTCCAGCTGGC | 3712 |
| Asp200Gly | GCTGTGATGGTGGCCCCGACTGCAAGGACAAATCTGACGAG | |
| GAC-GGC | GAAAACTGCGGTATGGGCGGGGCCAGGGTGGGGGCGGG | |
| | CCCGCCCCCACCCTGGCCCCGCCCATACCGCAGTTTTCCTC | 3713 |
| | GTCAGATTTGTCCTTGCAGTCGGGGCCACCATCACAGCGCCA | |
| | GCTGGAGTGGATGCACTCGCCACTTAGGCAGTGGAACT | |
| | TGGCCCCGACTGCAAGG | 3714 |
| | CCTTGCAGTCGGGGCCA | 3715 |
| Hypercholesterolaemia | GAGTTCCACTGCCTAAGTGGCGAGTGCATCCACTCCAGCTGG | 3716 |
| Asp200Tyr | NCGCTGTGATGGTGGCCCCGACTGCAAGGACAAATCTGACGA | |
| cGAC-TAC | GGAAAACTGCGGTATGGGCGGGGCCAGGGTGGGGGCGG | |
| | CCGCCCCCACCCTGGCCCCGCCCATACCGCAGTTTTCCTCGT | 3717 |
| | CAGATTTGTCCTTGCAGTCGGGGCCACCATCACAGCGCCAG | |
| | CTGGAGTGGATGCACTCGCCACTTAGGCAGTGGAACTC | |
| | GTGGCCCCGACTGCAAG | 3718 |
| | CTTGCAGTCGGGGCCAC | 3719 |
| Hypercholesterolaemia | CCACTGCCTAAGTGGCGAGTGCATCCACTCCAGCTGGCGCT | 3720 |
| Cys201Term | GTGATGGTGGCCCCGACTGCAAGGACAAATCTGACGAGGAA | |
| TGCa-TGA | AACTGCGGTATGGGCGGGGCCAGGGTGGGGGCGGGGCGT | |
| | ACGCCCCGCCCCCACCCTGGCCCCGCCCATACCGCAGTTTT | 3721 |
| | CCTCGTCAGATTTGTCCTTGCAGTCGGGGCCACCATCACAGC | |
| | GCCAGCTGGAGTGGATGCACTCGCCACTTAGGCAGTGG | |
| | CCCGACTGCAAGGACAA | 3722 |
| | TTGTCCTTGCAGTCGGG | 3723 |
| Hypercholesterolaemia | TCCACTGCCTAAGTGGCGAGTGCATCCACTCCAGCTGGCGCT | 3724 |
| Cys201Tyr | GTGATGGTGGCCCCGACTGCAAGGACAAATCTGACGAGGAA | |
| TGC-TAC | AACTGCGGTATGGGCGGGGCCAGGGTGGGGGCGGGGCG | |
| | CGCCCCGCCCCCACCCTGGCCCCGCCCATACCGCAGTTTTC | 3725 |
| | CTCGTCAGATTTGTCCTTGCAGTCGGGGCCACCATCACAGCG | |
| | CCAGCTGGAGTGGATGCACTCGCCACTTAGGCAGTGGA | |
| | CCCCGACTGCAAGGACA | 3726 |
| | TGTCCTTGCAGTCGGGG | 3727 |
| Hypercholesterolaemia | TGCCTAAGTGGCGAGTGCATCCACTCCAGCTGGCGCTGTGAT | 3728 |
| Asp203Asn | GGTGGCCCCGACTGCAAGGACAAATCTGACGAGGAAAACTG | |
| gGAC-AAC | CGGTATGGGCGGGGCCAGGGTGGGGGCGGGGCGTCCTA | |
| | TAGGACGCCCCGCCCCCACCCTGGCCCCGCCCATACCGCAG | 3729 |
| | TTTTCCTCGTCAGATTTGTCCTTGCAGTCGGGGCCACCATCA | |
| | CAGCGCCAGCTGGAGTGGATGCACTCGCCACTTAGGCA | |
| | ACTGCAAGGACAAATCT | 3730 |
| | AGATTTGTCCTTGCAGT | 3731 |
| Hypercholesterolaemia | GCCTAAGTGGCGAGTGCATCCACTCCAGCTGGCGCTGTGAT | 3732 |
| Asp203Gly | GGTGGCCCCGACTGCAAGGACAAATCTGACGAGGAAAACTG | |
| GAC-GGC | CGGTATGGGCGGGGCCAGGGTGGGGGCGGGGCGTCCTAT | |
| | ATAGGACGCCCCGCCCCCACCCTGGCCCCGCCCATACCGCA | 3733 |
| | GTTTTCCTCGTCAGATTTGTCCTTGCAGTCGGGGCCACCATC | |
| | ACAGCGCCAGCTGGAGTGGATGCACTCGCCACTTAGGC | |
| | CTGCAAGGACAAATCTG | 3734 |
| | CAGATTTGTCCTTGCAG | 3735 |
| Hypercholesterolaemia | GCCTAAGTGGCGAGTGCATCCACTCCAGCTGGCGCTGTGAT | 3736 |
| Asp203Val | GGTGGCCCCGACTGCAAGGACAAATCTGACGAGGAAAACTG | |
| GAC-GTC | CGGTATGGGCGGGGCCAGGGTGGGGGCGGGGCGTCCTAT | |
| | ATAGGACGCCCCGCCCCCACCCTGGCCCCGCCCATACCGCA | 3737 |
| | GTTTTCCTCGTCAGATTTGTCCTTGCAGTCGGGGCCACCATC | |
| | ACAGCGCCAGCTGGAGTGGATGCACTCGCCACTTAGGC | |
| | CTGCAAGGACAAATCTG | 3738 |
| | CAGATTTGTCCTTGCAG | 3739 |
| Hypercholesterolaemia | AGTGGCGAGTGCATCCACTCCAGCTGGCGCTGTGATGGTGG | 3740 |
| Ser205Pro | CCCCGACTGCAAGGACAAATCTGACGAGGAAAACTGCGGTAT | |
| aTCT-CCT | GGGCGGGGCCAGGGTGGGGGCGGGGCGTCCTATCACCT | |
| | AGGTGATAGGACGCCCCGCCCCCACCCTGGCCCCGCCCATA | 3741 |
| | CCGCAGTTTTCCTCGTCAGATTTGTCCTTGCAGTCGGGGCCA | |
| | CCATCACAGCGCCAGCTGGAGTGGATGCACTCGCCACT | |
| | AGGACAAATCTGACGAG | 3742 |
| | CTCGTCAGATTTGTCCT | 3743 |
| Hypercholesterolaemia | CGAGTGCATCCACTCCAGCTGGCGCTGTGATGGTGGCCCCG | 3744 |
| Asp206Glu | ACTGCAAGGACAAATCTGACGAGGAAAACTGCGGTATGGGC | |
| GACg-GAG | GGGGCCAGGGTGGGGGCGGGGCGTCCTATCACCTGTCCC | |
| | GGGACAGGTGATAGGACGCCCCGCCCCCACCCTGGCCCCG | 3745 |
| | CCCATACCGCAGTTTTCCTCGTCAGATTTGTCCTTGCAGTCG | |
| | GGGCCACCATCACAGCGCCAGCTGGAGTGGATGCACTCG | |
| | AAATCTGACGAGGAAAA | 3746 |
| | TTTTCCTCGTCAGATTT | 3747 |
| Hypercholesterolaemia | GAGTGCATCCACTCCAGCTGGCGCTGTGATGGTGGCCCCGA | 3748 |
| Glu207Gln | CTGCAAGGACAAATCTGACGAGGAAAACTGCGGTATGGGCG | |
| cGAG-CAG | GGGCCAGGGTGGGGGCGGGGCGTCCTATCACCTGTCCCT | |
| | AGGGACAGGTGATAGGACGCCCCGCCCCCACCCTGGCCCC | 3749 |
| | GCCCATACCGCAGTTTTCCTCGTCAGATTTGTCCTTGCAGTC | |
| | GGGGCCACCATCACAGCGCCAGCTGGAGTGGATGCACTC | |
| | AATCTGACGAGGAAAAC | 3750 |
| | GTTTTCCTCGTCAGATT | 3751 |
| Hypercholesterolaemia | GAGTGCATCCACTCCAGCTGGCGCTGTGATGGTGGCCCCGA | 3752 |
| Glu207Lys | CTGCAAGGACAAATCTGACGAGGAAAACTGCGGTATGGGCG | |
| cGAG-AAG | GGGCCAGGGTGGGGGCGGGGCGTCCTATCACCTGTCCCT | |
| | AGGGACAGGTGATAGGACGCCCCGCCCCCACCCTGGCCCC | 3753 |
| | GCCCATACCGCAGTTTTCCTCGTCAGATTTGTCCTTGCAGTC | |
| | GGGGCCACCATCACAGCGCCAGCTGGAGTGGATGCACTC | |
| | AATCTGACGAGGAAAAC | 3754 |
| | GTTTTCCTCGTCAGATT | 3755 |
| Hypercholesterolaemia | GAGTGCATCCACTCCAGCTGGCGCTGTGATGGTGGCCCCGA | 3756 |
| Glu207Term | CTGCAAGGACAAATCTGACGAGGAAAACTGCGGTATGGGCG | |
| cGAG-TAG | GGGCCAGGGTGGGGGCGGGGCGTCCTATCACCTGTCCCT | |
| | AGGGACAGGTGATAGGACGCCCCGCCCCCACCCTGGCCCC | 3757 |
| | GCCCATACCGCAGTTTTCCTCGTCAGATTTGTCCTTGCAGTC | |
| | GGGGCCACCATCACAGCGCCAGCTGGAGTGGATGCACTC | |
| | AATCTGACGAGGAAAAC | 3758 |
| | GTTTTCCTCGTCAGATT | 3759 |
| Hypercholesterolaemia | TCTTGAGAAAATCAACACACTCTGTCCTGTTTTCCAGCTGTGG | 3760 |
| Glu219Lys | CCACCTGTCGCCCTGACGAATTCCAGTGCTCTGATGGAAACT | |
| cGAA-AAA | GCATCCATGGCAGCCGGCAGTGTGACCGGGAATATG | |
| | CATATTCCCGGTCACACTGCCGGCTGCCATGGATGCAGTTTC | 3761 |
| | CATCAGAGCACTGGAATTCGTCAGGGCGACAGGTGGCCACA | |
| | GCTGGAAAACAGGACAGAGTGTGTTGATTTTCTCAAGA | |
| | GCCCTGACGAATTCCAG | 3762 |
| | CTGGAATTCGTCAGGGC | 3763 |
| Hypercholesterolaemia | GAAAATCAACACACTCTGTCCTGTTTTCCAGCTGTGGCCACCT | 3764 |
| Gln221Term | GTCGCCCTGACGAATTCCAGTGCTCTGATGGAAACTGCATCC | |
| cCAG-TAG | ATGGCAGCCGGCAGTGTGACCGGGAATATGACTGCA | |
| | TGCAGTCATATTCCCGGTCACACTGCCGGCTGCCATGGATGC | 3765 |
| | AGTTTCCATCAGAGCACTGGAATTCGTCAGGGCGACAGGTGG | |
| | CCACAGCTGGAAAACAGGACAGAGTGTGTTGATTTTC | |
| | ACGAATTCCAGTGCTCT | 3766 |
| | AGAGCACTGGAATTCGT | 3767 |
| Hypercholesterolaemia | CCTGTTTTCCAGCTGTGGCCACCTGTCGCCCTGACGAATTCC | 3768 |
| Cys227Phe | AGTGCTCTGATGGAAACTGCATCCATGGCAGCCGGCAGTGT | |
| TGC-TTC | GACCGGGAATATGACTGCAAGGACATGAGCGATGAAGT | |
| | ACTTCATCGCTCATGTCCTTGCAGTCATATTCCCGGTCACACT | 3769 |
| | GCCGGCTGCCATGGATGCAGTTTCCATCAGAGCACTGGAATT | |
| | CGTCAGGGCGACAGGTGGCCACAGCTGGAAAACAGG | |
| | TGGAAACTGCATCCATG | 3770 |
| | CATGGATGCAGTTTCCA | 3771 |
| Hypercholesterolaemia | TCGCCCTGACGAATTCCAGTGCTCTGATGGAAACTGCATCCA | 3772 |
| Asp235Glu | TGGCAGCCGGCAGTGTGACCGGGAATATGACTGCAAGGACA | |
| GACc-GAA | TGAGCGATGAAGTTGGCTGCGTTAATGGTGAGCGCTGG | |
| | CCAGCGCTCACCATTAACGCAGCCAACTTCATCGCTCATGTC | 3773 |
| | CTTGCAGTCATATTCCCGGTCACACTGCCGGCTGCCATGGAT | |
| | GCAGTTTCCATCAGAGCACTGGAATTCGTCAGGGCGA | |
| | CAGTGTGACCGGGAATA | 3774 |
| | TATTCCCGGTCACACTG | 3775 |
| Hypercholesterolaemia | GTCGCCCTGACGAATTCCAGTGCTCTGATGGAAACTGCATCC | 3176 |
| Asp235Gly | ATGGCAGCCGGCAGTGTGACCGGGAATATGACTGCAAGGAC | |
| GAC-GGC | ATGAGCGATGAAGTTGGCTGCGTTAATGGTGAGCGCTG | |
| | CAGCGCTCACCATTAACGCAGCCAACTTCATCGCTCATGTCC | 3777 |
| | TTGCAGTCATATTCCCGGTCACACTGCCGGCTGCCATGGATG | |
| | CAGTTTCCATCAGAGCACTGGAATTCGTCAGGGCGAC | |
| | GCAGTGTGACCGGGAAT | 3778 |
| | ATTCCCGGTCACACTGC | 3779 |
| Hypercholesterolaemia | CCTGACGAATTCCAGTGCTCTGATGGAAACTGCATCCATGGC | 3780 |
| Glu237Lys | AGCCGGCAGTGTGACCGGGAATATGACTGCAAGGACATGAG | |
| gGAA-AAA | CGATGAAGTTGGCTGCGTTAATGGTGAGCGCTGGCCAT | |
| | ATGGCCAGCGCTCACCATTAACGCAGCCAACTTCATCGCTCA | 3781 |
| | TGTCCTTGCAGTCATATTCCCGGTCACACTGCCGGCTGCCAT | |
| | GGATGCAGTTTCCATCAGAGCACTGGAATTCGTCAGG | |
| | GTGACCGGGAATATGAC | 3782 |
| | GTCATATTCCCGGTCAC | 3783 |
| Hypercholesterolaemia | TCCAGTGCTCTGATGGAAACTGCATCCATGGCAGCCGGCAGT | 3784 |
| Cys240Phe | GTGACCGGGAATATGACTGCAAGGACATGAGCGATGAAGTT | |
| TGC-TTC | GGCTGCGTTAATGGTGAGCGCTGGCCATCTGGTTTTCC | |
| | GGAAAACCAGATGGCCAGCGCTCACCATTAACGCAGCCAACT | 3785 |
| | TCATCGCTCATGTCCTTGCAGTCATATTCCCGGTCACACTGCC | |
| | GGCTGCCATGGATGCAGTTTCCATCAGAGCACTGGA | |
| | ATATGACTGCAAGGACA | 3786 |
| | TGTCCTTGCAGTCATAT | 3787 |
| Hypercholesterolaemia | AAACTGCATCCATGGCAGCCGGCAGTGTGACCGGGAATATG | 3788 |
| Asp245Glu | ACTGCAAGGACATGAGCGATGAAGTTGGCTGCGTTAATGGTG | |
| GATg-GAA | AGCGCTGGCCATCTGGTTTTCCATCCCCCATTCTCTGT | |
| | ACAGAGAATGGGGGATGGAAAACCAGATGGCCAGCGCTCAC | 3789 |
| | CATTAACGCAGCCAACTTCATCGCTCATGTCCTTGCAGTCATA | |
| | TTCCCGGTCACACTGCCGGCTGCCATGGATGCAGTTT | |
| | ATGAGCGATGAAGTTGG | 3790 |
| | CCAACTTCATCGCTCAT | 3791 |
| Hypercholesterolaemia | ATGGCAGCCGGCAGTGTGACCGGGAATATGACTGCAAGGAC | 3792 |
| Cys249Tyr | ATGAGCGATGAAGTTGGCTGCGTTAATGGTGAGCGCTGGCC | |
| TGC-TAC | ATCTGGTTTTCCATCCCCCATTCTCTGTGCCTTGCTGCT | |
| | AGCAGCAAGGCACAGAGAATGGGGGATGGAAAACCAGATGG | 3793 |
| | CCAGCGCTCACCATTAACGCAGCCAACTTCATCGCTCATGTC | |
| | CTTGCAGTCATATTCCCGGTCACACTGCCGGCTGCCAT | |
| | AGTTGGCTGCGTTAATG | 3794 |
| | CATTAACGCAGCCAACT | 3795 |
| Hypercholesterolaemia | AGGCTCAGACACACCTGACCTTCCTCCTTCCTCTCTCTGGCT | 3796 |
| Glu256Lys | CTCACAGTGACACTCTGCGAGGGACCCAACAAGTTCAAGTGT | |
| cGAG-AAG | CACAGCGGCGAATGCATCACCCTGGACAAAGTCTGCA | |
| | TGCAGACTTTGTCCAGGGTGATGCATTCGCCGCTGTGACACT | 3797 |
| | TGAACTTGTTGGGTCCCTCGCAGAGTGTCACTGTGAGAGCCA | |
| | GAGAGAGGAAGGAGGAAGGTCAGGTGTGTCTGAGCCT | |
| | CACTCTGCGAGGGACCC | 3798 |
| | GGGTCCCTCGCAGAGTG | 3799 |
| Hypercholesterolaemia | CCTCTCTCTGGCTCTCACAGTGACACTCTGCGAGGGACCCAA | 3800 |
| Ser265Arg | CAAGTTCAAGTGTCACAGCGGCGAATGCATCACCCTGGACAA | |
| AGCg-AGA | AGTCTGCAACATGGCTAGAGACTGCCGGGACTGGTCA | |
| | TGACCAGTCCCGGCAGTCTCTAGCCATGTTGCAGACTTTGTC | 3801 |
| | CAGGGTGATGCATTCGCCGCTGTGACACTTGAACTTGTTGGG | |
| | TCCCTCGCAGAGTGTCACTGTGAGAGCCAGAGAGAGG | |
| | TGTCACAGCGGCGAATG | 3802 |
| | CATTCGCCGCTGTGACA | 3803 |
| Hypercholesterolaemia | TCTCTGGCTCTCACAGTGACACTCTGCGAGGGACCCAACAAG | 3804 |
| Glu267Lys | TTCAAGTGTCACAGCGGCGAATGCATCACCCTGGACAAAGTC | |
| cGAA-AAA | TGCAACATGGCTAGAGACTGCCGGGACTGGTCAGATG | |
| | CATCTGACCAGTCCCGGCAGTCTCTAGCCATGTTGCAGACTT | 3805 |
| | TGTCCAGGGTGATGCATTCGCCGCTGTGACACTTGAACTTGT | |
| | TGGGTCCCTCGCAGAGTGTCACTGTGAGAGCCAGAGA | |
| | ACAGCGGCGAATGCATC | 3806 |
| | GATGCATTCGCCGCTGT | 3807 |
| Hypercholesterolaemia | TCTCTGGCTCTCACAGTGACACTCTGCGAGGGACCCAACAAG | 3808 |
| Glu267Term | TTCAAGTGTCACAGCGGCGAATGCATCACCCTGGACAAAGTC | |
| cGAA-TAA | TGCAACATGGCTAGAGACTGCCGGGACTGGTCAGATG | |
| | CATCTGACCAGTCCCGGCAGTCTCTAGCCATGTTGCAGACTT | 3809 |
| | TGTCCAGGGTGATGCATTCGCCGCTGTGACACTTGAACTTGT | |
| | TGGGTCCCTCGCAGAGTGTCACTGTGAGAGCCAGAGA | |
| | ACAGCGGCGAATGCATC | 3810 |
| | GATGCATTCGCCGCTGT | 3811 |
| Hypercholesterolaemia | ACACTCTGCGAGGGACCCAACAAGTTCAAGTGTCACAGCGG | 3812 |
| Lys273Glu | CGAATGCATCACCCTGGACAAAGTCTGCAACATGGCTAGAGA | |
| cAAA-GAA | CTGCCGGGACTGGTCAGATGAACCCATCAAAGAGTGCG | |
| | CGCACTCTTTGATGGGTTCATCTGACCAGTCCCGGCAGTCTC | 3813 |
| | TAGCCATGTTGCAGACTTTGTCCAGGGTGATGCATTCGCCGC | |
| | TGTGACACTTGAACTTGTTGGGTCCCTCGCAGAGTGT | |
| | CCCTGGACAAAGTCTGC | 3814 |
| | GCAGACTTTGTCCAGGG | 3815 |
| Hypercholesterolaemia | CGAGGGACCCAACAAGTTCAAGTGTCACAGCGGCGAATGCA | 3816 |
| Cys275Term | TCACCCTGGACAAAGTCTGCAACATGGCTAGAGACTGCCGG | |
| TGCa-TGA | GACTGGTCAGATGAACCCATCAAAGAGTGCGGTGAGTCT | |
| | AGACTCACCGCACTCTTTGATGGGTTCATCTGACCAGTCCCG | 3817 |
| | GCAGTCTCTAGCCATGTTGCAGACTTTGTCCAGGGTGATGCA | |
| | TTCGCCGCTGTGACACTTGAACTTGTTGGGTCCCTCG | |
| | AAAGTCTGCAACATGGC | 3818 |
| | GCCATGTTGCAGACTTT | 3819 |
| Hypercholesterolaemia | AGTTCAAGTGTCACAGCGGCGAATGCATCACCCTGGACAAAG | 3820 |
| Asp280Gly | TCTGCAACATGGCTAGAGACTGCCGGGACTGGTCAGATGAA | |
| GAC-GGC | CCCATCAAAGAGTGCGGTGAGTCTCGGTGCAGGCGGCT | |
| | AGCCGCCTGCACCGAGACTCACCGCACTCTTTGATGGGTTCA | 3821 |
| | TCTGACCAGTCCCGGCAGTCTCTAGCCATGTTGCAGACTTTG | |
| | TCCAGGGTGATGCATTCGCCGCTGTGACACTTGAACT | |
| | GGCTAGAGACTGCCGGG | 3822 |
| | CCCGGCAGTCTCTAGCC | 3823 |
| Hypercholesterolaemia | TCAAGTGTCACAGCGGCGAATGCATCACCCTGGACAAAGTCT | 3824 |
| Cys281Tyr | GCAACATGGCTAGAGACTGCCGGGACTGGTCAGATGAACCC | |
| TGC-TAC | ATCAAAGAGTGCGGTGAGTCTCGGTGCAGGCGGCTTGC | |
| | GCAAGCCGCCTGCACCGAGACTCACCGCACTCTTTGATGGG | 3825 |
| | TTCATCTGACCAGTCCCGGCAGTCTCTAGCCATGTTGCAGAC | |
| | TTTGTCCAGGGTGATGCATTCGCCGCTGTGACACTTGA | |
| | TAGAGACTGCCGGGACT | 3826 |
| | AGTCCCGGCAGTCTCTA | 3827 |
| Hypercholesterolaemia | TGTCACAGCGGCGAATGCATCACCCTGGACAAAGTCTGCAAC | 3828 |
| Asp283Asn | ATGGCTAGAGACTGCCGGGACTGGTCAGATGAACCCATCAAA | |
| gGAC-AAC | GAGTGCGGTGAGTCTCGGTGCAGGCGGCTTGCAGAGT | |
| | ACTCTGCAAGCCGCCTGCACCGAGACTCACCGCACTCTTTGA | 3829 |
| | TGGGTTCATCTGACCAGTCCCGGCAGTCTCTAGCCATGTTGC | |
| | AGACTTTGTCCAGGGTGATGCATTCGCCGCTGTGACA | |
| | ACTGCCGGGACTGGTCA | 3830 |
| | TGACCAGTCCCGGCAGT | 3831 |
| Hypercholesterolaemia | TCACAGCGGCGAATGCATCACCCTGGACAAAGTCTGCAACAT | 3832 |
| Asp283Glu | GGCTAGAGACTGCCGGGACTGGTCAGATGAACCCATCAAAG | |
| GACt-GAG | AGTGCGGTGAGTCTCGGTGCAGGCGGCTTGCAGAGTTT | |
| | AAACTCTGCAAGCCGCCTGCACCGAGACTCACCGCACTCTTT | 3833 |
| | GATGGGTTCATCTGACCAGTCCCGGCAGTCTCTAGCCATGTT | |
| | GCAGACTTTGTCCAGGGTGATGCATTCGCCGCTGTGA | |
| | TGCCGGGACTGGTCAGA | 3834 |
| | TCTGACCAGTCCCGGCA | 3835 |
| Hypercholesterolaemia | TGTCACAGCGGCGAATGCATCACCCTGGACAAAGTCTGCAAC | 3836 |
| Asp283Tyr | ATGGCTAGAGACTGCCGGGACTGGTCAGATGAACCCATCAAA | |
| gGAC-TAC | GAGTGCGGTGAGTCTCGGTGCAGGCGGCTTGCAGAGT | |
| | ACTCTGCAAGCCGCCTGCACCGAGACTCACCGCACTCTTTGA | 3837 |
| | TGGGTTCATCTGACCAGTCCCGGCAGTCTCTAGCCATGTTGC | |
| | AGACTTTGTCCAGGGTGATGCATTCGCCGCTGTGACA | |
| | ACTGCCGGGACTGGTCA | 3838 |
| | TGACCAGTCCCGGCAGT | 3839 |
| Hypercholesterolaemia | CAGCGGCGAATGCATCACCCTGGACAAAGTCTGCAACATGG | 3840 |
| Trp284Term | CTAGAGACTGCCGGGACTGGTCAGATGAACCCATCAAAGAGT | |
| TGGt-TGA | GCGGTGAGTCTCGGTGCAGGCGGCTTGCAGAGTTTGTG | |
| | CACAAACTCTGCAAGCCGCCTGCACCGAGACTCACCGCACTC | 3841 |
| | TTTGATGGGTTCATCTGACCAGTCCCGGCAGTCTCTAGCCAT | |
| | GTTGCAGACTTTGTCCAGGGTGATGCATTCGCCGCTG | |
| | CGGGACTGGTCAGATGA | 3842 |
| | TCATCTGACCAGTCCCG | 3843 |
| Hypercholesterolaemia | GCGGCGAATGCATCACCCTGGACAAAGTCTGCAACATGGCTA | 3844 |
| Ser285Leu | GAGACTGCCGGGACTGGTCAGATGAACCCATCAAAGAGTGC | |
| TCA-TTA | GGTGAGTCTCGGTGCAGGCGGCTTGCAGAGTTTGTGGG | |
| | CCCACAAACTCTGCAAGCCGCCTGCACCGAGACTCACCGCA | 3845 |
| | CTCTTTGATGGGTTCATCTGACCAGTCCCGGCAGTCTCTAGC | |
| | CATGTTGCAGACTTTGTCCAGGGTGATGCATTCGCCGC | |
| | GGACTGGTCAGATGAAC | 3846 |
| | GTTCATCTGACCAGTCC | 3847 |
| Hypercholesterolaemia | CCCTGGACAAAGTCTGCAACATGGCTAGAGACTGCCGGGAC | 3848 |
| Lys290Arg | TGGTCAGATGAACCCATCAAAGAGTGCGGTGAGTCTCGGTG | |
| AAA-AGA | CAGGCGGCTTGCAGAGTTTGTGGGGAGCCAGGAAAGGGA | |
| | TCCCTTTCCTGGCTCCCCACAAACTCTGCAAGCCGCCTGCAC | 3849 |
| | CGAGACTCACCGCACTCTTTGATGGGTTCATCTGACCAGTCC | |
| | CGGCAGTCTCTAGCCATGTTGCAGACTTTGTCCAGGG | |
| | ACCCATCAAAGAGTGCG | 3850 |
| | CGCACTCTTTGATGGGT | 3851 |
| Hypercholesterolaemia | GGGTAGGGGCCCGAGAGTGACCAGTCTGCATCCCCTGGCCC | 3852 |
| Cys297Phe | TGCGCAGGGACCAACGAATGCTTGGACAACAACGGCGGCTG | |
| TGC-TTC | TTCCCACGTCTGCAATGACCTTAAGATCGGCTACGAGTG | |
| | CACTCGTAGCCGATCTTAAGGTCATTGCAGACGTGGGAACAG | 3853 |
| | CCGCCGTTGTTGTCCAAGCATTCGTTGGTCCCTGCGCAGGGC | |
| | CAGGGGATGCAGACTGGTCACTCTCGGGCCCCTACCC | |
| | CAACGAATGCTTGGACA | 3854 |
| | TGTCCAAGCATTCGTTG | 3855 |
| Hypercholesterolaemia | GGGTAGGGGCCCGAGAGTGACCAGTCTGCATCCCCTGGCCC | 3856 |
| Cys297Tyr | TGCGCAGGGACCAACGAATGCTTGGACAACAACGGCGGCTG | |
| TGC-TAC | TTCCCACGTCTGCAATGACCTTAAGATCGGCTACGAGTG | |
| | CACTCGTAGCCGATCTTAAGGTCATTGCAGACGTGGGAACAG | 3857 |
| | CCGCCGTTGTTGTCCAAGCATTCGTTGGTCCCTGCGCAGGGC | |
| | CAGGGGATGCAGACTGGTCACTCTCGGGCCCCTACCC | |
| | CAACGAATGCTTGGACA | 3858 |
| | TGTCCAAGCATTCGTTG | 3859 |
| Hypercholesterolaemia | TGCATCCCCTGGCCCTGCGCAGGGACCAACGAATGCTTGGA | 3860 |
| His306Tyr | CAACAACGGCGGCTGTTCCCACGTCTGCAATGACCTTAAGAT | |
| cCAC-TAC | CGGCTACGAGTGCCTGTGCCCCGACGGCTTCCAGCTGG | |
| | CCAGCTGGAAGCCGTCGGGGCACAGGCACTCGTAGCCGATC | 3861 |
| | TTAAGGTCATTGCAGACGTGGGAACAGCCGCCGTTGTTGTCC | |
| | AAGCATTCGTTGGTCCCTGCGCAGGGCCAGGGGATGCA | |
| | GCTGTTCCCACGTCTGC | 3862 |
| | GCAGACGTGGGAACAGC | 3863 |
| Hypercholesterolaemia | CCCTGGCCCTGCGCAGGGACCAACGAATGCTTGGACAACAA | 3864 |
| Cys308Gly | CGGCGGCTGTTCCCACGTCTGCAATGACCTTAAGATCGGCTA | |
| cTGC-GGC | CGAGTGCCTGTGCCCCGACGGCTTCCAGCTGGTGGCCC | |
| | GGGCCACCAGCTGGAAGCCGTCGGGGCACAGGCACTCGTA | 3865 |
| | GCCGATCTTAAGGTCATTGCAGACGTGGGAACAGCCGCCGT | |
| | TGTTGTCCAAGCATTCGTTGGTCCCTGCGCAGGGCCAGGG | |
| | CCCACGTCTGCAATGAC | 3866 |
| | GTCATTGCAGACGTGGG | 3867 |
| Hypercholesterolaemia | CCTGGCCCTGCGCAGGGACCAACGAATGCTTGGACAACAAC | 3868 |
| Cys308Tyr | GGCGGCTGTTCCCACGTCTGCAATGACCTTAAGATCGGCTAC | |
| TGC-TAC | GAGTGCCTGTGCCCCGACGGCTTCCAGCTGGTGGCCCA | |
| | TGGGCCACCAGCTGGAAGCCGTCGGGGCACAGGCACTCGTA | 3869 |
| | GCCGATCTTAAGGTCATTGCAGACGTGGGAACAGCCGCCGTT | |
| | GTTGTCCAAGCATTCGTTGGTCCCTGCGCAGGGCCAGG | |
| | CCACGTCTGCAATGACC | 3870 |
| | GGTCATTGCAGACGTGG | 3871 |
| Hypercholesterolaemia | ACCAACGAATGCTTGGACAACAACGGCGGCTGTTCCCACGTC | 3872 |
| Gly314Ser | TGCAATGACCTTAAGATCGGCTACGAGTGCCTGTGCCCCGAC | |
| cGGC-AGC | GGCTTCCAGCTGGTGGCCCAGCGAAGATGCGAAGGTG | |
| | CACCTTCGCATCTTCGCTGGGCCACCAGCTGGAAGCCGTCG | 3873 |
| | GGGCACAGGCACTCGTAGCCGATCTTAAGGTCATTGCAGAC | |
| | GTGGGAACAGCCGCCGTTGTTGTCCAAGCATTCGTTGGT | |
| | TTAAGATCGGCTACGAG | 3874 |
| | CTCGTAGCCGATCTTAA | 3875 |
| Hypercholesterolaemia | CCAACGAATGCTTGGACAACAACGGCGGCTGTTCCCACGTCT | 3876 |
| Gly314Val | GCAATGACCTTAAGATCGGCTACGAGTGCCTGTGCCCCGAC | |
| GGC-GTC | GGCTTCCAGCTGGTGGCCCAGCGAAGATGCGAAGGTGA | |
| | TCACCTTCGCATCTTCGCTGGGCCACCAGCTGGAAGCCGTCG | 3877 |
| | GGGCACAGGCACTCGTAGCCGATCTTAAGGTCATTGCAGACG | |
| | TGGGAACAGCCGCCGTTGTTGTCCAAGCATTCGTTGG | |
| | TAAGATCGGCTACGAGT | 3878 |
| | ACTCGTAGCCGATCTTA | 3879 |
| Hypercholesterolaemia | CGAATGCTTGGACAACAACGGCGGCTGTTCCCACGTCTGCAA | 3880 |
| Tyr315Term | TGACCTTAAGATCGGCTACGAGTGCCTGTGCCCCGACGGCTT | |
| TACg-TAA | CCAGCTGGTGGCCCAGCGAAGATGCGAAGGTGATTTC | |
| | GAAATCACCTTCGCATCTTCGCTGGGCCACCAGCTGGAAGCC | 3881 |
| | GTCGGGGCACAGGCACTCGTAGCCGATCTTAAGGTCATTGCA | |
| | GACGTGGGAACAGCCGCCGTTGTTGTCCAAGCATTCG | |
| | ATCGGCTACGAGTGCCT | 3882 |
| | AGGCACTCGTAGCCGAT | 3883 |
| Hypercholesterolaemia | TGCTTGGACAACAACGGCGGCTGTTCCCACGTCTGCAATGAC | 3884 |
| Cys317Gly | CTTAAGATCGGCTACGAGTGCCTGTGCCCCGACGGCTTCCA | |
| gTGC-GGC | GCTGGTGGCCCAGCGAAGATGCGAAGGTGATTTCCGGG | |
| | CCCGGAAATCACCTTCGCATCTTCGCTGGGCCACCAGCTGGA | 3885 |
| | AGCCGTCGGGGCACAGGCACTCGTAGCCGATCTTAAGGTCA | |
| | TTGCAGACGTGGGAACAGCCGCCGTTGTTGTCCAAGCA | |
| | GCTACGAGTGCCTGTGC | 3886 |
| | GCACAGGCACTCGTAGC | 3887 |
| Hypercholesterolaemia | TGCTTGGACAACAACGGCGGCTGTTCCCACGTCTGCAATGAC | 3888 |
| Cys317Ser | CTTAAGATCGGCTACGAGTGCCTGTGCCCCGACGGCTTCCA | |
| gTGC-AGC | GCTGGTGGCCCAGCGAAGATGCGAAGGTGATTTCCGGG | |
| | CCCGGAAATCACCTTCGCATCTTCGCTGGGCCACCAGCTGGA | 3889 |
| | AGCCGTCGGGGCACAGGCACTCGTAGCCGATCTTAAGGTCA | |
| | TTGCAGACGTGGGAACAGCCGCCGTTGTTGTCCAAGCA | |
| | GCTACGAGTGCCTGTGC | 3890 |
| | GCACAGGCACTCGTAGC | 3891 |
| Hypercholesterolaemia | ACAACGGCGGCTGTTCCCACGTCTGCAATGACCTTAAGATCG | 3892 |
| Pro320Arg | GCTACGAGTGCCTGTGCCCCGACGGCTTCCAGCTGGTGGCC | |
| CCC-CGC | CAGCGAAGATGCGAAGGTGATTTCCGGGTGGGACTGAG | |
| | CTCAGTCCCACCCGGAAATCACCTTCGCATCTTCG CTGGGCC | 3893 |
| | ACCAGCTGGAAGCCGTCGGGGCACAGGCACTCGTAGCCGAT | |
| | CTTAAGGTCATTGCAGACGTGGGAACAGCCGCCGTTGT | |
| | CCTGTGCCCCGACGGCT | 3894 |
| | AGCCGTCGGGGCACAGG | 3895 |
| Hypercholesterolaemia | AACGGCGGCTGTTCCCACGTCTGCAATGACCTTAAGATCGGC | 3896 |
| Asp321Asn | TACGAGTGCCTGTGCCCCGACGGCTTCCAGCTGGTGGCCCA | |
| cGAC-AAC | GCGAAGATGCGAAGGTGATTTCCGGGTGGGACTGAGCC | |
| | GGCTCAGTCCCACCCGGAAATCACCTTCGCATCTTCGCTGGG | 3897 |
| | CCACCAGCTGGAAGCCGTCGGGGCACAGGCACTCGTAGCCG | |
| | ATCTTAAGGTCATTGCAGACGTGGGAACAGCCGCCGTT | |
| | TGTGCCCCGACGGCTTC | 3898 |
| | GAAGCCGTCGGGGCACA | 3899 |
| Hypercholesterolaemia | CGGCGGCTGTTCCCACGTCTGCAATGACCTTAAGATCGGCTA | 3900 |
| Asp321Glu | CGAGTGCCTGTGCCCCGACGGCTTCCAGCTGGTGGCCCAGC | |
| GACg-GAG | GAAGATGCGAAGGTGATTTCCGGGTGGGACTGAGCCCT | |
| | AGGGCTCAGTCCCACCCGGAAATCACCTTCGCATCTTCGCTG | 3901 |
| | GGCCACCAGCTGGAAGCCGTCGGGGCACAGGCACTCGTAGC | |
| | CGATCTTAAGGTCATTGCAGACGTGGGAACAGCCGCCG | |
| | TGCCCCGACGGCTTCCA | 3902 |
| | TGGAAGCCGTCGGGGCA | 3903 |
| Hypercholesterolaemia | GGCGGCTGTTCCCACGTCTGCAATGACCTTAAGATCGGCTAC | 3904 |
| Gly322Ser | GAGTGCCTGTGCCCCGACGGCTTCCAGCTGGTGGCCCAGCG | |
| cGGC-AGC | AAGATGCGAAGGTGATTTCCGGGTGGGACTGAGCCCTG | |
| | CAGGGCTCAGTCCCACCCGGAAATCACCTTCGCATCTTCGCT | 3905 |
| | GGGCCACCAGCTGGAAGCCGTCGGGGCACAGGCACTCGTA | |
| | GCCGATCTTAAGGTCATTGCAGACGTGGGAACAGCCGCC | |
| | GCCCCGACGGCTTCCAG | 3906 |
| | CTGGAAGCCGTCGGGGC | 3907 |
| Hypercholesterolaemia | TGTTCCCACGTCTGCAATGACCTTAAGATCGGCTACGAGTGC | 3908 |
| Gln324Term | CTGTGCCCCGACGGCTTCCAGCTGGTGGCCCAGCGAAGATG | |
| cCAG-TAG | CGAAGGTGATTTCCGGGTGGGACTGAGCCCTGGGCCCC | |
| | GGGGCCCAGGGCTCAGTCCCACCCGGAAATCACCTTCGCAT | 3909 |
| | CTTCGCTGGGCCACCAGCTGGAAGCCGTCGGGGCACAGGCA | |
| | CTCGTAGCCGATCTTAAGGTCATTGCAGACGTGGGAACA | |
| | ACGGCTTCCAGCTGGTG | 3910 |
| | CACCAGCTGGAAGCCGT | 3911 |
| Hypercholesterolaemia | ATGACCTTAAGATCGGCTACGAGTGCCTGTGCCCCGACGGCT | 3912 |
| Arg329Pro | TCCAGCTGGTGGCCCAGCGAAGATGCGAAGGTGATTTCCGG | |
| CGA-CCA | GTGGGACTGAGCCCTGGGCCCCCTCTGCGCTTCCTGAC | |
| | GTCAGGAAGCGCAGAGGGGGCCCAGGGCTCAGTCCCACCC | 3913 |
| | GGAAATCACCTTCGCATCTTCGCTGGGCCACCAGCTGGAAGC | |
| | CGTCGGGGCACAGGCACTCGTAGCCGATCTTAAGGTCAT | |
| | GGCCCAGCGAAGATGCG | 3914 |
| | CGCATCTTCGCTGGGCC | 3915 |
| Hypercholesterolaemia | AATGACCTTAAGATCGGCTACGAGTGCCTGTGCCCCGACGG | 3916 |
| Arg329Term | CTTCCAGCTGGTGGCCCAGCGAAGATGCGAAGGTGATTTCC | |
| gCGA-TGA | GGGTGGGACTGAGCCCTGGGCCCCCTCTGCGCTTCCTGA | |
| | TCAGGAAGCGCAGAGGGGGCCCAGGGCTCAGTCCCACCCG | 3917 |
| | GAAATCACCTTCGCATCTTCGCTGGGCCACCAGCTGGAAGCC | |
| | GTCGGGGCACAGGCACTCGTAGCCGATCTTAAGGTCATT | |
| | TGGCCCAGCGAAGATGC | 3918 |
| | GCATCTTCGCTGGGCCA | 3919 |
| Hypercholesterolaemia | TCTAGCCATTGGGGAAGAGCCTCCCCACCAAGCCTCTTTCTC | 3920 |
| Glu336Lys | TCTCTTCCAGATATCGATGAGTGTCAGGATCCCGACACCTGC | |
| tGAG-AAG | AGCCAGCTCTGCGTGAACCTGGAGGGTGGCTACAAGT | |
| | ACTTGTAGCCACCCTCCAGGTTCACGCAGAGCTGGCTGCAG | 3921 |
| | GTGTCGGGATCCTGACACTCATCGATATCTGGAAGAGAGAGA | |
| | AAGAGGCTTGGTGGGGAGGCTCTTCCCCAATGGCTAGA | |
| | ATATCGATGAGTGTCAG | 3922 |
| | CTGACACTCATCGATAT | 3923 |
| Hypercholesterolaemia | CATTGGGGAAGAGCCTCCCCACCAAGCCTCTTTCTCTCTCTT | 3924 |
| Gln338Term | CCAGATATCGATGAGTGTCAGGATCCCGACACCTGCAGCCAG | |
| tCAG-TAG | CTCTGCGTGAACCTGGAGGGTGGCTACAAGTGCCAGT | |
| | ACTGGCACTTGTAGCCACCCTCCAGGTTCACGCAGAGCTGG | 3925 |
| | CTGCAGGTGTCGGGATCCTGACACTCATCGATATCTGGAAGA | |
| | GAGAGAAAGAGGCTTGGTGGGGAGGCTCTTCCCCAATG | |
| | ATGAGTGTCAGGATCCC | 3926 |
| | GGGATCCTGACACTCAT | 3927 |
| Hypercholesterolaemia | TCCCCACCAAGCCTCTTTCTCTCTCTTCCAGATATCGATGAGT | 3928 |
| Cys343Arg | GTCAGGATCCCGACACCTGCAGCCAGCTCTGCGTGAACCTG | |
| cTGC-CGC | GAGGGTGGCTACAAGTGCCAGTGTGAGGAAGGCTTCC | |
| | GGAAGCCTTCCTCACACTGGCACTTGTAGCCACCCTCCAGGT | 3929 |
| | TCACGCAGAGCTGGCTGCAGGTGTCGGGATCCTGACACTCA | |
| | TCGATATCTGGAAGAGAGAGAAAGAGGCTTGGTGGGGA | |
| | CCGACACCTGCAGCCAG | 3930 |
| | CTGGCTGCAGGTGTCGG | 3931 |
| Hypercholesterolaemia | CAAGCCTCTTTCTCTCTCTTCCAGATATCGATGAGTGTCAGGA | 3932 |
| Gln345Arg | TCCCGACACCTGCAGCCAGCTCTGCGTGAACCTGGAGGGTG | |
| CAG-CGG | GCTACAAGTGCCAGTGTGAGGAAGGCTTCCAGCTGGA | |
| | TCCAGCTGGAAGCCTTCCTCACACTGGCACTTGTAGCCACCC | 3933 |
| | TCCAGGTTCACGCAGAGCTGGCTGCAGGTGTCGGGATCCTG | |
| | ACACTCATCGATATCTGGAAGAGAGAGAAAGAGGCTTG | |
| | CTGCAGCCAGCTCTGCG | 3934 |
| | CGCAGAGCTGGCTGCAG | 3935 |
| Hypercholesterolaemia | TCTTTCTCTCTCTTCCAGATATCGATGAGTGTCAGGATCCCGA | 3936 |
| Cys347Tyr | CACCTGCAGCCAGCTCTGCGTGAACCTGGAGGGTGGCTACA | |
| TGC-TAC | AGTGCCAGTGTGAGGAAGGCTTCCAGCTGGACCCCCA | |
| | TGGGGGTCCAGCTGGAAGCCTTCCTCACACTGGCACTTGTAG | 3937 |
| | CCACCCTCCAGGTTCACGCAGAGCTGGCTGCAGGTGTCGGG | |
| | ATCCTGACACTCATCGATATCTGGAAGAGAGAGAAAGA | |
| | CCAGCTCTGCGTGAACC | 3938 |
| | GGTTCACGCAGAGCTGG | 3939 |
| Hypercholesterolaemia | CTCTTTCTCTCTCTTCCAGATATCGATGAGTGTCAGGATCCCG | 3940 |
| Cys347Arg | ACACCTGCAGCCAGCTCTGCGTGAACCTGGAGGGTGGCTAC | |
| cTGC-CGC | AAGTGCCAGTGTGAGGAAGGCTTCCAGCTGGACCCCC | |
| | GGGGGTCCAGCTGGAAGCCTTCCTCACACTGGCACTTGTAG | 3941 |
| | CCACCCTCCAGGTTCACGCAGAGCTGGCTGCAGGTGTCGGG | |
| | ATCCTGACACTCATCGATATCTGGAAGAGAGAGAAAGAG | |
| | GCCAGCTCTGCGTGAAC | 3942 |
| | GTTCACGCAGAGCTGGC | 3943 |
| Hypercholesterolaemia | CAGATATCGATGAGTGTCAGGATCCCGACACCTGCAGCCAGC | 3944 |
| Gly352Asp | TCTGCGTGAACCTGGAGGGTGGCTACAAGTGCCAGTGTGAG | |
| GGT-GAT | GAAGGCTTCCAGCTGGACCCCCACACGAAGGCCTGCAA | |
| | TTGCAGGCCTTCGTGTGGGGGTCCAGCTGGAAGCCTTCCTC | 3945 |
| | ACACTGGCACTTGTAGCCACCCTCCAGGTTCACGCAGAGCTG | |
| | GCTGCAGGTGTCGGGATCCTGACACTCATCGATATCTG | |
| | CCTGGAGGGTGGCTACA | 3946 |
| | TGTAGCCACCCTCCAGG | 3947 |
| Hypercholesterolaemia | TCGATGAGTGTCAGGATCCCGACACCTGCAGCCAGCTCTGC | 3948 |
| Tyr354Cys | GTGAACCTGGAGGGTGGCTACAAGTGCCAGTGTGAGGAAGG | |
| TAC-TGC | CTTCCAGCTGGACCCCCACACGAAGGCCTGCAAGGCTGT | |
| | ACAGCCTTGCAGGCCTTCGTGTGGGGGTCCAGCTGGAAGCC | 3949 |
| | TTCCTCACACTGGCACTTGTAGCCACCCTCCAGGTTCACGCA | |
| | GAGCTGGCTGCAGGTGTCGGGATCCTGACACTCATCGA | |
| | GGGTGGCTACAAGTGCC | 3950 |
| | GGCACTTGTAGCCACCC | 3951 |
| Hypercholesterolaemia | CAGGATCCCGACACCTGCAGCCAGCTCTGCGTGAACCTGGA | 3952 |
| Cys358Arg | GGGTGGCTACAAGTGCCAGTGTGAGGAAGGCTTCCAGCTGG | |
| gTGT-CGT | ACCCCCACACGAAGGCCTGCAAGGCTGTGGGTGAGCACG | |
| | CGTGCTCACCCACAGCCTTGCAGGCCTTCGTGTGGGGGTCC | 3953 |
| | AGCTGGAAGCCTTCCTCACACTGGCACTTGTAGCCACCCTCC | |
| | AGGTTCACGCAGAGCTGGCTGCAGGTGTCGGGATCCTG | |
| | AGTGCCAGTGTGAGGAA | 3954 |
| | TTCCTCACACTGGCACT | 3955 |
| Hypercholesterolaemia | TGCAGCCAGCTCTGCGTGAACCTGGAGGGTGGCTACAAGTG | 3956 |
| Gln363Term | CCAGTGTGAGGAAGGCTTCCAGCTGGACCCCCACACGAAGG | |
| cCAG-TAG | CCTGCAAGGCTGTGGGTGAGCACGGGAAGGCGGCGGGTG | |
| | CACCCGCCGCCTTCCCGTGCTCACCCACAGCCTTGCAGGCC | 3957 |
| | TTCGTGTGGGGGTCCAGCTGGAAGCCTTCCTCACACTGGCA | |
| | CTTGTAGCCACCCTCCAGGTTCACGCAGAGCTGGCTGCA | |
| | AAGGCTTCCAGCTGGAC | 3958 |
| | GTCCAGCTGGAAGCCTT | 3959 |

### EXAMPLE 23

### UDP—Glucuronosyltransferase—UGT1

Mutations in the human UGT1 gene result in a range of disease syndromes, ranging from relatively common diseases such as Gilbert's syndrome, which effects up to 7% of the population, to rare disorders such as Crigler-Najjar syndrome. Symptoms of these diseases are the result of diminished bilirubin conjugation and typically present with jaundice or, when mild, as an incidental finding during routing laboratory analysis. Severe cases of Crigler-Najjar syndrome are caused by an absence of UGT1 activity and the majority of these patients die in the neonatal period. The only known treatment is liver transplant. The attached table discloses the correcting oligonucleotide base sequences for the UGT1 oligonucleotides of the invention.

TABLE 25
UGT1 Mutations and Genome-Correcting Oligos

| | |
|---|---|
| Clinical Phenotype & | SEQ ID |

| | | |
|---|---|---|
| Mutation | Correcting Oligos | NO: |

| | | |
|---|---|---|
| Crigler-Najjar syndrome | GCAGGAGCAAAGGCGCCATGGCTGTGGAGTCCCAGGGCGG | 3960 |
| 2 | ACGCCCACTTGTCCTGGGCCTGCTGCTGTGTGTGCTGGGCC | |
| Leu15Arg | CAGTGGTGTCCCATGCTGGGAAGATACTGTTGATCCCAGT | |
| CTG-CGG | ACTGGGATCAACAGTATCTTCCCAGCATGGGACACCACTGGG | 3961 |
| | CCCAGCACACACAGCAGCAGGCCCAGGACAAGTGGGCGTCC | |
| | GCCCTGGGACTCCACAGCCATGGCGCCTTTGCTCCTGC | |
| | CCTGGGCCTGCTGCTGT | 3962 |
| | ACAGCAGCAGGCCCAGG | 3963 |
| Crigler-Najjar syndrome | GGGAAGATACTGTTGATCCCAGTGGATGGCAGCCACTGGCT | 3964 |
| 1 | GAGCATGCTTGGGGCCATCCAGCAGCTGCAGCAGAGGGGAC | |
| Gln49Term | ATGAAATAGTTGTCCTAGCACCTGACGCCTCGTTGTACA | |
| CAG-TAG | TGTACAACGAGGCGTCAGGTGCTAGGACAACTATTTCATGTC | 3965 |
| | CCCTCTGCTGCAGCTGCTGGATGGCCCCAAGCATGCTCAGC | |
| | CAGTGGCTGCCATCCACTGGGATCAACAGTATCTTCCC | |
| | GGGCCATCCAGCAGCTG | 3966 |
| | CAGCTGCTGGATGGCCC | 3967 |
| Crigler-Najjar syndrome | CAGCAGAGGGGACATGAAATAGTTGTCCTAGCACCTGACGCC | 3968 |
| 1 | TCGTTGTACATCAGAGACGGAGCATTTTACACCTTGAAGACG | |
| Gly71Arg | TACCCTGTGCCATTCCAAAGGGAGGATGTGAAAGAGT | |
| GGA-AGA | ACTCTTTCACATCCTCCCTTTGGAATGGCACAGGGTACGTCTT | 3969 |
| | CAAGGTGTAAAATGCTCCGTCTCTGATGTACAACGAGGCGTC | |
| | AGGTGCTAGGACAACTATTTCATGTCCCCTCTGCTG | |
| | TCAGAGACGGAGCATTT | 3970 |
| | AAATGCTCCGTCTCTGA | 3971 |
| Gilbert syndrome | GGGTGAAGAACATGCTCATTGCCTTTTCACAGAACTTTCTGTG | 3972 |
| Pro229Gln | CGACGTGGTTTATTCCCCGTATGCAACCCTTGCCTCAGAATTC | |
| CCG-CAG | CTTCAGAGAGAGGTGACTGTCCAGGACCTATTGAG | |
| | CTCAATAGGTCCTGGACAGTCACCTCTCTCTGAAGGAATTCT | 3973 |
| | GAGGCAAGGGTTGCATACGGGGAATAAACCACGTCGCACAG | |
| | AAAGTTCTGTGAAAAGGCAATGAGCATGTTCTTCACCC | |
| | TTATTCCCCGTATGCAA | 3974 |
| | TTGCATACGGGGAATAA | 3975 |
| Crigler-Najjar syndrome | TGTGAAGGATTACCCTAGGCCCATCATGCCCAATATGGTTTTT | 3976 |
| 1 | GTTGGTGGAATCAACTGCCTTCACCAAAATCCACTATCCCAG | |
| Cys280Term | GTGTGTATTGGAGTGGGACTTTTACATGCGTATATT | |
| TGC-TGA | AATATACGCATGTAAAAGTCCCACTCCAATACACACCTGGGAT | 3977 |
| | AGTGGATTTTGGTGAAGGCAGTTGATTCCACCAACAAAAACC | |
| | ATATTGGGCATGATGGGCCTAGGGTAATCCTTCACA | |
| | ATCAACTGCCTTCACCA | 3978 |
| | TGGTGAAGGCAGTTGAT | 3979 |
| Crigler-Najjar syndrome | ATCAAAGAATATGAGAAAAAATTAACTGAAAATTTTTCTTCTGG | 3980 |
| 1 | CTCTAGGAATTTGAAGCCTACATTAATGCTTCTGGAGAACATG | |
| Ala292Val | GAATTGTGGTTTTCTCTTTGGGATCAATGGTCTC | |
| GCC-GTC | GAGACCATTGATCCCAAAGAGAAAACCACAATTCCATGTTCTC | 3981 |
| | CAGAAGCATTAATGTAGGCTTCAAATTCCTAGAGCCAGAAGA | |
| | AAAATTTTCAGTTAATTTTTTCTCATATTCTTTGAT | |
| | ATTTGAAGCCTACATTA | 3982 |
| | TAATGTAGGCTTCAAAT | 3983 |
| Crigler-Najjar syndrome | AGGAATTTGAAGCCTACATTAATGCTTCTGGAGAACATGGAAT | 3984 |
| 1 | TGTGGTTTTCTCTTTGGGATCAATGGTCTCAGAAATTCCAGAG | |
| Gly308Glu | AAGAAAGCTATGGCAATTGCTGATGCTTTGGGCAA | |
| GGA-GAA | TTGCCCAAAGCATCAGCAATTGCCATAGCTTTCTTCTCTGGAA | 3985 |
| | TTTCTGAGACCATTGATCCCAAAGAGAAAACCACAATTCCATG | |
| | TTCTCCAGAAGCATTAATGTAGGCTTCAAATTCCT | |
| | CTCTTTGGGATCAATGG | 3986 |
| | CCATTGATCCCAAAGAG | 3987 |
| Crigler-Najjar syndrome | GTCTCAGAAATTCCAGAGAAGAAAGCTATGGCAATTGCTGAT | 3988 |
| 1 | GCTTTGGGCAAAATCCCTCAGACAGTAAGAAGATTCTATACCA | |
| Gln331Term | TGGCCTCATATCTATTTTCACAGGAGCGCTAATCCC | |
| CAG-TAG | GGGATTAGCGCTCCTGTGAAAATAGATATGAGGCCATGGTAT | 3989 |
| | AGAATCTTCTTACTGTCTGAGGGATTTTGCCCAAAGCATCAGC | |
| | AATTGCCATAGCTTTCTTCTCTGGAATTTCTGAGAC | |
| | AAATCCCTCAGACAGTA | 3990 |
| | TACTGTCTGAGGGATTT | 3991 |
| Crigler-Najjar syndrome | TCTAATCATATTATGTTCTTTCTTTACGTTCTGCTCTTTTTGCC | 3992 |
| 1 | CCTCCCAGGTCCTGTGGCGGTACACTGGAACCCGACCATCG | |
| Trp335Term | AATCTTGCGAACAACACGATACTTGTTAAGTGGCTA | |
| TGG-TGA | TAGCCACTTAACAAGTATCGTGTTGTTCGCAAGATTCGATGGT | 3993 |
| | CGGGTTCCAGTGTACCGCCACAGGACCTGGGAGGGGCAAAA | |
| | AGAGCAGAACGTAAAGAAAGAACATAATATGATTAGA | |
| | GTCCTGTGGCGGTACAC | 3994 |
| | GTGTACCGCCACAGGAC | 3995 |
| Crigler-Najjar syndrome | ACACTGGAACCCGACCATCGAATCTTGCGAACAACACGATAC | 3996 |
| 1 | TTGTTAAGTGGCTACCCCAAAACGATCTGCTTGGTATGTTGG | |
| Gln357Arg | GCGGATTGGATGTATAGGTCAAACCAGGGTCAAATTA | |
| CAA-CGA | TAATTTGACCCTGGTTTGACCTATACATCCAATCCGCCCAACA | 3997 |
| | TACCAAGCAGATCGTTTTGGGGTAGCCACTTAACAAGTATCG | |
| | TGTTGTTCGCAAGATTCGATGGTCGGGTTCCAGTGT | |
| | GCTACCCCAAAACGATC | 3998 |
| | GATCGTTTTGGGGTAGC | 3999 |
| Crigler-Najjar syndrome | TACACTGGAACCCGACCATCGAATCTTGCGAACAACACGATA | 4000 |
| 1 | CTTGTTAAGTGGCTACCCCAAAACGATCTGCTTGGTATGTTG | |
| Gln357Term | GGCGGATTGGATGTATAGGTCAAACCAGGGTCAAATT | |
| CAA-TAA | AATTTGACCCTGGTTTGACCTATACATCCAATCCGCCCAACAT | 4001 |
| | ACCAAGCAGATCGTTTTGGGGTAGCCACTTAACAAGTATCGT | |
| | GTTGTTCGCAAGATTCGATGGTCGGGTTCCAGTGTA | |
| | GGCTACCCCAAAACGAT | 4002 |
| | ATCGTTTTGGGGTAGCC | 4003 |
| Gilbert syndrome | AACTCAGAGATGTAACTGCTGACATCCTCCCTATTTTGCATCT | 4004 |
| Arg367Gly | CAGGTCACCCGATGACCCGTGCCTTTATCACCCATGCTGGTT | |
| CGT-GGT | CCCATGGTGTTTATGAAAGCATATGCAATGGCGTTC | |
| | GAACGCCATTGCATATGCTTTCATAAACACCATGGGAACCAG | 4005 |
| | CATGGGTGATAAAGGCACGGGTCATCGGGTGACCTGAGATG | |
| | CAAAATAGGGAGGATGTCAGCAGTTACATCTCTGAGTT | |
| | CGATGACCCGTGCCTTT | 4006 |
| | AAAGGCACGGGTCATCG | 4007 |
| Crigler-Najjar syndrome | TCAGAGATGTAACTGCTGACATCCTCCCTATTTTGCATCTCAG | 4008 |
| 1 | GTCACCCGATGACCCGTGCCTTTATCACCCATGCTGGTTCCC | |
| Ala368Thr | ATGGTGTTTATGAAAGCATATGCAATGGCGTTCCCA | |
| GCC-ACC | TGGGAACGCCATTGCATATGCTTTCATAAACACCATGGGAAC | 4009 |
| | CAGCATGGGTGATAAAGGCACGGGTCATCGGGTGACCTGAG | |
| | ATGCAAAATAGGGAGGATGTCAGCAGTTACATCTCTGA | |
| | TGACCCGTGCCTTTATC | 4010 |
| | GATAAAGGCACGGGTCA | 4011 |
| Crigler-Najjar syndrome | CCTCCCTATTTTGCATCTCAGGTCACCCGATGACCCGTGCCT | 4012 |
| 1 | TTATCACCCATGCTGGTTCCCATGGTGTTTATGAAAGCATATG | |
| Ser375Phe | CAATGGCGTTCCCATGGTGATGATGCCCTTGTTTGG | |
| TCC-TTC | CCAAACAAGGGCATCATCACCATGGGAACGCCATTGCATATG | 4013 |
| | CTTTCATAAACACCATGGGAACCAGCATGGGTGATAAAGGCA | |
| | CGGGTCATCGGGTGACCTGAGATGCAAAATAGGGAGG | |
| | TGCTGGTTCCCATGGTG | 4014 |
| | CACCATGGGAACCAGCA | 4015 |
| Crigler-Najjar syndrome | AGGTCACCCGATGACCCGTGCCTTTATCACCCATGCTGGTTC | 4016 |
| 1 | CCATGGTGTTTATGAAAGCATATGCAATGGCGTTCCCATGGT | |
| Ser381Arg | GATGATGCCCTTGTTTGGTGATCAGATGGACAATGCA | |
| AGC-AGG | TGCATTGTCCATCTGATCACCAAACAAGGGCATCATCACCAT | 4017 |
| | GGGAACGCCATTGCATATGCTTTCATAAACACCATGGGAACC | |
| | AGCATGGGTGATAAAGGCACGGGTCATCGGGTGACCT | |
| | TATGAAAGCATATGCAA | 4018 |
| | TTGCATATGCTTTCATA | 4019 |
| Crigler-Najjar syndrome | AGCATATGCAATGGCGTTCCCATGGTGATGATGCCCTTGTTT | 4020 |
| 1 | GGTGATCAGATGGACAATGCAAAGCGCATGGAGACTAAGGG | |
| Ala401Pro | AGCTGGAGTGACCCTGAATGTTCTGGAAATGACTTCTG | |
| GCA-CCA | CAGAAGTCATTTCCAGAACATTCAGGGTCACTCCAGCTCCCT | 4021 |
| | TAGTCTCCATGCGCTTTGCATTGTCCATCTGATCACCAAACAA | |
| | GGGCATCATCACCATGGGAACGCCATTGCATATGCT | |
| | TGGACAATGCAAAGCGC | 4022 |
| | GCGCTTTGCATTGTCCA | 4023 |
| Crigler-Najjar syndrome | GGAGCTGGAGTGACCCTGAATGTTCTGGAAATGACTTCTGAA | 4024 |
| 1 | GATTTAGAAAATGCTCTAAAAGCAGTCATCAATGACAAAAGGT | |
| Lys428Glu | AAGAAAGAAGATACAGAAGAATACTTTGGTCATGGC | |
| AAA-GAA | GCCATGACCAAAGTATTCTTCTGTATCTTCTTTCTTACCTTTTG | 4025 |
| | TCATTGATGACTGCTTTTAGAGCATTTTCTAAATCTTCAGAAGT | |
| | CATTTCCAGAACATTCAGGGTCACTCCAGCTCC | |
| | ATGCTCTAAAAGCAGTC | 4026 |
| | GACTGCTTTTAGAGCAT | 4027 |
| Crigler-Najjar syndrome | ATGAGGCACAAGGGCGCGCCACACCTGCGCCCCGCAGCCCA | 4028 |
| 1 | CGACCTCACCTGGTACCAGTACCATTCCTTGGACGTGATTGG | |
| Tyr486Asp | TTTCCTCTTGGCCGTCGTGCTGACAGTGGCCTTCATCA | |
| TAC-GAC | TGATGAAGGCCACTGTCAGCACGACGGCCAAGAGGAAACCA | 4029 |
| | ATCACGTCCAAGGAATGGTACTGGTACCAGGTGAGGTCGTG | |
| | GGCTGCGGGGCGCAGGTGTGGCGCGCCCTTGTGCCTCAT | |
| | GGTACCAGTACCATTCC | 4030 |
| | GGAATGGTACTGGTACC | 4031 |
| Crigler-Najjar syndrome | ACAAGGGCGCGCCACACCTGCGCCCCGCAGCCCACGACCTC | 4032 |
| 1 | ACCTGGTACCAGTACCATTCCTTGGACGTGATTGGTTTCCTCT | |
| Ser488Phe | TGGCCGTCGTGCTGACAGTGGCCTTCATCACCTTTAA | |
| TCC-TTC | TTAAAGGTGATGAAGGCCACTGTCAGCACGACGGCCAAGAG | 4033 |
| | GAAACCAATCACGTCCAAGGAATGGTACTGGTACCAGGTGAG | |
| | GTCGTGGGCTGCGGGGCGCAGGTGTGGCGCGCCCTTGT | |
| | GTACCATTCCTTGGACG | 4034 |
| | CGTCCAAGGAATGGTAC | 4035 |

### EXAMPLE 24

### Alzheimer's Disease—Amyloid Precursor Protein (APP)

Over the past few decades Alzheimer's disease (AD), once considered a rare disorder, has become recognized as a major public health problem. Although there is no agreement on the exact prevalence of Alzheimer's disease, in part due to difficulties of diagnosis, studies consistently point to an exponential rise in prevalence of this disease with age. After age 65, the percentage of affected people approximately doubles with every decade of life, regardless of definition. Among people age 85 or older, studies suggest that 25 to 35 percent have dementia, including Alzheimer's disease; one study reports that 47.2 percent of people over age 85 have Alzheimer's disease, exclusive of other dementias.

Alzheimer's disease progressively destroys memory, reason, judgment, language, and, eventually, the ability to carry out even the simplest tasks. Anatomic changes associated with Alzheimer's disease begin in the entorhinal cortex, proceed to the hippocampus, and then gradually spread to other regions, particularly the cerebral cortex. Chief among such anatomic changes are the presence of characteristic extracellular plaques and internal neurofibrillary tangles.

At least four genes have been identified to date that contribute to development of Alzheimer's disease: AD1 is caused by mutations in the amyloid precursor gene (APP); AD2 is associated with a particular allele of APOE (see Example 20); AD3 is caused by mutation in a gene encoding a 7-transmembrane domain protein, presenilin-1 (PSEN1), and AD4 is caused by mutation in a gene that encodes a similar 7-transmembrane domain protein, presenilin-2 (PSEN2). The attached table discloses the correcting oligonucleotide base sequences for the APP oligonucleotides of the invention.

TABLE 26
APP Mutations and Genome-Correcting Oligos

| | |
|---|---|
| Clinical Phenotype & | SEQ ID |

| | | |
|---|---|---|
| Mutation | Correcting Oligos | NO: |

| | | |
|---|---|---|
| Alzheimer disease | CTGCATACTTTAATTATGATGTAATACAGGTTCTGGGTTGACA | 4036 |
| Glu665Asp | AATATCAAGACGGAGGAGATCTCTGAAGTGAAGATGGATGCA | |
| GAG-GAC | GAATTCCGACATGACTCAGGATATGAAGTTCATCAT | |
| | ATGATGAACTTCATATCCTGAGTCATGTCGGAATTCTGCATCC | 4037 |
| | ATCTTCACTTCAGAGATCTCCTCCGTCTTGATATTTGTCAACC | |
| | CAGAACCTGTATTACATCATAATTAAAGTATGCAG | |
| | ACGGAGGAGATCTCTGA | 4038 |
| | TCAGAGATCTCCTCCGT | 4039 |
| Alzheimer disease | ATTATATTGCATTTAGAAATTAAAATTCTTTTTCTTAATTTGTTT | 4040 |
| Ala692Gly | TCAAGGTGTTCTTTGCAGAAGATGTGGGTTCAAACAAAGGTG | |
| GCA-GGA | CAATCATTGGACTCATGGTGGGCGGTGTTGTCAT | |
| | ATGACAACACCGCCCACCATGAGTCCAATGATTGCACCTTTG | 4041 |
| | TTTGAACCCACATCTTCTGCAAAGAACACCTTGAAAACAAATT | |
| | AAGAAAAAGAATTTTAATTTCTAAATGCAATATAAT | |
| | GTTCTTTGCAGAAGATG | 4042 |
| | CATCTTCTGCAAAGAAC | 4043 |
| Alzheimer disease | TATATTGCATTTAGAAATTAAAATTCTTTTTCTTAATTTGTTTTC | 4044 |
| Glu693Gln | AAGGTGTTCTTTGCAGAAGATGTGGGTTCAAACAAAGGTGCA | |
| GAA-CAA | ATCATTGGACTCATGGTGGGCGGTGTTGTCATAG | |
| | CTATGACAACACCGCCCACCATGAGTCCAATGATTGCACCTT | 4045 |
| | TGTTTGAACCCACATCTTCTGCAAAGAACACCTTGAAAACAAA | |
| | TTAAGAAAAAGAATTTTAATTTCTAAATGCAATATA | |
| | TCTTTGCAGAAGATGTG | 4046 |
| | CACATCTTCTGCAAAGA | 4047 |
| Alzheimer disease | ATATTGCATTTAGAAATTAAAATTCTTTTTCTTAATTTGTTTTCA | 4048 |
| Glu693Gly | AGGTGTTCTTTGCAGAAGATGTGGGTTCAAACAAAGGTGCAA | |
| GAA-GGA | TCATTGGACTCATGGTGGGCGGTGTTGTCATAGC | |
| | GCTATGACAACACCGCCCACCATGAGTCCAATGATTGCACCT | 4049 |
| | TTGTTTGAACCCACATCTTCTGCAAAGAACACCTTGAAAACAA | |
| | ATTAAGAAAAAGAATTTTAATTTCTAAATGCAATAT | |
| | CTTTGCAGAAGATGTGG | 4050 |
| | CCACATCTTCTGCAAAG | 4051 |
| Alzheimer disease | GAAGATGTGGGTTCAAACAAAGGTGCAATCATTGGACTCATG | 4052 |
| Ala713Thr | GTGGGCGGTGTTGTCATAGCGACAGTGATCGTCATCACCTTG | |
| GCG-ACG | GTGATGCTGAAGAAGAAACAGTACACATCCATTCATC | |
| | GATGAATGGATGTGTACTGTTTCTTCTTCAGCATCACCAAGGT | 4053 |
| | GATGACGATCACTGTCGCTATGACAACACCGCCCACCATGAG | |
| | TCCAATGATTGCACCTTTGTTTGAACCCACATCTTC | |
| | TTGTCATAGCGACAGTG | 4054 |
| | CACTGTCGCTATGACAA | 4055 |
| Schizophrenia | AAGATGTGGGTTCAAACAAAGGTGCAATCATTGGACTCATGG | 4056 |
| Ala713Val | TGGGCGGTGTTGTCATAGCGACAGTGATCGTCATCACCTTGG | |
| GCG-GTG | TGATGCTGAAGAAGAAACAGTACACATCCATTCATCA | |
| | TGATGAATGGATGTGTACTGTTTCTTCTTCAGCATCACCAAGG | 4057 |
| | TGATGACGATCACTGTCGCTATGACAACACCGCCCACCATGA | |
| | GTCCAATGATTGCACCTTTGTTTGAACCCACATCTT | |
| | TGTCATAGCGACAGTGA | 4058 |
| | TCACTGTCGCTATGACA | 4059 |
| Alzheimer disease | GTGGGTTCAAACAAAGGTGCAATCATTGGACTCATGGTGGGC | 4060 |
| Val715Met | GGTGTTGTCATAGCGACAGTGATCGTCATCACCTTGGTGATG | |
| GTG-ATG | CTGAAGAAGAAACAGTACACATCCATTCATCATGGTG | |
| | CACCATGATGAATGGATGTGTACTGTTTCTTCTTCAGCATCAC | 4061 |
| | CAAGGTGATGACGATCACTGTCGCTATGACAACACCGCCCAC | |
| | CATGAGTCCAATGATTGCACCTTTGTTTGAACCCAC | |
| | TAGCGACAGTGATCGTC | 4062 |
| | GACGATCACTGTCGCTA | 4063 |
| Alzheimer disease | GGTTCAAACAAAGGTGCAATCATTGGACTCATGGTGGGCGGT | 4064 |
| Ile716Val | GTTGTCATAGCGACAGTGATCGTCATCACCTTGGTGATGCTG | |
| ATC-GTC | AAGAAGAAACAGTACACATCCATTCATCATGGTGTGG | |
| | CCACACCATGATGAATGGATGTGTACTGTTTCTTCTTCAGCAT | 4065 |
| | CACCAAGGTGATGACGATCACTGTCGCTATGACAACACCGCC | |
| | CACCATGAGTCCAATGATTGCACCTTTGTTTGAACC | |
| | CGACAGTGATCGTCATC | 4066 |
| | GATGACGATCACTGTCG | 4067 |
| Alzheimer disease | CAAACAAAGGTGCAATCATTGGACTCATGGTGGGCGGTGTTG | 4068 |
| Val717Gly | TCATAGCGACAGTGATCGTCATCACCTTGGTGATGCTGAAGA | |
| GTC-GGC | AGAAACAGTACACATCCATTCATCATGGTGTGGTGGA | |
| | TCCACCACACCATGATGAATGGATGTGTACTGTTTCTTCTTCA | 4069 |
| | GCATCACCAAGGTGATGACGATCACTGTCGCTATGACAACAC | |
| | CGCCCACCATGAGTCCAATGATTGCACCTTTGTTTG | |
| | AGTGATCGTCATCACCT | 4070 |
| | AGGTGATGACGATCACT | 4071 |
| Alzheimer disease | TCAAACAAAGGTGCAATCATTGGACTCATGGTGGGCGGTGTT | 4072 |
| Val717Ile | GTCATAGCGACAGTGATCGTCATCACCTTGGTGATGCTGAAG | |
| GTC-ATC | AAGAAACAGTACACATCCATTCATCATGGTGTGGTGG | |
| | CCACCACACCATGATGAATGGATGTGTACTGTTTCTTCTTCAG | 4073 |
| | CATCACCAAGGTGATGACGATCACTGTCGCTATGACAACACC | |
| | GCCCACCATGAGTCCAATGATTGCACCTTTGTTTGA | |
| | CAGTGATCGTCATCACC | 4074 |
| | GGTGATGACGATCACTG | 4075 |
| Alzheimer disease | TCAAACAAAGGTGCAATCATTGGACTCATGGTGGGCGGTGTT | 4076 |
| Val717Phe | GTCATAGCGACAGTGATCGTCATCACCTTGGTGATGCTGAAG | |
| GTC-TTC | AAGAAACAGTACACATCCATTCATCATGGTGTGGTGG | |
| | CCACCACACCATGATGAATGGATGTGTACTGTTTCTTCTTCAG | 4077 |
| | CATCACCAAGGTGATGACGATCACTGTCGCTATGACAACACC | |
| | GCCCACCATGAGTCCAATGATTGCACCTTTGTTTGA | |
| | CAGTGATCGTCATCACC | 4078 |
| | GGTGATGACGATCACTG | 4079 |
| Alzheimer disease | TTGGACTCATGGTGGGCGGTGTTGTCATAGCGACAGTGATCG | 4080 |
| Leu723Pro | TCATCACCTTGGTGATGCTGAAGAAGAAACAGTACACATCCAT | |
| CTG-CCG | TCATCATGGTGTGGTGGAGGTAGGTAAACTTGACTG | |
| | CAGTCAAGTTTACCTACCTCCACCACACCATGATGAATGGAT | 4081 |
| | GTGTACTGTTTCTTCTTCAGCATCACCAAGGTGATGACGATCA | |
| | CTGTCGCTATGACAACACCGCCCACCATGAGTCCAA | |
| | GGTGATGCTGAAGAAGA | 4082 |
| | TCTTCTTCAGCATCACC | 4083 |

### EXAMPLE 25

### Alzheimer's Disease—Presenilin—1 (PSEN1)

The attached table discloses the correcting oligonucleotide base sequences for the PSEN1 oligonucleotides of the invention.

TABLE 27
PSEN1 Mutations and Genome-Correcting Oligos

| | |
|---|---|
| Clinical Phenotype & | SEQ ID |

| | | |
|---|---|---|
| Mutation | Correcting Oligos | NO: |

| | | |
|---|---|---|
| Alzheimer disease | CCCGGCAGGTGGTGGAGCAAGATGAGGAAGAAGATGAGGAG | 4084 |
| Ala79Val | CTGACATTGAAATATGGCGCCAAGCATGTGATCATGCTCTTTG | |
| GCC-GTC | TCCCTGTGACTCTCTGCATGGTGGTGGTCGTGGCTAC | |
| | GTAGCCACGACCACCACCATGCAGAGAGTCACAGGGACAAA | 4085 |
| | GAGCATGATCACATGCTTGGCGCCATATTTCAATGTCAGCTC | |
| | CTCATCTTCTTCCTCATCTTGCTCCACCACCTGCCGGG | |
| | ATATGGCGCCAAGCATG | 4086 |
| | CATGCTTGGCGCCATAT | 4087 |
| Alzheimer disease | GTGGTGGAGCAAGATGAGGAAGAAGATGAGGAGCTGACATT | 4088 |
| Val82Leu | GAAATATGGCGCCAAGCATGTGATCATGCTCTTTGTCCCTGT | |
| tGTG-CTG | GACTCTCTGCATGGTGGTGGTCGTGGCTACCATTAAGT | |
| | ACTTAATGGTAGCCACGACCACCACCATGCAGAGAGTCACAG | 4089 |
| | GGACAAAGAGCATGATCACATGCTTGGCGCCATATTTCAATG | |
| | TCAGCTCCTCATCTTCTTCCTCATCTTGCTCCACCAC | |
| | CCAAGCATGTGATCATG | 4090 |
| | CATGATCACATGCTTGG | 4091 |
| Alzheimer disease | AAATATGGCGCCAAGCATGTGATCATGCTCTTTGTCCCTGTG | 4092 |
| Val96Phe | ACTCTCTGCATGGTGGTGGTCGTGGCTACCATTAAGTCAGTC | |
| gGTC-TTC | AGCTTTTATACCCGGAAGGATGGGCAGCTGTACGTAT | |
| | ATACGTACAGCTGCCCATCCTTCCGGGTATAAAAGCTGACTG | 4093 |
| | ACTTAATGGTAGCCACGACCACCACCATGCAGAGAGTCACAG | |
| | GGACAAAGAGCATGATCACATGCTTGGCGCCATATTT | |
| | TGGTGGTGGTCGTGGCT | 4094 |
| | AGCCACGACCACCACCA | 4095 |
| Alzheimer disease | CTTTGTCCCTGTGACTCTCTGCATGGTGGTGGTCGTGGCTAC | 4096 |
| Phe105Leu | CATTAAGTCAGTCAGCTTTTATACCCGGAAGGATGGGCAGCT | |
| TTTt-TTG | GTACGTATGAGTTTTGTTTTATTATTCTCAAAGCCAG | |
| | CTGGCTTTGAGAATAATAAAACAAAACTCATACGTACAGCTGC | 4097 |
| | CCATCCTTCCGGGTATAAAAGCTGACTGACTTAATGGTAGCC | |
| | ACGACCACCACCATGCAGAGAGTCACAGGGACAAAG | |
| | GTCAGCTTTTATACCCG | 4098 |
| | CGGGTATAAAAGCTGAC | 4099 |
| Alzheimer disease | TGGTGATCTCCATTAACACTGACCTAGGGCTTTTGTGTTTGTT | 4100 |
| Thr116Asn | TTATTGTAGAATCTATACCCCATTCACAGAAGATACCGAGACT | |
| ACC-AAC | GTGGGCCAGAGAGCCCTGCACTCAATTCTGAATGC | |
| | GCATTCAGAATTGAGTGCAGGGCTCTCTGGCCCACAGTCTCG | 4101 |
| | GTATCTTCTGTGAATGGGGTATAGATTCTACAATAAAACAAAC | |
| | ACAAAAGCCCTAGGTCAGTGTTAATGGAGATCACCA | |
| | AATCTATACCCCATTCA | 4102 |
| | TGAATGGGGTATAGATT | 4103 |
| Alzheimer disease | TGATCTCCATTAACACTGACCTAGGGCTTTTGTGTTTGTTTTAT | 4104 |
| Pro117Leu | TGTAGAATCTATACCCCATTCACAGAAGATACCGAGACTGTG | |
| CCA-CTA | GGCCAGAGAGCCCTGCACTCAATTCTGAATGCTGC | |
| | GCAGCATTCAGAATTGAGTGCAGGGCTCTCTGGCCCACAGTC | 4105 |
| | TCGGTATCTTCTGTGAATGGGGTATAGATTCTACAATAAAACA | |
| | AACACAAAAGCCCTAGGTCAGTGTTAATGGAGATCA | |
| | CTATACCCCATTCACAG | 4106 |
| | CTGTGAATGGGGTATAG | 4107 |
| Alzheimer disease | TAACACTGACCTAGGGCTTTTGTGTTTGTTTTATTGTAGAATCT | 4108 |
| Glu120Asp | ATACCCCATTCACAGAAGATACCGAGACTGTGGGCCAGAGAG | |
| GAAg-GAT | CCCTGCACTCAATTCTGAATGCTGCCATCATGATC | |
| | GATCATGATGGCAGCATTCAGAATTGAGTGCAGGGCTCTCTG | 4109 |
| | GCCCACAGTCTCGGTATCTTCTGTGAATGGGGTATAGATTCT | |
| | ACAATAAAACAAACACAAAAGCCCTAGGTCAGTGTTA | |
| | TTCACAGAAGATACCGA | 4110 |
| | TCGGTATCTTCTGTGAA | 4111 |
| Alzheimer disease | TAACACTGACCTAGGGCTTTTGTGTTTGTTTTATTGTAGAATCT | 4112 |
| Glu120Asp | ATACCCCATTCACAGAAGATACCGAGACTGTGGGCCAGAGAG | |
| GAAg-GAC | CCCTGCACTCAATTCTGAATGCTGCCATCATGATC | |
| | GATCATGATGGCAGCATTCAGAATTGAGTGCAGGGCTCTCTG | 4113 |
| | GCCCACAGTCTCGGTATCTTCTGTGAATGGGGTATAGATTCT | |
| | ACAATAAAACAAACACAAAAGCCCTAGGTCAGTGTTA | |
| | TTCACAGAAGATACCGA | 4114 |
| | TCGGTATCTTCTGTGAA | 4115 |
| Alzheimer disease | ATTAACACTGACCTAGGGCTTTTGTGTTTGTTTTATTGTAGAAT | 4116 |
| Glu120Lys | CTATACCCCATTCACAGAAGATACCGAGACTGTGGGCCAGAG | |
| aGAA-AAA | AGCCCTGCACTCAATTCTGAATGCTGCCATCATGA | |
| | TCATGATGGCAGCATTCAGAATTGAGTGCAGGGCTCTCTGGC | 4117 |
| | CCACAGTCTCGGTATCTTCTGTGAATGGGGTATAGATTCTACA | |
| | ATAAAACAAACACAAAAGCCCTAGGTCAGTGTTAAT | |
| | CATTCACAGAAGATACC | 4118 |
| | GGTATCTTCTGTGAATG | 4119 |
| Alzheimer disease | GACCTAGGGCTTTTGTGTTTGTTTTATTGTAGAATCTATACCC | 4120 |
| Glu123Lys | CATTCACAGAAGATACCGAGACTGTGGGCCAGAGAGCCCTG | |
| cGAG-AAG | CACTCAATTCTGAATGCTGCCATCATGATCAGTGTCA | |
| | TGACACTGATCATGATGGCAGCATTCAGAATTGAGTGCAGGG | 4121 |
| | CTCTCTGGCCCACAGTCTCGGTATCTTCTGTGAATGGGGTAT | |
| | AGATTCTACAATAAAACAAACACAAAAGCCCTAGGTC | |
| | AAGATACCGAGACTGTG | 4122 |
| | CACAGTCTCGGTATCTT | 4123 |
| Alzheimer disease | TATACCCCATTCACAGAAGATACCGAGACTGTGGGCCAGAGA | 4124 |
| Asn135Asp | GCCCTGCACTCAATTCTGAATGCTGCCATCATGATCAGTGTC | |
| gAAT-GAT | ATTGTTGTCATGACTATCCTCCTGGTGGTTCTGTATA | |
| | TATACAGAACCACCAGGAGGATAGTCATGACAACAATGACAC | 4125 |
| | TGATCATGATGGCAGCATTCAGAATTGAGTGCAGGGCTCTCT | |
| | GGCCCACAGTCTCGGTATCTTCTGTGAATGGGGTATA | |
| | CAATTCTGAATGCTGCC | 4126 |
| | GGCAGCATTCAGAATTG | 4127 |
| Alzheimer disease | AGAAGATACCGAGACTGTGGGCCAGAGAGCCCTGCACTCAA | 4128 |
| Met139Ile | TTCTGAATGCTGCCATCATGATCAGTGTCATTGTTGTCATGAC | |
| ATGa-ATA | TATCCTCCTGGTGGTTCTGTATAAATACAGGTGCTAT | |
| | ATAGCACCTGTATTTATACAGAACCACCAGGAGGATAGTCAT | 4129 |
| | GACAACAATGACACTGATCATGATGGCAGCATTCAGAATTGA | |
| | GTGCAGGGCTCTCTGGCCCACAGTCTCGGTATCTTCT | |
| | GCCATCATGATCAGTGT | 4130 |
| | ACACTGATCATGATGGC | 4131 |
| Alzheimer disease | CAGAAGATACCGAGACTGTGGGCCAGAGAGCCCTGCACTCA | 4132 |
| Met139Lys | ATTCTGAATGCTGCCATCATGATCAGTGTCATTGTTGTCATGA | |
| ATG-AAG | CTATCCTCCTGGTGGTTCTGTATAAATACAGGTGCTA | |
| | TAGCACCTGTATTTATACAGAACCACCAGGAGGATAGTCATG | 4133 |
| | ACAACAATGACACTGATCATGATGGCAGCATTCAGAATTGAG | |
| | TGCAGGGCTCTCTGGCCCACAGTCTCGGTATCTTCTG | |
| | TGCCATCATGATCAGTG | 4134 |
| | CACTGATCATGATGGCA | 4135 |
| Alzheimer disease | CAGAAGATACCGAGACTGTGGGCCAGAGAGCCCTGCACTCA | 4136 |
| Met139Thr | ATTCTGAATGCTGCCATCATGATCAGTGTCATTGTTGTCATGA | |
| ATG-ACG | CTATCCTCCTGGTGGTTCTGTATAAATACAGGTGCTA | |
| | TAGCACCTGTATTTATACAGAACCACCAGGAGGATAGTCATG | 4137 |
| | ACAACAATGACACTGATCATGATGGCAGCATTCAGAATTGAG | |
| | TGCAGGGCTCTCTGGCCCACAGTCTCGGTATCTTCTG | |
| | TGCCATCATGATCAGTG | 4138 |
| | CACTGATCATGATGGCA | 4139 |
| Alzheimer disease | ACAGAAGATACCGAGACTGTGGGCCAGAGAGCCCTGCACTC | 4140 |
| Met139Val | AATTCTGAATGCTGCCATCATGATCAGTGTCATTGTTGTCATG | |
| cATG-GTG | ACTATCCTCCTGGTGGTTCTGTATAAATACAGGTGCT | |
| | AGCACCTGTATTTATACAGAACCACCAGGAGGATAGTCATGA | 4141 |
| | CAACAATGACACTGATCATGATGGCAGCATTCAGAATTGAGT | |
| | GCAGGGCTCTCTGGCCCACAGTCTCGGTATCTTCTGT | |
| | CTGCCATCATGATCAGT | 4142 |
| | ACTGATCATGATGGCAG | 4143 |
| Alzheimer disease | GAGACTGTGGGCCAGAGAGCCCTGCACTCAATTCTGAATGCT | 4144 |
| Ile143Phe | GCCATCATGATCAGTGTCATTGTTGTCATGACTATCCTCCTGG | |
| cATT-TTT | TGGTTCTGTATAAATACAGGTGCTATAAGGTGAGCA | |
| | TGCTCACCTTATAGCACCTGTATTTATACAGAACCACCAGGAG | 4145 |
| | GATAGTCATGACAACAATGACACTGATCATGATGGCAGCATT | |
| | CAGAATTGAGTGCAGGGCTCTCTGGCCCACAGTCTC | |
| | TCAGTGTCATTGTTGTC | 4146 |
| | GACAACAATGACACTGA | 4147 |
| Alzheimer disease | AGACTGTGGGCCAGAGAGCCCTGCACTCAATTCTGAATGCTG | 4148 |
| Ile143Thr | CCATCATGATCAGTGTCATTGTTGTCATGACTATCCTCCTGGT | |
| ATT-ACT | GGTTCTGTATAAATACAGGTGCTATAAGGTGAGCAT | |
| | ATGCTCACCTTATAGCACCTGTATTTATACAGAACCACCAGGA | 4149 |
| | GGATAGTCATGACAACAATGACACTGATCATGATGGCAGCAT | |
| | TCAGAATTGAGTGCAGGGCTCTCTGGCCCACAGTCT | |
| | CAGTGTCATTGTTGTCA | 4150 |
| | TGACAACAATGACACTG | 4151 |
| Alzheimer disease | CCAGAGAGCCCTGCACTCAATTCTGAATGCTGCCATCATGAT | 4152 |
| Met146Ile | CAGTGTCATTGTTGTCATGACTATCCTCCTGGTGGTTCTGTAT | |
| ATGa-ATA | AAATACAGGTGCTATAAGGTGAGCATGAGACACAGA | |
| | TCTGTGTCTCATGCTCACCTTATAGCACCTGTATTTATACAGA | 4153 |
| | ACCACCAGGAGGATAGTCATGACAACAATGACACTGATCATG | |
| | ATGGCAGCATTCAGAATTGAGTGCAGGGCTCTCTGG | |
| | GTTGTCATGACTATCCT | 4154 |
| | AGGATAGTCATGACAAC | 4155 |
| Alzheimer disease | CCAGAGAGCCCTGCACTCAATTCTGAATGCTGCCATCATGAT | 4156 |
| Met146Ile | CAGTGTCATTGTTGTCATGACTATCCTCCTGGTGGTTCTGTAT | |
| ATGa-ATC | AAATACAGGTGCTATAAGGTGAGCATGAGACACAGA | |
| | TCTGTGTCTCATGCTCACCTTATAGCACCTGTATTTATACAGA | 4157 |
| | ACCACCAGGAGGATAGTCATGACAACAATGACACTGATCATG | |
| | ATGGCAGCATTCAGAATTGAGTGCAGGGCTCTCTGG | |
| | GTTGTCATGACTATCCT | 4158 |
| | AGGATAGTCATGACAAC | 4159 |
| Alzheimer disease | GGCCAGAGAGCCCTGCACTCAATTCTGAATGCTGCCATCATG | 4160 |
| Met146Leu | ATCAGTGTCATTGTTGTCATGACTATCCTCCTGGTGGTTCTGT | |
| cATG-TTG | ATAAATACAGGTGCTATAAGGTGAGCATGAGACACA | |
| | TGTGTCTCATGCTCACCTTATAGCACCTGTATTTATACAGAAC | 4161 |
| | CACCAGGAGGATAGTCATGACAACAATGACACTGATCATGAT | |
| | GGCAGCATTCAGAATTGAGTGCAGGGCTCTCTGGCC | |
| | TTGTTGTCATGACTATC | 4162 |
| | GATAGTCATGACAACAA | 4163 |
| Alzheimer disease | GGCCAGAGAGCCCTGCACTCAATTCTGAATGCTGCCATCATG | 4164 |
| Met146Val | ATCAGTGTCATTGTTGTCATGACTATCCTCCTGGTGGTTCTGT | |
| cATG-GTG | ATAAATACAGGTGCTATAAGGTGAGCATGAGACACA | |
| | TGTGTCTCATGCTCACCTTATAGCACCTGTATTTATACAGAAC | 4165 |
| | CACCAGGAGGATAGTCATGACAACAATGACACTGATCATGAT | |
| | GGCAGCATTCAGAATTGAGTGCAGGGCTCTCTGGCC | |
| | TTGTTGTCATGACTATC | 4166 |
| | GATAGTCATGACAACAA | 4167 |
| Alzheimer disease | AGAGAGCCCTGCACTCAATTCTGAATGCTGCCATCATGATCA | 4168 |
| Thr147Ile | GTGTCATTGTTGTCATGACTATCCTCCTGGTGGTTCTGTATAA | |
| ACT-ATT | ATACAGGTGCTATAAGGTGAGCATGAGACACAGATC | |
| | GATCTGTGTCTCATGCTCACCTTATAGCACCTGTATTTATACA | 4169 |
| | GAACCACCAGGAGGATAGTCATGACAACAATGACACTGATCA | |
| | TGATGGCAGCATTCAGAATTGAGTGCAGGGCTCTCT | |
| | TGTCATGACTATCCTCC | 4170 |
| | GGAGGATAGTCATGACA | 4171 |
| Alzheimer disease | CTTTTTAAGGGTTGTGGGACCTGTTAATTATATTGAAATGCTTT | 4172 |
| His163Arg | CTTTTCTAGGTCATCCATGCCTGGCTTATTATATCATCTCTATT | |
| CAT-CGT | GTTGCTGTTCTTTTTTTCATTCATTTACTTGGG | |
| | CCCAAGTAAATGAATGAAAAAAAGAACAGCAACAATAGAGAT | 4173 |
| | GATATAATAAGCCAGGCATGGATGACCTAGAAAAGAAAGCAT | |
| | TTCAATATAATTAACAGGTCCCACAACCCTTAAAAAG | |
| | GGTCATCCATGCCTGGC | 4174 |
| | GCCAGGCATGGATGACC | 4175 |
| Alzheimer disease | ACTTTTTAAGGGTTGTGGGACCTGTTAATTATATTGAAATGCT | 4176 |
| His163Tyr | TTCTTTTCTAGGTCATCCATGCCTGGCTTATTATATCATCTCTA | |
| cCAT-TAT | TTGTTGCTGTTCTTTTTTTCATTCATTTACTTGG | |
| | CCAAGTAAATGAATGAAAAAAAGAACAGCAACAATAGAGATGA | 4177 |
| | TATAATAAGCCAGGCATGGATGACCTAGAAAAGAAAGCATTTC | |
| | AATATAATTAACAGGTCCCACAACCCTTAAAAAGT | |
| | AGGTCATCCATGCCTGG | 4178 |
| | CCAGGCATGGATGACCT | 4179 |
| Alzheimer disease | AGGGTTGTGGGACCTGTTAATTATATTGAAATGCTTTCTTTTC | 4180 |
| Trp165Cys | TAGGTCATCCATGCCTGGCTTATTATATCATCTCTATTGTTGCT | |
| TGGc-TGC | GTTCTTTTTTTCATTCATTTACTTGGGGTAAGTT | |
| | AACTTACCCCAAGTAAATGAATGAAAAAAAGAACAGCAACAAT | 4181 |
| | AGAGATGATATAATAAGCCAGGCATGGATGACCTAGAAAAGA | |
| | AAGCATTTCAATATAATTAACAGGTCCCACAACCCT | |
| | CATGCCTGGCTTATTAT | 4182 |
| | ATAATAAGCCAGGCATG | 4183 |
| Alzheimer disease | ACCTGTTAATTATATTGAAATGCTTTCTTTTCTAGGTCATCCAT | 4184 |
| Ser169Leu | GCCTGGCTTATTATATCATCTCTATTGTTGCTGTTCTTTTTTTC | |
| TCA-TTA | ATTCATTTACTTGGGGTAAGTTGTGAAATTTTT | |
| | AAAAATTTCACAACTTACCCCAAGTAAATGAATGAAAAAAAGA | 4185 |
| | ACAGCAACAATAGAGATGATATAATAAGCCAGGCATGGATGA | |
| | CCTAGAAAAGAAAGCATTTCAATATAATTAACAGGT | |
| | TATTATATCATCTCTAT | 4186 |
| | ATAGAGATGATATAATA | 4187 |
| Alzheimer disease | TAATTATATTGAAATGCTTTCTTTTCTAGGTCATCCATGCCTGG | 4188 |
| Leu171Pro | CTTATTATATCATCTCTATTGTTGCTGTTCTTTTTTTCATTCATT | |
| CTA-CCA | TACTTGGGGTAAGTTGTGAAATTTTTGGTCTG | |
| | CAGACCAAAAATTTCACAACTTACCCCAAGTAAATGAATGAAA | 4189 |
| | AAAAGAACAGCAACAATAGAGATGATATAATAAGCCAGGCAT | |
| | GGATGACCTAGAAAAGAAAGCATTTCAATATAATTA | |
| | ATCATCTCTATTGTTGC | 4190 |
| | GCAACAATAGAGATGAT | 4191 |
| Alzheimer disease | TATTGAAATGCTTTCTTTTCTAGGTCATCCATGCCTGGCTTATT | 4192 |
| Leu173Trp | ATATCATCTCTATTGTTGCTGTTCTTTTTTTCATTCATTTACTTG | |
| TTG-TGG | GGGTAAGTTGTGAAATTTTTGGTCTGTCTTTC | |
| | GAAAGACAGACCAAAAATTTCACAACTTACCCCAAGTAAATGA | 4193 |
| | ATGAAAAAAAGAACAGCAACAATAGAGATGATATAATAAGCCA | |
| | GGCATGGATGACCTAGAAAAGAAAGCATTTCAATA | |
| | TCTATTGTTGCTGTTCT | 4194 |
| | AGAACAGCAACAATAGA | 4195 |
| Alzheimer disease | TATAACGTTGCTGTGGACTACATTACTGTTGCACTCCTGATCT | 4196 |
| Gly209Arg | GGAATTTTGGTGTGGTGGGAATGATTTCCATTCACTGGAAAG | |
| gGGA-AGA | GTCCACTTCGACTCCAGCAGGCATATCTCATTATGA | |
| | TCATAATGAGATATGCCTGCTGGAGTCGAAGTGGACCTTTCC | 4197 |
| | AGTGAATGGAAATCATTCCCACCACACCAAAATTCCAGATCAG | |
| | GAGTGCAACAGTAATGTAGTCCACAGCAACGTTATA | |
| | GTGTGGTGGGAATGATT | 4198 |
| | AATCATTCCCACCACAC | 4199 |
| Alzheimer disease | ATAACGTTGCTGTGGACTACATTACTGTTGCACTCCTGATCTG | 4200 |
| Gly209Val | GAATTTTGGTGTGGTGGGAATGATTTCCATTCACTGGAAAGG | |
| GGA-GTA | TCCACTTCGACTCCAGCAGGCATATCTCATTATGAT | |
| | ATCATAATGAGATATGCCTGCTGGAGTCGAAGTGGACCTTTC | 4201 |
| | CAGTGAATGGAAATCATTCCCACCACACCAAAATTCCAGATCA | |
| | GGAGTGCAACAGTAATGTAGTCCACAGCAACGTTAT | |
| | TGTGGTGGGAATGATTT | 4202 |
| | AAATCATTCCCACCACA | 4203 |
| Alzheimer disease | TGGACTACATTACTGTTGCACTCCTGATCTGGAATTTTGGTGT | 4204 |
| Ile213Thr | GGTGGGAATGATTTCCATTCACTGGAAAGGTCCACTTCGACT | |
| ATT-ACT | CCAGCAGGCATATCTCATTATGATTAGTGCCCTCAT | |
| | ATGAGGGCACTAATCATAATGAGATATGCCTGCTGGAGTCGA | 4205 |
| | AGTGGACCTTTCCAGTGAATGGAAATCATTCCCACCACACCA | |
| | AAATTCCAGATCAGGAGTGCAACAGTAATGTAGTCCA | |
| | GATTTCCATTCACTGGA | 4206 |
| | TCCAGTGAATGGAAATC | 4207 |
| Alzheimer disease | CACTCCTGATCTGGAATTTTGGTGTGGTGGGAATGATTTCCAT | 4208 |
| Leu219Pro | TCACTGGAAAGGTCCACTTCGACTCCAGCAGGCATATCTCAT | |
| CTT-CCT | TATGATTAGTGCCCTCATGGCCCTGGTGTTTATCAA | |
| | TTGATAAACACCAGGGCCATGAGGGCACTAATCATAATGAGA | 4209 |
| | TATGCCTGCTGGAGTCGAAGTGGACCTTTCCAGTGAATGGAA | |
| | ATCATTCCCACCACACCAAAATTCCAGATCAGGAGTG | |
| | AGGTCCACTTCGACTCC | 4210 |
| | GGAGTCGAAGTGGACCT | 4211 |
| Alzheimer disease | ATTTCCATTCACTGGAAAGGTCCACTTCGACTCCAGCAGGCA | 4212 |
| Ala231Thr | TATCTCATTATGATTAGTGCCCTCATGGCCCTGGTGTTTATCA | |
| tGCC-ACC | AGTACCTCCCTGAATGGACTGCGTGGCTCATCTTGG | |
| | CCAAGATGAGCCACGCAGTCCATTCAGGGAGGTACTTGATAA | 4213 |
| | ACACCAGGGCCATGAGGGCACTAATCATAATGAGATATGCCT | |
| | GCTGGAGTCGAAGTGGACCTTTCCAGTGAATGGAAAT | |
| | TGATTAGTGCCCTCATG | 4214 |
| | CATGAGGGCACTAATCA | 4215 |
| Alzheimer disease | TTTCCATTCACTGGAAAGGTCCACTTCGACTCCAGCAGGCAT | 4216 |
| Ala231Val | ATCTCATTATGATTAGTGCCCTCATGGCCCTGGTGTTTATCAA | |
| GCC-GTC | GTACCTCCCTGAATGGACTGCGTGGCTCATCTTGGC | |
| | GCCAAGATGAGCCACGCAGTCCATTCAGGGAGGTACTTGATA | 4217 |
| | AACACCAGGGCCATGAGGGCACTAATCATAATGAGATATGCC | |
| | TGCTGGAGTCGAAGTGGACCTTTCCAGTGAATGGAAA | |
| | GATTAGTGCCCTCATGG | 4218 |
| | CCATGAGGGCACTAATC | 4219 |
| Alzheimer disease | TTCACTGGAAAGGTCCACTTCGACTCCAGCAGGCATATCTCA | 4220 |
| Met233Thr | TTATGATTAGTGCCCTCATGGCCCTGGTGTTTATCAAGTACCT | |
| ATG-ACG | CCCTGAATGGACTGCGTGGCTCATCTTGGCTGTGAT | |
| | ATCACAGCCAAGATGAGCCACGCAGTCCATTCAGGGAGGTAC | 4221 |
| | TTGATAAACACCAGGGCCATGAGGGCACTAATCATAATGAGA | |
| | TATGCCTGCTGGAGTCGAAGTGGACCTTTCCAGTGAA | |
| | TGCCCTCATGGCCCTGG | 4222 |
| | CCAGGGCCATGAGGGCA | 4223 |
| Alzheimer disease | GGAAAGGTCCACTTCGACTCCAGCAGGCATATCTCATTATGA | 4224 |
| Leu235Pro | TTAGTGCCCTCATGGCCCTGGTGTTTATCAAGTACCTCCCTG | |
| CTG-CCG | AATGGACTGCGTGGCTCATCTTGGCTGTGATTTCAGT | |
| | ACTGAAATCACAGCCAAGATGAGCCACGCAGTCCATTCAGGG | 4225 |
| | AGGTACTTGATAAACACCAGGGCCATGAGGGCACTAATCATA | |
| | ATGAGATATGCCTGCTGGAGTCGAAGTGGACCTTTCC | |
| | CATGGCCCTGGTGTTTA | 4226 |
| | TAAACACCAGGGCCATG | 4227 |
| Alzheimer disease | TCATTATGATTAGTGCCCTCATGGCCCTGGTGTTTATCAAGTA | 4228 |
| Ala246Glu | CCTCCCTGAATGGACTGCGTGGCTCATCTTGGCTGTGATTTC | |
| GCG-GAG | AGTATATGGTAAAACCCAAGACTGATAATTTGTTTG | |
| | CAAACAAATTATCAGTCTTGGGTTTTACCATATACTGAAATCA | 4229 |
| | CAGCCAAGATGAGCCACGCAGTCCATTCAGGGAGGTACTTGA | |
| | TAAACACCAGGGCCATGAGGGCACTAATCATAATGA | |
| | ATGGACTGCGTGGCTCA | 4230 |
| | TGAGCCACGCAGTCCAT | 4231 |
| Alzheimer disease | GTGCCCTCATGGCCCTGGTGTTTATCAAGTACCTCCCTGAAT | 4232 |
| Leu250Ser | GGACTGCGTGGCTCATCTTGGCTGTGATTTCAGTATATGGTA | |
| TTG-TCG | AAACCCAAGACTGATAATTTGTTTGTCACAGGAATGC | |
| | GCATTCCTGTGACAAACAAATTATCAGTCTTGGGTTTTACCAT | 4233 |
| | ATACTGAAATCACAGCCAAGATGAGCCACGCAGTCCATTCAG | |
| | GGAGGTACTTGATAAACACCAGGGCCATGAGGGCAC | |
| | GCTCATCTTGGCTGTGA | 4234 |
| | TCACAGCCAAGATGAGC | 4235 |
| Alzheimer disease | AGTTTAGCCCATACATTTTATTAGATGTCTTTTATGTTTTTCTTT | 4236 |
| Ala260Val | TTCTAGATTTAGTGGCTGTTTTGTGTCCGAAAGGTCCACTTCG | |
| GCT-GTT | TATGCTGGTTGAAACAGCTCAGGAGAGAAATGA | |
| | TCATTTCTCTCCTGAGCTGTTTCAACCAGCATACGAAGTGGAC | 4237 |
| | CTTTCGGACACAAAACAGCCACTAAATCTAGAAAAAGAAAAAC | |
| | ATAAAAGACATCTAATAAAATGTATGGGCTAAACT | |
| | TTTAGTGGCTGTTTTGT | 4238 |
| | ACAAAACAGCCACTAAA | 4239 |
| Alzheimer disease | CCCATACATTTTATTAGATGTCTTTTATGTTTTTCTTTTTCTAGA | 4240 |
| Leu262Phe | TTTAGTGGCTGTTTTGTGTCCGAAAGGTCCACTTCGTATGCTG | |
| TTGt-TTC | GTTGAAACAGCTCAGGAGAGAAATGAAACGCTT | |
| | AAGCGTTTCATTTCTCTCCTGAGCTGTTTCAACCAGCATACGA | 4241 |
| | AGTGGACCTTTCGGACACAAAACAGCCACTAAATCTAGAAAA | |
| | AGAAAAACATAAAAGACATCTAATAAAATGTATGGG | |
| | GCTGTTTTGTGTCCGAA | 4242 |
| | TTCGGACACAAAACAGC | 4243 |
| Alzheimer disease | CCATACATTTTATTAGATGTCTTTTATGTTTTTCTTTTTCTAGAT | 4244 |
| Cys263Arg | TTAGTGGCTGTTTTGTGTCCGAAAGGTCCACTTCGTATGCTG | |
| gTGT-CGT | GTTGAAACAGCTCAGGAGAGAAATGAAACGCTTT | |
| | AAAGCGTTTCATTTCTCTCCTGAGCTGTTTCAACCAGCATACG | 4245 |
| | AAGTGGACCTTTCGGACACAAAACAGCCACTAAATCTAGAAA | |
| | AAGAAAAACATAAAAGACATCTAATAAAATGTATGG | |
| | CTGTTTTGTGTCCGAAA | 4246 |
| | TTTCGGACACAAAACAG | 4247 |
| Alzheimer disease | ACATTTTATTAGATGTCTTTTATGTTTTTCTTTTTCTAGATTTAG | 4248 |
| Pro264Leu | TGGCTGTTTTGTGTCCGAAAGGTCCACTTCGTATGCTGGTTG | |
| CCG-CTG | AAACAGCTCAGGAGAGAAATGAAACGCTTTTTCC | |
| | GGAAAAAGCGTTTCATTTCTCTCCTGAGCTGTTTCAACCAGCA | 4249 |
| | TACGAAGTGGACCTTTCGGACACAAAACAGCCACTAAATCTA | |
| | GAAAAAGAAAAACATAAAAGACATCTAATAAAATGT | |
| | TTTGTGTCCGAAAGGTC | 4250 |
| | GACCTTTCGGACACAAA | 4251 |
| Alzheimer disease | GTCTTTTATGTTTTTCTTTTTCTAGATTTAGTGGCTGTTTTGTG | 4252 |
| Arg269Gly | TCCGAAAGGTCCACTTCGTATGCTGGTTGAAACAGCTCAGGA | |
| tCGT-GGT | GAGAAATGAAACGCTTTTTCCAGCTCTCATTTACT | |
| | AGTAAATGAGAGCTGGAAAAAGCGTTTCATTTCTCTCCTGAGC | 4253 |
| | TGTTTCAACCAGCATACGAAGTGGACCTTTCGGACACAAAAC | |
| | AGCCACTAAATCTAGAAAAAGAAAAACATAAAAGAC | |
| | GTCCACTTCGTATGCTG | 4254 |
| | CAGCATACGAAGTGGAC | 4255 |
| Alzheimer disease | TCTTTTATGTTTTTCTTTTTCTAGATTTAGTGGCTGTTTTGTGTC | 4256 |
| Arg269His | CGAAAGGTCCACTTCGTATGCTGGTTGAAACAGCTCAGGAGA | |
| CGT-CAT | GAAATGAAACGCTTTTTCCAGCTCTCATTTACTC | |
| | GAGTAAATGAGAGCTGGAAAAAGCGTTTCATTTCTCTCCTGA | 4257 |
| | GCTGTTTCAACCAGCATACGAAGTGGACCTTTCGGACACAAA | |
| | ACAGCCACTAAATCTAGAAAAAGAAAAACATAAAAGA | |
| | TCCACTTCGTATGCTGG | 4258 |
| | CCAGCATACGAAGTGGA | 4259 |
| Alzheimer disease | TAGTGGCTGTTTTGTGTCCGAAAGGTCCACTTCGTATGCTGG | 4260 |
| Arg278Thr | TTGAAACAGCTCAGGAGAGAAATGAAACGCTTTTTCCAGCTCT | |
| AGA-ACA | CATTTACTCCTGTAAGTATTTGAGAATGATATTGAA | |
| | TTCAATATCATTCTCAAATACTTACAGGAGTAAATGAGAGCTG | 4261 |
| | GAAAAAGCGTTTCATTTCTCTCCTGAGCTGTTTCAACCAGCAT | |
| | ACGAAGTGGACCTTTCGGACACAAAACAGCCACTA | |
| | TCAGGAGAGAAATGAAA | 4262 |
| | TTTCATTTCTCTCCTGA | 4263 |
| Alzheimer disease | CTGTTTTGTGTCCGAAAGGTCCACTTCGTATGCTGGTTGAAAC | 4264 |
| Glu280Ala | AGCTCAGGAGAGAAATGAAACGCTTTTTCCAGCTCTCATTTAC | |
| GAA-GCA | TCCTGTAAGTATTTGAGAATGATATTGAATTAGTA | |
| | TACTAATTCAATATCATTCTCAAATACTTACAGGAGTAAATGAG | 4265 |
| | AGCTGGAAAAAGCGTTTCATTTCTCTCCTGAGCTGTTTCAACC | |
| | AGCATACGAAGTGGACCTTTCGGACACAAAACAG | |
| | GAGAAATGAAACGCTTT | 4266 |
| | AAAGCGTTTCATTTCTC | 4267 |
| Alzheimer disease | CTGTTTTGTGTCCGAAAGGTCCACTTCGTATGCTGGTTGAAAC | 4268 |
| Glu280Gly | AGCTCAGGAGAGAAATGAAACGCTTTTTCCAGCTCTCATTTAC | |
| GAA-GGA | TCCTGTAAGTATTTGAGAATGATATTGAATTAGTA | |
| | TACTAATTCAATATCATTCTCAAATACTTACAGGAGTAAATGAG | 4269 |
| | AGCTGGAAAAAGCGTTTCATTTCTCTCCTGAGCTGTTTCAACC | |
| | AGCATACGAAGTGGACCTTTCGGACACAAAACAG | |
| | GAGAAATGAAACGCTTT | 4270 |
| | AAAGCGTTTCATTTCTC | 4271 |
| Alzheimer disease | TGTGTCCGAAAGGTCCACTTCGTATGCTGGTTGAAACAGCTC | 4272 |
| Leu282Arg | AGGAGAGAAATGAAACGCTTTTTCCAGCTCTCATTTACTCCTG | |
| CTT-CGT | TAAGTATTTGAGAATGATATTGAATTAGTAATCAGT | |
| | ACTGATTACTAATTCAATATCATTCTCAAATACTTACAGGAGTA | 4273 |
| | AATGAGAGCTGGAAAAAGCGTTTCATTTCTCTCCTGAGCTGTT | |
| | TCAACCAGCATACGAAGTGGACCTTTCGGACACA | |
| | TGAAACGCTTTTTCCAG | 4274 |
| | CTGGAAAAAGCGTTTCA | 4275 |
| Alzheimer disease | AAGGTCCACTTCGTATGCTGGTTGAAACAGCTCAGGAGAGAA | 4276 |
| Ala285Val | ATGAAACGCTTTTTCCAGCTCTCATTTACTCCTGTAAGTATTTG | |
| GCT-GTT | AGAATGATATTGAATTAGTAATCAGTGTAGAATTT | |
| | AAATTCTACACTGATTACTAATTCAATATCATTCTCAAATACTT | 4277 |
| | ACAGGAGTAAATGAGAGCTGGAAAAAGCGTTTCATTTCTCTC | |
| | CTGAGCTGTTTCAACCAGCATACGAAGTGGACCTT | |
| | TTTTCCAGCTCTCATTT | 4278 |
| | AAATGAGAGCTGGAAAA | 4279 |
| Alzheimer disease | GGTCCACTTCGTATGCTGGTTGAAACAGCTCAGGAGAGAAAT | 4280 |
| Leu286Val | GAAACGCTTTTTCCAGCTCTCATTTACTCCTGTAAGTATTTGA | |
| tCTC-GTC | GAATGATATTGAATTAGTAATCAGTGTAGAATTTAT | |
| | ATAAATTCTACACTGATTACTAATTCAATATCATTCTCAAATAC | 4281 |
| | TTACAGGAGTAAATGAGAGCTGGAAAAAGCGTTTCATTTCTCT | |
| | CCTGAGCTGTTTCAACCAGCATACGAAGTGGACC | |
| | TTCCAGCTCTCATTTAC | 4282 |
| | GTAAATGAGAGCTGGAA | 4283 |
| Alzheimer disease | GTGACCAACTTTTTAATATTTGTAACCTTTCCTTTTTAGGGGGA | 4284 |
| Gly384Ala | GTAAAACTTGGATTGGGAGATTTCATTTTCTACAGTGTTCTGG | |
| GGA-GCA | TTGGTAAAGCCTCAGCAACAGCCAGTGGAGACTG | |
| | CAGTCTCCACTGGCTGTTGCTGAGGCTTTACCAACCAGAACA | 4285 |
| | CTGTAGAAAATGAAATCTCCCAATCCAAGTTTTACTCCCCCTA | |
| | AAAAGGAAAGGTTACAAATATTAAAAAGTTGGTCAC | |
| | TGGATTGGGAGATTTCA | 4286 |
| | TGAAATCTCCCAATCCA | 4287 |
| Alzheimer disease | TTTGTAACCTTTCCTTTTTAGGGGGAGTAAAACTTGGATTGGG | 4288 |
| Ser390Ile | AGATTTCATTTTCTACAGTGTTCTGGTTGGTAAAGCCTCAGCA | |
| AGT-ATT | ACAGCCAGTGGAGACTGGAACACAACCATAGCCTG | |
| | CAGGCTATGGTTGTGTTCCAGTCTCCACTGGCTGTTGCTGAG | 4289 |
| | GCTTTACCAACCAGAACACTGTAGAAAATGAAATCTCCCAATC | |
| | CAAGTTTTACTCCCCCTAAAAAGGAAAGGTTACAAA | |
| | TTTCTACAGTGTTCTGG | 4290 |
| | CCAGAACACTGTAGAAA | 4291 |
| Alzheimer disease | AACCTTTCCTTTTTAGGGGGAGTAAAACTTGGATTGGGAGATT | 4292 |
| Leu392Val | TCATTTTCTACAGTGTTCTGGTTGGTAAAGCCTCAGCAACAGC | |
| tCTG-GTG | CAGTGGAGACTGGAACACAACCATAGCCTGTTTCG | |
| | CGAAACAGGCTATGGTTGTGTTCCAGTCTCCACTGGCTGTTG | 4293 |
| | CTGAGGCTTTACCAACCAGAACACTGTAGAAAATGAAATCTCC | |
| | CAATCCAAGTTTTACTCCCCCTAAAAAGGAAAGGTT | |
| | ACAGTGTTCTGGTTGGT | 4294 |
| | ACCAACCAGAACACTGT | 4295 |
| Alzheimer disease | ATTTCATTTTCTACAGTGTTCTGGTTGGTAAAGCCTCAGCAAC | 4296 |
| Asn405Ser | AGCCAGTGGAGACTGGAACACAACCATAGCCTGTTTCGTAGC | |
| AAC-AGC | CATATTAATTGTAAGTATACACTAATAAGAATGTGT | |
| | ACACATTCTTATTAGTGTATACTTACAATTAATATGGCTACGAA | 4297 |
| | ACAGGCTATGGTTGTGTTCCAGTCTCCACTGGCTGTTGCTGA | |
| | GGCTTTACCAACCAGAACACTGTAGAAAATGAAAT | |
| | AGACTGGAACACAACCA | 4298 |
| | TGGTTGTGTTCCAGTCT | 4299 |
| Alzheimer disease | TACAGTGTTCTGGTTGGTAAAGCCTCAGCAACAGCCAGTGGA | 4300 |
| Ala409Thr | GACTGGAACACAACCATAGCCTGTTTCGTAGCCATATTAATTG | |
| aGCC-ACC | TAAGTATACACTAATAAGAATGTGTCAGAGCTCTTA | |
| | TAAGAGCTCTGACACATTCTTATTAGTGTATACTTACAATTAAT | 4301 |
| | ATGGCTACGAAACAGGCTATGGTTGTGTTCCAGTCTCCACTG | |
| | GCTGTTGCTGAGGCTTTACCAACCAGAACACTGTA | |
| | CAACCATAGCCTGTTTC | 4302 |
| | GAAACAGGCTATGGTTG | 4303 |
| Alzheimer disease | GTGTTCTGGTTGGTAAAGCCTCAGCAACAGCCAGTGGAGACT | 4304 |
| Cys410Tyr | GGAACACAACCATAGCCTGTTTCGTAGCCATATTAATTGTAAG | |
| TGT-TAT | TATACACTAATAAGAATGTGTCAGAGCTCTTAATGT | |
| | ACATTAAGAGCTCTGACACATTCTTATTAGTGTATACTTACAAT | 4305 |
| | TAATATGGCTACGAAACAGGCTATGGTTGTGTTCCAGTCTCCA | |
| | CTGGCTGTTGCTGAGGCTTTACCAACCAGAACAC | |
| | CATAGCCTGTTTCGTAG | 4306 |
| | CTACGAAACAGGCTATG | 4307 |
| Alzheimer disease | TGTGAATGTGTGTCTTTCCCATCTTCTCCACAGGGTTTGTGCC | 4308 |
| Ala426Pro | TTACATTATTACTCCTTGCCATTTTCAAGAAAGCATTGCCAGC | |
| tGCC-CCC | TCTTCCAATCTCCATCACCTTTGGGCTTGTTTTCT | |
| | AGAAAACAAGCCCAAAGGTGATGGAGATTGGAAGAGCTGGC | 4309 |
| | AATGCTTTCTTGAAAATGGCAAGGAGTAATAATGTAAGGCACA | |
| | AACCCTGTGGAGAAGATGGGAAAGACACACATTCACA | |
| | TACTCCTTGCCATTTTC | 4310 |
| | GAAAATGGCAAGGAGTA | 4311 |
| Alzheimer disease | AGGGTTTGTGCCTTACATTATTACTCCTTGCCATTTTCAAGAA | 4312 |
| Pro436Gln | AGCATTGCCAGCTCTTCCAATCTCCATCACCTTTGGGCTTGTT | |
| CCA-CAA | TTCTACTTTGCCACAGATTATCTTGTACAGCCTTT | |
| | AAAGGCTGTACAAGATAATCTGTGGCAAAGTAGAAAACAAGC | 4313 |
| | CCAAAGGTGATGGAGATTGGAAGAGCTGGCAATGCTTTCTTG | |
| | AAAATGGCAAGGAGTAATAATGTAAGGCACAAACCCT | |
| | AGCTCTTCCAATCTCCA | 4314 |
| | TGGAGATTGGAAGAGCT | 4315 |
| Alzheimer disease | CAGGGTTTGTGCCTTACATTATTACTCCTTGCCATTTTCAAGA | 4316 |
| Pro436Ser | AAGCATTGCCAGCTCTTCCAATCTCCATCACCTTTGGGCTTGT | |
| tCCA-TCA | TTTCTACTTTGCCACAGATTATCTTGTACAGCCTT | |
| | AAGGCTGTACAAGATAATCTGTGGCAAAGTAGAAAACAAGCC | 4317 |
| | CAAAGGTGATGGAGATTGGAAGAGCTGGCAATGCTTTCTTGA | |
| | AAATGGCAAGGAGTAATAATGTAAGGCACAAACCCTG | |
| | CAGCTCTTCCAATCTCC | 4318 |
| | GGAGATTGGAAGAGCTG | 4319 |

### EXAMPLE 26

### Alzheimer's Disease—Presenilin—2 (PSEN2)

The attached table discloses the correcting oligonucleotide base sequences for the PSEN2 oligonucleotides of the invention.

TABLE 28
PSEN2 Mutations And Genome-Correcting Oligos

| | |
|---|---|
| Clinical Phenotype & | SEQ ID |

| | | |
|---|---|---|
| Mutation | Correcting Oligos | NO: |

| | | |
|---|---|---|
| Alzheimer disease | GATGTGGTTTCCCACAGAGAAGCCAGGAGAACGAGGAGGAC | 4320 |
| Arg62His | GGTGAGGAGGACCCTGACCGCTATGTCTGTAGTGGGGTTCC | |
| CGC-CAC | CGGGCGGCCGCCAGGCCTGGAGGAAGAGCTGACCCTCAA | |
| | TTGAGGGTCAGCTCTTCCTCCAGGCCTGGCGGCCGCCCGGG | 4321 |
| | AACCCCACTACAGACATAGCGGTCAGGGTCCTCCTCACCGTC | |
| | CTCCTCGTTCTCCTGGCTTCTCTGTGGGAAACCACATC | |
| | CCCTGACCGCTATGTCT | 4322 |
| | AGACATAGCGGTCAGGG | 4323 |
| Alzheimer disease | GCCTCGAGGAGCAGTCAGGGCCGGGAGCATCAGCCCTTTGC | 4324 |
| Thr122Pro | CTTCTCCCTCAGCATCTACACGACATTCACTGAGGACACACC | |
| cACG-CCG | CTCGGTGGGCCAGCGCCTCCTCAACTCCGTGCTGAACA | |
| | TGTTCAGCACGGAGTTGAGGAGGCGCTGGCCCACCGAGGGT | 4325 |
| | GTGTCCTCAGTGAATGTCGTGTAGATGCTGAGGGAGAAGGCA | |
| | AAGGGCTGATGCTCCCGGCCCTGACTGCTCCTCGAGGC | |
| | GCATCTACACGACATTC | 4326 |
| | GAATGTCGTGTAGATGC | 4327 |
| Alzheimer disease | ACACGCCATTCACTGAGGACACACCCTCGGTGGGCCAGCGC | 4328 |
| Asn141Ile | CTCCTCAACTCCGTGCTGAACACCCTCATCATGATCAGCGTC | |
| AAC-ATC | ATCGTGGTTATGACCATCTTCTTGGTGGTGCTCTACAA | |
| | TTGTAGAGCACCACCAAGAAGATGGTCATAACCACGATGACG | 4329 |
| | CTGATCATGATGAGGGTGTTCAGCACGGAGTTGAGGAGGCG | |
| | CTGGCCCACCGAGGGTGTGTCCTCAGTGAATGGCGTGT | |
| | CGTGCTGAACACCCTCA | 4330 |
| | TGAGGGTGTTCAGCACG | 4331 |
| Alzheimer disease | CCACTGGAAGGGCCCTCTGGTGCTGCAGCAGGCCTACCTCAT | 4332 |
| Met239Ile | CATGATCAGTGCGCTCATGGCCCTAGTGTTCATCAAGTACCTC | |
| ATGg-ATA | CCAGAGTGGTCCGCGTGGGTCATCCTGGGCGCCATC | |
| | GATGGCGCCCAGGATGACCCACGCGGACCACTCTGGGAGGT | 4333 |
| | ACTTGATGAACACTAGGGCCATGAGCGCACTGATCATGATGA | |
| | GGTAGGCCTGCTGCAGCACCAGAGGGCCCTTCCAGTGG | |
| | GCGCTCATGGCCCTAGT | 4334 |
| | ACTAGGGCCATGAGCGC | 4335 |
| Alzheimer disease | ATCCACTGGAAGGGCCCTCTGGTGCTGCAGCAGGCCTACCTC | 4336 |
| Met239Val | ATCATGATCAGTGCGCTCATGGCCCTAGTGTTCATCAAGTAC | |
| cATG-GTG | CTCCCAGAGTGGTCCGCGTGGGTCATCCTGGGCGCCA | |
| | TGGCGCCCAGGATGACCCACGCGGACCACTCTGGGAGGTAC | 4337 |
| | TTGATGAACACTAGGGCCATGAGCGCACTGATCATGATGAGG | |
| | TAGGCCTGCTGCAGCACCAGAGGGCCCTTCCAGTGGAT | |
| | GTGCGCTCATGGCCCTA | 4338 |
| | TAGGGCCATGAGCGCAC | 4339 |

### EXAMPLE 26

### Engineering Herbicide Resistant Plants

Chemical weed control is an important tool of modern agriculture and many herbicides have been developed for this purpose. Their use has resulted in substantial increases in the yields of many crops, including, for example, maize, soybeans, and cotton. Thus while the use of fertilizers and new high-yielding crop varieties have contributed greatly to the “green revolution,” chemical weed control has also been at the forefront of technological achievement.

Herbicides having broad-spectrum activity are particularly useful because they obviate the need for multiple herbicides targeting different classes of weeds. The problem with such herbicides is that they typically also affect crops which are exposed to the herbicide. One way to overcome this is to generate plants which are resistant to one or more broad-spectrum herbicides. Such herbicide-tolerant plants may reduce the need for tillage to control weeds, thereby effectively reducing soil erosion and can reduce the quantity and number of different herbicides applied in the field.

Common herbicides used, for example, include those that inhibit the enzyme 5-enolpyruvyl-3-phosphoshikimic acid synthase (EPSPS), for example N-phosphonomethyl-glycine (e.g. glyphosate), those that inhibit acetolactate synthase (ALS) activity, for example the sulfonylureas and related herbicides, and those that inhibit dihydropteroate synthase, for example methyl[(4-amino-phenyl)sulfonyl]carbamate (e.g. Asulam). Herbicide-tolerant plants can be produced by several methods, including, for example, introducing into the genome of the plant the ability to degrade the herbicide, the capacity to produce a higher level of the targeted enzyme, and/or expressing an herbicide-tolerant allele of the enzyme.

The attached tables disclose exemplary oligonucleotides base sequences which can be used to generate site-specific mutations in plant genes that confer herbicide resistance.

TABLE 28
Genome-Altering Oligos Conferring Glyphosate Resistance

Phenotype, Gene,

| | |
|---|---|
| Plant & Targeted | SEQ ID |

| | | |
|---|---|---|
| Alteration | Altering Oligos | NO: |

| | | |
|---|---|---|
| Glyphosate Resistance | AAGCGTCGGAGATTGTACTTCAACCCATTAGAGAAATCTCCGGTC | 4341 |
| EPSPS | TTATTAAGCTTCCTGCCTCCAAGTCTCTATCAAATCGGATCCTGC | |
| Arabidopsis thaliana | TTCTCGCTGCTCTGTCTGAGGTATATATCAC | |
| Gly97Ala | GTGATATATAGCTCAGACAGAGCAGCGAGAAGCAGGATCCGATT | 4342 |
| GGC-GCC | TGATAGAGACTTGGAGGCAGGAAGCTTAATAAGACCGGAGATTT | |
| | CTCTAATGGGTTGAAGTACAATCTCCGACGCTT | |
| | GCTTCCTGCCTCCAAGT | 4343 |
| | ACTTGGAGGCAGGAAGC | 4344 |
| Glyphosate Resistance | AAGCTTCAGAGATTGTGCTTCAACCAATCAGAGAAATCTCGGGTC | 4345 |
| EPSPS | TCATTAAGCTACCCGCATCCAAATCTCTCTCCAATCGGATCCTCC | |
| Brassica napus | TTCTTGCCGCTCTATCTGAGGTACATATACT | |
| Gly93Ala | AGTATATGTACCTCAGATAGAGCGGCAAGAAGGAGGATCCGATT | 4346 |
| GGA-GCA | GGAGAGAGATTTGGATGCGGGTAGCTTAATGAGACCCGAGATTT | |
| | CTCTGATTGGTTGAAGCACAATCTCTGAAGCTT | |
| | GCTACCCGCATCCAAAT | 4347 |
| | ATTTGGATGCGGGTAGC | 4348 |
| Glyphosate Resistance | AGCCCAACGAGATTGTGCTGCAACCCATCAAAGATATATCAGGC | 4349 |
| EPSPS 1 | ACTGTTAAATTGCCTGCTTCTAAATCCCTTTCCAATCGTATTCTCC | |
| Nicotiana tabacum | TTCTTGCTGCCCTTTCTAAGGGAAGGACTGT | |
| Gly95Ala | ACAGTCCTTCCCTTAGAAAGGGCAGCAAGAAGGAGAATACGATT | 4350 |
| GGT-GCT | GGAAAGGGATTTAGAAGCAGGCAATTTAACAGTGCCTGATATATC | |
| | TTTGATGGGTTGCAGCACAATCTCGTTGGGCT | |
| | ATTGCCTGCTTCTAAAT | 4351 |
| | ATTTAGAAGCAGGCAAT | 4352 |
| Glyphosate Resistance | ATTGTTTCCTTGGTACGAAATGTCCTCCTGTTCGAATTGTCAGCA | 4353 |
| EPSPS 2 | AGGGAGGCCTTCCCGCAGGGAAGGTAAAGCTCTCTGGATCAATT | |
| Nicotiana tabacum | AGCAGCCAGTACTTGACTGCTCTGCTTATGGC | |
| Gly62Ala | GCCATAAGCAGAGCAGTCAAGTACTGGCTGCTAATTGATCCAGA | 4354 |
| GGA-GCA | GAGCTTTACCTTCCCTGCGGGAAGGCCTCCCTTGCTGACAATTC | |
| | GAACAGGAGGACATTTCGTACCAAGGAAACAAT | |
| | CCTTCCCGCAGGGAAGG | 4355 |
| | CCTTCCCTGCGGGAAGG | 4356 |
| Glyphosate Resistance | ATTGTTTCCTTGGCACTGACTGCCCACCTGTTCGTGTCAATGGAA | 4357 |
| EPSPS | TCGGAGGGCTACCTGCTGGCAAGGTCAAGCTGTCTGGCTCCATC | |
| Zea mays | AGCAGTCAGTACTTGAGTGCCTTGCTGATGGC | |
| Gly168Ala | GCCATCAGCAAGGCACTCAAGTACTGACTGCTGATGGAGCCAGA | 4358 |
| GGT-GCT | CAGCTTGACCTTGCCAGCAGGTAGCCCTCCGATTCCATTGACAC | |
| | GAACAGGTGGGCAGTCAGTGCCAAGGAAACAAT | |
| | GCTACCTGCTGGCAAGG | 4359 |
| | CCTTGCCAGCAGGTAGC | 4360 |
| Glyphosate Resistance | ACTGTTTCCTTGGCACTGAATGCCCACCTGTTCGTGTCAAGGGA | 4361 |
| EPSPS | ATTGGAGGACTTCCTGCTGGCAAGGTTAAGCTCTCTGGTTCCAT | |
| Oryza sativa | CAGCAGTCAGTACTTGAGTGCCTTGCTGATGGC | |
| Gly115Ala | GCCATCAGCAAGGCACTGAAGTACTGACTGCTGATGGAACCAGA | 4362 |
| GGT-GCT | GAGCTTAACCTTGCCAGCAGGAAGTCCTCCMTTCCCTTGACAC | |
| | GAACAGGTGGGCATTCAGTGCCAAGGAAACAGT | |
| | ACTTCCTGCTGGCAAGG | 4363 |
| | CCTTGCCAGCAGGAAGT | 4364 |
| Glyphosate Resistance | AGCCTTCTGAGATAGTGTTGCAACCCATTAAAGAGATTTCAGGCA | 4365 |
| EPSPS | CTGTTAAATTGCCTGCCTCTAAATCATTATCTAATAGAATTCTCCT | |
| Petunia x hybrida | TCTTGCTGCCTTATCTGAAGGAACAACTGT | |
| Gly93Ala | ACAGTTGTTCCTTCAGATAAGGCAGCAAGAAGGAGAATTCTATTA | 4366 |
| GGC-GCC | GATAATGATTTAGAGGCAGGCAATTTAACAGTGCCTGAAATCTCT | |
| | TTAATGGGTTGCAACACTATCTCAGAAGGCT | |
| | ATTGCCTGCCTCTAAAT | 4367 |
| | ATTTAGAGGCAGGCAAT | 4368 |
| Glyphosate Resistance | AACCCCATGAGATTGTGCTAGNACCCATCAAAGATATATCTGGTA | 4369 |
| EPSPS | CTGTTAAATTACCCGCTTCGAAATCCCTTTCCAATCGTATTCTCCT | |
| Lycopersicon | TCTTGCTGCCCTTTCTGAGGGAAGGACTGT | |
| esculentum | ACAGTCCTTCCCTCAGAAAGGGCAGCAAGAAGGAGAATACGATT | 4370 |
| Gly97Ala | GGAAAGGGATTTCGAAGCGGGTAATTTAACAGTACCAGATATATC | |
| GGT-GCT | TTTGATGGGTNCTAGCACAATCTCATGGGGTT | |
| | ATTACCCGCTTCGAAAT | 4371 |
| | ATTTCGAAGCGGGTAAT | 4372 |
| Glyphosate Resistance | ATTGTTTCCTTGGCACTGACTGCCCACCTGTTCGKATCAACGGCA | 4373 |
| EPSPS | TTGGAGGGCTACCTGCTGGCAAGGTTAAGCTGTCTGGTTCCATC | |
| Lolium rigidum | AGCAGCCAATACTTGAGTTCCTTGCTGATGGC | |
| Gly107Ala | GCCATCAGCAAGGAACTCAAGTATTGGCTGCTGATGGAACCAGA | 4374 |
| GGT-GCT | CAGCTTAACCTTGCCAGCAGGTAGCCCTCCAATGCCGTTGATCG | |
| | AACAGGTGGGCAGTCAGTGCCAAGGAAACAAT | |
| | GCTACCTGCTGGCAAGG | 4375 |
| | CCTTGCCAGCAGGTAGC | 4376 |

TABLE 29

Genome-Altering Oligos Conferring Imidazolinone
and Sulfonylurea Herbicide Resistance

Phenotype, Gene,

| | |
|---|---|
| Plant & Targeted | SEQ ID |

| | | |
|---|---|---|
| Alteration | Altering Oligos | NO: |

| | | |
|---|---|---|
| Sulfonylurea | AGCGGATTAGCCGATGCGTTGTTAGATAGTGTTCCTCTTGTAGCA | 4377 |
| Resistance | ATCACAGGACAAGTCTCTCGTCGTATGATTGGTACAGATGCGTTT | |
| ALS | CAAGAGACTCCGATTGTTGAGGTAACGCGTT | |
| Arabidopsis thaliana | AACGCGTTACCTCAACAATCGGAGTCTCTTGAAACGCATCTGTAC | 4378 |
| Pro197Ser | CAATCATACGACGAGAGACTTGTCCTGTGATTGCTACAAGAGGAA | |
| CCT-TCT | CACTATCTAACAACGCATCGGCTAATCCGCT | |
| | GACAAGTCTCTCGTCGT | 4379 |
| | ACGACGAGAGACTTGTC | 4380 |
| Sulfonylurea | AGCGGATTAGCCGATGCGTTGTTAGATAGTGTTCCTCTTGTAGCA | 4381 |
| Resistance | ATCACAGGACAAGTCCAGCGTCGTATGATTGGTACAGATGCGTTT | |
| ALS | CAAGAGACTCCGATTGTTGAGGTAACGCGTT | |
| Arabidopsis thaliana | AACGCGTTACCTCAACAATCGGAGTCTCTTGAAACGCATCTGTAC | 4382 |
| Pro197Gln | CAATCATACGACGCTGGACTTGTCCTGTGATTGCTACAAGAGGAA | |
| CCT-CAG | CACTATCTAACAACGCATCGGCTAATCCGCT | |
| | ACAAGTCCAGCGTCGTC | 4383 |
| | TACGACGCTGGACTTGT | 4384 |
| Sulfonylurea | AGCGGATTAGCCGATGCGTTGTTAGATAGTGTTCCTCTTGTAGCA | 4385 |
| Resistance | ATCACAGGACAAGTCCAACGTCGTATGATTGGTACAGATGCGTTT | |
| ALS | CAAGAGACTCCGATTGTTGAGGTAACGCGTT | |
| Arabidopsis thaliana | AACGCGTTACCTCAACAATCGGAGTCTCTTGAAACGCATCTGTAC | 4386 |
| Pro197Gln | CAATCATACGACGTTGGACTTGTCCTGTGATTGCTACAAGAGGAA | |
| CCT-CAA | CACTATCTAACAACGCATCGGCTAATCCGCT | |
| | ACAAGTCCAACGTCGTA | 4387 |
| | TACGACGTTGGACTTGT | 4388 |
| Imidazolinone | GACCTTACCTGTTGGATGTGATTTGTCCGCACCAAGAACATGTGT | 4389 |
| Resistance | TGCCGATGATCCCGAACGGTGGCACTTTCAACGATGTCATAACGG | |
| ALS | AAGGAGATGGCCGGATTAAATACTGAGAGAT | |
| Arabidopsis thaliana | ATCTCTCAGTATTTAATCCGGCCATCTCCTTCCGTTATGACATCGT | 4390 |
| Ser653Asn | TGAAAGTGCCACCGTTCGGGATCATCGGCAACACATGTTCTTGGT | |
| AGT-AAC | GCGGACAAATCACATCCAACAGGTAAGGTC | |
| | GATCCCGAACGGTGGCA | 4391 |
| | TGCCACCGTTCGGGATC | 4392 |
| Imidazolinone | GACCTTACCTGTTGGATGTGATTTGTCCGCACCAAGAACATGTGT | 4393 |
| Resistance | TGCCGATGATCCCGAATGGTGGCACTTTCAACGATGTCATAACGG | |
| ALS | AAGGAGATGGCCGGATTAAATACTGAGAGAT | |
| Arabidopsis thaliana | ATCTCTCAGTATTTAATCCGGCCATCTCCTTCCGTTATGACATCGT | 4394 |
| Ser653Asn | TGAAAGTGCCACCATTCGGGATCATCGGCAACACATGTTCTTGGT | |
| AGT-AAT | GCGGACAAATCACATCCAACAGGTAAGGTC | |
| | GATCCCGAATGGTGGCA | 4395 |
| | TGCCACCATTCGGGATC | 4396 |
| Sulfonylurea | TCCGCGCTCGCCGACGCGCTGCTCGACTCCGTCCCGATGGTCGC | 4397 |
| Resistance | CATCACGGGCCAGGTCTCCCGCCGCATGATCGGCACCGACGCCT | |
| ALS | TCCAGGAGACGCCCATAGTCGAGGTCACCCGCT | |
| Oryza sativa | AGCGGGTGACCTCGACTATGGGCGTCTCCTGGAAGGCGTCGGTG | 4398 |
| Pro171Ser | CCGATCATGCGGCGGGAGACCTGGCCCGTGATGGCGACCATCG | |
| CCC-TCC | GGACGGAGTCGAGCAGCGCGTCGGCGAGCGCGGA | |
| | GCCAGGTCTCCCGCCGC | 4399 |
| | GCGGCGGGAGACCTGGC | 4400 |
| Sulfonylurea | CCGCGCTCGCCGACGCGCTGCTCGACTCCGTCCCGATGGTCGCC | 4401 |
| Resistance | ATCACGGGCCAGGTCCAACGCCGCATGATCGGCACCGACGCCTT | |
| ALS | CCAGGAGACGCCCATAGTCGAGGTCACCCGCTC | |
| Oryza sativa | GAGCGGGTGACCTCGACTATGGGCGTCTCCTGGAAGGCGTCGGT | 4402 |
| Pro171Gln | GCCGATCATGCGGCGTTGGACCTGGCCCGTGATGGCGACCATCG | |
| CCC-CAA | GGACGGAGTCGAGCAGCGCGTCGGCGAGCGCGG | |
| | CCAGGTCCAACGCCGCA | 4403 |
| | TGCGGCGTTGGACCTGG | 4404 |
| Sulfonylurea | CCGCGCTCGCCGACGCGCTGCTCGACTCCGTCCCGATGGTCGCC | 4405 |
| Resistance | ATCACGGGCCAGGTCCAGCGCCGCATGATCGGCACCGACGCCTT | |
| ALS | CCAGGAGACGCCCATAGTCGAGGTCACCCGCTC | |
| Oryza sativa | GAGCGGGTGACCTCGACTATGGGCGTCTCCTGGAAGGCGTCGGT | 4406 |
| Pro171Gln | GCCGATCATGCGGCGCTGGACCTGGCCCGTGATGGCGACCATCG | |
| CCC-CAG | GGACGGAGTCGAGCAGCGCGTCGGCGAGCGCGG | |
| | CCAGGTCCAGCGCCGCA | 4407 |
| | TGCGGCGCTGGACCTGG | 4408 |
| Imidazolinone | GGCCATACTTGTTGGATATCATCGTCCCGCACCAGGAGCATGTGC | 4409 |
| Resistance | TGCCTATGATCCCAAATGGGGGCGCATTCAAGGACATGATCCTGG | |
| ALS | ATGGTGATGGCAGGACTGTGTATTAATCTAT | |
| Oryza sativa | ATAGATTAATACACAGTCCTGCCATCACCATCCAGGATCATGTCCT | 4410 |
| Ile627Asn | TGAATGCGCCCCCATTTGGGATCATAGGCAGCACATGCTCCTGGT | |
| ATT-AAT | GCGGGACGATGATATCCAACAAGTATGGCC | |
| | GATCCCAAATGGGGGCG | 4411 |
| | CGCCCCCATTTGGGATC | 4412 |
| Sulfonylurea | TCCGCGCTCGCCGACGCGCTGCTCGATTCCGTCCCCATGGTCGC | 4413 |
| Resistance | CATCACGGGACAGGTGTCGCGACGCATGATTGGCACCGACGCCT | |
| ALS | TCCAGGAGACGCCCATCGTCGAGGTCACCCGCT | |
| Zea mays | AGCGGGTGACCTCGACGATGGGCGTCTCCTGGAAGGCGTCGGT | 4414 |
| Pro165Ser | GCCAATCATGCGTCGCGACACCTGTCCCGTGATGGCGACCATGG | |
| CCG-TCG | GGACGGAATCGAGCAGCGCGTCGGCGAGCGCGGA | |
| | GACAGGTGTCGCGACGC | 4415 |
| | GCGTCGCGACACCTGTC | 4416 |
| Sulfonylurea | CCGCGCTCGCCGACGCGCTGCTCGATTCCGTCCCCATGGTCGCC | 4417 |
| Resistance | ATCACGGGACAGGTGCAGCGACGCATGATTGGCACCGACGCCTT | |
| ALS | CCAGGAGACGCCCATCGTCGAGGTCACCCGCTC | |
| Zea mays | GAGCGGGTGACCTCGACGATGGGCGTCTCCTGGAAGGCGTCGG | 4418 |
| Pro165Gln | TGCCAATCATGCGTCGCTGCACCTGTCCCGTGATGGCGACCATG | |
| CCG-CAG | GGGACGGAATCGAGCAGCGCGTCGGCGAGCGCGG | |
| | ACAGGTGCAGCGACGCA | 4419 |
| | TGCGTCGCTGCACCTGT | 4420 |
| Imidazolinone | GGCCGTACCTCTTGGATATAATCGTCCCACACCAGGAGCATGTGT | 4421 |
| Resistance | TGCCTATGATCCCTAATGGTGGGGCTTTCAAGGATATGATCCTGG | |
| ALS | ATGGTGATGGCAGGACTGTGTACTGATCTAA | |
| Zea mays | TTAGATCAGTACACAGTCCTGCCATCACCATCCAGGATCATATCCT | 4422 |
| Ser621Asn | TGAAAGCCCCACCATTAGGGATCATAGGCAACACATGCTCCTGGT | |
| AGT-AAT | GTGGGACGATTATATCCAAGAGGTACGGCC | |
| | GATCCCTAATGGTGGGG | 4423 |
| | CCCCACCATTAGGGATC | 4424 |
| Imidazolinone | GGCCGTACCTCTTGGATATAATCGTCCCACACCAGGAGCATGTGT | 4425 |
| Resistance | TGCCTATGATCCCTAACGGTGGGGCTTTCAAGGATATGATCCTGG | |
| ALS | ATGGTGATGGCAGGACTGTGTACTGATCTAA | |
| Zea mays | TTAGATCAGTACACAGTCCTGCCATCACCATCCAGGATCATATCCT | 4426 |
| Ser621Asn | TGAAAGCCCCACCGTTAGGGATCATAGGCAACACATGCTCCTGGT | |
| AGT-AAC | GTGGGACGATTATATCCAAGAGGTACGGCC | |
| | GATCCCTAACGGTGGGG | 4427 |
| | CCCCACCGTTAGGGATC | 4428 |
| Sulfonylurea | TCCGCGCTCGCCGACGCCCTCCTCGACTCCATCCCCATGGTGGC | 4429 |
| Resistance | CATCACGGGGCAGGTCTCGCGCCGCATGATCGGCACGGACGCCT | |
| ALS | TCCAGGAGACGCCCATCGTCGAGGTCACCCGCT | |
| Lolium multiflorum | AGCGGGTGACCTCGACGATGGGCGTCTCCTGGAAGGCGTCCGTG | 4430 |
| Pro167Ser | CCGATCATGCGGCGCGAGACCTGCCCCGTGATGGCCACCATGG | |
| CCG-TCG | GGATGGAGTCGAGGAGGGCGTCGGCGAGCGCGGA | |
| | GGCAGGTCTCGCGCCGC | 4431 |
| | GCGGCGCGAGACCTGCC | 4432 |
| Sulfonylurea | CCGCGCTCGCCGACGCCCTCCTCGACTCCATCCCCATGGTGGCC | 4433 |
| Resistance | ATCACGGGGCAGGTCCAGCGCCGCATGATCGGCACGGACGCCTT | |
| ALS | CCAGGAGACGCCCATCGTCGAGGTCACCCGCTC | |
| Lolium multiflorum | GAGCGGGTGACCTCGACGATGGGCGTCTCCTGGAAGGCGTCCGT | 4434 |
| Pro167Gln | GCCGATCATGCGGCGCTGGACCTGCCCCGTGATGGCCACCATGG | |
| CCG-CAG | GGATGGAGTCGAGGAGGGCGTCGGCGAGCGCGG | |
| | GCAGGTCCAGCGCCGCA | 4435 |
| | TGCGGCGCTGGACCTGC | 4436 |
| Imidazolinone | CTGGGCCATACTTGTTGGATATCATCGTCCCTCACCAGGAGCATG | 4437 |
| Resistance | TGCTGCCTATGATCCCTAACGGTGGTGCTTTCAAGGACATTATCA | |
| ALS | TGGAAGGTGATGGCAGGATTTCGTATTAAAC | |
| Lolium multiflorum | GTTTAATACGAAATCCTGCCATCACCTTCCATGATAATGTCCTTGA | 4438 |
| Ser623Asn | AAGCACCACCGTTAGGGATCATAGGCAGCACATGCTCCTGGTGA | |
| AGC-AAC | GGGACGATGATATCCAACAAGTATGGCCCAG | |
| | GATCCCTAACGGTGGTG | 4439 |
| | CACCACCGTTAGGGATC | 4440 |
| Sulfonylurea | TCCGCGCTCGCCGACGCTCTCCTCGACTCCATCCCCATGGTCGC | 4441 |
| Resistance | CATCACGGGCCAGGTCTCACGCCGCATGATCGGCACGGACGCGT | |
| ALS | TCCAGGAGACGCCCATAGTGGAGGTCACGCGCT | |
| Hordeum vulgare | AGCGCGTGACCTCCACTATGGGCGTCTCCTGGAACGCGTCCGTG | 4442 |
| Pro68Ser | CCGATCATGCGGCGTGAGACCTGGCCCGTGATGGCGACCATGG | |
| CCA-TCA | GGATGGAGTCGAGGAGAGCGTCGGCGAGCGCGGA | |
| | GCCAGGTCTCACGCCGC | 4443 |
| | GCGGCGTGAGACCTGGC | 4444 |
| Sulfonylurea | CCGCGCTCGCCGACGCTCTCCTCGACTCCATCCCCATGGTCGCC | 4445 |
| Resistance | ATCACGGGCCAGGTCCAACGCCGCATGATCGGCACGGACGCGTT | |
| ALS | CCAGGAGACGCCCATAGTGGAGGTCACGCGCTC | |
| Hordeum vulgare | GAGCGCGTGACCTCCACTATGGGCGTCTCCTGGAACGCGTCCGT | 4446 |
| Pro68Gln | GCCGATCATGCGGCGTTGGACCTGGCCCGTGATGGCGACCATGG | |
| CCA-CAA | GGATGGAGTCGAGGAGAGCGTCGGCGAGCGCGG | |
| | CCAGGTCCAACGCCGCA | 4447 |
| | TGCGGCGTTGGACCTGG | 4448 |
| Imidazolinone | CCCAGGGCCGTACCTGCTGGATATCATTGTCCCGCATCAGGAGC | 4449 |
| Resistance | ACGTGCTGCCTATGATCCCAAACGGTGGTGCTTTCAAGGACATGA | |
| ALS | TCATGGAGGGTGATGGCAGGACCTCGTACTGA | |
| Hordeum vulgare | TCAGTACGAGGTCCTGCCATCACCCTCCATGATCATGTCCTTGAA | 4450 |
| Ser524Asn | AGCACCACCGTTTGGGATCATAGGCAGCACGTGCTCCTGATGCG | |
| AGC-AAC | GGACAATGATATCCAGCAGGTACGGCCCTGGG | |
| | GATCCCAAACGGTGGTG | 4451 |
| | CACCACCGTTTGGGATC | 4452 |
| Sulfonylurea | AGTGGTCTCGCTGATGCAATGCTCGATAGTATCCCTCTCGTGGCG | 4453 |
| Resistance | ATCACTGGTCAAGTCTCTCGTCGGATGATCGGTACCGATGCTTTC | |
| ALS | CAGGAAACTCCAATTGTTGAGGTAACAAGGT | |
| Gossypium hirsutum | ACCTTGTTACCTCAACAATTGGAGTTTCCTGGAAAGCATCGGTAC | 4454 |
| Pro186Ser | CGATCATCCGACGAGAGACTTGACCAGTGATCGCCACGAGAGGG | |
| CCT-TCT | ATACTATCGAGCATTGCATCAGCGAGACCACT | |
| | GTCAAGTCTCTCGTCGG | 4455 |
| | CCGACGAGAGACTTGAC | 4456 |
| Sulfonylurea | GTGGTCTCGCTGATGCAATGCTCGATAGTATCCCTCTCGTGGCGA | 4457 |
| Resistance | TCACTGGTCAAGTCCAACGTCGGATGATCGGTACCGATGCTTTCC | |
| ALS | AGGAAACTCCAATTGTTGAGGTAACAAGGTC | |
| Gossypium hirsutum | GACCTTGTTACCTCAACAATTGGAGTTTCCTGGAAAGCATCGGTA | 4458 |
| Pro186Gln | CCGATCATCCGACGTTGGACTTGACCAGTGATCGCCACGAGAGG | |
| CCT-CAA | GATACTATCGAGCATTGCATCAGCGAGACCAC | |
| | TCAAGTCCAACGTCGGA | 4459 |
| | TCCGACGTTGGACTTGA | 4460 |
| Sulfonylurea | GTGGTCTCGCTGATGCAATGCTCGATAGTATCCCTCTCGTGGCGA | 4461 |
| Resistance | TCACTGGTCAAGTCCAGCGTCGGATGATCGGTACCGATGCTTTCC | |
| ALS | AGGAAACTCCAATTGTTGAGGTAACAAGGTC | |
| Gossypium hirsutum | GACCTTGTTACCTCAACAATTGGAGTTTCCTGGAAAGCATCGGTA | 4462 |
| Pro186Gln | CCGATCATCCGACGCTGGACTTGACCAGTGATCGCCACGAGAGG | |
| CCT-CAG | GATACTATCGAGCATTGCATCAGCGAGACCAC | |
| | TCAAGTCCAGCGTCGGA | 4463 |
| | TCCGACGCTGGACTTGA | 4464 |
| Imidazolinone | GACCTTACTTGTTGGATGTGATTGTCCCACATCAAGAACATGTCCT | 4465 |
| Resistance | GCCTATGATCCCCAATGGAGGCGCTTTCAAAGATGTGATCACAGA | |
| ALS | GGGTGATGGAAGAACACAATATTGACCTCA | |
| Gossypium hirsutum | TGAGGTCAATATTGTGTTCTTCCATCACCCTCTGTGATCACATCTT | 4466 |
| Ser642Asn | TGAAAGCGCCTCCATTGGGGATCATAGGCAGGACATGTTCTTGAT | |
| AGT-AAT | GTGGGACAATCACATCCAACAAGTAAGGTC | |
| | GATCCCCAATGGAGGCG | 4467 |
| | CGCCTCCATTGGGGATC | 4468 |
| Sulfonylurea | TCTGGTCTTGCTGATGCACTTCTTGACTCAGTCCCTCTTGTCGCCA | 4469 |
| Resistance | TTACTGGGCAAGTTTCCCGGCGTATGATTGGTACTGATGCTTTTCA | |
| ALS | AGAGACTCCAATTGTTGAGGTAACTCGAT | |
| Amaranthus | ATCGAGTTACCTCAACAATTGGAGTCTCTTGAAAAGCATCAGTACC | 4470 |
| retroflexus | AATCATACGCCGGGAAACTTGCCCAGTAATGGCGACAAGAGGGA | |
| Pro192Ser | CTGAGTCAAGAAGTGCATCAGCAAGACCAGA | |
| CCC-TCC | GGCAAGTTTCCCGGCGT | 4471 |
| | ACGCCGGGAAACTTGCC | 4472 |
| Sulfonylurea | CTGGTCTTGCTGATGCACTTCTTGACTCAGTCCCTCTTGTCGCCAT | 4473 |
| Resistance | TACTGGGCAAGTTCAACGGCGTATGATTGGTACTGATGCTTTTCA | |
| ALS | AGAGACTCCAATTGTTGAGGTAACTCGATC | |
| Amaranthus | GATCGAGTTACCTCAACAATTGGAGTCTCTTGAAAAGCATCAGTAC | 4474 |
| retroflexus | CAATCATACGCCGTTGAACTTGCCCAGTAATGGCGACAAGAGGGA | |
| Pro192Gln | CTGAGTCAAGAAGTGCATCAGCAAGACCAG | |
| CCC-CAA | GCAAGTTCAACGGCGTA | 4475 |
| | TACGCCGTTGAACTTGC | 4476 |
| Sulfonylurea | CTGGTCTTGCTGATGCACTTCTTGACTCAGTCCCTCTTGTCGCCAT | 4477 |
| Resistance | TACTGGGCAAGTTCAGCGGCGTATGATTGGTACTGATGCTTTTCA | |
| ALS | AGAGACTCCAATTGTTGAGGTAACTCGATC | |
| Amaranthus | GATCGAGTTACCTCAACAATTGGAGTCTCTTGAAAAGCATCAGTAC | 4478 |
| retroflexus | CAATCATACGCCGCTGAACTTGCCCAGTAATGGCGACAAGAGGG | |
| Pro192Gln | ACTGAGTCAAGAAGTGCATCAGCAAGACCAG | |
| CCC-CAG | GCAAGTTCAGCGGCGTA | 4479 |
| | TACGCCGCTGAACTTGC | 4480 |
| Imidazolinone | GACCGTATCTGCTGGATGTAATCGTACCACATCAGGAGCATGTGC | 4481 |
| Resistance | TGCCTATGATCCCTAACGGTGCCGCCTTCAAGGACACCATAACAG | |
| ALS | AGGGTGATGGAAGAAGGGCTTATTAGTTGGT | |
| Amaranthus | ACCAACTAATAAGCCCTTCTTCCATCACCCTCTGTTATGGTGTCCT | 4482 |
| retroflexus | TGAAGGCGGCACCGTTAGGGATCATAGGCAGCACATGCTCCTGA | |
| Ser652Asn | TGTGGTACGATTACATCCAGCAGATACGGTC | |
| AGC-AAC | GATCCCTAACGGTGCCG | 4483 |
| | CGGCACCGTTAGGGATC | 4484 |
| Sulfonylurea | AGCGGCCTCGCTGACGCGCTACTGGATAGCGTCCCCATTGTTGC | 4485 |
| Resistance | TATAACAGGTCAAGTGTCACGTAGGATGATAGGTACTGATGCTTTT | |
| ALS 1 | CAGGAAACTCCTATTGTTGAGGTAACTAGAT | |
| Nicotiana tabacum | ATCTAGTTACCTCAACAATAGGAGTTTCCTGAAAAGCATCAGTACC | 4486 |
| Pro194Ser | TATCATCCTACGTGACACTTGACCTGTTATAGCAACAATGGGGAC | |
| CCA-TCA | GCTATCCAGTAGCGCGTCAGCGAGGCCGCT | |
| | GTCAAGTGTCACGTAGG | 4487 |
| | CCTACGTGACACTTGAC | 4488 |
| Sulfonylurea | GCGGCCTCGCTGACGCGCTACTGGATAGCGTCCCCATTGTTGCT | 4489 |
| Resistance | ATAACAGGTCAAGTGCAACGTAGGATGATAGGTACTGATGCTTTT | |
| ALS 1 | CAGGAAACTCCTATTGTTGAGGTAACTAGATC | |
| Nicotiana tabacum | GATCTAGTTACCTCAACAATAGGAGTTTCCTGAAAAGCATCAGTAC | 4490 |
| Pro194Gln | CTATCATCCTACGTTGCACTTGACCTGTTATAGCAACAATGGGGA | |
| CCA-CAA | CGCTATCCAGTAGCGCGTCAGCGAGGCCGC | |
| | TCAAGTGCAACGTAGGA | 4491 |
| | TCCTACGTTGCACTTGA | 4492 |
| Imidazolinone | GGCCATACTTGTTGGATGTGATTGTACCTCATCAGGAACATGTTTT | 4493 |
| Resistance | ACCTATGATTCCCAATGGCGGAGCTTTCAAAGATGTGATCACAGA | |
| ALS 1 | GGGTGACGGGAGAAGTTCCTATTGAGTTTG | |
| Nicotiana tabacum | CAAACTCAATAGGAACTTCTCCCGTCACCCTCTGTGATCACATCTT | 4494 |
| Ser650Asn | TGAAAGCTCCGCCATTGGGAATCATAGGTAAAACATGTTCCTGAT | |
| AGT-AAT | GAGGTACAATCACATCCAACAAGTATGGCC | |
| | GATTCCCAATGGCGGAG | 4495 |
| | CTCCGCCATTGGGAATC | 4496 |
| Sulfonylurea | AGTGGCCTCGCGGACGCCCTACTGGATAGCGTCCCCATTGTTGC | 4497 |
| Resistance | TATAACCGGTCAAGTGTCACGTAGGATGATCGGTACTGATGCTTT | |
| ALS 2 | TCAGGAAACTCCGATTGTTGAGGTAACTAGAT | |
| Nicotiana tabacum | ATCTAGTTACCTCAACAATCGGAGTTTCCTGAAAAGCATCAGTACC | 4498 |
| Pro191Ser | GATCATCCTACGTGACACTTGACCGGTTATAGCAACAATGGGGAC | |
| CCA-TCA | GCTATCCAGTAGGGCGTCCGCGAGGCCACT | |
| | GTCAAGTGTCACGTAGG | 4499 |
| | CCTACGTGACACTTGAC | 4500 |
| Sulfonylurea | GTGGCCTCGCGGACGCCCTACTGGATAGCGTCCCCATTGTTGCT | 4501 |
| Resistance | ATAACCGGTCAAGTGCAACGTAGGATGATCGGTACTGATGCTTTT | |
| ALS 2 | CAGGAAACTCCGATTGTTGAGGTAACTAGATC | |
| Nicotiana tabacum | GATCTAGTTACCTCAACAATCGGAGTTTCCTGAAAAGCATCAGTAC | 4502 |
| Pro191Gln | CGATCATCCTACGTTGCACTTGACCGGTTATAGCAACAATGGGGA | |
| CCA-CAA | CGCTATCCAGTAGGGCGTCCGCGAGGCCAC | |
| | TCAAGTGCAACGTAGGA | 4503 |
| | TCCTACGTTGCACTTGA | 4504 |
| Imidazolinone | GGCCATACTTGTTGGATGTGATTGTACCTCATCAGGAACATGTTCT | 4505 |
| Resistance | ACCTATGATTCCCAATGGCGGGGCTTTCAAAGATGTGATCACAGA | |
| ALS 2 | GGGTGACGGGAGAAGTTCCTATTGACTTTG | |
| Nicotiana tabacum | CAAAGTCAATAGGAACTTCTCCCGTCACCCTCTGTGATCACATCTT | 4506 |
| Ser647Asn | TGAAAGCCCCGCCATTGGGAATCATAGGTAGAACATGTTCCTGAT | |
| AGT-AAT | GAGGTACAATCACATCCAACAAGTATGGCC | |
| | GATTCCCAATGGCGGGG | 4507 |
| | CCCCGCCATTGGGAATC | 4508 |
| Sulfonylurea | AGTGGTCTTGCTGATGCTTTATTAGACAGTGTTCCAATGGTTGCTA | 4509 |
| Resistance | TTACTGGTCAAGTTTCCAGGAGAATGATTGGAACAGATGCGTTTC | |
| ALS | AAGAAACCCCTATTGTTGAGGTAACACGTT | |
| Xanthium spp. | AACGTGTTACCTCAACAATAGGGGTTTCTTGAAACGCATCTGTTCC | 4510 |
| Pro175Ser | AATCATTCTCCTGGAAACTTGACCAGTAATAGCAACCATTGGAACA | |
| CCC-TCC | CTGTCTAATAAAGCATCAGCAAGACCACT | |
| | GTCAAGTTTCCAGGAGA | 4511 |
| | TCTCCTGGAAACTTGAC | 4512 |
| Sulfonylurea | GTGGTCTTGCTGATGCTTTATTAGACAGTGTTCCAATGGTTGCTAT | 4513 |
| Resistance | TACTGGTCAAGTTCAAAGGAGAATGATTGGAACAGATGCGTTTCA | |
| ALS | AGAAACCCCTATTGTTGAGGTAACACGTTC | |
| Xanthium spp. | GAACGTGTTACCTCAACAATAGGGGTTTCTTGAAACGCATCTGTTC | 4514 |
| Pro175Gln | CAATCATTCTCCTTTGAACTTGACCAGTAATAGCAACCATTGGAAC | |
| CCC-CAA | ACTGTCTAATAAAGCATCAGCAAGACCAC | |
| | TCAAGTTCAAAGGAGAA | 4515 |
| | TTCTCCTTTGAACTTGA | 4516 |
| Sulfonylurea | GTGGTCTTGCTGATGCTTTATTAGACAGTGTTCCAATGGTTGCTAT | 4517 |
| Resistance | TACTGGTCAAGTTCAGAGGAGAATGATTGGAACAGATGCGTTTCA | |
| ALS | AGAAACCCCTATTGTTGAGGTAACACGTTC | |
| Xanthium spp. | GAACGTGTTACCTCAACAATAGGGGTTTCTTGAAACGCATCTGTTC | 4518 |
| Pro175Gln | CAATCATTCTCCTCTGAACTTGACCAGTAATAGCAACCATTGGAAC | |
| CCC-CAG | ACTGTCTAATAAAGCATCAGCAAGACCAC | |
| | TCAAGTTCAGAGGAGAA | 4519 |
| | TTCTCCTCTGAACTTGA | 4520 |
| Imidazolinone | GGGCCTTACTTGTTGGATGTGATCGTGCCCCATCAAGAACATGTG | 4521 |
| Resistance | TTGCCCATGATCCCGAATGGTGGAGGTTTCATGGATGTGATCACC | |
| ALS | GAAGGCGACGGCAGAATGAAATATTGAGCTT | |
| Xanthium spp. | AAGCTCAATATTTCATTCTGCCGTCGCCTTCGGTGATCACATCCAT | 4522 |
| Ala631Asn | GAAACCTCCACCATTCGGGATCATGGGCAACACATGTTCTTGATG | |
| GCT-AAT | GGGCACGATCACATCCAACAAGTAAGGCCC | |
| | TGATCCCGAATGGTGGA | 4523 |
| | TCCACCATTCGGGATCA | 4524 |
| Sulfonylurea | TCCGGGTTTGCTGATGCTTTGCTCGATTCCGTTCCACTGGTGGCG | 4525 |
| Resistance | ATCACGGGGCAGGTGTCGCGGCGAATGATTGGGACGGATGCTTT | |
| ALS | TCAGGAGACTCCTATTGTTGAGGTAACACGGT | |
| Bassia scoparia | ACCGTGTTACCTCAACAATAGGAGTCTCCTGAATAAGCATCCGTCC | 4526 |
| Pro189Ser | CAATCATTCGCCGCGACACCTGCCCCGTGATCGCCACCAGTGGA | |
| CCG-TCG | ACGGAATCGAGCAAAGCATCAGCAAACCCGGA | |
| | GGCAGGTGTCGCGGCGA | 4527 |
| | TCGCCGCGACACCTGCC | 4528 |
| Sulfonylurea | CCGGGTTTGCTGATGCTTTGCTCGATTCCGTTCCACTGGTGGCGA | 4529 |
| Resistance | TCACGGGGCAGGTGCAGCGGCGAATGATTGGGACGGATGCTTTT | |
| ALS | CAGGAGACTCCTATTGTTGAGGTAACACGGTC | |
| Bassia scoparia | GACCGTGTTACCTCAACAATAGGAGTCTCCTGAAAAGCATCCGTC | 4530 |
| Pro189Gln | CCAATCATTCGCCGCTGCACCTGCCCCGTGATCGCCACCAGTGG | |
| CCG-CAG | AACGGAATCGAGCAAAGCATCAGCAAACCCGG | |
| | GCAGGTGCAGCGGCGAA | 4531 |
| | TTCGCCGCTGCACCTGC | 4532 |
| Imidazolinone | GACCTTACCTGCTTGATGTGATTGTACCTCATCAGGAGCATGTGC | 4533 |
| Resistance | TGCCTATGATTCCTAATGGTGCAGCCTTCAAGGATATCATTAACGA | |
| ALS | AGGTGATGGAAGAACAAGTTATTGATGTTC | |
| Bassia scoparia | GAACATCAATAACTTGTTCTTCCATCACCTTCGTTAATGATATCCTT | 4534 |
| Ser649Asn | GAAGGCTGCACCATTAGGAATCATAGGCAGCACATGCTCCTGATG | |
| AGT-AAT | AGGTACAATCACATCAAGCAGGTAAGGTC | |
| | GATTCCTAATGGTGCAG | 4535 |
| | CTGCACCATTAGGAATC | 4536 |
| Sulfonylurea | AGCGGGTTAGCAGACGCGATGCTTGACAGTGTTCCTCTTGTCGCC | 4537 |
| Resistance | ATTACAGGACAGGTCTCTCGCCGGATGATCGGTACTGACGCCTTC | |
| ALS 1 | CAAGAGACACCAATCGTTGAGGTAACGAGGT | |
| Brassica napus | ACCTCGTTACCTCAACGATTGGTGTCTCTTGGAAGGCGTCAGTAC | 4538 |
| Pro182Ser | CGATCATCCGGCGAGAGACCTGTCCTGTAATGGCGACAAGAGGA | |
| CCT-TCT | ACACTGTCAAGCATCGCGTCTGCTAACCCGCT | |
| | GACAGGTCTCTCGCCGG | 4539 |
| | CCGGCGAGAGACCTGTC | 4540 |
| Sulfonylurea | GCGGGTTAGCAGACGCGATGCTTGACAGTGTTCCTCTTGTCGCCA | 4541 |
| Resistance | TTACAGGACAGGTCCAACGCCGGATGATCGGTACTGACGCCTTC | |
| ALS 1 | CAAGAGACACCAATCGTTGAGGTAACGAGGTC | |
| Brassica napus | GACCTCGTTACCTCAACGATTGGTGTCTCTTGGAAGGCGTCAGTA | 4542 |
| Pro182Gln | CCGATCATCCGGCGTTGGACCTGTCCTGTAATGGCGACAAGAGG | |
| CCT-CAA | AACACTGTCAAGCATCGCGTCTGCTAACCCGC | |
| | ACAGGTCCAACGCCGGA | 4543 |
| | TCCGGCGTTGGACCTGT | 4544 |
| Sulfonylurea | GCGGGTTAGCAGACGCGATGCTTGACAGTGTTCCTCTTGTCGCCA | 4545 |
| Resistance | TTACAGGACAGGTCCAGCGCCGGATGATCGGTACTGACGCCTTC | |
| ALS 1 | CAAGAGACACCAATCGTTGAGGTAACGAGGTC | |
| Brassica napus | GACCTCGTTACCTCAACGATTGGTGTCTCTTGGAAGGCGTCAGTA | 4546 |
| Pro182Gln | CCGATCATCCGGCGCTGGACCTGTCCTGTAATGGCGACAAGAGG | |
| CCT-CAG | AACACTGTCAAGCATCGCGTCTGCTAACCCGC | |
| | ACAGGTCCAGCGCCGGA | 4547 |
| | TCCGGCGCTGGACCTGT | 4548 |
| Imidazolinone | GACCATACCTGTTGGATGTGATATGTCCGCACCAAGAACATGTGT | 4549 |
| Resistance | TACCGATGATCCCAAATGGTGGCACTTTCAAAGATGTAATAACAG | |
| ALS 1 | AAGGGGATGGTCGCACTAAGTACTGAGAGAT | |
| Brassica napus | ATCTCTCAGTACTTAGTGCGACCATCCCCTTCTGTTATTACATCTTT | 4550 |
| Ser638Asn | GAAAGTGCCACCATTTGGGATCATCGGTAACACATGTTCTTGGTG | |
| AGT-AAT | CGGACATATCACATCCAACAGGTATGGTC | |
| | GATCCCAAATGGTGGCA | 4551 |
| | TGCCACCATTTGGGATC | 4552 |
| Sulfonylurea | CAGCGGGTTAGCAGACGCGATGCTTGACAGTGTTCCTCTTGTCGC | 4553 |
| Resistance | CATTACAGGACAGGTTCCTCGCCGGATGATCGGTACTGACGCCTT | |
| ALS 2 | CCAAGAGACACCAATCGTTGAGGTAACGAGG | |
| Brassica napus | CCTCGTTACCTCAACGATTGGTGTCTCTTGGAAGGCGTCAGTACC | 4554 |
| Pro126Ser | GATCATCCGGCGAGGAACCTGTCCTGTAATGGCGACAAGAGGAA | |
| CCC-TCC | CACTGTCAAGCATCGCGTCTGCTAACCCGCTG | |
| | GGACAGGTTCCTCGCCG | 4555 |
| | CGGCGAGGAACCTGTCC | 4556 |
| Sulfonylurea | AGCGGGTTAGCAGACGCGATGCTTGACAGTGTTCCTCTTGTCGCC | 4557 |
| Resistance | ATTACAGGACAGGTCACTCGCCGGATGATCGGTACTGACGCCTTC | |
| ALS 2 | CAAGAGACACCAATCGTTGAGGTAACGAGGT | |
| Brassica napus | ACCTCGTTACCTCAACGATTGGTGTCTCTTGGAAGGCGTCAGTAC | 4558 |
| Pro126Gln | CGATCATCCGGCGAGTGACCTGTCCTGTAATGGCGACAAGAGGA | |
| CCC-CAG | ACACTGTCAAGCATCGCGTCTGCTAACCCGCT | |
| | GACAGGTCACTCGCCGG | 4559 |
| | CCGGCGAGTGACCTGTC | 4560 |
| Imidazolinone | GACCATACCTGTTGGATGTGATATGTCCGCACCAAGAACATGTGT | 4561 |
| Resistance | TACCGATGATCCCAAATGGTGGCACTTTCAAAGATGTAATAACAG | |
| ALS 2 | AAGGGGATGGTCGCACTAAGTACTGAGAGAT | |
| Brassica napus | ATCTCTCAGTACTTAGTGCGACCATCCCCTTCTGTTATTACATCTTT | 4562 |
| Ser582Asn | GAAAGTGCCACCATTTGGGATCATCGGTAACACATGTTCTTGGTG | |
| AGT-AAT | CGGACATATCACATCCAACAGGTATGGTC | |
| | GATCCCAAATGGTGGCA | 4563 |
| | TGCCACCATTTGGGATC | 4564 |
| Sulfonylurea | AGCGGGTTAGCCGACGCGATGCTTGACAGTGTTCCTCTCGTCGC | 4565 |
| Resistance | CATCACAGGACAGGTCTCTCGCCGGATGATCGGTACTGACGCGT | |
| ALS 3 | TCCAAGAGACGCCAATCGTTGAGGTAACGAGGT | |
| Brassica napus | ACCTCGTTACCTCAACGATTGGCGTCTCTTGGAACGCGTCAGTAC | 4566 |
| Pro179Ser | CGATCATCCGGCGAGAGACCTGTCCTGTGATGGCGACGAGAGGA | |
| CCT-TCT | ACACTGTCAAGCATCGCGTCGGCTAACCCGCT | |
| | GACAGGTCTCTCGCCGG | 4567 |
| | CCGGCGAGAGACCTGTC | 4568 |
| Sulfonylurea | GCGGGTTAGCCGACGCGATGCTTGACAGTGTTCCTCTCGTCGCC | 4569 |
| Resistance | ATCACAGGACAGGTCCAACGCCGGATGATCGGTACTGACGCGTT | |
| ALS 3 | CCAAGAGACGCCAATCGTTGAGGTAACGAGGTC | |
| Brassica napus | GACCTCGTTACCTCAACGATTGGCGTCTCTTGGAACGCGTCAGTA | 4570 |
| Pro179Gln | CCGATCATCCGGCGTTGGACCTGTCCTGTGATGGCGACGAGAGG | |
| CCT-CAA | AACACTGTCAAGCATCGCGTCGGCTAACCCGC | |
| | ACAGGTCCAACGCCGGA | 4571 |
| | TCCGGCGTTGGACCTGT | 4572 |
| Sulfonylurea | GCGGGTTAGCCGACGCGATGCTTGACAGTGTTCCTCTCGTCGCC | 4573 |
| Resistance | ATCACAGGACAGGTCCAGCGCCGGATGATCGGTACTGACGCGTT | |
| ALS 3 | CCAAGAGACGCCAATCGTTGAGGTAACGAGGTC | |
| Brassica napus | GACCTCGTTACCTCAACGATTGGCGTCTCTTGGAACGCGTCAGTA | 4574 |
| Pro179Gln | CCGATCATCCGGCGCTGGACCTGTCCTGTGATGGCGACGAGAGG | |
| CCT-CAG | AACACTGTCAAGCATCGCGTCGGCTAACCCGC | |
| | ACAGGTCCAGCGCCGGA | 4575 |
| | TCCGGCGCTGGACCTGT | 4576 |
| Imidazolinone | GACCGTACCTGTTGGATGTCATCTGTCCGCACCAAGAACATGTGT | 4577 |
| Resistance | TACCGATGATCCCAAATGGTGGCACTTTCAAAGATGTAATAACCG | |
| ALS 3 | AAGGGGATGGTCGCACTAAGTACTGAGAGAT | |
| Brassica napus | ATCTCTCAGTACTTAGTGCGACCATCCCCTTCGGTTATTACATCTT | 4578 |
| Ser635Asn | TGAAAGTGCCACCATTTGGGATCATCGGTAACACATGTTCTTGGT | |
| AGT-AAT | GCGGACAGATGACATCCAACAGGTACGGTC | |
| | GATCCCAAATGGTGGCA | 4579 |
| | TGCCACCATTTGGGATC | 4580 |
| Sulfonylurea | TCCGCGCTCGCCGACGCGCTGCTCGACTCCGTCCCGATGGTCGC | 4581 |
| Resistance | CATCACGGGCCAGGTCTCCCGCCGCATGATCGGCACCGACGCCT | |
| ALS | TCCAGGAGACGCCCATAGTCGAGGTCACCCGCT | |
| Oryza sativa | AGCGGGTGACCTCGACTATGGGCGTCTCCTGGAAGGCGTCGGTG | 4582 |
| Pro171Ser | CCGATCATGCGGCGGGAGACCTGGCCCGTGATGGCGACCATCG | |
| CCC-TCC | GGACGGAGTCGAGCAGCGCGTCGGCGAGCGCGGA | |
| | GCCAGGTCTCCCGCCGC | 4583 |
| | GCGGCGGGAGACCTGGC | 4584 |
| Sulfonylurea | CCGCGCTCGCCGACGCGCTGCTCGACTCCGTCCCGATGGTCGCC | 4585 |
| Resistance | ATCACGGGCCAGGTCCAACGCCGCATGATCGGCACCGACGCCTT | |
| ALS | CCAGGAGACGCCCATAGTCGAGGTCACCCGCTC | |
| Oryza sativa | GAGCGGGTGACCTCGACTATGGGCGTCTCCTGGAAGGCGTCGGT | 4586 |
| Pro171Gln | GCCGATCATGCGGCGTTGGACCTGGCCCGTGATGGCGACCATCG | |
| CCC-CAA | GGACGGAGTCGAGCAGCGCGTCGGCGAGCGCGG | |
| | CCAGGTCCAACGCCGCA | 4587 |
| | TGCGGCGTTGGACCTGG | 4588 |
| Sulfonylurea | CCGCGCTCGCCGACGCGCTGCTCGACTCCGTCCCGATGGTCGCC | 4589 |
| Resistance | ATCACGGGCCAGGTCCAGCGCCGCATGATCGGCACCGACGCCTT | |
| ALS | CCAGGAGACGCCCATAGTCGAGGTCACCCGCTC | |
| Oryza sativa | GAGCGGGTGACCTCGACTATGGGCGTCTCCTGGAAGGCGTCGGT | 4590 |
| Pro171Gln | GCCGATCATGCGGCGCTGGACCTGGCCCGTGATGGCGACCATCG | |
| CCC-CAG | GGACGGAGTCGAGCAGCGCGTCGGCGAGCGCGG | |
| | CCAGGTCCAGCGCCGCA | 4591 |
| | TGCGGCGCTGGACCTGG | 4592 |
| Imidazolinone | GGCCATACTTGTTGGATATCATCGTCCCGCACCAGGAGCATGTGC | 4593 |
| Resistance | TGCCTATGATCCCAAATGGGGGCGCATTCAAGGACATGATCCTGG | |
| ALS | ATGGTGATGGCAGGACTGTGTATTAATCTAT | |
| Oryza sativa | ATAGATTAATACACAGTCCTGCCATCACCATCCAGGATCATGTCCT | 4594 |
| Ser627Asn | TGAATGCGCCCCCATTTGGGATCATAGGCAGCACATGCTCCTGGT | |
| AGT-AAT | GCGGGACGATGATATCCAACAAGTATGGCC | |
| | GATCCCAAATGGGGGCG | 4595 |
| | CGCCCCCATTTGGGATC | 4596 |
| Sulfonylurea | TCTGCGCTCGCAGACGCGTTGCTCGACTCCGTCCCCATGGTCGC | 4597 |
| Resistance | CATCACGGGACAGGTGTCGCGACGCATGATTGGCACCGACGCCT | |
| ALS | TTCAGGAGACGCCCATCGTCGAGGTCACCCGCT | |
| Zea mays | AGCGGGTGACCTCGACGATGGGCGTCTCCTGAAAGGCGTCGGTG | 4598 |
| Pro165Ser | CCAATCATGCGTCGCGACACCTGTCCCGTGATGGCGACCATGGG | |
| CCG-TCG | GACGGAGTCGAGCAACGCGTCTGCGAGCGCAGA | |
| | GACAGGTGTCGCGACGC | 4599 |
| | GCGTCGCGACACCTGTC | 4600 |
| Sulfonylurea | CTGCGCTCGCAGACGCGTTGCTCGACTCCGTCCCCATGGTCGCC | 4601 |
| Resistance | ATCACGGGACAGGTGCAGCGACGCATGATTGGCACCGACGCCTT | |
| ALS | TCAGGAGACGCCCATCGTCGAGGTCACCCGCTC | |
| Zea mays | GAGCGGGTGACCTCGACGATGGGCGTCTCCTGAAAGGCGTCGGT | 4602 |
| Pro165Gln | GCCAATCATGCGTCGCTGCACCTGTCCCGTGATGGCGACCATGG | |
| CCG-CAG | GGACGGAGTCGAGCAACGCGTCTGCGAGCGCAG | |
| | ACAGGTGCAGCGACGCA | 4603 |
| | TGCGTCGCTGCACCTGT | 4604 |
| Imidazolinone | GGCCGTACCTCTTGGATATAATCGTCCCGCACCAGGAGCATGTGT | 4605 |
| Resistance | TGCCTATGATCCCTAATGGTGGGGCTTTCAAGGATATGATCCTGG | |
| ALS | ATGGTGATGGCAGGACTGTGTATTGATCCGT | |
| Zea mays | ACGGATCAATACACAGTCCTGCCATCACCATCCAGGATCATATCC | 4606 |
| Ser621Asn | TTGAAAGCCCCACCATTAGGGATCATAGGCAACACATGCTCCTGG | |
| AGT-AAT | TGCGGGACGATTATATCCAAGAGGTACGGCC | |
| | GATCCCTAATGGTGGGG | 4607 |
| | CCCCACCATTAGGGATC | 4608 |
| Sulfonylurea | AGTGGTCTCGCTGATGCAATGCTCGATAGTATCCCTCTCGTGGCG | 4609 |
| Resistance | ATCACTGGTCAAGTCTCTCGTCGGATGATCGGTACCGATGCTTTC | |
| ALS | CAGGAAACTCCAATTGTTGAGGTAACAAGGT | |
| Gossypium hirsutum | ACCTTGTTACCTCAACAATTGGAGTTTCCTGGAAAGCATCGGTAC | 4610 |
| Pro186Ser | CGATCATCCGACGAGAGACTTGACCAGTGATCGCCACGAGAGGG | |
| CCT-TCT | ATACTATCGAGCATTGCATCAGCGAGACCACT | |
| | GTCAAGTCTCTCGTCGG | 4611 |
| | CCGACGAGAGACTTGAC | 4612 |
| Sulfonylurea | GTGGTCTCGCTGATGCAATGCTCGATAGTATCCCTCTCGTGGCGA | 4613 |
| Resistance | TCACTGGTCAAGTCCAACGTCGGATGATCGGTACCGATGCTTTCC | |
| ALS | AGGAAACTCCAATTGTTGAGGTAACAAGGTC | |
| Gossypium hirsutum | GACCTTGTTACCTCAACAATTGGAGTTTCCTGGAAAGCATCGGTA | 4614 |
| Pro186Gln | CCGATCATCCGACGTTGGACTTGACCAGTGATCGCCACGAGAGG | |
| CCT-CAA | GATACTATCGAGCATTGCATCAGCGAGACCAC | |
| | TCAAGTCCAACGTCGGA | 4615 |
| | TCCGACGTTGGACTTGA | 4616 |
| Sulfonylurea | GTGGTCTCGCTGATGCAATGCTCGATAGTATCCCTCTCGTGGCGA | 4617 |
| Resistance | TCACTGGTCAAGTCCAGCGTCGGATGATCGGTACCGATGCTTTCC | |
| ALS | AGGAAACTCCAATTGTTGAGGTAACAAGGTC | |
| Gossypium hirsutum | GACCTTGTTACCTCAACAATTGGAGTTTCCTGGAAAGCATCGGTA | 4618 |
| Pro186Gln | CCGATCATCCGACGCTGGACTTGACCAGTGATCGCCACGAGAGG | |
| CCT-CAG | GATACTATCGAGCATTGCATCAGCGAGACCAC | |
| | TCAAGTCCAGCGTCGGA | 4619 |
| | TCCGACGCTGGACTTGA | 4620 |
| Imidazolinone | GACCTTACTTGTTGGATGTGATTGTCCCACATCAAGAACATGTCCT | 4621 |
| Resistance | GCCTATGATCCCCAATGGAGGGGCTTTCAAAGATGTGATCACAGA | |
| ALS | GGGTGATGGAAGAACACAATATTGACCTCA | |
| Gossypium hirsutum | TGAGGTCAATATTGTGTTCTTCCATCACCCTCTGTGATCACATCTT | 4622 |
| Ser642Asn | TGAAAGCCCCTCCATTGGGGATCATAGGCAGGACATGTTCTTGAT | |
| AGT-AAT | GTGGGACAATCACATCCAACAAGTAAGGTC | |
| | GATCCCCAATGGAGGGG | 4623 |
| | CCCCTCCATTGGGGATC | 4624 |
| Sulfonylurea | TCTGGTCTTGCTGATGCACTTCTTGACTCAGTCCCTCTTGTCGCCA | 4625 |
| Resistance | TTACTGGGCAAGTTTCCCGGCGTATGATTGGTACTGATGCTTTTCA | |
| ALS | AGAGACTCCAATTGTTGAGGTAACTCGAT | |
| Amaranthus powellii | ATCGAGTTACCTCAACAATTGGAGTCTCTTGAAAAGCATCAGTACC | 4626 |
| Pro192Ser | AATCATACGCCGGGAAACTTGCCCAGTAATGGCGACAAGAGGGA | |
| CCC-TCC | CTGAGTCAAGAAGTGCATCAGCAAGACCAGA | |
| | GGCAAGTTTCCCGGCGT | 4627 |
| | ACGCCGGGAAACTTGCC | 4628 |
| Sulfonylurea | CTGGTCTTGCTGATGCACTTCTTGACTCAGTCCCTCTTGTCGCCAT | 4629 |
| Resistance | TACTGGGCAAGTTCAACGGCGTATGATTGGTACTGATGCTTTTCA | |
| ALS | AGAGACTCCAATTGTTGAGGTAACTCGATC | |
| Amaranthus powellii | GATCGAGTTACCTCAACAATTGGAGTCTCTTGAAAAGCATCAGTAC | 4630 |
| Pro192Gln | CAATCATACGCCGTTGAACTTGCCCAGTAATGGCGACAAGAGGGA | |
| CCC-CAA | CTGAGTCAAGAAGTGCATCAGCAAGACCAG | |
| | GCAAGTTCAACGGCGTA | 4631 |
| | TACGCCGTTGAACTTGC | 4632 |
| Sulfonylurea | CTGGTCTTGCTGATGCACTTCTTGACTCAGTCCCTCTTGTCGCCAT | 4633 |
| Resistance | TACTGGGCAAGTTCAGCGGCGTATGATTGGTACTGATGCTTTTCA | |
| ALS | AGAGACTCCAATTGTTGAGGTAACTCGATC | |
| Amaranthus powellii | GATCGAGTTACCTCAACAATTGGAGTCTCTTGAAAAGCATCAGTAC | 4634 |
| Pro192Gln | CAATCATACGCCGCTGAACTTGCCCAGTAATGGCGACAAGAGGG | |
| CCC-CAG | ACTGAGTCAAGAAGTGCATCAGCAAGACCAG | |
| | GCAAGTTCAGCGGCGTA | 4635 |
| | TACGCCGCTGAACTTGC | 4636 |
| Imidazolinone | GACCGTATCTGCTGGATGTAATCGTACCACATCAGGAGCATGTGC | 4637 |
| Resistance | TGCCTATGATCCCTAACGGTGCCGCCTTCAAGGACACCATAACAG | |
| ALS | AGGGTGATGGAAGAAGGGCTTATTAGTTGGT | |
| Amaranthus powellii | ACCAACTAATAAGCCCTTCTTCCATCACCCTCTGTTATGGTGTCCT | 4638 |
| Ser652Asn | TGAAGGCGGCACCGTTAGGGATCATAGGCAGCACATGCTCCTGA | |
| AGC-AAC | TGTGGTACGATTACATCCAGCAGATACGGTC | |
| | GATCCCTAACGGTGCCG | 4639 |
| | CGGCACCGTTAGGGATC | 4640 |

TABLE 30

Genome-Altering Oligos Conferring Porphyric
Herbicide Resistance

| | |
|---|---|
| Phenotype, Gene, | SEQ |

| | | |
|---|---|---|
| Plant & Targeted | | ID |
| Alteration | Altering Oligos | NO: |

| | | |
|---|---|---|
| Porphyric Herbicide | TCTTGCGCCCTCTTTCTGAATCTGCTGCAAATGCACTCTCAAAACT | 4641 |
| Resistant | ATATTACCCACCAATGGCAGCAGTATCTATCTCGTACCCGAAAGA | |
| PPO | AGCAATCCGAACAGAATGTTTGATAGATGG | |
| Arabidopsis thaliana | CCATCTATCAAACATTCTGTTCGGATTGCTTCTTTCGGGTACGAGA | 4642 |
| Val365Met | TAGATACTGCTGCCATTGGTGGGTAATATAGTTTTGAGAGTGCATT | |
| GTT-ATG | TGCAGCAGATTCAGAAAGAGGGCGCAAGA | |
| | CCCACCAATGGCAGCAG | 4643 |
| | CTGCTGCCATTGGTGGG | 4644 |
| Porphyric Herbicide | TATTACGTCCTCTTTCGGTTGCCGCAGCAGATGCACTTTCAAATTT | 4645 |
| Resistant | CTACTATCCCCCAATGGGAGCAGTCACAATTTCATATCCTCAAGAA | |
| PPO | GCTATTCGTGATGAGCGTCTGGTTGATGG | |
| Nicotiana tabacum | CCATCAACCAGACGCTCATCACGAATAGCTTCTTGAGGATATGAA | 4646 |
| Val376Met | ATTGTGACTGCTCCCATTGGGGGATAGTAGAAATTTGAAAGTGCA | |
| GTT-ATG | TCTGCTGCGGCAACCGAAAGAGGACGTAATA | |
| | TCCCCCAATGGGAGCAG | 4647 |
| | CTGCTCCCATTGGGGGA | 4648 |
| Porphyric Herbicide | TGTTGCGTCCGCTTTCGTTGGGTGCAGCAGATGCATTGTCAAAAT | 4649 |
| Resistant | TTTATTATCCTCCGATGGCAGCTGTATCAATTTCATATCCAAAAGA | |
| PPO | CGCAATTCGTGCTGACCGGCTGATTGATGG | |
| Cichorium intybus | CCATCAATCAGCCGGTCAGCACGAATTGCGTCTTTTGGATATGAA | 4650 |
| Val383Met | ATTGATACAGCTGCCATCGGAGGATAATAAAATTTTGACAATGCAT | |
| GTT-ATG | CTGCTGCACCCAACGAAAGCGGACGCAACA | |
| | TCCTCCGATGGCAGCTG | 4651 |
| | CAGCTGCCATCGGAGGA | 4652 |
| Porphyric Herbicide | TCCTTCGTCCACTTTCAGATGTCGCCGCAGAATCTCTTTCAAAATT | 4653 |
| Resistant | TCATTATCCACCAATGGCAGCTGTGTCACTTTCCTATCCTAAAGAA | |
| PPO | GCAATTAGATCAGAGTGCTTGATTGACGG | |
| Spinacia oleracea | CCGTCAATCAAGCACTCTGATCTAATTGCTTCTTTAGGATAGGAAA | 4654 |
| Val390Met | GTGACACAGCTGCCATTGGTGGATAATGAAATTTTGAAAGAGATT | |
| GTT-ATG | CTGCGGCGACATCTGAAAGTGGACGAAGGA | |
| | TCCACCAATGGCAGCTG | 4655 |
| | CAGCTGCCATTGGTGGA | 4656 |
| Porphyric Herbicide | TTTTGCGTCCACTTTCAAGCGATGCTGCAGATGCTCTATCAAGATT | 4657 |
| Resistant | CTATTATCCACCGATGGCTGCTGTAACTGTTTCGTATCCAAAGGAA | |
| Zea mays | CCATCAATTAAGCATTCTTTTCTAATTGCTTCCTTTGGATACGAAAC | 4658 |
| Val363Met | AGTTACAGCAGCCATCGGTGGATAATAGAATCTTGATAGAGCATC | |
| GTT-ATG | TGCAGCATCGCTTGAAAGTGGACGCAAAA | |
| | TCCACCGATGGCTGCTG | 4659 |
| | CAGCAGCCATCGGTGGA | 4660 |
| Porphyric Herbicide | TCTTGCGGCCACTTTCAAGTGATGCAGCAGATGCTCTGTCAATATT | 4661 |
| Resistant | CTATTATCCACCAATGGCTGCTGTAACTGTTTCATATCCAAAAGAA | |
| PPO | GCAATTAGAAAAGAATGCTTAATTGACGG | |
| Oryza sativa | CCGTCAATTAAGCATTCTTTTCTAATTGCTTCTTTTGGATATGAAAC | 4662 |
| Val364Met | AGTTACAGCAGCCATTGGTGGATAATAGAATATTGACAGAGCATC | |
| GTT-ATG | TGCTGCATCACTTGAAAGTGGCCGCAAGA | |
| | TCCACCAATGGCTGCTG | 4663 |
| | CAGCAGCCATTGGTGGA | 4664 |
| Porphyric Herbicide | CTGGTCAAGGAGCAGGCGCCCGCCGCCGCCGAGGCCCTGGGCT | 4665 |
| Resistant | CCTTCGACTACCCGCCGATGGGCGCCGTGACGCTGTCGTACCCG | |
| PPO | CTGAGCGCCGTGCGGGAGGAGCGCAAGGCCTCGG | |
| Chlamydomonas | CCGAGGCCTTGCGCTCCTCCCGCACGGCGCTCAGCGGGTACGAC | 4666 |
| reinhardtii | AGCGTCACGGCGCCCATCGGCGGGTAGTCGAAGGAGCCCAGGG | |
| Val389Met | CCTCGGCGGCGGCGGGCGCCTGCTCCTTGACCAG | |
| GTG-ATG | ACCCGCCGATGGGCGCC | 4667 |
| | GGCGCCCATCGGCGGGT | 4668 |

TABLE 31

Genome-Altering Oligos Conferring
Triazine Resistance

| | |
|---|---|
| Phenotype, Gene, | SEQ |

| | | |
|---|---|---|
| Plant & Targeted | | ID |
| Alteration | Altering Oligos | NO: |

| | | |
|---|---|---|
| Triazine Resistant | AAACTTACAACATTGTAGCTGCTCACGGTTATTTTGGCCGATTGAT | 4669 |
| D1 Protein | TTTCCAATATGCTACTTTCAACAATTCTCGTTCTTTACATTTCTTCTT | |
| Arabidopsis thaliana | AGCGGCTTGGCCGGTAGTAGGTATTTG | |
| Ser264Thr | CAAATACCTACTACCGGCCAAGCCGCTAAGAAGAAATGTAAAGAA | 4670 |
| AGT-ACT | CGAGAATTGTTGAAAGTAGCATATTGGAAAATCAATCGGCCAAAAT | |
| | AACCGTGAGCAGCTACAATGTTGTAAGTTT | |
| | ATATGCTACTTTCAACA | 4671 |
| | TGTTGAAAGTAGCATAT | 4672 |
| Triazine Resistant | AAACTTATAACATCGTAGCCGCTCATGGTTATTTTGGCCGATTGAT | 4673 |
| D1 Protein | CTTCCAATATGCTACTTTCAACAACTCTCGTTCGTTACACTTCTTCC | |
| Nicotiana tabacum | TAGCTGCTTGGCCTGTAGTAGGTATCTG | |
| Ser264Thr | CAGATACCTACTACAGGCCAAGCAGCTAGGAAGAAGTGTAACGAA | 4674 |
| AGT-ACT | CGAGAGTTGTTGAAAGTAGCATATTGGAAGATCAATCGGCCAAAA | |
| | TAACCATGAGCGGCTACGATGTTATAAGTTT | |
| | ATATGCTACTTTCAACA | 4675 |
| | TGTTGAAAGTAGCATAT | 4676 |
| Triazine Resistant | AAACTTATAATATCGTAGCCGCTCATGGTTATTTTGGCCGATTGAT | 4677 |
| D1 Protein | CTTCCAATATGCTACTTTTAACAACTCTCGCTCTTTACATTTCTTCT | |
| Populus deltoides | TAGCTGCTTGGCCTGTAGTAGGTATCTG | |
| Ser264Thr | CAGATACCTACTACAGGCCAAGCAGCTAAGAAGAAATGTAAAGAG | 4678 |
| AGT-ACT | CGAGAGTTGTTAAAAGTAGCATATTGGAAGATCAATCGGCCAAAA | |
| | TAACCATGAGCGGCTACGATATTATAAGTTT | |
| | ATATGCTACTTTTAACA | 4679 |
| | TGTTAAAAGTAGCATAT | 4680 |
| Triazine Resistant | AAACTTATAATATCGTAGCCGCTCATGGTTATTTTGGCCGATTGAT | 4681 |
| D1 Protein | CTTCCAATATGCTACTTTCAACAACTCTCGTTCGTTACACTTCTTCC | |
| Petunia x hybrida | TAGCTGCTTGGCCTGTAGTAGGTATCTG | |
| Ser264Thr | CAGATACCTACTACAGGCCAAGCAGCTAGGAAGAAGTGTAACGAA | 4682 |
| AGT-ACT | CGAGAGTTGTTGAAAGTAGCATATTGGAAGATCAATCGGCCAAAA | |
| | TAACCATGAGCGGCTACGATATTATAAGTTT | |
| | ATATGCTACTTTCAACA | 4683 |
| | TGTTGAAAGTAGCATAT | 4684 |
| Triazine Resistant | AAACTTATAATATCGTAGCTGCTCATGGTTATTTTGGCCGATTGAT | 4685 |
| D1 Protein | CTTCCAATATGCTACTTTCAACAATTCTCGTTCTTTACATTTCTTCC | |
| Magnolia pyramidata | TAGCTGCTTGGCCTGTAGTAGGTATCTG | |
| Ser264Thr | CAGATACCTACTACAGGCCAAGCAGCTAGGAAGAAATGTAAAGAA | 4686 |
| AGT-ACT | CGAGAATTGTTGAAAGTAGCATATTGGAAGATCAATCGGCCAAAA | |
| | TAACCATGAGCAGCTACGATATTATAAGTTT | |
| | ATATGCTACTTTCAACA | 4687 |
| | TGTTGAAAGTAGCATAT | 4688 |
| Triazine Resistant | AAACCTATAATATTGTAGCAGCTCATGGTTATTTTGGCCGATTGAT | 4689 |
| D1 Protein | CTTCCAATATGCTACTTTCAACAACTCTCGTTCTTTACATTTCTTCC | |
| Medicago sativa | TAGCTGCTTGGCCTGTAGTAGGTATCTG | |
| Ser264Thr | CAGATACCTACTACAGGCCAAGCAGCTAGGAAGAAATGTAAAGAA | 4690 |
| AGT-ACT | CGAGAGTTGTTGAAAGTAGCATATTGGAAGATCAATCGGCCAAAA | |
| | TAACCATGAGCTGCTACAATATTATAGGTTT | |
| | ATATGCTACTTTCAACA | 4691 |
| | TGTTGAAAGTAGCATAT | 4692 |
| Triazine Resistant | AAACCTATAATATTGTAGCTGCTCATGGTTATTTTGGCCGATTGAT | 4693 |
| D1 Protein | CTTCCAATATGCAACTTTCAACAATTCTCGTTCTTTACATTTCTTCT | |
| Glycine max | TAGCTGCTTGGCCTGTAGTAGGTATTTG | |
| Ser264Thr | CAAATACCTACTACAGGCCAAGCAGCTAAGAAGAAATGTAAAGAA | 4694 |
| AGT-ACT | CGAGAATTGTTGAAAGTTGCATATTGGAAGATCAATCGGCCAAAA | |
| | TAACCATGAGCAGCTACAATATTATAGGTTT | |
| | ATATGCAACTTTCAACA | 4695 |
| | TGTTGAAAGTTGCATAT | 4696 |
| Triazine Resistant | AAACTTACAACATTGTAGCTGCTCACGGTTATTTTGGCCGATTGAT | 4697 |
| D1 Protein | CTTCCAATATGCTACTTTCAACAATTCTCGTTCTTTACATTTCTTCT | |
| Brassica napus | TAGCGGCTTGGCCGGTAGTAGGTATTTG | |
| Gly264Thr | CAAATACCTACTACCGGCCAAGCCGCTAAGAAGAAATGTAAAGAA | 4698 |
| GGT-ACT | CGAGAATTGTTGAAAGTAGCATATTGGAAGATCAATCGGCCAAAA | |
| | TAACCGTGAGCAGCTACAATGTTGTAAGTTT | |
| | ATATGCTACTTTCAACA | 4699 |
| | TGTTGAAAGTAGCATAT | 4700 |
| Triazine Resistant | AAACTTATAATATTGTGGCCGCTCATGGTTATTTTGGCCGATTAAT | 4701 |
| D1 Protein | CTTCCAATATGCTACTTTTAACAACTCTCGTTCTTTACACTTCTTCT | |
| Oryza sativa | TGGCTGCTTGGCCTGTAGTAGGGATTTG | |
| Ser264Thr | CAAATCCCTACTACAGGCCAAGCAGCCAAGAAGAAGTGTAAAGAA | 4702 |
| AGT-ACT | CGAGAGTTGTTAAAAGTAGCATATTGGAAGATTAATCGGCCAAAAT | |
| | AACCATGAGCGGCCACAATATTATAAGTTT | |
| | ATATGCTACTTTTAACA | 4703 |
| | TGTTAAAAGTAGCATAT | 4704 |
| Triazine Resistant | AGACTTATAATATTGTGGCTGCTCACGGTTATTTTGGTCGATTAAT | 4705 |
| D1 Protein | CTTCCAATATGCTACTTTCAACAATTCTCGTTCTTTACACTTCTTCT | |
| Zea mays | TGGCTGCTTGGCCTGTAGTAGGGATCTG | |
| Ser264Thr | CAGATCCCTACTACAGGCCAAGCAGCCAAGAAGAAGTGTAAAGAA | 4706 |
| AGT-ACT | CGAGAATTGTTGAAAGTAGCATATTGGAAGATTAATCGACCAAAAT | |
| | AACCGTGAGCAGCCACAATATTATAAGTCT | |
| | ATATGCTACTTTCAACA | 4707 |
| | TGTTGAAAGTAGCATAT | 4708 |
| Triazine Resistant | AAACTTACAACATTGTAGCTGCTCACGGTTATTTTGGCCGATTGAT | 4709 |
| D1 Protein | TTTCCAATATGCTACTTTCAACAATTCTCGTTCTTTACATTTCTTCTT | |
| Arabidopsis thaliana | AGCGGCTTGGCCGGTAGTAGGTATTTG | |
| Ser264Thr | CAAATACCTACTACCGGCCAAGCCGCTAAGAAGAAATGTAAAGAA | 4710 |
| AGT-ACT | CGAGAATTGTTGAAAGTAGCATATTGGAAAATCAATCGGCCAAAAT | |
| | AACCGTGAGCAGCTACAATGTTGTAAGTTT | |
| | ATATGCTACTTTCAACA | 4711 |
| | TGTTGAAAGTAGCATAT | 4712 |
| Triazine Resistant | AAACTTATAACATCGTAGCCGCTCATGGTTATTTTGGCCGATTGAT | 4713 |
| D1 Protein | CTTCCAATATGCTACTTTCAACAACTCTCGTTCGTTACACTTCTTCC | |
| Nicotiana tabacum | TAGCTGCTTGGCCTGTAGTAGGTATCTG | |
| Ser264Thr | CAGATACCTACTACAGGCCAAGCAGCTAGGAAGAAGTGTAACGAA | 4714 |
| AGT-ACT | CGAGAGTTGTTGAAAGTAGCATATTGGAAGATCAATCGGCCAAAA | |
| | TAACCATGAGCGGCTACGATGTTATAAGTTT | |
| | ATATGCTACTTTCAACA | 4715 |
| | TGTTGAAAGTAGCATAT | 4716 |
| Triazine Resistant | AAACTTATAATATCGTAGCCGCTCATGGTTATTTTGGCCGATTGAT | 4717 |
| D1 Protein | CTTCCAATATGCTACTTTTAACAACTCTCGCTCTTTACATTTCTTCT | |
| Populus deltoides | TAGCTGCTTGGCCTGTAGTAGGTATCTG | |
| Ser264Thr | CAGATACCTACTACAGGCCAAGCAGCTAAGAAGAAATGTAAAGAG | 4718 |
| AGT-ACT | CGAGAGTTGTTAAAAGTAGCATATTGGAAGATCAATCGGCCAAAA | |
| | TAACCATGAGCGGCTACGATATTATAAGTTT | |
| | ATATGCTACTTTTAACA | 4719 |
| | TGTTAAAAGTAGCATAT | 4720 |
| Triazine Resistant | AAACTTATAATATCGTAGCCGCTCATGGTTATTTTGGCCGATTGAT | 4721 |
| D1 Protein | CTTCCAATATGCTACTTTCAACAACTCTCGTTCGTTACACTTGTTCC | |
| Petunia x hybrida | TAGCTGCTTGGCCTGTAGTAGGTATCTG | |
| Ser264Thr | CAGATACCTACTACAGGCCAAGCAGCTAGGAAGAAGTGTAACGAA | 4722 |
| AGT-ACT | CGAGAGTTGTTGAAAGTAGCATATTGGAAGATCAATCGGCCAAAA | |
| | TAACCATGAGCGGCTACGATATTATAAGTTT | |
| | ATATGCTACTTTCAACA | 4723 |
| | TGTTGAAAGTAGCATAT | 4724 |
| Triazine Resistant | AAACTTATAATATCGTAGCTGCTCATGGTTATTTTGGCCGATTGAT | 4725 |
| D1 Protein | CTTCCAATATGCTACTTTCAACAATTCTCGTTCTTTACATTTCTTCC | |
| Magnolia pyramidata | TAGCTGCTTGGCCTGTAGTAGGTATCTG | |
| Ser264Thr | CAGATACCTACTACAGGCCAAGCAGCTAGGAAGAAATGTATAAGAA | 4726 |
| AGT-ACT | CGAGAATTGTTGAAAGTAGCATATTGGAAGATCAATCGGCCAAAA | |
| | TAACCATGAGCAGCTACGATATTATAAGTTT | |
| | ATATGCTACTTTCAACA | 4727 |
| | TGTTGAAAGTAGCATAT | 4728 |
| Triazine Resistant | AAACCTATAATATTGTAGCAGCTCATGGTTATTTTGGCCGATTGAT | 4729 |
| D1 Protein | CTTCCAATATGCTACTTTCAACAACTCTCGTTCTTTACATTTCTTCC | |
| Medicago sativa | TAGCTGCTTGGCCTGTAGTAGGTATCTG | |
| Ser264Thr | CAGATACCTACTACAGGCCAAGCAGCTAGGAAGAAATGTAAAGAA | 4730 |
| AGT-ACT | CGAGAGTTGTTGAAAGTAGCATATTGGAAGATCAATCGGCCAAAA | |
| | TAACCATGAGCTGCTACAATATTATAGGTTT | |
| | ATATGCTACTTTCAACA | 4731 |
| | TGTTGAAAGTAGCATAT | 4732 |
| Triazine Resistant | AAACCTATAATATTGTAGCTGCTCATGGTTATTTTGGCCGATTGAT | 4733 |
| D1 Protein | CTTCCAATATGCAACTTTCAACAATTCTCGTTCTTTACATTTCTTCT | |
| Glycine max | TAGCTGCTTGGCCTGTAGTAGGTATTTG | |
| Ser264Thr | CAAATACCTACTACAGGCCAAGCAGCTAAGAAGAAATGTAAAGAA | 4734 |
| AGT-ACT | CGAGAATTGTTGAAAGTTGCATATTGGAAGATCAATCGGCCAAAA | |
| | TAACCATGAGCAGCTACAATATTATAGGTTT | |
| | ATATGCAACTTTCAACA | 4735 |
| | TGTTGAAAGTTGCATAT | 4736 |
| Triazine Resistant | AAACTTACAACATTGTAGCTGCTCACGGTTATTTTGGCCGATTGAT | 4737 |
| D1 Protein | CTTCCAATATGCTACTTTCAACAATTCTCGTTCTTTACATTTCTTCT | |
| Brassica napus | TAGCGGCTTGGCCGGTAGTAGGTATTTG | |
| Gly264Thr | CAAATACCTACTACCGGCCAAGCCGCTAAGAAGAAATGTAAAGAA | 4738 |
| GGT-ACT | CGAGAATTGTTGAAAGTAGCATATTGGAAGATCAATCGGCCAAAA | |
| | TAACCGTGAGCAGCTACAATGTTGTAAGTTT | |
| | ATATGCTACTTTCAACA | 4739 |
| | TGTTGAAAGTAGCATAT | 4740 |
| Triazine Resistant | AAACTTATAATATTGTGGCCGCTCATGGTTATTTTGGCCGATTAAT | 4741 |
| D1 Protein | CTTCCAATATGCTACTTTTAACAACTCTCGTTCTTTACACTTCTTCT | |
| Oryza sativa | TGGCTGCTTGGCCTGTAGTAGGGATTTG | |
| Ser264Thr | CAAATCCCTACTACAGGCCAAGCAGCCAAGAAGAAGTGTAAAGAA | 4742 |
| AGT-ACT | CGAGAGTTGTTAAAAGTAGCATATTGGAAGATTAATCGGCCAAAAT | |
| | AACCATGAGCGGCCACAATATTATAAGTTT | |
| | ATATGCTACTTTTAACA | 4743 |
| | TGTTAAAAGTAGCATAT | 4744 |
| Triazine Resistant | AGACTTATAATATTGTGGCTGCTCACGGTTATTTTGGTCGATTAAT | 4745 |
| D1 Protein | CTTCCAATATGCTACTTTCAACAATTCTCGTTCTTTACACTTCTTCT | |
| Zea mays | TGGCTGCTTGGCCTGTAGTAGGGATCTG | |
| Ser264Thr | CAGATCCCTACTACAGGCCAAGCAGCCAAGAAGAAGTGTAAAGAA | 4746 |
| AGT-ACT | CGAGAATTGTTGAAAGTAGCATATTGGAAGATTAATCGACCAAAAT | |
| | AACCGTGAGCAGCCACAATATTATAAGTCT | |
| | ATATGCTACTTTCAACA | 4747 |
| | TGTTGAAAGTAGCATAT | 4748 |
| Triazine Resistant | AAACTTACAACATTGTAGCTGCTCACGGTTATTTTGGCCGATTGAT | 4749 |
| D1 Protein | TTTCCAATATGCTACTTTCAACAATTCTCGTTCTTTACATTTCTTCTT | |
| Arabidopsis thaliana | AGCGGCTTGGCCGGTAGTAGGTATTTG | |
| Ser264Thr | CAAATACCTACTACCGGCCAAGCCGCTAAGAAGAAATGTAAAGAA | 4750 |
| AGT-ACT | CGAGAATTGTTGAAAGTAGCATATTGGAAAATCAATCGGCCAAAAT | |
| | AACCGTGAGCAGCTACAATGTTGTAAGTTT | |
| | ATATGCTACTTTCAACA | 4751 |
| | TGTTGAAAGTAGCATAT | 4752 |
| Triazine Resistant | AAACCTACAATATTGTGGCTGCTCACGGTTATTTCGGCCGATTGAT | 4753 |
| D1 Protein | CTTCCAGTATGCTACTTTCAACAACTCCCGTTCTTTACATTTCTTCT | |
| Picea abies | TAGCTGCTTGGCCCGTAGCAGGTATCTG | |
| Ser264Thr | CAGATACCTGCTACGGGCCAAGCAGCTAAGAAGAAATGTAAAGAA | 4754 |
| AGT-ACT | CGGGAGTTGTTGAAAGTAGCATACTGGAAGATCAATCGGCCGAAA | |
| | TAACCGTGAGCAGCCACAATATTGTAGGTTT | |
| | GTATGCTACTTTCAACA | 4755 |
| | TGTTGAAAGTAGCATAC | 4756 |
| Triazine Resistant | AAACCTATAATATTGTAGCTGCTCACGGTTATTTTGGCCGATTGAT | 4757 |
| D1 Protein | CTTCCAATATGCTACTTTCAACAATTCTCGCTCTTTACATTTCTTCC | |
| Vicia faba | TAGCTGCTTGGCCTGTAGTAGGTATCTG | |
| Ser264Thr | CAGATACCTACTACAGGCCAAGCAGCTAGGAAGAAATGTAAAGAG | 4758 |
| AGT-ACT | CGAGAATTGTTGAAAGTAGCATATTGGAAGATCAATCGGCCAAAA | |
| | TAACCGTGAGCAGCTACAATATTATAGGTTT | |
| | ATATGCTACTTTCAACA | 4759 |
| | TGTTGAAAGTAGCATAT | 4760 |
| Triazine Resistant | AGACTTATAATATTGTGGCTGCTCATGGTTATTTTGGCCGATTAAT | 4761 |
| D1 Protein | CTTCCAATATGCTACTTTCAACAACTCTCGTTCTTTACACTTCTTCT | |
| Hordeum vulgare | TGGCTGCTTGGCCTGTAGTAGGAATCTG | |
| Ser264Thr | CAGATTCCTACTACAGGCCAAGCAGCCAAGAAGAAGTGTAAAGAA | 4762 |
| AGT-ACT | CGAGAGTTGTTGAAAGTAGCATATTGGAAGATTAATCGGCCAAAA | |
| | TAACCATGAGCAGCCACAATATTATAAGTCT | |
| | ATATGCTACTTTCAACA | 4763 |
| | TGTTGAAAGTAGCATAT | 4764 |
| Triazine Resistant | AAACTTATAATATTGTGGCTGCTCATGGTTATTTTGGCCGATTAAT | 4765 |
| D1 Protein | CTTCCAATATGCTACTTTCAACAACTCTCGTTCTTTACACTTCTTCT | |
| Triticum aestivum | TGGCTGCTTGGCCTGTAGTAGGAATCTG | |
| Ser264Thr | CAGATTCCTACTACAGGCCAAGCAGCCAAGAAGAAGTGTAAAGAA | 4766 |
| AGT-ACT | CGAGAGTTGTTGAAAGTAGCATATTGGAAGATTAATCGGCCAAAA | |
| | TAACCATGAGCAGCCACAATATTATAAGTTT | |
| | ATATGCTACTTTCAACA | 4767 |
| | TGTTGAAAGTAGCATAT | 4768 |
| Triazine Resistant | AAACTTATAATATTGTAGCTGCTCATGGTTATTTTGGCCGATTAATC | 4769 |
| D1 Protein | TTCCAATATGCAACTTTCAACAATTCTCGTTCTTTACATTTCTTCCT | |
| Vigna unguiculata | AGCTGCTTGGCCTGTAGTAGGTATTTG | |
| Ser264Thr | CAAATACCTACTACAGGCCAAGCAGCTAGGAAGAAATGTAAAGAA | 4770 |
| AGT-ACT | CGAGAATTGTTGAAAGTTGCATATTGGAAGATTAATCGGCCAAAAT | |
| | AACCATGAGCAGCTACAATATTATAAGTTT | |
| | ATATGCAACTTTCAACA | 4771 |
| | TGTTGAAAGTTGCATAT | 4772 |
| Triazine Resistant | AAACCTATAATATTGTAGCTGCTCACGGTTATTTTGGCCGATTGAT | 4773 |
| D1 Protein | CTTCCAATATGCAACTTTCAACAACTCTCGTTCTTTACACTTCTTCT | |
| Lotus japonicus | TAGCTGCTTGGCCTGTTGTAGGTATCTG | |
| Ser264Thr | CAGATACCTACAACAGGCCAAGCAGCTAAGAAGAAGTGTAAAGAA | 4774 |
| AGT-ACT | CGAGAGTTGTTGAAAGTTGCATATTGGAAGATCAATCGGCCAAAA | |
| | TAACCGTGAGCAGCTACAATATTATAGGTTT | |
| | ATATGCAACTTTCAACA | 4775 |
| | TGTTGAAAGTTGCATAT | 4776 |
| Triazine Resistant | AAACTTACAACATTGTAGCTGCTCACGGTTATTTTGGCCGATTGAT | 4777 |
| D1 Protein | CTTCCAATATGCTACTTTCAACAATTCTCGTTCTTTACATTTCTTCT | |
| Sinapis alba | TAGCGGCTTGGCCGGTAGTAGGTATTTG | |
| Ser264Thr | CAAATACCTACTACCGGCCAAGCCGCTAAGAAGAAATGTAAAGAA | 4778 |
| AGT-ACT | CGAGAATTGTTGAAAGTAGCATATTGGAAGATCAATCGGCCAAAA | |
| | TAACCGTGAGCAGCTACAATGTTGTAAGTTT | |
| | ATATGCTACTTTCAACA | 4779 |
| | TGTTGAAAGTAGCATAT | 4780 |
| Triazine Resistant | AAACCTATAATATTGTAGCTGCTCACGGTTATTTTGGCCGATTGAT | 4781 |
| D1 Protein | CTTCCAATATGCTACTTTCAACAATTCTCGCTCTTTACATTTCTTCC | |
| Pisum sativum | TAGCTGCTTGGCCTGTAGTAGGTATCTG | |
| Ser264Thr | CAGATACCTACTACAGGCCAAGCAGCTAGGAAGAAATGTAAAGAG | 4782 |
| AGT-ACT | CGAGAATTGTTGAAAGTAGCATATTGGAAGATCAATCGGCCAAAA | |
| | TAACCGTGAGCAGCTACAATATTATAGGTTT | |
| | ATATGCTACTTTCAACA | 4783 |
| | TGTTGAAAGTAGCATAT | 4784 |
| Triazine Resistant | AAACTTATAATATCGTAGCTGCTCATGGTTATTTTGGTCGATTGAT | 4785 |
| D1 Protein | CTTCCAATATGCTACTTTCAACAACTCTCGTTCTTTACACTTCTTCT | |
| Spinacia oleracea | TAGCTGCTTGGCCTGTAGTAGGTATTTG | |
| Ser264Thr | CAAATACCTACTACAGGCCAAGCAGCTAAGAAGAAGTGTAAAGAA | 4786 |
| AGT-ACT | CGAGAGTTGTTGAAAGTAGCATATTGGAAGATCAATCGACCAAAA | |
| | TAACCATGAGCAGCTACGATATTATAAGTTT | |
| | ATATGCTACTTTCAACA | 4787 |
| | TGTTGAAAGTAGCATAT | 4788 |
| Triazine Resistant | AAACTTATAACATCGTAGCCGCTCATGGTTATTTTGGCCGATTGAT | 4789 |
| D1 Protein | CTTCCAATATGCTACTTTCAACAACTCTCGTTCGTTACACTTCTTCC | |
| Nicotiana debneyi | TAGCTGCTTGGCCTGTAGTAGGTATCTG | |
| Ser264Thr | CAGATACCTACTACAGGCCAAGCAGCTAGGAAGAAGTGTAACGAA | 4790 |
| AGT-ACT | CGAGAGTTGTTGAAAGTAGCATATTGGAAGATCAATCGGCCAAAA | |
| | TAACCATGAGCGGCTACGATGTTATAAGTTT | |
| | ATATGCTACTTTCAACA | 4791 |
| | TGTTGAAAGTAGCATAT | 4792 |
| Triazine Resistant | AAACTTATAATATCGTAGCCGCTCATGGTTATTTTGGCCGATTGAT | 4793 |
| D1 Protein | CTTCCAATATGCTACTTTCAACAACTCTCGTTCGTTACACTTCTTCC | |
| Solanum nigrum | TAGCTGCTTGGCCTGTAGTAGGTATCTG | |
| Ser264Thr | CAGATACCTACTACAGGCCAAGCAGCTAGGAAGAAGTGTAACGAA | 4794 |
| AGT-ACT | CGAGAGTTGTTGAAAGTAGCATATTGGAAGATCAATCGGCCAAAA | |
| | TAACCATGAGCGGCTACGATATTATAAGTTT | |
| | ATATGCTACTTTCAACA | 4795 |
| | TGTTGAAAGTAGCATAT | 4796 |
| Triazine Resistant | AAACTTATAACATCGTAGCCGCTCATGGTTATTTTGGCCGATTGAT | 4797 |
| D1 Protein | CTTCCAATATGCTACTTTCAACAACTCTCGTTCGTTACACTTCTTCC | |
| Nicotiana | TAGCTGCTTGGCCTGTAGTAGGTATCTG | |
| plumbaginifolia | CAGATACCTACTACAGGCCAAGCAGCTAGGAAGAAGTGTAACGAA | 4798 |
| Ser264Thr | CGAGAGTTGTTGAAAGTAGCATATTGGAAGATCAATCGGCCAAAA | |
| AGT-ACT | TAACCATGAGCGGCTACGATGTTATAAGTTT | |
| | ATATGCTACTTTCAACA | 4799 |
| | TGTTGAAAGTAGCATAT | 4800 |

### EXAMPLE 27

### Engineering Male- or Female-Sterile Plants

Flower development in distantly related dicot plant species is increasingly better understood and appears to be regulated by a family of genes which encode regulatory proteins. These genes include, for example, AGAMOUS (AG), APETALA1 (AP1), and APETALA3 (AP3) and PISTILLATA (PI) in Arabidopsis thaliana, and DEFICIENS A (DEFA), GLOBOSA (GLO), SQUAMOSA (SQUA), and PLENA (PLE) in Antirrhinum majus. Genetic studies have shown that the DEFA, GLO and AP3 genes are essential for petal and stamen development. Sequence analysis of these genes revealed that the gene products contain a conserved MADS box region, a DNA-binding domain. Using these clones as probes, MADS box genes have also been isolated from other species including tomato, tobacco, petunia, Brassica napus, and maize.

Altering the expression of these genes results in altered floral morphology. For example, mutations in AP3 and PI result in male-sterile flowers because petals develop in place of stamens.

The attached tables disclose exemplary oligonucleotide base sequences which can be used to generate site-specific mutations that confer altered floral structures in plants.

TABLE 32

Oligonucleotides to produce male-sterile plants

| | |
|---|---|
| Phenotype, Gene, | SEQ |

| | | |
|---|---|---|
| Plant & Targeted | | ID |
| Alteration | Altering Oligos | NO: |

| | | |
|---|---|---|
| Male-sterile | TTGTCCTCTCCACCAAATCTCTTCAACAAAAAGATTAAACAAAGAG | 4801 |
| AP3 | AGAAGAATATGGCGTGAGGGAAGATCCAGATCAAGAGGATAGAGA | |
| Arabidopsis thaliana | ACCAGACAAACAGACAAGTGACGTATTCAA | |
| Arg3Term | TTGAATACGTCACTTGTCTGTTTGTCTGGTTCTCTATCCTCTTGATC | 4802 |
| AGA-TGA | TGGATCTTCCCTCACGCCATATTCTTCTCTCTTTGTTTAATCTTTTT | |
| | GTTGAAGAGATTTGGTGGAGAGGACAA | |
| | ATATGGCGTGAGGGAAG | 4803 |
| | CTTCCCTCACGCCATAT | 4804 |
| Male-sterile | TCTCCACCAAATCTCTTCAACAAAAAGATTAAACAAAGAGAGAAGA | 4805 |
| AP3 | ATATGGCGAGAGGGTAGATCCAGATCAAGAGGATAGAGAACCAGA | |
| Arabidopsis thaliana | CAAACAGACAAGTGACGTATTCAAAGAGAA | |
| Lys5Term | TTCTCTTTGAATACGTCACTTGTCTGTTTGTCTGGTTCTCTATCCTC | 4806 |
| AAG-TAG | TTGATCTGGATCTACCCTCTCGCCATATTCTTCTCTCTTTGTTTAAT | |
| | CTTTTTGTTGAAGAGATTTGGTGGAGA | |
| | CGAGAGGGTAGATCCAG | 4807 |
| | CTGGATCTACCCTCTCG | 4808 |
| Male-sterile | CCAAATCTCTTCAACAAAAAGATTAAACAAAGAGAGAAGAATATGG | 4809 |
| AP3 | CGAGAGGGAAGATCTAGATCAAGAGGATAGAGAACCAGACAAACA | |
| Arabidopsis thaliana | GACAAGTGACGTATTCAAAGAGAAGGAATG | |
| Gln7Term | CATTCCTTCTCTTTGAATACGTCACTTGTCTGTTTGTCTGGTTCTCT | 4810 |
| GAG-TAG | ATCCTCTTGATCTAGATCTTCCCTCTCGCCATATTCTTCTCTCTTTG | |
| | TTTAATCTTTTTGTTGAAGAGATTTGG | |
| | GGAAGATCTAGATCAAG | 4811 |
| | CTTGATCTAGATCTTCC | 4812 |
| Male-sterile | CTCTTCAACAAAAAGATTAAACAAAGAGAGAAGAATATGGCGAGAG | 4813 |
| AP3 | GGAAGATCCAGATCTAGAGGATAGAGAACCAGACAAACAGACAAG | |
| Arabidopsis thaliana | TGACGTATTCAAAGAGAAGGAATGGTTTAT | |
| Lys9Term | ATAAACCATTCCTTCTCTTTGAATACGTCACTTGTCTGTTTGTCTGG | 4814 |
| AAG-TAG | TTCTCTATCCTCTAGATCTGGATCTTCCCTCTCGCCATATTCTTCTC | |
| | TCTTTGTTTAATCTTTTTGTTGAAGAG | |
| | TCCAGATCTAGAGGATA | 4815 |
| | TATCCTCTAATCTGGA | 4816 |
| Male-sterile | AGAGGGAAGATCCAGATCAAGAGGATAGAGAACCAGACCAACCG | 4817 |
| AP3 | ACAAGTGACGTATTCTTAGAGAAGAAATGGTTTGTTCAAGAAAGCT | |
| Brassica oleracea | CACGAGCTTACAGTTTTATGTGATGCTAGGG | |
| Lys23Term | CCCTAGCATCACATAAAACTGTAAGCTCGTGAGCTTTCTTGAACAA | 4818 |
| AAG-TAG | ACCATTTCTTCTCTAAGAATACGTCACTTGTCGGTTGGTCTGGTTC | |
| | TCTATCCTCTTGATCTGGATCTTCCCTCT | |
| | CGTATTCTTAGAGAAGA | 4819 |
| | TCTTCTCTAAGAATACG | 4820 |
| Male-sterile | GGGAAGATCCAGATCAAGAGGATAGAGAACCAGACCAACCGACAA | 4821 |
| AP3 | GTGACGTATTCTAAGTGAAGAAATGGTTTGTTCAAGAAAGCTCACG | |
| Brassica oleracea | AGCTTACAGTTTTATGTGATGCTAGGGTTT | |
| Arg24Term | AAACCCTAGCATCACATAAAACTGTAAGCTCGTGAGCTTTCTTGAA | 4822 |
| AGA-TGA | CAAACCATTTCTTCACTTAGAATACGTCACTTGTCGGTTGGTCTGG | |
| | TTCTCTATCCTCTTGATCTGGATCTTCCC | |
| | ATTCTAAGTGAAGAAAT | 4823 |
| | ATTTCTTCACTTAGAAT | 4824 |
| Male-sterile | AAGATCCAGATCAAGAGGATAGAGAACCAGACCAACCGAGAAGTG | 4825 |
| AP3 | ACGTATTCTAAGAGATGAAATGGTTTGTTCAAGAAAGCTCACGAGC | |
| Brassica oleracea | TTACAGTTTTATGTGATGCTAGGGTTTCGA | |
| Arg25Term | TCGAAACCCTAGCATCACATAAAACTGTAAGCTCGTGAGCTTTCTT | 4826 |
| AGA-TGA | GAACAAACCATTTCACTCTTAGAATACGTCACTTGTCGGTTGGTC | |
| | TGGTTCTCTATCCTCTTGATCTGGATCTT | |
| | CTAAGAGATGAAATGGT | 4827 |
| | ACCATTTCATCTCTTAG | 4828 |
| Male-sterile | TCAAGAGGATAGAGAACCAGACCAACCGACAAGTGACGTATTCTA | 4829 |
| AP3 | AGAGAAGAAATGGTTAGTTCAAGAAAGCTCACGAGCTTACAGTTTT | |
| Brassica oleracea | ATGTGATGCTAGGGTTTCGATTATCATGTT | |
| Leu28Term | AACATGATAATCGAAACCCTAGCATCACATAAAACTGTTAAGCTCGT | 4830 |
| TTG-TAG | GAGCTTTCTTGAACTAACCATTTCTTCTCTTAGAATACGTCACTTGT | |
| | CGGTTGGTCTGGTTCTCTATCCTCTTGA | |
| | AAATGGTTAGTTCAAGA | 4831 |
| | TCTTGAACTAACCATTT | 4832 |
| Male-sterile | GGCTCGAGGGAAGATCCAGATTAAGAGGATAGAGAACCAAACAAA | 4833 |
| AP3 | CAGGCAGGTCACCTAGTCCAAGAGAAGAAATGGTTTGTTCAAGAA | |
| Brassica napus | AGCACACGAGCTCTCTGTTCTCTGTGATGCT | |
| Tyr21Term | AGCATCACAGAGAACAGAGAGCTCGTGTGCTTTCTTGAACAAACC | 4834 |
| TAC-TAG | ATTTCTTCTCTTGGACTAGGTGACCTGCCTGTTTGTTTGGTTCTCTA | |
| | TCCTCTTAATCTGGATCTTCCCTCGAGCC | |
| | GTCACCTAGTCCAAGAG | 4835 |
| | CTCTTGGACTAGGTGAC | 4836 |
| Male-sterile | CGAGGGAAGATCCAGATTAAGAGGATAGAGAACCAAACAAACAGG | 4837 |
| AP3 | CAGGTCACCTACTCCTAGAGAAGAAATGGTTTGTTCAAGAAAGCAC | |
| Brassica napus | ACGAGCTCTCTGTTCTCTGTGATGCTAAAG | |
| Lys23Term | CTTTAGCATCACAGAGAACAGAGAGCTCGTGTGCTTTCTTGAACAA | 4838 |
| AAG-TAG | ACCATTTCTTCTCTAGGAGTAGGTGACCTGCCTGTTTGTTTGGTTC | |
| | TCTATCCTCTTAATCTGGATCTTCCCTCG | |
| | CCTACTCCTAGAGAAGA | 4839 |
| | TCTTCTCTAGGAGTAGG | 4840 |
| Male-sterile | GGGAAGATCCAGATTAAGAGGATAGAGAACCAAACAAACAGGCAG | 4841 |
| AP3 | GTCACCTACTCCAAGTGAAGAAATGGTTTGTTCAAGAAAGCACACG | |
| Brassica napus | AGCTCTCTGTTCTCTGTGATGCTAAAGTTT | |
| Arg24Term | AAACTTTAGCATCACAGAGAACAGAGAGCTCGTGTGCTTTCTTGAA | 4842 |
| AGA-TGA | CAAACCATTTCTTCACTTGGAGTAGGTGACCTGCCTGTTTGTTTGG | |
| | TTCTCTATCCTCTTAATCTGGATCTTCCC | |
| | ACTCCAAGTGAAGAAAT | 4843 |
| | ATTTCTTCACTTGGAGT | 4844 |
| Male-sterile | AAGATCCAGATTAAGAGGATAGAGAACCAAACAAACAGGCAGGTC | 4845 |
| AP3 | ACCTACTCCAAGAGATGAAATGGTTTGTTCAAGAAAGCACACGAG | |
| Brassica napus | CTCTCTGTTCTCTGTGATGCTAA4GTTTCCA | |
| Arg25Term | TGGAAACTTTAGCATCACAGAGAACAGAGAGCTCGTGTGCTTTCTT | 4846 |
| AGA-TGA | GAACAAACCATTTCATCTCTTGGAGTAGGTGACCTGCCTGTTTGTT | |
| | TGGTTCTCTATCCTCTTAATCTGGATCTT | |
| | CCAAGAGATGAAATGGT | 4847 |
| | ACCATTTCATCTCTTGG | 4848 |
| Male-sterile | GGAGAGAAAGGAAAGCTGGAAGAAGAAAACAAGAGCAGTAGTGG | 4849 |
| DEFA | TAGTGGTTCGATGGCTTGAGGGAAGATCCAGATTAAGAGGATAGA | |
| Antirrhinum majus | GAACCAAACAAACAGGCAGGTCACCTACTCCA | |
| Arg3Term | TGGAGTAGGTGACCTGCCTGTTTGTTTGGTTCTCTATCCTCTTAAT | 4850 |
| CGA-TGA | CTGGATCTTCCCTCAAGCCATCGAACCACTACCACTACTGCTCTTG | |
| | TTTTCTTCTTCCAGCTTTCCTTTCTCTCC | |
| | CGATGGCTTGAGGGAAG | 4851 |
| | CTTCCCTCAAGCCATCG | 4852 |
| Male-sterile | AAAGGAAAGCTGGAAGAAGAAAACAAGAGCAGTAGTGGTAGTGGT | 4853 |
| DEFA | TCGATGGCTCGAGGGTAGATCCAGATTAAGAGGATAGAGAACCAA | |
| Antirrhinum majus | ACAAACAGGCAGGTCACCTACTCCAAGAGAA | |
| Lys5Term | TTCTCTTGGAGTAGGTGACCTGCCTGTTTGTTTGGTTCTCTATCCT | 4854 |
| AAG-TAG | CTTAATCTGGATCTACCCTCGAGCCATCGAACCACTACCACTACTG | |
| | CTCTTGTTTTCTTCTTCCAGCTTTCCTTT | |
| | CTCGAGGGTAGATCCAG | 4855 |
| | CTGGATCTACCCTCGAG | 4856 |
| Male-sterile | AAGCTGGAAGAAGAAAACAAGAGCAGTAGTGGTAGTGGTTCGATG | 4857 |
| DEFA | GCTCGAGGGAAGATCTAGATTAAGAGGATAGAGAACCAAACAAAC | |
| Antirrhinum majus | AGGCAGGTCACCTACTCCAAGAGAAGAAATG | |
| Gln7Term | CATTTCTTCTCTTGGAGTAGGTGACCTGCCTGTTTGTTTGGTTCTC | 4858 |
| CAG-TAG | TATCCTCTTAATCTAGATCTTCCCTCGAGCCATCGAACCACTACCA | |
| | CTACTGCTCTTGTTTTCTTCTTCCAGCTT | |
| | GGAAGATCTAGATTAAG | 4859 |
| | CTTAATCTAGATCTTCC | 4860 |
| Male-sterile | GAAGAAGAAAACAAGAGCAGTAGTGGTAGTGGTTCGATGGCTCGA | 4861 |
| DEFA | GGGAAGATCCAGATTTAGAGGATAGAGAACCAAACAAACAGGCAG | |
| Antirrhinum majus | GTCACCTACTCCAAGAGAAGAAATGGTTTGT | |
| Lys9Term | ACAAACCATTTCTTCTCTTGGAGTAGGTGACCTGCCTGTTTGTTTG | 4862 |
| AAG-TAG | GTTCTCTATCCTCTAAATCTGGATCTTCCCTCGAGCCATCGAACCA | |
| | CTACCACTACTGCTCTTGTTTTCTTCTTC | |
| | TCCAGATTTAGAGGATA | 4863 |
| | TATCCTCTAAATCTGGA | 4864 |
| Male-sterile | TCAGTAATTCTTAAGATCTCAAACTTTGAGCAAAAAGAAAAAAAAAC | 4865 |
| AP3 | TATGGCTCGTGGGTAGATCCAGATCAAGAGAATAGAGAACCAAAC | |
| Nicotiana tabacum | AAACAGACAAGTCACTTATTCTAAGAGAA | |
| Lys5Term | TTCTCTTAGAATAAGTGACTTGTCTGTTTGTTTGGTTCTCTATTCTC | 4866 |
| AAG-TAG | TTGATCTGGATCTACCCACGAGCCATAGTTTTTTTTTCTTTTTGCTC | |
| | AAAGTTTGAGATCTTAAGAATTACTGA | |
| | CTCGTGGGTAGATCCAG | 4867 |
| | CTGGATCTACCCACGAG | 4868 |
| Male-sterile | ATTCTTAAGATCTCAAACTTTGAGCAAAAAGAAAAAAAAACTATGGC | 4869 |
| AP3 | TCGTGGGAAGATCTAGATCAAGAGAATAGAGAACCAAACAAACAG | |
| Nicotiana tabacum | AGAAGTCACTTATTGTAAGAGAAGAAATG | |
| Gln7Term | GATTTCTTGTCTTAGAATAAGTGAGTTGTGTGTTTGTTTGGTTGTGT | 4870 |
| CAG-TAG | ATTCTCTTGATCTAGATCTTCGGAGGAGGGATAGTTTTTTTTTGTTT | |
| | TTGGTCAAAGTTTGAGATGTTAAGAAT | |
| | GGAAGATCTAGATCAAG | 4871 |
| | CTTGATCTAGATCTTCC | 4872 |
| Male-sterile | AAGATGTGAAAGTTTGAGGAAAAAGAAAAAAAAAGTATGGCTGGTG | 4873 |
| AP3 | GGAAGATCGAGATCTAGAGAATAGAGAACGAAAGAAAGAGAGAAG | |
| Nicotiana tabacum | TGAGTTATTGTAAGAGAAGAAATGGAGTTT | |
| Lys9Term | AAAGTGGATTTGTTGTGTTAGAATAAGTGAGTTGTGTGTTTGTTTGG | 4874 |
| AAG-TAG | TTGTGTATTGTCTAGATGTGGATGTTGGCAGGAGGGATAGTTTTTTT | |
| | TTGTTTTTGGTGAAAGTTTGAGATGTT | |
| | TCCAGATCTAGAGAATA | 4875 |
| | TATTCTCTAGATCTGGA | 4876 |
| Male-sterile | ATCTCAAAGTTTGAGCAAAAAGAAAAAAAAAGTATGGGTCGTGGGA | 4877 |
| AP3 | AGATGGAGATGAAGTGAATAGAGAAGGAAAGAAAGAGAGAAGTGA | |
| Nicotiana tabacum | CTTATTCTAAGAGAAGAAATGGAGTTTTGA | |
| Arg10Term | TGAAAAGTGCATTTCTTGTGTTAGAATAAGTGACTTGTGTGTTTGTT | 4878 |
| AGA-TGA | TGGTTCTCTATTCACTTGATCTGGATCTTCCACGAGCCATAGTTT | |
| | TTTTTTCTTTTTGCTGAAAGTTTGAGAT | |
| | AGATGAAGTGAATAGAG | 4879 |
| | CTCTATTCACTTGATCT | 4880 |
| Male-sterile | GGCTCGAGGAAAGATCCAGATCAAGAGAATAGAGAACACAACGAA | 4881 |
| AP3 | GAGAGAAGTAAGTTAGTCAAAACGAAGGGATGGTCTTTTCAAGAAG | |
| Medicago sativa | GCCAATGAGCTCACTGTTCTTTGTGATGCT | |
| Tyr21Term | AGGATGAGAAAGAACAGTGAGCTGATTGGGGTTCTTGAAAAGACGA | 4882 |
| TAG-TAG | TGGGTTGGTTTTGACTAAGTTAGTTGTGTGTTGGTTGTGTTGTGTAT | |
| | TGTGTTGATGTGGATGTTTGGTGGAGGG | |
| | GTAAGTTAGTGAAAAGG | 4883 |
| | GGTTTTGACTAAGTTAG | 4884 |
| Male-sterile | GTGGAGGAAAGATGGAGATGAAGAGAATAGAGAAGAGAAGGAAGA | 4885 |
| AP3 | GAGAAGTAACTTACTGAAAACGAAGGGATGGTCTTTTCAAGAAGG | |
| Medicago sativa | CCAATGAGGTCACTGTTCTTTGTGATGGTAA | |
| Ser22Term | TTAGGATGAGAAAGAAGAGTGAGGTGATTGGGGTTGTTGAAAAGAG | 4886 |
| TCA-TGA | GATGGGTTGGTTTTCAGTAAGTTAGTTGTGTGTTGGTTGTGTTGTGT | |
| | ATTGTGTTGATGTGGATGTTTCGTGGAG | |
| | AAGTTAGTGAAAACGAA | 4887 |
| | TTCGTTTTCAGTAAGTT | 4888 |
| Male-sterile | CGAGGAAAGATCCAGATCAAGAGAATAGAGAACACAACGAACAGA | 4889 |
| AP3 | CAAGTAACTTACTCATAACGAAGGGATGGTCTTTTCAAGAAGGCCA | |
| Medicago sativa | ATGAGCTCACTGTTCTTTGTGATGCTAAGG | |
| Lys23Term | CCTTAGCATCACAAAGAACAGTGAGCTCATTGGCCTTCTTGAAAAG | 4890 |
| AAA-TAA | ACCATCCCTTCGTTATGAGTAAGTTACTTGTCTGTTCGTTGTGTTCT | |
| | CTATTCTCTTGATCTGGATCTTTCCTCG | |
| | CTTACTCATAACGAAGG | 4891 |
| | CCTTCGTTATGAGTAAG | 4892 |
| Male-sterile | GGAAAGATCCAGATCAAGAGAATAGAGAACACAACGAACAGACAA | 4893 |
| AP3 | GTAACTTACTCAAAATGAAGGGATGGTCTTTTCAAGAAGGCCAATG | |
| Medicago sativa | AGCTCACTGTTCTTTGTGATGCTAAGGTTT | |
| Arg24Term | AAACCTTAGCATCACAAAGAACAGTGAGCTCATTGGCCTTCTTGAA | 4894 |
| CGA-TGA | AAGACCATCCCTTCATTTTGAGTAAGTTACTTGTCTGTTCGTTGTGT | |
| | TCTCTATTCTCTTGATCTGGATCTTTCC | |
| | ACTCAAAATGAAGGGAT | 4895 |
| | ATCCCTTCATTTTGAGT | 4896 |
| Male-sterile | GGCTCGTGGTAAGATCCAGATCAAGAAAATAGAAAACCAAACAAAT | 4897 |
| DEF4 | AGGCAAGTGACTTAGTCAAAGAGAAGAAATGGGCTATTCAAGAAG | |
| Solanum tuberosum | GCTAATGAACTTACAGTTCTTTGTGATGCT | |
| Tyr21Term | AGCATCACAAAGAACTGTAAGTTCATTAGCCTTCTTGAATAGCCCA | 4898 |
| TAT-TAG | TTTCTTCTCTTTGACTAAGTCACTTGCCTATTTGTTTGGTTTTCTATT | |
| | TTCTTGATCTGGATCTTACCACGAGCC | |
| | GTGACTTAGTCAAAGAG | 4899 |
| | CTCTTTGACTAAGTCAC | 4900 |
| Male-sterile | CTCGTGGTAAGATCCAGATCAAGAAAATAGAAAACCAAACAAATAG | 4901 |
| DEF4 | GCAAGTGACTTATTGAAAGAGAAGAAATGGGCTATTCAAGAAGGC | |
| Solanum tuberosum | TAATGAACTTACAGTTCTTTGTGATGCTAA | |
| Ser22Term | TTAGCATCACAAAGAACTGTAAGTTCATTAGCCTTCTTGAATAGCC | 4902 |
| TCA-TGA | CATTTCTTCTCTTTCAATAAGTCACTTGCCTATTTGTTTGGTTTTCTA | |
| | TTTTCTTGATCTGGATCTTACCACGAG | |
| | GACTTATTGAAAGAGAA | 4903 |
| | TTCTCTTTCAATAAGTC | 4904 |
| Male-sterile | CGTGGTAAGATCCAGATCAAGAAAATAGAAAACCAAACAAATAGG | 4905 |
| DEF4 | CAAGTGACTTATTCATAGAGAAGAAATGGGCTATTCAAGAAGGCTA | |
| Solanum tuberosum | ATGAACTTACAGTTCTTTGTGATGCTAAAG | |
| Lys23Term | CTTTAGCATCACAAAGAACTGTAAGTTCATTAGCCTTCTTGAATAG | 4906 |
| AAG-TAG | CCCATTTCTTCTCTATGAATAAGTCACTTGCCTATTTGTTTGGTTTT | |
| | CTATTTTCTTGATCTGGATCTTACCACG | |
| | CTTATTCATAGAGAAGA | 4907 |
| | TCTTCTCTATGAATAAG | 4908 |
| Male-sterile | GGTAAGATCCAGATCAAGAAAATAGAAAACCAAACAAATAGGCAA | 4909 |
| DEF4 | GTGACTTATTCAAAGTGAAGAAATGGGCTATTCAAGAAGGCTAATG | |
| Solanum tuberosum | AACTTACAGTTCTTTGTGATGCTAAAGTTT | |
| Arg24Term | AAACTTTAGCATCACAAAGAACTGTAAGTTCATTAGCCTTCTTGAAT | 4910 |
| AGA-TGA | AGCCCATTTCTTCACTTTGAATAAGTCACTTGCCTATTTGTTTGGTT | |
| | TTCTATTTTCTTGATCTGGATCTTACC | |
| | ATTCAAAGTGAAGAAAT | 4911 |
| | ATTTCTTCACTTTGAAT | 4912 |
| Male-sterile | GCTAATGAACTTACTGTTCTTTGTGATGCTAAAGTTTCAATTGTTAT | 4913 |
| AP3 | GATTTCTAGTACTTGAAAACTTCATGAGTTTATAAGTCCCTCTATCA | |
| Lycopersicon | CGACCAAACAATTGTTCGATCTGTACC | |
| esculentum | GGTACAGATCGAACAATTGTTTGGTCGTGATAGAGGGACTTATAAA | 4914 |
| Gly21Term | CTCATGAAGTTTTCAAGTACTAGAAATCATAACAATTGAAACTTTAG | |
| GGA-TGA | CATCACAAAGAACAGTAAGTTCATTAGC | |
| | CTAGTACTTGAAAACTT | 4915 |
| | AAGTTTTCAAGTACTAG | 4916 |
| Male-sterile | AATGAACTTACTGTTCTTTGTGATGCTAAAGTTTCAATTGTTATGAT | 4917 |
| AP3 | TTCTAGTACTGGATAACTTCATGAGTTTATAAGTCCCTCTATCACGA | |
| Lycopersicon | CCAAACAATTGTTCGATCTGTACCAGA | |
| esculentum | TCTGGTACAGATCGAACAATTGTTTGGTCGTGATAGAGGGACTTAT | 4918 |
| Lys28Term | AAACTCATGAAGTTATCCAGTACTAGAAATCATAACAATTGAAACTT | |
| AAA-TAA | TAGCATCACAAAGAACAGTAAGTTCATT | |
| | GTACTGGATAACTTCAT | 4919 |
| | ATGAAGTTATCCAGTAC | 4920 |
| Male-sterile | ACTGTTCTTTGTGATGCTAAAGTTTCAATTGTTATGATTTCTAGTAC | 4921 |
| AP3 | TGGAAAACTTCATTAGTTTATAAGTCCCTCTATCACGACCAAACAAT | |
| Lycopersicon | TGTTCGATCTGTACCAGAAGACTATTG | |
| esculentum | CAATAGTCTTCTGGTACAGATCGAACAATTGTTTGGTCGTGATAGA | 4922 |
| Glu31Term | GGGACTTATAAACTAATGAAGTTTTCCAGTACTAGAAATCATAACA | |
| GAG-TAG | ATTGAAACTTTAGCATCACAAAGAACAGT | |
| | AACTTCATTAGTTTATA | 4923 |
| | TATAAACTAATGAAGTT | 4924 |
| Male-sterile | ATTGTTATGATTTCTAGTACTGGAAAACTTCATGAGTTTATAAGTCC | 4925 |
| AP3 | CTCTATCACGACCTAACAATTGTTCGATCTGTACCAGAAGACTATT | |
| Lycopersicon | GGAGTTGATATTTGGACTACTCACTATG | |
| esculentum | CATAGTGAGTAGTCCAAATATCAACTCCAATAGTCTTCTGGTACAG | 4926 |
| Lys40Term | ATCGAACAATTGTTAGGTCGTGATAGAGGGACTTATAAACTCATGA | |
| AAA-TAA | AGTTTTCCAGTACTAGAAATCATAACAAT | |
| | TCACGACCTAACAATTG | 4927 |
| | CAATTGTTAGGTCGTGA | 4928 |
| Male-sterile | GGGGCGGGGGAAGATTGAGATAAAGCGGATCGAGAACGCCACCA | 4929 |
| AP3 | ACAGGCAGGTGACCTAGTCCAAGCGCCGGTCGGGGATCATGAAG | |
| Triticum aestivum | AAGGCGCGGGAGCTCACCGTGCTCTGCGACGCC | |
| Tyr21Term | GGCGTCGCAGAGCACGGTGAGCTCCCGCGCCTTCTTCATGATCC | 4930 |
| TAC-TAG | CCGACCGGCGCTTGGACTAGGTCACCTGCCTGTTGGTGGCGTTCT | |
| | CGATCCGCTTTATCTCAATCTTCCCCCGCCCC | |
| | GTGACCTAGTCCAAGCG | 4931 |
| | CGCTTGGACTAGGTCAC | 4932 |
| Male-sterile | CGGGGGAAGATTGAGATAAAGCGGATCGAGAACGCCACCAACAG | 4933 |
| AP3 | GCAGGTGACCTACTCCTAGCGCCGGTCGGGGATCATGAAGAAGG | |
| Triticum aestivum | CGCGGGAGCTCACCGTGCTCTGCGACGCCCAGG | |
| Lys23Term | CCTGGGCGTCGCAGAGCACGGTGAGCTCCCGCGCCTTCTTCATG | 4934 |
| AAG-TAG | ATCCCCGACCGGCGCTAGGAGTAGGTCACCTGCCTGTTGGTGGC | |
| | GTTCTCGATCCGCTTTATCTCAATCTTCCCCCG | |
| | CCTACTCCTAGCGCCGG | 4935 |
| | CCGGCGCTAGGAGTAGG | 4936 |
| Male-sterile | TTGAGATAAAGCGGATCGAGAACGCCACCAACAGGCAGGTGACCT | 4937 |
| AP3 | ACTCCAAGCGCCGGTAGGGGATCATGAAGAAGGCGCGGGAGCTC | |
| Triticum aestivum | ACCGTGCTCTGCGACGCCCAGGTCGCCATCAT | |
| Ser26Term | ATGATGGCGACCTGGGCGTCGCAGAGCACGGTGAGCTCCCGCGC | 4938 |
| TCG-TAG | CTTCTTCATGATCCCCTACCGGCGCTTGGAGTAGGTCACCTGCCT | |
| | GTTGGTGGCGTTCTCGATCCGCTTTATCTCAA | |
| | GCGCCGGTAGGGGATCA | 4939 |
| | TGATCCCCTACCGGCGC | 4940 |
| Male-sterile | CGGATCGAGAACGCCACCAACAGGCAGGTGACCTACTCCAAGCG | 4941 |
| AP3 | CCGGTCGGGGATCATGTAGAAGGCGCGGGAGCTCACCGTGCTCT | |
| Triticum aestivum | GCGACGCCCAGGTCGCCATCATCATGTTCTCCT | |
| Lys30Term | AGGAGAACATGATGATGGCGACCTGGGCGTCGCAGAGCACGGTG | 4942 |
| AAG-TAG | AGCTCCCGCGCCTTCTACATGATCCCCGACCGGCGCTTGGAGTAG | |
| | GTCACCTGCCTGTTGGTGGCGTTCTCGATCCG | |
| | GGATCATGTAGAAGGCG | 4943 |
| | CGCCTTCTACATGATCC | 4944 |
| Male-sterile | GGGGCGCGGCAAGATCGAGATCAAGCGGATCGAGAACGCCACCA | 4945 |
| Silky1 | ACCGCCAGGTGACCTAGTCCAAGCGCCGGACGGGGATCATGAAG | |
| Zea mays | AAGGCACGCGAGCTCACCGTGCTCTGCGACGCC | |
| Tyr21Term | GGCGTCGCAGAGCACGGTGAGCTCGCGTGCCTTCTTCATGATCCC | 4946 |
| TAC-TAG | CGTCCGGCGCTTGGACTAGGTCACCTGGCGGTTGGTGGCGTTCT | |
| | CGATCCGCTTGATCTCGATCTTGCCGCGCCCC | |
| | GTGACCTAGTCCAAGCG | 4947 |
| | CGCTTGGACTAGGTCAC | 4948 |
| Male-sterile | CGCGGCAAGATCGAGATCAAGCGGATCGAGAACGCCACCAACCG | 4949 |
| Silky1 | CCAGGTGACCTACTCCTAGCGCCGGACGGGGATCATGAAGAAGG | |
| Zea mays | CACGCGAGCTCACCGTGCTCTGCGACGCCCAGG | |
| Lys23Term | CCTGGGCGTCGCAGAGCACGGTGAGCTCGCGTGCCTTCTTCATG | 4950 |
| AAG-TAG | ATCCCCGTCCGGCGCTAGGAGTAGGTCACCTGGCGGTTGGTGGC | |
| | GTTCTCGATCCGCTTGATCTCGATCTTGCCGCG | |
| | CCTACTCCTAGCGCCGG | 4951 |
| | CCGGCGCTAGGAGTAGG | 4952 |
| Male-sterile | CGGATCGAGAACGCCACCAACCGCCAGGTGACCTACTCCAAGCG | 4953 |
| Silky1 | CCGGACGGGGATCATGTAGAAGGCACGCGAGCTCACCGTGCTCT | |
| Zea mays | GCGACGCCCAGGTCGCCATCATCATGTTCTCCT | |
| Lys30Term | AGGAGAACATGATGATGGCGACCTGGGCGTCGCAGAGCACGGTG | 4954 |
| AAG-TAG | AGCTCGCGTGCCTTCTACATGATCCCCGTCCGGCGCTTGGAGTAG | |
| | GTCACCTGGCGGTTGGTGGCGTTCTCGATCCG | |
| | GGATCATGTAGAAGGCA | 4955 |
| | TGCCTTCTACATGATCC | 4956 |
| Male-sterile | ATCGAGAACGCCACCAACCGCGAGGTGACCTACTCCAAGCGCCG | 4957 |
| Silky1 | GACGGGGATCATGAAGTAGGCACGCGAGCTCACCGTGCTCTGCG | |
| Zea mays | ACGCCCAGGTCGCCATCATCATGTTCTCCTCCA | |
| Lys31Term | TGGAGGAGAACATGATGATGGCGACCTGGGCGTCGCAGAGCACG | 4958 |
| AAG-TAG | GTGAGCTCGCGTGCCTACTTCATGATCCCCGTCCGGCGCTTGGAG | |
| | TAGGTCACCTGGCGGTTGGTGGCGTTCTCGAT | |
| | TCATGAAGTAGGCACGC | 4959 |
| | GCGTGCCTACTTCATGA | 4960 |
| Male-sterile | GCTAGCTGCATTGTCCGGCGAGAGAGATAGCTGCTGCAGGGGGC | 4961 |
| AP3 | GGCCATGGGGAGGGGCTAGATCGAGATCAAGCGGATCGAGAACG | |
| Oryza sativa | CGACCAACAGGCAGGTGACCTACTCGAAGCGCC | |
| Lys5Term | GGCGCTTCGAGTAGGTCACCTGCCTGTTGGTCGCGTTCTCGATCC | 4962 |
| AAG-TAG | GCTTGATCTCGATCTAGCCCCTCCCCATGGCCGCCCCCTGCAGGA | |
| | GCTATCTCTCTCGCCGGACAATGCAGCTAGC | |
| | GGAGGGGCTAGATCGAG | 4963 |
| | CTCGATCTAGCCCCTCC | 4964 |
| Male-sterile | TGCATTGTCCGGCGAGAGAGATAGCTGCTGCAGGGGGCGGCCAT | 4965 |
| AP3 | GGGGAGGGGCAAGATCTAGATCAAGGGGATCGAGAACGCGACCA | |
| Oryza sativa | ACAGGCAGGTGACCTACTCGAAGCGCCGCACGG | |
| Glu7Term | CCGTGCGGCGCTTCGAGTAGGTCACCTGCCTGTTGGTCGCGTTCT | 4966 |
| GAG-TAG | CGATCCGCTTGATCTAGATCTTGCCCCTCCCCATGGCCGCCCCCT | |
| | GCAGCAGCTATCTCTCTCGCCGGACAATGCA | |
| | GCAAGATCTAGATCAAG | 4967 |
| | CTTGATCTAGATCTTGC | 4968 |
| Male-sterile | GTCCGGCGAGAGAGATAGCTGCTGCAGGGGGCGGCCATGGGGA | 4969 |
| AP3 | GGGGCAAGATCGAGATCTAGCGGATCGAGAACGCGACCAACAGG | |
| Oryza sativa | CAGGTGACCTACTCGAAGCGCCGCACGGGGATCA | |
| Lys9Term | TGATCCCCGTGCGGCGCTTCGAGTAGGTCACCTGCCTGTTGGTCG | 4970 |
| AAG-TAG | CGTTCTCGATCCGCTAGATCTCGATCTTGCCCCTCCCCATGGCCG | |
| | CCCCCTGCAGCAGCTATCTCTCTCGCCGGAC | |
| | TCGAGATCTAGCGGATC | 4971 |
| | GATCCGCTAGATCTCGA | 4972 |
| Male-sterile | GAGAGATAGCTGCTGCAGGGGGCGGCCATGGGGAGGGGCAAGA | 4973 |
| AP3 | TCGAGATCAAGCGGATCTAGAACGCGACCAACAGGCAGGTGACCT | |
| Oryza sativa | ACTCGAAGCGCCGCACGGGGATCATGAAGAAGG | |
| Glu12Term | CCTTCTTCATGATCCCCGTGCGGCGCTTCGAGTAGGTCACCTGCC | 4974 |
| GAG-TAG | TGTTGGTCGCGTTCTAGATCCGCTTGATCTCGATCTTGCCCCTCCC | |
| | CATGGCCGCCCCCTGCAGCAGCTATCTCTC | |
| | AGCGGATCTAGAACGCG | 4975 |
| | CGCGTTCTAGATCCGCT | 4976 |

TABLE 33

Oligonucleotides to produce male-sterile plants

| | |
|---|---|
| Phenotype, Gene, | SEQ |

| | | |
|---|---|---|
| Plant & Targeted | | ID |
| Alteration | Altering Oligos | NO: |

| | | |
|---|---|---|
| Male-sterile | TCTGTACTAATCAAATTTTGCCCTAAACGTTTTTGGCTTTGGAGCA | 4977 |
| AG | GCAATCACGGCGTAGCAATCGGAGCTAGGAGGAGATTCCTCTCC | |
| Arabidopsis thaliana | CTTGAGGAAATCTGGGAGAGGAAAGATCGAA | |
| Tyr35Term | TTCGATCTTTCCTCTCCCAGATTTCCTCAAGGGAGAGGAATCTCCT | 4978 |
| TAC-TAG | CCTAGCTCCGATTGCTACGCCGTGATTGCTGCTCCAAAGCCAAAA | |
| | ACGTTTAGGGCAAAATTTGATTAGTACAGA | |
| | ACGGCGTAGCAATCGGA | 4979 |
| | TCCGATTGCTACGCCGT | 4980 |
| Male-sterile | CTGTACTAATCAAATTTTGCCCTAAACGTTTTTGGCTTTGGAGCAG | 4981 |
| AG | CAATCACGGCGTACTAATCGGAGCTAGGAGGAGATTCCTCTCCCT | |
| Arabidopsis thaliana | TGAGGAAATCTGGGAGAGGAAAGATCGAAA | |
| Gln36Term | TTTCGATCTTTCCTCTCCCAGATTTCCTCAAGGGAGAGGAATCTCC | 4982 |
| CAA-TAA | TCCTAGCTCCGATTAGTACGCCGTGATTGCTGCTCCAAAGCCAAA | |
| | AACGTTTAGGGCAAAATTTGATTAGTACAG | |
| | CGGCGTACTAATCGGAG | 4983 |
| | CTCCGATTAGTACGCCG | 4984 |
| Male-sterile | ACTAATCAAATTTTGCCCTAAACGTTTTTGGCTTTGGAGCAGCAAT | 4985 |
| AG | CACGGCGTACCAATAGGAGCTAGGAGGAGATTCCTCTCCCTTGA | |
| Arabidopsis thaliana | GGAAATCTGGGAGAGGAAAGATCGAAATCAA | |
| Ser31Term | TTGATTTCGATCTTTCCTCTCCCAGATTTCCTCAAGGGAGAGGAAT | 4986 |
| TCG-TAG | CTCCTCCTAGCTCCTATTGGTACGCCGTGATTGCTGCTCCAAAGC | |
| | CAAAAACGTTTAGGGCAAAATTTGATTAGT | |
| | GTACCAATAGGAGCTAG | 4987 |
| | CTAGCTCCTATTGGTAC | 4988 |
| Male-sterile | TAATCAAATTTTGCCCTAAACGTTTTTGGCTTTGGAGCAGCAATCA | 4989 |
| AG | CGGCGTACCAATCGTAGCTAGGAGGAGATTCCTCTCCCTTGAGGA | |
| Arabidopsis thaliana | AATCTGGGAGAGGAAAGATCGAAATCAAAC | |
| Glu38Term | GTTTGATTTCGATCTTTCCTCTCCCAGATTTCCTCAAGGGAGAGGA | 4990 |
| GAG-TAG | ATCTCCTCCTAGCTACGATTGGTACGCCGTGATTGCTGCTCCAAA | |
| | GCCAAAAACGTTTAGGGCAAAATTTGATTA | |
| | ACCAATCGTAGCTAGGA | 4991 |
| | TCCTAGCTACGATTGGT | 4992 |
| Male-sterile | CTCTCCCACTTCTTTTCGGTGGTTTATTCATTTGGTGACGATATCA | 4993 |
| AG | CAGAAGCAATGGATTAAGGTGGGAGTAGTCACGATGCAGAGAGT | |
| Brassica napus | AGCAAGAAGATAGGTAGAGGGAAGATAGAGA | |
| Glu3Term | TCTCTATCTTCCCTCTACCTATCTTCTTGCTACTCTCTGCATCGTGA | 4994 |
| GAA-TAA | CTACTCCCACCTTAATCCATTGCTTCTGTGATATCGTCACCAAATG | |
| | AATAAACCACCGAAAAGAAGTGGGAGAG | |
| | CAATGGATTAAGGTGGG | 4995 |
| | CCCACCTTAATCCATTG | 4996 |
| Male-sterile | TATTCATTTGGTGACGATATCACAGAAGCAATGGATGAAGGTGGG | 4997 |
| AG | AGTAGTCACGATGCATAGAGTAGCAAGAAGATAGGTAGAGGGAA | |
| Brassica napus | GATAGAGATAAAGAGGATAGAGAACACAACAA | |
| Glu11Term | TTGTTGTGTTCTCTATCCTCTTTATCTCTATCTTCCCTCTACCTATC | 4998 |
| GAG-TAG | TTCTTGCTACTCTATGCATCGTGACTACTCCCACCTTCATCCATTG | |
| | CTTCTGTGATATCGTCACCAAATGAATA | |
| | ACGATGCATAGAGTAGC | 4999 |
| | GCTACTCTATGCATCGT | 5000 |
| Male-sterile | GGTGACGATATCACAGAAGCAATGGATGAAGGTGGGAGTAGTCA | 5001 |
| AG | CGATGCAGAGAGTAGCTAGAAGATAGGTAGAGGGAAGATAGAGA | |
| Brassica napus | TAAAGAGGATAGAGAACACAACAAATCGTCAAG | |
| Lys14Term | CTTGACGATTTGTTGTGTTCTCTATCCTCTTTATCTCTATCTTCCCT | 5002 |
| AAG-TAG | CTACCTATCTTCTAGCTACTCTCTGCATCGTGACTACTCCCACCTT | |
| | CATCCATTGCTTCTGTGATATCGTCACC | |
| | AGAGTAGCTAGAAGATA | 5003 |
| | TATCTTCTAGCTACTCT | 5004 |
| Male-sterile | GACGATATCACAGAAGCAATGGATGAAGGTGGGAGTAGTCACGA | 5005 |
| AG | TGCAGAGAGTAGCAAGTAGATAGGTAGAGGGAAGATAGAGATAAA | |
| Brassica napus | GAGGATAGAGAACACAACAAATCGTCAAGTAA | |
| Lys15Term | TTACTTGACGATTTGTTGTGTTCTCTATCCTCTTTATCTCTATCTTC | 5006 |
| AAG-TAG | CCTCTACCTATCTACTTGCTACTCTCTGCATCGTGACTACTCCCAC | |
| | CTTCATCCATTGCTTCTGTGATATCGTC | |
| | GTAGCAAGTAGATAGGT | 5007 |
| | ACCTATCTACTTGCTAC | 5008 |
| Male-sterile | CAACCAAAAAACTTAAAAATCTTCTCTTTCCTTTCCTTACAAGGTGA | 5009 |
| AG | AGTAATGGACTTCTAAAGTGATCTAACCAGAGAGATCTCACCACAA | |
| Lycopersicon | AGGAAACTAGGAAGGGGGAAAATTGAGA | |
| esculentum | TCTCAATTTTCCCCCTTCCTAGTTTCCTTTGTGGTGAGATCTCTCT | 5010 |
| Glu4Term | GGTTAGATCACTTTAGAAGTCCATTACTTCACCTTGTAAGGAAAGG | |
| CAA-TAA | AAAGAGAAGATTTTTAAGTTTTTTGGTTG | |
| | TGGACTTCTAAAGTGAT | 5011 |
| | ATCACTTTAGAAGTCCA | 5012 |
| Male-sterile | AAAATCTTCTCTTTCCTTTCCTTACAAGGTGAAGTAATGGACTTCC | 5013 |
| AG | AAAGTGATCTAACCTGAGAGATCTCACCACAAAGGAAACTAGGAA | |
| Lycopersicon | GGGGGAAAATTGAGATCAAAAGGATCGAAA | |
| esculentum | TTTCGATCCTTTTGATCTCAATTTTCCCCCTTCCTAGTTTCCTTTGT | 5014 |
| Arg9Term | GGTGAGATCTCTCAGGTTAGATCACTTTGGAAGTCCATTACTTCAC | |
| AGA-TGA | CTTGTAAGGAAAGGAAAGAGAAGATTTT | |
| | ATCTAACCTGAGAGATC | 5015 |
| | GATCTCTCAGGTTAGAT | 5016 |
| Male-sterile | ATCTTCTCTTTCCTTTCCTTACAAGGTGAAGTAATGGACTTCCAAA | 5017 |
| AG | GTGATCTAACCAGATAGATCTCACCACAAAGGAAACTAGGAAGGG | |
| Lycopersicon | GGAAAATTGAGATCAAAAGGATCGAAAACA | |
| esculentum | TGTTTTCGATCCTTTTGATCTCAATTTTCCCCCTTCCTAGTTTCCTT | 5018 |
| Glu10Term | TGTGGTGAGATCTATCTGGTTAGATCACTTTGGAAGTCCATTACTT | |
| GAG-TAG | CACCTTGTAAGGAAAGGAAAGAGAAGAT | |
| | TAACCAGATAGATCTCA | 5019 |
| | TGAGATCTATCTGGTTA | 5020 |
| Male-sterile | CTTTCCTTTCCTTACAAGGTGAAGTAATGGACTTCCAAAGTGATCT | 5021 |
| AG | AACCAGAGAGATCTGACCACAAAGGAAACTAGGAAGGGGGAAAA | |
| Lycopersicon | TTGAGATCAAAAGGATCGAAAACACGACGAA | |
| esculentum | TTCGTCGTGTTTTCGATCCTTTTGATCTCAATTTTCCCCCTTCCTAG | 5022 |
| Ser12Term | TTTCCTTTGTGGTCAGATCTCTCTGGTTAGATCACTTTGGAAGTCC | |
| TCA-TGA | ATTACTTCACCTTGTAAGGAAAGGAAAG | |
| | AGAGATCTGACCACAAA | 5023 |
| | TTTGTGGTCAGATCTCT | 5024 |
| Male-sterile | GTACTCTCTATTTTCATCTTCCAACCCTTTCTTTCCTTACCAGGTGA | 5025 |
| NAG1 | AAGTATGGACTTCTAAAGTGATCTAACAAGAGAGATCTCTCCACAA | |
| Nicotiana tabacum | AGGAAACTGGGAAGAGGAAAGATTGAGA | |
| Gln4Term | TCTCAATCTTTCCTCTTCCCAGTTTCCTTTGTGGAGAGATCTCTCTT | 5026 |
| CAA-TAA | GTTAGATCACTTTAGAAGTCCATACTTTCACCTGGTAAGGAAAGAA | |
| | AGGGTTGGAAGATGAAAATAGAGAGTAC | |
| | TGGACTTCTAAAGTGAT | 5027 |
| | ATCACTTTAGAAGTCCA | 5028 |
| Male-sterile | ATCTTCCAACCCTTTCTTTCCTTACCAGGTGAAAGTATGGACTTCC | 5029 |
| NAG1 | AAAGTGATCTAACATGAGAGATCTCTCCACAAAGGAAACTGGGAA | |
| Nicotiana tabacum | GAGGAAAGATTGAGATCAAACGGATCGAAA | |
| Arg9Term | TTTCGATCCGTTTGATCTCAATCTTTCCTCTTCCCAGTTTCCTTTGT | 5030 |
| AGA-TGA | GGAGAGATCTCTCATGTTAGATCACTTTGGAAGTCCATACTTTCAC | |
| | CTGGTAAGGAAAGAAAGGGTTGGAAGAT | |
| | ATCTAACATGAGAGATC | 5031 |
| | GATCTCTCATGTTAGAT | 5032 |
| Male-sterile | TTCCAACCCTTTCTTTCCTTACCAGGTGAAAGTATGGACTTCCAAA | 5033 |
| NAG1 | GTGATCTAACAAGATAGATCTCTCCACAAAGGAAACTGGGAAGAG | |
| Nicotiana tabacum | GAAAGATTGAGATCAAACGGATCGAAAACA | |
| Glu10Term | TGTTTTCGATCCGTTTGATCTCAATCTTTCCTCTTCCCAGTTTCCTT | 5034 |
| GAG-TAG | TGTGGAGAGATCTATCTTGTTAGATCACTTTGGAAGTCCATACTTT | |
| | CACCTGGTAAGGAAAGAAAGGGTTGGAA | |
| | TAACAAGATAGATCTCT | 5035 |
| | AGAGATCTATCTTGTTA | 5036 |
| Male-sterile | CTTTCCTTACCAGGTGAAAGTATGGACTTCCAAAGTGATCTAACAA | 5037 |
| NAG1 | GAGAGATCTCTCCATAAAGGAAACTGGGAAGAGGAAAGATTGAGA | |
| Nicotiana tabacum | TCAAACGGATCGAAAACACAACGAATCGTC | |
| Gln14Term | GACGATTCGTTGTGTTTTCGATCCGTTTGATCTCAATCTTTCCTCTT | 5038 |
| CAA-TAA | CCCAGTTTCCTTTATGGAGAGATCTCTCTTGTTAGATCACTTTGGA | |
| | AGTCCATACTTTCACCTGGTAAGGAAAG | |
| | TCTCTCCATAAAGGAAA | 5039 |
| | TTTCCTTTATGGAGAGA | 5040 |
| Male-sterile | GCCTATGAAAACAAACCCAACACGGTCCTGGACGCTGATGCCCAA | 5041 |
| AG | AGAAGATTGGGAAGGTGAAAGATCGAGATCAAGCGGATCGAAAA | |
| Rosa hybrida | CACCACCAATCGTCAAGTCACCTTCTGCAAAA | |
| Gly22Term | TTTTGCAGAAGGTGACTTGACGATTGGTGGTGTTTTCGATCCGCT | 5042 |
| GGA-TGA | TGATCTCGATCTTTCACCTTCCCAATCTTCTTTGGGCATCAGCGTC | |
| | CAGGACCGTGTTGGGTTTGTTTTCATAGGC | |
| | TGGGAAGGTGAAAGATC | 5043 |
| | GATCTTTCACCTTCCCA | 5044 |
| Male-sterile | TATGAAAACAAACCCAACACGGTCCTGGACGCTGATGCCCAAAGA | 5045 |
| AG | AGATTGGGAAGGGGATAGATCGAGATCAAGCGGATCGAAAACAC | |
| Rosa hybrida | CACCAATCGTCAAGTCACCTTCTGCAAAAGGC | |
| Lys23Term | GCCTTTTGCAGAAGGTGACTTGACGATTGGTGGTGTTTTCGATCC | 5046 |
| AAG-TAG | GCTTGATCTCGATCTATCCCCTTCCCAATCTTCTTTGGGCATCAGC | |
| | GTCCAGGACCGTGTTGGGTTTGTTTTCATA | |
| | GAAGGGGATAGATCGAG | 5047 |
| | CTCGATCTATCCCCTTC | 5048 |
| Male-sterile | AACAAACCCAACACGGTCCTGGACGCTGATGCCCAAAGAAGATTG | 5049 |
| AG | GGAAGGGGAAAGATCTAGATCAAGCGGATCGAAAACACCACCAA | |
| Rosa hybrida | TCGTCAAGTCACCTTCTGCAAAAGGCGCAATG | |
| Glu25Term | CATTGCGCCTTTTGCAGAAGGTGACTTGACGATTGGTGGTGTTTT | 5050 |
| GAG-TAG | CGATCCGCTTGATCTAGATCTTTCCCCTTCCCAATCTTCTTTGGGC | |
| | ATCAGCGTCCAGGACCGTGTTGGGTTTGTT | |
| | GAAAGATCTAGATCAAG | 5051 |
| | CTTGATCTAGATCTTTC | 5052 |
| Male-sterile | CCCAACACGGTCCTGGACGCTGATGCCCAAAGAAGATTGGGAAG | 5053 |
| AG | GGGAAAGATCGAGATCTAGCGGATCGAAAACACCACCAATCGTCA | |
| Rosa hybrida | AGTCACCTTCTGCAAAAGGCGCAATGGTTTGC | |
| Lys27 | GCAAACCATTGCGCCTTTTGCAGAAGGTGACTTGACGATTGGTGG | 5054 |
| AAG-TAG | TGTTTTCGATCCGCTAGATCTCGATCTTTCCCCTTCCCAATCTTCT | |
| | TTGGGCATCAGCGTCCAGGACCGTGTTGGG | |
| | TCGAGATCTAGCGGATC | 5055 |
| | GATCCGCTAGATCTCGA | 5056 |
| Male-sterile | CAATTGCCTGTTTTTATTTTTTTTCTTTTTGACTAAGTAGAAATGGC | 5057 |
| far | GTCTCTAAGCGATTAATCGACCGAGGTATCGCCCGAGAGGAAAAT | |
| Antirrhinum majus | CGGGAGAGGAAAGATCGAGATCAAACGGA | |
| Gln7Term | TCCGTTTGATCTCGATCTTTCCTCTCCCGATTTTCCTCTCGGGCGA | 5058 |
| CAA-TAA | TACCTCGGTCGATTAATCGCTTAGAGACGCCATTTCTACTTAGTCA | |
| | AAAAGAAAAAAAATAAAAACAGGCAATTG | |
| | TAAGCGATTAATCGACC | 5059 |
| | GGTCGATTAATCGCTTA | 5060 |
| Male-sterile | GTTTTTATTTTTTTTCTTTTTGACTAAGTAGAAATGGCGTCTCTAAG | 5061 |
| far | CGATCAATCGACCTAGGTATCGCCCGAGAGGAAAATCGGGAGAG | |
| Antirrhinum majus | GAAAGATCGAGATCAAACGGATCGAAAACA | |
| Glu10Term | TGTTTTCGATCCGTTTGATCTCGATCTTTCCTCTCCCGATTTTCCTC | 5062 |
| GAG-TAG | TCGGGCGATACCTAGGTCGATTGATCGCTTAGAGACGCCATTTCT | |
| | ACTTAGTCAAAAAGAAAAAAAATAAAAAC | |
| | AATCGACCTAGGTATCG | 5063 |
| | CGATACCTAGGTCGATT | 5064 |
| Male-sterile | TTTCTTTTTGACTAAGTAGAAATGGCGTCTCTAAGCGATCAATCGA | 5065 |
| far | CCGAGGTATCGCCCTAGAGGAAAATCGGGAGAGGAAAGATCGAG | |
| Antirrhinum majus | ATCAAACGGATCGAAAACAAAACAAATCAAC | |
| Glu14Term | GTTGATTTGTTTTGTTTTCGATCCGTTTGATCTCGATCTTTCCTCTC | 5066 |
| GAG-TAG | CCGATTTTCCTCTAGGGCGATACCTCGGTCGATTGATCGCTTAGA | |
| | GACGCCATTTCTACTTAGTCAAAAAGAAA | |
| | TATCGCCCTAGAGGAAA | 5067 |
| | TTTCCTCTAGGGCGATA | 5068 |
| Male-sterile | TTTGACTAAGTAGAAATGGCGTCTCTAAGCGATCAATCGACCGAG | 5069 |
| far | GTATCGCCCGAGAGGTAAATCGGGAGAGGAAAGATCGAGATCAA | |
| Antirrhinum majus | ACGGATCGAAAACAAAACAAATCAACAGGTTA | |
| Lys16Term | TAACCTGTTGATTTGTTTTGTTTTCGATCCGTTTGATCTCGATCTTT | 5070 |
| AAA-TAA | CCTCTCCCGATTTACCTCTCGGGCGATACCTCGGTCGATTGATCG | |
| | CTTAGAGACGCCATTTCTACTTAGTCAAA | |
| | CCGAGAGGTAAATCGGG | 5071 |
| | CCCGATTTACCTCTCGG | 5072 |
| Male-sterile | TGTCCAAGCATTATCAGTCACCACTCACAAGAATGATTAAGGAAGA | 5073 |
| AG | AGGAAAGGGTAAGTAGCAAATAAAGGGGATGTTCCAGAATCAAGA | |
| Cucumis sativus | AGAGAAGATGTCAGACTCGCCTCAGAGGAA | |
| Leu21Term | TTCCTCTGAGGCGAGTCTGACATCTTCTCTTCTTGATTCTGGAACA | 5074 |
| TTG-TAG | TCCCCTTTATTTGCTACTTACCCTTTCCTTCTTCCTTAATCATTCTT | |
| | GTGAGTGGTGACTGATAATGCTTGGACA | |
| | GGGTAAGTAGCAAATAA | 5075 |
| | TTATTTGCTACTTACCC | 5076 |
| Male-sterile | TCCAAGCATTATCAGTCACCACTCACAAGAATGATTAAGGAAGAA | 5077 |
| AG | GGAAAGGGTAAGTTGTAAATAAAGGGGATGTTCCAGAATCAAGAA | |
| Cucumis sativus | GAGAAGATGTCAGACTCGCCTCAGAGGAAGA | |
| Gln22Term | TCTTCCTCTGAGGCGAGTCTGACATCTTCTCTTCTTGATTCTGGAA | 5078 |
| CAA-TAA | CATCCCCTTTATTTACAACTTACCCTTTCCTTCTTCCTTAATCATTC | |
| | TTGTGAGTGGTGACTGATAATGCTTGGA | |
| | GTAAGTTGTAAATAAAG | 5079 |
| | CTTTATTTACAACTTAC | 5080 |
| Male-sterile | CATTATCAGTCACCACTCACAAGAATGATTAAGGAAGAAGGAAAG | 5081 |
| AG | GGTAAGTTGCAAATATAGGGGATGTTCCAGAATCAAGAAGAGAAG | |
| Cucumis sativus | ATGTCAGACTCGCCTCAGAGGAAGATGGGAA | |
| Lys24Term | TTCCCATCTTCCTCTGAGGCGAGTCTGACATCTTCTCTTCTTGATT | 5082 |
| AAG-TAG | CTGGAACATCCCCTATATTTGCAACTTACCCTTTCCTTCTTCCTTAA | |
| | TCATTCTTGTGAGTGGTGACTGATAATG | |
| | TGCAAATATAGGGGATG | 5083 |
| | CATCCCCTATATTTGCA | 5084 |
| Male-sterile | CCACTCACAAGAATGATTAAGGAAGAAGGAAAGGGTAAGTTGCAA | 5085 |
| AG | ATAAAGGGGATGTTCTAGAATCAAGAAGAGAAGATGTCAGACTCG | |
| Cucumis sativus | CCTCAGAGGAAGATGGGAAGAGGAAAGATTG | |
| Gln28Term | CAATCTTTCCTCTTCCCATCTTCCTCTGAGGCGAGTCTGACATCTT | 5086 |
| CAG-TAG | CTCTTCTTGATTCTAGAACATCCCCTTTATTTGCAACTTACCCTTTC | |
| | CTTCTTCCTTAATCATTCTTGTGAGTGG | |
| | GGATGTTCTAGAATCAA | 5087 |
| | TTGATTCTAGAACATCC | 5088 |
| Male-sterile | CCACCACCACCACCACCACCACCACCACACCATGCTCAACATGAT | 5089 |
| AG | GACTGATCTGAGCTGAGGGCCGTCGTCCAAGGTCAAGGAGCAGG | |
| Zea mays | TGGCGGCGGCGCCGACGGGCTCCGGCGACAGG | |
| Cys10Term | CCTGTCGCCGGAGCCCGTCGGCGCCGCCGCCACCTGCTCCTTGA | 5090 |
| TGC-TGA | CCTTGGACGACGGCCCTCAGCTCAGATCAGTCATCATGTTGAGCA | |
| | TGGTGTGGTGGTGGTGGTGGTGGTGGTGGTGG | |
| | CTGAGCTGAGGGCCGTC | 5091 |
| | GACGGCCCTCAGCTCAG | 5092 |
| Male-sterile | ACCACCACCACCACCACCACACCATGCTCAACATGATGACTGATC | 5093 |
| AG | TGAGCTGCGGGCCGTAGTCCAAGGTCAAGGAGCAGGTGGCGGC | |
| Zea mays | GGCGCCGACGGGCTCCGGCGACAGGCAGGGGCA | |
| Ser13Term | TGCCCCTGCCTGTCGCCGGAGCCCGTCGGCGCCGCCGCCACCT | 5094 |
| TCG-TAG | GCTCCTTGACCTTGGACTACGGCCCGCAGCTCAGATCAGTCATCA | |
| | TGTTGAGCATGGTGTGGTGGTGGTGGTGGTGGT | |
| | CGGGCCGTAGTCCAAGG | 5095 |
| | CCTTGGACTACGGCCCG | 5096 |
| Male-sterile | CACCACCACCACCACACCATGCTCAACATGATGACTGATCTGAGC | 5097 |
| AG | TGCGGGCCGTCGTCCTAGGTCAAGGAGCAGGTGGCGGCGGCGC | |
| Zea mays | CGACGGGCTCCGGCGACAGGCAGGGGCAGGGGA | |
| Lys15Term | TCCCCTGCCCCTGCCTGTCGCCGGAGCCCGTCGGCGCCGCCGC | 5098 |
| AAG-TAG | CACCTGCTCCTTGACCTAGGACGACGGCCCGCAGCTCAGATCAG | |
| | TCATCATGTTGAGCATGGTGTGGTGGTGGTGGTG | |
| | CGTCGTCCTAGGTCAAG | 5099 |
| | CTTGACCTAGGACGACG | 5100 |
| Male-sterile | CACCACCACACCATGCTCAACATGATGACTGATCTGAGCTGCGGG | 5101 |
| AG | CCGTCGTCCAAGGTCTAGGAGCAGGTGGCGGCGGCGCCGACGG | |
| Zea mays | GCTCCGGCGACAGGCAGGGGCAGGGGAGAGGCA | |
| Lys17Term | TGCCTCTCCCCTGCCCCTGCCTGTCGCCGGAGCCCGTCGGCGCC | 5102 |
| AAG-TAG | GCCGCCACCTGCTCCTAGACCTTGGACGACGGCCCGCAGCTCAG | |
| | ATCAGTCATCATGTTGAGCATGGTGTGGTGGTG | |
| | CCAAGGTCTAGGAGCAG | 5103 |
| | CTGCTCCTAGACCTTGG | 5104 |
| Male-sterile | TCCTACCTTTTCTCCTTCAGACCTCAAAATCTGTGTGATAGGAACA | 5105 |
| AG | AGAGCATGCACATCTGAGAAGAGGAGGCTACACCATCCACAGTAA | |
| Zea mays | CAGGCATCATGTCGACCCTGACTTCGGCGG | |
| Arg4Term | CCGCCGAAGTCAGGGTCGACATGATGCCTGTTACTGTGGATGGT | 5106 |
| CGA-TGA | GTAGCCTCCTCTTCTCAGATGTGCATGCTCTTGTTCCTATCACACA | |
| | GATTTTGAGGTCTGAAGGAGAAAAGGTAGGA | |
| | TGCACATCTGAGAAGAG | 5107 |
| | CTCTTCTCAGATGTGCA | 5108 |
| Male-sterile | TACCTTTTCTCCTTCAGACCTCAAAATCTGTGTGATAGGAACAAGA | 5109 |
| AG | GCATGCACATCCGATAAGAGGAGGCTACACCATCCACAGTAACAG | |
| Zea mays | GCATCATGTCGACCCTGACTTCGGCGGGGC | |
| Glu5Term | GCCCCGCCGAAGTCAGGGTCGACATGATGCCTGTTACTGTGGAT | 5110 |
| GAA-TAA | GGTGTAGCCTCCTCTTATCGGATGTGCATGCTCTTGTTCCTATCAC | |
| | ACAGATTTTGAGGTCTGAAGGAGAAAAGGTA | |
| | ACATCCGATAAGAGGAG | 5111 |
| | CTCCTCTTATCGGATGT | 5112 |
| Male-sterile | CTTTTCTCCTTCAGACCTCAAAATCTGTGTGATAGGAACAAGAGCA | 5113 |
| AG | TGCACATCCGAGAATAGGAGGCTACACCATCCACAGTAACAGGCA | |
| Zea mays | TCATGTCGACCCTGACTTCGGCGGGGCAGC | |
| Glu6Term | GCTGCCCCGCCGAAGTCAGGGTCGACATGATGCCTGTTACTGTG | 5114 |
| GAG-TAG | GATGGTGTAGCCTCCTATTCTCGGATGTGCATGCTCTTGTTCCTAT | |
| | CACACAGATTTTGAGGTCTGAAGGAGAAAAG | |
| | TCCGAGAATAGGAGGCT | 5115 |
| | AGCCTCCTATTCTCGGA | 5116 |
| Male-sterile | TTCTCCTTCAGACCTCAAAATCTGTGTGATAGGAACAAGAGCATG | 5117 |
| AG | CACATCCGAGAAGAGTAGGCTACACCATCCACAGTAACAGGCATC | |
| Zea mays | ATGTCGACCCTGACTTCGGCGGGGCAGCAGA | |
| Glu7Term | TCTGCTGCCCCGCCGAAGTCAGGGTCGACATGATGCCTGTTACT | 5118 |
| GAG-TAG | GTGGATGGTGTAGCCTACTCTTCTCGGATGTGCATGCTCTTGTTC | |
| | CTATCACACAGATTTTGAGGTCTGAAGGAGAA | |
| | GAGAAGAGTAGGCTACA | 5119 |
| | TGTAGCCTACTCTTCTC | 5120 |
| Male-sterile | GCTGGGTCAGGATCGTCGGCGGCGGTGGCGGCGGGGAGCAGC | 5121 |
| AG | GAGAAGATGGGGAGGGGGTAGATCGAGATAAAGCGGATCGAGAA | |
| Oryza sativa | CACGACGAACCGGCAGGTGACCTTCTGCAAGCGCC | |
| Lys5Term | GGCGCTTGCAGAAGGTCACCTGCCGGTTCGTCGTGTTCTCGATC | 5122 |
| AAG-TAG | CGCTTTATCTCGATCTACCCCCTCCCCATCTTCTCGCTGCTCCCC | |
| | GCCGCCACCGCCGCCGACGATCCTGACCCAGC | |
| | GGAGGGGGTAGATCGAG | 5123 |
| | CTCGATCTACCCCCTCC | 5124 |
| Male-sterile | TCAGGATCGTCGGCGGCGGTGGCGGCGGGGAGCAGCGAGAAGA | 5125 |
| AG | TGGGGAGGGGGAAGATCTAGATAAAGCGGATCGAGAACACGACG | |
| Oryza sativa | AACCGGCAGGTGACCTTCTGCAAGCGCCGCAATG | |
| Glu7Term | CATTGCGGCGCTTGCAGAAGGTCACCTGCCGGTTCGTCGTGTTCT | 5126 |
| GAG-TAG | CGATCCGCTTTATCTAGATCTTCCCCCTCCCCATCTTCTCGCTGCT | |
| | CCCCGCCGCCACCGCCGCCGACGATCCTGA | |
| | GGAAGATCTAGATAAAG | 5127 |
| | CTTTATCTAGATCTTCC | 5128 |
| Male-sterile | TCGTCGGCGGCGGTGGCGGCGGGGAGCAGCGAGAAGATGGGG | 5129 |
| AG | AGGGGGAAGATCGAGATATAGCGGATCGAGAACACGACGAACCG | |
| Oryza sativa | GCAGGTGACCTTCTGCAAGCGCCGCAATGGCCTCC | |
| Lys9Term | GGAGGCCATTGCGGCGCTTGCAGAAGGTCACCTGCCGGTTCGTC | 5130 |
| AAG-TAG | GTGTTCTCGATCCGCTATATCTCGATCTTCCCCCTCCCCATCTTCT | |
| | CGCTGCTCCCCGCCGCCACCGCCGCCGACGA | |
| | TCGAGATATAGCGGATC | 5131 |
| | GATCCGCTATATCTCGA | 5132 |
| Male-sterile | GCGGTGGCGGCGGGGAGCAGCGAGAAGATGGGGAGGGGGAAG | 5133 |
| AG | ATCGAGATAAAGCGGATCTAGAACACGACGAACCGGCAGGTGAC | |
| Oryza sativa | CTTCTGCAAGCGCCGCAATGGCCTCCTGAAGAAGG | |
| Glu12Term | CCTTCTTCAGGAGGCCATTGCGGCGCTTGCAGAAGGTCACCTGC | 5134 |
| GAG-TAG | CGGTTCGTCGTGTTCTAGATCCGCTTTATCTCGATCTTCCCCCTCC | |
| | CCATCTTCTCGCTGCTCCCCGCCGCCACCGC | |
| | AGCGGATCTAGAACACG | 5135 |
| | CGTGTTCTAGATCCGCT | 5136 |

TABLE 34
Oligonucleotides to produce male-sterile plants

| Phenotype, Gene, | | |
|---|---|---|
| Plant & Targeted | | SEQ ID |
| Alteration | Altering Oligos | NO: |
| Male-sterile | GGGAAGAGGGAAAATAGAAATAAAAAGAATAGAGAACTCAAGCAA | 5137 |
| PI | TAGACAAGTTACATAGTCAAAGAGAAGAAATGGTATCATCAAAAAA | |
| Cucumis sativus | GCCAAAGAAATTACTGTTCTTTGCGATGCT | |
| Tyr21 Term | AGCATCGCAAAGAACAGTAATTTCTTTGGCTTTTTTGATGATACCAT | 5138 |
| TAT-TAG | TTCTTCTCTTTGACTATGTAACTTGTCTATTGCTTGAGTTCTCTATTC | |
| | TTTTTATTTCTATTTTCCCTCTTCCC | |
| | GTTACATAGTCAAAGAG | 5139 |
| | CTCTTTGACTATGTAAC | 5140 |
| Male-sterile | GAAGAGGGAAAATAGAAATAAAAAGAATAGAGAACTCAAGCAATA | 5141 |
| PI | GACAAGTTACATATTGAAAGAGAAGAAATGGTATCATCAAAAAAGC | |
| Cucumis sativus | CAAAGAAATTACTGTTCTTTGCGATGCTCA | |
| Ser22Term | TGAGCATCGCAAAGAACAGTAATTTCTTTGGCTTTTTTGATGATAC | 5142 |
| TCA-TGA | CATTTCTTCTCTTTCAATATGTAACTTGTCTATTGCTTGAGTTCTCTA | |
| | TTCTTTTTATTTCTATTTTCCCTCTTC | |
| | TACATATTGAAAGAGAA | 5143 |
| | TTCTCTTTCAATATGTA | 5144 |
| Male-sterile | AGAGGGAAAATAGAAATAAAAAGAATAGAGAACTCAAGCAATAGAC | 5145 |
| PI | AAGTTACATATTCATAGAGAAGAAATGGTATCATCAAAAAAGCCAA | |
| Cucumis sativus | AGAAATTACTGTTCTTTGCGATGCTCAAG | |
| Lys23Term | CTTGAGCATCGCAAAGAACAGTAATTTCTTTGGCTTTTTTGATGATA | 5146 |
| AAG-TAG | CCATTTCTTCTCTATGAATATGTAACTTGTCTATTGCTTGAGTTCTC | |
| | TATTCTTTTTATTTCTATTTTCCCTCT | |
| | CATATTCATAGAGAAGA | 5147 |
| | TCTTCTCTATGAATATG | 5148 |
| Male-sterile | GGGAAAATAGAAATAAAAAGAATAGAGAACTCAAGCAATAGACAAG | 5149 |
| PI | TTACATATTCAAAGTGAAGAAATGGTATCATCAAAAAAGCCAAAGA | |
| Cucumis sativus | AATTACTGTTCTTTGCGATGCTCAAGTTT | |
| Arg24Term | AAACTTGAGCATCGCAAAGAACAGTAATTTCTTTGGCTTTTTTGATG | 5150 |
| AGA-TGA | ATACCATTTCTTCACTTTGAATATGTAACTTGTCTATTGCTTGAGTT | |
| | CTCTATTCTTTTTATTTCTATTTTCCC | |
| | ATTCAAAGTGAAGAAAT | 5151 |
| | ATTTCTTCACTTTGAAT | 5152 |
| Male-sterile | GGGACGTGGGAAGGTTGAGATCAAGAGGATTGAGAACTCAAGTAA | 5153 |
| PI | CAGGCAGGTGACCTAGTCCAAGAGGAGGAATGGGATTATCAAGAA | |
| Malus domestica | GGCAAAGGAGATCACTGTTCTATGTGATGCT | |
| Tyr21Term | AGCATCACATAGAACAGTGATCTCCTTTGCCTTCTTGATAATCCCA | 5154 |
| TAG-TAG | TTCCTCCTCTTGGACTAGGTCACCTGCCTGTTACTTGAGTTCTCAA | |
| | TCCTCTTGATCTCAACCTTCCCACGTCCC | |
| | GTGACCTAGTCCAAGAG | 5155 |
| | CTCTTGGACTAGGTCAC | 5156 |
| Male-sterile | CGTGGGAAGGTTGAGATCAAGAGGATTGAGAACTCAAGTAACAGG | 5157 |
| PI | CAGGTGACCTACTCCTAGAGGAGGAATGGGATTATCAAGAAGGCA | |
| Malus domestica | AAGGAGATCACTGTTCTATGTGATGCTAAAG | |
| Lys23Term | CTTTAGCATCACATAGAACAGTGATCTCCTTTGCCTTCTTGATAATC | 5158 |
| AAG-TAG | CCATTCCTCCTCTAGGAGTAGGTCACCTGCCTGTTACTTGAGTTCT | |
| | CAATCCTCTTGATCTCAACCTTCCCACG | |
| | CCTACTCCTAGAGGAGG | 5159 |
| | CCTCCTCTAGGAGTAGG | 5160 |
| Male-sterile | AGGATTGAGAACTCAAGTAACAGGCAGGTGACCTACTCCAAGAGG | 5161 |
| PI | AGGAATGGGATTATCTAGAAGGCAAAGGAGATCACTGTTCTATGT | |
| Malus domestica | GATGCTAAAGTATCTCTTATCATTTATTCTA | |
| Lys30Term | TAGAATAAATGATAAGAGATACTTTAGCATCACATAGAACAGTGAT | 5162 |
| AAG-TAG | CTCCTTTGCCTTCTAGATAATCCCATTCCTCCTCTTGGAGTAGGTC | |
| | ACCTGCCTGTTACTTGAGTTCTCAATCCT | |
| | GGATTATCTAGAAGGCA | 5163 |
| | TGCCTTCTAGATAATCC | 5164 |
| Male-sterile | ATTGAGAACTCAAGTAACAGGCAGGTGACCTACTCCAAGAGGAGG | 5165 |
| PI | AATGGGATTATCAAGTAGGCAAAGGAGATCACTGTTCTATGTGATG | |
| Malus domestica | CTAAAGTATCTCTTATCATTTATTCTAGCT | |
| Lys31Term | AGCTAGAATAAATGATAAGAGATACTTTAGCATCACATAGAACAGT | 5166 |
| AAG-TAG | GATCTCCTTTGCCTACTTGATAATCCCATTCCTCCTCTTGGAGTAG | |
| | GTCACCTGCCTGTTACTTGAGTTCTCAAT | |
| | TTATCAAGTAGGCAAAG | 5167 |
| | CTTTGCCTACTTGATAA | 5168 |
| Male-sterile | CATTTTTACAATAGTTATCTGCAAACAAAAACAAGAGAGAAAAACAA | 5169 |
| globosa | AAACAAAAAAATGTGAAGAGGAAAAATTGAGATCAAAAGAATTGAG | |
| Antirrhinum majus | AACTCAAGCAACAGGCAGGTTACTTACT | |
| Gly2Term | AGTAAGTAACCTGCCTGTTGCTTGAGTTCTCAATTCTTTTGATCTCA | 5170 |
| GGA-TGA | ATTTTTCCTCTTCACATTTTTTTGTTTTTGTTTTTCTCTCTTGTTTTTG | |
| | TTTGCAGATAACTATTGTAAAAATG | |
| | AAAAAATGTGAAGAGGA | 5171 |
| | TCCTCTTCAee CATTTTTT | 5172 |
| Male-sterile | TTTTACAATAGTTATCTGCAAACAAAAACAAGAGAGAAAAACAAAAA | 5173 |
| globosa | CAAAAAAATGGGATGAGGAAAAATTGAGATCAAAAGAATTGAGAAC | |
| Antirrhinum majus | TCAAGCAACAGGCAGGTTACTTACTCAA | |
| Arg3Term | TTGAGTAAGTAACCTGCCTGTTGCTTGAGTTCTCAATTCTTTTGATC | 5174 |
| AGA-TGA | TCAATTTTTCCTCATCCCATTTTTTTGTTTTTGTTTTTCTCTCTTGTTT | |
| | TTGTTTGCAGATAACTATTGTAAAA | |
| | AAATGGGATGAGGAAAA | 5175 |
| | TTTTCCTCATCCCATTT | 5176 |
| Male-sterile | TACAATAGTTATCTGCAAACAAAAACAAGAGAGAAAAACAAAAACA | 5177 |
| globosa | AAAAAATGGGAAGATGAAAAATTGAGATCAAAAGAATTGAGAACTC | |
| Antirrhinum majus | AAGCAACAGGCAGGTTACTTACTCAAAGA | |
| Gly4Term | TCTTTGAGTAAGTAACCTGCCTGTTGCTTGAGTTCTCAATTCTTTTG | 5178 |
| GGA-TGA | ATCTCAATTTTTCATCTTCCCATTTTTTTGTTTTTGTTTTTCTCTCTTG | |
| | TTTTTGTTTGCAGATAACTATTGTA | |
| | TGGGAAGATGAAAAATT | 5179 |
| | AATTTTTCATCTTCCCA | 5180 |
| Male-sterile | AATAGTTATCTGCAAACAAAAACAAGAGAGAAAAACAAAAACAAAA | 5181 |
| globosa | AAATGGGAAGAGGATAAATTGAGATCAAAAGAATTGAGAACTCAAG | |
| Antirrhinum majus | CAACAGGCAGGTTACTTACTCAAAGAGAA | |
| Lys5Term | TTCTCTTTGAGTAAGTAACCTGCCTGTTGCTTGAGTTCTCAATTCTT | 5182 |
| AAA-TAA | TTGATCTCAATTTATCCTCTTCCCATTTTTTTGTTTTTGTTTTTCTCT | |
| | CTTGTTTTTGTTTGCAGATAACTATT | |
| | GAAGAGGATAAATTGAG | 5183 |
| | CTCAATTTATCCTCTTC | 5184 |
| Male-sterile | GCTGAGCTCTTGCTGCCCTTGGATCTGTTTGGGAGTGGAGAACGC | 5185 |
| PI | AGTATGGGGCGCGGCTAGATCAAGATCAAGAGGATCGAGAACTCT | |
| Zea mays | ACCAACCGGCAGGTGACCTTCTCCAAGCGCC | |
| Lys5Term | GGCGCTTGGAGAAGGTCACCTGCCGGTTGGTAGAGTTCTCGATCC | 5186 |
| AAG-TAG | TCTTGATCTTGATCTAGCCGCGCCCCATACTGCGTTCTCCACTCCC | |
| | AAACAGATCCAAGGGCAGCAAGAGCTCAGC | |
| | GGCGCGGCTAGATCAAG | 5187 |
| | CTTGATCTAGCCGCGCC | 5188 |
| Male-sterile | CTCTTGCTGCCCTTGGATCTGTTTGGGAGTGGAGAACGCAGTATG | 5189 |
| PI | GGGCGCGGCAAGATCTAGATCAAGAGGATCGAGAACTCTACCAAC | |
| Zea mays | CGGCAGGTGACCTTCTCCAAGCGCCGGGCCG | |
| Lys7Term | CGGCCCGGCGCTTGGAGAAGGTCACCTGCCGGTTGGTAGAGTTC | 5190 |
| AAG-TAG | TCGATCCTCTTGATCTAGATCTTGCCGCGCCCCATACTGCGTTCTC | |
| | CACTCCCAAACAGATCCAAGGGCAGCAAGAG | |
| | GCAAGATCTAGATCAAG | 5191 |
| | CTTGATCTAGATCTTGC | 5192 |
| Male-sterile | CTCTTGCTGCCCTTGGATCTGTTTGGGAGTGGAGAACGCAGTATG | 5193 |
| PI | GGGCGCGGCAAGATCTAGATCAAGAGGATCGAGAACTCTACCAAC | |
| Zea mays | CGGCAGGTGACCTTCTCCAAGCGCCGGGCCG | |
| Lys9Term | CGGCCCGGCGCTTGGAGAAGGTCACCTGCCGGTTGGTAGAGTTC | 5194 |
| AAG-TAG | TCGATCCTCTTGATCTAGATCTTGCCGCGCCCCATACTGCGTTCTC | |
| | CACTCCCAAACAGATCCAAGGGCAGCAAGAG | |
| | GCAAGATCTAGATCAAG | 5195 |
| | CTTGATCTAGATCTTGC | 5196 |
| Male-sterile | GATCTGTTTGGGAGTGGAGAACGCAGTATGGGGCGCGGCAAGAT | 5197 |
| PI | CAAGATCAAGAGGATCTAGAACTCTACCAACCGGCAGGTGACCTT | |
| Zea mays | CTCCAAGCGCCGGGCCGGACTGGTCAAGAAGG | |
| Glu12Term | CCTTCTTGACCAGTCCGGCCCGGCGCTTGGAGAAGGTCACCTGC | 5198 |
| GAG-TAG | CGGTTGGTAGAGTTCTAGATCCTCTTGATCTTGATCTTGCCGCGCC | |
| | CCATACTGCGTTCTCCACTCCCAAACAGATC | |
| | AGAGGATCTAGAACTCT | 5199 |
| | AGAGTTCTAGATCCTCT | 5200 |
| Male-sterile | GCTGAGCTCTTGCTGCCCTTGAATCTGTTAGGGAGTGGAGAACGG | 5201 |
| PI | AGTATGGGGCGCGGCTAGATCGAGATCAAGAGGATCGAGAACTCT | |
| Zea mays | ACCAACCGGCAGGTGACCTTCTCCAAGCGCC | |
| Lys5Term | GGCGCTTGGAGAAGGTCACCTGCCGGTTGGTAGAGTTCTCGATCC | 5202 |
| AAG-TAG | TCTTGATCTCGATCTAGCCGCGCCCCATACTCCGTTCTCCACTCCC | |
| | TAACAGATTCAAGGGCAGCAAGAGCTCAGC | |
| | GGCGCGGCTAGATCGAG | 5203 |
| | CTCGATCTAGCCGCGCC | 5204 |
| Male-sterile | CTCTTGCTGCCCTTGAATCTGTTAGGGAGTGGAGAACGGAGTATG | 5205 |
| PI | GGGCGCGGCAAGATCTAGATCAAGAGGATCGAGAACTCTACCAAC | |
| Zea mays | CGGCAGGTGACCTTCTCCAAGCGCCGGGCCG | |
| Glu7Term | CGGCCCGGCGCTTGGAGAAGGTCACCTGCCGGTTGGTAGAGTTC | 5206 |
| GAG-TAG | TCGATCCTCTTGATCTAGATCTTGCCGCGCCCCATACTCCGTTCTC | |
| | CACTCCCTAACAGATTCAAGGGCAGCAAGAG | |
| | GCAAGATCTAGATCAAG | 5207 |
| | CTTGATCTAGATCTTGC | 5208 |
| Male-sterile | CTGCCCTTGAATCTGTTAGGGAGTGGAGAACGGAGTATGGGGCG | 5209 |
| PI | CGGCAAGATCGAGATCTAGAGGATCGAGAACTCTACCAACCGGCA | |
| Zea mays | GGTGACCTTCTCCAAGCGCCGGGCCGGACTGG | |
| Lys9Term | CCAGTCCGGCCCGGCGCTTGGAGAAGGTCACCTGCCGGTTGGTA | 5210 |
| AAG-TAG | GAGTTCTCGATCCTCTAGATCTCGATCTTGCCGCGCCCCATACTC | |
| | CGTTCTCCACTCCCTAACAGATTCAAGGGCAG | |
| | TCGAGATCTAGAGGATC | 5211 |
| | GATCCTCTAGATCTCGA | 5212 |
| Male-sterile | AATCTGTTAGGGAGTGGAGAACGGAGTATGGGGCGCGGCAAGAT | 5213 |
| PI | CGAGATCAAGAGGATCTAGAACTCTACCAACCGGCAGGTGACCTT | |
| Zea mays | CTCCAAGCGCCGGGCCGGACTGGTCAAGAAGG | |
| Glu12Term | CCTTCTTGACCAGTCCGGCCCGGCGCTTGGAGAAGGTCACCTGC | 5214 |
| GAG-TAG | CGGTTGGTAGAGTTCTAGATCCTCTTGATCTCGATCTTGCCGCGC | |
| | CCCATACTCCGTTCTCCACTCCCTAACAGATT | |
| | AGAGGATCTAGAACTCT | 5215 |
| | AGAGTTCTAGATCCTCT | 5216 |
| Male-sterile | TTGCTGCTAAGCTAGCTGGAGGAAGGAGGAGGAGGAGGAGGAG | 5217 |
| PI | CGGGATGGGGCGCGGGTAGATCGAGATCAAGAGGATCGAGAACT | |
| Oryza sativa | CCACCAACCGCCAGGTGACCTTCTCCAAGCGCA | |
| Lys5Term | TGCGCTTGGAGAAGGTCACCTGGCGGTTGGTGGAGTTCTCGATCC | 5218 |
| AAG-TAG | TCTTGATCTCGATCTACCCGCGCCCCATCCCGCCTCCTCCTCCTC | |
| | CTCCTCCTTCCTCCAGCTAGCTTAGCAGCAA | |
| | GGCGCGGGTAGATCGAG | 5219 |
| | CTCGATCTACCCGCGCC | 5220 |
| Male-sterile | CTAAGCTAGCTGGAGGAAGGAGGAGGAGGAGGAGGAGGCGGGA | 5221 |
| PI | TGGGGCGCGGGAAGATCTAGATCAAGAGGATCGAGAACTCCACC | |
| Oryza sativa | AACCGCCAGGTGACCTTCTCCAAGCGCAGGAGCG | |
| Glu7Term | CGCTCCTGCGCTTGGAGAAGGTCACCTGGCGGTTGGTGGAGTTCT | 5222 |
| GAG-TAG | CGATCCTCTTGATCTAGATCTTCCCGCGCCCCATCCCGCCTCCTC | |
| | CTCCTCCTCCTCCTTCCTCCAGCTAGCTTAG | |
| | GGAAGATCTAGATCAAG | 5223 |
| | CTTGATCTAGATCTTCC | 5224 |
| Male-sterile | TAGCTGGAGGAAGGAGGAGGAGGAGGAGGAGGCGGGATGGGGC | 5225 |
| PI | GCGGGAAGATCGAGATCTAGAGGATCGAGAACTCCACCAACCGC | |
| Oryza sativa | CAGGTGACCTTCTCCAAGCGCAGGAGCGGGATCC | |
| Lys9Term | GGATCCCGCTCCTGCGCTTGGAGAAGGTCACCTGGCGGTTGGTG | 5226 |
| AAG-TAG | GAGTTCTCGATCCTCTAGATCTCGATCTTCCCGCGCCCCATCCCG | |
| | CCTCCTCCTCCTCCTCCTCCTTCCTCCAGCTA | |
| | TCGAGATCTAGAGGATC | 5227 |
| | GATCCTCTAGATCTCGA | 5228 |
| Male-sterile | GAAGGAGGAGGAGGAGGAGGAGGCGGGATGGGGCGCGGGAAG | 5229 |
| PI | ATCGAGATCAAGAGGATCTAGAACTCCACCAACCGCCAGGTGACC | |
| Oryza sativa | TTCTCCAAGCGCAGGAGCGGGATCCTCAAGAAGG | |
| Glu12Term | CCTTCTTGAGGATCCCGCTCCTGCGCTTGGAGAAGGTCACCTGGC | 5230 |
| GAG-TAG | GGTTGGTGGAGTTCTAGATCCTCTTGATCTCGATCTTCCCGCGCC | |
| | CCATCCCGCCTCCTCCTCCTCCTCCTCCTTC | |
| | AGAGGATCTAGAACTCC | 5231 |
| | GGAGTTCTAGATCCTCT | 5232 |

### EXAMPLE 28

### Engineering Plants for Abiotic Stress Tolerance

Environmental stresses, such as drought, increased soil salinity, soil contamination with heavy metals, and extreme temperature, are major factors limiting plant growth and productivity. The worldwide loss in yield of three major cereal crops, rice, maize, and wheat due to water stress (drought) has been estimated to be over ten billion dollars annually and many currently marginal soils could be brought into cultivation if suitable plant varieties were available.

Physiological and biochemical responses to high levels of ionic or nonionic solutes and decreased water potential have been studied in a variety of plants. It is known, for example, that increasing levels of alcohol dehydrogenase can confer enhances flooding resistance in plants. There are also several possible mechanisms to enhance plant salt tolerance. For example, one mechanism underlying the adaptation or tolerance of plants to osmotic stresses is the accumulation of compatible, low molecular weight osmolytes such as sugar alcohols, special amino acids, and glycinebetaine. Such accumulation can be engineered, for example, by removing feedback inhibition on 1-pyrroline-t-carboxylate synthetase, which results in accumulation of proline. Additionally, recent experiments suggest that altering the expression or activity of specific sodium or potassium transporters can confer enhanced salt tolerance.

Plant tolerance of contamination by heavy metals such as lead and aluminum in soils has also been investigated and one mechanism underlying tolerance is the production of dicarboxylic acids such as oxalate and citrate. In addition, individual genes involved in heavy metal sensitivity have been identified.

The attached tables disclose exemplary oligonucleotide base sequences which can be used to generate site-specific mutations that confer stress tolerance in plants.

TABLE 35
Genome-Altering Oligos Conferring Stress Tolerance

| Phenotype, Gene, | | |
|---|---|---|
| Plant & Targeted | | SEQ ID |
| Alteration | Altering Oligos | NO: |
| Salt Tolerance | CGTCTTTTTGTGTGGTAGTTGGATGTGACGGTTGCTCAAATGCTT | 5233 |
| P5CS | GTGACCGATAGCAGTGCTAGAGATAAGGATTTCAGGAAGCAACTT | |
| Arabidopsis thaliana | AGTGAAACTGTCAAAGCGATGCTGAGGATGA | |
| Phe128Ala | TCATCCTCAGCATCGCTTTGACAGTTTCACTAAGTTGCTTCCTGAA | 5234 |
| TTT-GCT | ATCCTTATCTCTAGCACTGCTATCGGTCACAAGCATTTGAGCAACC | |
| | GTCACATCCAACTACCACACAAAAAGACG | |
| | ATAGCAGTGCTAGAGAT | 5235 |
| | ATCTCTAGCACTGCTAT | 5236 |
| Salt Tolerance | GAGACTATGTTTGACCAGCTGGATGTGACGGCTGCTCAGCTGCTG | 5237 |
| P5CS 1 | GTGAATGACAGTAGTGCCAGAGACAAGGAGTTCAGGAAGCAACTT | |
| Brassica napus | AATGAGACAGTGAAGTCCATGCTTGATTTGA | |
| Phe128Ala | TCAAATCAAGCATGGACTTCACTGTCTCATTAAGTTGCTTCCTGAA | 5238 |
| TTC-GCC | CTCCTTGTCTCTGGCACTACTGTCATTCACCAGCAGCTGAGCAGC | |
| | CGTCACATCCAGCTGGTCAAACATAGTCTC | |
| | ACAGTAGTGCCAGAGAC | 5239 |
| | GTCTCTGGCACTACTGT | 5240 |
| Salt Tolerance | GAGACTATGTTTGACCAGATGGATGTGACGGTGGCTCAAATGCTG | 5241 |
| P5CS 2 | GTGACTGATAGCAGTGTCAGAGATAAGGATTTCAGGAAGCAACTT | |
| Brassica napus | AGTGAGACAGTCAAAGCTATGCTGAAAATGA | |
| Phe129Ala | TCATTTTCAGCATAGCTTTGACTGTCTCACTAAGTTGCTTCCTGAA | 5242 |
| TTC-GCC | ATCCTTATCTCTGACACTGCTATCAGTCACCAGCATTTGAGCCACC | |
| | GTCACATCCATCTGGTCAAACATAGTCTC | |
| | ATAGCAGTGTCAGAGAT | 5243 |
| | ATCTCTGACACTGCTAT | 5244 |
| Salt Tolerance | GATATGTTGTTTAACCAACTGGATGTCTCGTCATCTCAACTTCTTG | 5245 |
| P5CS | TCACCGACAGTGATGCTGAGAACCCAAAGTTCCGGGAGCAACTCA | |
| Oryza sativa | CTGAAACTGTTGAGTCATTATTAGATCTTA | |
| Phe128Ala | TAAGATCTAATAATGACTCAACAGTTTCAGTGAGTTGCTCCCGGAA | 5246 |
| TTT-GCT | CTTTGGGTTCTCAGCATCACTGTCGGTGACAAGAAGTTGAGATGA | |
| | CGAGACATCCAGTTGGTTAAACAACATATC | |
| | ACAGTGATGCTGAGAAC | 5247 |
| | GTTCTCAGCATCACTGT | 5248 |
| Salt Tolerance | GATATTTTGTTTAGTCAGCTGGATGTGACATCTGCTCAGCTTCTTG | 5249 |
| P5CS | TTACTGACAATGATGCTAGAGACCAAGATTTTAGAAAGCAACTTTC | |
| Medicago sativa | TGAAACTGTGAGATCACTTCTAGCACTAA | |
| Phe128Ala | TTAGTGCTAGAAGTGATCTCACAGTTTCAGAAAGTTGCTTTCTAAA | 5250 |
| TTT-GCT | ATCTTGGTCTCTAGCATCATTGTCAGTAACAAGAAGCTGAGCAGAT | |
| | GTCACATCCAGCTGACTAAACAAAATATC | |
| | ACAATGATGCTAGAGAC | 5251 |
| | GTCTCTAGCATCATTGT | 5252 |
| Salt Tolerance | GATACATTGTTTAGTCAGCTGGATGTGACATCAGCTCAGCTACTC | 5253 |
| P5CS | GTTACTGATAATGATGCTAGGGATCCAGAATTCAGGAAGCAACTT | |
| Actinidia deliciosa | ACTGAAACTGTAGAATCACTATTGAATTTGA | |
| Phe128Ala | TCAAATTCAATAGTGATTCTACAGTTTCAGTAAGTTGCTTCCTGAAT | 5254 |
| TTT-GCT | TCTGGATCCCTAGCATCATTATCAGTAACGAGTAGCTGAGCTGAT | |
| | GTCACATCCAGCTGACTAAACAATGTATC | |
| | ATAATGATGCTAGGGAT | 5255 |
| | ATCCCTAGCATCATTAT | 5256 |
| Salt Tolerance | GACACACTCTTCAGTCAACTGGATGTGACATCAGCACAGCTTCTT | 5257 |
| P5CS | GTAACAGATAATGACGCCAGAAGTCCAGAATTTAGAAAACAACTTA | |
| Cichorium intybus | CTGAAACAGTCGATTCTTTATTATCTTATA | |
| Phe122Ala | TATAAGATAATAAAGAATCGACTGTTTCAGTAAGTTGTTTTCTAAAT | 5258 |
| TTC-GCC | TCTGGACTTCTGGCGTCATTATCTGTTACAAGAAGCTGTGCTGAT | |
| | GTCACATCCAGTTGACTGAAGAGTGTGTC | |
| | ATAATGACGCCAGAAGT | 5259 |
| | ACTTCTGGCGTCATTAT | 5260 |
| Salt Tolerance | GATTCTTTGTTCAGTCAGTTGGATGTGACATCAGCTCAGCTTCTGG | 5261 |
| P5CS | TGACTGATAATGACGCTAGAGATCCAGATTTTAGGAGACAACTCA | |
| Lycopersicon | ATGACACAGTAAATTCGTTGCTTTCTCTAA | |
| esculentum | TTAGAGAAAGCAACGAATTTACTGTGTCATTGAGTTGTCTCCTAAA | 5262 |
| Phe128Ala | ATCTGGATCTCTAGCGTCATTATCAGTCACCAGAAGCTGAGCTGA | |
| TTT-GCT | TGTCACATCCAACTGACTGAACAAAGAATC | |
| | ATAATGACGCTAGAGAT | 5263 |
| | ATCTCTAGCGTCATTAT | 5264 |
| Salt Tolerance | GATACCATGTTCAGCCAGCTTGATGTGACTTCTTCCCAACTTCTTG | 5265 |
| P5CS | TGAATGATGGATTTGCTAGGGATGCTGGCTTCAGAAAACAACTTT | |
| Vigna unguiculata | CGGACACAGTGAACGCGTTATTAGATTTAA | |
| Phe162Ala | TTAAATCTAATAACGCGTTCACTGTGTCCGAAAGTTGTTTTCTGAA | 5266 |
| TTT-GCT | GCCAGCATCCCTAGCAAATCCATCATTCACAAGAAGTTGGGAAGA | |
| | AGTCACATCAAGCTGGCTGAACATGGTATC | |
| | ATGGATTTGCTAGGGAT | 5267 |
| | ATCCCTAGCAAATCCAT | 5268 |
| Salt Tolerance | GACACCTTGTTTAGTCAGTTGGATCTGACTGCTGCTCAGCTGCTT | 5269 |
| P5CS | GTGACGGACAACGACGCTAGAGATCCAAGTTTTAGAACACAACTA | |
| Mesembryanthemum | ACTGAAACAGTGTATCAGTTGTTGGATCTAA | |
| crystallinum | TTAGATCCAACAACTGATACACTGTTTCAGTTAGTTGTGTTCTAAA | 5270 |
| Phe125Ala | ACTTGGATCTCTAGCGTCGTTGTCCGTCACAAGCAGCTGAGCAGC | |
| TTT-GCT | AGTCAGATCCAACTGACTAAACAAGGTGTC | |
| | ACAACGACGCTAGAGAT | 5271 |
| | ATCTCTAGCGTCGTTGT | 5272 |
| Salt Tolerance | GACACATTATTTAGCCAGCTGGATGTGACATCAGCTCAGCTTCTT | 5273 |
| P5CS | GTGACTGATAATGATGCTAGGGATGAAGCTTTCCGAAATCAACTTA | |
| Vitis vinifera | CTCAAACAGTGGATTCATTGTTAGCTTTGA | |
| Phe130Ala | TCAAAGCTAACAATGAATCCACTGTTTGAGTAAGTTGATTTCGGAA | 5274 |
| TTT-GCT | AGCTTCATCCCTAGCATCATTATCAGTCACAAGAAGCTGAGCTGAT | |
| | GTCACATCCAGCTGGCTAAATAATGTGTC | |
| | ATAATGATGCTAGGGAT | 5275 |
| | ATCCCTAGCATCATTAT | 5276 |
| Salt Tolerance | GATACGCTGTTCACTCAGCTCGATGTGACATCGGCTCAGCTTCTT | 5277 |
| P5CS | GTGACGGATAACGATGCTCGAGATAAGGATTTCAGGAAGCAGCTT | |
| Vigna aconitifolia | ACTGAGACTGTGAAGTCGCTGTTGGCGCTGA | |
| Phe129Ala | TCAGCGCCAACAGCGACTTCACAGTCTCAGTAAGCTGCTTCCTGA | 5278 |
| TTT-GCT | AATCCTTATCTCGAGCATCGTTATCCGTCACAAGAAGCTGAGCCG | |
| | ATGTCACATCGAGCTGAGTGAACAGCGTATC | |
| | ATAACGATGCTCGAGAT | 5279 |
| | ATCTCGAGCATCGTTAT | 5280 |
| Salt Tolerance | AGAGATGTTCTTAGTTCCAAAGAAATCTCACCTCTCACTTTCTCCG | 5281 |
| HKT1 | TCTTCACAACAGTTGTCACGTTTGCAAACTGCGGATTTGTCCCCAC | |
| Arabidopsis thaliana | GAATGAGAACATGATCATCTTTCGCAAAA | |
| Ser207Val | TTTTGCGAAAGATGATCATGTTCTCATTCGTGGGGACAAATCCGC | 5282 |
| TCC-GTC | AGTTTGCAAACGTGACAACTGTTGTGAAGACGGAGAAAGTGAGAG | |
| | GTGAGATTTCTTTGGAACTAAGAACATCTCT | |
| | CAACAGTTGTCACGTTT | 5283 |
| | AAACGTGACAACTGTTG | 5284 |
| Salt Tolerance | CGAATGAGAACATGATCATCTTTCGCAAAAACTCTGGTCTCATCTG | 5285 |
| HKT1 | GCTCCTAATCCCTCTAGTACTGATGGGAAACACTTTGTTCCCTTGC | |
| Arabidopsis thaliana | TTCTTGGTTTTGCTCATATGGGGACTTTA | |
| Gln237Leu | TAAAGTCCCCATATGAGCAAAACCAAGAAGCAAGGGAACAAAGTG | 5286 |
| CAA-CTA | TTTCCCATCAGTACTAGAGGGATTAGGAGCCAGATGAGACCAGAG | |
| | TTTTTGCGAAAGATGATCATGTTCTCATTCG | |
| | AATCCCTCTAGTACTGA | 5287 |
| | TCAGTACTAGAGGGATT | 5288 |
| Salt Tolerance | AGTCTCTAGAAGGAATGAGTTCGTACGAGAAGTTGGTTGGATCGT | 5289 |
| HKT1 | TGTTTCAAGTGGTGAGTTCGCGACACACCGGAGAAACTATAGTAG | |
| Arabidopsis thaliana | ACCTCTCTACACTTTCCCCAGCTATCTTGGT | |
| Asn332Ser | ACCAAGATAGCTGGGGAAAGTGTAGAGAGGTCTACTATAGTTTCT | 5290 |
| AAT-AGT | CCGGTGTGTCGCGAACTCACCACTTGAAACAACGATCCAACCAAC | |
| | TTCTCGTACGAACTCATTCCTTCTAGAGACT | |
| | AGTGGTGAGTTCGCGAC | 5291 |
| | GTCGCGAACTCACCACT | 5292 |
| Salt Tolerance | AGAGATGTGCTAAAGAAGAAAGGTCTCAAAATGGTGACCTTTTCC | 5293 |
| HKT1 | GTCTTCACCACCGTGGTGACCTTTGCCAGTTGTGGGTTTGTCCCG | |
| Eucalyptus | ACCAATGAAAACATGATTATCTTCAGCAAAA | |
| camaldulensis | TTTTGCTGAAGATAATCATGTTTTCATTGGTCGGGACAAACCCACA | 5294 |
| Ser256Val | ACTGGCAAAGGTCACCACGGTGGTGAAGACGGAAAAGGTCACCA | |
| TCG-GTG | TTTTGAGACCTTTCTTCTTTAGCACATCTCT | |
| | CCACCGTGGTGACCTTT | 5295 |
| | AAAGGTCACCACGGTGG | 5296 |
| Salt Tolerance | CCAATGAAAACATGATTATCTTCAGCAAAAACTCTGGCCTCCTCCT | 5297 |
| HKT1 | GATTCTCATCCCTCTGGCCCTTCTTGGGAACATGCTGTTCCCATC | |
| Eucalyptus | GAGCCTACGTTTGACGCTTTGGCTCATCGG | |
| camaldulensis | CCGATGAGCCAAAGCGTCAAACGTAGGCTCGATGGGAACAGCAT | 5298 |
| Gln286Leu | GTTCCCAAGAAGGGCCAGAGGGATGAGAATCAGGAGGAGGCCA | |
| CAG-CTG | GAGTTTTTGCTGAAGATAATCATGTTTTCATTGG | |
| | CATCCCTCTpee GGCCCTTC | 5299 |
| | GAAGGGCCAGAGGGATG | 5300 |
| Salt Tolerance | AATCGTTGAATGGACTAAGCTCCTGTGAGAAAATCGTGGGCGCGC | 5301 |
| HKT1 | TGTTTCAGTGCGTGAGCAGCAGACATACCGGCGAGACGGTCGTC | |
| Eucalyptus | GATCTGTCCACAGTTGCTCCCGCCATCTTGGT | |
| camaldulensis | ACCAAGATGGCGGGAGCAACTGTGGACAGATCGACGACCGTCTC | 5302 |
| Asn381Ser | GCCGGTATGTCTGCTGCTCACGCACTGAAACAGCGCGCCCACGA | |
| AAC-AGC | TTTTCTCACAGGAGCTTAGTCCATTCAACGATT | |
| | GTGCGTGAGCAGCAGAC | 5303 |
| | GTCTGCTGCTCACGCAC | 5304 |
| Salt Tolerance | AAAGCTCCACTGAAGAAGAAAGGGATCAACATTGCACTCTTCTCA | 5305 |
| HKT1 | TTCTCGGTCACGGTCGTCTCGTTTGCGAATGTGGGGCTCGTGCC | |
| Oryza sativa | GACAAATGAGAACATGGCAATCTTCTCCAAGA | |
| Ser238Val | TCTTGGAGAAGATTGCCATGTTCTCATTTGTCGGCACGAGCCCCA | 5306 |
| TCC-GTC | CATTCGCAAACGAGACGACCGTGACCGAGAATGAGAAGAGTGCA | |
| | ATGTTGATCCCTTTCTTCTTCAGTGGAGCTTT | |
| | TCACGGTCGTCTCGTTT | 5307 |
| | AAACGAGACGACCGTGA | 5308 |
| Salt Tolerance | CAAATGAGAACATGGCAATCTTCTCCAAGAACCCGGGCCTCCTCC | 5309 |
| HKT1 | TCCTGTTCATCGGCCTGATTCTTGCAGGCAATACACTTTACCCTCT | |
| Oryza sativa | CTTCCTAAGGCTATTGATATGGTTCCTGGG | |
| Gln268Leu | CCCAGGAACCATATCAATAGCCTTAGGAAGAGAGGGTAAAGTGTA | 5310 |
| CAG-CTG | TTGCCTGCAAGAATCAGGCCGATGAACAGGAGGAGGAGGCCCGG | |
| | GTTCTTGGAGAAGATTGCCATGTTCTCATTTG | |
| | CATCGGCCTGATTCTTG | 5311 |
| | CAAGAATCAGGCCGATG | 5312 |
| Salt Tolerance | CAGTCTTTGATGGACTCAGCTCTTACCAGAAGATTATCAATGCATT | 5313 |
| HKT1 | GTTCATGGCAGTGAGCGCAAGGCACTCGGGGGAGAACTCCATCG | |
| Oryza sativa | ACTGCTCACTCATCGCCCCTGCTGTTCTAGT | |
| Asn363Ser | ACTAGAACAGCAGGGGCGATGAGTGAGCAGTCGATGGAGTTCTC | 5314 |
| AAC-AGC | CCCCGAGTGCCTTGCGCTCACTGCCATGAACAATGCATTGATAAT | |
| | CTTCTGGTAAGAGCTGAGTCCATCAAAGACTG | |
| | GGCAGTGAGCGCAAGGC | 5315 |
| | GCCTTGCGCTCACTGCC | 5316 |
| Salt Tolerance | GTGCCCCACTGAACAAGAAAGGGATCAACATCGTGCTCTTCTCAC | 5317 |
| HKT1 | TATCAGTCACCGTTGTCTCCTGTGCGAATGCAGGACTCGTGCCCA | |
| Triticum aestivum | CAAATGAGAACATGGTCATCTTCTCAAAGAA | |
| Ala240Val | TTCTTTGAGAAGATGACCATGTTCTCATTTGTGGGCACGAGTCCT | 5318 |
| GCC-GTC | GCATTCGCACAGGAGACAACGGTGACTGATAGTGAGAAGAGCAC | |
| | GATGTTGATCCCTTTCTTGTTCAGTGGGGCAC | |
| | CACCGTTGTCTCCTGTG | 5319 |
| | CACAGGAGACAACGGTG | 5320 |
| Salt Tolerance | CAAATGAGAACATGGTCATCTTCTCAAAGAATTCAGGCCTCTTGTT | 5321 |
| HKT1 | GCTGCTGAGTGGCCTGATGCTCGCAGGCAATACATTGTTCCCTCT | |
| Triticum aestivum | CTTCCTGAGGCTACTGGTGTGGTTCCTGGG | |
| Gln270Leu | CCCAGGAACCACACCAGTAGCCTCAGGAAGAGAGGGAACAATGT | 5322 |
| CAG-CTG | ATTGCCTGCGAGCATCAGGCCACTCAGCAGCAACAAGAGGCCTG | |
| | AATTCTTTGAGAAGATGACCATGTTCTCATTTG | |
| | GAGTGGCCTGATGCTCG | 5323 |
| | CGAGCATCAGGCCACTC | 5324 |
| Salt Tolerance | CAGTCTTTGATGGGCTCAGCTCTTATCAGAAGACTGTCAATGCATT | 5325 |
| HKT1 | CTTCATGGTGGTGAGTGCGAGGCACTCAGGGGAGAATTCCATCG | |
| Triticum aestivum | ACTGCTCGCTCATGTCCCCTGCCATTATAGT | |
| Asn365Ser | ACTATAATGGCAGGGGACATGAGCGAGCAGTCGATGGAATTCTCC | 5326 |
| AAT-AGT | CCTGAGTGCCTCGCACTCACCACCATGAAGAATGCATTGACAGTC | |
| | TTCTGATAAGAGCTGAGCCCATCAAAGACTG | |
| | GGTGGTGAGTGCGAGGC | 5327 |
| | GCCTCGCACTCACCACC | 5328 |
| Freezing Tolerance | TTTTTTTTGTTTTCGTTTTCAAAAACAAAATCTTTGAATTTTATGGCA | 5329 |
| proline oxidase | ACCCGTCTTCTCTGAACAAACTTTATCCGGCGATCTTACCGTTTAC | |
| precursor | CCGCTTTTAGCCCGGTGGGTCCTCCCA | |
| Arabidopsis thaliana | TGGGAGGACCCACCGGGCTAAAAGCGGGTAAACGGTAAGATCGC | 5330 |
| Arg7Term | CGGATAAAGTTTGTTCAGAGAAGACGGGTTGCCATAAAATTCAAA | |
| CGA-TGA | GATTTTGTTTTTGAAAACGAAAACAAAAAAAA | |
| | GTCTTCTCTGAACAAAC | 5331 |
| | GTTTGTTCAGAGAAGAC | 5332 |
| Freezing Tolerance | TCAAAAACAAAATCTTTGAATTTTATGGCAACCCGTCTTCTCAGAA | 5333 |
| proline oxidase | CAAACTTTATCCGGTGATCTTACCGTTTACCCGCTTTTAGCCCGGT | |
| precursor | GGGTCCTCCCACCGTGACTGCTTCCACCG | |
| Arabidopsis thaliana | CGGTGGAAGCAGTCACGGTGGGAGGACCCACCGGGCTAAAAGC | 5334 |
| Arg13Term | GGGTAAACGGTAAGATCACCGGATAAAGTTTGTTCTGAGAAGACG | |
| CGA-TGA | GGTTGCCATAAAATTCAAAGATTTTGTTTTTGA | |
| | TTATCCGGTGATCTTAC | 5335 |
| | GTAAGATCACCGGATAA | 5336 |
| Freezing Tolerance | AAAATCTTTGAATTTTATGGCAACCCGTCTTCTCCGAACAAACTTT | 5337 |
| proline oxidase | ATCCGGCGATCTTAGCGTTTACCCGCTTTTAGCCCGGTGGGTCCT | |
| precursor | CCCACCGTGACTGCTTCCACCGCCGTCGTC | |
| Arabidopsis thaliana | GACGACGGCGGTGGAAGCAGTCACGGTGGGAGGACCCACCGGG | 5338 |
| Tyr15Term | CTAAAAGCGGGTAAACGCTAAGATCGCCGGATAAAGTTTGTTCGG | |
| TAC-TAG | AGAAGACGGGTTGCCATAAAATTCAAAGATTTT | |
| | CGATCTTAGCGTTTACC | 5339 |
| | GGTAAACGCTAAGATCG | 5340 |
| Freezing Tolerance | CTTTGAATTTTATGGCAACCCGTCTTCTCCGAACAAACTTTATCCG | 5341 |
| proline oxidase | GCGATCTTACCGTTAACCCGCTTTTAGCCCGGTGGGTCCTCCCAC | |
| precursor | CGTGACTGCTTCCACCGCCGTCGTCCCGGA | |
| Arabidopsis thaliana | TCCGGGACGACGGCGGTGGAAGCAGTCACGGTGGGAGGACCCA | 5342 |
| Leu17Term | CCGGGCTAAAAGCGGGTTAACGGTAAGATCGCCGGATAAAGTTT | |
| TTA-TAA | GTTCGGAGAAGACGGGTTGCCATAAAATTCAAAG | |
| | TTACCGTTAACCCGCTT | 5343 |
| | AAGCGGGTTAACGGTAA | 5344 |
| Freezing Tolerance | CCGGTGGGTCCTCCCACCGTGACTGCTTCCACCGCCGTCGTCCC | 5345 |
| proline oxidase | GGAGATTCTCTCCTTTTGACAACAAGCACCGGAACCACCTCTTCA | |
| precursor | CCACCCAAAACCCACCGAGCAATCTCACGATG | |
| Arabidopsis thaliana | CATCGTGAGATTGCTCGGTGGGTTTTGGGTGGTGAAGAGGTGGT | 5346 |
| Gly42Term | TCCGGTGCTTGTTGTCAAAAGGAGAGAATCTCCGGGACGACGGC | |
| GGA-TGA | GGTGGAAGCAGTCACGGTGGGAGGACCCACCGG | |
| | TCTCCTTTTGACAACAA | 5347 |
| | TTGTTGTCAAAAGGAGA | 5348 |
| Lead Tolerance | ACATGAAGCAGTGAAATCTCTGTTTGTATTGAATCTTATTAGTCTC | 5349 |
| cyclic nucleotide- | AAACTATGAATTTCTGACAAGAGAAGTTTGTAAGGTCAGTGTTCCA | |
| regulated ion channel | GATTTGTCTCATTGAATTCTAAGTCGTGA | |
| Arabidopsis thaliana | TCACGACTTAGAATTCAATGAGACAAATCTGGAACACTGACCTTAC | 5350 |
| Arg4Term | AAACTTCTCTTGTCAGAAATTCATAGTTTGAGACTAATAAGATTCAA | |
| CGA-TGA | TACAAACAGAGATTTCACTGCTTCATGT | |
| | TGAATTTCTGACAAGAG | 5351 |
| | CTCTTGTCAGAAATTCA | 5352 |
| Lead Tolerance | TGAAGCAGTGAAATCTCTGTTTGTATTGAATCTTATTAGTCTCAAA | 5353 |
| cyclic nucleotide- | CTATGAATTTCCGATAAGAGAAGTTTGTAAGGTCAGTGTTCCAGAT | |
| regulated ion channel | TTGTCTCATTGAATTCTAAGTCGTGAAGC | |
| Arabidopsis thaliana | GCTTCACGACTTAGAATTCAATGAGACAAATCTGGAACACTGACCT | 5354 |
| Gln5Term | TACAAACTTCTCTTATCGGAAATTCATAGTTTGAGACTAATAAGATT | |
| CAA-TAA | CAATACAAACAGAGATTTCACTGCTTCA | |
| | ATTTCCGATAAGAGAAG | 5355 |
| | CTTCTCTTATCGGAAAT | 5356 |
| Lead Tolerance | AGCAGTGAAATCTCTGTTTGTATTGAATCTTATTAGTCTCAAACTAT | 5357 |
| cyclic nucleotide- | GAATTTCCGACAATAGAAGTTTGTAAGGTCAGTGTTCCAGATTTGT | |
| regulated ion channel | CTCATTGAATTCTAAGTCGTGAAGCTTA | |
| Arabidopsis thaliana | TAAGCTTCACGACTTAGAATTCAATGAGACAAATCTGGAACACTGA | 5358 |
| Glu6Term | CCTTACAAACTTCTATTGTCGGAAATTCATAGTTTGAGACTAATAA | |
| GAG-TAG | GATTCAATACAAACAGAGATTTCACTGCT | |
| | TCCGACAATAGAAGTTT | 5359 |
| | AAACTTCTATTGTCGGA | 5360 |
| Lead Tolerance | AGTGAAATCTCTGTTTGTATTGAATCTTATTAGTCTCAAACTATGAA | 5361 |
| cyclic nucleotide- | TTTCCGACAAGAGTAGTTTGTAAGGTCAGTGTTCCAGATTTGTCTC | |
| regulated ion channel | ATTGAATTCTAAGTCGTGAAGCTTAATT | |
| Arabidopsis thaliana | AATTAAGCTTCACGACTTAGAATTCAATGAGACAAATCTGGAACAC | 5362 |
| Lys7Term | TGACCTTACAAACTACTCTTGTCGGAAATTCATAGTTTGAGACTAA | |
| AAG-TAG | TAAGATTCAATACAAACAGAGATTTCACT | |
| | GACAAGAGTAGTTTGTA | 5363 |
| | TACAAACTACTCTTGTC | 5364 |
| Lead Tolerance | CATTGAATTCTAAGTCGTGAAGCTTAATTCGATTCTTCTTCACTTTC | 5365 |
| cyclic nucleotide- | TCGGATCAGGTTTTAAGATTGGAAGTCGGATAAGACTTCCTCCGA | |
| regulated ion channel | CGTGGAATATTCCGGTAAAAACGAGATTC | |
| Arabidopsis thaliana | GAATCTCGTTTTTACCGGAATATTCCACGTCGGAGGAAGTCTTATC | 5366 |
| Gln12Term | CGACTTCCAATCTTAAAACCTGATCCGAGAAAGTGAAGAAGAATC | |
| CAA-TAA | GAATTAAGCTTCACGACTTAGAATTCAATG | |
| | TCAGGTTTTAAGATTGG | 5367 |
| | CCAATCTTAAAACCTGA | 5368 |
| Lead Tolerance | TGGAAGTCAATCCCCCACGTTGAGCAGGTTGATGCATTGGCTAAA | 5369 |
| cyclic nucleotide- | GTTATGAATCACCGCTAAGACGAGTTTGTGAGGTTTCAGGATTGG | |
| gated calmodulin- | AAATCAGAGAGAAGCTCTGAGGGAAATTTTC | |
| binding ion channel | GAAAATTTCCCTCAGAGCTTCTCTCTGATTTCCAATCCTGAAACCT | 5370 |
| (CBP4) | CACAAACTCGTCTTAGCGGTGATTCATAACTTTAGCCAATGCATCA | |
| Nicotiana Tabacum | ACCTGCTCAACGTGGGGGATTGACTTCCA | |
| Gln5Term | ATCACCGCTAAGACGAG | 5371 |
| CAA-TAA | CTCGTCTTAGCGGTGAT | 5372 |
| Lead Tolerance | TCAATCCCCCACGTTGAGCAGGTTGATGCATTGGCTAAAGTTATG | 5373 |
| cyclic nucleotide- | AATCACCGCCAAGACTAGTTTGTGAGGTTTCAGGATTGGAAATCA | |
| gated calmodulin- | GAGAGAAGCTCTGAGGGAAATTTTCATGCTA | |
| binding ion channel | TAGCATGAAAATTTCCCTCAGAGCTTCTCTCTGATTTCCAATCCTG | 5374 |
| (CBP4) | AAACCTCACAAACTAGTCTTGGCGGTGATTCATAACTTTAGCCAAT | |
| Nicotiana Tabacum | GCATCAACCTGCTCAACGTGGGGGATTGA | |
| Gly7Term | GCCAAGACTAGTTTGTG | 5375 |
| GAG-TAG | CACAAACTAGTCTTGGC | 5376 |
| Lead Tolerance | GAGCAGGTTGATGCATTGGCTAAAGTTATGAATCACCGCCAAGAC | 5377 |
| cyclic nucleotide- | GAGTTTGTGAGGTTTTAGGATTGGAAATCAGAGAGAAGCTCTGAG | |
| gated calmodulin- | GGAAATTTTCATGCTAAAGGTGGAGTCCACC | |
| binding ion channel | GGTGGACTCCACCTTTAGCATGAAAATTTCCCTCAGAGCTTCTCTC | 5378 |
| (CBP4) | TGATTTCCAATCCTAAAACCTCACAAACTCGTCTTGGCGGTGATTC | |
| Nicotiana Tabacum | ATAACTTTAGCCAATGCATCAACCTGCTC | |
| Gln12Term | TGAGGTTTTAGGATTGG | 5379 |
| CAG-TAG | CCAATCCTAAAACCTCA | 5380 |
| Lead Tolerance | TGATGCATTGGCTAAAGTTATGAATCACCGCCAAGACGAGTTTGT | 5381 |
| cyclic nucleotide- | GAGGTTTCAGGATTGTAAATCAGAGAGAAGCTCTGAGGGAAATTT | |
| gated calmodulin- | TCATGCTAAAGGTGGAGTCCACCGAAGTAAA | |
| binding ion channel | TTTACTTCGGTGGACTCCACCTTTAGCATGAAAATTTCCCTCAGAG | 5382 |
| (CBP4) | CTTCTCTCTGATTTACAATCCTGAAACCTCACAAACTCGTCTTGGC | |
| Nicotiana Tabacum | GGTGATTCATAACTTTAGCCAATGCATCA | |
| Trp14Term | CAGGATTGTAAATCAGA | 5383 |
| TGG-TGA | TCTGATTTACAATCCTG | 5384 |
| Lead Tolerance | GATGCATTGGCTAAAGTTATGAATCACCGCCAAGACGAGTTTGTG | 5385 |
| cyclic nucleotide- | AGGTTTCAGGATTGGTAATCAGAGAGAAGCTCTGAGGGAAATTTT | |
| gated calmodulin- | CATGCTAAAGGTGGAGTCCACCGAAGTAAAG | |
| binding ion channel | CTTTACTTCGGTGGACTCCACCTTTAGCATGAAAATTTCCCTCAGA | 5386 |
| (CBP4) | GCTTCTCTCTGATTACCAATCCTGAAACCTCACAAACTCGTCTTGG | |
| Nicotiana Tabacum | CGGTGATTCATAACTTTAGCCAATGCATC | |
| Lys15Term | AGGATTGGTAATCAGAG | 5387 |
| AAA-TAA | CTCTGATTACCAATCCT | 5388 |
| Lead Tolerance | CTTGAAGAATTGATCTACCACTCTTAGCTGCTAACTGTTCGCCTGG | 5389 |
| calmodulin binding | TGGAGATAATGATGTAAAGAGAGGACAGATATGTTAGATTTCAGG | |
| transport protein | ACTGCAAATCAGAGCAATCTGTTATCTCAG | |
| Hordeum vulgare | CTGAGATAACAGATTGCTCTGATTTGCAGTCCTGAAATCTAACATA | 5390 |
| Glu2Term | TCTGTCCTCTCTTTACATCATTATCTCCACCAGGCGAACAGTTAGC | |
| GAA-TAA | AGCTAAGAGTGGTAGATCAATTCTTCAAG | |
| | TAATGATGTAAAGAGAG | 5391 |
| | CTCTCTTTACATCATTA | 5392 |
| Lead Tolerance | GAAGAATTGATCTACCACTCTTAGCTGCTAACTGTTCGCCTGGTG | 5393 |
| calmodulin binding | GAGATAATGATGGAATGAGAGGACAGATATGTTAGATTTCAGGAC | |
| transport protein | TGCAAATCAGAGCAATCTGTTATCTCAGAGA | |
| Hordeum vulgare | TCTCTGAGATAACAGATTGCTCTGATTTGCAGTCCTGAAATCTAAC | 5394 |
| Arg3Term | ATATCTGTCCTCTCATTCCATCATTATCTCCACCAGGCGAACAGTT | |
| AGA-TGA | AGCAGCTAAGAGTGGTAGATCAATTCTTC | |
| | TGATGGAATGAGAGGAC | 5395 |
| | GTCCTCTCATTCCATCA | 5396 |
| Lead Tolerance | GAATTGATCTACCACTCTTAGCTGCTAACTGTTCGCCTGGTGGAG | 5397 |
| calmodulin binding | ATAATGATGGAAAGATAGGACAGATATGTTAGATTTCAGGACTGC | |
| transport protein | AAATCAGAGCAATCTGTTATCTCAGAGAACG | |
| Hordeum vulgare | CGTTCTCTGAGATAACAGATTGCTCTGATTTGCAGTCCTGAAATCT | 5398 |
| Glu4Term | AACATATCTGTCCTATCTTTCCATCATTATCTCCACCAGGCGAACA | |
| GAG-TAG | GTTAGCAGCTAAGAGTGGTAGATCAATTC | |
| | TGGAAAGATAGGACAGA | 5399 |
| | TCTGTCCTATCTTTCCA | 5400 |
| Lead Tolerance | ATCTACCACTCTTAGCTGCTAACTGTTCGCCTGGTGGAGATAATG | 5401 |
| calmodulin binding | ATGGAAAGAGAGGACTGATATGTTAGATTTCAGGACTGCAAATCA | |
| transport protein | GAGCAATCTGTTATCTCAGAGAACGCAGTTT | |
| Hordeum vulgare | AAACTGCGTTCTCTGAGATAACAGATTGCTCTGATTTGCAGTCCTG | 5402 |
| Arg6Term | AAATCTAACATATCAGTCCTCTCTTTCCATCATTATCTCCACCAGG | |
| AGA-TGA | CGAACAGTTAGCAGCTAAGAGTGGTAGAT | |
| | GAGAGGACTGATATGTT | 5403 |
| | AACATATCAGTCCTCTC | 5404 |
| Lead Tolerance | CCACTCTTAGCTGCTAACTGTTCGCCTGGTGGAGATAATGATGGA | 5405 |
| calmodulin binding | AAGAGAGGACAGATAGGTTAGATTTCAGGACTGCAAATCAGAGCA | |
| transport protein | ATCTGTTATCTCAGAGAACGCAGTTTCACCA | |
| Hordeum vulgare | TGGTGAAACTGCGTTCTCTGAGATAACAGATTGCTCTGATTTGCA | 5406 |
| Tyr7Term | GTCCTGAAATCTAACCTATCTGTCCTCTCTTTCCATCATTATCTCCA | |
| TAT-TAG | CCAGGCGAACAGTTAGCAGCTAAGAGTGG | |
| | GACAGATAGGTTAGATT | 5407 |
| | AATCTAACCTATCTGTC | 5408 |
| 2,4-DB resistance | ATCCTTCTCTGAGAAAAAACAACAGATCCGAATTTTATCTTTAATCA | 5409 |
| 3-ketoacyl-CoA | GCCGGAAAAAATGTAGAAAGCGATCGAGAGACAACGCGTTCTTCT | |
| thiolase | TGAGCATCTCCGACCTTCTTCTTCTTCTT | |
| Arabidopsis thaliana | AAGAAGAAGAAGAAGGTCGGAGATGCTCAAGAAGAACGCGTTGT | 5410 |
| Glu2Term | CTCTCGATCGCTTTCTACATTTTTTCCGGCTGATTAAAGATAAAATT | |
| GAG-TAG | CGGATCTGTTGTTTTTTCTCAGAGAAGGAT | |
| | AAAAAATGTAGAAAGCG | 5411 |
| | CGCTTTCTACATTTTTT | 5412 |
| 2,4-DB resistance | CTTCTCTGAGAAAAAACAACAGATCCGAATTTTATCTTTAATCAGC | 5413 |
| 3-ketoacyl-CoA | CGGAAAAAATGGAGTAAGCGATCGAGAGACAACGCGTTCTTCTTG | |
| thiolase | AGCATCTCCGACCTTCTTCTTCTTCTTCGC | |
| Arabidopsis thaliana | GCGAAGAAGAAGAAGAAGGTCGGAGATGCTCAAGAAGAACGCGT | 5414 |
| Lys3Term | TGTCTCTCGATCGCTTACTCCATTTTTTCCGGCTGATTAAAGATAA | |
| AAA-TAA | AATTCGGATCTGTTGTTTTTTCTCAGAGAAG | |
| | AAATGGAGTAAGCGATC | 5415 |
| | GATCGCTTACTCCATTT | 5416 |
| 2,4-DB resistance | GAAAAAACAACAGATCCGAATTTTATCTTTAATCAGCCGGAAAAAA | 5417 |
| 3-ketoacyl-CoA | TGGAGAAAGCGATCTAGAGACAACGCGTTCTTCTTGAGCATCTCC | |
| thiolase | GACCTTCTTCTTCTTCTTCGCACAATTACG | |
| Arabidopsis thaliana | CGTAATTGTGCGAAGAAGAAGAAGAAGGTCGGAGATGCTCAAGA | 5418 |
| Glu6Term | AGAACGCGTTGTCTCTAGATCGCTTTCTCCATTTTTTCCGGCTGAT | |
| GAG-TAG | TAAAGATAAAATTCGGATCTGTTGTTTTTTC | |
| | AAGCGATCTAGAGACAA | 5419 |
| | TTGTCTCTAGATCGCTT | 5420 |
| 2,4-DB resistance | AAAACAACAGATCCGAATTTTATCTTTAATCAGCCGGAAAAAATGG | 5421 |
| 3-ketoacyl-CoA | AGAAAGCGATCGAGTGACAACGCGTTCTTCTTGAGCATCTCCGAC | |
| thiolase | CTTCTTCTTCTTCTTCGCACAATTACGAGG | |
| Arabidopsis thaliana | CCTCGTAATTGTGCGAAGAAGAAGAAGAAGGTCGGAGATGCTCAA | 5422 |
| Arg7Term | GAAGAACGCGTTGTCACTCGATCGCTTTCTCCATTTTTTCCGGCT | |
| AGA-TGA | GATTAAAGATAAAATTCGGATCTGTTGTTTT | |
| | CGATCGAGTGACAACGC | 5423 |
| | GCGTTGTCACTCGATCG | 5424 |
| 2,4-DB resistance | ACAACAGATCCGAATTTTATCTTTAATCAGCCGGAAAAAATGGAGA | 5425 |
| 3-ketoacyl-CoA | AAGCGATCGAGAGATAACGCGTTCTTCTTGAGCATCTCCGACCTT | |
| thiolase | CTTCTTCTTCTTCGCACAATTACGAGGCTT | |
| Arabidopsis thaliana | AAGCCTCGTAATTGTGCGAAGAAGAAGAAGAAGGTCGGAGATGC | 5426 |
| Gln8Term | TCAAGAAGAACGCGTTATCTCTCGATCGCTTTCTCCATTTTTTCCG | |
| CAA-TAA | GCTGATTAAAGATAAAATTCGGATCTGTTGT | |
| | TCGAGAGATAACGCGTT | 5427 |
| | AACGCGTTATCTCTCGA | 5428 |
| 2,4-DB resistance | GAGAGACAAAGAGTTCTTCTTGAACATCTCCGTCCTTCTTCTTCTT | 5429 |
| glyoxysomal beta- | CCTCTCACAGCTTTTAAGGCTCTCTCTCTGCTTCAGCTTGCTTGGC | |
| ketoacyol-thiolase | TGGGGACAGTGCTGCGTATCAGAGGACCT | |
| precursor | AGGTCCTCTGATACGCAGCACTGTCCCCAGCCAAGCAAGCTGAA | 5430 |
| Brassica napus | GCAGAGAGAGAGCCTTAAAAGCTGTGAGAGGAAGAAGAAGAAGG | |
| Glu26Term | ACGGAGATGTTCAAGAAGAACTCTTTGTCTCTC | |
| GAA-TAA | ACAGCTTTTAAGGCTCT | 5431 |
| | AGAGCCTTAAAAGCTGT | 5432 |
| 2,4-DB resistance | TTGAACATCTCCGTCCTTCTTCTTCTTCCTCTCACAGCTTTGAAGG | 5433 |
| glyoxysomal beta- | CTCTCTCTCTGCTTGAGCTTGCTTGGCTGGGGACAGTGCTGCGTA | |
| ketoacyol-thiolase | TCAGAGGACCTCTCTCTATGGAGATGATGT | |
| precursor | ACATCATCTCCATAGAGAGAGGTCCTCTGATACGCAGCACTGTCC | 5434 |
| Brassica napus | CCAGCCAAGCAAGCTCAAGCAGAGAGAGAGCCTTCAAAGCTGTG | |
| Ser32Term | AGAGGAAGAAGAAGAAGGACGGAGATGTTCAA | |
| TCA-TGA | CTCTGCTTGAGCTTGCT | 5435 |
| | AGCAAGCTCAAGCAGAG | 5436 |
| 2,4-DB resistance | TCTCCGTCCTTCTTCTTCTTCCTCTCACAGCTTTGAAGGCTCTCTC | 5437 |
| glyoxysomal beta- | TCTGCTTCAGCTTGATTGGCTGGGGACAGTGCTGCGTATCAGAG | |
| ketoacyol-thiolase | GACCTCTCTCTATGGAGATGATGTAGTCATT | |
| precursor | AATGACTACATCATCTCCATAGAGAGAGGTCCTCTGATACGCAGC | 5438 |
| Brassica napus | ACTGTCCCCAGCCAATCAAGCTGAAGCAGAGAGAGAGCCTTCAAA | |
| Cys34Term | GCTGTGAGAGGAAGAAGAAGAAGGACGGAGA | |
| TGC-TGA | TCAGCTTGATTGGCTGG | 5439 |
| | CCAGCCAATCAAGCTGA | 5440 |
| 2,4-DB resistance | TCCGTCCTTCTTCTTCTTCCTCTCACAGCTTTGAAGGCTCTCTCTC | 5441 |
| glyoxysomal beta- | TGCTTCAGCTTGCTAGGCTGGGGACAGTGCTGCGTATCAGAGGA | |
| ketoacyol-thiolase | CCTCTCTCTATGGAGATGATGTAGTCATTGT | |
| precursor | ACAATGACTACATCATCTCCATAGAGAGAGGTCCTCTGATACGCA | 5442 |
| Brassica napus | GCACTGTCCCCAGCCTAGCAAGCTGAAGCAGAGAGAGAGCCTTC | |
| Leu35Term | AAAGCTGTGAGAGGAAGAAGAAGAAGGACGGA | |
| TTG-TAG | AGCTTGCTAGGCTGGGG | 5443 |
| | CCCCAGCCTAGCAAGCT | 5444 |
| 2,4-DB resistance | TCACAGCTTTGAAGGCTCTCTCTCTGCTTCAGCTTGCTTGGCTGG | 5445 |
| glyoxysomal beta- | GGACAGTGCTGCGTAGCAGAGGACCTCTCTCTATGGAGATGATGT | |
| ketoacyol-thiolase | AGTCATTGTTGCGGCACATAGGACTGCACTA | |
| precursor | TAGTGCAGTCCTATGTGCCGCAACAATGACTACATCATCTCCATA | 5446 |
| Brassica napus | GAGAGAGGTCCTCTGCTACGCAGCACTGTCCCCAGCCAAGCAAG | |
| Tyr42Term | CTGAAGCAGAGAGAGAGCCTTCAAAGCTGTGA | |
| TAT-TAG | GCTGCGTAGCAGAGGAC | 5447 |
| | GTCCTCTGCTACGCAGC | 5448 |
| 2,4-DB resistance | CAACAGACAGCAAGTGTTGCTCCAGCATCTCCGCCCTTCTAATTC | 5449 |
| 3-ketoacyl-CoA | TTCTTCTCACAATTAGGAGTCCGCTCTTGCCGCATCAGTATGTGCT | |
| thiolase B | GCAGGGGATAGCGCCGCATATCATAGGGCT | |
| Mangifera indica | AGCCCTATGATATGCGGCGCTATCCCCTGCAGCACATACTGATGC | 5450 |
| Tyr25Term | GGCAAGAGCGGACTCCTAATTGTGAGAAGAAGAATTAGAAGGGC | |
| TAC-TAG | GGAGATGCTGGAGCAACACTTGCTGTCTGTTG | |
| | CACAATTAGGAGTCCGC | 5451 |
| | GCGGACTCCTAATTGTG | 5452 |
| 2,4-DB resistance | AACAGACAGCAAGTGTTGCTCCAGCATCTCCGCCCTTCTAATTCTT | 5453 |
| 3-ketoacyol-CoA | CTTCTCACAATTACTAGTCCGCTCTTGCCGCATCAGTATGTGCTGC | |
| thiolase B | AGGGGATAGCGCCGCATATCATAGGGCTT | |
| Magnifera indica | AAGCCCTATGATATGCGGCGCTATCCCCTGCAGCACATACTGATG | 5454 |
| Glu26Term | CGGCAAGAGCGGACTAGTAATTGTGAGAAGAAGAATTAGAAGGG | |
| GAG-TAG | CGGAGATGCTGGAGCAACACTTGCTGTCTGTT | |
| | ACAATTACTAGTCCGCT | 5455 |
| | AGCGGACTAGTAATTGT | 5456 |
| 2,4-DB resistance | TCCAGCATCTCCGCCCTTCTAATTCTTCTTCTCACAATTACGAGTC | 5457 |
| 3-ketoacy\ol-CoA | CGCTCTTGCCGCATGAGTATGTGCTGCAGGGGATAGCGCCGCAT | |
| thiolase B | ATCATAGGGCTTCTGTTTATGGAGACGATGT | |
| Mangifera indica | ACATCGTCTCCATAAACAGAAGCCCTATGATATGCGGCGCTATCC | 5458 |
| Ser32Term | CCTGCAGCACATACTCATGCGGCAAGAGCGGACTCGTAATTGTGA | |
| TCA-TGA | GAAGAAGAATTAGAAGGGCGGAGATGCTGGA | |
| | TGCCGCATGAGTATGTG | 5459 |
| | CACATACTCATGCGGCA | 5460 |
| 2,4-DB resistance | TCTCCGCCCTTCTAATTCTTCTTCTCACAATTACGAGTCCGCTCTT | 5461 |
| 3-ketoacyl-CoA | GCCGCATCAGTATGAGCTGCAGGGGATAGCGCCGCATATCATAG | |
| thiolase B | GGCTTCTGTTTATGGAGACGATGTGGTGATT | |
| Mangifera indica | AATCACCACATCGTCTCCATAAACAGAAGCCCTATGATATGCGGC | 5462 |
| Cys34Term | GCTATCCCCTGCAGCTCATACTGATGCGGCAAGAGCGGACTCGT | |
| TGT-TGA | AATTGTGAGAAGAAGAATTAGAAGGGCGGAGA | |
| | TCAGTATGAGCTGCAGG | 5463 |
| | CCTGCAGCTCATACTGA | 5464 |
| 2,4-DB resistance | TCACAATTACGAGTCCGCTCTTGCCGCATCAGTATGTGCTGCAGG | 5465 |
| 3-ketoacyl-CoA | GGATAGCGCCGCATAGCATAGGGCTTCTGTTTATGGAGACGATGT | |
| thiolase B | GGTGATTGTGGCAGCTCATCGTACTGCACTT | |
| Mangifera indica | AAGTGCAGTACGATGAGCTGCCACAATCACCACATCGTCTCCATA | 5466 |
| Tyr42Term | AACAGAAGCCCTATGCTATGCGGCGCTATCCCCTGCAGCACATAC | |
| TAT-TAG | TGATGCGGCAAGAGCGGACTCGTAATTGTGA | |
| | GCCGCATAGCATAGGGC | 5467 |
| | GCCCTATGCTATGCGGC | 5468 |
| 2,4-DB resistance | GAAGGCGATCAACAGGCAGAGCATTTTGCTACATCATCTCCGGCC | 5469 |
| 3-ketoacyl-CoA | TTCTTCTTCCGCTTAGACAAATGAATCTTCGCTCTCTGCATCGGTT | |
| thiolase | TGTGCAGCTGGGGATAGTGCTTCGTATCAA | |
| Cucumis sativus | TTGATACGAAGCACTATCCCCAGCTGCACAAACCGATGCAGAGAG | 5470 |
| Tyr22Term | CGAAGATTCATTTGTCTAAGCGGAAGAAGAAGGCCGGAGATGATG | |
| TAC-TAG | TAGCAAAATGCTCTGCCTGTTGATCGCCTTC | |
| | TCCGCTTAGACAAATGA | 5471 |
| | TCATTTGTCTAAGCGGA | 5472 |
| 2,4-DB resistance | ATCAACAGGCAGAGCATTTTGCTACATCATCTCCGGCCTTCTTCTT | 5473 |
| 3-ketoacyl-CoA | CCGCTTACACAAATTAATCTTCGCTCTCTGCATCGGTTTGTGCAGC | |
| thiolase | TGGGGATAGTGCTTCGTATCAAAGGACAT | |
| Cucumis sativus | ATGTCCTTTGATACGAAGCACTATCCCCAGCTGCACAAACCGATG | 5474 |
| Glu25Term | CAGAGAGCGAAGATTAATTTGTGTAAGCGGAAGAAGAAGGCCGG | |
| GAA-TAA | AGATGATGTAGCAAAATGCTCTGCCTGTTGAT | |
| | ACACAAATTAATCTTCG | 5475 |
| | CGAAGATTAATTTGTGT | 5476 |
| 2,4-DB resistance | GGCAGAGCATTTTGCTACATCATCTCCGGCCTTCTTCTTCCGCTTA | 5477 |
| 3-ketoacyl-CoA | CACAAATGAATCTTAGCTCTCTGCATCGGTTTGTGCAGCTGGGGA | |
| thiolase | TAGTGCTTCGTATCAAAGGACATCGGTGTT | |
| Cucumis sativus | AACACCGATGTCCTTTGATACGAAGCACTATCCCCAGCTGCACAA | 5478 |
| Ser27Term | ACCGATGCAGAGAGCTAAGATTCATTTGTGTAAGCGGAAGAAGAA | |
| TCG-TAG | GGCCGGAGATGATGTAGCAAAATGCTCTGCC | |
| | TGAATCTTAGCTCTCTG | 5479 |
| | CAGAGAGCTAAGATTCA | 5480 |
| 2,4-DB resistance | TGCTACATCATCTCCGGCCTTCTTCTTCCGCTTACACAAATGAATC | 5481 |
| 3-ketoacyl-CoA | TTCGCTCTCTGCATAGGTTTGTGCAGCTGGGGATAGTGCTTCGTA | |
| thiolase | TCAAAGGACATCGGTGTTTGGAGATGATGT | |
| Cucumis sativus | ACATCATCTCCAAACACCGATGTCCTTTGATACGAAGCACTATCCC | 5482 |
| Ser31Term | CAGCTGCACAAACCTATGCAGAGAGCGAAGATTCATTTGTGTAAG | |
| TCG-TAG | CGGAAGAAGAAGGCCGGAGATGATGTAGCA | |
| | CTCTGCATAGGTTTGTG | 5483 |
| | CACAAACCTATGCAGAG | 5484 |
| 2,4-DB resistance | TCATCTCCGGCCTTCTTCTTCCGCTTACACAAATGAATCTTCGCTC | 5485 |
| 3-ketoacyl-CoA | TCTGCATCGGTTTGAGCAGCTGGGGATAGTGCTTCGTATCAAAGG | |
| thiolase | ACATCGGTGTTTGGAGATGATGTCGTGATT | |
| Cucumis sativus | AATCACGACATCATCTCCAAACACCGATGTCCTTTGATACGAAGCA | 5486 |
| Cys33Term | CTATCCCCAGCTGCTCAAACCGATGCAGAGAGCGAAGATTCATTT | |
| TGT-TGA | GTGTAAGCGGAAGAAGAAGGCCGGAGATGA | |
| | TCGGTTTGAGCAGCTGG | 5487 |
| | CCAGCTGCTCAAACCGA | 5488 |
| 2,4-DB resistance | GAAGGCAATCAACAGGCAGAGCATTCTGCTACATCATCTCCGGCC | 5489 |
| 3-ketoacyl-CoA | TTCATCTTCGGCTTAGAGCCATGAATCTTCGCTCTCTGCATCGGTT | |
| thiolase | TGTGCAGCTGGGGATAGTGCGTCGTATCAA | |
| Cucurbita sp. | TTGATACGACGCACTATCCCCAGCTGCACAAACCGATGCAGAGAG | 5490 |
| Tyr22Term | CGAAGATTCATGGCTCTAAGCCGAAGATGAAGGCCGGAGATGAT | |
| TAT-TAG | GTAGCAGAATGCTCTGCCTGTTGATTGCCTTC | |
| | TCGGCTTAGAGCCATGA | 5491 |
| | TCATGGCTCTAAGCCGA | 5492 |
| 2,4-DB resistance | ATCAACAGGCAGAGCATTCTGCTACATCATCTCCGGCCTTCATCTT | 5493 |
| 3-ketoacyl-CoA | CGGCTTATAGCCATTAATCTTCGCTCTCTGCATCGGTTTGTGCAGC | |
| thiolase | TGGGGATAGTGCGTCGTATCAAAGAACGT | |
| Cucurbita sp. | ACGTTCTTTGATACGACGCACTATCCCCAGCTGCACAAACCGATG | 5494 |
| Glu25Term | CAGAGAGCGAAGATTAATGGCTATAAGCCGAAGATGAAGGCCGG | |
| GAA-TAA | AGATGATGTAGCAGAATGCTCTGCCTGTTGAT | |
| | ATAGCCATTAATCTTCG | 5495 |
| | CGAAGATTAATGGCTAT | 5496 |
| 2,4-DB resistance | GGCAGAGCATTCTGCTACATCATCTCCGGCCTTCATCTTCGGCTT | 5497 |
| 3-ketoacyl-CoA | ATAGCCATGAATCTTAGCTCTCTGCATCGGTTTGTGCAGCTGGGG | |
| thiolase | ATAGTGCGTCGTATCAAAGAACGTCGGTGTT | |
| Cucurbita sp. | AACACCGACGTTCTTTGATACGACGCACTATCCCCAGCTGCACAA | 5498 |
| Ser21Term | ACCGATGCAGAGAGCTAAGATTCATGGCTATAAGCCGAAGATGAA | |
| TCG-TAG | GGCCGGAGATGATGTAGCAGAATGCTCTGCC | |
| | TGAATCTTAGCTCTCTG | 5499 |
| | CAGAGAGCTAAGATTCA | 5500 |
| 2,4-DB resistance | TGCTACATCATCTCCGGCCTTCATCTTCGGCTTATAGCCATGAATC | 5501 |
| 3-ketoacyl-CoA | TTCGCTCTCTGCATAGGTTTGTGCAGCTGGGGATAGTGCGTCGTA | |
| thiolase | TCAAAGAACGTCGGTGTTTGGAGATGATGT | |
| Cucurbita sp. | ACATCATCTCCAAACACCGACGTTCTTTGATACGACGCACTATCCC | 5502 |
| Ser31Term | CAGCTGCACAAACCTATGCAGAGAGCGAAGATTCATGGCTATAAG | |
| TCG-TAG | CCGAAGATGAAGGCCGGAGATGATGTAGCA | |
| | CTCTGCATAGGTTTGTG | 5503 |
| | CACAAACCTATGCAGAG | 5504 |
| 2,4-DB resistance | TCATCTCCGGCCTTCATCTTCGGCTTATAGCCATGAATCTTCGCTC | 5505 |
| 3-ketoacyl-CoA | TCTGCATCGGTTTGAGCAGCTGGGGATAGTGCGTCGTATCAAAGA | |
| thiolase | ACGTCGGTGTTTGGAGATGATGTCGTGATA | |
| Cucurbita sp. | TATCACGACATCATCTCCAAACACCGACGTTCTTTGATACGACGCA | 5506 |
| Cys33Term | CTATCCCCAGCTGCTCAAACCGATGCAGAGAGCGAAGATTCATGG | |
| TGT-TGA | CTATAAGCCGAAGATGAAGGCCGGAGATGA | |
| | TCGGTTTGAGCAGCTGG | 5507 |
| | CCAGCTGCTCAAACCGA | 5508 |
| 2,4 DB resistance | TCATAGTCTCTTTTGCCGCTTGGATTCTTCCAAGGTTAGTGAGCTG | 5509 |
| Pex14 | CTATGGCAACTCATTAGCAAACGCAACCTCCTTCCGATTTTCCCGC | |
| Arabidopsis thaliana | TCTTGCCGATGAAAATTCCCAGATTCCAG | |
| Gln5Term | CTGGAATCTGGGAATTTTCATCGGCAAGAGCGGGAAAATCGGAA | 5510 |
| CAG-TAG | GGAGGTTGCGTTTGCTAATGAGTTGCCATAGCAGCTCACTAACCT | |
| | TGGAAGAATCCAAGCGGCAAAAGAGACTATGA | |
| | CAACTCATTAGCAAACG | 5511 |
| | CGTTTGCTAATGAGTTG | 5512 |
| 2,4 DB resistance | TAGTCTCTTTTGCCGCTTGGATTCTTCCAAGGTTAGTGAGCTGCTA | 5513 |
| Pex14 | TGGCAACTCATCAGTAAACGCAACCTCCTTCCGATTTTCCCGCTCT | |
| Arabidopsis thaliana | TGCCGATGAAAATTCCCAGATTCCAGGTT | |
| Gln6Term | AACCTGGAATCTGGGAATTTTCATCGGCAAGAGCGGGAAAATCGG | 5514 |
| CAA-TAA | AAGGAGGTTGCGTTTACTGATGAGTTGCCATAGCAGCTCACTAAC | |
| | CTTGGAAGAATCCAAGCGGCAAAAGAGACTA | |
| | CTCATCAGTAAACGCAA | 5515 |
| | TTGCGTTTACTGATGAG | 5516 |
| 2,4 DB resistance | CTTTTGCCGCTTGGATTCTTCCAAGGTTAGTGAGCTGCTATGGCA | 5517 |
| Pex14 | ACTCATCAGCAAACGTAACCTCCTTCCGATTTTCCCGCTCTTGCCG | |
| Arabidopsis thaliana | ATGAAAATTCCCAGATTCCAGGTTCAATTT | |
| Gln8Term | AAATTGAACCTGGAATCTGGGAATTTTCATCGGCAAGAGCGGGAA | 5518 |
| CAA-TAA | AATCGGAAGGAGGTTACGTTTGCTGATGAGTTGCCATAGCAGCTC | |
| | ACTAACCTTGGAAGAATCCAAGCGGCAAAAG | |
| | AGCAAACGTAACCTCCT | 5519 |
| | AGGAGGTTACGTTTGCT | 5520 |
| 2,4 DB resistance | GCTGCTATGGCAACTCATCAGCAAACGCAACCTCCTTCCGATTTT | 5521 |
| Pex14 | CCCGCTCTTGCCGATTAAAATTCCCAGATTCCAGGTTCAATTTACA | |
| Arabidopsis thaliana | CCTTCTAATCATTATTTCTTAATTTTTCTT | |
| Glu19Term | AAGAAAAATTAAGAAATAATGATTAGAAGGTGTAAATTGAACCTGG | 5522 |
| GAA-TAA | AATCTGGGAATTTTAATCGGCAAGAGCGGGAAAATCGGAAGGAG | |
| | GTTGCGTTTGCTGATGAGTTGCCATAGCAGC | |
| | TTGCCGATTAAAATTCC | 5523 |
| | GGAATTTTAATCGGCAA | 5524 |
| 2,4 DB resistance | GCAACTCATCAGCAAACGCAACCTCCTTCCGATTTTCCCGCTCTT | 5525 |
| Pex14 | GCCGATGAAAATTCCTAGATTCCAGGTTCAATTTACACCTTCTAAT | |
| Arabidopsis thaliana | CATTATTTCTTAATTTTTCTTTGGTGGATT | |
| Gln22Term | AATCCACCAAAGAAAAATTAAGAAATAATGATTAGAAGGTGTAAAT | 5526 |
| CAG-TAG | TGAACCTGGAATCTAGGAATTTTCATCGGCAAGAGCGGGAAAATC | |
| | GGAAGGAGGTTGCGTTTGCTGATGAGTTGC | |
| | AAAATTCCTAGATTCCA | 5527 |
| | TGGAATCTAGGAATTTT | 5528 |

### EXAMPLE 29

### Production of Albino Mutants for the Analysis of Photosynthetic Processes

Plant productivity is limited by resources available and the ability of plants to harness these resources. The conversion of light to chemical energy, which is then used to synthesize carbohydrates, fatty acids, sugars, amino acids and other compounds, requires a complex system which combines the light harvesting apparatus of pigments and proteins. The value of light energy to the plant can only be realized when it is efficiently converted into chemical energy by photosynthesis and fed into various biochemical processes. Significant effort has therefore been directed at studying photosynthetic processes in plants in order to improve productivity and/or the efficiency of photosynthesis. The analysis of the photosynthetic process is substantially aided by the ability to produce albino plants.

The attached table discloses exemplary oligonucleotide base sequences which can be used to generate site-specific mutations in genes involved in starch metabolism.

TABLE 36
Oligonucleotides to produce albino plants

| Phenotype, Gene, | | |
|---|---|---|
| Plant & Targeted | | SEQ ID |
| Alteration | Altering Oligos | NO: |
| White leaves | TTCTTTCCTGTGAAATTATCTGCTCAAATCTTTGGTTCCTGACGGAG | 5529 |
| Immutans | ATGGCGGCGATTTGAGGCATCTCCTCTGGTACGTTGACGATTTCA | |
| Arabidopsis thaliana | CGGCCTTTGGTTACTCTTCGACGCTCTAG | |
| Ser5Term | CTAGAGCGTCGAAGAGTAACCAAAGGCCGTGAAATCGTCAACGTA | 5530 |
| TCA-TGA | CCAGAGGAGATGCCTCAAATCGCCGCCATCTCCGTCAGGAACCAA | |
| | AGATTTGAGCAGATAATTTCACAGGAAAGAA | |
| | GGCGATTTGAGGCATCT | 5531 |
| | AGATGCCTCAAATCGCC | 5532 |
| White leaves | GCTCAAATCTTTGGTTCCTGACGGAGATGGCGGCGATTTCAGGCA | 5533 |
| Immutans | TCTCCTCTGGTACGTAGACGATTTCACGGCCTTTGGTTACTCTTCG | |
| Arabidopsis thaliana | ACGCTCTAGAGCCGCCGTTTCGTACAGCTC | |
| Leu12Term | GAGCTGTACGAAACGGCGGCTCTAGAGCGTCGAAGAGTTAACCAAA | 5534 |
| TTG-TAG | GGCCGTGAAATCGTCTACGTACCAGAGGAGATGCCTGAAATCGCC | |
| | GCCATCTCCGTCAGGAACCAAAGATTTGAGC | |
| | TGGTACGTAGACGATTT | 5535 |
| | AAATCGTCTACGTACCA | 5536 |
| White leaves | TTTGGTTCCTGACGGAGATGGCGGCGATTTCAGGCATCTCCTCTG | 5537 |
| Immutans | GTACGTTGACGATTTGACGGCCTTTGGTTACTCTTCGACGCTCTAG | |
| Arabidopsis thaliana | AGCCGCCGTTTCGTACAGCTCCTCTCACCG | |
| Ser15Term | CGGTGAGAGGAGCTGTACGAAACGGCGGCTCTAGAGCGTCGAAG | 5538 |
| TCA-TGA | AGTAACCAAAGGCCGTCAAATCGTCAACGTACCAGAGGAGATGCC | |
| | TGAAATCGCCGCCATCTCCGTCAGGAACCAAA | |
| | GACGATTTGACGGCCTT | 5539 |
| | AAGGCCGTCAAATCGTC | 5540 |
| White leaves | GCGGCGATTTCAGGCATCTCCTCTGGTACGTTGACGATTTCACGG | 5541 |
| Immutans | CCTTTGGTTACTCTTTGACGCTCTAGAGCCGCCGTTTCGTACAGCT | |
| Arabidopsis thaliana | CCTCTCACCGATTGCTTCATCATCTTCCTC | |
| Arg22Term | GAGGAAGATGATGAAGCAATCGGTGAGAGGAGCTGTACGAAACG | 5542 |
| CGA-TGA | GCGGCTCTAGAGCGTCAAAGAGTAACCAAAGGCCGTGAAATCGTC | |
| | AACGTACCAGAGGAGATGCCTGAAATCGCCGC | |
| | TTACTCTTTGACGCTCT | 5543 |
| | AGAGCGTCAAAGAGTAA | 5544 |
| White leaves | TCAGGCATCTCCTCTGGTACGTTGACGATTTCACGGCCTTTGGTTA | 5545 |
| Immutans | CTCTTCGACGCTCTTGAGCCGCCGTTTCGTACAGCTCCTCTCACC | |
| Arabidopsis thaliana | GATTGCTTCATCATCTTCCTCTCTCTTCTC | |
| Arg25Term | GAGAAGAGAGAGGAAGATGATGAAGCAATCGGTGAGAGGAGCTG | 5546 |
| AGA-TGA | TACGAAACGGCGGCTCAAGAGCGTCGAAGAGTAACCAAAGGCCG | |
| | TGAAATCGTCAACGTACCAGAGGAGATGCCTGA | |
| | GACGCTCTTGAGCCGCC | 5547 |
| | GGCGGCTCAAGAGCGTC | 5548 |
| White leaves | GATTCTTGTGGGAAGGAAGAAGGATCAAGAATGGCGATTTCGATT | 5549 |
| Immutans | TCTGCTATGAGTTTTTGAACCTCAGTTTCTTCATATTCTTGTTTTAG | |
| Lycopersicon | AGCTAGGAGTTTTGAGAAGTCATCAGTTT | |
| esculentum | AAACTGATGACTTCTCAAAACTCCTAGCTCTAAAACAAGAATATGA | 5550 |
| Gly11Term | AGAAACTGAGGTTCAAAAACTCATAGCAGAAATCGAAATCGCCATT | |
| GGA-TGA | CTTGATCCTTCTTCCTTCCCACAAGAATC | |
| | TGAGTTTTTGAACCTCA | 5551 |
| | TGAGGTTCAAAAACTCA | 5552 |
| White leaves | GTGGGAAGGAAGAAGGATCAAGAATGGCGATTTCGATTTCTGCTA | 5553 |
| Immutans | TGAGTTTTGGAACCTGAGTTTCTTCATATTCTTGTTTTAGAGCTAGG | |
| Lycopersicon | AGTTTTGAGAAGTCATCAGTTTTATGCAA | |
| esculentum | TTGCATAAAACTGATGACTTCTCAAAACTCCTAGCTCTAAAACAAG | 5554 |
| Ser13Term | AATATGAAGAAACTCAGGTTCCAAAACTCATAGCAGAAATCGAAAT | |
| TCA-TGA | CGCCATTCTTGATCCTTCTTCCTTCCCAC | |
| | TGGAACCTGAGTTTCTT | 5555 |
| | AAGAAACTCAGGTTCCA | 5556 |
| White leaves | AAGAAGGATCAAGAATGGCGATTTCGATTTCTGCTATGAGTTTTGG | 5557 |
| Immutans | AACCTCAGTTTCTTGATATTCTTGTTTTAGAGCTAGGAGTTTTGAGA | |
| Lycopersicon | AGTCATCAGTTTTATGCAATTCCCAGAA | |
| esculentum | TTCTGGGAATTGCATAAAACTGATGACTTCTCAAAACTCCTAGCTC | 5558 |
| Ser16Term | TAAAACAAGAATATCAAGAAACTGAGGTTCCAAAACTCATAGCAGA | |
| TCA-TGA | AATCGAAATCGCCATTCTTGATCCTTCTT | |
| | AGTTTCTTGATATTCTT | 5559 |
| | AAGAATATCAAGAAACT | 5560 |
| White leaves | AGGATCAAGAATGGCGATTTCGATTTCTGCTATGAGTTTTGGAACC | 5561 |
| Immutans | TCAGTTTCTTCATAGTCTTGTTTTAGAGCTAGGAGTTTTGAGAAGTC | |
| Lycopersicon | ATCAGTTTTATGCAATTCCCAGAACCCA | |
| esculentum | TGGGTTCTGGGAATTGCATAAAACTGATGACTTCTCAAAACTCCTA | 5562 |
| Tyr17Term | GCTCTAAAACAAGACTATGAAGAAACTGAGGTTCCAAAACTCATAG | |
| TAT-TAG | CAGAAATCGAAATCGCCATTCTTGATCCT | |
| | TCTTCATAGTCTTGTTT | 5563 |
| | AAACAAGACTATGAAGA | 5564 |
| White leaves | AAGAATGGCGATTTCGATTTCTGCTATGAGTTTTGGAACCTCAGTT | 5565 |
| Immutans | TCTTCATATTCTTGATTTAGAGCTAGGAGTTTTGAGAAGTCATCAGT | |
| Lycopersicon | TTTATGCAATTCCCAGAACCCATGTCGG | |
| esculentum | CCGACATGGGTTCTGGGAATTGCATAAAACTGATGACTTCTCAAAA | 5566 |
| Cys19Term | CTCCTAGCTCTAAATCAAGAATATGAAGAAACTGAGGTTCCAAAAC | |
| TGT-TGA | TCATAGCAGAAATCGAAATCGCCATTCTT | |
| | TATTCTTGATTTAGAGC | 5567 |
| | GCTCTAAATCAAGAATA | 5568 |
| White leaves | CGCGTCCGATAAAAAAATCAAGAATGGCGATTTCCATATCTGCTAT | 5569 |
| Immutans | GAGTTTTCGAACTTGAGTTTCTTCTTCATATTCAGCATTTTTGTGCA | |
| Capsicum annuum | ATTCCAAGAACCCATTTTGTTTGAATTC | |
| Ser13Term | GAATTCAAACAAAATGGGTTCTTGGAATTGCACAAAAATGCTGAAT | 5570 |
| TCA-TGA | ATGAAGAAGAAACTCAAGTTCGAAAACTCATAGCAGATATGGAAAT | |
| | CGCCATTCTTGATTTTTTTATCGGACGCG | |
| | TCGAACTTGAGTTTCTT | 5571 |
| | AAGAAACTCAAGTTCGA | 5572 |
| White leaves | AAAAATCAAGAATGGCGATTTCCATATCTGCTATGAGTTTTCGAAC | 5573 |
| Immutans | TTCAGTTTCTTCTTGATATTCAGCATTTTTGTGCAATTCCAAGAACC | |
| Capsicum annuum | CATTTTGTTTGAATTCTCTATTTTCACT | |
| Ser17Term | AGTGAAAATAGAGAATTCAAACAAAATGGGTTCTTGGAATTGCACA | 5574 |
| TCA-TGA | AAAATGCTGAATATCAAGAAGAAACTGAAGTTCGAAAACTCATAGC | |
| | AGATATGGAAATCGCCATTCTTGATTTTT | |
| | TTCTTCTTGATATTCAG | 5575 |
| | CTGAATATCAAGAAGAA | 5576 |
| White leaves | CAAGAATGGCGATTTCCATATCTGCTATGAGTTTTCGAACTTCAGT | 5577 |
| Immutans | TTCTTCTTCATATTGAGCATTTTTGTGCAATTCCAAGAACCCATTTT | |
| Capsicum annuum | GTTTGAATTCTCTATTTTCACTTAGGAA | |
| Ser19Term | TTCCTAAGTGAAAATAGAGAATTCAAACAAAATGGGTTCTTGGAAT | 5578 |
| TCA-TGA | TGCACAAAAATGCTCAATATGAAGAAGAAACTGAAGTTCGAAAACT | |
| | CATAGCAGATATGGAAATCGCCATTCTTG | |
| | TTCATATTGAGCATTTT | 5579 |
| | AAAATGCTCAATATGAA | 5580 |
| White leaves | CGATTTCCATATCTGCTATGAGTTTTCGAACTTCAGTTTCTTCTTCA | 5581 |
| Immutans | TATTCAGCATTTTAGTGCAATTCCAAGAACCCATTTTGTTTGAATTC | |
| Capsicum annuum | TCTATTTTCACTTAGGAATTCTCATAG | |
| Leu21Term | CTATGAGAATTCCTAAGTGAAAATAGAGAATTCAAACAAAATGGGT | 5582 |
| TTG-TAG | TCTTGGAATTGCACTAAAATGCTGAATATGAAGAAGAAACTGAAGT | |
| | TCGAAAACTCATAGCAGATATGGAAATCG | |
| | AGCATTTTAGTGCAATT | 5583 |
| | AATTGCACTAAAATGCT | 5584 |
| White leaves | TTCCATATCTGCTATGAGTTTTCGAACTTCAGTTTCTTCTTCATATT | 5585 |
| Immutans | CAGCATTTTTGTGAAATTCCAAGAACCCATTTTGTTTGAATTCTCTA | |
| Capsicum annuum | TTTTCACTTAGGAATTCTCATAGAACT | |
| Cys22Term | AGTTCTATGAGAATTCCTAAGTGAAAATAGAGAATTCAAACAAAAT | 5586 |
| TGC-TGA | GGGTTCTTGGAATTTCACAAAAATGCTGAATATGAAGAAGAAACTG | |
| | AAGTTCGAAAACTCATAGCAGATATGGAA | |
| | TTTTTGTGAAATTCCAA | 5587 |
| | TTGGAATTTCACAAAAA | 5588 |
| White leaves | TTCGGCACGAGGGAGAAGGAGCAGACCGAGGTGGCCGTCGAGG | 5589 |
| Immutans | AGTCCTTCCCCTTCAGGTAGACGGCTCCTCCTGACGAGCCACTGG | |
| Oryza sativa | TCACCGCCGAGGAGAGCTGGGTGGTTAAGCTCG | |
| Glu22Term | CGAGCTTAACCACCCAGCTCTCCTCGGCGGTGACCAGTGGCTCGT | 5590 |
| GAG-TAG | CAGGAGGAGCCGTCTACCTGAAGGGGAAGGACTCCTCGACGGCC | |
| | ACCTCGGTCTGCTCCTTCTCCCTCGTGCCGAA | |
| | CCTTCAGGTAGACGGCT | 5591 |
| | AGCCGTCTACCTGAAGG | 5592 |
| White leaves | GAGCAGACCGAGGTGGCCGTCGAGGAGTCCTTCCCCTTCAGGGA | 5593 |
| Immutans | GACGGCTCCTCCTGACTAGCCACTGGTCACCGCCGAGGAGAGCT | |
| Oryza sativa | GGGTGGTTAAGCTCGAGCAGTCCGTGAACATTT | |
| Glu28Term | AAATGTTCACGGACTGCTCGAGCTTAACCACCCAGCTCTCCTCGG | 5594 |
| GAG-TAG | CGGTGACCAGTGGCTAGTCAGGAGGAGCCGTCTCCCTGAAGGGG | |
| | AAGGACTCCTCGACGGCCACCTCGGTCTGCTC | |
| | CTCCTGACTAGCCACTG | 5595 |
| | CAGTGGCTAGTCAGGAG | 5596 |
| White leaves | GTCGAGGAGTCCTTCCCCTTCAGGGAGACGGCTCCTCCTGACGA | 5597 |
| Immutans | GCCACTGGTCACCGCCTAGGAGAGCTGGGTGGTTAAGCTCGAGC | |
| Oryza sativa | AGTCCGTGAACATTTTCCTCACGGAGTCAGTCA | |
| Glu34Term | TGACTGACTCCGTGAGGAAAATGTTCACGGACTGCTCGAGCTTAA | 5598 |
| GAG-TAG | CCACCCAGCTCTCCTAGGCGGTGACCAGTGGCTCGTCAGGAGGA | |
| | GCCGTCTCCCTGAAGGGGAAGGACTCCTCGAC | |
| | TCACCGCCTAGGAGAGC | 5599 |
| | GCTCTCCTAGGCGGTGA | 5600 |
| White leaves | GAGGAGTCCTTCCCCTTCAGGGAGACGGCTCCTCCTGACGAGCC | 5601 |
| Immutans | ACTGGTCACCGCCGAGTAGAGCTGGGTGGTTAAGCTCGAGCAGT | |
| Oryza sativa | CCGTGAACATTTTCCTCACGGAGTCAGTCATCA | |
| Glu35Term | TGATGACTGACTCCGTGAGGAAAATGTTCACGGACTGCTCGAGCT | 5602 |
| GAG-TAG | TAACCACCCAGCTCTACTCGGCGGTGACCAGTGGCTCGTCAGGA | |
| | GGAGCCGTCTCCCTGAAGGGGAAGGACTCCTC | |
| | CCGCCGAGTAGAGCTGG | 5603 |
| | CCAGCTCTACTCGGCGG | 5604 |
| White leaves | CTTCCCCTTCAGGGAGACGGCTCCTCCTGACGAGCCACTGGTCAC | 5605 |
| Immutans | CGCCGAGGAGAGCTGAGTGGTTAAGCTCGAGCAGTCCGTGAACA | |
| Oryza sativa | TTTTCCTCACGGAGTCAGTCATCACGATACTT | |
| Trp37Term | AAGTATCGTGATGACTGACTCCGTGAGGAAAATGTTCACGGACTG | 5606 |
| TGG-TGA | CTCGAGCTTAACCACTCAGCTCTCCTCGGCGGTGACCAGTGGCTC | |
| | GTCAGGAGGAGCCGTCTCCCTGAAGGGGAAG | |
| | GAGAGCTGAGTGGTTAA | 5607 |
| | TTAACCACTCAGCTCTC | 5608 |
| White leaves | TCCGGAGGAGGAAGGGGGATTCGACGAGGAGCTCACCCTCGCCG | 5609 |
| Immutans | GCGAGGACGGCGACTGAGTCGTCAGATTCGAGCAGTCCTTCAAC | |
| Triticum aestivum | GTATTCCTCACGGATACTGTCATCTTTATACTC | |
| Trp22Term | GAGTATAAAGATGACAGTATCCGTGAGGAATACGTTGAAGGACTG | 5610 |
| TGG-TGA | CTCGAATCTGACGACTCAGTCGCCGTCCTCGCCGGCGAGGGTGA | |
| | GCTCCTCGTCGAATCCCCCTTCCTCCTCCGGA | |
| | GGCGACTGAGTCGTCAG | 5611 |
| | CTGACGACTCAGTCGCC | 5612 |
| White leaves | GAGGAAGGGGGATTCGACGAGGAGCTCACCCTCGCCGGCGAGG | 5613 |
| Immutans | ACGGCGACTGGGTCGTCTGATTCGAGCAGTCCTTCAACGTATTCC | |
| Triticum aestivum | TCACGGATACTGTCATCTTTATACTCGATATTC | |
| Arg25Term | GAATATCGAGTATAAAGATGACAGTATCCGTGAGGAATACGTTGAA | 5614 |
| AGA-TGA | GGACTGCTCGAATCAGACGACCCAGTCGCCGTCCTCGCCGGCGA | |
| | GGGTGAGCTCCTCGTCGAATCCCCCTTCCTC | |
| | GGGTCGTCTGATTCGAG | 5615 |
| | CTCGAATCAGACGACCC | 5616 |
| White leaves | GGGGGATTCGACGAGGAGCTCACCCTCGCCGGCGAGGACGGCG | 5617 |
| Immutans | ACTGGGTCGTCAGATTCTAGCAGTCCTTCAACGTATTCCTCACGGA | |
| Triticum aestivum | TACTGTCATCTTTATACTCGATATTCTGTATC | |
| Glu27Term | GATACAGAATATCGAGTATAAAGATGACAGTATCCGTGAGGAATAC | 5618 |
| GAG-TAG | GTTGAAGGACTGCTAGAATCTGACGACCCAGTCGCCGTCCTCGCC | |
| | GGCGAGGGTGAGCTCCTCGTCGAATCCCCC | |
| | TCAGATTCTAGCAGTCC | 5619 |
| | GGACTGCTAGAATCTGA | 5620 |
| White leaves | GGATTCGACGAGGAGCTCACCCTCGCCGGCGAGGACGGCGACTG | 5621 |
| Immutans | GGTCGTCAGATTCGAGTAGTCCTTCAACGTATTCCTCACGGATACT | |
| Triticum aestivum | GTCATCTTTATACTCGATATTCTGTATCGTG | |
| Gln28Term | CACGATACAGAATATCGAGTATAAAGATGACAGTATCCGTGAGGAA | 5622 |
| CAG-TAG | TACGTTGAAGGACTACTCGAATCTGACGACCCAGTCGCCGTCCTC | |
| | GCCGGCGAGGGTGAGCTCCTCGTCGAATCC | |
| | GATTCGAGTAGTCCTTC | 5623 |
| | GAAGGACTACTCGAATC | 5624 |
| White leaves | CGAGCAGTCCTTCAACGTATTCCTCACGGATACTGTCATCTTTATA | 5625 |
| Immutans | CTCGATATTCTGTAGCGTGACCGCGACTACGCAAGGTTCTTCGTG | |
| Triticum aestivum | CTCGAGACCATCGCCAGGGTGCCCTATTTC | |
| Tyr46Term | GAAATAGGGCACCCTGGCGATGGTCTCGAGCACGAAGAACCTTG | 5626 |
| TAT-TAG | CGTAGTCGCGGTCACGCTACAGAATATCGAGTATAAAGATGACAG | |
| | TATCCGTGAGGAATACGTTGAAGGACTGCTCG | |
| | ATTCTGTAGCGTGACCG | 5627 |
| | CGGTCACGCTACAGAAT | 5628 |

### EXAMPLE 30

### Altering Amino Acid Content of Plants

Another aim of biotechnology is to generate plants, especially crop plants, with added value traits. An example of such a trait is improved nutritional quality in food crops. For example, lysine, tryptophan and threonine, which are essential amino acids in the diet of humans and many animals, are limiting nutrients in most cereal crops. Consequently, grain-based diets, such as those based on corn, barley, wheat, rice, maize, millet, sorghum, and the like, must be supplemented with more expensive synthetic amino acids or amino-acid-containing oilseed protein meals. Increasing the lysine content of these grains or of any of the feed component crops would result in significant added value.

Naturally occurring mutants of plants that have different levels of particular essential amino acids have been identified. However, these mutants are generally not the result of increased free amino acid, but are instead the result of shifts in the overall protein profile of the grain. For example, in maize, reduced levels of lysine-deficient endosperm proteins (prolamines) are complemented by elevated levels of more lysine-rich proteins (albumins, globulins and glutelins). While nutritionally superior, these mutants are associated with reduced yields and poor grain quality, limiting their agronomic usefulness.

An alternative approach is to generate plants with mutations that render key amino acid biosynthetic enzymes insensitive to feedback inhibition. Many such mutations are known and mutation results in increased free amino acid. The increased production can optionally be coupled to increased expression of an abundant storage protein comprising the chosen amino acid. Alternatively, a normally abundant protein can be engineered to contain more of the target amino acid.

The attached table discloses exemplary oligonucleotide base sequences which can be used to generate site-specific mutations that remove feedback inhibition in plant amino acid biosynthetic enzymes.

TABLE 37
Genome-Altering Oligos Conferring Amino Acid Overproduction

| Phenotype, Gene, | | |
|---|---|---|
| Plant & Targeted | | SEQ ID |
| Alteration | Altering Oligos | NO: |
| Met Overproduction | TATCCTCCAGGATCTTAAGATTTCCTCCTAATTTCGTCCGTCAGCT | 5629 |
| CGS | GAGCATTAAAGCCCATAGAAACTGTAGCAACATCGGTGTTGCACA | |
| Arabidopsis thaliana | GATCGTGGCGGCTAAGTGGTCCAACAACCC | |
| Arg77His | GGGTTGTTGGACCACTTAGCCGCCACGATCTGTGCAACACCGAT | 5630 |
| CGT-CAT | GTTGCTACAGTTTCTATGGGCTTTAATGCTCAGCTGACGGACGAA | |
| | ATTAGGAGGAAATCTTAAGATCCTGGAGGATA | |
| | TAAAGCCCATAGAAACT | 5631 |
| | AGTTTCTATGGGCTTTA | 5632 |
| Met Overproduction | TCTTAAGATTTCCTCCTAATTTCGTCCGTCAGCTGAGCATTAAAGC | 5633 |
| CGS | CCGTAGAAACTGTAACAACATCGGTGTTGCACAGATCGTGGCGG | |
| Arabidopsis thaliana | CTAAGTGGTCCAACAACCCATCCTCCGCGTT | |
| Ser81Asn | AACGCGGAGGATGGGTTGTTGGACCACTTAGCCGCCACGATCTG | 5634 |
| AGC-AAC | TGCAACACCGATGTTGTTACAGTTTCTACGGGCTTTAATGCTCAGC | |
| | TGACGGACGAAATTAGGAGGAAATCTTAAGA | |
| | AAACTGTAACAACATCG | 5635 |
| | CGATGTTGTTACAGTTT | 5636 |
| Met Overproduction | TTTCCTCCTAATTTCGTCCGTCAGCTGAGCATTAAAGCCCGTAGAA | 5637 |
| CGS | ACTGTAGCAACATCAGTGTTGCACAGATCGTGGCGGCTAAGTGGT | |
| Arabidopsis thaliana | CCAACAACCCATCCTCCGCGTTACCTTCGG | |
| Gly84Ser | CCGAAGGTAACGCGGAGGATGGGTTGTTGGACCACTTAGCCGCC | 5638 |
| GGT-AGT | ACGATCTGTGCAACACTGATGTTGCTACAGTTTCTACGGGCTTTAA | |
| | TGCTCAGCTGACGGACGAAATTAGGAGGAAA | |
| | GCAACATCAGTGTTGCA | 5639 |
| | TGCAACACTGATGTTGC | 5640 |
| Met Overproduction | TTCCTCCTAATTTCGTCCGTCAGCTGAGCATTAAAGCCCGTAGAAA | 5641 |
| CGS | CTGTAGCAACATCGATGTTGCACAGATCGTGGCGGCTAAGTGGTC | |
| Arabidopsis thaliana | CAACAACCCATCCTCCGCGTTACCTTCGGC | |
| Gly84Asp | GCCGAAGGTAACGCGGAGGATGGGTTGTTGGACCACTTAGCCGC | 5642 |
| GGT-GAT | CACGATCTGTGCAACATCGATGTTGCTACAGTTTCTACGGGCTTTA | |
| | ATGCTCAGCTGACGGACGAAATTAGGAGGAA | |
| | CAACATCGATGTTGCAC | 5643 |
| | GTGCAACATCGATGTTG | 5644 |
| Met Overproduction | TATCGTCACTCATCCTCCGCTTCCCTCCCAACTTCGTCCGCCAGC | 5645 |
| CGS | TCAGCACCAAGGCCCACCGCAACTGCAGCAACATCGGCGTCGCG | |
| Fragraria vesca | CAGATCGTCGCGGCTTCGTGGTCCAACAAAGA | |
| Arg73His | TCTTTGTTGGACCACGAAGCCGCGACGATCTGCGCGACGCCGAT | 5646 |
| CGC-CAC | GTTGCTGCAGTTGCGGTGGGCCTTGGTGCTGAGCTGGCGGACGA | |
| | AGTTGGGAGGGAAGCGGAGGATGAGTGACGATA | |
| | CAAGGCCCACCGCAACT | 5647 |
| | AGTTGCGGTGGGCCTTG | 5648 |
| Met Overproduction | TCCTCCGCTTCCCTCCCAACTTCGTCCGCCAGCTCAGCACCAAGG | 5649 |
| CGS | CCCGCCGCAACTGCAACAACATCGGCGTCGCGCAGATCGTCGCG | |
| Fragraria vesca | GCTTCGTGGTCCAACAAAGACTCCGACCTTTC | |
| Ser77Asn | GAAAGGTCGGAGTCTTTGTTGGACCACGAAGCCGCGACGATCTG | 5650 |
| AGC-AAC | CGCGACGCCGATGTTGTTGCAGTTGCGGCGGGCCTTGGTGCTGA | |
| | GCTGGCGGACGAAGTTGGGAGGGAAGCGGAGGA | |
| | CAACTGCAACAACATCG | 5651 |
| | CGATGTTGTTGCAGTTG | 5652 |
| Met Overproduction | TTCCCTCCCAACTTCGTCCGCCAGCTCAGCACCAAGGCCCGCCG | 5653 |
| CGS | CAACTGCAGCAACATCAGCGTCGCGCAGATCGTCGCGGCTTCGT | |
| Fragraria vesca | GGTCCAACAAAGACTCCGACCTTTCGGCGGTGC | |
| Gly80Ser | GCACCGCCGAAAGGTCGGAGTCTTTGTTGGACCACGAAGCCGCG | 5654 |
| GGC-AGC | ACGATCTGCGCGACGCTGATGTTGCTGCAGTTGCGGCGGGCCTT | |
| | GGTGCTGAGCTGGCGGACGAAGTTGGGAGGGAA | |
| | GCAACATCAGCGTCGCG | 5655 |
| | CGCGACGCTGATGTTGC | 5656 |
| Met Overproduction | TCCCTCCCAACTTCGTCCGCCAGCTCAGCACCAAGGCCCGCCGC | 5657 |
| CGS | AACTGCAGCAACATCGACGTCGCGCAGATCGTCGCGGCTTCGTG | |
| Fragraria vesca | TCCAACAAAGACTCCGACCTTTCGGCGGTGCC | |
| Gly80Asp | GGCACCGCCGAAAGGTCGGAGTCTTTGTTGGACCACGAAGCCGC | 5658 |
| GGC-GAC | GACGATCTGCGCGACGTCGATGTTGCTGCAGTTGCGGCGGGCCT | |
| | TGGTGCTGAGCTGGCGGACGAAGTTGGGAGGGA | |
| | CAACATCGACGTCGCGC | 5659 |
| | GCGCGACGTCGATGTTG | 5660 |
| Met Overproduction | TCTCCTCCCTCATCCTCCGCTTCCCTCCCAACTTCCAGCGCCAGC | 5661 |
| CGS | TAAGCACCAAGGCGAGCCGCAACTGCAGCAACATCGGCGTCGCG | |
| Glycine max | CAAATCGTCGCCGCTTCGTGGTCGAACAACAG | |
| Arg68His | CTGTTGTTCGACCACGAAGCGGCGACGATTTGCGCGACGCCGAT | 5662 |
| CGC-CAC | GTTGCTGCAGTTGCGGCTCGCCTTGGTGCTTAGCTGGCGCTGGA | |
| | AGTTGGGAGGGAAGCGGAGGATGAGGGAGGAGA | |
| | CCAAGGCGAGCCGCAAC | 5663 |
| | GTTGCGGCTCGCCTTGG | 5664 |
| Met Overproduction | TCCTCCGCTTCCCTCCCAACTTCCAGCGCCAGCTAAGCACCAAGG | 5665 |
| CGS | CGCGCCGCAACTGCAACAACATCGGCGTCGCGCAAATCGTCGCC | |
| Glycine max | GCTTCGTGGTCGAACAACAGCGACAACTCTCC | |
| Ser72Asn | GGAGAGTTGTCGCTGTTGTTCGACCACGAAGCGGCGACGATTTG | 5666 |
| AGC-AAC | CGCGACGCCGATGTTGTTGCAGTTGCGGCGCGCCTTGGTGCTTA | |
| | GCTGGCGCTGGAAGTTGGGAGGGAAGCGGAGGA | |
| | CAACTGCAACAACATCG | 5667 |
| | CGATGTTGTTGCAGTTG | 5668 |
| Met Overproduction | TTCCCTCCCAACTTCCAGCGCCAGCTAAGCACCAAGGCGCGCCG | 5669 |
| CGS | CAACTGCAGCAACATCAGCGTCGCGCAAATCGTCGCCGCTTCGT | |
| Glycine max | GGTCGAACAACAGCGACAACTCTCCGGCCGCCG | |
| Gly75Ser | CGGCGGCCGGAGAGTTGTCGCTGTTGTTCGACCACGAAGCGGCG | 5670 |
| GGC-AGC | ACGATTTGCGCGACGCTGATGTTGCTGCAGTTGCGGCGCGCCTT | |
| | GGTGCTTAGCTGGCGCTGGAAGTTGGGAGGGAA | |
| | GCAACATCAGCGTCGCG | 5671 |
| | CGCGACGCTGATGTTGC | 5672 |
| Met Overproduction | TCCCTCCCAACTTCCAGCGCCAGCTAAGCACCAAGGCGCGCCGC | 5673 |
| CGS | AACTGCAGCAACATCGACGTCGCGCAAATCGTCGCCGCTTCGTG | |
| Glycine max | GTCGAACAACAGCGACAACTCTCCGGCCGCCGG | |
| Gly75Asp | CCGGCGGCCGGAGAGTTGTCGCTGTTGTTCGACCACGAAGCGGC | 5674 |
| GGC-GAC | GACGATTTGCGCGACGTCGATGTTGCTGCAGTTGCGGCGCGCCT | |
| | TGGTGCTTAGCTGGCGCTGGAAGTTGGGAGGGA | |
| | CAACATCGACGTCGCGC | 5675 |
| | GCGCGACGTCGATGTTG | 5676 |
| Met Overproduction | TGTCTTCTCTGATTTTCAGGTTTCCTCCTAATTTCGTGAGGCAGCT | 5677 |
| CGS | AAGCATTAAGGCTCACAGGAATTGCAGCAATATTGGCGTGGCTCA | |
| Solanum tuberosum | AGTTGTGGCGGCTTCCTGGTCTAACAACCA | |
| Arg70His | TGGTTGTTAGACCAGGAAGCCGCCACAACTTGAGCCACGCCAATA | 5678 |
| AGG-CAC | TTGCTGCAATTCCTGTGAGCCTTAATGCTTAGCTGCCTCACGAAAT | |
| | TAGGAGGAAACCTGAAAATCAGAGAAGACA | |
| | TAAGGCTCACAGGAATT | 5679 |
| | AATTCCTGTGAGCCTTA | 5680 |
| Met Overproduction | TTTTCAGGTTTCCTCCTAATTTCGTGAGGCAGCTAAGCATTAAGGC | 5681 |
| CGS | TAGGAGGAATTGCAACAATATTGGCGTGGCTCAAGTTGTGGCGG | |
| Solanum tuberosum | CTTCCTGGTCTAACAACCAAGCCGGTCCTGA | |
| Ser74Asn | TCAGGACCGGCTTGGTTGTTAGACCAGGAAGCCGCCACAACTTG | 5682 |
| AGC-AAC | AGCCACGCCAATATTGTTGCAATTCCTCCTAGCCTTAATGCTTAGC | |
| | TGCCTCACGAAATTAGGAGGAAACCTGAAAA | |
| | GAATTGCAACAATATTG | 5683 |
| | CAATATTGTTGCAATTC | 5684 |
| Met Overproduction | TTTCCTCCTAATTTCGTGAGGCAGCTAAGCATTAAGGCTAGGAGG | 5685 |
| CGS | AATTGCAGCAATATTAGCGTGGCTCAAGTTGTGGCGGCTTCCTGG | |
| Solanum tuberosum | TCTAACAACCAAGCCGGTCCTGAATTCACTC | |
| Gly77Ser | GAGTGAATTCAGGACCGGCTTGGTTGTTAGACCAGGAAGCCGCC | 5686 |
| GGC-AGC | ACAACTTGAGCCACGCTAATATTGCTGCAATTCCTCCTAGCCTTAA | |
| | TGCTTAGCTGCCTCACGAAATTAGGAGGAAA | |
| | GCAATATTAGCGTGGCT | 5687 |
| | AGCCACGCTAATATTGC | 5688 |
| Met Overproduction | TTCCTCCTAATTTCGTGAGGCAGCTAAGCATTAAGGCTAGGAGGA | 5689 |
| CGS | ATTGCAGCAATATTGACGTGGCTCAAGTTGTGGCGGCTTCCTGGT | |
| Solanum tuberosum | CTAACAACCAAGCCGGTCCTGAATTCACTCC | |
| Gly77Asp | GGAGTGAATTCAGGACCGGCTTGGTTGTTAGACCAGGAAGCCGC | 5690 |
| GGC-GAC | CACAACTTGAGCCACGTCAATATTGCTGCAATTCCTCCTAGCCTTA | |
| | ATGCTTAGCTGCCTCACGAAATTAGGAGGAA | |
| | CAATATTGACGTGGCTC | 5691 |
| | GAGCCACGTCAATATTG | 5692 |
| Met Overproduction | CTTCCTCTCTTATCCTTCGCTTTCCTCCCAACTTTGTCCGTCAGCT | 5693 |
| CGS | CAGCACCAAGGCTCGCCACAACTGCAGCAACATTGGTGTCGCAC | |
| Mesembryanthemum | AGGTCGTCGCTGCCTCCTGGTCCAACAACTC | |
| crystallinum | GAGTTGTTGGACCAGGAGGCAGCGACGACCTGTGCGACACCAAT | 5694 |
| Arg73His | GTTGCTGCAGTTGTGGCGAGCCTTGGTGCTGAGCTGACGGACAA | |
| CGC-CAC | AGTTGGGAGGAAAGCGAAGGATAAGAGAGGAAG | |
| | GGCTCGCCACAACTGCA | 5695 |
| | TGCAGTTGTGGCGAGCC | 5696 |
| Met Overproduction | TCCTTCGCTTTCCTCCCAACTTTGTCCGTCAGCTCAGCACCAAGG | 5697 |
| CGS | CTCGCCGCAACTGCAACAACATTGGTGTCGCACAGGTCGTCGCT | |
| Mesembryanthemum | GCCTCCTGGTCCAACAACTCCGATGCCGGCGC | |
| crystallinum | GCGCCGGCATCGGAGTTGTTGGACCAGGAGGCAGCGACGACCT | 5698 |
| Ser77Asn | GTGCGACACCAATGTTGTTGCAGTTGCGGCGAGCCTTGGTGCTG | |
| AGC-AAC | AGCTGACGGACAAAGTTGGGAGGAAAGCGAAGGA | |
| | CAACTGCAACAACATTG | 5699 |
| | CAATGTTGTTGCAGTTG | 5700 |
| Met Overproduction | TTTCCTCCCAACTTTGTCCGTCAGCTCAGCACCAAGGCTCGCCGC | 5701 |
| CGS | AACTGCAGCAACATTAGTGTCGCACAGGTCGTCGCTGCCTCCTG | |
| Mesembryanthemum | GTCCAACAACTCCGATGCCGGCGCCACCTCTT | |
| crystallinum | AAGAGGTGGCGCCGGCATCGGAGTTGTTGGACCAGGAGGCAGC | 5702 |
| Gly80Ser | GACGACCTGTGCGACACTAATGTTGCTGCAGTTGCGGCGAGCCT | |
| GGT-AGT | TGGTGCTGAGCTGACGGACAAAGTTGGGAGGAAA | |
| | GCAACATTAGTGTCGCA | 5703 |
| | TGCGACACTAATGTTGC | 5704 |
| Met Overproduction | TTCCTCCCAACTTTGTCCGTCAGCTCAGCACCAAGGCTCGCCGCA | 5705 |
| CGS | ACTGCAGCAACATTGATGTCGCACAGGTCGTCGCTGCCTCCTGGT | |
| Mesembryanthemum | CCAACAACTCCGATGCCGGCGCCACCTCTTG | |
| crystallinum | CAAGAGGTGGCGCCGGCATCGGAGTTGTTGGACCAGGAGGCAG | 5706 |
| Gly80Asp | CGACGACCTGTGCGACATCAATGTTGCTGCAGTTGCGGCGAGCC | |
| GGT-GAT | TTGGTGCTGAGCTGACGGACAAAGTTGGGAGGAA | |
| | CAACATTGATGTCGCAC | 5707 |
| | GTGCGACATCAATGTTG | 5708 |
| Met Overproduction | CCTCTGCTACCATCCTCCGCTTTCCGCCAAACTTTGTCCGCCAGC | 5709 |
| CGS | TTAGCACCAAGGCACACCGCAACTGCAGCAACATCGGCGTCGCG | |
| Zea mays | CAGATCGTCGCCGCCGCGTGGTCCGACTGCCC | |
| Arg41His | GGGCAGTCGGACCACGCGGCGGCGACGATCTGCGCGACGCCGA | 5710 |
| CGC-CAC | TGTTGCTGCAGTTGCGGTGTGCCTTGGTGCTAAGCTGGCGGACA | |
| | AAGTTTGGCGGAAAGCGGAGGATGGTAGCAGAGG | |
| | CAAGGCACACCGCAACT | 5711 |
| | AGTTGCGGTGTGCCTTG | 5712 |
| Met Overproduction | TCCTCCGCTTTCCGCCAAACTTTGTCCGCCAGCTTAGCACCAAGG | 5713 |
| CGS | CACGCCGCAACTGCAACAACATCGGCGTCGCGCAGATCGTCGCC | |
| Zea mays | GCCGCGTGGTCCGACTGCCCCGCCGCTCGCCC | |
| Ser45Asn | GGGCGAGCGGCGGGGCAGTCGGACCACGCGGCGGCGACGATCT | 5714 |
| AGC-AAC | GCGCGACGCCGATGTTGTTGCAGTTGCGGCGTGCCTTGGTGCTA | |
| | AGCTGGCGGACAAAGTTTGGCGGAAAGCGGAGGA | |
| | CAACTGCAACAACATCG | 5715 |
| | CGATGTTGTTGCAGTTG | 5716 |
| Met Overproduction | TTTCCGCCAAACTTTGTCCGCCAGCTTAGCACCAAGGCACGCCGC | 5717 |
| CGS | AACTGCAGCAACATCAGCGTCGCGCAGATCGTCGCCGCCGCGTG | |
| Zea mays | GTCCGACTGCCCCGCCGCTCGCCCCCACTTAG | |
| Gly48Ser | CTAAGTGGGGGCGAGCGGCGGGGCAGTCGGACCACGCGGCGG | 5718 |
| GGC-AGC | CGACGATCTGCGCGACGCTGATGTTGCTGCAGTTGCGGCGTGCC | |
| | TTGGTGCTAAGCTGGCGGACAAAGTTTGGCGGAAA | |
| | GCAACATCAGCGTCGCG | 5719 |
| | CGCGACGCTGATGTTGC | 5720 |
| Met Overproduction | TTCCGCCAAACTTTGTCCGCCAGCTTAGCACCAAGGCACGCCGCA | 5721 |
| CGS | ACTGCAGCAACATCGACGTCGCGCAGATCGTCGCCGCCGCGTGG | |
| Zea mays | TCCGACTGCCCCGCCGCTCGCCCCCACTTAGG | |
| Gly48Asp | CCTAAGTGGGGGCGAGCGGCGGGGCAGTCGGACCACGCGGCG | 5722 |
| GGC-GAC | GCGACGATCTGCGCGACGTCGATGTTGCTGCAGTTGCGGCGTGC | |
| | CTTGGTGCTAAGCTGGCGGACAAAGTTTGGCGGAA | |
| | CAACATCGACGTCGCGC | 5723 |
| | GCGCGACGTCGATGTTG | 5724 |
| Met Overproduction | GTATGAATGATCTGTGGGTGAAACACTGTGGGATTAGTCATACAG | 5725 |
| TS | GAAGTTTCAAGGATCGTGGAATGACTGTTTTGGTTAGTCAAGTTAA | |
| Arabidopsis thaliana | TCGTCTGAGAAAGATGAAACGACCTGTGGT | |
| Leu205Arg | ACCACAGGTCGTTTCATCTTTCTCAGACGATTAACTTGACTAACCA | 5726 |
| CTT-CGT | AAACAGTCATTCCACGATCCTTGAAACTTCCTGTATGACTAATCCC | |
| | ACAGTGTTTCACCCACAGATCATTCATAC | |
| | CAAGGATCGTGGAATGA | 5727 |
| | TCATTCCACGATCCTTG | 5728 |
| Met Overproduction | GCATGACTGATTTGTGGGTCAAACACTGTGGGATTAGCCATACTG | 5729 |
| TS | GTAGTTTTAAGGATCGTGGGATGACTGTTTTGGTGAGTCAAGTTAA | |
| Solanum tuberosum | TCGCTTGCGGAAAATGCATAAACCGGTTGT | |
| Leu198Arg | ACAACCGGTTTATGCATTTTCCGCAAGCGATTAACTTGACTCACCA | 5730 |
| CTT-CGT | AAACAGTCATCCCACGATCCTTAAAACTACCAGTATGGCTAATCCC | |
| | ACAGTGTTTGACCCACAAATCAGTCATGC | |
| | TAAGGATCGTGGGATGA | 5731 |
| | TCATCCCACGATCCTTA | 5732 |
| Lys Overproduction | TCATTGGGCACACAGTGAACTGCTTTGGCTCTAGAATCAAAGTGA | 5733 |
| DHPS | TAGGCAACACAGGAAACAACTCAACCAGAGAAGCCGTCCACGCA | |
| Zea mays | ACAGAACAGGGATTTGCTGTTGGCATGCATGC | |
| Ser157Asn | GCATGCATGCCAACAGCAAATCCCTGTTCTGTTGCGTGGACGGCT | 5734 |
| AGC-AAC | TCTCTGGTTGAGTTGTTTCCTGTGTTGCCTATCACTTTGATTCTAG | |
| | AGCCAAAGCAGTTCACTGTGTGCCCAATGA | |
| | CACAGGAAACAACTCAA | 5735 |
| | TTGAGTTGTTTCCTGTG | 5736 |
| Lys Overproduction | GCTCTAGAATCAAAGTGATAGGCAACACAGGAAGCAACTCAACCA | 5737 |
| DHPS | GAGAAGCCGTCCACGAAACAGAACAGGGATTTGCTGTTGGCATG | |
| Zea mays | CATGCGGCTCTCCACATCAATCCTTACTACGG | |
| Ala166Val | CCGTAGTAAGGATTGATGTGGAGAGCCGCATGCATGCCAACAGC | 5738 |
| GCA-GAA | AAATCCCTGTTCTGTTTCGTGGACGGCTTCTCTGGTTGAGTTGCTT | |
| | CCTGTGTTGCCTATCACTTTGATTCTAGAGC | |
| | CGTCCACGAAACAGAAC | 5739 |
| | GTTCTGTTTCGTGGACG | 5740 |
| Lys Overproduction | GGCTCTAGAATCAAAGTGATAGGCAACACAGGAAGCAACTCAACC | 5741 |
| DHPS | AGAGAAGCCGTCCACACAACAGAACAGGGATTTGCTGTTGGCAT | |
| Zea mays | GCATGCGGCTCTCCACATCAATCCTTACTACG | |
| Ala166Thr | CGTAGTAAGGATTGATGTGGAGAGCCGCATGCATGCCAACAGCA | 5742 |
| GCA-ACA | AATCCCTGTTCTGTTGTGTGGACGGCTTCTCTGGTTGAGTTGCTTC | |
| | CTGTGTTGCCTATCACTTTGATTCTAGAGCC | |
| | CCGTCCACACAACAGAA | 5743 |
| | TTCTGTTGTGTGGACGG | 5744 |
| Lys Overproduction | TTATTGGGCATACAGTTAACTGCTTTGGCACTAAAATTAAAGTGGT | 5745 |
| DHPS | CGGCAACACAGGAAATAACTCAACAAGGGAGGCTATTCACGCAAC | |
| Oryza sativa | TGAGCAGGGATTCGCTGTAGGTATGCACGC | |
| Ser124Asn | GCGTGCATACCTACAGCGAATCCCTGCTCAGTTGCGTGAATAGCC | 5746 |
| AGT-AAT | TCCCTTGTTGAGTTATTTCCTGTGTTGCCGACCACTTTAATTTTAGT | |
| | GCCAAAGCAGTTAACTGTATGCCCAATAA | |
| | CACAGGAAATAACTCAA | 5747 |
| | TTGAGTTATTTCCTGTG | 5748 |
| Lys Overproduction | GCACTAAAATTAAAGTGGTCGGCAACACAGGAAGTAACTCAACAA | 5749 |
| DHPS | GGGAGGCTATTCACGTAACTGAGCAGGGATTCGCTGTAGGTATG | |
| Oryza sativa | CACGCGGCTCTCCACATCAATCCTTACTACGG | |
| Ala133Val | CCGTAGTAAGGATTGATGTGGAGAGCCGCGTGCATACCTACAGC | 5750 |
| GCA-GTA | GAATCCCTGCTCAGTTACGTGAATAGCCTCCCTTGTTGAGTTACTT | |
| | CCTGTGTTGCCGACCACTTTAATTTTAGTGC | |
| | TATTCACGTAACTGAGC | 5751 |
| | GCTCAGTTACGTGAATA | 5752 |
| Lys Overproduction | GGCACTAAAATTAAAGTGGTCGGCAACACAGGAAGTAACTCAACA | 5753 |
| DHPS | AGGGAGGCTATTCACACAACTGAGCAGGGATTCGCTGTAGGTAT | |
| Oryza sativa | GCACGCGGCTCTCCACATCAATCCTTACTACG | |
| Ala133Thr | CGTAGTAAGGATTGATGTGGAGAGCCGCGTGCATACCTACAGCG | 5754 |
| GCA-ACA | AATCCCTGCTCAGTTGTGTGAATAGCCTCCCTTGTTGAGTTACTTC | |
| | CTGTGTTGCCGACCACTTTAATTTTAGTGCC | |
| | CTATTCACACAACTGAG | 5755 |
| | CTCAGTTGTGTGAATAG | 5756 |
| Lys Overproduction | TCATCGGGCATACTGTTAACTGCTTTGGAGCCAACATTAAAGTGAT | 5757 |
| DHPS 1 | AGGCAACACGGGAAATAACTCAACCAGAGAAGCTGTTCACGCGA | |
| Triticum aestivum | CAGAGCAGGGATTTGCTGTTGGCATGCATGC | |
| Ser165Asn | GCATGCATGCCAACAGCAAATCCCTGCTCTGTCGCGTGAACAGCT | 5758 |
| AGT-AAT | TCTCTGGTTGAGTTATTTCCCGTGTTGCCTATCACTTTAATGTTGG | |
| | CTCCAAAGCAGTTAACAGTATGCCCGATGA | |
| | CACGGGAAATAACTCAA | 5759 |
| | TTGAGTTATTTCCCGTG | 5760 |
| Lys Overproduction | GAGCCAACATTAAAGTGATAGGCAACACGGGAAGTAACTCAACCA | 5761 |
| DHPS 1 | GAGAAGCTGTTCACGTGACAGAGCAGGGATTTGCTGTTGGCATG | |
| Triticum aestivum | CATGCAGCTCTTCATGTCAATCCTTACTACGG | |
| Ala174Val | CCGTAGTAAGGATTGACATGAAGAGCTGCATGCATGCCAACAGCA | 5762 |
| GCG-GTG | AATCCCTGCTCTGTCACGTGAACAGCTTCTCTGGTTGAGTTACTTC | |
| | CCGTGTTGCCTATCACTTTAATGTTGGCTC | |
| | TGTTCACGTGACAGAGC | 5763 |
| | GCTCTGTCACGTGAACA | 5764 |
| Lys Overproduction | GGAGCCAACATTAAAGTGATAGGCAACACGGGAAGTAACTCAACC | 5765 |
| DHPS 1 | AGAGAAGCTGTTCACACGACAGAGCAGGGATTTGCTGTTGGCAT | |
| Triticum aestivum | GCATGCAGCTCTTCATGTCAATCCTTACTACG | |
| Ala174Thr | CGTAGTAAGGATTGACATGAAGAGCTGCATGCATGCCAACAGCAA | 5766 |
| GCG-ACG | ATCCCTGCTCTGTCGTGTGAACAGCTTCTCTGGTTGAGTTACTTCC | |
| | CGTGTTGCCTATCACTTTAATGTTGGCTCC | |
| | CTGTTCACACGACAGAG | 5767 |
| | CTCTGTCGTGTGAACAG | 5768 |
| Lys Overproduction | TCATCGGGCACACTGTTAACTGCTTTGGAACTAACATTAAAGTGAT | 5769 |
| DHPS 2 | AGGCAACACGGGAAATAACTCAACTAGAGAAGCGATTCACGCTTC | |
| Triticum aestivum | AGAGCAGGGATTTGCTGTTGGCATGCATGC | |
| Ser154Asn | GCATGCATGCCAACAGCAAATCCCTGCTCTGAAGCGTGAATCGCT | 5770 |
| AGT-AAT | TCTCTAGTTGAGTTATTTCCCGTGTTGCCTATCACTTTAATGTTAGT | |
| | TCCAAAGCAGTTAACAGTGTGCCCGATGA | |
| | CACGGGAAATAACTCAA | 5771 |
| | TTGAGTTATTTCCCGTG | 5772 |
| Lys Overproduction | GAACTAACATTAAAGTGATAGGCAACACGGGAAGTAACTCAACTA | 5773 |
| DHPS 2 | GAGAAGCGATTCACGTTTCAGAGCAGGGATTTGCTGTTGGCATGC | |
| Triticum aestivum | ATGCAGCTCTCCATGTCAATCCTTACTATGG | |
| Ala163Val | CCATAGTAAGGATTGACATGGAGAGCTGCATGCATGCCAACAGCA | 5774 |
| GCT-GTT | AATCCCTGCTCTGAAACGTGAATCGCTTCTCTAGTTGAGTTACTTC | |
| | CCGTGTTGCCTATCACTTTAATGTTAGTTC | |
| | GATTCACGTTTCAGAGC | 5775 |
| | GCTCTGAAACGTGAATC | 5776 |
| Lys Overproduction | GGAACTAACATTAAAGTGATAGGCAACACGGGAAGTAACTCAACT | 5777 |
| DHPS 2 | AGAGAAGCGATTCACACTTCAGAGCAGGGATTTGCTGTTGGCATG | |
| Triticum aestivum | CATGCAGCTCTCCATGTCAATCCTTACTATG | |
| Ala163Thr | CATAGTAAGGATTGACATGGAGAGCTGCATGCATGCCAACAGCAA | 5778 |
| GCT-ACT | ATCCCTGCTCTGAAGTGTGAATCGCTTCTCTAGTTGAGTTACTTCC | |
| | CGTGTTGCCTATCACTTTAATGTTAGTTCC | |
| | CGATTCACACTTCAGAG | 5779 |
| | CTCTGAAGTGTGAATCG | 5780 |
| Lys Overproduction | CTCATTGGGCATACTGTGAACTGCTTTGGCTCTAGAATTAAAGTGA | 5781 |
| DHPS | TAGGCAACACAGGAAATAACTCAACCAGAGAAGCTGTTCACGCAA | |
| Coix lacryma-jobi | CAGAGCAGGGATTTGCTGTTGGCATGCATG | |
| Ser154Asn | CATGCATGCCAACAGCAAATCCCTGCTCTGTTGCGTGAACAGCTT | 5782 |
| AGT-AAT | CTCTGGTTGAGTTATTTCCTGTGTTGCCTATCACTTTAATTCTAGA | |
| | GCCAAAGCAGTTCACAGTATGCCCAATGAG | |
| | CACAGGAAATAACTCAA | 5783 |
| | TTGAGTTATTTCCTGTG | 5784 |
| Lys Overproduction | GCTCTAGAATTAAAGTGATAGGCAACACAGGAAGTAACTCAACCA | 5785 |
| DHPS | GAGAAGCTGTTCACGTAACAGAGCAGGGATTTGCTGTTGGCATGC | |
| Coix lacryma-jobi | ATGCAGCTCTCCACATCAATCCTTACTATGG | |
| Ala163Val | CCATAGTAAGGATTGATGTGGAGAGCTGCATGCATGCCAACAGCA | 5786 |
| GCA-GTA | AATCCCTGCTCTGTTACGTGAACAGCTTCTCTGGTTGAGTTACTTC | |
| | CTGTGTTGCCTATCACTTTAATTCTAGAGC | |
| | TGTTCACGTAACAGAGC | 5787 |
| | GCTCTGTTACGTGAACA | 5788 |
| Lys Overproduction | GGCTCTAGAATTAAAGTGATAGGCAACACAGGAAGTAACTCAACC | 5789 |
| DHPS | AGAGAAGCTGTTCACACAACAGAGCAGGGATTTGCTGTTGGCATG | |
| Coix lacryma-jobi | CATGCAGCTCTCCACATCAATCCTTACTATG | |
| Ala163Thr | CATAGTAAGGATTGATGTGGAGAGCTGCATGCATGCCAACAGCAA | 5790 |
| GCA-ACA | ATCCCTGCTCTGTTGTGTGAACAGCTTCTCTGGTTGAGTTACTTCC | |
| | TGTGTTGCCTATCACTTTAATTCTAGAGCC | |
| | CTGTTCACACAACAGAG | 5791 |
| | CTCTGTTGTGTGAACAG | 5792 |
| Lys Overproduction | TCATTGGTCACACAGTCAATTGTTTTGGAGGGTCCATCAAAGTCAT | 5793 |
| DHPS | CGGGAACACTGGAAACAACTCCACAAGGGAAGCAATCCATGCAA | |
| Nicotiana tabacum | CTGAACAGGGATTTGCTGTAGGTATGCATGC | |
| Ser136Asn | GCATGCATACCTACAGCAAATCCCTGTTCAGTTGCATGGATTGCTT | 5794 |
| AGC-AAC | CCCTTGTGGAGTTGTTTCCAGTGTTCCCGATGACTTTGATGGACC | |
| | CTCCAAAACAATTGACTGTGTGACCAATGA | |
| | CACTGGAAACAACTCCA | 5795 |
| | TGGAGTTGTTTCCAGTG | 5796 |
| Lys Overproduction | GAGGGTCCATCAAAGTCATCGGGAACACTGGAAGCAACTCCACAA | 5797 |
| DHPS | GGGAAGCAATCCATGTAACTGAACAGGGATTTGCTGTAGGTATGC | |
| Nicotiana tabacum | ATGCAGCTCTTCACATTAATCCCTACTATGG | |
| Ala145Val | CCATAGTAGGGATTAATGTGAAGAGCTGCATGCATACCTACAGCA | 5798 |
| GCA-GTA | AATCCCTGTTCAGTTACATGGATTGCTTCCCTTGTGGAGTTGCTTC | |
| | CAGTGTTCCCGATGACTTTGATGGACCCTC | |
| | AATCCATGTAACTGAAC | 5799 |
| | GTTCAGTTACATGGATT | 5800 |
| Lys Overproduction | GGAGGGTCCATCAAAGTCATCGGGAACACTGGAAGCAACTCCAC | 5801 |
| DHPS | AAGGGAAGCAATCCATACAACTGAACAGGGATTTGCTGTAGGTAT | |
| Nicotiana tabacum | GCATGCAGCTCTTCACATTAATCCCTACTATG | |
| Ala145Thr | CATAGTAGGGATTAATGTGAAGAGCTGCATGCATACCTACAGCAA | 5802 |
| GCA-ACA | ATCCCTGTTCAGTTGTATGGATTGCTTCCCTTGTGGAGTTGCTTCC | |
| | AGTGTTCCCGATGACTTTGATGGACCCTCC | |
| | CAATCCATACAACTGAA | 5803 |
| | TTCAGTTGTATGGATTG | 5804 |
| Lys Overproduction | TTATAGGCCATACCGTTAACTGTTTTGGCGGAAGCATCAAAGTCAT | 5805 |
| DHPS | TGGAAACACTGGAAACAATTCGACTAGAGAAGCAATCCACGCGAC | |
| Arabidopsis thaliana | TGAACAAGGATTCGCGGTTGGAATGCATGC | |
| Ser142Asn | GCATGCATTCCAACCGCGAATCCTTGTTCAGTCGCGTGGATTGCT | 5806 |
| AGC-AAC | TCTCTAGTCGAATTGTTTCCAGTGTTTCCAATGACTTTGATGCTTC | |
| | CGCCAAAACAGTTAACGGTATGGCCTATAA | |
| | CACTGGAAACAATTCGA | 5807 |
| | TCGAATTGTTTCCAGTG | 5808 |
| Lys Overproduction | GCGGAAGCATCAAAGTCATTGGAAACACTGGAAGCAATTCGACTA | 5809 |
| DHPS | GAGAAGCAATCCACGTGACTGAACAAGGATTCGCGGTTGGAATG | |
| Arabidopsis thaliana | CATGCTGCTCTTCATATAAACCCTTACTATGG | |
| Ala151Val | CCATAGTAAGGGTTTATATGAAGAGCAGCATGCATTCCAACCGCG | 5810 |
| GCG-GTG | AATCCTTGTTCAGTCACGTGGATTGCTTCTCTAGTCGAATTGCTTC | |
| | CAGTGTTTCCAATGACTTTGATGCTTCCGC | |
| | AATCCACGTGACTGAAC | 5811 |
| | GTTCAGTCACGTGGATT | 5812 |
| Lys Overproduction | GGCGGAAGCATCAAAGTCATTGGAAACACTGGAAGCAATTCGACT | 5813 |
| DHPS | AGAGAAGCAATCCACACGACTGAACAAGGATTCGCGGTTGGAAT | |
| Arabidopsis thallana | GCATGCTGCTCTTCATATAAACCCTTACTATG | |
| Ala151Thr | CATAGTAAGGGTTTATATGAAGAGCAGCATGCATTCCAACCGCGA | 5814 |
| GCG-ACG | ATCCTTGTTCAGTCGTGTGGATTGCTTCTCTAGTCGAATTGCTTCC | |
| | AGTGTTTCCAATGACTTTGATGCTTCCGCC | |
| | CAATCCACACGACTGAA | 5815 |
| | TTCAGTCGTGTGGATTG | 5816 |
| Lys Overproduction | TTATTGCTCATACAGTCAACTGTTTTGGTGGGAAAATTAAGGTTAT | 5817 |
| DHPS | TGGAAATACTGGAAACAACTCCACCAGGGAAGCAATTCATGCCAC | |
| Glycine max | TGAGCAGGGTTTTGCTGTTGGAATGCATGC | |
| Ser103Asn | GCATGCATTCCAACAGCAAAACCCTGCTCAGTGGCATGAATTGCT | 5818 |
| AGC-AAC | TCCCTGGTGGAGTTGTTTCCAGTATTTCCAATAACCTTAATTTTCC | |
| | CACCAAAACAGTTGACTGTATGAGCAATAA | |
| | TACTGGAAACAACTCCA | 5819 |
| | TGGAGTTGTTTCCAGTA | 5820 |
| Lys Overproduction | GTGGGAAAATTAAGGTTATTGGAAATACTGGAAGCAACTCCACCA | 5821 |
| DHPS | GGGAAGCAATTCATGTCACTGAGCAGGGTTTTGCTGTTGGAATGC | |
| Glycine max | ATGCTGCCCTTCACATAAACCCTTACTATGG | |
| Ala112Val | CCATAGTAAGGGTTTATGTGAAGGGCAGCATGCATTCCAACAGCA | 5822 |
| GCC-GTC | AAACCCTGCTCAGTGACATGAATTGCTTCCCTGGTGGAGTTGCTT | |
| | CCAGTATTTCCAATAACCTTAATTTTCCCAC | |
| | AATTCATGTCACTGAGC | 5823 |
| | GCTCAGTGACATGAATT | 5824 |
| Lys Overproduction | GGTGGGAAAATTAAGGTTATTGGAAATACTGGAAGCAACTCCACC | 5825 |
| DHPS | AGGGAAGCAATTCATACCACTGAGCAGGGTTTTGCTGTTGGAATG | |
| Glycine max | CATGCTGCCCTTCACATAAACCCTTACTATG | |
| Ala112Thr | CATAGTAAGGGTTTATGTGAAGGGCAGCATGCATTCCAACAGCAA | 5826 |
| GCC-ACC | AACCCTGCTCAGTGGTATGAATTGCTTCCCTGGTGGAGTTGCTTC | |
| | CAGTATTTCCAATAACCTTAATTTTCCCACC | |
| | CAATTCATACCACTGAG | 5827 |
| | CTCAGTGGTATGAATTG | 5828 |
| Trp Overproduction | CTTGCAGGAGACATATTTCAGATCGTGCTGAGTCAACGTTTTGAG | 5829 |
| AS | CGGCGAACATTTGCAAACCCCTTTGAAGTTTATAGAGCACTAAGA | |
| Arabidopsis thallana | GTTGTGAATCCAAGTCCGTATATGGGTTATT | |
| Asp341Asn | AATAACCCATATACGGACTTGGATTCACAACTCTTAGTGCTCTATA | 5830 |
| GAC-AAC | AACTTCAAAGGGGTTTGCAAATGTTCGCCGCTCAAAACGTTGACT | |
| | CAGCACGATCTGAAATATGTCTCCTGCAAG | |
| | CATTTGCAAACCCCTTT | 5831 |
| | AAAGGGGTTTGCAAATG | 5832 |
| Trp Overproduction | GCTGCAGGAGACATATTTCAAATCGTTTTAAGTCAACGCTTTGAGA | 5833 |
| AS | GAAGAACATTTGCTAACCCATTTGAAGTGTACAGAGCATTAAGAAT | |
| Nicotiana tabacum | TGTGAATCCAAGGCCATATATGACTTACA | |
| Asp326Asn | TGTAAGTCATATATGGGCTTGGATTCACAATTCTTAATGCTCTGTA | 5834 |
| GAC-AAC | CACTTCAAATGGGTTAGCAAATGTTCTTCTCTCAAAGCGTTGACTT | |
| | AAAACGATTTGAAATATGTCTCCTGCAGC | |
| | CATTTGCTAACCCATTT | 5835 |
| | AAATGGGTTAGCAAATG | 5836 |
| Trp Overproduction | CTAGCTGGTGACATTTTTCAAGTAGTCTTAAGCCAGCGTTTTGAGA | 5837 |
| AS | GGCGTACATTTGCTAACCCCTTTGAGGTGTACCGTGCATTGCGTA | |
| Orvza sativa | TTGTCAATCCTAGTCCTTATATGGCCTATC | |
| Asp323Asn | GATAGGCCATATAAGGACTAGGATTGACAATACGCAATGCACGGT | 5838 |
| GAC-AAG | ACACCTCAAAGGGGTTAGCAAATGTACGCCTCTCAAAACGCTGGC | |
| | TTAAGACTACTTGAAAAATGTCACCAGCTAG | |
| | CATTTGCTAACCCCTTT | 5839 |
| | AAAGGGGTTAGCAAATG | 5840 |
| Trp Overproduction | CTTGCTGGTGACATATTCCAGATCGTACTAAGTCAGCGTTTTGAAA | 5841 |
| AS | GGCGAACGTTCGCAAACCCATTTGAAATCTATAGATCACTGAGGA | |
| Ruta graveolens | TTGTTAATCCAAGCCCATATATGACTTATT | |
| Asp354Asn | AATAAGTCATATATGGGCTTGGATTAACAATCCTCAGTGATCTATA | 5842 |
| GAC-AAC | GATTTCAAATGGGTTTGCGAACGTTCGCCTTTCAAAACGCTGACTT | |
| | AGTACGATCTGGAATATGTCACCAGCAAG | |
| | CGTTCGCAAACCCATTT | 5843 |
| | AAATGGGTTTGCGAACG | 5844 |
| Trp Overproduction | CTGGCTGGGGACATATTCCAGCTTGTCCTAAGTCAGCGTTTTGAA | 5845 |
| AS | CGGCGAACATTTGCAAATCCATTTGAAGTCTACCGAGCATTGAGA | |
| Catharanthus roseus | ATTGTCAACCCAAGTCCATATATGACTTATT | |
| Asp354Asn | AATAAGTCATATATGGACTTGGGTTGACAATTCTCAATGCTCGGTA | 5846 |
| GAT-AAT | GACTTCAAATGGATTTGCAAATGTTCGCCGTTCAAAACGCTGACTT | |
| | AGGACAAGCTGGAATATGTCCCCAGCCAG | |
| | CATTTGCAAATCCATTT | 5847 |
| | AAATGGATTTGCAAATG | 5848 |

### EXAMPLE 31

### Production of Modified Starch in Plants

A principal aim of biotechnology is the improvement of crop plants for food value, agriculture, and to produce a range of plant-derived raw materials. Along with oils, fats and proteins, polysaccharides constitute the main raw materials derived from plants, and apart from cellulose, the storage polymer starch is the most important polysaccharide raw material. Starch is derived from a range of plants, but maize is the most important cultivated plant for the production of starch.

The polysaccharide starch is a polymer made up of glucose molecules. However, starch is not a homogeneous raw material and is, in fact, a highly complex mixture of various types of molecules which differ from each other, for example, in their degree of polymerization and in the degree of branching of the glucose chains. For example, amylose-starch is a basically non-branched polymer made up of a-1,4-glycosidically branched glucose molecules, and amylopectin-starch is a complex mixture of variously branched glucose chains. The branching results from additional a-1,6-glycosidic linkages. In plants from which starch is typically isolated, for example maize or potato, the starch is approximately 25% amylose-starch and 75% amylopectin-starch.

In maize, various mutants in starch metabolism are known, for example waxy, sugary, shrunken and opaque-2. In addition to producing a modified starch, these mutations greatly improve grain quality in maize, and thus expand the use of maize not only as the food but also for the important industrial materials in food chemistry. It would therefore be advantageous to be able readily to obtain mutants in these genes in particular maize genotypes as well as other plants. Such plants can be obtained, for example, using traditional breeding methods and through specific genetic modification by means of recombinant DNA techniques.

The attached tables disclose exemplary oligonucleotide base sequences which can be used to generate site-specific mutations in genes involved in starch metabolism.

TABLE 38
Genome-Altering Oligos Conferring Increased Starch

| Phenotype, Gene, | | |
|---|---|---|
| Plant & Targeted | | SEQ ID |
| Alteration | Altering Oligos | NO: |
| Increased Starch | GAACTTGAGACTGAGAAAAGGGATCCAAGGACAGTTGCTTCCATT | 5849 |
| ADPGPP | ATTCTTGGAGGTGGAAAAGGAACTCGACTCTTTCCTCTCACAAAA | |
| Arabidopsis thaliana | CGCCGCGCCAAGCCTGCCGTTCCTATCGGGG | |
| Ala99Lys | CCCCGATAGGAACGGCAGGCTTGGCGCGGCGTTTTGTGAGAGGA | 5850 |
| GCA-AAA | AAGAGTCGAGTTCCTTTTCCACCTCCAAGAATAATGGAAGCAACT | |
| | GTCCTTGGATCCCTTTTCTCAGTCTCAAGTTC | |
| | GAGGTGGAAAAGGAACT | 5851 |
| | AGTTCCTTTTCCACCTC | 5852 |
| Increased Starch | CAAAACGCCGCGCCAAGCCTGCCGTTCCTATCGGGGGAGCCTAT | 5853 |
| ADPGPP | AGGTTGATAGATGTACTAATGAGCAATTGTATTAACAGCGGAATCA | |
| Arabidopsis thaliana | ACAAAGTCTACATACTCACACAATATAACTC | |
| Pro127Leu | GAGTTATATTGTGTGAGTATGTAGACTTTGTTGATTCCGCTGTTAA | 5854 |
| CCA-CTA | TACAATTGCTCATTAGTACATCTATCAACCTATAGGCTCCCCCGAT | |
| | AGGAACGGCAGGCTTGGCGCGGCGTTTTG | |
| | AGATGTACTAATGAGCA | 5855 |
| | TGCTCATTAGTACATCT | 5856 |
| Increased Starch | TCACACAATATAACTCAGCATCATTGAACAGGCATTTAGCCCGTGC | 5857 |
| ADPGPP | TTACAACTCCAATAATCTTGGCTTTGGAGATGGCTATGTTGAGGTT | |
| Arabidopsis thaliana | CTTGCGGCCACTCAAACGCCAGGAGAATC | |
| Gly162Asn | GATTCTCCTGGCGTTTGAGTGGCCGCAAGAACCTCAACATAGCCA | 5858 |
| GGA-AAT | TCTCCAAAGCCAAGATTATTGGAGTTGTAAGCACGGGCTAAATGC | |
| | CTGTTCAATGATGCTGAGTTATATTGTGTGA | |
| | CTCCAATAATCTTGGCT | 5859 |
| | AGCCAAGATTATTGGAG | 5860 |
| Increased Starch | TCACACAATATAACTCAGCATCATTGAACAGGCATTTAGCCCGTGC | 5861 |
| ADPGPP | TTACAACTCCAATAACCTTGGCTTTGGAGATGGCTATGTTGAGGTT | |
| Arabidopsis thaliana | CTTGCGGCCACTCAAACGCCAGGAGAATC | |
| Gly162Asn | GATTCTCCTGGCGTTTGAGTGGCCGCAAGAACCTCAACATAGCCA | 5862 |
| GGA-AAC | TCTCCAAAGCCAAGGTTATTGGAGTTGTAAGCACGGGCTAAATGC | |
| | CTGTTCAATGATGCTGAGTTATATTGTGTGA | |
| | CTCCAATAACCTTGGCT | 5863 |
| | AGCCAAGGTTATTGGAG | 5864 |
| Increased Starch | GTTTGAGAGAAGAAAGGTAGACCCGCAAAATGTGGCTGCAATCAT | 5865 |
| ADPGPP | TCTAGGAGGAGGCAAAGGAGCTAAACTCTTCCCTCTTACAATGAG | |
| Arabidopsis thaliana | AGCCGCAACACCAGCTGTAAATATTCATCTT | |
| Asn100Lys | AAGATGAATATTTACAGCTGGTGTTGCGGCTCTCATTGTAAGAGG | 5866 |
| AAT-AAA | GAAGAGTTTAGCTCCTTTGCCTCCTCCTAGAATGATTGCAGCCAC | |
| | ATTTTGCGGGTCTACCTTTCTTCTCTCAAAC | |
| | GGAGGCAAAGGAGCTAA | 5867 |
| | TTAGCTCCTTTGCCTCC | 5868 |
| Increased Starch | CTTGTGTCTTCAAATTATGTTAGGTTCCTGTTGGTGGATGCTACAG | 5869 |
| ADPGPP | GCTGATCGATATCCTGATGAGTAACTGTATTAACAGCTGCATCAAC | |
| Arabidopsis thaliana | AAGATATTTGTGCTGACACAGTTCAACTC | |
| Pro128Leu | GAGTTGAACTGTGTCAGCACAAATATCTTGTTGATGCAGCTGTTAA | 5870 |
| CCG-CTG | TACAGTTACTCATCAGGATATCGATCAGCCTGTAGCATCGACCAA | |
| | CAGGAACCTAACATAATTTGAAGACACAAG | |
| | CGATATCCTGATGAGTA | 5871 |
| | TACTCATCAGGATATCG | 5872 |
| Increased Starch | TGACACAGTTCAACTCAGCTTCCCTTAATCGACATTTAGCACGAAC | 5873 |
| ADPGPP | TTATTTTGGGAATAATATAAACTTTGGAGGTGGTTTCGTAGAGGTA | |
| Arabidopsis thaliana | CAAACACTATGACAATAATAACTCTCAGC | |
| Gly163Asn | GCTGAGAGTTATTATTGTCATAGTGTTTGTACCTCTACGAAACCAC | 5874 |
| GGC-AAT | CTCCAAAGTTTATATTATTCCCAAAATAAGTTCGTGCTAAATGTCG | |
| | ATTAAGGGAAGCTGAGTTGAACTGTGTCA | |
| | TGGGAATAATATAAACT | 5875 |
| | AGTTTATATTATTCCCA | 5876 |
| Increased Starch | TGACACAGTTCAACTCAGCTTCCCTTAATCGACATTTAGCACGAAC | 5877 |
| ADPGPP | TTATTTTGGGAATAACATAAACTTTGGAGGTGGTTTCGTAGAGGTA | |
| Arabidopsis thaliana | CAAACACTATGACAATAATAACTCTCAGC | |
| Gly163Asn | GCTGAGAGTTATTATTGTCATAGTGTTTGTACCTCTACGAAACCAC | 5878 |
| GGC-AAC | CTCCAAAGTTTATGTTATTCCCAAAATAAGTTCGTGCTAAATGTCG | |
| | ATTAAGGGAAGCTGAGTTGAACTGTGTCA | |
| | TGGGAATAACATAAACT | 5879 |
| | AGTTTATGTTATTCCCA | 5880 |
| Increased Starch | TTGAGGAACAACCAACGGCAGATCCAAAAGCTGTTGCCTCTGTCA | 5881 |
| ADPGPP | TTCTAGGTGGTGGTAAAGGAACTCGTCTTTTTCCTCTTACAAGCA | |
| Lycopersicon | GAAGAGCTAAACCAGCTGTTCCTATTGGTGG | |
| esculentum | CCACCAATAGGAACAGCTGGTTTAGCTCTTCTGCTTGTAAGAGGA | 5882 |
| Val94Lys | AAAAGACGAGTTCCTTTACCACCACCTAGAATGACAGAGGCAACA | |
| GTT-AAA | GCTTTTGGATCTGCCGTTGGTTGTTCCTCAA | |
| | TGGTGGTAAAGGAACTC | 5883 |
| | GAGTTCCTTTACCACCA | 5884 |
| Increased Starch | CAAGCAGAAGAGCTAAACCAGCTGTTCCTATTGGTGGTTGTTACC | 5885 |
| ADPGPP | GGCTAATTGATGTACAAATGAGTAACTGCATTAACAGTGGCATAC | |
| Lycopersicon | GGAAAATTTTCATCTTAACACAGTTCAATTC | |
| esculentum | GAATTGAACTGTGTTAAGATGAAAATTTTCCGTATGCCACTGTTAA | 5886 |
| Pro122Leu | TGCAGTTACTCATTTGTACATCAATTAGCCGGTAACAACCACCAAT | |
| CCA-CAA | AGGAACAGCTGGTTTAGCTCTTCTGCTTG | |
| | TGATGTACAAATGAGTA | 5887 |
| | TACTCATTTGTACATCA | 5888 |
| Increased Starch | CACAGTTCAATTCCTTTTCCCTCAATCGTCACCTTGCCCGCACGTA | 5889 |
| ADPGPP | TAATTTTGGAAATAATGTGGGTTTTGGAGATGGATTTGTGGAGGTT | |
| Lycopersicon | TTAGCTGCAACCCAGACTCCAGGGGATGC | |
| esculentum | GCATCCCCTGGAGTCTGGGTTGCAGCTAAAACCTCCACAAATCCA | 5890 |
| Gly158Asn | TCTCCAAAACCCACATTATTTCCAAAATTATACGTGCGGGCAAGGT | |
| GGA-AAT | GACGATTGAGGGAAAAGGAATTGAACTGTG | |
| | TGGAAATAATGTGGGTT | 5891 |
| | AACCCACATTATTTCCA | 5892 |
| Increased Starch | CACAGTTCAATTCCTTTTCCCTCAATCGTCACCTTGCCCGCACGTA | 5893 |
| ADPGPP | TAATTTTGGAAATAACGTGGGTTTTGGAGATGGATTTGTGGAGGT | |
| Lycopersicon | TTTAGCTGCAACCCAGACTCCAGGGGATGC | |
| esculentum | GCATCCCCTGGAGTCTGGGTTGCAGCTAAAACCTCCACAAATCCA | 5894 |
| Gly158Asn | TCTCCAAAACCCACGTTATTTCCAAAATTATACGTGCGGGCAAGGT | |
| GGA-AAC | GACGATTGAGGGAAAAGGAATTGAACTGTG | |
| | TGGAAATAACGTGGGTT | 5895 |
| | AACCCACGTTATTTCCA | 5896 |
| Increased Starch | ACGTAGATTTGGAAAAAAGAGACCCAAGTACAGTTGTAGCAATTAT | 5897 |
| ADPGPP | ACTAGGTGGAGGTAAAGGAACTCGTCTCTTCCCTCTCACCAAGCG | |
| Cicer arietinum | ACGAGCCAAGCCTGCTGTTCCAATTGGAGG | |
| Ala101Lys | CCTCCAATTGGAACAGCAGGCTTGGCTCGTCGCTTGGTGAGAGG | 5898 |
| GCT-AAA | GAAGAGACGAGTTCCTTTACCTCCACCTAGTATAATTGCTACAACT | |
| | GTACTTGGGTCTCTTTTTTCCAAATCTACGT | |
| | TGGAGGTAAAGGAACTC | 5899 |
| | GAGTTCCTTTACCTCCA | 5900 |
| Increased Starch | CCAAGCGACGAGCCAAGCCTGCTGTTCCAATTGGAGGTGCTTATA | 5901 |
| ADPGPP | GGCTGATAGATGTACTAATGAGTAACTGCATCAATAGTGGGATCA | |
| Cicer arietinum | ACAAAGTATACATTCTCACTCAATTTAATTC | |
| Pro129Leu | GAATTAAATTGAGTGAGAATGTATACTTTGTTGATCCCACTATTGA | 5902 |
| CCA-CTA | TGCAGTTACTCATTAGTACATCTATCAGCCTATAAGCACCTCCAAT | |
| | TGGAACAGCAGGCTTGGCTCGTCGCTTGG | |
| | AGATGTACTAATGAGTA | 5903 |
| | TACTCATTAGTACATCT | 5904 |
| Increased Starch | CTCAATTTAATTCAGCCTCACTCAACAGGCATATTGCACGTGCTTA | 5905 |
| ADPGPP | TAACTCTGGTACTAATGTCACTTTTGGAGATGGCTATGTTGAGGTT | |
| Cicer arietinum | CTTGCAGCAACTCAAACTCCAGGGGAGCA | |
| Gly165Asn | TGCTCCCCTGGAGTTTGAGTTGCTGCAAGAACCTCAACATAGCCA | 5906 |
| GGA-AAT | TCTCCAAAAGTGACATTAGTACCAGAGTTATAAGCACGTGCAATAT | |
| | GCCTGTTGAGTGAGGCTGAATTAAATTGAG | |
| | TGGTACTAATGTCACTT | 5907 |
| | AAGTGACATTAGTACCA | 5908 |
| Increased Starch | CTCAATTTAATTCAGCCTCACTCAACAGGCATATTGCACGTGCTTA | 5909 |
| ADPGPP | TAACTCTGGTACTAACGTCACTTTTGGAGATGGCTATGTTGAGGTT | |
| Cicer arietinum | CTTGCAGCAACTCAAACTCCAGGGGAGCA | |
| Gly165Asn | TGCTCCCCTGGAGTTTGAGTTGCTGCAAGAACCTCAACATAGCCA | 5910 |
| GGA-AAC | TCTCCAAAAGTGACGTTAGTACCAGAGTTATAAGCACGTGCAATAT | |
| | GCCTGTTGAGTGAGGCTGAATTAAATTGAG | |
| | TGGTACTAACGTCACTT | 5911 |
| | AAGTGACGTTAGTACCA | 5912 |
| Increased Starch | ATATTGGAGAGGCGTCGGGCAAACCCTAAGAATGTGGCTGCAATC | 5913 |
| ADPGPP | ATACTGCCAGGCGGTAAAGGGACACACCTATTCCCTCTCACCAAT | |
| Ipomoea batatas | CGAGCTGCAACCCCTGCTGTTCCACTTGGAG | |
| Ala94Lys | CTCCAAGTGGAACAGCAGGGGTTGCAGCTCGATTGGTGAGAGGG | 5914 |
| GCA-AAA | AATAGGTGTGTCCCTTTACCGCCTGGCAGTATGATTGCAGCCACA | |
| | TTCTTAGGGTTTGCCCGACGCCTCTCCAATAT | |
| | CAGGCGGTAAAGGGACA | 5915 |
| | TGTCCCTTTACCGCCTG | 5916 |
| Increased Starch | CCAATCGAGCTGCAACCCCTGCTGTTCCACTTGGAGGATGCTATA | 5917 |
| ADPGPP | GGTTGATCGACATTCTAATGAGCAACTGCATCAACAGCGGGGTTA | |
| Ipomoea batatas | ACAAGATCTTTGTGCTGACCCAGTTCAATTC | |
| Pro122Leu | GAATTGAACTGGGTCAGCACAAAGATCTTGTTAACCCCGCTGTTG | 5918 |
| CCA-CTA | ATGCAGTTGCTCATTAGAATGTCGATCAACCTATAGCATCCTCCAA | |
| | GTGGAACAGCAGGGGTTGCAGCTCGATTGG | |
| | CGACATTCTAATGAGCA | 5919 |
| | TGCTCATTAGAATGTCG | 5920 |
| Increased Starch | TGACCCAGTTCAATTCAGCTTCTCTTAACCGTCACATTTCCCGTAC | 5921 |
| ADPGPP | CGTCTTTGGCAATAATGTGAGCTTCGGAGATGGATTTGTTGAGGT | |
| Ipomoea batatas | GCTGGCTGCAACCCAAACACAAGGGGAAAC | |
| Gly157Asn | GTTTCCCCTTGTGTTTGGGTTGCAGCCAGCACCTCAACAAATCCA | 5922 |
| GGT-AAT | TCTCCGAAGCTCACATTATTGCCAAAGACGGTACGGGAAATGTGA | |
| | CGGTTAAGAGAAGCTGAATTGAACTGGGTCA | |
| | TGGCAATAATGTGAGCT | 5923 |
| | AGCTCACATTATTGCCA | 5924 |
| Increased Starch | TGACCCAGTTCAATTCAGCTTCTCTTAACCGTCACATTTCCCGTAC | 5925 |
| ADPGPP | CGTCTTTGGCAATAACGTGAGCTTCGGAGATGGATTTGTTGAGGT | |
| Ipomoea batatas | GCTGGCTGCAACCCAAACACAAGGGGAAAC | |
| Gly157Asn | GTTTCCCCTTGTGTTTGGGTTGCAGCCAGCACCTCAACAAATCCA | 5926 |
| GGT-AAC | TCTCCGAAGCTCACGTTATTGCCAAAGACGGTACGGGAAATGTGA | |
| | CGGTTAAGAGAAGCTGAATTGAACTGGGTCA | |
| | TGGCAATAACGTGAGCT | 5927 |
| | AGCTCACGTTATTGCCA | 5928 |
| Increased Starch | CATTCCGGAGGAACTTTGCGGATCCAAATGAGGTTGCTGCTGTTA | 5929 |
| ADPGPP | TATTGGGTGGTGGCAAAGGGACTCAACTTTTTCCTCTCACAAGCA | |
| Oryza sativa | CAAGGGCCACGCCTGCTGTTCCTATTGGAGG | |
| Thr96Lys | CCTCCAATAGGAACAGCAGGCGTGGCCCTTGTGCTTGTGAGAGG | 5930 |
| ACC-AAA | AAAAAGTTGAGTCCCTTTGCCACCACCCAATATAACAGCAGCAAC | |
| | CTCATTTGGATCCGCAAAGTTCCTCCGGAATG | |
| | TGGTGGCAAAGGGACTC | 5931 |
| | GAGTCCCTTTGCCACCA | 5932 |
| Increased Starch | CAAGCACAAGGGCCACGCCTGCTGTTCCTATTGGAGGATGCTATA | 5933 |
| ADPGPP | GGCTTATCGATATCCTCATGAGCAACTGTTTCAACAGTGGCATAAA | |
| Oryza sativa | CAAGATATTCATAATGACTCAATTCAACTC | |
| Pro124Leu | GAGTTGAATTGAGTCATTATGAATATCTTGTTTATGCCACTGTTGA | 5934 |
| CCC-CTC | AACAGTTGCTCATGAGGATATCGATAAGCCTATAGCATCCTCCAAT | |
| | AGGAACAGCAGGCGTGGCCCTTGTGCTTG | |
| | CGATATCCTCATGAGCA | 5935 |
| | TGCTCATGAGGATATCG | 5936 |
| Increased Starch | TGACTCAATTCAACTCAGCATCTCTTAATCGTCACATTCATCGTAC | 5937 |
| ADPGPP | GTACCTTGGTGGTAATATCAACTTTACTGATGGTTCTGTTGAGGTA | |
| Oryza sativa | TTAGCCGCTACACAAATGCCTGGGGAGGC | |
| Gly159Asn | GCCTCCCCAGGCATTTGTGTAGCGGCTAATACCTCAACAGAACCA | 5938 |
| GGA-AAT | TCAGTAAAGTTGATATTACCACCAAGGTACGTACGATGAATGTGA | |
| | CGATTAAGAGATGCTGAGTTGAATTGAGTCA | |
| | TGGTGGTAATATCAACT | 5939 |
| | AGTTGATATTACCACCA | 5940 |
| Increased Starch | TGACTCAATTCAACTCAGCATCTCTTAATCGTCACATTCATCGTAC | 5941 |
| ADPGPP | GTACCTTGGTGGTAACATCAACTTTACTGATGGTTCTGTTGAGGTA | |
| Oryza sativa | TTAGCCGCTACACAAATGCCTGGGGAGGC | |
| Gly159Asn | GCCTCCCCAGGCATTTGTGTAGCGGCTAATACCTCAACAGAACCA | 5942 |
| GGA-AAC | TCAGTAAAGTTGATGTTACCACCAAGGTACGTACGATGAATGTGA | |
| | CGATTAAGAGATGCTGAGTTGAATTGAGTCA | |
| | TGGTGGTAACATCAACT | 5943 |
| | AGTTGATGTTACCACCA | 5944 |
| Increased Starch | GTCCTTCAGGAGGATTAAGCGATCCGAACGAGGTTGCGGCCGTC | 5945 |
| ADPGPP | ATACTCGGCGGCGGCAAAGGGACTCAGCTCTTCCCACTCACGAG | |
| Triticum aestivum | CACAAGGGCCACACCTGCTGTTCCTATTGGAGG | |
| Thr80Lys | CCTCCAATAGGAACAGCAGGTGTGGCCCTTGTGCTCGTGAGTGG | 5946 |
| ACC-AAA | GAAGAGCTGAGTCCCTTTGCCGCCGCCGAGTATGACGGCCGCAA | |
| | CCTCGTTCGGATCGCTTAATCCTCCTGAAGGAC | |
| | CGGCGGCAAAGGGACTC | 5947 |
| | GAGTCCCTTTGCCGCCG | 5948 |
| Increased Starch | CGAGCACAAGGGCCACACCTGCTGTTCCTATTGGAGGATGTTACA | 5949 |
| ADPGPP | GGCTCATCGACATTCTCATGAGCAACTGCTVCAACAGTGGCATCA | |
| Triticum aestivum | ACAAGATATTCGTCATGACCCAGTTCAACTC | |
| Pro108Leu | GAGTTGAACTGGGTCATGACGAATATCTTGTTGATGCCACTGTTG | 5950 |
| CCC-CTC | AAGCAGTTGCTCATGAGAATGTCGATGAGCCTGTAACATCCTCCA | |
| | ATAGGAACAGCAGGTGTGGCCCTTGTGCTCG | |
| | CGACATTCTCATGAGCA | 5951 |
| | TGCTCATGAGAATGTCG | 5952 |
| Increased Starch | TGACCCAGTTCAACTCGGCCTCCCTTAATCGTCACATTCACCGCA | 5953 |
| ADPGPP | CCTACCTCGGCGGGAATATCAATTTCACTGATGGATCCGTTGAGG | |
| Triticum aestivum | TATTGGCCGCGACGCAAATGCCCGGGGAGGC | |
| Gly143Asn | GCCTCCCCGGGCATTTGCGTCGCGGCCAATACCTCAACGGATCC | 5954 |
| GGA-AAT | ATCAGTGAAATTGATATTCCCGCCGAGGTAGGTGCGGTGAATGTG | |
| | ACGATTAAGGGAGGCCGAGTTGAACTGGGTCA | |
| | CGGCGGGAATATCAATT | 5955 |
| | AATTGATATTCCCGCCG | 5956 |
| Increased Starch | TGACCCAGTTCAACTCGGCCTCCCTTAATCGTCACATTCACCGCA | 5957 |
| ADPGPP | CCTACCTCGGCGGGAACATCAATTTCACTGATGGATCCGTTGAGG | |
| Triticum aestivum | TATTGGCCGCGACGCAAATGCCCGGGGAGGC | |
| Gly143Asn | GCCTCCCCGGGCATTTGCGTCGCGGCCAATACCTCAACGGATCC | 5958 |
| GGA-AAC | ATCAGTGAAATTGATGTTCCCGCCGAGGTAGGTGCGGTGAATGTG | |
| | ACGATTAAGGGAGGCCGAGTTGAACTGGGTCA | |
| | CGGCGGGAACATCAATT | 5959 |
| | AATTGATGTTCCCGCCG | 5960 |
| Increased Starch | CCTCCCGAAAGAATTATGCTGATGCAAGCCACGTTTCTGCTGTCA | 5961 |
| ADPGPP | TTTTGGGTGGAGGCAAAGGAGTTCAACTCTTTCCTCTGACAAGCA | |
| Oryza sativa | CAAGGGCTACCCCCGCTGTTCCTGTTGGAGG | |
| Thr95Lys | CCTCCAACAGGAACAGCGGGGGTAGCCCTTGTGCTTGTCAGAGG | 5962 |
| ACT-AAA | AAAGAGTTGAACTCCTTTGCCTCCACCCAAAATGACAGCAGAAAC | |
| | GTGGCTTGCATCAGCATAATTCTTTCGGGAGG | |
| | TGGAGGCAAAGGAGTTC | 5963 |
| | GAACTCCTTTGCCTCCA | 5964 |
| Increased Starch | CAAGCACAAGGGCTACCCCCGCTGTTCCTGTTGGAGGATGTTACA | 5965 |
| ADPGPP | GGCTTATTGACATCCTTATGAGCAATTGCTTCAATAGCGGAATAAA | |
| Oryza sativa | TAAAATATTTGTGATGACTCAGTTCAATTC | |
| Pro123Leu | GAATTGAACTGAGTCATCACAAATATTTTATTTATTCCGCTATTGAA | 5966 |
| CCT-CTT | GCAATTGCTCATAAGGATGTCAATAAGCCTGTAACATCCTCCAACA | |
| | GGAACAGCGGGGGTAGCCCTTGTGCTTG | |
| | TGACATCCTTATGAGCA | 5967 |
| | TGCTCATAAGGATGTCA | 5968 |
| Increased Starch | TGACTCAGTTCAATTCTGCTTCTCTTAATCGCCATATCCATCATACA | 5969 |
| ADPGPP | TACCTTGGTGGGAATATCAACTTTACTGATGGGTCTGTGCAGGTA | |
| Oryza sativa | TTGGCTGCTACACAAATGCCTGACGAACC | |
| Gly158Asn | GGTTCGTCAGGCATTTGTGTAGCAGCCAATACCTGCACAGACCCA | 5970 |
| GGG-AAT | TCAGTAAAGTTGATATTCCCACCAAGGTATGTATGATGGATATGGC | |
| | GATTAAGAGAAGCAGAATTGAACTGAGTCA | |
| | TGGTGGGAATATCAACT | 5971 |
| | AGTTGATATTCCCACCA | 5972 |
| Increased Starch | TGACTCAGTTCAATTCTGCTTCTCTTAATCGCCATATCCATCATACA | 5973 |
| ADPGPP | TACCTTGGTGGGAACATCAACTTTACTGATGGGTCTGTGCAGGTA | |
| Oryza sativa | TTGGCTGCTACACAAATGCCTGACGAACC | |
| Gly158Asn | GGTTCGTCAGGCATTTGTGTAGCAGCCAATACCTGCACAGACCCA | 5974 |
| GGG-AAC | TCAGTAAAGTTGATGTTCCCACCAAGGTATGTATGATGGATATGG | |
| | CGATTAAGAGAAGCAGAATTGAACTGAGTCA | |
| | TGGTGGGAACATCAACT | 5975 |
| | AGTTGATGTTCCCACCA | 5976 |
| Increased Starch | CCTTCCGCAGGAATTACGCCGATCCGAACGAGGTCGCGGCCGTC | 5977 |
| ADPGPP | ATACTCGGCGGTGGCAAAGGGACTCAGCTCTTCCCTCTCACAAG | |
| Triticum aestivum | CACAAGGGCCACACCTGCTGTTCCTATTGGAGG | |
| Thr99Lys | CCTCCAATAGGAACAGCAGGTGTGGCCCTTGTGCTTGTGAGAGG | 5978 |
| ACC-AAA | GAAGAGCTGAGTCCCTTTGCCACCGCCGAGTATGACGGCCGCGA | |
| | CCTCGTTCGGATCGGCGTAATTCCTGCGGAAGG | |
| | CGGTGGCAAAGGGACTC | 5979 |
| | GAGTCCCTTTGCCACCG | 5980 |
| Increased Starch | CAAGCACAAGGGCCACACCTGCTGTTCCTATTGGAGGATGTTACA | 5981 |
| ADPGPP | GGCTCATCGATATTCTCATGAGCAACTGCTTCAATAGTGGCATCAA | |
| Triticum aestivum | CAAGATATTCGTCATGACGCAGTTCAACTC | |
| Pro127Leu | GAGTTGAACTGCGTCATGACGAATATCTTGTTGATGCCACTATTGA | 5982 |
| CCC-CTC | AGCAGTTGCTCATGAGAATATCGATGAGCCTGTAACATCCTCCAA | |
| | TAGGAACAGCAGGTGTGGCCCTTGTGCTTG | |
| | CGATATTCTCATGAGCA | 5983 |
| | TGCTCATGAGAATATCG | 5984 |
| Increased Starch | TGACGCAGTTCAACTCGGCCTCTCTTAATCGTCACATTCACCGCA | 5985 |
| ADPGPP | CCTACCTCGGCGGGAATATCAATTTCACTGATGGATCTGTTGAGG | |
| Triticum aestivum | TATTGGCCGCGACGCAAATGCCCGGGGAGGC | |
| Gly162Asn | GCCTCCCCGGGCATTTGCGTCGCGGCCAATACCTCAACAGATCC | 5986 |
| GGA-AAT | ATCAGTGAAATTGATATTCCCGCCGAGGTAGGTGCGGTGAATGTG | |
| | ACGATTAAGAGAGGCCGAGTTGAACTGCGTCA | |
| | CGGCGGGAATATCAATT | 5987 |
| | AATTGATATTCCCGCCG | 5988 |
| Increased Starch | TGACGCAGTTCAACTCGGCCTCTCTTAATCGTCACATTCACCGCA | 5989 |
| ADPGPP | CCTACCTCGGCGGGAACATCAATTTCACTGATGGATCTGTTGAGG | |
| Triticum aestivum | TATTGGCCGCGACGCAAATGCCCGGGGAGGC | |
| Gly162Asn | GCCTCCCCGGGCATTTGCGTCGCGGCCAATACCTCAACAGATCC | 5990 |
| GGA-AAC | ATCAGTGAAATTGATGTTCCCGCCGAGGTAGGTGCGGTGAATGTG | |
| | ACGATTAAGAGAGGCCGAGTTGAACTGCGTCA | |
| | CGGCGGGAACATCAATT | 5991 |
| | AATTGATGTTCCCGCCG | 5992 |
| Increased Starch | CTTTTCGGAGGAATTATGCTGATCCTAATGAAGTCGCTGCCGTCA | 5993 |
| ADPGPP | TTTTGGGTGGTGGTAAAGGGACTCAGCTTTTCCCTCTCACAAGCA | |
| Zea mays | CAAGGGCCACCCCTGCTGTTCCTATTGGAGG | |
| Thr96Lys | CCTCCAATAGGAACAGCAGGGGTGGCCCTTGTGCTTGTGAGAGG | 5994 |
| ACC-AAA | GAAAAGCTGAGTCCCTTTACCACCACCCAAAATGACGGCAGCGAC | |
| | TTCATTAGGATCAGCATAATTCCTCCGAAAAG | |
| | TGGTGGTAAAGGGACTC | 5995 |
| | GAGTCCCTTTACCACCA | 5996 |
| Increased Starch | CAAGCACAAGGGCCACCCCTGCTGTTCCTATTGGAGGATGTTACA | 5997 |
| ADPGPP | GGCTTATTGATATCCTCATGAGCAACTGTTTCAACAGTGGCATAAA | |
| Zea mays | CAAGATATTTGTTATGACTCAGTTCAACTC | |
| Pro124Leu | GAGTTGAACTGAGTCATAACAAATATCTTGTTTATGCCACTGTTGA | 5998 |
| CCC-CTC | AACAGTTGCTCATGAGGATATCAATAAGCCTGTAACATCCTCCAAT | |
| | AGGAACAGCAGGGGTGGCCCTTGTGCTTG | |
| | TGATATCCTCATGAGCA | 5999 |
| | TGCTCATGAGGATATCA | 6000 |
| Increased Starch | TGACTCAGTTCAACTCAGCTTCTCTTAACCGTCACATTCATCGTAC | 6001 |
| ADPGPP | CTATCTTGGTGGGAATATCAACTTCACTGATGGATCTGTTGAGGT | |
| Zea mays | GCTGGCTGCAACACAAATGCCTGGGGAGGC | |
| Gly159Asn | GCCTCCCCAGGCATTTGTGTTGCAGCCAGCACCTCAACAGATCCA | 6002 |
| GGG-AAT | TCAGTGAAGTTGATATTCCCACCAAGATAGGTACGATGAATGTGA | |
| | CGGTTAAGAGAAGCTGAGTTGAACTGAGTCA | |
| | TGGTGGGAATATCAACT | 6003 |
| | AGTTGATATTCCCACCA | 6004 |
| Increased Starch | TGACTCAGTTCAACTCAGCTTCTCTTAACCGTCACATTCATCGTAC | 6005 |
| ADPGPP | CTATCTTGGTGGGAACATCAACTTCACTGATGGATCTGTTGAGGT | |
| Zea mays | GCTGGCTGCAACACAAATGCCTGGGGAGGC | |
| Gly159Asn | GCCTCCCCAGGCATTTGTGTTGCAGCCAGCACCTCAACAGATCCA | 6006 |
| GGG-AAC | TCAGTGAAGTTGATGTTCCCACCAAGATAGGTACGATGAATGTGA | |
| | CGGTTAAGAGAAGCTGAGTTGAACTGAGTCA | |
| | TGGTGGGAACATCAACT | 6007 |
| | AGTTGATGTTCCCACCA | 6008 |
| Increased Starch | CTTGAGAGGCAAAAGAAGGGCGATGCAAGGACAGTAGTAGCAAT | 6009 |
| ADPGPP | CATTCTAGGAGGGGGAAAGGGAACTCGTCTTTTCCCCCTCACCAA | |
| Solanum tuberosum | ACGTCGTGCTAAGCCTGCCGTTCCAATGGGAG | |
| Ala58Lys | CTCCCATTGGAACGGCAGGCTTAGCACGACGTTTGGTGAGGGGG | 6010 |
| GCG-AAG | AAAAGACGAGTTCCCTTTCCCCCTCCTAGAATGATTGCTACTACTG | |
| | TCCTTGCATCGCCCTTCTTTTGCCTCTCAAG | |
| | GAGGGGGAAAGGGAACT | 6011 |
| | AGTTCCCTTTCCCCCTC | 6012 |
| Increased Starch | CCAAACGTCGTGCTAAGCCTGCCGTTCCAATGGGAGGAGCATATA | 6013 |
| ADPGPP | GGCTAATTGATGTACTAATGAGCAACTGTATTAACAGTGGCATCAA | |
| Solanum tuberosum | CAAAGTATACATTCTCACTCAATTCAACTC | |
| Pro86Leu | GAGTTGAATTGAGTGAGAATGTATACTTTGTTGATGCCACTGTTAA | 6014 |
| CCA-CTA | TACAGTTGCTCATTAGTACATCAATTAGCCTATATGCTCCTCCCAT | |
| | TGGAACGGCAGGCTTAGCACGACGTTTGG | |
| | TGATGTACTAATGAGCA | 6015 |
| | TGCTCATTAGTACATCA | 6016 |
| Increased Starch | CTCAATTCAACTCAGCCTCACTTAACAGGCATATAGCTCGTGCTTA | 6017 |
| ADPGPP | CAACTTTGGCAATAATGTCACATTCGAGAGTGGCTATGTCGAGGT | |
| Solanum tuberosum | CTTAGCAGCAACTCAAACACCAGGTGAATT | |
| Gly122Asn | AATTCACCTGGTGTTTGAGTTGCTGCTAAGACCTCGACATAGCCA | 6018 |
| GGG-AAT | CTCTCGAATGTGACATTATTGCCAAAGTTGTAAGCACGAGCTATAT | |
| | GCCTGTTAAGTGAGGCTGAGTTGAATTGAG | |
| | TGGCAATAATGTCACAT | 6019 |
| | ATGTGACATTATTGCCA | 6020 |
| Increased Starch | CTCAATTCAACTCAGCCTCACTTAACAGGCATATAGCTCGTGCTTA | 6021 |
| ADPGPP | CAACTTTGGCAATAACGTCACATTCGAGAGTGGCTATGTCGAGGT | |
| Solanum tuberosum | CTTAGCAGCAACTCAAACACCAGGTGAATT | |
| Gly122Asn | AATTCACCTGGTGTTTGAGTTGCTGCTAAGACCTCGACATAGCCA | 6022 |
| GGG-AAC | CTCTCGAATGTGACGTTATTGCCAAAGTTGTAAGCACGAGCTATAT | |
| | GCCTGTTAAGTGAGGCTGAGTTGAATTGAG | |
| | TGGCAATAACGTCACAT | 6023 |
| | ATGTGACGTTATTGCCA | 6024 |
| Increased Starch | TATTTGAATCTCCAAAAGCTGACCCAAAAAATGTGGCTGCAATTGT | 6025 |
| ADPGPP | GCTGGGTGGTGGTAAAGGGACTCGCCTCTTTCCTCTTACTAGCAG | |
| Beta vulgaris | GAGAGCTAAGCCAGCAGTGCCAATTGGAGG | |
| Ala98Lys | CCTCCAATTGGCACTGCTGGCTTAGCTCTCCTGCTAGTAAGAGGA | 6026 |
| GCT-AAA | AAGAGGCGAGTCCCTTTACCACCACCCAGCACAATTGCAGCCACA | |
| | TTTTTTGGGTCAGCTTTTGGAGATTCAAATA | |
| | TGGTGGTAAAGGGACTC | 6027 |
| | GAGTCCCTTTACCACCA | 6028 |
| Increased Starch | TATTTGAATCTCCAAAAGCTGACCCAAAAAATGTGGCTGCAATTGT | 6029 |
| ADPGPP | GCTGGGTGGTGGTAACGGGACTCGCCTCTTTCCTCTTACTAGCAG | |
| Beta vulgaris | GAGAGCTAAGCCAGCAGTGCCAATTGGAGG | |
| Ala98Lys | CCTCCAATTGGCACTGCTGGCTTAGCTCTCCTGCTAGTAAGAGGA | 6030 |
| GCT-AAC | AAGAGGCGAGTCCCGTTACCACCACCCAGCACAATTGCAGCCAC | |
| | ATTTTTTGGGTCAGCTTTTGGAGATTCAAATA | |
| | TGGTGGTAACGGGACTC | 6031 |
| | GAGTCCCGTTACCACCA | 6032 |
| Increased Starch | CTAGCAGGAGAGCTAAGCCAGCAGTGCCAATTGGAGGGTGTTAC | 6033 |
| ADPGPP | AGGCTGATTGATGTGCTTATGAGCAACTGCATCAACAGTGGCATT | |
| Beta vulgaris | AGAAAGATTTTCATTCTTACCCAGTTCAATTC | |
| Pro126Leu | GAATTGAACTGGGTAAGAATGAAAATCTTTCTAATGCCACTGTTGA | 6034 |
| CCT-CTT | TGCAGTTGCTCATAAGCACATCAATCAGCCTGTAACACCCTCCAA | |
| | TTGGCACTGCTGGCTTAGCTCTCCTGCTAG | |
| | TGATGTGCTTATGAGCA | 6035 |
| | TGCTCATAAGCACATCA | 6036 |
| Increased Starch | CCCAGTTCAATTCGTTTTCGCTTAATCGTCATCTTGCTCGAACCTA | 6037 |
| ADPGPP | TAATTTTGGAGATAATGTGAATTTTGGGGATGGCTTTGTGGAGGTT | |
| Beta vulgaris | TTTGCTGCTACACAAACACCTGGAGAATC | |
| Gly162Asn | GATTCTCCAGGTGTTTGTGTAGCAGCAAAAACCTCCACAAAGCCA | 6038 |
| GGT-AAT | TCCCCAAAATTCACATTATCTCCAAAATTATAGGTTCGAGCAAGAT | |
| | GACGATTAAGCGAAAACGAATTGAACTGGG | |
| | TGGAGATAATGTGAATT | 6039 |
| | AATTCACATTATCTCCA | 6040 |
| Increased Starch | CCCAGTTCAATTCGTTTTCGCTTAATCGTCATCTTGCTCGAACCTA | 6041 |
| ADPGPP | TAATTTTGGAGATAACGTGAATTTTGGGGATGGCTTTGTGGAGGT | |
| Beta vulgaris | TTTTGCTGCTACACAAACACCTGGAGAATC | |
| Gly162Asn | GATTCTCCAGGTGTTTGTGTAGCAGCAAAAACCTCCACAAAGCCA | 6042 |
| GGT-AAC | TCCCCAAAATTCACGTTATCTCCAAAATTATAGGTTCGAGCAAGAT | |
| | GACGATTAAGCGAAAACGAATTGAACTGGG | |
| | TGGAGATAACGTGAATT | 6043 |
| | AATTCACGTTATCTCCA | 6044 |

TABLE 39

Oligonucleotides to produce plants with waxy starch
Genome-Altering Oligos Conferring Tncreased Starch

| Phenotype, Gene, | | |
|---|---|---|
| Plant & Targeted | | SEQ ID |
| Alteration | Altering Oligos | NO: |
| Waxy starch | GAATCCAGGTAAACGGGTAGTTCATAATGGCAACTGTGACTGCTT | 6045 |
| GBSS | CTTCTAACTTTGTGTGAAGAACTTCACTTTTCAACAATCATGGTGCT | |
| Arabidopsis thaliana | CTTCATGCTCTGATGTCGCTCAGATTAC | |
| Ser12Term | GTAATCTGAGCGACATCAGAGCATGAAGAAGCACCATGATTGTTG | 6046 |
| TCA-TGA | AAAAGTGAAGTTCTTCACACAAAGTTAGAAGAAGCAGTCACAGTTG | |
| | CCATTATGAACTACCCGTTTACCTGGATTC | |
| | CTTTGTGTGAAGAACTT | 6047 |
| | AAGTTCTTCACACAAAG | 6048 |
| Waxy starch | ATCCAGGTAAACGGGTAGTTCATAATGGCAACTGTGACTGCTTCTT | 6049 |
| GBSS | CTAACTTTGTGTCATGAACTTCACTTTTCAACAATCATGGTGCTTCT | |
| Arabidopsis thaliana | TCATGCTCTGATGTCGCTCAGATTACCT | |
| Arg13Term | AGGTAATCTGAGCGACATCAGAGCATGAAGAAGCACCATGATTGT | 6050 |
| AGA-TGA | TGAAAAGTGAAGTTCATGACACAAAGTTAGAAGAAGCAGTCACAGT | |
| | TGCCATTATGAACTACCCGTTTACCTGGAT | |
| | TTGTGTCATGAACTTCA | 6051 |
| | TGAAGTTCATGACACAA | 6052 |
| Waxy starch | TAAACGGGTAGTTCATAATGGCAACTGTGACTGCTTCTTCTAACTT | 6053 |
| GBSS | TGTGTCAAGAACTTGACTTTTCAACAATCATGGTGCTTCTTCATGCT | |
| Arabidopsis thaliana | CTGATGTCGCTCAGATTACCTTAAAAGG | |
| Ser15Term | CCTTTTAAGGTAATCTGAGCGACATCAGAGCATGAAGAAGCACCAT | 6054 |
| TCA-TGA | GATTGTTGAAAAGTCAAGTTCTTGACACAAAGTTAGAAGAAGCAGT | |
| | CACAGTTGCCATTATGAACTACCCGTTTA | |
| | AAGAACTTGACTTTTCA | 6055 |
| | TGAAAAGTCAAGTTCTT | 6056 |
| Waxy starch | TGACTGCTTCTTCTAACTTTGTGTCAAGAACTTCACTTTTCAACAAT | 6057 |
| GBSS | CATGGTGCTTCTTGATGCTCTGATGTCGCTCAGATTACCTTAAAAG | |
| Arabidopsis thaliana | GCCAATCCTTGACTCATTGTGGGTTAAG | |
| Ser24Term | CTTAACCCACAATGAGTCAAGGATTGGCCTTTTAAGGTAATCTGAG | 6058 |
| TCA-TGA | CGACATCAGAGCATCAAGAAGCACCATGATTGTTGAAAAGTGAAG | |
| | TTCTTGACACAAAGTTAGAAGAAGCAGTCA | |
| | TGCTTCTTGATGCTCTG | 6059 |
| | CAGAGCATCAAGAAGCA | 6060 |
| Waxy starch | TGCTTCTTCTAACTTTGTGTCAAGAACTTCACTTTTCAACAATCATG | 6061 |
| GBSS | GTGCTTCTTCATGATCTGATGTCGCTCAGATTACCTTAAAAGGCCA | |
| Arabidopsis thaliana | ATCCTTGACTCATTGTGGGTTAAGGTCA | |
| Cys25Term | TGACCTTAACCCACAATGAGTCAAGGATTGGCCTTTTAAGGTAATC | 6062 |
| TGC-TGA | TGAGCGACATCAGATCATGAAGAAGCACCATGATTGTTGAAAAGT | |
| | GAAGTTCTTGACACAAAGTTAGAAGAAGCA | |
| | TCTTCATGATCTGATGT | 6063 |
| | ACATCAGATCATGAAGA | 6064 |
| Waxy starch | GTAACAGCTTCACAGTTGGTGTCACATGTCCATGGTGGAGCAACG | 6065 |
| GBSS | TCTTCACCGGATACTTAAACAAACTTGGCCCAGGTTGGCCTCAGG | |
| Antirrhinum majus | AACCAGCAATTCACTCACAATGGGTTGAGAT | |
| Lys24Term | ATCTCAACCCATTGTGAGTGAATTGCTGGTTCCTGAGGCCAACCTG | 6066 |
| AAA-TAA | GGCCAAGTTTGTTTAAGTATCCGGTGAAGACGTTGCTCCACCATG | |
| | GACATGTGACACCAACTGTGAAGCTGTTAC | |
| | CGGATACTTAAACAAAC | 6067 |
| | GTTTGTTTAAGTATCCG | 6068 |
| Waxy starch | CACAGTTGGTGTCACATGTCCATGGTGGAGCAACGTCTTCACCGG | 6069 |
| GBSS | ATACTAAAACAAACTAGGCCCAGGTTGGCCTCAGGAACCAGCAAT | |
| Antirrhinum majus | TCACTCACAATGGGTTGAGATCAATAAACAT | |
| Leu21Term | ATGTTTATTGATCTCAACCCATTGTGAGTGAATTGCTGGTTCCTGA | 6070 |
| TTG-TAG | GGCCAACCTGGGCCTAGTTTGTTTTAGTATCCGGTGAAGACGTTG | |
| | CTCCACCATGGACATGTGACACCAACTGTG | |
| | AACAAACTAGGCCCAGG | 6071 |
| | CCTGGGCCTAGTTTGTT | 6072 |
| Waxy starch | TTGGTGTCACATGTCCATGGTGGAGCAACGTCTTCACCGGATACT | 6073 |
| GBSS | AAAACAAACTTGGCCTAGGTTGGCCTCAGGAACCAGCAATTCACT | |
| Antirrhinum majus | CACAATGGGTTGAGATCAATAAACATGGTTG | |
| Gln29Term | CAACCATGTTTATTGATCTCAACCCATTGTGAGTGAATTGCTGGTT | 6074 |
| CAG-TAG | CCTGAGGCCAACCTAGGCCAAGTTTGTTTTAGTATCCGGTGAAGA | |
| | CGTTGCTCCACCATGGACATGTGACACCAA | |
| | ACTTGGCCTAGGTTGGC | 6075 |
| | GCCAACCTAGGCCAAGT | 6076 |
| Waxy starch | GGTGGAGCAACGTCTTCACCGGATACTAAAACAAACTTGGCCCAG | 6077 |
| GBSS | GTTGGCCTCAGGAACTAGCAATTCACTCACAATGGGTTGAGATCA | |
| Antirrhinum majus | ATAAACATGGTTGATAAGCTTCAAATGAGGA | |
| Gln35Term | TCCTCATTTGAAGCTTATCAACCATGTTTATTGATCTCAACCCATTG | 6078 |
| CAG-TAG | TGAGTGAATTGCTAGTTCCTGAGGCCAACCTGGGCCAAGTTTGTTT | |
| | TAGTATCCGGTGAAGACGTTGCTCCACC | |
| | TCAGGAACTAGCAATTC | 6079 |
| | GAATTGCTAGTTCCTGA | 6080 |
| Waxy starch | GGAGCAACGTCTTCACCGGATACTAAAACAAACTTGGCCCAGGTT | 6081 |
| GBSS | GGCCTCAGGAACCAGTAATTCACTCACAATGGGTTGAGATCAATAA | |
| Antirrhinum majus | ACATGGTTGATAAGCTTCAAATGAGGAACA | |
| Gln36Term | TGTTCCTCATTTGAAGCTTATCAACCATGTTTATTGATCTCAACCCA | 6082 |
| CAA-TAA | TTGTGAGTGAATTACTGGTTCCTGAGGCCAACCTGGGCCAAGTTT | |
| | GTTTTAGTATCCGGTGAAGACGTTGCTCC | |
| | GGAACCAGTAATTCACT | 6083 |
| | AGTGAATTACTGGTTCC | 6084 |
| Waxy starch | GTGATGGCGACTATAACTGCCTCACACTTTGTTTCTCATGTCTGTG | 6085 |
| GBSS | GGGGTGCCACTTCTTGAGAATCAAAAGTGGGGTTGGGTCAATTAG | |
| Ipomoea batatas | CCCTGAGGAGCCAAGCTGTGACTCACAATG | |
| Gly20Term | CATTGTGAGTCACAGCTTGGCTCCTCAGGGCTAATTGACCCAACC | 6086 |
| GGA-TGA | CCACTTTTGATTCTCAAGAAGTGGCACCCCCACAGACATGAGAAA | |
| | CAAAGTGTGAGGCAGTTATAGTCGCCATCAC | |
| | CCACTTCTTGAGAATCA | 6087 |
| | TGATTCTCAAGAAGTGG | 6088 |
| Waxy starch | ATGGCGACTATAACTGCCTCACACTTTGTTTCTCATGTCTGTGGGG | 6089 |
| GBSS | GTGCCACTTCTGGATAATCAAAAGTGGGGTTGGGTCAATTAGCCC | |
| Ipomoea batatas | TGAGGAGCCAAGCTGTGACTCACAATGGGT | |
| Glu21Term | ACCCATTGTGAGTCACAGCTTGGCTCCTCAGGGCTAATTGACCCA | 6090 |
| GAA-TAA | ACCCCACTTTTGATTATCCAGAAGTGGCACCCCCACAGACATGAG | |
| | AAACAAAGTGTGAGGCAGTTATAGTCGCCAT | |
| | CTTCTGGATAATCAAAA | 6091 |
| | TTTTGATTATCCAGAAG | 6092 |
| Waxy starch | CGACTATAACTGCCTCACACTTTGTTTCTCATGTCTGTGGGGGTGC | 6093 |
| GBSS | CACTTCTGGAGAATGAAAAGTGGGGTTGGGTCAATTAGCCCTGAG | |
| Ipomoea batatas | GAGCCAAGCTGTGACTCACAATGGGTTGAG | |
| Ser22Term | CTCAACCCATTGTGAGTCACAGCTTGGCTCCTCAGGGCTAATTGA | 6094 |
| TCA-TGA | CCCAACCCCACTTTTCATTCTCCAGAAGTGGCACCCCCACAGACAT | |
| | GAGAAACAAAGTGTGAGGCAGTTATAGTCG | |
| | TGGAGAATGAAAAGTGG | 6095 |
| | CCACTTTTCATTCTCCA | 6096 |
| Waxy starch | ACTATAACTGCCTCACACTTTGTTTCTCATGTCTGTGGGGGTGCCA | 6097 |
| GBSS | CTTCTGGAGAATCATAAGTGGGGTTGGGTCAATTAGCCCTGAGGA | |
| Ipomoea batatas | GCCAAGCTGTGACTCACAATGGGTTGAGAC | |
| Lys23Term | GTCTCAACCCATTGTGAGTCACAGCTTGGCTCCTCAGGGCTAATT | 6098 |
| AAA-TAA | GACCCAACCCCACTTATGATTCTCCAGAAGTGGCACCCCCACAGA | |
| | CATGAGAAACAAAGTGTGAGGCAGTTATAGT | |
| | GAGAATCATAAGTGGGG | 6099 |
| | CCCCACTTATGATTCTC | 6100 |
| Waxy starch | CCTCACACTTTGTTTCTCATGTCTGTGGGGGTGCCACTTCTGGAGA | 6101 |
| GBSS | ATCAAAAGTGGGGTAGGGTCAATTAGCCCTGAGGAGCCAAGCTGT | |
| Ipomoea batatas | GACTCACAATGGGTTGAGACCTGTGAACAA | |
| Leu26Term | TTGTTCACAGGTCTCAACCCATTGTGAGTCACAGCTTGGCTCCTCA | 6102 |
| TTG-TAG | GGGCTAATTGACCCTACCCCACTTTTGATTCTCCAGAAGTGGCACC | |
| | CCCACAGACATGAGAAACAAAGTGTGAGG | |
| | AGTGGGGTAGGGTCAAT | 6103 |
| | ATTGACCCTACCCCACT | 6104 |
| Waxy starch | CATCGGCGATTGTTGCTCCTTACTGCTCTCTCACAGAATGGCAACG | 6105 |
| GBSS | GTGACGGGGTCTTAGGTGGTGTCGAGAAGCGCGTGCTTCAATTCC | |
| Astragalus | CAGGGAAGAACAGAAGCCAAAGTGAATTCA | |
| membranaeus | TGAATTCACTTTGGCTTCTGTTCTTCCCTGGGAATTGAAGCACGCG | 6106 |
| Tyr8Term | CTTCTCGACACCACCTAAGACCCCGTCACCGTTGCCATTCTGTGA | |
| TAT-TAG | GAGAGCAGTAAGGAGCAACAATCGCCGATG | |
| | GGGTCTTAGGTGGTGTC | 6107 |
| | GACACCACCTAAGACCC | 6108 |
| Waxy starch | ATTGTTGCTCCTTACTGCTCTCTCACAGAATGGCAACGGTGACGG | 6109 |
| GBSS | GGTCTTATGTGGTGTAGAGAAGCGCGTGCTTCAATTCCCAGGGAA | |
| Astragalus | GAACAGAAGCCAAAGTGAATTCACCTCAGAA | |
| membranaeus | TTCTGAGGTGAATTCACTTTGGCTTCTGTTCTTCCCTGGGAATTGA | 6110 |
| Ser11Term | AGCACGCGCTTCTCTACACCACATAAGACCCCGTCACCGTTGCCA | |
| TCG-TAG | TTCTGTGAGAGAGCAGTAAGGAGCAACAAT | |
| | TGTGGTGTAGAGAAGCG | 6111 |
| | CGCTTCTCTACACCACA | 6112 |
| Waxy starch | TGTTGCTCCTTACTGCTCTCTCACAGAATGGCAACGGTGACGGGG | 6113 |
| GBSS | TCTTATGTGGTGTCGTGAAGCGCGTGCTTCAATTCCCAGGGAAGA | |
| Astragalus | ACAGAAGCCAAAGTGAATTCACCTCAGAAGA | |
| membranaeus | TCTTCTGAGGTGAATTCACTTTGGCTTCTGTTCTTCCCTGGGAATT | 6114 |
| Arg12Term | GAAGCACGCGCTTCACGACACCACATAAGACCCCGTCACCGTTGC | |
| AGA-TGA | CATTCTGTGAGAGAGCAGTAAGGAGCAACA | |
| | TGGTGTCGTGAAGCGCG | 6115 |
| | CGCGCTTCACGACACCA | 6116 |
| Waxy starch | ACTGCTCTCTCACAGAATGGCAACGGTGACGGGGTCTTATGTGGT | 6117 |
| GBSS | GTCGAGAAGCGCGTGATTCAATTCCCAGGGAAGAACAGAAGCCAA | |
| Astragalus | AGTGAATTCACCTCAGAAGATAAATCTCAAT | |
| membranaeus | ATTGAGATTTATCTTCTGAGGTGAATTCACTTTGGCTTCTGTTCTTC | 6118 |
| Cys15Term | CCTGGGAATTGAATCACGCGCTTCTCGACACCACATAAGACCCCG | |
| TGC-TGA | TCACCGTTGCCATTCTGTGAGAGAGCAGT | |
| | AGCGCGTGATTCAATTC | 6119 |
| | GAATTGAATCACGCGCT | 6120 |
| Waxy starch | CACAGAATGGCAACGGTGACGGGGTCTTATGTGGTGTCGAGAAG | 6121 |
| GBSS | CGCGTGCTTCAATTCCTAGGGAAGAACAGAAGCCAAAGTGAATTC | |
| Astragalus | ACCTCAGAAGATAAATCTCAATAGCCAAGCAT | |
| membranaeus | ATGCTTGGCTATTGAGATTTATCTTCTGAGGTGAATTCACTTTGGCT | 6122 |
| Gln19Term | TCTGTTCTTCCCTAGGAATTGAAGCACGCGCTTCTCGACACCACAT | |
| CAG-TAG | AAGACCCCGTCACCGTTGCCATTCTGTG | |
| | TCAATTCCTAGGGAAGA | 6123 |
| | TCTTCCCTAGGAATTGA | 6124 |
| Waxy starch | TGTAGCTTGGTAGATTCCCCTTTTTGTAGACCACACATCACATGGC | 6125 |
| GBSS | AAGCATCACAGCTTGACACCACTTTGTGTCAAGAAGCCAAACTTCA | |
| Solanum tuberosum | CTAGACACCAAATCAACCTTGTCACAGAT | |
| Ser7Term | ATCTGTGACAAGGTTGATTTGGTGTCTAGTGAAGTTTGGCTTCTTG | 6126 |
| TCA-TGA | ACACAAAGTGGTGTCAAGCTGTGATGCTTGCCATGTGATGTGTGG | |
| | TCTACAAAAAGGGGAATCTACCAAGCTACA | |
| | CACAGCTTGACACCACT | 6127 |
| | AGTGGTGTCAAGCTGTG | 6128 |
| Waxy starch | TCCCCTTTTTGTAGACCACACATCACATGGCAAGCATCACAGCTTC | 6129 |
| GBSS | ACACCACTTTGTGTGAAGAAGCCAAACTTCACTAGACACCAAATCA | |
| Solanum tuberosum | ACCTTGTCACAGATAGGACTCAGGAACCA | |
| Ser12Term | TGGTTCCTGAGTCCTATCTGTGACAAGGTTGATTTGGTGTCTAGTG | 6130 |
| TCA-TGA | AAGTTTGGCTTCTTCACACAAAGTGGTGTGAAGCTGTGATGCTTGC | |
| | CATGTGATGTGTGGTCTACAAAAAGGGGA | |
| | CTTTGTGTGAAGAAGCC | 6131 |
| | GGCTTCTTCACACAAAG | 6132 |
| Waxy starch | CCCTTTTTGTAGACCACACATCACATGGCAAGCATCACAGCTTCAC | 6133 |
| GBSS | ACCACTTTGTGTCATGAAGCCAAACTTCACTAGACACCAAATCAAC | |
| Solanum tuberosum | CTTGTCACAGATAGGACTCAGGAACCATA | |
| Arg13Term | TATGGTTCCTGAGTCCTATCTGTGACAAGGTTGATTTGGTGTCTAG | 6134 |
| AGA-TGA | TGAAGTTTGGCTTCATGACACAAAGTGGTGTGAAGCTGTGATGCTT | |
| | GCCATGTGATGTGTGGTCTACAAAAAGGG | |
| | TTGTGTCATGAAGCCAA | 6135 |
| | TTGGCTTCATGACACAA | 6136 |
| Waxy starch | TTGTAGACCACACATCACATGGCAAGCATCACAGCTTCACACCACT | 6137 |
| GBSS | TTGTGTCAAGAAGCTAAACTTCACTAGACACCAAATCAACCTTGTC | |
| Solanum tuberosum | ACAGATAGGACTCAGGAACCATACTCTGA | |
| Gln15Term | TCAGAGTATGGTTCCTGAGTCCTATCTGTGACAAGGTTGATTTGGT | 6138 |
| CAA-TAA | GTCTAGTGAAGTTTAGCTTCTTGACACAAAGTGGTGTGAAGCTGTG | |
| | ATGCTTGCCATGTGATGTGTGGTCTACAA | |
| | CAAGAAGCTAAACTTCA | 6139 |
| | TGAAGTTTAGCTTCTTG | 6140 |
| Waxy starch | CCACACATCACATGGCAAGCATCACAGCTTCACACCACTTTGTGTC | 6141 |
| GBSS | AAGAAGCCAAACTTGACTAGACACCAAATCAACCTTGTCACAGATA | |
| Solanum tuberosum | GGACTCAGGAACCATACTCTGACTCACAA | |
| Ser17Term | TTGTGAGTCAGAGTATGGTTCCTGAGTCCTATCTGTGACAAGGTTG | 6142 |
| TCA-TGA | ATTTGGTGTCTAGTCAAGTTTGGCTTCTTGACACAAAGTGGTGTGA | |
| | AGCTGTGATGCTTGCCATGTGATGTGTGG | |
| | CCAAACTTGACTAGACA | 6143 |
| | TGTCTAGTCAAGTTTGG | 6144 |
| Waxy starch | GTCGATCACTCTTCTCTCACCGCCGAAACAGATTTTGACACAATAAA | 6145 |
| GBSS | TGGCAACAATAACGTGATCTTCAATGCCGACGAGAACCGCGTGCT | |
| Pisum sativum | TCAATTACCAAGGAAGATCAGCAGAGTCTA | |
| Gly6Term | TAGACTCTGCTGATCTTCCTTGGTAATTGAAGCACGCGGTTCTCGT 6146 | |
| GGA-TGA | CGGCATTGAAGATCACGTTATTGTTGCCATTTTTGTGTCAAAATCT | |
| | GTTTCGGCGGTGAGAGAAGAGTGATCGAC | |
| | CAATAACGTGATCTTCA | 6147 |
| | TGAAGATCACGTTATTG | 6148 |
| Waxy starch | ACTCTTCTCTCACCGCCGAAACAGATTTTGACACAAAAATGGCAAC | 6149 |
| GBSS | AATAACGGGATCTTGAATGCCGACGAGAACCGCGTGCTTCAATTA | |
| Pisum sativum | CCAAGGAAGATCAGCAGAGTCTAAACTGAA | |
| Ser8Term | TTCAGTTTAGACTCTGCTGATCTTCCTTGGTAATTGAAGCACGCGG | 6150 |
| TCA-TGA | TTCTCGTCGGCATTCAAGATCCCGTTATTGTTGCCATTTTTGTGTCA | |
| | AAATCTGTTTCGGCGGTGAGAGAAGAGT | |
| | GGGATCTTGAATGCCGA | 6151 |
| | TCGGCATTCAAGATCCC | 6152 |
| Waxy starch | ACCGCCGAAACAGATTTTGACACAAAAATGGCAACAATAACGGGA | 6153 |
| GBSS | TCTTCAATGCCGACGTGAACCGCGTGCTTCAATTACCAAGGAAGA | |
| Pisum sativum | TCAGCAGAGTCTAAACTGAATTTGCCTCAGA | |
| Arg12Term | TCTGAGGCAAATTCAGTTTAGACTCTGCTGATCTTCCTTGGTAATT | 6154 |
| AGA-TGA | GAAGCACGCGGTTCACGTCGGCATTGAAGATCCCGTTATTGTTGC | |
| | CATTTTTGTGTCAAAATCTGTTTCGGCGGT | |
| | TGCCGACGTGAACCGCG | 6155 |
| | CGCGGTTCACGTCGGCA | 6156 |
| Waxy starch | AGATTTTGACACAAAAATGGCAACAATAACGGGATCTTCAATGCCG | 6157 |
| GBSS | ACGAGAACCGCGTGATTCAATTACCAAGGAAGATCAGCAGAGTCT | |
| Pisum sativum | AAACTGAATTTGCCTCAGATACACTTCAAT | |
| Cys18Term | ATTGAAGTGTATCTGAGGCAAATTCAGTTTAGACTCTGCTGATCTT | 6158 |
| TGC-TGA | CCTTGGTAATTGAATCACGCGGTTCTCGTCGGCATTGAAGATCCC | |
| | GTTATTGTTGCCATTTTTGTGTCAAAATCT | |
| | ACCGCGTGATTCAATTA | 6159 |
| | TAATTGAATCACGCGGT | 6160 |
| Waxy starch | CACAAAAATGGCAACAATAACGGGATCTTCAATGCCGACGAGAAC | 6161 |
| GBSS | CGCGTGCTTCAATTAGCAAGGAAGATCAGCAGAGTCTAAACTGAA | |
| Pisum sativum | TTTGCCTCAGATACACTTCAATAACAACCAA | |
| Tyr18Term | TTGGTTGTTATTGAAGTGTATCTGAGGCAAATTCAGTTTAGACTCT | 6162 |
| TAC-TAG | GCTGATCTTCCTTGCTAATTGAAGCACGCGGTTCTCGTCGGCATTG | |
| | AAGATCCCGTTATTGTTGCCATTTTTGTG | |
| | TTCAATTAGCAAGGAAG | 6163 |
| | CTTCCTTGCTAATTGAA | 6164 |
| Waxy starch | TCTACACCGGAGAGAGCACCATGGCAACTGTAATAGCTGCACATT | 6165 |
| GBSS | TCGTTTCCAGGAGCTGACACTTGAGCATCCATGCATTAGAGACTAA | |
| Manihot esculenta | GGCTAATAATTTGTCTCACACTGGACCCTG | |
| Ser14Term | CAGGGTCCAGTGTGAGACAAATTATTAGCCTTAGTCTCTAATGCAT | 6166 |
| TCA-TGA | GGATGCTCAAGTGTCAGCTCCTGGAAACGAAATGTGCAGCTATTA | |
| | CAGTTGCCATGGTGCTCTCTCCGGTGTAGA | |
| | CAGGAGCTGACACTTGA | 6167 |
| | TCAAGTGTCAGCTCCTG | 6168 |
| Waxy starch | CCGGAGAGAGCACCATGGCAACTGTAATAGCTGCACATTTCGTTT | 6169 |
| GBSS | CCAGGAGCTCACACTAGAGCATCCATGCATTAGAGACTAAGGCTA | |
| Manihot esculenta | ATAATTTGTCTCACACTGGACCCTGGACCCA | |
| Leu16Term | TGGGTCCAGGGTCCAGTGTGAGACAAATTATTAGCCTTAGTCTCTA | 6170 |
| TTG-TAG | ATGCATGGATGCTCTAGTGTGAGCTCCTGGAAACGAAATGTGCAG | |
| | CTATTACAGTTGCCATGGTGCTCTCTCCGG | |
| | CTCACACTAGAGCATCC | 6171 |
| | GGATGCTCTAGTGTGAG | 6172 |
| Waxy starch | TGGCAACTGTAATAGCTGCACATTTCGTTTCCAGGAGCTCACACTT | 6173 |
| GBSS | GAGCATCCATGCATGAGAGACTAAGGCTT4ATAATTTGTCTCACACT | |
| Manihot esculenta | GGACCCTGGACCCAAACTATCACTCCCAA | |
| Leu21Term | TTGGGAGTGATAGTTTGGGTCCAGGGTCCAGTGTGAGACAAATTA | 6174 |
| TTA-TGA | TTAGCCTTAGTCTCTCATGCATGGATGCTCAAGTGTGAGCTCCTGG | |
| | AAACGAAATGTGCAGCTATTACAGTTGCCA | |
| | CCATGCATGAGAGACTA | 6175 |
| | TAGTCTCTCATGCATGG | 6176 |
| Waxy starch | GCAACTGTAATAGCTGCACATTTCGTTTCCAGGAGCTCACACTTGA | 6177 |
| GBSS | GCATCCATGCATTATAGACTAAGGCTAATAATTTGTCTCACACTGG | |
| Manihot esculenta | ACCCTGGACCCAAACTATCACTCCCAATG | |
| Glu22Term | CATTGGGAGTGATAGTTTGGGTCCAGGGTCCAGTGTGAGACAAAT | 6178 |
| GAG-TAG | TATTAGCCTTAGTCTATAATGCATGGATGCTCAAGTGTGAGCTCCT | |
| | GGAAACGAAATGTGCAGCTATTACAGTTGC | |
| | ATGCATTATAGACTAAG | 6179 |
| | CTTAGTCTATAATGCAT | 6180 |
| Waxy starch | GTAATAGCTGCACATTTCGTTTCCAGGAGCTCACACTTGAGCATCC | 6181 |
| GBSS | ATGCATTAGAGACTTAGGCTAATAATTTGTCTCACACTGGACCCTG | |
| Manihot esculenta | GACCCAAACTATCACTCCCAATGGTTTAA | |
| Lys24Term | TTAAACCATTGGGAGTGATAGTTTGGGTCCAGGGTCCAGTGTGAG | 6182 |
| AAG-TAG | ACAAATTATTAGCCTAAGTCTCTAATGCATGGATGCTCAAGTGTGA | |
| | GCTCCTGGAAACGAAATGTGCAGCTATTAC | |
| | TAGAGACTTAGGCTAAT | 6183 |
| | ATTAGCCTAAGTCTCTA | 6184 |
| Waxy starch | ACAACTCCTCCGTCACCGGTATAAGCATGGCAACGGTATCGATGG | 6185 |
| GBSS | CATCGTGCGTGGCGTGAAAAGGCGCGTGGAGTACAGAGACAAAA | |
| Phaseolus vulgaris | GTGAAATCTTCGGGTCAGATGAGCCTGAACCG | |
| Ser12Term | CGGTTCAGGCTCATCTGACCCGAAGATTTCACTTTTGTCTCTGTAC | 6186 |
| TCA-TGA | TCCACGCGCCTTTTCACGCCACGCACGATGCCATCGATACCGTTG | |
| | CCATGCTTATACCGGTGACGGAGGAGTTGT | |
| | CGTGGCGTGAAAAGGCG | 6187 |
| | CGCCTTTTCACGCCACG | 6188 |
| Waxy starch | CACCGGTATAAGCATGGCAACGGTATCGATGGCATCGTGCGTGGC | 6189 |
| GBSS | GTCAAAAGGCGCGTGAAGTACAGAGACAAAAGTGAAATCTTCGGG | |
| Phaseolus vulgaris | TCAGATGAGCCTGAACCGTCATGAATTGAAA | |
| Trp16Term | TTTCAATTCATGACGGTTCAGGCTCATCTGACCCGAAGATTTCACT | 6190 |
| TGG-TGA | TTTGTCTCTGTACTTCACGCGCCTTTTGACGCCACGCACGATGCCA | |
| | TCGATACCGTTGCCATGCTTATACCGGTG | |
| | GGCGCGTGAAGTACAGA | 6191 |
| | TCTGTACTTCACGCGCC | 6192 |
| Waxy starch | ATAAGCATGGCAACGGTATCGATGGCATCGTGCGTGGCGTCAAAA | 6193 |
| GBSS | GGCGCGTGGAGTACATAGACAAAAGTGAAATCTTCGGGTCAGATG | |
| Phaseolus vulgaris | AGCCTGAACCGTCATGAATTGAAATACGATG | |
| Glu19Term | CATCGTATTTCAATTCATGACGGTTCAGGCTCATCTGACCCGAAGA | 6194 |
| GAG-TAG | TTTCACTTTTGTCTATGTACTCCACGCGCCTTTTGACGCCACGCAC | |
| | GATGCCATCGATACCGTTGCCATGCTTAT | |
| | GGAGTACATAGACAAAA | 6195 |
| | TTTTGTCTATGTACTCC | 6196 |
| Waxy starch | ATGGCAACGGTATCGATGGCATCGTGCGTGGCGTCAAAAGGCGC | 6197 |
| GBSS | GTGGAGTACAGAGACATAAGTGAAATCTTCGGGTCAGATGAGCCT | |
| Phaseolus vulgaris | GAACCGTCATGAATTGAAATACGATGGGTTGA | |
| Lys21Term | TCAACCCATCGTATTTCAATTCATGACGGTTCAGGCTCATCTGACC | 6198 |
| AAA-TAA | CGAAGATTTCACTTATGTCTCTGTACTCCACGCGCCTTTTGACGCC | |
| | ACGCACGATGCCATCGATACCGTTGCCAT | |
| | CAGAGACATAAGTGAAA | 6199 |
| | TTTCACTTATGTCTCTG | 6200 |
| Waxy starch | ACGGTATCGATGGCATCGTGCGTGGCGTCAAAAGGCGCGTGGAG | 6201 |
| GBSS | TACAGAGACAAAAGTGTAATCTTCGGGTCAGATGAGCCTGAACCG | |
| Phaseolus vulgaris | TCATGAATTGAAATACGATGGGTTGAGATCTC | |
| Lys23Term | GAGATCTCAACCCATCGTATTTCAATTCATGACGGTTCAGGCTCAT | 6202 |
| AAA-TAA | CTGACCCGAAGATTACACTTTTGTCTCTGTACTCCACGCGCCTTTT | |
| | GACGCCACGCACGATGCCATCGATACCGT | |
| | CAAAAGTGTAATCTTCG | 6203 |
| | CGAAGATTACACTTTTG | 6204 |
| Waxy starch | GCGCCTAGCTCGAAAAGGTCGTCATTGAGAGGCTGCACCAATGG | 6205 |
| GBSS | GTTCCATTCCTAATTAGTGTTCTTATCAAACAAACAGTGTTGGTTCA | |
| Triticum aestivum | CTGAAACTGTCGCCTCACATCCAATTCCAG | |
| Tyr7Term | CTGGAATTGGATGTGAGGCGACAGTTTCAGTGAACCAACACTGTT | 6206 |
| TAT-TAG | TGTTTGATAAGAACACTAATTAGGAATGGAACCCATTGGTGCAGCC | |
| | TCTCAATGACGACCTTTTCGAGCTAGGCGC | |
| | CCTAATTAGTGTTCTTA | 6207 |
| | TAAGAACACTAATTAGG | 6208 |
| Waxy starch | CCTAGCTCGAAAAGGTCGTCATTGAGAGGCTGCACCAATGGGTTC | 6209 |
| GBSS | CATTCCTAATTATTGATCTTATCAAACAAACAGTGTTGGTTCACTGA | |
| Triticum aestivum | AACTGTCGCCTCACATCCAATTCCAGCAA | |
| Cys8Term | TTGCTGGAATTGGATGTGAGGCGACAGTTTCAGTGAACCAACACT | 6210 |
| TGT-TGA | GTTTGTTTGATAAGATCAATAATTAGGAATGGAACCCATTGGTGCA | |
| | GCCTCTCAATGACGACCTTTTCGAGCTAGG | |
| | AATTATTGATCTTATCA | 6211 |
| | TGATAAGATCAATAATT | 6212 |
| Waxy starch | TCGAAAAGGTCGTCATTGAGAGGCTGCACCAATGGGTTCCATTCC | 6213 |
| GBSS | TAATTATTGTTCTTAGCAAACAAACAGTGTTGGTTCACTGAAACTGT | |
| Triticum aestivum | CGCCTCACATCCAATTCCAGCAATCTTGT | |
| Tyr10Term | ACAAGATTGCTGGAATTGGATGTGAGGCGACAGTTTCAGTGAACC | 6214 |
| TAT-TAG | AACACTGTTTGTTTGCTAAGAACAATAATTAGGAATGGAACCCATT | |
| | GGTGCAGCCTCTCAATGACGACCTTTTCGA | |
| | TGTTCTTAGCAAACAAA | 6215 |
| | TTTGTTTGCTAAGAACA | 6216 |
| Waxy starch | CGAAAAGGTCGTCATTGAGAGGCTGCACCAATGGGTTCCATTCCT | 6217 |
| GBSS | AATTATTGTTCTTATTAAACAAACAGTGTTGGTTCACTGAAACTGTC | |
| Triticum aestivum | GCCTCACATCCAATTCCAGCAATCTTGTA | |
| Gln11Term | TACAAGATTGCTGGAATTGGATGTGAGGCGACAGTTTCAGTGAAC | 6218 |
| CAA-TAA | CAACACTGTTTGTTTAATAAGAACAATAATTAGGAATGGAACCCATT | |
| | GGTGCAGCCTCTCAATGACGACCTTTTCG | |
| | GTTCTTATTAAACAAAC | 6219 |
| | GTTTGTTTAATAAGAAC | 6220 |
| Waxy starch | AGGCTGCACCAATGGGTTCCATTCCTAATTATTGTTCTTATCAAACA | 6221 |
| GBSS | AACAGTGTTGGTTGACTGAAACTGTCGCCTCACATCCAATTCCAGC | |
| Triticum aestivum | AATCTTGTAACAATGAAGTTATGTTCCT | |
| Ser17Term | AGGAACATAACTTCATTGTTACAAGATTGCTGGAATTGGATGTGAG | 6222 |
| TCA-TGA | GCGACAGTTTCAGTCAACCAACACTGTTTGTTTGATAAGAACAATA | |
| | ATTAGGAATGGAACCCATTGGTGCAGCCT | |
| | TGTTGGTTGACTGAAAC | 6223 |
| | GTTTCAGTCAACCAACA | 6224 |
| Waxy starch | CAGCTCGCCACCTCCGGCACCGTCCTCGGCATCACCGACAGGTT | 6225 |
| GBSS | CCGGCGTGCAGGTTTCTAGGGCGTGAGGCCCCGGAGCCCGGCG | |
| Triticum aestivum | GATGCGGCTCTCGGCATGAGGACCGTCGGAGCTA | |
| Gln28Term | TAGCTCCGACGGTCCTCATGCCGAGAGCCGCATCCGCCGGGCTC | 6226 |
| CAG-TAG | CGGGGCCTCACGCCCTAGAAACCTGCACGCCGGAACCTGTCGGT | |
| | GATGCCGAGGACGGTGCCGGAGGTGGCGAGCTG | |
| | CAGGTTTCTAGGGCGTG | 6227 |
| | CACGCCCTAGAAACCTG | 6228 |
| Waxy starch | GGTTTCCAGGGCGTGAGGCCCCGGAGCCCGGCGGATGCGGCTCT | 6229 |
| GBSS | CGGCATGAGGACCGTCTGAGCTAGCGCCGCCCCAACGCAAAGCC | |
| Triticum aestivum | GGAAAGCGCACCGCGGGACCCGGCGGTGCCTCT | |
| Gly46Term | AGAGGCACCGCCGGGTCCCGCGGTGCGCTTTCCGGCTTTGCGTT | 6230 |
| GGA-TGA | GGGGCGGCGCTAGCTCAGACGGTCCTCATGCCGAGAGCCGCATC | |
| | CGCCGGGCTCCGGGGCCTCACGCCCTGGAAACC | |
| | GGACCGTCTGAGCTAGC | 6231 |
| | GCTAGCTCAGACGGTCC | 6232 |
| Waxy starch | CGGAGCCCGGCGGATGCGGCTCTCGGCATGAGGACCGTCGGAG | 6233 |
| GBSS | CTAGCGCCGCCCCAACGTAAAGCCGGAAAGCGCACCGCGGGACC | |
| Triticum aestivum | CGGCGGTGCCTCTCCATGGTGGTGCGCGCCACCG | |
| Gln53Term | CGGTGGCGCGCACCACCATGGAGAGGCACCGCCGGGTCCCGCG | 6234 |
| CAA-TAA | GTGCGCTTTCCGGCTTTACGTTGGGGCGGCGCTAGCTCCGACGG | |
| | TCCTCATGCCGAGAGCCGCATCCGCCGGGCTCCG | |
| | CCCCAACGTAAAGCCGG | 6235 |
| | CCGGCTTTACGTTGGGG | 6236 |
| Waxy starch | GCGGATGCGGCTCTCGGCATGAGGACCGTCGGAGCTAGCGCCGC | 6237 |
| GBSS | CCCAACGCAAAGCCGGTAAGCGCACCGCGGGACCCGGCGGTGC | |
| Triticum aestivum | CTCTCCATGGTGGTGCGCGCCACCGGCAGCGGCG | |
| Lys56Term | CGCCGCTGCCGGTGGCGCGCACCACCATGGAGAGGCACCGCCG | 6238 |
| AAA-TAA | GGTCCCGCGGTGCGCTTACCGGCTTTGCGTTGGGGCGGCGCTAG | |
| | CTCCGACGGTCCTCATGCCGAGAGCCGCATCCGC | |
| | AAAGCCGGTAAGCGCAC | 6239 |
| | TTGCGCTTACCGGCTTT | 6240 |
| Waxy starch | CTCTCCATGGTGGTGCGCGCCACCGGCAGCGGCGGCATGAACCT | 6241 |
| GBSS | CGTGTTCGTCGGCGCCTAGATGGCGCCCTGGAGCAAGACCGGCG | |
| Triticum aestivum | GCCTCGGCGACGTCCTCGGGGGCCTCCCCCCAG | |
| Glu85Term | CTGGGGGGAGGCCCCCGAGGACGTCGCCGAGGCCGCCGGTCTT | 6242 |
| GAG-TAG | GCTCCAGGGCGCCATCTAGGCGCCGACGAACACGAGGTTCATGC | |
| | CGCCGCTGCCGGTGGCGCGCACCACCATGGAGAG | |
| | TCGGCGCCTAGATGGCG | 6243 |
| | CGCCATCTAGGCGCCGA | 6244 |
| Waxy starch | GTCGTCTCTCGCTGCAGGTAGCCACACCCTGCGCGCGCGATGGC | 6245 |
| GBSS | GGCTCTGGTCACGTCGTAGCTCGCCACCTCCGGCACCGTCCTCG | |
| Triticum aestivum | GCATCACCGACAGGTTCCGGCGTGCAGGTTTTC | |
| Gln8Term | GAAAACCTGCACGCCGGAACCTGTCGGTGATGCCGAGGACGGTG | 6246 |
| CAG-TAG | CCGGAGGTGGCGAGCTACGACGTGACCAGAGCCGCCATCGCGC | |
| | GCGCAGGGTGTGGCTACCTGCAGCGAGAGACGAC | |
| | TCACGTCGTAGCTCGCC | 6247 |
| | GGCGAGCTACGACGTGA | 6248 |
| Waxy starch | CAGCTCGCCACCTCCGGCACCGTCCTCGGCATCACCGACAGGTT | 6249 |
| GBSS | CCGGCGTGCAGGTTTTTAGGGTGTGAGGCCCCGGAGCCCGGCAG | |
| Triticum aestivum | ATGCGCCGCTCGGCATGAGGACTACCGGAGCGA | |
| Gln28Term | TCGCTCCGGTAGTCCTCATGCCGAGCGGCGCATCTGCCGGGCTC | 6250 |
| CAG-TAG | CGGGGCCTCACACCCTAAAAACCTGCACGCCGGAACCTGTCGGT | |
| | GATGCCGAGGACGGTGCCGGAGGTGGCGAGCTG | |
| | CAGGTTTTTAGGGTGTG | 6251 |
| | CACACCCTAAAAACCTG | 6252 |
| Waxy starch | CCCCGGAGCCCGGCAGATGCGCCGCTCGGCATGAGGACTACCGG | 6253 |
| GBSS | AGCGAGCGCCGCCCCGTAGCAACAAAGCCGGAAAGCGCACCGCG | |
| Triticum aestivum | GGACCCGGCGGTGCCTCTCCATGGTGGTGCGCG | |
| Lys52Term | CGCGCACCACCATGGAGAGGCACCGCCGGGTCCCGCGGTGCGC | 6254 |
| AAG-TAG | TTTCCGGCTTTGTTGCTACGGGGCGGCGCTCGCTCCGGTAGTCCT | |
| | CATGCCGAGCGGCGCATCTGCCGGGCTCCGGGG | |
| | CCGCCCCGTAGCAACAA | 6255 |
| | TTGTTGCTACGGGGCGG | 6256 |
| Waxy starch | CGGAGCCCGGCAGATGCGCCGCTCGGCATGAGGACTACCGGAG | 6257 |
| GBSS | CGAGCGCCGCCCCGAAGTAACAAAGCCGGAAAGCGCACCGCGG | |
| Triticum aestivum | GACCCGGCGGTGCCTCTCCATGGTGGTGCGCGCCA | |
| Gln53Term | TGGCGCGCACCACCATGGAGAGGCACCGCCGGGTCCCGCGGTG | 6258 |
| CAA-TAA | CGCTTTCCGGCTTTGTTACTTCGGGGCGGCGCTCGCTCCGGTAGT | |
| | CCTCATGCCGAGCGGCGCATCTGCCGGGCTCCG | |
| | CCCCGAAGTAACAAAGC | 6259 |
| | GCTTTGTTACTTCGGGG | 6260 |
| Waxy starch | AGCCCGGCAGATGCGCCGCTCGGCATGAGGACTACCGGAGCGAG | 6261 |
| GBSS | CGCCGCCCCGAAGCAATAAAGCCGGAAAGCGCACCGCGGGACCC | |
| Triticum aestivum | GGCGGTGCCTCTCCATGGTGGTGCGCGCCACGG | |
| Gln54Term | CCGTGGCGCGCACCACCATGGAGAGGCACCGCCGGGTCCCGCG | 6262 |
| CAA-TAA | GTGCGCTTTCCGGCTTTATTGCTTCGGGGCGGCGCTCGCTCCGGT | |
| | AGTCCTCATGCCGAGCGGCGCATCTGCCGGGCT | |
| | CGAAGCAATAAAGCCGG | 6263 |
| | CCGGCTTTATTGCTTCG | 6264 |
| Waxy starch | CAGCTCGCCACCTCCGGCACCGTCCTCGGCATCACCGACAGGTT | 6265 |
| GBSS | CCGGCGTGCAGGTTTCTAGGGCGTGAGGCCCCGGAACCCGGCG | |
| Triticum durum | GATGCGGCCCTCGTCATGAGGACTATCGGAGCGA | |
| Gln28Term | TCGCTCCGATAGTCCTCATGACGAGGGCCGCATCCGCCGGGTTC | 6266 |
| CAG-TAG | CGGGGCCTCACGCCCTAGAAACCTGCACGCCGGAACCTGTCGGT | |
| | GATGCCGAGGACGGTGCCGGAGGTGGCGAGCTG | |
| | CAGGTTTCTAGGGCGTG | 6267 |
| | CACGCCCTAGAAACCTG | 6268 |
| Waxy starch | CCCCGGAACCCGGCGGATGCGGCCCTCGTCATGAGGACTATCGG | 6269 |
| GBSS | AGCGAGCGCCGCCCCGTAGCAAAGCCGGAAAGCGCACCGCGGG | |
| Triticum durum | AGCCGGCGGTGCCTCTCCATGGTGGTGCGCGCCA | |
| Lys52Term | TGGCGCGCACCACCATGGAGAGGCACCGCCGGCTCCCGCGGTG | 6270 |
| AAG-TAG | CGCTTTCCGGCTTTGCTACGGGGCGGCGCTCGCTCCGATAGTCCT | |
| | CATGACGAGGGCCGCATCCGCCGGGTTCCGGGG | |
| | CCGCCCCGTAGCAAAGC | 6271 |
| | GCTTTGCTACGGGGCGG | 6272 |
| Waxy starch | CGGAACCCGGCGGATGCGGCCCTCGTCATGAGGACTATCGGAGC | 6273 |
| GBSS | GAGCGCCGCCCCGAAGTAAAGCCGGAAAGCGCACCGCGGGAGC | |
| Triticum durum | CGGCGGTGCCTCTCCATGGTGGTGCGCGCCACGG | |
| Gln53Term | CCGTGGCGCGCACCACCATGGAGAGGCACCGCCGGCTCCCGCG | 6274 |
| CAA-TAA | GTGCGCTTTCCGGCTTTACTTCGGGGCGGCGCTCGCTCCGATAGT | |
| | CCTCATGACGAGGGCCGCATCCGCCGGGTTCCG | |
| | CCCCGAAGTAAAGCCGG | 6275 |
| | CCGGCTTTACTTCGGGG | 6276 |
| Waxy starch | GCGGATGCGGCCCTCGTCATGAGGACTATCGGAGCGAGCGCCGC | 6277 |
| GBSS | CCCGAAGCAAAGCCGGTAAGCGCACCGCGGGAGCCGGCGGTGC | |
| Triticum durum | CTCTCCATGGTGGTGCGCGCCACGGGCAGCGGCG | |
| Lys56Term | CGCCGCTGCCCGTGGCGCGCACCACCATGGAGAGGCACCGCCG | 6278 |
| AAA-TAA | GCTCCCGCGGTGCGCTTACCGGCTTTGCTTCGGGGCGGCGCTCG | |
| | CTCCGATAGTCCTCATGACGAGGGCCGCATCCGC | |
| | AAAGCCGGTAAGCGCAC | 6279 |
| | GTGCGCTTACCGGCTTT | 6280 |
| Waxy starch | TATCGGAGCGAGCGCCGCCCCGAAGCAAAGCCGGAAAGCGCACC | 6281 |
| GBSS | GCGGGAGCCGGCGGTGACTCTCCATGGTGGTGCGCGCCACGGG | |
| Triticum durum | CAGCGGCGGCATGAACCTCGTGTTCGTCGGCGCC | |
| Cys64Term | GGCGCCGACGAACACGAGGTTCATGCCGCCGCTGCCCGTGGCGC | 6282 |
| TGC-TGA | GCACCACCATGGAGAGTCACCGCCGGCTCCCGCGGTGCGCTTTC | |
| | CGGCTTTGCTTCGGGGCGGCGCTCGCTCCGATA | |
| | CGGCGGTGACTCTCCAT | 6283 |
| | ATGGAGAGTCACCGCCG | 6284 |
| Waxy starch | CAGCTCGCCACCTCCGGCACCGTCCTCGGCATCACCGACAGGTT | 6285 |
| GBSS | CCGGCGTGCAGGTTTTTAGGGTGTGAGGCCCCGGAGCCCGGCAG | |
| Triticum turgidum | ATGCGCCGCTCGGCATGAGGACTACCGGAGCGA | |
| Gln28Term | TCGCTCCGGTAGTCCTCATGCCGAGCGGCGCATCTGCCGGGCTC | 6286 |
| CAG-TAG | CGGGGCCTCACACCCTAAAAACCTGCACGCCGGAACCTGTCGGT | |
| | GATGCCGAGGACGGTGCCGGAGGTGGCGAGCTG | |
| | CAGGTTTTTAGGGTGTG | 6287 |
| | CACACCCTAAAAACCTG | 6288 |
| Waxy starch | CCCCGGAGCCCGGCAGATGCGCCGCTCGGCATGAGGACTACCGG | 6289 |
| GBSS | AGCGAGCGCCGCCCCGTAGCAACAAAGCCGGAAAGCGCACCGCG | |
| Triticum turgidum | GGACCCGGCGGTGCCTCTCCATGGTGGTGCGCG | |
| Lys52Term | CGCGCACCACCATGGAGAGGCACCGCCGGGTCCCGCGGTGCGC | 6290 |
| AAG-TAG | TTTCCGGCTTTGTTGCTACGGGGCGGCGCTCGCTCCGGTAGTCCT | |
| | CATGCCGAGCGGCGCATCTGCCGGGCTCCGGGG | |
| | CCGCCCCGTAGCAACAA | 6291 |
| | TTGTTGCTACGGGGCGG | 6292 |
| Waxy starch | CGGAGCCCGGCAGATGCGCCGCTCGGCATGAGGACTACCGGAG | 6293 |
| GBSS | CGAGCGCCGCCCCGAAGTAACAAAGCCGGAAAGCGCACCGCGG | |
| Triticum turgidum | GACCCGGCGGTGCCTCTCCATGGTGGTGCGCGCCA | |
| Gln53Term | TGGCGCGCACCACCATGGAGAGGCACCGCCGGGTCCCGCGGTG | 6294 |
| CAA-TAA | CGCTTTCCGGCTTTGTTACTTCGGGGCGGCGCTCGCTCCGGTAGT | |
| | CCTCATGCCGAGCGGCGCATCTGCCGGGCTCCG | |
| | CCCCGAAGTAACAAAGC | 6295 |
| | GCTTTGTTACTTCGGGG | 6296 |
| Waxy starch | AGCCCGGCAGATGCGCCGCTCGGCATGAGGACTACCGGAGCGAG | 6297 |
| GBSS | CGCCGCCCCGAAGCAATAAAGCCGGAAAGCGCACCGCGGGACCC | |
| Triticum turgidum | GGCGGTGCCTCTCCATGGTGGTGCGCGCCACGG | |
| Gln54Term | CCGTGGCGCGCACCACCATGGAGAGGCACCGCCGGGTCCCGCG | 6298 |
| CAA-TAA | GTGCGCTTTCCGGCTTTATTGCTTCGGGGCGGCGCTCGCTCCGGT | |
| | AGTCCTCATGCCGAGCGGCGCATCTGCCGGGCT | |
| | CGAAGCAATAAAGCCGG | 6299 |
| | CCGGCTTTATTGCTTCG | 6300 |
| Waxy starch | GATGCGCCGCTCGGCATGAGGACTACCGGAGCGAGCGCCGCCCC | 6301 |
| GBSS | GAAGCAACAAAGCCGGTAAGCGCACCGCGGGACCCGGCGGTGC | |
| Triticum turgidum | CTCTCCATGGTGGTGCGCGCCACGGGCAGCGCCG | |
| Lys57Term | CGGCGCTGCCCGTGGCGCGCACCACCATGGAGAGGCACCGCCG | 6302 |
| AAA-TAA | GGTCCCGCGGTGCGCTTACCGGCTTTGTTGCTTCGGGGCGGCGC | |
| | TCGCTCCGGTAGTCCTCATGCCGAGCGGCGCATC | |
| | AAAGCCGGTAAGCGCAC | 6303 |
| | GTGCGCTTACCGGCTTT | 6304 |
| Waxy starch | CAGCTCGCCACCTCCGCCACCGTCCTCGGCATCACCGACAGGTTC | 6305 |
| GBSS | CGCCATGCAGGTTTCTAGGGCGTGAGGCCCCGGAGCCCGGCAGA | |
| Aegilops speltoides | TGCGCCGCTCGGCATGAGGACTGTCGGAGCGA | |
| Gln28Term | TCGCTCCGACAGTCCTCATGCCGAGCGGCGCATCTGCCGGGCTC | 6306 |
| CAG-TAG | CGGGGCCTCACGCCCTAGAAACCTGCATGGCGGAACCTGTCGGT | |
| | GATGCCGAGGACGGTGGCGGAGGTGGCGAGCTG | |
| | CAGGTTTCTAGGGCGTG | 6307 |
| | CACGCCCTAGAAACCTG | 6308 |
| Waxy starch | GGTTTCCAGGGCGTGAGGCCCCGGAGCCCGGCAGATGCGCCGCT | 6309 |
| GBSS | CGGCATGAGGACTGTCTGAGCGAGCGCCGCCCCGAAGCAACAAA | |
| Aegilops speltoides | GCCGGAAAGCGCACCGCGGGACCCGGCGGTGCC | |
| Gly46Term | GGCACCGCCGGGTCCCGCGGTGCGCTTTCCGGCTTTGTTGCTTC | 6310 |
| GGA-TGA | GGGGCGGCGCTCGCTCAGACAGTCCTCATGCCGAGCGGCGCATC | |
| | TGCCGGGCTCCGGGGCCTCACGCCCTGGAAACC | |
| | GGACTGTCTGAGCGAGC | 6311 |
| | GCTCGCTCAGACAGTCC | 6312 |
| Waxy starch | CCCCGGAGCCCGGCAGATGCGCCGCTCGGCATGAGGACTGTCGG | 6313 |
| GBSS | AGCGAGCGCCGCCCCGTAGCAACAAAGCCGGAAAGCGCACCGCG | |
| Aegilops speltoides | GGACCCGGCGGTGCCTCTCGATGGTGGTGCGCG | |
| Lys52Term | CGCGCACCACCATCGAGAGGCACCGCCGGGTCCCGCGGTGCGCT | 6314 |
| AAG-TAG | TTCCGGCTTTGTTGCTACGGGGCGGCGCTCGCTCCGACAGTCCTC | |
| | ATGCCGAGCGGCGCATCTGCCGGGCTCCGGGG | |
| | CCGCCCCGTAGCAACAA | 6315 |
| | TTGTTGCTACGGGGCGG | 6316 |
| Waxy starch | CGGAGCCCGGCAGATGCGCCGCTCGGCATGAGGACTGTCGGAG | 6317 |
| GBSS | CGAGCGCCGCCCCGAAGTAACAAAGCCGGAAAGCGCACCGCGG | |
| Aegilops speltoides | GACCCGGCGGTGCCTCTCGATGGTGGTGCGCGCCA | |
| Gln53Term | TGGCGCGCACCACCATCGAGAGGCACCGCCGGGTCCCGCGGTG | 6318 |
| CAA-TAA | CGCTTTCCGGCTTTGTTACTTCGGGGCGGCGCTCGCTCCGACAGT | |
| | CCTCATGCCGAGCGGCGCATCTGCCGGGCTCCG | |
| | CCCCGAAGTAACAAAGC | 6319 |
| | GCTTTGTTACTTCGGGG | 6320 |
| Waxy starch | AGCCCGGCAGATGCGCCGCTCGGCATGAGGACTGTCGGAGCGAG | 6321 |
| GBSS | CGCCGCCCCGAAGCAATAAAGCCGGAAAGCGCACCGCGGGACCC | |
| Aegilops speltoides | GGCGGTGCCTCTCGATGGTGGTGCGCGCCACCG | |
| Gln54Term | CGGTGGCGCGCACCACCATCGAGAGGCACCGCCGGGTCCCGCG | 6322 |
| CAA-TAA | GTGCGCTTTCCGGCTTTATTGCTTCGGGGCGGCGCTCGCTCCGAC | |
| | AGTCCTCATGCCGAGCGGCGCATCTGCCGGGCT | |
| | CGAAGCAATAAAGCCGG | 6323 |
| | CCGGCTTTATTGCTTCG | 6324 |
| Waxy starch | AGTGCAGAGATCTTCCACAGCAACAGCTAGACAACCACCATGTCG | 6325 |
| GBSS | GCTCTCACCACGTCCTAGCTCGCCACCTCGGCCACCGGCTTCGG | |
| Oryza glaberrima | CATCGCTGACAGGTCGGCGCCGTCGTCGCTGC | |
| Gln8Term | GCAGCGACGACGGCGCCGACCTGTCAGCGATGCCGAAGCCGGT | 6326 |
| CAG-TAG | GGCCGAGGTGGCGAGCTAGGACGTGGTGAGAGCCGACATGGTG | |
| | GTTGTCTAGCTGTTGCTGTGGAAGATCTCTGCACT | |
| | CCACGTCCTAGCTCGCC | 6327 |
| | GGCGAGCTAGGACGTGG | 6328 |
| Waxy starch | TCCACAGCAACAGCTAGACAACCACCATGTCGGCTCTCACCACGT | 6329 |
| GBSS | CCCAGCTCGCCACCTAGGCCACCGGCTTCGGCATCGCTGACAGG | |
| Oryza glaberrima | TCGGCGCCGTCGTCGCTGCTCCGCCACGGGTT | |
| Ser12Term | AACCCGTGGCGGAGCAGCGACGACGGCGCCGACCTGTCAGCGAT | 6330 |
| TCG-TAG | GCCGAAGCCGGTGGCCTAGGTGGCGAGCTGGGACGTGGTGAGA | |
| | GCCGACATGGTGGTTGTCTAGCTGTTGCTGTGGA | |
| | CGCCACCTAGGCCACCG | 6331 |
| | CGGTGGCCTAGGTGGCG | 6332 |
| Waxy starch | CGGCTCTCACCACGTCCCAGCTCGCCACCTCGGCCACCGGCTTC | 6333 |
| GBSS | GGCATCGCTGACAGGTAGGCGCCGTCGTCGCTGCTCCGCCACGG | |
| Oryza glaberrima | GTTCCAGGGCCTCAAGCCCCGCAGCCCCGCCGG | |
| Ser22Term | CCGGCGGGGCTGCGGGGCTTGAGGCCCTGGAACCCGTGGCGGA | 6334 |
| TCG-TAG | GCAGCGACGACGGCGCCTACCTGTCAGCGATGCCGAAGCCGGTG | |
| | GCCGAGGTGGCGAGCTGGGACGTGGTGAGAGCCG | |
| | TGACAGGTAGGCGCCGT | 6335 |
| | ACGGCGCCTACCTGTCA | 6336 |
| Waxy starch | CCACGTCCCAGCTCGCCACCTCGGCCACCGGCTTCGGCATCGCT | 6337 |
| GBSS | GACAGGTCGGCGCCGTAGTCGCTGCTCCGCCACGGGTTCCAGGG | |
| Oryza glaberrima | CCTCAAGCCCCGCAGCCCCGCCGGCGGCGACGC | |
| Ser25Term | GCGTCGCCGCCGGCGGGGCTGCGGGGCTTGAGGCCCTGGAACC | 6338 |
| TCG-TAG | CGTGGCGGAGCAGCGACTACGGCGCCGACCTGTCAGCGATGCCG | |
| | AAGCCGGTGGCCGAGGTGGCGAGCTGGGACGTGG | |
| | GGCGCCGTAGTCGCTGC | 6339 |
| | GCAGCGACTACGGCGCC | 6340 |
| Waxy starch | CGTCCCAGCTCGCCACCTCGGCCACCGGCTTCGGCATCGCTGAC | 6341 |
| GBSS | AGGTCGGCGCCGTCGTAGCTGCTCCGCCACGGGTTCCAGGGCCT | |
| Oryza glaberrima | CAAGCCCCGCAGCCCCGCCGGCGGCGACGCGAC | |
| Ser26Term | GTCGCGTCGCCGCCGGCGGGGCTGCGGGGCTTGAGGCCCTGGA | 6342 |
| TCG-TAG | ACCCGTGGCGGAGCAGCTACGACGGCGCCGACCTGTCAGCGATG | |
| | CCGAAGCCGGTGGCCGAGGTGGCGAGCTGGGACG | |
| | GCCGTCGTAGCTGCTCC | 6343 |
| | GGAGCAGCTACGACGGC | 6344 |
| Waxy starch | TCCACAGCAAGAGCTAAACAGCCGACCGTGTGCACCACCATGTCG | 6345 |
| GBSS | GCTCTCACCACGTCCTAGCTCGCCACCTCGGCCACCGGCTTCGG | |
| Oryza sativa | CATCGCCGACAGGTCGGCGCCGTCGTCGCTGC | |
| Gln8Term | GCAGCGACGACGGCGCCGACCTGTCGGCGATGCCGAAGCCGGT | 6346 |
| GAG-TAG | GGCCGAGGTGGCGAGCTAGGACGTGGTGAGAGCCGACATGGTG | |
| | GTGCACACGGTCGGCTGTTTAGCTCTTGCTGTGGA | |
| | CCACGTCCTAGCTCGCC | 6347 |
| | GGCGAGCTAGGACGTGG | 6348 |
| Waxy starch | CTAAACAGCCGACCGTGTGCACCACCATGTCGGCTCTCACCACGT | 6349 |
| GBSS | CCCAGCTCGCCACCTAGGCCACCGGCTTCGGCATCGCCGACAGG | |
| Oryza sativa | TCGGCGCCGTCGTCGCTGCTTCGCCACGGGTT | |
| Ser12Term | AACCCGTGGCGAAGCAGCGACGACGGCGCCGACCTGTCGGCGAT | 6350 |
| TCG-TAG | GCCGAAGCCGGTGGCCTAGGTGGCGAGCTGGGACGTGGTGAGA | |
| | GCCGACATGGTGGTGCACACGGTCGGCTGTTTAG | |
| | CGCCACCTAGGCCACCG | 6351 |
| | CGGTGGCCTAGGTGGCG | 6352 |
| Waxy starch | CGGCTCTCACCACGTCCCAGCTCGCCACCTCGGCCACCGGCTTC | 6353 |
| GBSS | GGCATCGCCGACAGGTAGGCGCCGTCGTCGCTGCTTCGCCACGG | |
| Oryza sativa | GTTCCAGGGCCTCAAGCCCCGTAGCCCAGCCGG | |
| Ser22Term | CCGGCTGGGCTACGGGGCTTGAGGCCCTGGAACCCGTGGCGAA | 6354 |
| TCG-TAG | GCAGCGACGACGGCGCCTACCTGTCGGCGATGCCGAAGCCGGTG | |
| | GCCGAGGTGGCGAGCTGGGACGTGGTGAGAGCCG | |
| | CGACAGGTAGGCGCCGT | 6355 |
| | ACGGCGCCTACCTGTCG | 6356 |
| Waxy starch | CCACGTCCCAGCTCGCCACCTCGGCCACCGGCTTCGGCATCGCC | 6357 |
| GBSS | GACAGGTCGGCGCCGTAGTCGCTGCTTCGCCACGGGTTCCAGGG | |
| Oryza sativa | CCTCAAGCCCCGTAGCCCAGCCGGCGGGGACGC | |
| Ser25Term | GCGTCCCCGCCGGCTGGGCTACGGGGCTTGAGGCCCTGGAACCC | 6358 |
| TCG-TAG | GTGGCGAAGCAGCGACTACGGCGCCGACCTGTCGGCGATGCCGA | |
| | AGCCGGTGGCCGAGGTGGCGAGCTGGGACGTGG | |
| | GGCGCCGTAGTCGCTGC | 6359 |
| | GCAGCGACTACGGCGCC | 6360 |
| Waxy starch | CGTCCCAGCTCGCCACCTCGGCCACCGGCTTCGGCATCGCCGAC | 6361 |
| GBSS | AGGTCGGCGCCGTCGTAGCTGCTTCGCCACGGGTTCCAGGGCCT | |
| Oryza sativa | CAAGCCCCGTAGCCCAGCCGGCGGGGACGCATC | |
| Ser26Term | GATGCGTCCCCGCCGGCTGGGCTACGGGGCTTGAGGCCCTGGAA | 6362 |
| TCG-TAG | CCCGTGGCGAAGCAGCTACGACGGCGCCGACCTGTCGGCGATGC | |
| | CGAAGCCGGTGGCCGAGGTGGCGAGCTGGGACG | |
| | GCCGTCGTAGCTGCTTC | 6363 |
| | GAAGCAGCTACGACGGC | 6364 |
| Waxy starch | GTCTCTCACTGCAGGTAGCCACACCCTGTGCGCGGCGCCATGGC | 6365 |
| GBSS | GGCTCTGGCCACGTCCTAGCTCGCCACCTCCGGCACCGTCCTCG | |
| Hordeum vulgare | GCGTCACCGACAGATTCCGGCGTCCAGGTTTTC | |
| Gln8Term | GAAAACCTGGACGCCGGAATCTGTCGGTGACGCCGAGGACGGTG | 6366 |
| GAG-TAG | CCGGAGGTGGCGAGCTAGGACGTGGCCAGAGCCGCCATGGCGC | |
| | CGCGCACAGGGTGTGGCTACCTGCAGTGAGAGAC | |
| | CCACGTCCTAGCTCGCC | 6367 |
| | GGCGAGCTAGGACGTGG | 6368 |
| Waxy starch | ATGGCGGCTCTGGCCACGTCCCAGCTCGCCACCTCCGGCACCGT | 6369 |
| GBSS | CCTCGGCGTCACCGACTGATTCCGGCGTCCAGGTTTTCAGGGCCT | |
| Hordeum vulgare | CAGGCCCCGGAACCCGGCGGATGCGGCGCTTG | |
| Arg21Term | CAAGCGCCGCATCCGCCGGGTTCCGGGGCCTGAGGCCCTGAAAA | 6370 |
| AGA-TGA | CCTGGACGCCGGAATCAGTCGGTGACGCCGAGGACGGTGCCGG | |
| | AGGTGGCGAGCTGGGACGTGGCCAGAGCCGCCAT | |
| | TGACCGACTGATTCCGG | 6371 |
| | CCGGAATCAGTCGGTGA | 6372 |
| Waxy starch | CAGCTCGCCACCTCCGGCACCGTCCTCGGCGTCACCGACAGATT | 6373 |
| GBSS | CCGGCGTCCAGGTTTTTAGGGCCTCAGGCCCCGGAACCCGGCGG | |
| Hordeum vulgare | ATGCGGCGCTTGGTATGAGGACTATCGGAGCAA | |
| Gln28Term | TTGCTCCGATAGTCCTCATACCAAGCGCCGCATCCGCCGGGTTCC | 6374 |
| GAG-TAG | GGGGCCTGAGGCCCTAAAAACCTGGACGCCGGAATCTGTCGGTG | |
| | ACGCCGAGGACGGTGCCGGAGGTGGCGAGCTG | |
| | CAGGTTTTTAGGGCCTC | 6375 |
| | GAGGCCCTAAAAACCTG | 6376 |
| Waxy starch | GGTTTTGAGGGCCTCAGGCCCCGGAACCCGGCGGATGCGGCGCT | 6377 |
| GBSS | TGGTATGAGGACTATCTGAGCAAGCGCCGCCCCGAAGCAAAGCC | |
| Hordeum vulgare | GGAAAGCGCACCGCGGGAGCCGGCGGTGCCTCT | |
| Gly46Term | AGAGGCACCGCCGGCTCCCGCGGTGCGCTTTCCGGCTTTGCTTC | 6378 |
| GGA-TGA | GGGGCGGCGCTTGCTCAGATAGTCCTCATACCAAGCGCCGCATC | |
| | CGCCGGGTTCCGGGGCCTGAGGCCCTGAAAACC | |
| | GGACTATCTGAGCAAGC | 6379 |
| | GCTTGCTCAGATAGTCC | 6380 |
| Waxy starch | CCCCGGAACCCGGCGGATGCGGCGCTTGGTATGAGGACTATCGG | 6381 |
| GBSS | AGCAAGCGCCGCCCCGTAGCAAAGCCGGAAAGCGCACGGCGGG | |
| Hordeum vulgare | AGGCGGCGGTGCCTCTCCGTGGTGGTGAGCGCCA | |
| Lys52Term | TGGCGCTCACCACCACGGAGAGGCACCGCCGGCTGCCGCGGTGC | 6382 |
| AAG-TAG | GCTTTCCGGCTTTGCTACGGGGCGGCGCTTGCTCCGATAGTCCTC | |
| | ATACCAAGCGCCGCATCCGCCGGGTTCCGGGG | |
| | CCGCCCCGTAGCAAAGC | 6383 |
| | GCTTTGCTACGGGGCGG | 6384 |
| Waxy starch | ACGTCTTTTCTCTCTCTCCTACGCAGTGGATTAATCGGCATGGCGG | 6385 |
| GBSS | CTCTGGCCACGTCGTAGCTCGTCGCAACGCGCGCCGGCCTGGGC | |
| Zea mays | GTCCCGGACGCGTCCACGTTCCGCCGCGGCG | |
| Gln8Term | CGCCGCGGCGGAACGTGGACGCGTCCGGGACGCCCAGGCCGGC | 6386 |
| GAG-TAG | GCGCGTTGCGACGAGCTACGACGTGGCCAGAGGCGCCATGCCGA | |
| | TTAATCCACTGCGTAGGAGAGAGAGAAAAGACGT | |
| | GCACGTCGTAGCTCGTC | 6387 |
| | GACGAGCTACGACGTGG | 6388 |
| Waxy starch | GTCGCAACGCGCGCCGGCCTGGGCGTCCCGGACGCGTCCACGTT | 6389 |
| GBSS | CCGCCGCGGCGCCGCGTAGGGCCTGAGGGGGGCCCGGGCGTCG | |
| Zea mays | GCGGCGGCGGACACGCTCAGCATGCGGACCAGCG | |
| Gln30Term | CGCTGGTCCGCATGCTGAGCGTGTCCGCCGCCGCCGACGCCCGG | 6390 |
| GAG-TAG | GCCCCCCTCAGGCCCTACGCGGCGCCGCGGCGGAACGTGGACG | |
| | CGTCCGGGACGCCCAGGCCGGCGCGCGTTGCGAC | |
| | GCGCCGCGTAGGGCCTG | 6391 |
| | CAGGCCCTACGCGGCGC | 6392 |
| Waxy starch | TCCCGGACGCGTCCACGTTCCGCCGCGGCGCCGCGCAGGGCCT | 6393 |
| GBSS | GAGGGGGGCCCGGGCGTAGGCGGCGGCGGACACGCTCAGCATG | |
| Zea mays | CGGACCAGCGCGCGCGCGGCGCCCAGGCACCAGCA | |
| Ser38Term | TGCTGGTGCCTGGGCGCCGCGCGCGCGCTGGTCCGCATGCTGAG | 6394 |
| TCG-TAG | CGTGTCCGCCGCCGCCTACGCCCGGGCCCCCCTCAGGCCCTGCG | |
| | CGGCGCCGCGGCGGAACGTGGACGCGTCCGGGA | |
| | CCGGGCGTAGGCGGCGG | 6395 |
| | CCGCCGGCTACGCCCGG | 6396 |
| Waxy starch | GCGTCGGCGGCGGCGGACACGCTCAGCATGCGGACCAGCGCGC | 6397 |
| GBSS | GCGCGGCGCCCAGGCACTAGCAGCAGGCGCGCCGCGGGGGCAG | |
| Zea mays | GTTCCCGTCGCTCGTCGTGTGCGCCAGCGCCGGCA | |
| Ser57Term | TGCCGGCGCTGGCGCACACGACGAGCGACGGGAACCTGCCCCC | 6398 |
| GAG-TAG | GCGGCGCGCCTGGTGGTAGTGCCTGGGCGGGGCGCGCGGGCTG | |
| | GTCCGCATGGTGAGCGTGTCCGCGGCCGCCGACGC | |
| | CCAGGCACTAGCAGCAG | 6399 |
| | CTGCTGCTAGTGCCTGG | 6400 |
| Waxy starch | TCGGCGGCGGCGGACACGCTCAGCATGCGGACCAGCGCGCGCG | 6401 |
| GBSS | CGGCGCCCAGGCACCAGTAGCAGGCGCGCCGCGGGGGCAGGTT | |
| Zea mays | CCCGTCGCTCGTCGTGTGCGCCAGCGCCGGCATGA | |
| Gln58Term | TCATGCCGGCGCTGGCGCACACGACGAGCGACGGGAACCTGCCC | 6402 |
| GAG-TAG | CCGCGGCGCGCCTGCTACTGGTGCCTGGGCGCCGCGCGCGCGC | |
| | TGGTCCGCATGCTGAGCGTGTCCGCCGCCGCCGA | |
| | GGCACCAGTAGCAGGCG | 6403 |
| | CGCCTGCTACTGGTGCC | 6404 |

### EXAMPLE 32

### Altering Fatty Acid Content of Plants

Improved means to manipulate fatty acid compositions, from biosynthetic or natural plant sources, are needed. For example, oils containing reduced saturated fatty acids are desired for dietary reasons and oils containing increased saturated fatty acids are also needed as alternatives to current sources of highly saturated oil products, such as tropical oils or chemically hydrogenated oils. It would therefore be advantageous to influence directly the production and composition of fatty acids in crop plants.

Higher plants synthesize fatty acids, primarily palmitic, stearic and oleic acids, in the plastids (i.e., chloroplasts, proplastids, or other related organelles) as part of the Fatty Acid Synthase (FAS) complex. Fatty acid synthesis is the result of the three enzymatic activities: acyl-ACP elongase, acyl-ACP desaturase and acyl-ACP thioesterases specific for each of palmitoyl-, stearoyl- and oleoyl-ACP.

A variety of enzymes have been identified that influence the relative levels of saturated vs. unsaturated fatty acids in plants. For example, the enzymes stearoyl-acyl carrier protein (stearoyl-ACP) desaturase, oleoyl desaturase and linoleate desaturase produce unsaturated fatty acids from saturated precursors. Similarly, relative enzymatic activities of the various acyl-ACP thioesterases influences the relative acyl-chain composition of the resultant fatty acids. Consequently a reduction or an increase of the activity of these enzymes can alter the properties of oils produced in a plant. In fact, specific targeting of particular enzymatic activities can results in altered levels of particular fatty acids.

The attached tables disclose exemplary oligonucleotides base sequences which can be used to generate site-specific mutations in plant genes encoding proteins involved in fatty acid biosynthesis.

TABLE 40
Oligonucleotides to produce plants with reduced palmitate

| Phenotype, Gene, | | |
|---|---|---|
| Plant & Targeted | | SEQ ID |
| Alteration | Altering Oligos | NO: |
| Reduced palmitate | TTTGGTGGCAGTGTCTTTGAACGCTTCATCTCCTCGTCATGGTGGC | 6405 |
| Acyl-ACP-thioesterase | CACCTCTGCTACGTAGTCATTCTTTCCTGTACCATCTTCTTCACTTG | |
| Arabidopsis thaliana | ATCCTAATGGAAAAGGCAATAAGATTGG | |
| Ser8Term | CCAATCTTATTGCCTTTTCCATTAGGATCAAGTGAAGAAGATGGTA | 6406 |
| TCG-TAG | CAGGAAAGAATGACTACGTAGCAGAGGTGGCCACCATGACGAGG | |
| | AGATGAAGCGTTCAAAGACACTGCCACCAAA | |
| | TGCTACGTAGTCATTCT | 6407 |
| | AGAATGACTACGTAGCA | 6408 |
| Reduced palmitate | GGTGGCAGTGTCTTTGAACGCTTCATCTCCTCGTCATGGTGGCCA | 6409 |
| Acyl-ACP-thioesterase | CCTCTGCTACGTCGTGATTCTTTCCTGTACCATCTTCTTCACTTGAT | |
| Arabidopsis thaliana | CCTAATGGAAAAGGCAATAAGATTGGGTC | |
| Ser9Term | GACCCAATCTTATTGCCTTTTCCATTAGGATCAAGTGAAGAAGATG | 6410 |
| TCA-TGA | GTACAGGAAAGAATCACGACGTAGCAGAGGTGGCCACCATGACG | |
| | AGGAGATGAAGCGTTCAAAGACACTGCCACC | |
| | TACGTCGTGATTCTTTC | 6411 |
| | GAAAGAATCACGACGTA | 6412 |
| Reduced palmitate | ATCTCCTCGTCATGGTGGCCACCTCTGCTACGTCGTCATTCTTTCC | 6413 |
| Acyl-ACP-thioesterase | TGTACCATCTTCTTGACTTGATCCTAATGGAAAAGGCAATAAGATT | |
| Arabidopsis thaliana | GGGTCTACGAATCTTGCTGGACTCAATTC | |
| Ser17Term | GAATTGAGTCCAGCAAGATTCGTAGACCCAATCTTATTGCCTTTTC | 6414 |
| TCA-TGA | CATTAGGATCAAGTCAAGAAGATGGTACAGGAAAGAATGACGACG | |
| | TAGCAGAGGTGGCCACCATGACGAGGAGAT | |
| | ATCTTCTTGACTTGATC | 6415 |
| | GATCAAGTCAAGAAGAT | 6416 |
| Reduced palmitate | GTGGCCACCTCTGCTACGTCGTCATTCTTTCCTGTACCATCTTCTT | 6417 |
| Acyl-ACP-thioesterase | CACTTGATCCTAATTGAAAAGGCAATAAGATTGGGTCTACGAATCT | |
| Arabidopsis thaliana | TGCTGGACTCAATTCTGCACCTAACTCTG | |
| Gly22Term | CAGAGTTAGGTGCAGAATTGAGTCCAGCAAGATTCGTAGACCCAA | 6418 |
| GGA-TGA | TCTTATTGCCTTTTCAATTAGGATCAAGTGAAGAAGATGGTACAGG | |
| | AAAGAATGACGACGTAGCAGAGGTGGCCAC | |
| | ATCCTAATTGAAAAGGC | 6419 |
| | GCCTTTTCAATTAGGAT | 6420 |
| Reduced palmitate | GCTTGAATTTGTGATCTGATTGGTTAATTGTGGCCACAATGGTTGC | 6421 |
| Acyl-ACP-thioesterase | TACTGCCGCCACGTGATCATTCTTTCCGTTGACTTCCCCTTCTGGG | |
| Garcinia mangostana | GATGCCAAATCGGGCAATCCCGGAAAAGG | |
| Ser8Term | CCTTTTCCGGGATTGCCCGATTTGGCATCCCCAGAAGGGGAAGTC | 6422 |
| TCA-TGA | AACGGAAAGAATGATCACGTGGCGGCAGTAGCAACCATTGTGGCC | |
| | ACAATTAACCAATCAGATCACAAATTCAAGC | |
| | CGCCACGTGATCATTCT | 6423 |
| | AGAATGATCACGTGGCG | 6424 |
| Reduced palmitate | TGAATTTGTGATCTGATTGGTTAATTGTGGCCACAATGGTTGCTAC | 6425 |
| Acyl-ACP-thioesterase | TGCCGCCACGTCATGATTCTTTCCGTTGACTTCCCCTTCTGGGGAT | |
| Garcinia mangostana | GCCAAATCGGGCAATCCCGGAAAAGGGTC | |
| Ser9Term | GACCCTTTTCCGGGATTGCCCGATTTGGCATCCCCAGAAGGGGAA | 6426 |
| TCA-TGA | GTCAACGGAAAGAATCATGACGTGGCGGCAGTAGCAACCATTGTG | |
| | GCCACAATTAACCAATCAGATCACAAATTCA | |
| | CACGTCATGATTCTTTC | 6427 |
| | GAAAGAATCATGACGTG | 6428 |
| Reduced palmitate | CTGATTGGTTAATTGTGGCCACAATGGTTGCTACTGCCGCCACGT | 6429 |
| Acyl-ACP-thioesterase | CATCATTCTTTCCGTAGACTTCCCCTTCTGGGGATGCCAAATCGGG | |
| Garcinia mangostana | CAATCCCGGAAAAGGGTCGGTGAGTTTTGG | |
| Leu13Term | CCAAAACTCACCGACCCTTTTCCGGGATTGCCCGATTTGGCATCC | 6430 |
| TTG-TAG | CCAGAAGGGGAAGTCTACGGAAAGAATGATGACGTGGCGGCAGT | |
| | AGCAACCATTGTGGCCACAATTAACCAATCAG | |
| | CTTTCCGTAGACTTCCC | 6431 |
| | GGGAAGTCTACGGAAAG | 6432 |
| Reduced palmitate | ATGGTTGCTACTGCCGCCACGTCATCATTCTTTCCGTTGACTTCCC | 6433 |
| Acyl-ACP-thioesterase | CTTCTGGGGATGCCTAATCGGGCAATCCCGGAAAAGGGTCGGTG | |
| Garcinia mangostana | AGTTTTGGGTCAATGAAGTCGAAATCCGCGG | |
| Lys21Term | CCGCGGATTTCGACTTCATTGACCCAAAACTCACCGACCCTTTTCC | 6434 |
| AAA-TAA | GGGATTGCCCGATTAGGCATCCCCAGAAGGGGAAGTCAACGGAA | |
| | AGAATGATGACGTGGCGGCAGTAGCAACCAT | |
| | GGGATGCCTAATCGGGC | 6435 |
| | GCCCGATTAGGCATCCC | 6436 |
| Reduced palmitate | GGGATTTCAGCACGAAATTGAAGTTGTTTTTAAAAACCATGGTTGC | |
| Acyl-ACP-thioesterase | TACTGCTGTGACATAGGCGTTTTTCCCAGTCACTTCTTCACCTGAC | |
| Gossypium hirsutum | TCCTCTGACTCGAAAAACAAGAAGCTCGG | |
| Ser8Term | CCGAGCTTCTTGTTTTTCGAGTCAGAGGAGTCAGGTGAAGAAGTG | 6438 |
| TCG-TAG | ACTGGGAAAAACGCCTATGTCACAGCAGTAGCAACCATGGTTTTTA | |
| | AAAACAACTTCAATTTCGTGCTGAAATCCC | |
| | TGTGACATAGGCGTTTT | 6439 |
| | AAAACGCCTATGTCACA | 6440 |
| Reduced palmitate | TGTTTTTAAAAACCATGGTTGCTACTGCTGTGACATCGGCGTTTTT | 6441 |
| Acyl-ACP-thioesterase | CCCAGTCACTTCTTGACCTGACTCCTCTGACTCGAAAAACAAGAAG | |
| Gossypium hirsutum | CTCGGAAGCATCAAGTCGAAGCCATCGGT | |
| Ser16Term | ACCGATGGCTTCGACTTGATGCTTCCGAGCTTCTTGTTTTTCGAGT | 6442 |
| TCA-TGA | CAGAGGAGTCAGGTCAAGAAGTGACTGGGATAAAACGCCGATGTCA | |
| | CAGCAGTAGCAACCATGGTTTTTAAAAACA | |
| | CACTTCTTGACCTGACT | 6443 |
| | AGTCAGGTCAAGAAGTG | 6444 |
| Reduced palmitate | TTGCTACTGCTGTGACATCGGCGTTTTTCCCAGTCACTTCTTCACC | 6445 |
| Acyl-ACP-thioesterase | TGACTCCTCTGACTAGAAAAACAAGAAGCTCGGAAGCATCAAGTC | |
| Gossypium hirsutum | GAAGCCATCGGTTTCTTCTGGAAGTTTGCA | |
| Ser22Term | TGCAAACTTCCAGAAGAAACCGATGGCTTCGACTTGATGCTTCCG | 6446 |
| TCG-TAG | AGCTTCTTGTTTTTCTAGTCAGAGGAGTCAGGTGAAGAAGTGACTG | |
| | GGAAAAACGCCGATGTCACAGCAGTAGCAA | |
| | CTCTGACTAGAAAAACA | 6447 |
| | TGTTTTTCTAGTCAGAG | 6448 |
| Reduced palmitate | GCTACTGCTGTGACATCGGCGTTTTTCCCAGTCACTTCTTCACCTG | 6449 |
| Acyl-ACP-thioesterase | ACTCCTCTGACTCGTAAAACAAGAAGCTCGGAAGCATCAAGTCGA | |
| Gossypium hirsutum | AGCCATCGGTTTCTTCTGGAAGTTTGCAAG | |
| Lys23Term | CTTGCAAACTTCCAGAAGAAACCGATGGCTTCGACTTGATGCTTCC | 6450 |
| AAA-TAA | GAGCTTCTTGTTTTACGAGTCAGAGGAGTCAGGTGAAGAAGTGAC | |
| | TGGGAAAAACGCCGATGTCACAGCAGTAGC | |
| | CTGACTCGTAAAACAAG | 6451 |
| | CTTGTTTTACGAGTCAG | 6452 |
| Reduced palmitate | CTCCCGCTCGTTGAAAGACAATGGTGGCTACCGCTGCAAGCTCTG | 6453 |
| Acyl-ACP-thioesterase | CATTCTTCCCCGTGTAGTCCCCGGTCACCTCCTCTAGACCAGGAA | |
| Cuphea hookeriana | AGCCCGGAAATGGGTCATCGAGCTTCAGCCC | |
| Ser14Term | GGGCTGAAGCTCGATGACCCATTTCCGGGCTTTCCTGGTCTAGAG | 6454 |
| TCG-TAG | GAGGTGACCGGGGACTACACGGGGAAGAATGCAGAGCTTGCAGC | |
| | GGTAGCCACCATTGTCTTTCAACGAGCGGGAG | |
| | CCCCGTGTAGTCCCCGG | 6455 |
| | CCGGGGACTACACGGGG | 6456 |
| Reduced palmitate | ATGGTGGCTACCGCTGCAAGCTCTGCATTCTTCCCCGTGTCGTCC | 6457 |
| Acyl-ACP-thioesterase | CCGGTCACCTCCTCTTGACCAGGAAAGCCCGGAAATGGGTCATCG | |
| Cuphea hookeriana | AGCTTCAGCCCCATCAAGCCCAAATTTGTCG | |
| Arg21Term | CGACAAATTTGGGCTTGATGGGGCTGAAGCTCGATGACCCATTTC | 6458 |
| AGA-TGA | CGGGCTTTCCTGGTCAAGAGGAGGTGACCGGGGACGACACGGG | |
| | GAAGAATGCAGAGCTTGCAGCGGTAGCCACCAT | |
| | CCTCCTCTTGACCAGGA | 6459 |
| | TCCTGGTCAAGAGGAGG | 6460 |
| Reduced palmitate | GCTACCGCTGCAAGCTCTGCATTCTTCCCCGTGTCGTCCCCGGTC | 6461 |
| Acyl-ACP-thioesterase | ACCTCCTCTAGACCATGAAAGCCCGGAAATGGGTCATCGAGCTTC | |
| Cuphea hookeriana | AGCCCCATCAAGCCCAAATTTGTCGCCAATG | |
| Gly23Term | CATTGGCGACAAATTTGGGCTTGATGGGGCTGAAGCTCGATGACC | 6462 |
| GGA-TGA | CATTTCCGGGCTTTCATGGTCTAGAGGAGGTGACCGGGGACGAC | |
| | ACGGGGAAGAATGCAGAGCTTGCAGCGGTAGC | |
| | CTAGACCATGAAAGCCC | 6463 |
| | GGGCTTTCATGGTCTAG | 6464 |
| Reduced palmitate | ACCGCTGCAAGCTCTGCATTCTTCCCCGTGTCGTCCCCGGTCACC | 6465 |
| Acyl-ACP-thioesterase | TCCTCTAGACCAGGATAGCCCGGAAATGGGTCATCGAGCTTCAGC | |
| Cuphea hookeriana | CCCATCAAGCCCAAATTTGTCGCCAATGGCG | |
| Lys24Term | CGCCATTGGCGACAAATTTTGGGCTTGATGGGGCTGAAGCTCGATG | 6466 |
| AAG-TAG | ACCCATTTCCGGGCTATCCTGGTCTAGAGGAGGTGACCGGGGAC | |
| | GACACGGGGAAGAATGCAGAGCTTGCAGCGGT | |
| | GACCAGGATAGCCCGGA | 6467 |
| | TCCGGGCTATCCTGGTC | 6468 |
| Reduced palmitate | GCCACCGCTGCAAGTTCTGCATTCTTCCCCCTGCCGTCCCCGGAC | 6469 |
| Acyl-ACP-thioesterase | ACCTCCTCTAGGCCGTGAAAGCTCGGAAATGGGTCATCGAGCTTG | |
| Cuphea lanceolata | AGCCCCCTCAAGCCCAAATTTGTCGCCAATG | |
| Gly23Term | CATTGGCGACAAATTTGGGCTTGAGGGGGCTCAAGCTCGATGACC | 6470 |
| GGA-TGA | CATTTCCGAGCTTTCACGGCCTAGAGGAGGTGTCCGGGGACGGC | |
| | AGGGGGAAGAATGCAGAACTTGCAGCGGTGGC | |
| | CTAGGCCGTGAAAGCTC | 6471 |
| | GAGCTTTCACGGCCTAG | 6472 |
| Reduced palmitate | ACCGCTGCAAGTTCTGCATTCTTCCCCCTGCCGTCCCCGGACACC | 6473 |
| Acyl-ACP-thioesterase | TCCTCTAGGCCGGGATAGCTCGGAAATGGGTCATCGAGCTTGAGC | |
| Cuphea lanceolata | CCCCTCAAGCCCAAATTTGTCGCCAATGCCG | |
| Lys24Term | CGGCATTGGCGACAAATTTGGGCTTGAGGGGGCTCAAGCTCGAT | 6474 |
| AAG-TAG | GACCCATTTCCGAGCTATCCCGGCCTAGAGGAGGTGTCCGGGGA | |
| | CGGCAGGGGGAAGAATGCAGAACTTGCAGCGGT | |
| | GGCCGGGATAGCTCGGA | 6475 |
| | TCCGAGCTATCCCGGCC | 6476 |
| Reduced palmitate | GCAAGTTCTGCATTCTTCCCCCTGCCGTCCCCGGACACCTCCTCT | 6477 |
| Acyl-ACP-thioesterase | AGGCCGGGAAAGCTCTGAAATGGGTCATCGAGCTTGAGCCCCCT | |
| Cuphea lanceolata | CAAGCCCAAATTTGTCGCCAATGCCGGGTTGA | |
| Gly26Term | TCAACCCGGCATTGGCGACAAATTTGGGCTTGAGGGGGCTCAAGC | 6478 |
| GGA-TGA | TCGATGACCCATTTCAGAGCTTTCCCGGCCTAGAGGAGGTGTCCG | |
| | GGGACGGCAGGGGGAAGAATGCAGAACTTGC | |
| | GAAAGCTCTGAAATGGG | 6479 |
| | CCCATTTCAGAGCTTTC | 6480 |
| Reduced palmitate | CATTCTTCCCCCTGCCGTCCCCGGACACCTCCTCTAGGCCGGGAA | 6481 |
| Acyl-ACP-thioesterase | AGCTCGGAAATGGGTGATCGAGCTTGAGCCCCCTCAAGCCCAAAT | |
| Cuphea lanceolata | TTGTCGCCAATGCCGGGTTGAAGGTTAAGGC | |
| Ser29term | GCCTTAACCTTCAACCCGGCATTGGCGACAAATTTGGGCTTGAGG | 6482 |
| TCA-TGA | GGGCTCAAGCTCGATCACCCATTTCCGAGCTTTCCCGGCCTAGAG | |
| | GAGGTGTCCGGGGACGGCAGGGGGAAGAATG | |
| | AAATGGGTGATCGAGCT | 6483 |
| | AGCTCGATCACCCATTT | 6484 |
| Reduced palmitate | CGTTTAAGTGGATCGGACATTTAAGTGTTTTAATCATGGTAGCTAT | 6485 |
| Acyl-ACP-thioesterase | GAGTGCTACTGCGTAGCTGTTTCCGGTTTCTTCCCCAAAACCTCAC | |
| Helianthus annuus | TCTGGAGCCAAGACATCTGATAAGCTTGG | |
| Ser9Term | CCAAGCTTATCAGATGTCTTGGCTCCAGAGTGAGGTTTTGGGGAA | 6486 |
| TCG-TAG | GAAACCGGAAACAGCTACGCAGTAGCACTCATAGCTACCATGATT | |
| | AAAACACTTAAATGTCCGATCCACTTAAACG | |
| | TACTGCGTAGCTGTTTC | 6487 |
| | GAAACAGCTACGCAGTA | 6488 |
| Reduced palmitate | AGTGTTTTAATCATGGTAGCTATGAGTGCTACTGCGTCGCTGTTTC | 6489 |
| Acyl-ACP-thioesterase | CGGTTTCTTCCCCATAACCTCACTCTGGAGCCAAGACATCTGATAA | |
| Helianthus annuus | GCTTGGAGGTGAACCAGGTAGTGTTGCTG | |
| Lys17Term | CAGCAACACTACCTGGTTCACCTCCAAGCTTATCAGATGTCTTGGC | 6490 |
| AAA-TAA | TCCAGAGTGAGGTTATGGGGAAGAAACCGGAAACAGCGACGCAG | |
| | TAGCACTCATAGCTACCATGATTAAAACACT | |
| | CTTCCCCATAACCTCAC | 6491 |
| | GTGAGGTTATGGGGAAG | 6492 |
| Reduced palmitate | ATGGTAGCTATGAGTGCTACTGCGTCGCTGTTTCCGGTTTCTTCCC | 6493 |
| Acyl-ACP-thioesterase | CAAAACCTCACTCTTGAGCCAAGACATCTGATAAGCTTGGAGGTG | |
| Helianthus annuus | AACCAGGTAGTGTTGCTGTGCGCGGAATCA | |
| Gly21Term | TGATTCCGCGCACAGCAACACTACCTGGTTCACCTCCAAGCTTATC | 6494 |
| GGA-TGA | AGATGTCTTGGCTCAAGAGTGAGGTTTTGGGGAAGAAACCGGAAA | |
| | CAGCGACGCAGTAGCACTCATAGCTACCAT | |
| | CTCACTCTTGAGCCAAG | 6495 |
| | CTTGGCTCAAGAGTGAG | 6496 |
| Reduced palmitate | GCTATGAGTGCTACTGCGTCGCTGTTTCCGGTTTCTTCCCCAAAAC | 6497 |
| Acyl-ACP-thioesterase | CTCACTCTGGAGCCTAGACATCTGATAAGCTTGGAGGTGAACCAG | |
| Helianthus annuus | GTAGTGTTGCTGTGCGCGGAATCAAGACAA | |
| Lys23Term | TTGTCTTGATTCCGCGCACAGCAACACTACCTGGTTCACCTCCAAG | 6498 |
| AAG-TAG | CTTATCAGATGTCTAGGCTCCAGAGTGAGGTTTTGGGGAAGAAAC | |
| | CGGAAACAGCGACGCAGTAGCACTCATAGC | |
| | CTGGAGCCTAGACATCT | 6499 |
| | AGATGTCTAGGCTCCAG | 6500 |
| Reduced palmitate | ATGGTGGCTGCTGCAGCAAGTTCTGCATGCTTCCCTGTTCCATCC | 6501 |
| Acyl-ACP-thioesterase | CCAGGAGCCTCCCCTTAACCTGGGAAGTTAGGCAACTGGTCATCG | |
| Cuphea palustris | AGTTTGAGCCCTTCCTTGAAGCCCAAGTCAA | |
| Lys21Term | TTGACTTGGGCTTCAAGGAAGGGCTCAAACTCGATGACCAGTTGC | 6502 |
| AAA-TAA | CTAACTTCCCAGGTTAAGGGGAGGCTCCTGGGGATGGAACAGGG | |
| | AAGCATGCAGAACTTGCTGCAGCAGCCACCAT | |
| | CCTCCCCTTAACCTGGG | 6503 |
| | CCCAGGTTAAGGGGAGG | 6504 |
| Reduced palm itate | GCTGCAGCAAGTTCTGCATGCTTCCCTGTTCCATCCCCAGGAGCC | 6505 |
| Acyl-ACP-thioesterase | TCCCCTAAACCTGGGTAGTTAGGCAACTGGTCATCGAGTTTGAGC | |
| Cuphea palustris | CCTTCCTTGAAGCCCAAGTCAATCCCCAATG | |
| Lys24Term | CATTGGGGATTGACTTGGGCTTCAAGGAAGGGCTCAAACTCGATG | 6506 |
| AAG-TAG | ACCAGTTGCCTAACTACGCAGGTTTAGGGGAGGCTCCTGGGGATG | |
| | GAACAGGGAAGCATGCAGAACTTGCTGCAGC | |
| | AACCTGGGTAGTTAGGC | 6507 |
| | GCCTAACTACCCAGGTT | 6508 |
| Reduced palmitate | TGCATGCTTCCCTGTTCCATCCCCAGGAGCCTCCCCTAAACCTGG | 6509 |
| Acyl-ACP-thioesterase | GAAGTTAGGCAACTGATCATCGAGTTTGAGCCCTTCCTTGAAGCC | |
| Cuphea palustris | CAAGTCAATCCCCAATGGCGGATTTCAGGTT | |
| Trp28Term | AACCTGAAATCCGCCATTGGGGATTGACTTGGGCTTCAAGGAAGG | 6510 |
| TGG-TGA | GCTCAAACTCGATGATCAGTTGCCTAACTTCCCAGGTTTAGGGGA | |
| | GGCTCCTGGGGATGGAACAGGGAAGCATGCA | |
| | GGCAACTGATCATCGAG | 6511 |
| | CTCGATGATCAGTTGCC | 6512 |
| Reduced palmitate | CATGCTTCCCTGTTCCATCCCCAGGAGCCTCCCCTAAACCTGGGA | 6513 |
| Acyl-ACP-thioesterase | AGTTAGGCAACTGGTGATCGAGTTTGAGCCCTTCCTTGAAGCCCA | |
| Cuphea palustris | AGTCAATCCCCAATGGCGGATTTCAGGTTAA | |
| Ser29Term | TTAACCTGAAATCCGCCATTGGGGATTGACTTGGGCTTCAAGGAA | 6514 |
| TCA-TGA | GGGCTCAAACTCGATCACCAGTTGCCTAACTTCCCAGGTTTAGGG | |
| | GAGGCTCCTGGGGATGGAACAGGGAAGCATG | |
| | CAACTGGTGATCGAGTT | 6515 |
| | AACTCGATCACCAGTTG | 6516 |
| Reduced palmitate | ATGGTGGCTGCCGCAGCAAGTTCTGCATTCTTCTCCGTTCCAACC | 6517 |
| Acyl-ACP-thioesterase | CCGGGAATCTCCCCTTAACCCGGGAAGTTCGGTAATGGTGGCTTT | |
| Cuphea hookeriana | CAGGTTAAGGCAAACGCCAATGCCCATCCTA | |
| Lys21Term | TAGGATGGGCATTGGCGTTTGCCTTAACCTGAAAGCCACCATTAC | 6518 |
| AAA-TAA | CGAACTTCCCGGGTTAAGGGGAGATTCCCGGGGTTGGAACGGAG | |
| | AAGAATGCAGAACTTGCTGCGGCAGCCACCAT | |
| | TCTCCCCTTAACCCGGG | 6519 |
| | CCCGGGTTAAGGGGAGA | 6520 |
| Reduced palmitate | GCCGCAGCAAGTTCTGCATTCTTCTCCGTTCCAACCCCGGGAATC | 6521 |
| Acyl-ACP-thioesterase | TCCCCTAAACCCGGGTAGTTCGGTAATGGTGGCTTTCAGGTTAAG | |
| Cuphea hookeriana | GCAAACGCCAATGCCCATCCTAGTCTAAAGT | |
| Lys24Term | ACTTTAGACTAGGATGGGCATTGGCGTTTGCCTTAACCTGAAAGC | 6522 |
| AAG-TAG | CACCATTACCGAACTACCCGGGTTTAGGGGAGATTCCCGGGGTTG | |
| | GAACGGAGAAGAATGCAGAACTTGCTGCGGC | |
| | AACCCGGGTAGTTCGGT | 6523 |
| | ACCGAACTACCCGGGTT | 6524 |
| Reduced palmitate | TTCTCCGTTCCAACCCCGGGAATCTCCCCTAAACCCGGGAAGTTC | 6525 |
| Acyl-ACP-thioesterase | GGTAATGGTGGCTTTTAGGTTAAGGCAAACGCCAATGCCCATCCT | |
| Cuphea hookeriana | AGTCTAAAGTCTGGCAGCCTCGAGACTGAAG | |
| Gln31Term | CTTCAGTCTCGAGGCTGCCAGACTTTAGACTAGGATGGGCATTGG | 6526 |
| CAG-TAG | CGTTTGCCTTAACCTAAAAGCCACCATTACCGAACTTCCCGGGTTT | |
| | AGGGGAGATTCCCGGGGTTGGAACGGAGAA | |
| | GTGGCTTTTAGGTTAAG | 6527 |
| | CTTAACCTAAAAGCCAC | 6528 |
| Reduced palmitate | GTTCCAACCCCGGGAATCTCCCCTAAACCCGGGAAGTTCGGTAAT | 6529 |
| Acyl-ACP-thioesterase | GGTGGCTTTCAGGTTTAGGCAAACGCCAATGCCCATCCTAGTCTA | |
| Cuphea hookeriana | AAGTCTGGCAGCCTCGAGACTGAAGATGACA | |
| Lys33Term | TGTCATCTTCAGTCTCGAGGCTGCCAGACTTTAGACTAGGATGGG | 6530 |
| AAG-TAG | CATTGGCGTTTGCCTAAACCTGAAAGCCACCATTACCGAACTTCCC | |
| | GGGTTTAGGGGAGATTCCCGGGGTTGGAAC | |
| | TTCAGGTTTAGGCAAAC | 6531 |
| | GTTTGCCTAAACCTGAA | 6532 |
| Reduced palmitate | ATGTTGAAGCTCTCGTGTAATGCGACTGATAAGTTACAGACCCTCT | 6533 |
| Acyl-ACP-thioesterase | TCTCGCATTCTCATTAACCGGATCCGGCACACCGGAGAACCGTCT | |
| Brassica rapa | CCTCCGTGTCGTGCTCTCATCTGAGGAAAC | |
| Gln21Term | GTTTCCTCAGATGAGAGCACGACACGGAGGAGACGGTTCTCCGGT | 6534 |
| CAA-TAA | GTGCCGGATCCGGTTAATGAGAATGCGAGAAGAGGGTCTGTAACT | |
| | TATCAGTCGCATTACACGAGAGCTTCAACAT | |
| | ATTCTCATTAACCGGAT | 6535 |
| | ATCCGGTTAATGAGAAT | 6536 |
| Reduced palmitate | GCGACTGATAAGTTACAGACCCTCTTCTCGCATTCTCATCAACCGG | 6537 |
| Acyl-ACP-thioesterase | ATCCGGCACACCGGTGAACCGTCTCCTCCGTGTCGTGCTCTCATC | |
| Brassica rapa | TGAGGAAACCGGTTCTCGATCCTTTGCGAG | |
| Arg28Term | CTCGCAAAGGATCGAGAACCGGTTTCCTCAGATGAGAGCACGACA | 6538 |
| AGA-TGA | CGGAGGAGACGGTTCACCGGTGTGCCGGATCCGGTTGATGAGAA | |
| | TGCGAGAAGAGGGTCTGTAACTTATCAGTCGC | |
| | CACACCGGTGAACCGTC | 6539 |
| | GACGGTTCACCGGTGTG | 6540 |
| Reduced palmitate | CCCTCTTCTCGCATTCTCATCAACCGGATCCGGCACACCGGAGAA | 6541 |
| Acyl-ACP-thioesterase | CCGTCTCCTCCGTGTAGTGCTCTCATCTGAGGAAACCGGTTCTCG | |
| Brassica rapa | ATCCTTTGCGAGCGATCGTATCTGCTGATCA | |
| Ser24Term | TGATCAGCAGATACGATCGCTCGCAAAGGATCGAGAACCGGTTTC | 6542 |
| TCG-TAG | CTCAGATGAGAGCACTACACGGAGGAGACGGTTCTCCGGTGTGC | |
| | CGGATCCGGTTGATGAGAATGCGAGAAGAGGG | |
| | CTCCGTGTAGTGCTCTC | 6543 |
| | GAGAGCACTACACGGAG | 6544 |
| Reduced palmitate | CTTCTCGCATTCTCATCAACCGGATCCGGCACACCGGAGAACCGT | 6545 |
| Acyl-ACP-thioesterase | CTCCTCCGTGTCGTGATCTCATCTGAGGAAACCGGTTCTCGATCC | |
| Brassica rapa | TTTGCGAGCGATCGTATCTGCTGATCAAGGA | |
| Cys25Term | TCCTTGATCAGCAGATACGATCGCTCGCAAAGGATCGAGAACCGG | 6546 |
| TGC-TGA | TTTCCTCAGATGAGATCACGACACGGAGGAGACGGTTCTCCGGTG | |
| | TGCCGGATCCGGTTGATGAGAATGCGAGAAG | |
| | GTGTCGTGATCTCATCT | 6547 |
| | AGATGAGATCACGACAC | 6548 |
| Reduced palmitate | ATTCTTCTTCTATAAACCAAAACCTCAGGAACCATAAAAAAAAAAGG | 6549 |
| Acyl-ACP-thioesterase | GCATCAAAAATGTAGAAGCTTTCGTGTAATGTGACTAACAACTTAC | |
| Brassica napus | ACACCTTCTCCTTCTTCTCCGATTCCTC | |
| Leu2Term | GAGGAATCGGAGAAGAAGGAGAAGGTGTGTAAGTTGTTAGTCACA | 6550 |
| TTG-TAG | TTACACGAAAGCTTCTACATTTTTGATGCCCTTTTTTTTTTATGGTTC | |
| | CTGAGGTTTTGGTTTATAGAAGAAGAAT | |
| | AAAAATGTAGAAGCTTT | 6551 |
| | AAAGCTTCTACATTTTT | 6552 |
| Reduced palmitate | TCTTCTTCTATAAACCAAAACCTCAGGAACCATAAAAAAAAAAGGG | 6553 |
| Acyl-ACP-thioesterase | CATCAAAAATGTTGTAGCTTTCGTGTAATGTGACTAACAACTTACAC | |
| Brassica napus | ACCTTCTCCTTCTTCTCCGATTCCTCCC | |
| Lys3Term | GGGAGGAATCGGAGAAGAAGGAGAAGGTGTGTAAGTTGTTAGTCA | 6554 |
| AAG-TAG | CATTACACGAAAGCTACAACATTTTTGATGCCCTTTTTTTTTTATGG | |
| | TTCCTGAGGTTTTGGTTTATAGAAGAAGA | |
| | AAATGTTGTAGCTTTCG | 6555 |
| | CGAAAGCTACAACATTT | 6556 |
| Reduced palmitate | CTATAAACCAAAACCTCAGGAACCATAAAAAAAAAAGGGCATCAAA | 6557 |
| Acyl-ACP-thioesterase | AATGTTGAAGCTTTAGTGTAATGTGACTAACAACTTACACACCTTCT | |
| Brassica napus | CCTTCTTCTCCGATTCCTCCCTTTTCAT | |
| Ser5Term | ATGAAAAGGGAGGAATCGGAGAAGAAGGAGAAGGTGTGTAAGTT | 6558 |
| TCG-TAG | GTTAGTCACATTACACTAAAGCTTCAACATTTTTGATGCCCTTTTTT | |
| | TTTTATGGTTCCTGAGGTTTTGGTTTATAG | |
| | GAAGCTTTAGTGTAATG | 6559 |
| | CATTACACTAAAGCTTC | 6560 |
| Reduced palmitate | AAACCAAAACCTCAGGAACCATAAAAAAAAAAGGGCATCAAAAATG | 6561 |
| Acyl-ACP-thioesterase | TTGAAGCTTTCGTGAAATGTGACTAACAACTTACACACCTTCTCCTT | |
| Brassica napus | CTTCTCCGATTCCTCCCTTTTCATCCCG | |
| Cys6Term | CGGGATGAAAAGGGAGGAATCGGAGAAGAAGGAGAAGGTGTGTA | 6562 |
| TGT-TGA | AGTTGTTAGTCACATTTCACGAAAGCTTCAACATTTTTGATGCCCTT | |
| | TTTTTTTTATGGTTCCTGAGGTTTTGGTTT | |
| | CTTTCGTGAAATGTGAC | 6563 |
| | GTCACATTTCACGAAAG | 6564 |

TABLE 41
Oligonucleotides to produce plants with increased stearate

| Phenotype, Gene, | | |
|---|---|---|
| Plant & Targeted | | SEQ ID |
| Alteration | Altering Oligos | NO: |
| Increased stearate | GGGAGAGCTCTAGCTCTGTAGAAAAGAAGGATTCATTCATCATATC | 6565 |
| stearoyl-ACP | CAGAAATGGCTCTATAGTTTAACCCTTTGGTGGCATCTCAGCCTTA | |
| desaturase | CAAATTCCCTTCCTCGACTCGTCCGCCAA | |
| Arabidopsis thaliana | TTGGCGGACGAGTCGAGGAAGGGAATTTGTAAGGCTGAGATGCC | 6566 |
| Lys4Term | ACCAAAGGGTTAAACTATAGAGCCATTTCTGGATATGATGAATGAA | |
| AAG-TAG | TCCTTCTTTTCTACAGAGCTAGAGCTCTCCC | |
| | TGGCTCTATAGTTTAAC | 6567 |
| | GTTAAACTATAGAGCCA | 6568 |
| Increased stearate | CTCTGTAGAAAAGAAGGATTCATTCATCATATCCAGAAATGGCTCT | 6569 |
| stearoyl-ACP | AAAGTTTAACCCTTAGGTGGCATCTCAGCCTTACAAATTCCCTTCC | |
| desaturase | TCGACTCGTCCGCCAACTCCTTCTTTCAG | |
| Arabidopsis thaliana | CTGAAAGAAGGAGTTGGCGGACGAGTCGAGGAAGGGAATTTGTA | 6570 |
| Leu8Term | AGGCTGAGATGCCACCTAAGGGTTAAACTTTAGAGCCATTTCTGG | |
| TTG-TAG | ATATGATGAATGAATCCTTCTTTTCTACAGAG | |
| | TAACCCTTAGGTGGCAT | 6571 |
| | ATGCCACCTAAGGGTTA | 6572 |
| Increased stearate | AGAAGGATTCATTCATCATATCCAGAAATGGCTCTAAAGTTTAACC | 6573 |
| stearoyl-ACP | CTTTGGTGGCATCTTAGCCTTACAAATTCCCTTCCTCGACTCGTCC | |
| desaturase | GCCAACTCCTTCTTTCAGATCTCCCAAGT | |
| Arabidopsis thaliana | ACTTGGGAGATCTGAAAGAAGGAGTTGGCGGACGAGTCGAGGAA | 6574 |
| Gln12Term | GGGAATTTGTAAGGCTAAGATGCCACCAAAGGGTTAAACTTTAGA | |
| CAG-TAG | GCCATTTCTGGATATGATGAATGAATCCTTCT | |
| | TGGCATCTTAGCCTTAC | 6575 |
| | GTAAGGCTAAGATGCCA | 6576 |
| Increased stearate | TCATTCATCATATCCAGAAATGGCTCTAAAGTTTAACCCTTTGGTG | 6577 |
| stearoyl-ACP | GCATCTCAGCCTTAGAAATTCCCTTCCTCGACTCGTCCGCCAACTC | |
| desaturase | CTTCTTTCAGATCTCCCAAGTTCCTCTGC | |
| Arabidopsis thaliana | GCAGAGGAACTTGGGAGATCTGAAAGAAGGAGTTGGCGGACGAG | 6578 |
| Phe14Term | TCGAGGAAGGGAATTTCTAAGGCTGAGATGCCACCAAAGGGTTAA | |
| TAC-TAG | ACTTTAGAGCCATTTCTGGATATGATGAATGA | |
| | CAGCCTTAGAAATTCCC | 6579 |
| | GGGAATTTCTAAGGCTG | 6580 |
| Increased stearate | GAGAGCTCGCTCGTGTCTGAAAGAACATCAAACCTCGTATCAAAAA | 6581 |
| stearoyl-ACP | AAAGAAAATGGCATAGAAGCTTAACCCTTTGGCATCTCAGCCTTAC | |
| desaturase | AAACTCCCTTCCTCGGCTCGTCCGCCAAT | |
| Brassica napus | ATTGGCGGACGAGCCGAGGAAGGGAGTTTGTAAGGCTGAGATGC | 6582 |
| Leu3Term | CAAAGGGTTAAGCTTCTATGCCATUTCTTTTTTTTGATACGAGGTT | |
| TTG-TAG | TGATGTTCTTTCAGACACGAGCGAGCTCTC | |
| | AATGGCATAGAAGCTTA | 6583 |
| | TAAGCTTCTATGCCATT | 6584 |
| Increased stearate | GAGCTCGCTCGTGTCTGAAAGAACATCTAAACCTCGTATCAAAAAAA | 6585 |
| stearoyl-ACP | AGAAAATGGCATTGTAGCTTAACCCTTTGGCATCTCAGCCTTACAA | |
| desaturase | ACTCCCTTCCTCGGCTCGTCCGCCAATCT | |
| Brassica napus | AGATTGGCGGACGAGCCGAGGAAGGGAGTTTGTAAGGCTGAGAT | 6586 |
| Lys4Term | GCCAAAGGGTTAAGCTACAATGCCATTTTCTTTTTTTTGATACGAG | |
| AAG-TAG | GTTTGATGTTCTTTCAGACACGAGCGAGCTC | |
| | TGGCATTGTAGCTTAAC | 6587 |
| | GTTAAGCTACAATGCCA | 6588 |
| Increased stearate | TCTGAAAGAACATCAAACCTCGTATCAAAAAAAAGAAAATGGCATT | 6589 |
| stearoyl-ACP | GAAGCTTAACCCTTAGGCATCTCAGCCTTACAAACTCCCTTCCTCG | |
| desaturase | GCTCGTCCGCCAATCTCTACTCTCAGATC | |
| Brassica napus | GATCTGAGAGTAGAGATTGGCGGACGAGCCGAGGAAGGGAGTTT | 6590 |
| Leu8Term | GTAAGGCTGAGATGCCTAAGGGTTAAGCTTCAATGCCATTTTCTTT | |
| TTG-TAG | TTTTTGATACGAGGTTTGATGTTCTTTCAGA | |
| | TAACCCTTAGGCATCTC | 6591 |
| | GAGATGCCTAAGGGTTA | 6592 |
| Increased stearate | AACATCAAACCTCGTATCAAAAAAAAGAAAATGGCATTGAAGCTTA | 6593 |
| stearoyl-ACP | ACCCTTTGGCATCTTAGCCTTACAAACTCCCTTCCTCGGCTCGTCC | |
| desaturase | GCCAATCTCTACTCTCAGATCTCCCAAGT | |
| Brassica napus | ACTTGGGAGATCTGAGAGTAGAGATTGGCGGACGAGCCGAGGAA | 6594 |
| Gln11Term | GGGAGTTTGTAAGGCTAAGATGCCAAAGGGTTAAGCTTCAATGCC | |
| CAG-TAG | ATTTTCTTTTTTTTGATACGAGGTTTGATGTT | |
| | TGGCATCTTAGCCTTAC | 6595 |
| | GTAAGGCTAAGATGCCA | 6596 |
| Increased stearate | AACCAAAAGAAAAAGGTAAGAAAAAAAACAATGGCTCTCAAGCTCA | 6597 |
| stearoyl-ACP | ATCCTTTCCTTTCTTAAACCCAAAAGTTACCTTCTTTCGCTCTTCCA | |
| desaturase | CCAATGGCCAGTACCAGATCTCCTAAGT | |
| Ricinus communis | ACTTAGGAGATCTGGTACTGGCCATTGGTGGAAGAGCGAAAGAAG | 6598 |
| Gln27Term | GTAACTTTTGGGTTTAAGAAAGGAAAGGATTGAGCTTGAGAGCCAT | |
| CAA-TAA | TGTTTTTTTTCTTACCTTTTTCTTTTGGTT | |
| | TCCTTTCTTAAACCCAA | 6599 |
| | TTGGGTTTAAGAAAGGA | 6600 |
| Increased stearate | AAGAAAAAGGTAAGAAAAAAAACAATGGCTCTCAAGCTCAATCCTT | 6601 |
| stearoyl-ACP | TCCTTTCTCAAACCTAAAAGTTACCTTCTTTCGCTCTTCCACCAATG | |
| desaturase | GCCAGTACCAGATCTCCTAAGTTCTACA | |
| Ricinus communis | GTAGAACTTAGGAGATCTGGTACTGGCCATTGGTGGAAGAGCGA | 6602 |
| Gln29Term | AAGAAGGTAACTTTTAGGTTTGAGAAAGGAAAGGATTGAGCTTGA | |
| CAA-TAA | GAGCCATTGTTTTTTTTCTTACCTTTTTCTT | |
| | CTCAAACCTAAAAGTTA | 6603 |
| | TAACTTTTAGGTTTGAG | 6604 |
| Increased stearate | AAAAAGGTAAGAAAAAAAACAATGGCTCTCAAGCTCAATCCTTTCC | 6605 |
| stearoyl-ACP | TTTCTCAAACCCAATAGTTACCTTCTTTCGCTCTTCCACCAATGGCC | |
| desaturase | AGTACCAGATCTCCTAAGTTCTACATGG | |
| Ricinus communis | CCATGTAGAACTTAGGAGATCTGGTACTGGCCATTGGTGGAAGAG | 6606 |
| Lys30Term | CGAAAGAAGGTAACTATTGGGTTTGAGAAAGGAAAGGATTGAGCT | |
| AAG-TAG | TGAGAGCCATTGTTTTTTTTCTTACCTTTTT | |
| | AAACCCAATAGTTACCT | 6607 |
| | AGGTAACTATTGGGTTT | 6608 |
| Increased stearate | TCTCAAACCCAAAAGTTACCTTCTTTCGCTCTTCCACCAATGGCCA | 6609 |
| stearoyl-ACP | GTACCAGATCTCCTTAGTTCTACATGGCCTCTACCCTCAAGTCTGG | |
| desaturase | TTCTAAGGAAGTTGAGAATCTCAAGAAGC | |
| Ricinus communis | GCTTCTTGAGATTCTCAACTTCCTTAGAACCAGACTTGAGGGTAGA | 6610 |
| Lys46Term | GGCCATGTAGAACTAAGGAGATCTGGTACTGGCCATTGGTGGAAG | |
| AAG-TAG | AGCGAAAGAAGGTAACTTTTGGGTTTGAGA | |
| | GATCTCCTTAGTTCTAC | 6611 |
| | GTAGAACTAAGGAGATC | 6612 |
| Increased stearate | TCTTCTGATTCATTTAATCTTTACTCATCAATGGCTCTGAGACTGAA | 6613 |
| stearoyl-ACP | CCCTATCCCCACCTAAACCTTCTCCCTCCCCCAAATGGCCAGTCTC | |
| desaturase | AGATCTCCCAGGTTCCGCATGGCCTCTA | |
| Glycine max | TAGAGGCCATGCGGAACCTGGGAGATCTGAGACTGGCCATTTGG | 6614 |
| Gln11Term | GGGAGGGAGAAGGTTTAGGTGGGGATAGGGTTCAGTCTCAGAGC | |
| CAA-TAA | CATTGATGAGTAAAGATTAAATGAATCAGAAGA | |
| | TCCCCACCTAAACCTTC | 6615 |
| | GAAGGTTTAGGTGGGGA | 6616 |
| Increased stearate | CTTTACTCATCAATGGCTCTGAGACTGAACCCTATCCCCACCCAAA | 6617 |
| stearoyl-ACP | CCTTCTCCCTCCCCTAAATGGCCAGTCTCAGATCTCCCAGGTTCC | |
| desaturase | GCATGGCCTCTACCCTCCGCTCCGGTTCCA | |
| Glycine max | TGGAACCGGAGCGGAGGGTAGAGGCCATGCGGAACCTGGGAGAT | 6618 |
| Gln11Term | CTGAGACTGGCCATTTAGGGGAGGGAGAAGGTTTGGGTGGGGAT | |
| CAA-TAA | AGGGTTCAGTCTCAGAGCCATTGATGAGTAAAG | |
| | CCCTCCCCTAAATGGCC | 6619 |
| | GGCCATTTAGGGGAGGG | 6620 |
| Increased stearate | GCTCTGAGACTGAACCCTATCCCCACCCAAACCTTCTCCCTCCCC | 6621 |
| stearoyl-ACP | CAAATGGCCAGTCTCTGATCTCCCAGGTTCCGCATGGCCTCTACC | |
| desaturase | CTCCGCTCCGGTTCCAAAGAGGTTGAAAATA | |
| Glycine max | TATTTTCAACCTCTTTGGAACCGGAGCGGAGGGTAGAGGCCATGC | 6622 |
| Arg22Term | GGAACCTGGGAGATCAGAGACTGGCCATTTGGGGGAGGGAGAAG | |
| AGA-TGA | GTTTGGGTGGGGATAGGGTTCAGTCTCAGAGC | |
| | CCAGTCTCTGATCTCCC | 6623 |
| | GGGAGATCAGAGACTGG | 6624 |
| Increased stearate | CAAATGGCCAGTCTCAGATCTCCCAGGTTCCGCATGGCCTCTACC | 6625 |
| stearoyl-ACP | CTCCGCTCCGGTTCCTAAGAGGTTGAAAATATTAAGAAGCCATTCA | |
| desaturase | CTCCTCCCAGAGAAGTGCATGTTCAAGTAA | |
| Glycine max | TTACTTGAACATGCACTTCTCTGGGAGGAGTGAATGGCTTCTTAAT | 6626 |
| Lys37Term | ATTTTCAACCTCTTAGGAACCGGAGCGGAGGGTAGAGGCCATGCG | |
| AAA-TAA | GAACCTGGGAGATCTGAGACTGGCCATTTG | |
| | CCGGTTCCTAAGAGGTT | 6627 |
| | AACCTCTTAGGAACCGG | 6628 |
| Increased stearate | CAACAAGCACACACAAGAACAACATCAACAATGGCGATTCGCATC | 6629 |
| stearoyl-ACP | AATACGGCGACGTTTTAATCAGACCTGTACCGTTCATTCGCGTTTC | |
| desaturase | CTCAACCGAAACCTCTCAGATCTCCCAAAT | |
| Helianthus annuus | ATTTGGGAGATCTGAGAGGTTTCGGTTGAGGAAACGCGAATGAAC | 6630 |
| Gln11Term | GGTACAGGTCTGATTAAAACGTCGCCGTATTGATGCGAATCGCCA | |
| CAA-TAA | TTGTTGATGTTGTTCTTGTGTGTGCTTGTTG | |
| | CGACGTTTTAATCAGAC | 6631 |
| | GTCTGATTAAAACGTCG | 6632 |
| Increased stearate | AAGCACACACAAGAACAACATCAACAATGGCGATTCGCATCAATAC | 6633 |
| stearoyl-ACP | GGCGACGTTTCAATGAGACCTGTACCGTTCATTCGCGTTTCCTCAA | |
| desaturase | CCGAAACCTCTCAGATCTCCCAAATTCGC | |
| Helianthus annuus | GCGAATTTGGGAGATCTGAGAGGTTTCGGTTGAGGAAACGCGAAT | 6634 |
| Ser12Term | GAACGGTACAGGTCTCATTGAAACGTCGCCGTATTGATGCGAATC | |
| TCA-TGA | GCCATTGTTGATGTTGTTCTTGTGTGTGCTT | |
| | GTTTCAATGAGACCTGT | 6635 |
| | ACAGGTCTCATTGAAAC | 6636 |
| Increased stearate | AAGAACAACATCAACAATGGCGATTCGCATCAATACGGCGACGTTT | 6637 |
| stearoyl-ACP | CAATCAGACCTGTAGCGTTCATTCGCGTTTCCTCAACCGAAACCTC | |
| desaturase | TCAGATCTCCCAAATTCGCCATGGCTTCC | |
| Helianthus annuus | GGAAGCCATGGCGAATTTGGGAGATCTGAGAGGTTTCGGTTGAGG | 6638 |
| Tyr15Term | AAACGCGAATGAACGCTACAGGTCTGATTGAAACGTCGCCGTATT | |
| TAC-TAG | GATGCGAATCGCCATTGTTGATGTTGTTCTT | |
| | GACCTGTAGCGTTCATT | 6639 |
| | AATGAACGCTACAGGTC | 6640 |
| Increased stearate | CAACATCAACAATGGCGATTCGCATCAATACGGCGACGTTTCAATC | 6641 |
| stearoyl-ACP | AGACCTGTACCGTTGATTCGCGTTTCGTCAACCGAAACCTCTCAGA | |
| desaturase | TCTCCCAAATTCGCCATGGCTTCCACCAT | |
| Helianthus annuus | ATGGTGGAAGCCATGGCGAATTTGGGAGATCTGAGAGGTTTCGGT | 6642 |
| Ser17Term | TGAGGAAACGCGAATCAACGGTACAGGTCTGATTGAAACGTCGCC | |
| TCA-TGA | GTATTGATGCGAATCGCCATTGTTGATGTTG | |
| | GTACCGTTGATTCGCGT | 6643 |
| | ACGCGAATCAACGGTAC | 6644 |
| Increased stearate | ACACACAACACACACTCAATCACACACACATCATCATCTTCTTCATC | 6645 |
| stearoyl-ACP | AACGATGGCGCTTTGAATGAGTCCGGTGACGCTTCAACGGGAGAT | |
| desaturase | ATATCCTTCATACACTTTTCATCAATCGA | |
| Helianthus annuus | TCGATTGATGAAAAGTGTATGAAGGATATATCTCCCGTTGAAGCGT | 6646 |
| Arg4Term | CACCGGACTCATTCAAAGCGCCATCGTTGATGAAGAAGATGATGA | |
| CGA-TGA | TGTGTGTGTGATTGAGTGTGTGTTGTGTGT | |
| | TGGCGCTTTGAATGAGT | 6647 |
| | ACTCATTCAAAGCGCCA | 6648 |
| Increased stearate | ACACACACATCATCATCTTCTTCATCAACGATGGCGCTTCGAATGA | 6649 |
| stearoyl-ACP | GTCCGGTGACGCTTTAACGGGAGATATATCCTTCATACACTTTTCA | |
| desaturase | TCAATCGAAAAATCTCAGATCTCCTAAAT | |
| Helianthus annuus | ATTTAGGAGATCTGAGATTTTTCGATTGATGAAAAGTGTATGAAGG | 6650 |
| Gln11Term | ATATATCTCCCGTTAAAGCGTCACCGGACTCATTCGAAGCGCCATC | |
| CAA-TAA | GTTGATGAAGAAGATGATGATGTGTGTGT | |
| | TGACGCTTTAACGGGAG | 6651 |
| | CTCCCGTTAAAGCGTCA | 6652 |
| Increased stearate | ACATCATCATCTTCTTCATCAACGATGGCGCTTCGAATGAGTCCGG | 6653 |
| stearoyl-ACP | TGACGCTTCAACGGTAGATATATCCTTCATACACTTTTCATCAATCG | |
| desaturase | AAAAATCTCAGATCTCCTAAATTCGCGA | |
| Helianthus annuus | TCGCGAATTTAGGAGATCTGAGATTTTTCGATTGATGAAAAGTGTA | 6654 |
| Glu13Term | TGAAGGATATATCTACCGTTGAAGCGTCACCGGACTCATTCGAAG | |
| GAG-TAG | CGCCATCGTTGATGAAGAAGATGATGATGT | |
| | TTCAACGGTAGATATAT | 6655 |
| | ATATATCTACCGTTGAA | 6656 |
| Increased stearate | ATCTTCTTCATCAACGATGGCGCTTCGAATGAGTCCGGTGACGCTT | 6657 |
| stearoyl-ACP | CAACGGGAGATATAGCCTTCATACACTTTTCATCAATCGAAAAATC | |
| desaturase | TCAGATCTCCTAAATTCGCGATGGCTTCC | |
| Helianthus annuus | GGAAGCCATCGCGAATTTAGGAGATCTGAGATTTTTCGATTGATGA | 6658 |
| Tyr15Term | AAAGTGTATGAAGGCTATATCTCCCGTTGAAGCGTCACCGGACTC | |
| TAT-TAG | ATTCGAAGCGCCATCGTTGATGAAGAAGAT | |
| | GAGATATAGCCTTCATA | 6659 |
| | TATGAAGGCTATATCTC | 6660 |
| Increased stearate | AACTCAGCCAGCTTGCCCCCAAACAACAGCGCAGAAAAACCTTCA | 6661 |
| stearoyl-ACP | ACAACAATGGCTCTCTAGCTCAACCCAGTCACCACCTTCCCTTCAA | |
| desaturase | CACGCTCCCTCAACAACTTCTCCTCCAGAT | |
| Linum usitatissimum | ATCTGGAGGAGAAGTTGTTGAGGGAGCGTGTTGAAGGGAAGGTG | 6662 |
| Lys4Term | GTGACTGGGTTGAGCTAGAGAGCCATTGTTGTTGAAGGTTTTTCT | |
| AAG-TAG | GCGCTGTTGTTTGGGGGCAAGCTGGCTGAGTT | |
| | TGGCTCTCTAGCTCAAC | 6663 |
| | GTTGAGCTAGAGAGCCA | 6664 |
| Increased stearate | GCGCAGAAAAACCTTCAACAACAATGGCTCTCAAGCTCAACCCAG | 6665 |
| stearoyl-ACP | TCACCACCTTCCCTTGAACACGCTCCCTCAACAACTTCTCCTCCAG | |
| desaturase | ATCTCCTCGCACCTTTCTCATGGCTGCTTC | |
| Linum usitatissimum | GAAGCAGCCATGAGAAAGGTGCGAGGAGATCTGGAGGAGAAGTT | 6666 |
| Ser13Term | GTTGAGGGAGCGTGTTCAAGGGAAGGTGGTGACTGGGTTGAGCT | |
| TCA-TGA | TGAGAGCCATTGTTGTTGAAGGTTTTTCTGCGC | |
| | CTTCCCTTGAACACGCT | 6667 |
| | AGCGTGTTCAAGGGAAG | 6668 |
| Increased stearate | CTCAAGCTCAACCCAGTCACCACCTTCCCTTCAACACGCTCCCTCA | 6669 |
| stearoyl-ACP | ACAACTTCTCCTCCTGATCTCCTCGCACCTTTCTCATGGCTGCTTC | |
| desaturase | CACTTTCAATTCCACCTCCACCAAGTAAG | |
| Linum usitatissimum | CTTACTTGGTGGAGGTGGAATTGAAAGTGGAAGCAGCCATGAGAA | 6670 |
| Arg23Term | AGGTGCGAGGAGATCAGGAGGAGAAGTTGTTGAGGGAGCGTGTT | |
| AGA-TGA | GAAGGGAAGGTGGTGACTGGGTTGAGCTTGAG | |
| | TCTCCTCCTGATCTCCT | 6671 |
| | AGGAGATCAGGAGGAGA | 6672 |
| Increased stearate | TCCTCCAGATCTCCTCGCACCTTTCTCATGGCTGCTTCCACTTTCA | 6673 |
| stearoyl-ACP | ATTCCACCTCCACCTAGTAAGCATCTCCTCCTCCTCGGAATCTCCG | |
| desaturase | CCGATTTCTTTTAAGCGATTGATCGTAGA | |
| Linum usitatissimum | TCTACGATCAATCGCTTAAAAGAAATCGGCGGAGATTCCGAGGAG | 6674 |
| Lys41Term | GAGGAGATGCTTACTAGGTGGAGGTGGAATTGAAAGTGGAAGCA | |
| AAG-TAG | GCCATGAGAAAGGTGCGAGGAGATCTGGAGGA | |
| | CCTCCACCTAGTAAGCA | 6675 |
| | TGCTTACTAGGTGGAGG | 6676 |
| Increased stearate | ATGGCACTGAAACTTTGCTTTCCACCCCACAAGATGCCTTCCTTCC | 6677 |
| stearoyl-ACP | CCGATGCTCGTATCTGATCTCACAGGGTTTTCATGGCTTCAACTAT | |
| desaturase | TCATTCTCCTTCTATGGAGGTCGGAAAAG | |
| Olea europaea | CTTTTCCGACCTCCATAGAAGGAGAATGAATAGTTGAAGCCATGAA | 6678 |
| Arg21Term | AACCCTGTGAGATCAGATACGAGCATCGGGGAAGGAAGGCATCTT | |
| AGA-TGA | GTGGGGTGGAAAGCAAAGTTTCAGTGCCAT | |
| | CTCGTATCTGATCTCAC | 6679 |
| | GTGAGATCAGATACGAG | 6680 |
| Increased stearate | CCCACAAGATGCCTTCCTTCCCCGATGCTCGTATCAGATCTCACAG | 6681 |
| stearoyl-ACP | GGTTTTCATGGCTTGAACTATTCATTCTCCTTCTATGGAGGTCGGA | |
| desaturase | AAAGTTAAAAAGCCTTTCACGCCTCCACG | |
| Olea europaea | CGTGGAGGCGTGAAAGGCTTTTTAACTTTTCCGACCTCCATAGAA | 6682 |
| Ser29Term | GGAGAATGAATAGTTCAAGCCATGAAAACCCTGTGAGATCTGATAC | |
| TCA-TGA | GAGCATCGGGGAAGGAAGGCATCTTGTGGG | |
| | CATGGCTTGAACTATTC | 6683 |
| | GAATAGTTCAAGCCATG | 6684 |
| Increased stearate | GATGCTCGTATCAGATCTCACAGGGTTTTCATGGCTTCAACTATTC | 6685 |
| stearoyl-ACP | ATTCTCCTTCTATGTAGGTCGGAAAAGTTAAAAAGCCTTTCACGCC | |
| desaturase | TCCACGAGAGGTACATGTTCAAGTAACCC | |
| Olea europaea | GGGTTACTTGAACATGTACCTCTCGTGGAGGCGTGAAAGGCTTTT | 6686 |
| Glu37Term | TAACTTTTCCGACCTACATAGAAGGAGAATGAATAGTTGAAGCCAT | |
| GAG-TAG | GAAAACCCTGTGAGATCTGATACGAGCATC | |
| | CTTCTATGTAGGTCGGA | 6687 |
| | TCCGACCTACATAGAAG | 6688 |
| Increased stearate | CGTATCAGATCTCACAGGGTTTTCATGGCTTCAACTATTCATTCTC | 6689 |
| stearoyl-ACP | CTTCTATGGAGGTCTGAAAAGTTAAAAAGCCTTTCACGCCTCCACG | |
| desaturase | AGAGGTACATGTTCAAGTAACCCATTCCT | |
| Olea europaea | AGGAATGGGTTACTTGAACATGTACCTCTCGTGGAGGCGTGAAAG | 6690 |
| Gly39Term | GCTTTTTAACTTTTCAGACCTCCATAGAAGGAGAATGAATAGTTGA | |
| GGA-TGA | AGCCATGAAAACCCTGTGAGATCTGATACG | |
| | TGGAGGTCTGAAAAGTT | 6691 |
| | AACTTTTCAGACCTCCA | 6692 |
| Increased stearate | TTCTCGTTTTTGTCGTCCCCTCTGCTCTCTCTCTCTATCAGGCACG | 6693 |
| stearoyl-ACP | GAGAAATGGCACTGTAACTCAGTCCAGTCATGTTTCAATCTCAGAA | |
| desaturase | GCTTCCATTTCTTGCCTCCTATCCGCCTT | |
| Persea americana | AAGGCGGATAGGAGGCAAGAAATGGAAGCTTCTGAGATTGAAACA | 6694 |
| Lys4Term | TGACTGGACTGAGTTACAGTGCCATTTCTCCGTGCCTGATAGAGA | |
| AAA-TAA | GAGAGAGCAGAGGGGACGACAAAAACGAGAA | |
| | TGGCAGTGTAACTCAGT | 6695 |
| | ACTGAGTTACAGTGCCA | 6696 |
| Increased stearate | CTGCTCTCTCTCTCTATCAGGCACGGAGAAATGGCACTGAAACTCA | 6697 |
| stearoyl-ACP | GTCCAGTCATGTTTTAATCTCAGAAGCTTCCATTTCTTGCCTCCTAT | |
| desaturase | CCGCCTTCCAATCTCAGATCTCCGAGGG | |
| Persea americana | CCCTCGGAGATCTGAGATTGGAAGGCGGATAGGAGGCAAGAAAT | 6698 |
| Gln11Term | GGAAGCTTCTGAGATTAAAACATGACTGGACTGAGTTTCAGTGCC | |
| CAA-TAA | ATTTCTCCGTGCCTGATAGAGAGAGAGAGCAG | |
| | TCATGTTTTAATCTCAG | 6699 |
| | CTGAGATTAAAACATGA | 6700 |
| Increased stearate | TCTCTCTCTATCAGGCACGGAGAAATGGCACTGAAACTCAGTCCA | 6701 |
| stearoyl-ACP | GTCATGTTTCAATCTTAGAAGCTTCCATTTCTTGCCTCCTATCCGCC | |
| desaturase | TTCCAATCTCAGATCTCCGAGGGTTTTCA | |
| Persea americana | TGAAAACCCTCGGAGATCTGAGATTGGAAGGCGGATAGGAGGCAA | 6702 |
| Gln13Term | GAAATGGAAGCTTCTAAGATTGAAACATGACTGGACTGAGTTTCAG | |
| CAG-TAG | TGCCATTTCTCCGTGCCTGATAGAGAGAGA | |
| | TTCAATCTTAGAAGCTT | 6703 |
| | AAGCTTCTAAGATTGAA | 6704 |
| Increased stearate | CTCTCTATCAGGCACGGAGAAATGGCACTGAAACTCAGTCCAGTC | 6705 |
| stearoyl-ACP | ATGTTTCAATCTCAGTAGCTTCCATTTCTTGCCTCCTATCCGCCTTC | |
| desaturase | CAATCTCAGATCTCCGAGGGTTTTCATGG | |
| Persea americana | CCATGAAAACCCTCGGAGATCTGAGATTGGAAGGCGGATAGGAG | 6706 |
| Lys14Term | GCAAGAAATGGAAGCTACTGAGATTGAAACATGACTGGACTGAGT | |
| AAG-TAG | TTCAGTGCCATTTCTCCGTGCCTGATAGAGAG | |
| | AATCTCAGTAGCTTCCA | 6707 |
| | TGGAAGCTACTGAGATT | 6708 |
| Increased stearate | CCCCGAGATCTCGCTGCCGCTGCTCATGGCGTTCGCGGCGTCCC | 6709 |
| stearoyl-ACP | ACACCGCATCGCCGTAGTCCTGCGGCGGCGTGGCGCAGAGGAG | |
| desaturase | GAGCAATGGGATGTCGAAGATGGTGGCCATGGCC | |
| Oryza sativa | GGCCATGGCCACCATCTTCGACATCCCATTGCTCCTCCTCTGCGC | 6710 |
| Tyr12Term | CACGCCGCCGCAGGACTACGGCGATGCGGTGTGGGACGCCGCG | |
| TAC-TAG | AACGCCATGAGCAGCGGCAGCGAGATCTCGGGG | |
| | TCGCCGTAGTCCTGCGG | 6711 |
| | CCGCAGGACTACGGCGA | 6712 |
| Increased stearate | CTGCTCATGGCGTTCGCGGCGTCCCACACCGCATCGCCGTACTCC | 6713 |
| stearoyl-ACP | TGCGGCGGCGTGGCGTAGAGGAGGAGCAATGGGATGTCGAAGAT | |
| desaturase | GGTGGCCATGGCCTCCACCATCAACAGGGTCA | |
| Oryza sativa | TGACCCTGTTGATGGTGGAGGCCATGGCCACCATCTTCGACATCC | 6714 |
| Gln19Term | CATTGCTCCTCCTCTACGCCACGCCGCCGCAGGAGTACGGCGAT | |
| CAG-TAG | GCGGTGTGGGACGCCGCGAACGCCATGAGCAG | |
| | GCGTGGCGTAGAGGAGG | 6715 |
| | CCTCCTCTACGCCACGC | 6716 |
| Increased stearate | CCCACACCGCATCGCCGTACTCCTGCGGCGGCGTGGCGCAGAGG | 6717 |
| stearoyl-ACP | AGGAGCAATGGGATGTAGAAGATGGTGGCCATGGCCTCCACCAT | |
| desaturase | CAACAGGGTCAAGACTGCTAAGAAGCCCTACAC | |
| Oryza sativa | GTGTAGGGCTTCTTAGCAGTCTTGACCCTGTTGATGGTGGAGGCC | 6718 |
| Ser26Term | ATGGCCACCATCTTCTACATCCCATTGCTCCTCCTCTGCGCCACGC | |
| TCG-TAG | CGCCGCAGGAGTACGGCGATGCGGTGTGGG | |
| | TGGGATGTAGAAGATGG | 6719 |
| | CCATCTTCTACATCCCA | 6720 |
| Increased stearate | CACACCGCATCGCCGTACTCCTGCGGCGGCGTGGCGCAGAGGAG | 6721 |
| stearoyl-ACP | GAGCAATGGGATGTCGTAGATGGTGGCCATGGCCTCCACCATCAA | |
| desaturase | CAGGGTCAAGACTGCTAAGAAGCCCTACACTC | |
| Oryza sativa | GAGTGTAGGGCTTCTTAGCAGTCTTGACCCTGTTGATGGTGGAGG | 6722 |
| Lys21Term | CCATGGCCACCATCTACGACATCCCATTGCTCCTCCTCTGCGCCA | |
| AAG-TAG | CGCCGCCGCAGGAGTACGGCGATGCGGTGTG | |
| | GGATGTCGTAGATGGTG | 6723 |
| | CACCATCTACGACATCC | 6724 |
| Increased stearate | TTCTCTCTCTAGGTTGAGCGGTTACCAACAGAAGCACTTAGGAGA | 6725 |
| stearoyl-ACP | GAGAAGCAATGGCGTAGAAGCTTCACCACACGGCCTTCAATCCTT | |
| desaturase | CCATGGCGGTTACCTCTTCGGGACTTCCTCG | |
| Simmondsia chinensis | CGAGGAAGTCCCGAAGAGGTAACCGCCATGGAAGGATTGAAGGC | 6726 |
| Leu3Term | CGTGTGGTGAAGCTTCTACGCCATTGCTTCTCTCTCCTAAGTGCTT | |
| TTG-TAG | CTGTTGGTAACCGCTCAACCTAGAGAGAGAA | |
| | AATGGCGTAGAAGCTTC | 6727 |
| | GAAGCTTCTACGCCATT | 6728 |
| Increased stearate | CTCTCTCTAGGTTGAGCGGTTACCAACAGAAGCACTTAGGAGAGA | 6729 |
| stearoyl-ACP | GAAGCAATGGCGTTGTAGCTTCACCACACGGCCTTCAATCCTTCC | |
| desaturase | ATGGCGGTTACCTCTTCGGGACTTCCTCGAT | |
| Simmondsia chinensis | ATCGAGGAAGTCCCGAAGAGGTAACCGCCATGGAAGGATTGAAG | 6730 |
| Lys4Term | GCCGTGTGGTGAAGCTACAACGCCATTGCTTCTCTCTCCTAAGTG | |
| AAG-TAG | CTTCTGTTGGTAACCGCTCAACCTAGAGAGAG | |
| | TGGCGTTGTAGCTTCAC | 6731 |
| | GTGAAGCTACAACGCCA | 6732 |
| Increased stearate | AAGCAATGGCGTTGAAGCTTCACCACACGGCCTTCAATCCTTCCAT | 6733 |
| stearoyl-ACP | GGCGGTTACCTCTTAGGGACTTCCTCGATCGTATCACCTCAGATCT | |
| desaturase | CACCGCGTTTTCATGGCTTCTTCTACAAT | |
| Simmondsia chinensis | ATTGTAGAAGAAGCCATGAAAACGCGGTGAGATCTGAGGTGATAC | 6734 |
| Ser19Term | GATCGAGGAAGTCCCTAAGAGGTAACCGCCATGGAAGGATTGAAG | |
| TCG-TAG | GCCGTGTGGTGAAGCTTCAACGCCATTGCTT | |
| | TACCTCTTAGGGACTTC | 6735 |
| | GAAGTCCCTAAGAGGTA | 6736 |
| Increased stearate | GCAATGGCGTTGAAGCTTCACCACACGGCCTTCAATCCTTCCATG | 6737 |
| stearoyl-ACP | GCGGTTACCTCTTCGTGACTTCCTCGATCGTATCACCTCAGATCTC | |
| desaturase | ACCGCGTTTTCATGGCTTCTTCTACAATTG | |
| Simmondsia chinensis | CAATTGTAGAAGAAGCCATGAAAACGCGGTGAGATCTGAGGTGAT | 6738 |
| Gly20Term | ACGATCGAGGAAGTCACGAAGAGGTAACCGCCATGGAAGGATTG | |
| GGA-TGA | AAGGCCGTGTGGTGAAGCTTCAACGCCATTGC | |
| | CCTCTTCGTGACTTCCT | 6739 |
| | AGGAAGTCACGAAGAGG | 6740 |
| Increased stearate | TGGCTCTGAATCTCAACCCCGTTTCCACACCATTTCAGTGTCGTCG | 6741 |
| stearoyl-ACP | ATTGCCGTCTTTCTGACCTCGTCAAACGCCTTCTCGCAGATCTCCC | |
| desaturase | AAATTCTTCATGGCTTCCACTCTCAGCAG | |
| Spinacia oleracea | CTGCTGAGAGTGGAAGCCATGAAGAATTTGGGAGATCTGCGAGAA | 6742 |
| Ser21Term | GGCGTTTGACGAGGTCAGAAAGACGGCAATCGACGACACTGAAAT | |
| TCA-TGA | GGTGTGGAAACGGGGTTGAGATTCAGAGCCA | |
| | GTCTTTCTGACCTCGTC | 6743 |
| | GACGAGGTCAGAAAGAC | 6744 |
| Increased stearate | AATCTCAACCCCGTTTCCACACCATTTCAGTGTCGTCGATTGCCGT | 6745 |
| stearoyl-ACP | CTTTCTCACCTCGTTAAACGCCTTCTCGCAGATCTCCCAAATTCTT | |
| desaturase | CATGGCTTCCACTCTCAGCAGCTCTTCTC | |
| Spinacia oleracea | GAGAAGAGCTGCTGAGAGTGGAAGCCATGAAGAATTTGGGAGATC | 6746 |
| Gln24Term | TGCGAGAAGGCGTTTAACGAGGTGAGAAAGACGGCAATCGACGA | |
| CAA-TAA | CACTGAAATGGTGTGGAAACGGGGTTGAGATT | |
| | CACCTCGTTAAACGCCT | 6747 |
| | AGGCGTTTAACGAGGTG | 6748 |
| Increased stearate | TCCACACCATTTCAGTGTCGTCGATTGCCGTCTTTCTCACCTCGTC | 6749 |
| stearoyl-ACP | AAACGCCTTCTCGCTGATCTCCCAAATTCTTCATGGCTTCCACTCT | |
| desaturase | CAGCAGCTCTTCTCCTAAGGAAGCGGAAA | |
| Spinacia oleracea | TTTCCGCTTCCTTAGGAGAAGAGCTGCTGAGAGTGGAAGCCATGA | 6750 |
| Arg29Term | AGAATTTGGGAGATCAGCGAGAAGGCGTTTGACGAGGTGAGAAA | |
| AGA-TGA | GACGGCAATCGACGACACTGAAATGGTGTGGA | |
| | CTTCTCGCTGATCTCCC | 6751 |
| | GGGAGATCAGCGAGAAG | 6752 |
| Increased stearate | TTTCAGTGTCGTCGATTGCCGTCTTTCTCACCTCGTCAAACGCCTT | 6753 |
| stearoyl-ACP | CTCGCAGATCTCCCTAATTCTTCATGGCTTCCACTCTCAGCAGCTC | |
| desaturase | TTCTCCTAAGGAAGCGGAAAGCCTGAAGA | |
| Spinacia oleracea | TCTTCAGGCTTTCCGCTTCCTTAGGAGAAGAGCTGCTGAGAGTGG | 6754 |
| Lys32Term | AAGCCATGAAGAATTAGGGAGATCTGCGAGAAGGCGTTTGACGAG | |
| AAA-TAA | GTGAGAAAGACGGCAATCGACGACACTGAAA | |
| | GATCTCCCTAATTCTTC | 6755 |
| | GAAGAATTAGGGAGATC | 6756 |
| Increased stearate | AAATAGTCGAGGTGAAAAACAGAGCATCAACAATGGCACTGAATAT | 6757 |
| stearoyl-ACP | CAATGGGGTGTCGTGAAAATCTCACAAAATGTTACCATTTCCTTGT | |
| desaturase | TCTTCAGCCAGATCTGAGCGAGTTTTCAT | |
| Solanum tuberosum | ATGAAAACTCGCTCAGATCTGGCTGAAGAACAAGGAAATGGTAAC | 6758 |
| Leu10Term | ATTTTGTGAGATTTTCACGACACCCCATTGATATTCAGTGCCATTGT | |
| TTA-TGA | TGATGCTCTGTTTTTCACCTCGACTATTT | |
| | GGTGTCGTGAAAATCTC | 6759 |
| | GAGATTTTCACGACACC | 6760 |
| Increased stearate | ATAGTCGAGGTGAAAAACAGAGCATCAACAATGGCACTGAATATCA | 6761 |
| stearoyl-ACP | ATGGGGTGTCGTTATAATCTCACAAAATGTTACCATTTCCTTGTTCT | |
| desaturase | TCAGCCAGATCTGAGCGAGTTTTCATGG | |
| Solanum tuberosum | CCATGAAAACTCGCTCAGATCTGGCTGAAGAACAAGGAAATGGTA | 6762 |
| Lys11Term | ACATTTTGTGAGATTATAACGACACCCCATTGATATTCAGTGCCATT | |
| AAA-TAA | GTTGATGCTCTGTTTTTCACCTCGACTAT | |
| | TGTCGTTATAATCTCAC | 6763 |
| | GTGAGATTATAACGACA | 6764 |
| Increased stearate | GTGAAAAACAGAGCATCAACAATGGCACTGAATATCAATGGGGTG | 6765 |
| stearoyl-ACP | TCGTTAAAATCTCACTAAATGTTACCATTTCCTTGTTCTTCAGCCAG | |
| desaturase | ATCTGAGCGAGTTTTCATGGCTTCAACCA | |
| Solanum tuberosum | TGGTTGAAGCCATGAAAACTCGCTCAGATCTGGCTGAAGAACAAG | 6766 |
| Lys14Term | GAAATGGTAACATTTAGTGAGATTTTAACGACACCCCATTGATATT | |
| AAA-TAA | CAGTGCCATTGTTGATGCTCTGTTTTTCAC | |
| | AATCTCACTAAATGTTA | 6767 |
| | TAACATTTAGTGAGATT | 6768 |
| Increased stearate | ACAGAGCATCAACAATGGCACTGAATATCAATGGGGTGTCGTTAAA | 6769 |
| stearoyl-ACP | ATCTCACAAAATGTGACCATTTCCTTGTTCTTCAGCCAGATCTGAG | |
| desaturase | CGAGTTTTCATGGCTTCAACCATTCATCG | |
| Solanum tuberosum | CGATGAATGGTTGAAGCCATGAAAACTCGCTCAGATCTGGCTGAA | 6770 |
| Leu16Term | GAACAAGGAAATGGTCACATTTTGTGAGATTTTAACGACACCCCAT | |
| TTA-TGA | TGATATTCAGTGCCATTGTTGATGCTCTGT | |
| | CAAAATGTGACCATTTC | 6771 |
| | GAAATGGTCACATTTTG | 6772 |
| Increased stearate | TGGCTCTGAGGCTGAACCCTAACCCTTCACAGAAGCTCTTTCTCTC | 6773 |
| stearoyl-ACP | TCCTTCTTCATCATGATCTTCTTCTTCTTCATCGTTCTCGCTTCCTC | |
| desaturase | AAATGGCTAGCCTCAGATCTCCAAGGTT | |
| Arachis hypogaea | AACCTTGGAGATCTGAGGCTAGCCATTTGAGGAAGCGAGAACGAT | 6774 |
| Ser21Term | GAAGAAGAAGAAGATCATGATGAAGAAGGAGAGAGAAAGAGCTTC | |
| TCA-TGA | TGTGAAGGGTTAGGGTTCAGCCTCAGAGCCA | |
| | TTCATCATGATCTTCTT | 6775 |
| | AAGAAGATCATGATGAA | 6776 |
| Increased stearate | ACCCTAACCCTTCACAGAAGCTCTTTCTCTCTCCTTCTTCATCATCA | 6777 |
| stearoyl-ACP | TCTTCTTCTTCTTGATCGTTCTCGCTTCCTCAAATGGCTAGCCTCA | |
| desaturase | GATCTCCAAGGTTCCGCATGGCCTCCAC | |
| Arachis hypogaea | GTGGAGGCCATGCGGAACCTTGGAGATCTGAGGCTAGCCATTTGA | 6778 |
| Ser26Term | GGAAGCGAGAACGATCAAGAAGAAGAAGATGATGATGAAGAAGGA | |
| TCA-TGA | GAGAGAAAGAGCTTCTGTGAAGGGTTAGGGT | |
| | TTCTTCTTGATCGTTCT | 6779 |
| | AGAACGATCAAGAAGAA | 6780 |
| Increased stearate | CTAACCCTTCACAGAAGCTCTTTCTCTCTCCTTCTTCATCATCATCT | 6781 |
| stearoyl-ACP | TCTTCTTCTTCATAGTTCTCGCTTCCTCAAATGGCTAGCCTCAGAT | |
| desaturase | CTCCAAGGTTCCGCATGGCCTCCACCCT | |
| Arachis hypogaea | AGGGTGGAGGCCATGCGGAACCTTGGAGATCTGAGGCTAGCCAT | 6782 |
| Ser27Term | TTGAGGAAGCGAGAACTATGAAGAAGAAGAAGATGATGATGAAGA | |
| TCG-TAG | AGGAGAGAGAAAGAGCTTCTGTGAAGGGTTAG | |
| | TTCTTCATAGTTCTCGC | 6783 |
| | GCGAGAACTATGAAGAA | 6784 |
| Increased stearate | CTTCACAGAAGCTCTTTCTCTCTCCTTCTTCATCATCATCTTCTTCT | 6785 |
| stearoyl-ACP | TCTTCATCGTTCTAGCTTCCTCAAATGGCTAGCCTCAGATCTCCAA | |
| desaturase | GGTTCCGCATGGCCTCCACCCTCCGCAC | |
| Arachis hypogaea | GTGCGGAGGGTGGAGGCCATGCGGAACCTTGGAGATCTGAGGCT | 6786 |
| Ser29Term | AGCCATTTGAGGAAGCTAGAACGATGAAGAAGAAGAAGATGATGA | |
| TCG-TAG | TGAAGAAGGAGAGAGAAAGAGCTTCTGTGAAG | |
| | ATCGTTCTAGCTTCCTC | 6787 |
| | GAGGAAGCTAGAACGAT | 6788 |
| Increased stearate | AAAGTTAAAAGCCGTCCAAAACCCAAACCAGGAAAGGCAAACGAA | |
| stearoyl-ACP | AAGAAAAAATGGCTTAGAATTTTAATGCCATCGCCTCGAAATCTCA | |
| desaturase | GAAGCTCCCTTGCTTTGCTCTTCCACCAAA | |
| Gossypium hirsutum | TTTGGTGGAAGAGCAAAGCAAGGGAGCTTCTGAGATTTCGAGGCG | 6790 |
| Leu3Term | ATGGCATTAAAATTCTAAGCCATTTTTTCTTTTCGTTTGCCTTTCCT | |
| TTG-TAG | GGTTTGGGTTTTGGACGGCTTTTAACTTT | |
| | AATGGCTTAGAATTTTA | 6791 |
| | TAAAATTCTAAGCCATT | 6792 |
| Increased stearate | CCCAAACCAGGAAAGGCAAACGAAAAGAAAAAATGGCTTTGAATTT | 6793 |
| stearoyl-ACP | TAATGCCATCGCCTAGAAATCTCAGAAGCTCCCTTGCTTTGCTCTT | |
| desaturase | CCACCAAAGGCCACCCTTAGATCTCCCAA | |
| Gossypium hirsutum | TTGGGAGATCTAAGGGTGGCCTTTGGTGGAAGAGCAAAGCAAGG | 6794 |
| Ser1Term | GAGCTTCTGAGATTTCTAGGCGATGGCATTAAAATTCAAAGCCATT | |
| TCG-TAG | TTTTCTTTTCGTTTGCCTTTCCTGGTTTGGG | |
| | CATCGCCTAGAAATCTC | 6795 |
| | GAGATTTCTAGGCGATG | 6796 |
| Increased stearate | CAAACCAGGAAAGGCAAACGAAAAGAAAAAATGGCTTTGAATTTTA | 6797 |
| stearoyl-ACP | ATGCGATCGCCTCGTAATCTCAGAAGCTCCCTTGCTTTGCTCTTCC | |
| desaturase | ACCAAAGGCCACCCTTAGATCTCCCAAGT | |
| Gossypium hirsutum | ACTTGGGAGATCTAAGGGTGGCCTTTGGTGGAAGAGCAAAGCAAG | 6798 |
| Lys11Term | GGAGCTTCTGAGATTACGAGGCGATGGCATTAAAATTCAAAGCCA | |
| AAA-TAA | TTTTTTCTTTTCGTTTGCCTTTCCTGGTTTG | |
| | TCGCCTCGTAATCTCAG | 6799 |
| | CTGAGATTACGAGGCGA | 6800 |
| Increased stearate | AGGAAAGGCAAACGAAAAGAAAAAATGGCTTTGAATTTTAATGCCA | 6801 |
| stearoyl-ACP | TCGCCTCGAAATCTTAGAAGCTCCCTTGCTTTGCTCTTCCACCAAA | |
| desaturase | GGCCACCCTTAGATCTCCCAAGTTTTCCA | |
| Gossypium hirsutum | TGGAAAACTTGGGAGATCTAAGGGTGGCCTTTGGTGGAAGAGCAA | 6802 |
| Gln13Term | AGCAAGGGAGCTTCTAAGATTTCGAGGCGATGGCATTAAAATTCA | |
| CAG-TAG | AAGCCATTTTTTCTTTTCGTTTGCCTTTCCT | |
| | CGAAATCTTAGAAGCTC | 6803 |
| | GAGCTTCTAAGATTTCG | 6804 |

TABLE 42
Oligonucleotides to produce plants with reduced linolenic acid

| Phenotype, Gene, | | |
|---|---|---|
| Plant & Targeted | | SEQ ID |
| Alteration | Altering Oligos | NO: |
| Reduced linolenic acid | AATAGAACGACAGAGACTTTTTCCTCTTTTCTTCTTGGGAAGAGGC | 6805 |
| omega-3 fatty acid | TCCAATGGCGAGCTAGGTTTTATCAGAATGTGGTTTTAGACCTCTC | |
| desaturase | CCCAGATTCTACCCTAAACACACAACCTC | |
| Arabidopsis thaliana | GAGGTTGTGTGTTTAGGGTAGAATCTGGGGAGAGGTCTATAAACCA | 6806 |
| Ser4Term | CATTCTGATAAAACCTAGCTCGCCATTGGAGCCTCTTCCCAAGAAG | |
| TCG-TAG | AAAAGAGGAAAAAGTCTCTGTCGTTCTATT | |
| | GGCGAGCTTGGTTTTAT | 6807 |
| | ATAAAACCAAGCTCGCC | 6808 |
| Reduced linolenic acid | ACGACAGAGACTTTTTCCTCTTTTCTTCTTGGGAAGAGGCTCCAAT | 6809 |
| omega-3 fatty acid | GGCGAGCTCGGTTTGATCAGAATGTGGTTTTAGACCTCTCCCCAG | |
| desaturase | ATTCTACCCTAAACACACAACCTCTTTTGC | |
| Arabidopsis thaliana | GCAAAAGAGGTTGTGTGTTTAGGGTAGAATCTGGGGAGAGGTCTA | 6810 |
| Leu6Term | AAACCACATTCTGATCAAACCGAGCTCGCCATTGGAGCCTCTTCCC | |
| TTA-TGA | AAGAAGAAAAGAGGAAAAAGTCTCTGTCGT | |
| | CTCGGTTTGATCAGAAT | 6811 |
| | ATTCTGATCAAACCGAG | 6812 |
| Reduced linolenic acid | ACAGAGACTTTTTCCTCTTTTCTTCTTGGGAAGAGGCTCCAATGGC | 6813 |
| omega-3 fatty acid | GAGCTCGGTTTTATGAGAATGTGGTTTTAGACCTCTCCCCAGATTC | |
| desaturase | TACCCTAAACACACAACCTCTTTTGCCTC | |
| Arabidopsis thaliana | GAGGCAAAAGAGGTTGTGTGTTTAGGGTAGAATCTGGGGAGAGGT | 6814 |
| Ser7Term | CTAAAACCACATTCTCATAAAACCGAGCTCGCCATTGGAGCCTCTT | |
| TCA-TGA | CCCAAGAAGAAAAGAGGAAAAAGTCTCTGT | |
| | GGTTTTATGAGAATGTG | 6815 |
| | CACATTCTCATAAAACC | 6816 |
| Reduced linolenic acid | AGAGACTTTTTCCTCTTTTCTTCTTGGGAAGAGGCTCCAATGGCGA | 6817 |
| omega-3 fatty acid | GCTCGGTTTTATCATAATGTGGTTTTAGACCTCTCCCCAGATTCTA | |
| desaturase | CCCTAAACACACAACCTCTTTTGCCTCTA | |
| Arabidopsis thaliana | TAGAGGCAAAAGAGGTTGTGTGTTTAGGGTAGAATCTGGGGAGAG | 6818 |
| Glu8Term | GTCTAAAACCACATTATGATAAAACCGAGCTCGCCATTGGAGCCTC | |
| GAA-TAA | TTCCCAAGAAGAAAAGAGGAAAAAGTCTCT | |
| | TTTTATCATAATGTGGT | 6819 |
| | ACCACATTATGATAAAA | 6820 |
| Reduced linolenic acid | CATCATCTTCTTCTTCTGGGGAGAGAGAGAGAGCAAAAGAGCTC | 6821 |
| omega-3 fatty acid | TAGCAATGGCGAACTAGGTCTTATCCGAATGTGGCATAAGACCTC | |
| desaturase | TCCCCAGAATCTACACCACACCCAGATCCAC | |
| Brassica juncea | GTGGATCTGGGTGTGGTGTAGATTCTGGGGAGAGGTCTTATGCCA | 6822 |
| Leu4Term | CATTCGGATAAGACCTAGTTCGCCATTGCTAGAGCTCTTTTGCTCT | |
| TTG-TAG | CTCTCTCTCCCCAGAAGAAGAAGATGATGA | |
| | GGCGAACTAGGTCTTAT | 6823 |
| | ATAAGACCTAGTTCGCC | 6824 |
| Reduced linolenic acid | TCTTCTTCTTCTGGGGAGAGAGAGAGAGCAAAAGAGCTCTAGCAA | 6825 |
| omega-3 fatty acid | TGGCGAACTTGGTCTGATCCGAATGTGGCATAAGACCTCTCCCGA | |
| desaturase | GAATCTACACCACACCCAGATCCACTTTCCT | |
| Brassica juncea | AGGAAAGTGGATCTGGGTGTGGTGTAGATTCTGGGGAGAGGTCTT | 6826 |
| Leu6Term | ATGCCACATTCGGATCAGACCAAGTTCGCCATTGCTAGAGCTCTTT | |
| TTA-TGA | TGCTCTCTCTCTCTCCCCAGAAGAAGAAGA | |
| | CTTGGTCTGATCCGAAT | 6827 |
| | ATTCGGATCAGACCAAG | 6828 |
| Reduced malelic acid | TTCTTCTGGGGAGAGAGAGAGAGCAAAAGAGCTCTAGCAATGGCG | 6829 |
| omega-3 fatty acid | AACTTGGTCTTATCCTAATGTGGCATAAGACCTCTCCCCAGAATCT | |
| desaturase | ACACCACACCCAGATCCACTTTCCTCTCCA | |
| Brassica juncea | TGGAGAGGAAAGTGGATCTGGGTGTGGTGTAGATTCTGGGGAGA | 6830 |
| Glu8Term | GGTCTTATGCCACATTAGGATAAGACCAAGTTCGCCATTGCTAGA | |
| GAA-TAA | GCTCTTTTGCTCTCTCTCTCTCCCCAGAAGAA | |
| | TCTTATCCTAATGTGGC | 6831 |
| | GCCACATTAGGATAAGA | 6832 |
| Reduced linolenic acid | CTGGGGAGAGAGAGAGAGCAAAAGAGCTCTAGCAATGGCGAACT | 6833 |
| omega-3 fatty acid | TGGTCTTATCCGAATGAGGCATAAGACCTCTCCCCAGAATCTACAC | |
| desaturase | CACACCCAGATCCACTTTCCTCTCCAACACC | |
| Brassica juncea | GGTGTTGGAGAGGAAAGTGGATCTGGGTGTGGTGTAGATTCTGG | 6834 |
| Cys9Term | GGAGAGGTCTTATGCCTCATTCGGATAAGACCAAGTTCGCCATTG | |
| TGT-TGA | CTAGAGCTCTTTTGCTCTCTCTCTCTCCCCAG | |
| | TCCGAATGAGGCATAAG | 6835 |
| | CTTATGCCTCATTCGGA | 6836 |
| Reduced linolenic acid | ATAACAGAATTGCTGAATTCTTGCATTTTTAGCTTCTGGGTTTTCAA | 6837 |
| omega-3 fatty acid | TGGCTGCTGGTTGAGTATTATCAGAATGTGGTTTAAGGCCTCTCCC | |
| desaturase | AAGAATCTACTCACGACCCAGAATTGGT | |
| Ricinus communis | ACCAATTCTGGGTCGTGAGTAGATTCTTGGGAGAGGCCTTAAACC | 6838 |
| Trp5Term | ACATTCTGATAATACTCAACCAGCAGCCATTGAAAACCCAGAAGCT | |
| TGG-TGA | AAAAATGCAAGAATTCAGCAATTCTGTTAT | |
| | GCTGGTTGAGTATTATC | 6839 |
| | GATAATACTCAACCAGC | 6840 |
| Reduced linolenic acid | AGAATTGCTGAATTCTTGCATTTTTAGCTTCTGGGTTTTCAATGGCT | 6841 |
| omega-3 fatty acid | GCTGGTTGGGTATGATCAGAATGTGGTTTAAGGCCTCTCCCAAGA | |
| desaturase | ATCTACTCACGACCCAGAATTGGTTTTAC | |
| Ricinus communis | GTAAAACCAATTCTGGGTCGTGAGTAGATTCTTGGGAGAGGCCTT | 6842 |
| Leu7Term | AAACCACATTCTGATCATACCCAACCAGCAGCCATTGAAAACCCAG | |
| TTA-TGA | AAGCTAAAAATGCAAGAATTCAGCAATTCT | |
| | TTGGGTATGATCAGAAT | 6843 |
| | ATTCTGATCATACCCAA | 6844 |
| Reduced linolenic acid | ATTGCTGAATTCTTGCATTTTTAGCTTCTGGGTTTTCAATGGCTGCT | 6845 |
| omega-3 fatty acid | GGTTGGGTATTATGAGAATGTGGTTTAAGGCCTCTCCCAAGAATCT | |
| desaturase | ACTCACGACCCAGAATTGGTTTTACATC | |
| Ricinus communis | GATGTAAAACCAATTCTGGGTCGTGAGTAGATTCTTGGGAGAGGC | 6846 |
| Ser8Term | CTTAAACCACATTCTCATAATACCCAACCAGCAGCCATTGAAAACC | |
| TCA-TGA | CAGAAGCTAAAAATGCAAGAATTCAGCAAT | |
| | GGTATTATGAGAATGTG | 6847 |
| | CACATTCTCATAATACC | 6848 |
| Reduced linolenic acid | TGCTGAATTCTTGCATTTTTAGCTTCTGGGTTTTCAATGGCTGCTG | 6849 |
| omega-3 fatty acid | GTTGGGTATTATCATAATGTGGTTTAAGGCCTCTCCCAAGAATCTA | |
| desaturase | CTCACGACCCAGAATTGGTTTTACATCGA | |
| Ricinus communis | TCGATGTAAAACCAATTCTGGGTCGTGAGTAGATTCTTGGGAGAG | 6850 |
| Glu9Term | GCCTTAAACCACATTATGATAATACCCAACCAGCAGCCATTGAAAA | |
| GAA-TAA | CCCAGAAGCTAAAAATGCAAGAATTCAGCA | |
| | TATTATCATAATGTGGT | 6851 |
| | ACCACATTATGATAATA | 6852 |
| Reduced linolenic acid | GCAAGTTGGGTTTTATCAGAATGTGGTCTTAGACCACTCCCAAGAA | 6853 |
| omega-3 fatty acid | TCTACGCTAAGCCCTGAACTGGGGCAGCCACTTCTGCCTCCTCTC | |
| desaturase | ACATTAAGTTGAGAATTTCACGTACAGATC | |
| Nicotiana tabacum | GATCTGTACGTGAAATTCTCAACTTAATGTGAGAGGAGGCAGAAGT | 6854 |
| Arg22Term | GGCTGCCCCAGTTCAGGGCTTAGGGTAGATTCTTGGGAGTGGTCT | |
| AGA-TGA | AAGACCACATTCTGATAAAACCCAACTTGC | |
| | CTAAGCCCTGAACTGGG | 6855 |
| | CCCAGTTCAGGGCTTAG | 6856 |
| Reduced linolenic acid | CTCCCAAGAATCTACCCTAAGCCCAGAACTGGGGCAGCCACTTCT | 6857 |
| omega-3 fatty acid | GCCTCCTCTCACATTTAGTTGAGAATTTCACGTACAGATCTGAGTG | |
| desaturase | GTTCTGCAATTTCTTTGTCTAATACTAATA | |
| Nicotiana tabacum | TATTAGTATTAGACAAAGAAATTGCAGAACCACTCAGATCTGTACG | 6858 |
| Lys34Term | TGAAATTCTCAACTAAATGTGAGAGGAGGCAGAAGTGGCTGCCCC | |
| AAG-TAG | AGTTCTGGGCTTAGGGTAGATTCTTGGGAG | |
| | CTCACATTTAGTTGAGA | 6859 |
| | TCTCAACTAAATGTGAG | 6860 |
| Reduced malefic acid | CAAGAATCTACCCTAAGCCCAGAACTGGGGCAGCCACTTCTGCCT | 6861 |
| omega-3 fatty acid | CCTCTCACATTAAGTAGAGAATTTCACGTACAGATCTGAGTGGTTC | |
| desaturase | TGCAATTTCTTTGTCTAATACTAATAAAGA | |
| Nicotiana tabacum | TCTTTATTAGTATTAGACAAAGAAATTGCAGAACCACTCAGATCTGT | 6862 |
| Leu35Term | ACGTGAAATTCTCTACTTAATGTGAGAGGAGGCAGAAGTGGCTGC | |
| TTG-TAG | CCCAGTTCTGGGCTTAGGGTAGATTCTTG | |
| | CATTAAGTAGAGAATTT | 6863 |
| | AAATTCTCTACTTAATG | 6864 |
| Reduced linolenic acid | AGAATCTACCCTAAGCCCAGAACTGGGGCAGCCACTTCTGCCTCC | 6865 |
| omega-3 fatty acid | TCTCACATTAAGTTGTGAATTTCACGTACAGATCTGAGTGGTTCTG | |
| desaturase | CAATTTCTTTGTCTAATACTAATAAAGAGA | |
| Nicotiana tabacum | TCTCTTTATTAGTATTAGACAAAGAAATTGCAGAACCACTCAGATCT | 6866 |
| Arg36Term | GTACGTGAAATTCACAACTTAATGTGAGAGGAGGCAGAAGTGGCT | |
| AGA-TGA | GCCCCAGTTCTGGGCTTAGGGTAGATTCT | |
| | TTAAGTTGTGAATTTCA | 6867 |
| | TGAAATTCACAACTTAA | 6868 |
| Reduced linolenic acid | GCGAGTTGGGTTTTATCAGAATGTGGTCTGAGGCCACTCCCGAGG | 6869 |
| omega-3 fatty acid | GTCTATCCTAAGCCATGAACTGGCCACCCTTTGTTGAATTCCAATC | |
| desaturase | CCACAAAGCTGAGATTTTCAAGAACAGATC | |
| Sesamum indicum | GATCTGTTCTTGAAAATCTCAGCTTTGTGGGATTGGAATTCAACAA | 6870 |
| Arg22Term | AGGGTGGCCAGTTCATGGCTTAGGATAGACCCTCGGGAGTGGCC | |
| AGA-TGA | TCAGACCACATTCTGATAAAACCCAACTCGC | |
| | CTAAGCCATGAACTGGC | 6871 |
| | GCCAGTTCATGGCTTAG | 6872 |
| Reduced linolenic acid | CAGAATGTGGTCTGAGGCCACTCCCGAGGGTCTATCCTAAGCCAA | 6873 |
| omega-3 fatty acid | GAACTGGCCACCCTTAGTTGAATTCCAATCCCACAAAGCTGAGATT | |
| desaturase | TTCAAGAACAGATCTTGGAAATGGTTCTTC | |
| Sesamum indicum | GAAGAACCATTTCCAAGATCTGTTCTTGAAAATCTCAGCTTTGTGG | 6874 |
| Leu21Term | GATTGGAATTCAACTAAGGGTGGCCAGTTCTTGGCTTAGGATAGA | |
| TTG-TAG | CCCTCGGGAGTGGCCTCAGACCACATTCTG | |
| | CCACCCTTAGTTGAATT | 6875 |
| | AATTCAACTAAGGGTGG | 6876 |
| Reduced linolenic acid | AATGTGGTCTGAGGCCACTCCCGAGGGTCTATCCTAAGCCAAGAA | 6877 |
| omega-3 fatty acid | CTGGCCACCCTTTGTAGAATTCCAATCCCACAAAGCTGAGATTTTC | |
| desaturase | AAGAACAGATCTTGGAAATGGTTCTTCATT | |
| Sesamum indicum | AATGAAGAACCATTTCCAAGATCTGTTCTTGAAAATCTCAGCTTTGT | 6878 |
| Leu28Term | GGGATTGGAATTCTACAAAGGGTGGCCAGTTCTTGGCTTAGGATA | |
| TTG-TAG | GACCCTCGGGAGTGGCCTCAGACCACATT | |
| | CCCTTTGTAGAATTCCA | 6879 |
| | TGGAATTCTACAAAGGG | 6880 |
| Reduced linolenic acid | CTCCCGAGGGTCTATCCTAAGCCAAGAACTGGCCACCCTTTGTTG | 6881 |
| omega-3 fatty acid | AATTCCAATCCCACATAGCTGAGATTTTCAAGAACAGATCTTGGAA | |
| desaturase | ATGGTTCTTCATTCTGTTTGTCGAGTGGGA | |
| Sesamum indicum | TCCCACTCGACAAACAGAATGAAGAACCATTTCCAAGATCTGTTCT | 6882 |
| Lys34Term | TGAAAATCTCAGCTATGTGGGATTGGAATTCAACAAAGGGTGGCC | |
| AAG-TAG | AGTTCTTGGCTTAGGATAGACCCTCGGGAG | |
| | ATCCCACATAGCTGAGA | 6883 |
| | TCTCAGCTATGTGGGAT | 6884 |
| Reduced linolenic acid | CATCAGAGCGGGGATACCTAAGCATTGCTGGGTTAAGAATCCATG | 6885 |
| omega-3 fatty acid | GAAGTCTATGAGTTAGGTCGTCAGAGAGCTAGCCATCGTGTTCGC | |
| desaturase | ACTAGCTGCTGGAGCTGCTTACCTCAACAAT | |
| Brassica napus | ATTGTTGAGGTAAGCAGCTCCAGCAGCTAGTGCGAACACGATGGC | 6886 |
| Tyr3Term | TAGCTCTCTGACGACCTAACTCATAGACTTCCATGGATTCTTAACC | |
| TAC-TAG | CAGCAATGCTTAGGTATCGCCGCTCTGATG | |
| | ATGAGTTAGGTCGTCAG | 6887 |
| | CTGACGACCTAACTCAT | 6888 |
| Reduced linolenic acid | GCGGCGATACCTAAGCATTGCTGGGTTAAGAATCCATGGAAGTCT | 6889 |
| omega-3 fatty acid | ATGAGTTACGTCGTCTGAGAGCTAGCCATCGTGTTCGCACTAGCT | |
| desaturase | GCTGGAGCTGCTTACCTCAACAATTGGCTTG | |
| Brassica napus | CAAGCCAATTGTTGAGGTAAGCAGCTCCAGCAGCTAGTGCGAACA | 6890 |
| Arg6Term | CGATGGCTAGCTCTCAGACGACGTAACTCATAGACTTCCATGGATT | |
| AGA-TGA | CTTAACCCAGCAATGCTTAGGTATCGCCGC | |
| | ACGTCGTCTGAGAGCTA | 6891 |
| | TAGCTCTCAGACGACGT | 6892 |
| Reduced linolenic acid | GCGATACCTAAGCATTGCTGGGTTAAGAATCCATGGAAGTCTATGA | 6893 |
| omega-3 fatty acid | GTTACGTCGTCAGATAGCTAGCCATCGTGTTCGCACTAGCTGCTG | |
| desaturase | GAGCTGCTTACCTCAACAATTGGCTTGTTT | |
| Brassica napus | AAACAAGCCAATTGTTGAGGTAAGCAGCTCCAGCAGCTAGTGCGA | 6894 |
| Glu7Term | ACACGATGGCTAGCTATCTGACGACGTAACTCATAGACTTCCATG | |
| GAG-TAG | GATTCTTAACCCAGCAATGCTTAGGTATCGC | |
| | TCGTCAGATAGCTAGCC | 6895 |
| | GGCTAGCTATCTGACGA | 6896 |
| Reduced linolenic acid | CCATGGAAGTCTATGAGTTACGTCGTCAGAGAGCTAGCCATCGTG | 6897 |
| omega-3 fatty acid | TTCGCACTAGCTGCTTGAGCTGCTTACCTCAACAATTGGCTTGTTT | |
| desaturase | GGCCTCTCTATTGGATTGCTCAAGGAACCA | |
| Brassica napus | TGGTTCCTTGAGCAATCCAATAGAGAGGCCAAACAAGCCAATTGTT | 6898 |
| Gly17Term | GAGGTAAGCAGCTCAAGCAGCTAGTGCGAACACGATGGCTAGCT | |
| GGA-TGA | CTCTGACGACGTAACTCATAGACTTCCATGG | |
| | TAGCTGCTTGAGCTGCT | 6899 |
| | AGCAGCTCAAGCAGCTA | 6900 |
| Reduced linolenic acid | GCAAGTTGGGTTCTATCAGAATGTGGTCTTAGACCACTACCAAGAA | 6901 |
| omega-3 fatty acid | TATACCCAAAGCCCTGAATAGGGTCTTCTTCCGTTTGCGCCACCAA | |
| desaturase | TTTAAATCTGAGAAGAATTTCACCTTCAC | |
| Solanum tuberosum | GTGAAGGTGAAATTCTTCTCAGATTTAAATTGGTGGCGCAAACGGA | 6902 |
| Arg22Term | AGAAGACCCTATTCAGGGCTTTGGGTATATTCTTGGTAGTGGTCTA | |
| AGA-TGA | AGACCACATTCTGATAGAACCCAACTTGC | |
| | CAAAGCCCTGAATAGGG | 6903 |
| | CCCTATTCAGGGCTTTG | 6904 |
| Reduced linolenic acid | TGGTCTTAGACCACTACCAAGAATATACCCAAAGCCCAGAATAGG | 6905 |
| omega-3 fatty acid | GTCTTCTTCCGTTTGAGCCACCAATTTAAATCTGAGAAGAATTTCA | |
| desaturase | CCTTCACCTATACGAACAGATCGGAATTGT | |
| Solanum tuberosum | ACAATTCCGATCTGTTCGTATAGGTGAAGGTGAAATTCTTCTCAGA | 6906 |
| Cys29Term | TTTAAATTGGTGGCTCAAACGGAAGAAGACCCTATTCTGGGCTTTG | |
| TGC-TGA | GGTATATTCTTGGTAGTGGTCTAAGACCA | |
| | TCCGTTTGAGCCACCAA | 6907 |
| | TTGGTGGCTCAAACGGA | 6908 |
| Reduced linolenic acid | CACTACCAAGAATATACCCAAAGCCCAGAATAGGGTCTTCTTCCGT | 6909 |
| omega-3 fatty acid | TTGCGCCACCAATTGAAATCTGAGAAGAATTTCACCTTCACCTATA | |
| desaturase | CGAACAGATCGGAATTGTTGGGCATTGAG | |
| Solanum tuberosum | CTCAATGCCCAACAATTCCGATCTGTTCGTATAGGTGAAGGTGAAA | 6910 |
| Leu33Term | TTCTTCTCAGATTTCAATTGGTGGCGCAAACGGAAGAAGACCCTAT | |
| TTA-TGA | TCTGGGCTTTGGGTATATTCTTGGTAGTG | |
| | CACCAATTGAAATCTGA | 6911 |
| | TCAGATTTCAATTGGTG | 6912 |
| Reduced linolenic acid | AGAATATACCCAAAGCCCAGAATAGGGTCTTCTTCCGTTTGCGCCA | 6913 |
| omega-3 fatty acid | CCAATTTAAATCTGTGAAGAATTTCACCTTCACCTATACGAACAGAT | |
| desaturase | CGGAATTGTTGGGCATTGAGGGTAAGTG | |
| Solanum tuberosum | CACTTACCCTCAATGCCCAACAATTCCGATCTGTTCGTATAGGTGA | 6914 |
| Arg36Term | AGGTGAAATTCTTCACAGATTTAAATTGGTGGCGCAAACGGAAGAA | |
| AGA-TGA | GACCCTATTCTGGGCTTTGGGTATATTCT | |
| | TAAATCTGTGAAGAATT | 6915 |
| | AATTCTTCACAGATTTA | 6916 |
| Reduced linolenic acid | CTCTTTATTATCCTCCTCTTCTTTGTTTTTTTTGAGTTCTGAGTCACC | 6917 |
| omega-3 fatty acid | TATGGCAAGTTGAGTGATTTCAGAATGTGGGCTAAGGCCACTTCC | |
| desaturase | AAGAATCTATGCCAGGCCCAGAAGTGGA | |
| Petroselinum crispum | TCCACTTCTGGGCCTGGCATAGATTCTTGGAAGTGGCCTTAGCCC | 6918 |
| Trp4Term | ACATTCTGAAATCACTCAACTTGCCATAGGTGACTCAGAACTCATAA | |
| TGG-TGA | AAAAACAAAGAAGAGGAGGATAATAAAGAG | |
| | GCAAGTTGAGTGATTTC | 6919 |
| | GAAATCACTCAACTTGC | 6920 |
| Reduced linolenic acid | TATCCTCCTCTTCTTTGTTTTTTTTGAGTTCTGAGTCACCTATGGCA | 6921 |
| omega-3 fatty acid | AGTTGGGTGATTTGAGAATGTGGGCTAAGGCCACTTCCAAGT4ATC | |
| desaturase | TATGCCAGGCCCAGAAGTGGAGCTTCATG | |
| Petroselinum crispum | CATGAAGCTCCACTTCTGGGCCTGGCATAGATTCTTGGAAGTGGC | 6922 |
| Ser7Term | CTTAGCCCACATTCTCAAATCACCCAACTTGCCATAGGTGACTCAG | |
| TCA-TGA | AACTCAAAAAAAACAAAGAAGAGGAGGATA | |
| | GGTGATTTGAGAATGTG | 6923 |
| | CACATTCTCAAATCACC | 6924 |
| Reduced linolenic acid | TCCTCCTCTTCTTTGTTTTTTTTGAGTTCTGAGTCACCTATGGCAAG | 6925 |
| omega-3 fatty acid | TTGGGTGATTTCATAATGTGGGCTAAGGCCACTTCCAAGAATCTAT | |
| desaturase | GCCAGGCCCAGAAGTGGAGCTTCATGTT | |
| Petroselinum crispum | AACATGAAGCTCCACTTCTGGGCCTGGCATAGATTCTTGGAAGTG | 6926 |
| Glu8Term | GCCTTAGCCCACATTATGAAATCACCCAACTTGCCATAGGTGACTC | |
| GAA-TAA | AGAACTCAAAAAAAACAAAGAAGAGGAGGA | |
| | TGATTTCATAATGTGGG | 6927 |
| | CCCACATTATGAAATCA | 6928 |
| Reduced linolenic acid | CTCTTCTTTGTTTTTTTTGAGTTCTGAGTCACCTATGGCAAGTTGGG | 6929 |
| omega-3 fatty acid | TGATTTCAGAATGAGGGCTAAGGCCACTTCCAAGAATCTATGCCA | |
| desaturase | GGCCCAGAAGTGGAGCTTCATGTTTCAAC | |
| Petroselinum crispum | GTTGAAACATGAAGCTCCACTTCTGGGCCTGGCATAGATTCTTGG | 6930 |
| Cys9Term | AAGTGGCCTTAGCCCTCATTCTGAAATCACCCAACTTGCCATAGGT | |
| TGT-TGA | GACTCAGAACTCAAAAAAAACAAAGAAGAG | |
| | TCAGAATGAGGGCTAAG | 6931 |
| | CTTAGCCCTCATTCTGA | 6932 |
| Reduced linolenic acid | ATGAAGCAGCAACAGTACAAAGACACCCCAATTCTAAATGGCGTTA | 6933 |
| omega-3 fatty acid | ATGGTTTTCATGCTTAAGAAGAAGAAGAAGAAGAGGATTTCGACTT | |
| desaturase | AAGCAATCCTCCTCCATTCAATATTGGTC | |
| Vernicia fordii | GACCAATATTGAATGGAGGAGGATTGCTTAAGTCGAAATCCTCTTC | 6934 |
| Lys21Term | TTCTTCTTCTTCTTAAGCATGAAAACCATTAACGCCATTTAGAATTG | |
| AAA-TAA | GGGTGTCTTTGTACTGTTGCTGCTTCAT | |
| | TTCATGCTTAAGAAGAA | 6935 |
| | TTCTTCTTAAGCATGAA | 6936 |
| Reduced linolenic acid | AAGCAGCAACAGTACAAAGACACCCCAATTCTAAATGGCGTTAATG | 6937 |
| omega-3 fatty acid | GTTTTCATGCTAAATAAGAAGAAGAAGAAGAGGATTTCGACTTAAG | |
| desaturase | CAATCCTCCTCCATTCAATATTGGTCAGA | |
| Vernicia fordii | TCTGACCAATATTGAATGGAGGAGGATTGCTTAAGTCGAAATCCTC | 6938 |
| Glu22Term | TTCTTCTTCTTCTTATTTAGCATGAAAACCATTAACGCCATTTAGAA | |
| GAA-TAA | TTGGGGTGTCTTTGTACTGTTGCTGCTT | |
| | ATGCTAAATAAGAAGAA | 6939 |
| | TTCTTCTTATTTAGCAT | 6940 |
| Reduced linolenic acid | CAGCAACAGTACAAAGACACCCCAATTCTAAATGGCGTTAATGGTT | 6941 |
| omega-3 fatty acid | TTCATGCTAAAGAATAAGAAGAAGAAGAGGATTTCGACTTAAGCAA | |
| desaturase | TCCTCCTCCATTCAATATTGGTCAGATCC | |
| Vernicia fordii | GGATCTGACCAATATTGAATGGAGGAGGATTGCTTAAGTCGAAATC | 6942 |
| Glu23Term | CTCTTCTTCTTCTTATTCTTTAGCATGAAAACCATTAACGCCATTTA | |
| GAA-TAA | GAATTGGGGTGTCTTTGTACTGTTGCTG | |
| | CTAAAGAATAAGAAGAA | 6943 |
| | TTCTTCTTATTCTTTAG | 6944 |
| Reduced linolenic acid | CAGCAACAGTACAAAGACACCCCAATTCTAAATGGCGTTAATGGTT | 6945 |
| omega-3 fatty acid | TTCATGCTAAAGAATAAGAAGAAGAAGAGGATTTCGACTTAAGCAA | |
| desaturase | TCCTCCTCCATTCAATATTGGTCAGATCC | |
| Vernicia fordii | GGATCTGACCAATATTGAATGGAGGAGGATTGCTTAAGTCGAAATC | 6946 |
| Glu24Term | CTCTTCTTCTTCTTATTCTTTAGCATGAAAACCATTAACGCCATTTA | |
| GAA-TAA | GAATTGGGGTGTCTTTGTACTGTTGCTG | |
| | CTAAAGAATAAGAAGAA | 6947 |
| | TTCTTCTTATTCTTTAG | 6948 |
| Reduced linolenic acid | GGTCCAAGCACAGCCTCTACAACATGTTGGTAATGGTGCAGGGAA | 6949 |
| omega-3 fatty acid | AGAAGATCAAGCTTAGTTTGATCCAAGTGCTCCACCACCCTTCAAG | |
| desaturase | ATTGCAAATATCAGAGCAGCAATTCCAAAA | |
| Glycine max | TTTTGGAATTGCTGCTCTGATATTTGCAATCTTGAAGGGTGGTGGA | 6950 |
| Tyr21Term | GCACTTGGATCAAACTAAGCTTGATCTTCTTTCCCTGCACCATTAC | |
| TAT-TAG | CAACATGTTGTAGAGGCTGTGCTTGGACC | |
| | CAAGCTTAGTTTGATCC | 6951 |
| | GGATCAAACTAAGCTTG | 6952 |
| Reduced linolenic acid | GGTAATGGTGCAGGGAAAGAAGATCAAGCTTATTTTGATCCAAGT | 6953 |
| omega-3 fatty acid | GCTCCACCACCCTTCTAGATTGCAAATATCAGAGCAGCAATTCCAA | |
| desaturase | AACATTGCTGGGAGAAGAACACATTGAGAT | |
| Glycine max | ATCTCAATGTGTTCTTCTCCCAGCAATGTTTTGGAATTGCTGCTCT | 6954 |
| Lys31Term | GATATTTGCAATCTAGAAGGGTGGTGGAGCACTTGGATCAAAATAA | |
| AAG-TAG | GCTTGATCTTCTTTCCCTGCACCATTACC | |
| | CACCCTTCTAGATTGCA | 6955 |
| | TGCAATCTAGAAGGGTG | 6956 |
| Reduced linolenic acid | AAAGAAGATCAAGCTTATTTTGATCCAAGTGCTCCACCACCCTTCA | 6957 |
| omega-3 fatty acid | AGATTGCAAATATCTGAGCAGCAATTCCAAAACATTGCTGGGAGAA | |
| desaturase | GAACACATTGAGATCTCTGAGTTATGTTC | |
| Glycine max | GAACATAACTCAGAGATCTCAATGTGTTCTTCTCCCAGCAATGTTTT | 6958 |
| Arg36Term | GGAATTGCTGCTCAGATATTTGCAATCTTGAAGGGTGGTGGAGCA | |
| AGA-TGA | CTTGGATCAAAATAAGCTTGATCTTCTTT | |
| | CAAATATCTGAGCAGCA | 6959 |
| | TGCTGCTCAGATATTTG | 6960 |
| Reduced linolenic acid | TATTTTGATCCAAGTGCTCCACCACCCTTCAAGATTGCAAATATCA | 6961 |
| omega-3 fatty acid | GAGCAGCAATTCCATAACATTGCTGGGAGAAGAACACATTGAGAT | |
| desaturase | CTCTGAGTTATGTTCTGAGGGATGTGTTGG | |
| Glycine max | CCAACACATCCCTCAGAACATAACTCAGAGATCTCAATGTGTTCTT | 6962 |
| Leu41Term | CTCCCAGCAATGTTATGGAATTGCTGCTCTGATATTTGCAATCTTG | |
| AAA-TAA | AAGGGTGGTGGAGCACTTGGATCAAAATA | |
| | CAATTCCATAACATTGC | 6963 |
| | GCAATGTTATGGAATTG | 6964 |
| Reduced linolenic acid | CATCCACCCGCACCCGCACCCGCCCCGCTGACGGCGGCAATGGC | 6965 |
| omega-3 fatty acid | CCGGCTCGTGCTCTCCTAGTGCTCGGGCCTCGCGCCCGTCCGCC | |
| desaturase | GCCTGCGCGCCGGCCGGGGCGCCATTGCGGCGC | |
| Zea mays | GCGCCGCAATGGCGCCCCGGCCGGCGCGCAGGCGGCGGACGG | 6966 |
| Glu8Term | GCGCGAGGCCCGAGCACTAGGAGAGCACGAGCCGGGCCATTGC | |
| GAG-TAG | CGCCGTCAGCGGGGCGGGTGCGGGTGCGGGTGGATG | |
| | TGCTCTCCTAGTGCTCG | 6967 |
| | CGAGCACTAGGAGAGCA | 6968 |
| Reduced linolenic acid | ACCCGCACCCGCACCCGCCCCGCTGACGGCGGCAATGGCCCGG | 6969 |
| omega-3 fatty acid | CTCGTGCTCTCCGAGTGATCGGGCCTCGCGCCCGTCCGCCGCCT | |
| desaturase | GCGCGCCGGCCGGGGCGCCATTGCGGCGCGGTCA | |
| Zea mays | TGACCGCGCCGCAATGGCGCCCCGGCCGGCGCGCAGGCGGCGG | 6970 |
| Cys9Term | ACGGGCGCGAGGCCCGATCACTCGGAGAGCACGAGCCGGGCCA | |
| TGC-TGA | TTGCCGCCGTCAGCGGGGCGGGTGCGGGTGCGGGT | |
| | TCCGAGTGATCGGGCCT | 6971 |
| | AGGCCCGATCACTCGGA | 6972 |
| Reduced linolenic acid | CCGCACCCGCACCCGCCCCGCTGACGGCGGCAATGGCCCGGCT | 6973 |
| omega-3 fatty acid | CGTGCTCTCCGAGTGCTAGGGCCTCGCGCCCGTCCGCCGCCTGC | |
| desaturase | GCGCCGGCCGGGGCGCCATTGCGGCGCGGTCACC | |
| Zea mays | GGTGACCGCGCCGCAATGGCGCCCCGGCCGGCGCGCAGGCGGC | 6974 |
| Ser10Term | GGACGGGCGCGAGGCCCTAGCACTCGGAGAGCACGAGCCGGGC | |
| TCG-TAG | CATTGCCGCCGTCAGCGGGGCGGGTGCGGGTGCGG | |
| | CGAGTGCTAGGGCCTCG | 6975 |
| | CGAGGCCCTAGCACTCG | 6976 |
| Reduced linolenic acid | GCTCGGGCCTCGCGCCCGTCCGCCGCCTGCGCGCCGGCCGGGG | 6977 |
| omega-3 fatty acid | CGCCATTGCGGCGCGGTGACCCCCCGCGCTCTCCGCGGCGCCG | |
| desaturase | CGCCGTCGTCCCGCGTCCGCGTCCATCCACCGCGA | |
| Zea mays | TCGCGGTGGATGGACGCGGACGCGGGACGACGGCGCGGCGCCG | 6978 |
| Ser29Term | CGGAGAGCGCGGGGGGTCACCGCGCCGCAATGGCGCCCCGGCC | |
| TCA-TGA | GGCGCGCAGGCGGCGGACGGGCGCGAGGCCCGAGC | |
| | GGCGCGGTGACCCCCCG | 6979 |
| | CGGGGGGTCACCGCGCC | 6980 |
| Reduced linolenic acid | CCCCCTCCCCCACGCACACGCACAGATCCATCCGCGGCCATGGC | 6981 |
| omega-3 fatty acid | CCCCGCAATGAGGCCGTAGCAGGAGGCGAGCTGCAAGGCCACCG | |
| desaturase | AGGACCACCGCTCCGAGTTCGACGCCGCCAAGC | |
| Triticum aestivum | GCTTGGCGGCGTCGAACTCGGAGCGGTGGTCCTCGGTGGCCTTG | 6982 |
| Glu8Term | CAGCTCGCCTCCTGCTACGGCCTCATTGCGGGGGCCATGGCCGC | |
| GAG-TAG | GGATGGATCTGTGCGTGTGCGTGGGGGAGGGGG | |
| | TGAGGCCGTAGCAGGAG | 6983 |
| | CTCCTGCTACGGCCTCA | 6984 |
| Reduced linolenic acid | CCTCCCCCACGCACACGCACAGATCCATCCGCGGCCATGGCCCC | 6985 |
| omega-3 fatty acid | CGCAATGAGGCCGGAGTAGGAGGCGAGCTGCTAAGGCCACCGAG | |
| desaturase | GACCACCGCTCCGAGTTCGACGCCGCCAAGCCGC | |
| Triticum aestivum | GCGGCTTGGCGGCGTCGAACTCGGAGCGGTGGTCCTCGGTGGCC | 6986 |
| Gln9Term | TTGCAGCTCGCCTCCTACTCCGGCCTCATTGCGGGGGCCATGGC | |
| CAG-TAG | CGCGGATGGATCTGTGCGTGTGCGTGGGGGAGG | |
| | GGCCGGAGTAGGAGGCG | 6987 |
| | CGCCTCCTACTCCGGCC | 6988 |
| Reduced linolenic acid | CCCCCACGCACACGCACAGATCCATCCGCGGCCATGGCCCCCGC | 6989 |
| omega-3 fatty acid | AATGAGGCCGGAGCAGTAGGCGAGCTGCAAGGCCACCGAGGACC | |
| desaturase | ACCGCTCCGAGTTCGACGCCGCCAAGCCGCCGC | |
| Triticum aestivum | GCGGCGGCTTGGCGGCGTCGAACTCGGAGCGGTGGTCCTCGGT | 6990 |
| Glu10Term | GGCCTTGCAGCTCGCCTACTGCTCCGGCCTCATTGCGGGGGCCA | |
| GAG-TAG | TGGCCGCGGATGGATCTGTGCGTGTGCGTGGGGG | |
| | CGGAGCAGTAGGCGAGC | 6991 |
| | GCTCGCCTACTGCTCCG | 6992 |
| Reduced linolenic acid | ACGCACAGATCCATCCGCGGCCATGGCCCCCGCAATGAGGCCGG | 6993 |
| omega-3 fatty acid | AGCAGGAGGCGAGCTGAAAGGCCACCGAGGACCACCGCTCCGA | |
| desaturase | GTTCGACGCCGCCAAGCCGCCGCCCTTCCGCATC | |
| Triticum aestivum | GATGCGGAAGGGCGGCGGCTTGGCGGCGTCGAACTCGGAGCGG | 6994 |
| Cys13Term | TGGTCCTCGGTGGCCTTTCAGCTCGCCTCCTGCTCCGGCCTCATT | |
| TGC-TGA | GCGGGGGCCATGGCCGCGGATGGATCTGTGCGT | |
| | GCGAGCTGAAAGGCCAC | 6995 |
| | GTGGCCTTTCAGCTCGC | 6996 |
| Reduced linolenic acid | CTTCACAAATCACAAATCGGAATCAGATCCACCACGACACCCCGG | 6997 |
| omega-3 fatty acid | CGGCAATGGCGGCGTAGGCGACCCAGGAGGCCGACTGCAAGGC | |
| desaturase | TTCCGAGGACGCCCGTCTCTTCTTCGACGCCGC | |
| Oryza sativa | GCGGCGTCGAAGAAGAGACGGGCGTCCTCGGAAGCCTTGCAGTC | 6998 |
| Ser4Term | GGCCTCCTGGGTCGCCTACGCCGCCATTGCCGCCGGGGTGTCGT | |
| TCG-TAG | GGTGGATCTGATTCCGATTTGTGATTTGTGAAG | |
| | GGCGGCGTAGGCGACCC | 6999 |
| | GGGTCGCCTACGCCGCC | 7000 |
| Reduced linolenic acid | ATCACAAATCGGAATCAGATCCACCACGACACCCCGGCGGCAATG | 7001 |
| omega-3 fatty acid | GCGGCGTCGGCGACCTAGGAGGCCGACTGCAAGGCTTCCGAGGA | |
| desaturase | CGCCCGTCTCTTCTTCGACGCCGCCAAGCCCC | |
| Oryza sativa | GGGGCTTGGCGGCGTCGAAGAAGAGACGGGCGTCCTCGGAAGC | 7002 |
| Gln7Term | CTTGCAGTCGGCCTCCTAGGTCGCCGACGCCGCCATTGCCGCCG | |
| CAG-TAG | GGGTGTCGTGGTGGATCTGATTCCGATTTGTGAT | |
| | CGGCGACCTAGGAGGCC | 7003 |
| | GGCCTCCTAGGTCGCCG | 7004 |
| Reduced linolenic acid | ACAAATCGGAATCAGATCCACCACGACACCCCGGCGGCAATGGC | 7005 |
| omega-3 fatty acid | GGCGTCGGCGACCCAGTAGGCCGACTGCAAGGCTTCCGAGGACG | |
| desaturase | CCCGTCTCTTCTTCGACGCCGCCAAGCCCCCGC | |
| Oryza sativa | GCGGGGGCTTGGCGGCGTCGAAGAAGAGACGGGCGTCCTCGGA | 7006 |
| Glu8Term | AGCCTTGCAGTCGGCCTACTGGGTCGCCGACGCCGCCATTGCCG | |
| GAG-TAG | CCGGGGTGTCGTGGTGGATCTGATTCCGATTTGT | |
| | CGACCCAGTAGGCCGAC | 7007 |
| | GTCGGCCTACTGGGTCG | 7008 |
| Reduced linolenic acid | TCAGATCCACCACGACACCCCGGCGGCAATGGCGGCGTCGGCGA | 7009 |
| omega-3 fatty acid | CCCAGGAGGCCGACTGAAAGGCTTCCGAGGACGCCCGTCTCTTC | |
| desaturase | TTCGACGCCGCCAAGCCCCCGCCCTTCCGCATC | |
| Oryza sativa | GATGCGGAAGGGCGGGGGCTTGGCGGCGTCGAAGAAGAGACGG | 7010 |
| Cys10Term | GCGTCCTCGGAAGCCTTTCAGTCGGCCTCCTGGGTCGCCGACGC | |
| TGC-TGA | CGCCATTGCCGCCGGGGTGTCGTGGTGGATCTGA | |
| | GCCGACTGAAAGGCTTC | 7011 |
| | GAAGCCTTTCAGTCGGC | 7012 |

Although a number of embodiments and features are described herein, it will be understood by those skilled in the art that modification and variations of the described embodiments and features may be made without departing from either the spirit of the invention or the scope of the appended claims. All publications and patents cited herein are incorporated by reference in their entireties.

What is claimed is:

1. A method for identifying a cell having a desired oligonucleotide-directed sequence alteration at a first nucleic acid target site within the cell, the method comprising:
identifying said desired sequence alteration in cells that have been selected for the presence of a selectable phenotype conferred by a concurrent oligonucleotide-directed sequence alteration at a second nucleic acid target site within said cells.

2. A method for effecting a desired sequence alteration at a first nucleic acid target site within a cell, the method comprising:
concurrently targeting first and second nucleic acid sites within said cell for sequence alteration with respective first and second sequence-altering oligonucleotides, wherein said second alteration confers a selectable phenotype upon said cell;
selecting cells having said selectable phenotype; and then
identifying among the selected cells a cell having the desired sequence alteration at said first nucleic acid target site.

3. The method of claim 1 or 2, wherein said selectable phenotype is selected from the group consisting of: antibiotic resistance, prototrophy, expression of a fluorescent protein, presence of an epitope, and resistance to an apoptotic signal.

4. The method of claim 1 or 2, wherein the nucleic acid molecule comprising the first nucleic acid target does not comprise the second nucleic acid target.

5. The method of claim 1 or 2, wherein the nucleic acid molecule comprising the first nucleic acid target comprises the second nucleic acid target.

6. The method of claim 1 or 2, wherein the first nucleic acid target site is in a DNA molecule selected from the group consisting of: a chromosome, a plasmid, a YAC, a BAC, a PLAC, a MAC, and a PAC.

7. The method of claim 1 or 2, wherein the cell is selected from the group consisting of: a prokaryotic cell, a fungal cell, a plant cell, an animal cell, and a mammalian cell.

8. A composition for effecting a desired sequence alteration at a first nucleic acid target site within a cell, comprising:
first and second sequence-altering oligonucleotides, wherein the oligonucleotides are capable of effecting sequence alteration at the first nucleic acid target site and at a second nucleic acid target site, respectively; and
wherein alteration of the second nucleic acid target site confers a selectable phenotype.

9. The composition of claim 8, wherein said selectable phenotype is selected from the group consisting of: antibiotic resistance, prototrophy, expression of a fluorescent protein, presence of an epitope, and resistance to an apoptotic signal.

10. The composition of claim 8 further comprising a cellular repair protein.

11. The composition of claim 8, further comprising a cell selected from the group consisting of: a prokaryotic cell, a fungal cell, a plant cell, an animal cell, and a mammalian cell.

12. A kit for effecting a desired sequence alteration at a first nucleic acid target site within a cell, comprising:
first and second sequence-altering oligonucleotides, wherein the oligonucleotides are capable of effecting sequence alteration at the first nucleic acid target site and at a second nucleic acid target site, respectively; and
wherein alteration of the second nucleic acid target site confers a selectable phenotype.

13. The kit of claim 12 further comprising a cellular repair protein.

14. The kit of claim 12, wherein the cellular repair protein is selected from the group consisting of: RAD10, RAD51, RAD52, RAD54, RAD55, MRE11, PMS1 and XRS2.

15. The kit of claim 12 further comprising an HDAC inhibitor; hydroxyurea or lambda phage beta protein.

16. The kit of claim 12 further comprising a cell selected from the group consisting of: a prokaryotic cell, a fungal cell, a plant cell, an animal cell, and a mammalian cell.

17. The kit of claim 16, wherein the cell has increased levels or activity of at least one protein selected from the group consisting of: RAD10, RAD51, RAD52, RAD54, RAD55, MRE11, PMS1 and XRS2.

18. The kit of claim 16, wherein the cell has decreased levels or activity of at least one protein selected from the group consisting of: RAD10, RAD51, RAD52, RAD54, RAD55, MRE11, PMS1 and XRS2.

19. The kit of claim 16, wherein the cell comprises a target nucleic acid sequence, wherein alteration of said target nucleic acid sequence by said second oligonucleotide confers a selectable phenotype.

20. The kit of claim 12, wherein the kit further comprises instructions for performing the method of claim 1 or claim 2.
